(19) United States
(12) Patent Application Publication
(10) Pub. No.: US 2007/0198063 A1
(43) Pub. Date: Aug. 23, 2007

(54) Electrical devices and anti-scarring drug combinations

(76) Inventors: William L. Hunter, Vancouver (CA); Philip M. Toleikis, Vancouver (CA); David M. Gravett, Vancouver (CA); Daniel S. Grau, Arlington, MA (US); Alexis Borisy, Arlington, MA (US); Curtis T. Keith, Boston, MA (US); Benjamin A. Auspitz, Cambridge, MA (US); M. James Nichols, Boston, MA (US); Edward Roydon Jost-Price, West Roxbury, MA (US); George N. Serbedzija, Sudbury, MA (US)

Correspondence Address: CLARK & ELBING LLP, 101 FEDERAL STREET, BOSTON, MA, 02110, US

(21) Appl. No.: 11/542,163

(22) Filed: Oct. 3, 2006

Related U.S. Application Data
(60) Provisional application No. 60/723,637, filed on Oct. 3, 2005.

(51) Int. Cl. A61N 1/18

(52) U.S. Cl. 607/3

(57) ABSTRACT

Electrical devices (e.g., cardiac rhythm management and neurostimulation devices) for contact with tissue are used in combination with an anti-scarring drug combination or a composition that comprises an anti-scarring drug combination to inhibit scarring that may otherwise occur when the devices are implanted within an animal.

### CROSS-REFERENCE TO RELATED APPLICATIONS

This application claims benefit of U.S. Provisional Application No. 60/723,637, filed Oct. 3, 2005; which is hereby incorporated by reference.

### FIELD OF THE INVENTION

The present invention relates generally to electrical devices and pharmaceutical compositions that include anti-scarring drug combinations and to methods for making and using such devices and compositions.

### DESCRIPTION OF THE RELATED ART

Medical devices having electrical components, such as electrical pacing or stimulating devices, can be implanted in the body to provide electrical conduction to the central and peripheral nervous system (including the autonomic system), cardiac muscle tissue (including myocardial conduction pathways), smooth muscle tissue and skeletal muscle tissue. These electrical impulses are used to treat many bodily dysfunctions and disorders by blocking, masking, stimulating, or replacing electrical signals within the body. Examples include pacemaker leads used to maintain the normal rhythmic beating of the heart; defibrillator leads used to “re-start” the heart when it stops beating; peripheral nerve stimulating devices to treat chronic pain; deep brain electrical stimulation to treat conditions such as tremor, Parkinson's disease, movement disorders, epilepsy, depression and psychiatric disorders; and vagal nerve stimulation to treat epilepsy, depression, anxiety, obesity, migraine and Alzheimer's Disease.

The clinical function of an electrical device such as a cardiac pacemaker lead, neurostimulation lead, or other electrical lead depends upon the device being able to effectively maintain intimate anatomical contact with the target tissue (typically electrically excitable cells such as muscle or nerve) such that electrical conduction from the device to the tissue can occur. Unfortunately, in many instances when these devices are implanted in the body, they are subject to a “foreign body” response from the surrounding host tissues. The body recognizes the implanted device as foreign, which triggers an inflammatory response followed by encapsulation of the implant with fibrous connective tissue (or glial tissue—called “gliosis”—when it occurs within the central nervous system). Scarring (i.e., fibrosis or gliosis) can also result from trauma to the anatomical structures and tissue surrounding the implant during the implantation of the device. Lastly, fibrous encapsulation of the device can occur even after a successful implantation if the device is manipulated (some patients continuously “fiddle” with a subcutaneous implant) or irritated by the daily activities of the patient. When scarring occurs around the implanted device, the electrical characteristics of the electrode-tissue interface degrade, and the device may fail to function properly. For example, it may require additional electrical current from the lead to overcome the extra resistance imposed by the intervening scar (or glial) tissue. This can shorten the battery life of an implant (making more frequent removal and re-implantation necessary), prevent electrical conduction altogether (rendering the implant clinically ineffective) and/or cause damage to the target tissue. Additionally, the surrounding tissue may be inadvertently damaged from the inflammatory foreign body response, which can result in loss of function or tissue necrosis.

### BRIEF SUMMARY OF THE INVENTION

Briefly stated, the present invention discloses drug combinations, or individual component(s) thereof, which inhibit one or more aspects of the production of excessive fibrous or glial (scar) tissue. In one aspect, the present invention provides compositions for delivery of selected anti-scarring drug combinations, or individual component(s) thereof, via medical implants or implantable electrical medical devices, as well as methods for making and using these implants and devices. Compositions and methods are described for coating electrical medical devices and implants with anti-scarring drug combinations, or individual component(s) thereof, such that anti-scarring drug combinations, or individual component(s) thereof, are delivered in therapeutic levels over a period sufficient to prevent the device electrode from being encapsulated in fibrous or glial tissue and to allow normal electrical conduction to occur. Alternatively, locally administered compositions (e.g., topicals, injectables, liquids, gels, sprays, microspheres, pastes, wafers) containing an inhibitor of fibrosis (or gliosis) are described that can be applied to the tissue adjacent to the electrical medical device or implant, such that the fibrosis-inhibiting or gliosis-inhibiting drug combination, or individual component(s) thereof, is delivered in therapeutic levels over a period sufficient to prevent the device electrode from being encapsulated in fibrous or glial tissue. And finally, numerous specific cardiac and neurological implants and devices are described that produce superior clinical results as a result of being coated with agents that reduce excessive scarring and fibrous (or glial) tissue accumulation, as well as other related advantages.

Within one aspect of the invention, drug-coated or drug-impregnated implants and medical coated or impregnated with anti-fibrosis (or anti-gliosis) drug combinations, or individual component(s) thereof, are provided which reduce fibrosis or gliosis in the tissue surrounding the electrical device or implant, or inhibit scar development on the device/implant surface (particularly the electrical lead), thus enhancing the efficacy of the procedure. For example, additional electrical current from the lead may be required to overcome the extra resistance imposed by the intervening fibrotic (or glial) tissue. This can shorten the battery life of an implant (making more frequent removal and re-implantation necessary), prevent electrical conduction altogether (rendering the implant clinically ineffective) and/or cause damage to the target tissue. Within various embodiments, fibrosis or gliosis is inhibited by local or systemic release of specific drug combinations, or individual component(s) thereof, that become localized to the adjacent tissue.

The repair of tissues following a mechanical or surgical intervention, such as the implantation of an electrical device, involves two distinct processes: (1) regeneration (the replacement of injured cells by cells of the same type) and (2) fibrosis (the replacement of injured cells by connective tissue). There are several general components to the process of fibrosis (or scarring) including: infiltration of inflammatory cells and the inflammatory response, migration and proliferation of connective tissue cells (such as fibroblasts or smooth muscle cells), deposition of extracellular matrix (ECM), formation of new blood vessels (angiogenesis), and remodeling (maturation and organization of the fibrous tissue). As utilized herein, “inhibits (reduces) fibrosis” should be understood to refer to agents or compositions which decrease or limit the formation of fibrous tissue (i.e., by reducing or inhibiting one or more of the processes of angiogenesis, connective tissue cell migration or proliferation, ECM production, and/or remodeling). In addition, numerous therapeutic agents described in this invention may have the additional benefit of also reducing tissue regeneration where appropriate.

It should be noted that in implantation procedures that cause injuries to the central nervous system (CNS), fibrosis is replaced by a process called gliosis (the replacement of injured or dead cells with glial tissue). Glial cells form the supporting tissue of the CNS and are comprised of macroglia (astrocytes, oligodendrocytes, ependyma cells) and microglia cells. Of these cell types, astrocytes are the principle cells responsible for repair and scar formation in the brain and spinal cord. Gliosis is the most important indicator of CNS damage and consists of astrocyte hypertrophy (increase in size) and hyperplasia (increase in cell number as a result of cell division) in response to injury or trauma, such as that caused by the implantation of a medical device. Astrocytes are responsible for phagocytosing dead or damaged tissue and repairing the injury with glial tissue and thus, serve a similar role to that performed by fibroblasts in scarring outside the brain. In medical devices implanted into the CNS, it is the hypertrophy and proliferation of astrocytes (gliosis) that leads to the formation of a “scar-like” capsule around the implant which can interfere with electrical conduction from the device to the neuronal tissue.

Within certain embodiments of the invention, an implant or device is adapted to release a drug combination, or individual component(s) thereof, that inhibits fibrosis or gliosis through one or more of the mechanisms cited herein. Within certain other embodiments of the invention, an implant or device contains a drug combination, or individual component(s) thereof, that, while remaining associated with the implant or device, inhibits fibrosis or gliosis between the implant or device and the tissue where the implant or device is placed by direct contact between the drug combination and the tissue surrounding the implant or device.

Within related aspects of the present invention, cardiac and neurostimulation devices are provided comprising an implant or device, wherein the implant or device releases a drug combination, or individual component(s) thereof, which inhibits fibrosis (or gliosis) in vivo. Within yet other aspects of the present invention, methods are provided for manufacturing a medical device or implant, comprising the step of coating (e.g., spraying, dipping, wrapping, or administering drug through) a medical device or implant with anti-fibrosis (or anti-gliosis) drug combination(s), or individual component(s) thereof. Additionally, the implant or medical device can be constructed so that the device itself is comprised of materials that comprise anti-fibrosis (or anti-gliosis) drug combinations, or individual component(s) thereof, in or around the implant. A wide variety of electrical medical devices and implants may be utilized within the context of the present invention, depending on the site and nature of treatment desired.

Within various embodiments of the invention, the implant or device is further coated with a composition or compound, which delays the onset of activity of the fibrosis-inhibiting (or gliosis-inhibiting) drug combinations, or individual component(s) thereof, for a period of time after implantation. Representative examples of such agents include heparin, PLGA/MePEG, PLA, and polyethylene glycol. Within further embodiments, the fibrosis-inhibiting (or gliosis-inhibiting) implant or device is activated before, during, or after deployment (e.g., an inactive agent on the device is first activated to one that reduces or inhibits an in vivo fibrotic or gliotic reaction).

Within various embodiments of the invention, the tissue surrounding the implant or device is treated with a composition or compound that contains a drug combination, or individual component(s) thereof, that inhibits fibrosis or gliosis. Locally administered compositions (e.g., topicals, injectables, liquids, gels, sprays, microspheres, pastes, wafers) or compounds containing an inhibitor of fibrosis (or gliosis) are described that can be applied to the surface of, or infiltrated into, the tissue adjacent to the electrical medical device or implant, such that the pharmaceutical agent is delivered in therapeutic levels over a period sufficient to prevent the device electrode from being encapsulated in fibrous or glial tissue. This can be done in lieu of coating the device or implant with a fibrosis/gliosis-inhibitor, or done in addition to coating the device or implant with a fibrosis/gliosis-inhibitor. The local administration of the fibrosis/gliosis-inhibiting drug combination, or individual component(s) thereof, can occur prior to, during, or after implantation of the electrical device itself.

Within various embodiments of the invention, an electrical device or implant is coated on one aspect, portion or surface with a composition which inhibits fibrosis or gliosis, as well as being coated with a composition or compound which promotes scarring on another aspect, portion or surface of the device (i.e., to affix the body of the device into a particular anatomical space). Representative examples of agents that promote fibrosis and scarring include silk, silica, crystalline silicates, bleomycin, quartz dust, neomycin, talc, metallic beryllium and oxides thereof, retinoic acid compounds, copper, leptin, growth factors, a component of extracellular matrix; fibronectin, collagen, fibrin, or fibrinogen, polylysine, poly(ethylene-co-vinylacetate), chitosan, N-carboxybutylchitosan, and RGD proteins; vinyl chloride or a polymer of vinyl chloride; an adhesive selected from the group consisting of cyanoacrylates and crosslinked poly(ethylene glycol)-methylated collagen; an inflammatory cytokine (e.g., TGFβ, PDGF, VEGF, bFGF, TNFα, NGF, GM-CSF, IGF-1, IL-1, IL-1-β, IL-8, IL-6, and growth hormone); connective tissue growth factor (CTGF) as well as analogues and derivatives thereof.

Also provided by the present invention are methods for treating patients undergoing surgical, endoscopic or minimally invasive therapies where an electrical device or implant is placed as part of the procedure. As utilized herein, it should be understood that “inhibits fibrosis or gliosis” refers to a statistically significant decrease in the amount of scar tissue in or around the device or an improvement in the interface between the electrical device or implant and the tissue, which may or may not lead to a permanent prohibition of any complications or failures of the device/implant.

The fibrosis-inhibiting (or gliosis-inhibiting) drug combinations, or individual component(s) thereof, and compositions that comprising fibrosis-inhibiting (or gliosis-inhibiting) drug combinations, or individual component(s) thereof, are utilized to create novel drug-coated implants and medical devices that reduce the foreign body response to implantation and limit the growth of reactive tissue on the surface of, or around in the tissue surrounding the device, such that performance is enhanced. Electrical medical devices and implants coated with selected pharmaceutical agents designed to prevent scar tissue overgrowth and improve electrical conduction can offer significant clinical advantages over uncoated devices.

For example, in one aspect the present invention is directed to electrical stimulatory devices that comprise a medical implant and at least one of (i) an anti-scarring drug combination, or individual component(s) thereof, and (ii) a composition that comprises an anti-scarring drug combination, or individual component(s) thereof. The anti-scarring drug combination, or individual component(s) thereof, is present so as to inhibit scarring that may otherwise occur when the implant is placed within an animal. In another aspect the present invention is directed to methods wherein both an implant and at least one of (i) an anti-scarring drug combination, or individual component(s) thereof, and (ii) a composition that comprises an anti-scarring drug combination, or individual component(s) thereof, are placed into an animal, and the anti-scarring drug combination, or individual component(s) thereof, inhibits scarring that may otherwise occur. These and other aspects of the invention are summarized below.

Thus, in various independent aspects, the present invention provides a device, comprising a cardiac or neurostimulator implant and an anti-scarring drug combination, or individual component(s) thereof, or a composition comprising an anti-scarring drug combination, or individual component(s) thereof, wherein the drug combination, or individual component(s) thereof, inhibits scarring. These and other devices are described in more detail herein.

In additional aspects, for each of the aforementioned devices combined with each of the drug combinations, or individual component(s) thereof, described herein, it is, for each combination, independently disclosed that the drug combination may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and drug combination described above, are set forth in greater detail herein.

In addition to devices, the present invention also provides methods. For example, in additional aspects of the present invention, for each of the aforementioned devices, and for each of the aforementioned combinations of the devices with the anti-fibrotic (or anti-gliotic) drug combination, or individual component(s) thereof, the present invention provides methods whereby a specified device is implanted into an animal, and a specified drug combination associated with the device inhibits scarring (or gliosis) that may otherwise occur. Each of the devices identified herein may be a “specified device”, and each of the anti-scarring drug combinations identified herein may be an “anti-scarring drug combination”, where the present invention provides, in independent embodiments, for each possible combination of the device and the drug combination.

The drug combination, or individual component(s) thereof, may be associated with the device prior to the device being placed within the animal. For example, the drug combination, or individual component(s) thereof, or a composition comprising the drug combination, or individual component(s) thereof, may be coated onto an implant, and the resulting device then placed within the animal. In addition, or alternatively, the drug combination, or individual component(s) thereof, may be independently placed within the animal in the vicinity of where the device is to be, or is being, placed within the animal. For example, the drug combination, or individual component(s) thereof, may be sprayed or otherwise placed onto, adjacent to, and/or within the tissue that will be contacting the medical implant or may otherwise undergo scarring. To this end, the present invention provides placing a cardiac or neurostimulation implant and an anti-scarring (or anti-gliosis) drug combination, or individual component(s) thereof, or a composition comprising an anti-scarring (or anti-gliosis) drug combination, or individual component(s) thereof, into an animal host, wherein the drug combination inhibits fibrosis or gliosis.

In additional aspects, for each of the aforementioned methods used in combination with each of the agents drug combinations, or individual component(s) thereof, described herein, it is, for each combination, independently disclosed that the agent drug combinations, or individual component(s) thereof, may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and agent described above, are set forth in greater detail herein.

In each of the aforementioned devices, compositions, methods of making the aforementioned devices or compositions, and methods of using the aforementioned devices or compositions, the present invention provides that the anti-fibrotic (or anti-gliotic) drug combination may be one or more of the following: 1) an anti-fibrotic (or anti-gliotic) drug combination that inhibits cell regeneration, 2) an anti-fibrotic (or anti-gliotic) drug combination that inhibits angiogenesis, 3) an anti-fibrotic (or anti-gliotic) drug combination that inhibits cell migration (e.g., fibroblasts, glial cells, smooth muscle cells, etc.), 4) an anti-fibrotic (or anti-gliotic) drug combination that inhibits cell proliferation (e.g., fibroblasts, glial cells, smooth muscle cells, etc.), 5) an anti-fibrotic (or anti-gliotic) drug combination that inhibits deposition of extracellular matrix, 6) an anti-fibrotic (or anti-gliotic) drug combination that inhibits tissue remodeling, 7) an anti-fibrotic (or anti-gliotic) drug combination that inhibits cytokine (e.g., TNF-alpha, IL-1, etc.) and/or chemokine (e.g., MCP-1) production or effects.

In certain independent aspects, the present invention provides a medical device, comprising an electrical device and an anti-scarring drug combination or a composition comprising an anti-scarring drug combination, wherein the drug combination inhibits scarring between the medical device and the host into which the medical device is implanted; a medical device, comprising a neurostimulator for treating chronic pain (i.e., an electrical device) and an anti-scarring drug combination or a composition comprising an anti-scarring drug combination, wherein the drug combination inhibits scarring between the medical device and the host into which the medical device is implanted; a medical device, comprising a neurostimulator for treating Parkinson's Disease (i.e., an electrical device) and an anti-scarring drug combination or a composition comprising an anti-scarring drug combination, wherein the drug combination inhibits scarring between the medical device and the host into which the medical device is implanted; a medical device, comprising a vagal nerve stimulator for treating epilepsy (i.e., an electrical device) and an anti-scarring drug combination or a composition comprising an anti-scarring drug combination, wherein the drug combination inhibits scarring between the medical device and the host into which the medical device is implanted; a medical device, comprising a vagal nerve stimulator (i.e., an electrical device) and an anti-scarring drug combination or a composition comprising an anti-scarring drug combination, wherein the drug combination inhibits scarring between the medical device and the host into which the medical device is implanted; a medical device, comprising a sacral nerve stimulator for treating a bladder control problem (i.e., an electrical device) and an anti-scarring drug combination or a composition comprising an anti-scarring drug combination, wherein the drug combination inhibits scarring between the medical device and the host into which the medical device is implanted; a medical device, comprising a gastric nerve stimulator for treating a gastrointestinal disorder (i.e., an electrical device) and an anti-scarring drug combination or a composition comprising an anti-scarring drug combination, wherein the drug combination inhibits scarring between the medical device and the host into which the medical device is implanted; a medical device, comprising a cochlear implant for treating deafness (i.e., an electrical device) and an anti-scarring drug combination or a composition comprising an anti-scarring drug combination, wherein the drug combination inhibits scarring between the medical device and the host into which the medical device is implanted; a medical device, comprising a bone growth stimulator (i.e., an electrical device) and an anti-scarring drug combination or a composition comprising an anti-scarring drug combination, wherein the drug combination inhibits scarring between the medical device and the host into which the medical device is implanted; a medical device, comprising a cardiac pacemaker (i.e., an electrical device) and an anti-scarring drug combination or a composition comprising an anti-scarring drug combination, wherein the drug combination inhibits scarring between the medical device and the host into which the medical device is implanted; a medical device, comprising an implantable cardioverter defibrillator (ICD) system (i.e., an electrical device) and an anti-scarring drug combination or a composition comprising an anti-scarring drug combination, wherein the drug combination inhibits scarring between the medical device and the host into which the medical device is implanted; a medical device, comprising a vagus nerve stimulator for treating arrhythmia (i.e., an electrical device) and an anti-scarring drug combination or a composition comprising an anti-scarring drug combination, wherein the drug combination inhibits scarring between the medical device and the host into which the medical device is implanted; a medical device, comprising an electrical lead (i.e., an electrical device) and an anti-scarring drug combination or a composition comprising an anti-scarring drug combination, wherein the drug combination inhibits scarring between the medical device and the host into which the medical device is implanted; a medical device, comprising a neurostimulator (i.e., an electrical device) and an anti-scarring drug combination or a composition comprising an anti-scarring drug combination, wherein the drug combination inhibits scarring between the medical device and the host into which the medical device is implanted; and a medical device, comprising a cardiac rhythm management device (i.e., an electrical device) and an anti-scarring drug combination or a composition comprising an anti-scarring drug combination, wherein the drug combination inhibits scarring between the medical device and the host into which the medical device is implanted.

In certain independent aspects, the present invention provides a method for inhibiting scarring comprising placing an electrical device and an anti-scarring drug combination or a composition comprising an anti-scarring drug combination into an animal host, wherein the drug combination inhibits scarring; a method for inhibiting scarring comprising placing a neurostimulator for treating chronic pain (i.e., an electrical device) and an anti-scarring drug combination or a composition comprising an anti-scarring drug combination into an animal host, wherein the drug combination inhibits scarring; a method for inhibiting scarring comprising placing a neurostimulator for treating Parkinson's Disease (i.e., an electrical device) and an anti-scarring drug combination or a composition comprising an anti-scarring drug combination into an animal host, wherein the drug combination inhibits scarring; a method for inhibiting scarring comprising placing a vagal nerve stimulator for treating epilepsy (i.e., an electrical device) and an anti-scarring drug combination or a composition comprising an anti-scarring drug combination into an animal host, wherein the drug combination inhibits scarring; a method for inhibiting scarring comprising placing a vagal nerve stimulator (i.e., an electrical device) and an anti-scarring drug combination or a composition comprising an anti-scarring drug combination into an animal host, wherein the drug combination inhibits scarring; a method for inhibiting scarring comprising placing a sacral nerve stimulator for treating a bladder control problem (i.e., an electrical device) and an anti-scarring drug combination or a composition comprising an anti-scarring drug combination into an animal host, wherein the drug combination inhibits scarring; a method for inhibiting scarring comprising placing a gastric nerve stimulator for treating a gastrointestinal disorder (i.e., an electrical device) and an anti-scarring drug combination or a composition comprising an anti-scarring drug combination into an animal host, wherein the drug combination inhibits scarring; a method for inhibiting scarring comprising placing a cochlear implant for treating deafness (i.e., an electrical device) and an anti-scarring drug combination or a composition comprising an anti-scarring agent into an animal host, wherein the drug combination inhibits scarring; a method for inhibiting scarring comprising placing a bone growth stimulator (i.e., an electrical device) and an anti-scarring drug combination or a composition comprising an anti-scarring drug combination into an animal host, wherein the drug combination inhibits scarring; a method for inhibiting scarring comprising placing a cardiac pacemaker (i.e., an electrical device) and an anti-scarring drug combination or a composition comprising an anti-scarring drug combination into an animal host, wherein the drug combination inhibits scarring; a method for inhibiting scarring comprising placing an implantable cardioverter defibrillator (ICD) system (i.e., an electrical device) and an anti-scarring drug combination or a composition comprising an anti-scarring drug combination into an animal host, wherein the drug combination inhibits scarring; a method for inhibiting scarring comprising placing a vagus nerve stimulator for treating arrhythmia (i.e., an electrical device) and an anti-scarring drug combination or a composition comprising an anti-scarring drug combination into an animal host, wherein the drug combination inhibits scarring; a method for inhibiting scarring comprising placing an electrical lead (i.e., an electrical device) and an anti-scarring drug combination or a composition comprising an anti-scarring drug combination into an animal host, wherein the drug combination inhibits scarring; a method for inhibiting scarring comprising placing a neurostimulator (i.e., an electrical device) and an anti-scarring drug combination or a composition comprising an anti-scarring drug combination into an animal host, wherein the drug combination inhibits scarring; and a method for inhibiting scarring comprising placing a cardiac rhythm management device (i.e., an electrical device) and an anti-scarring drug combination or a composition comprising an anti-scarring drug combination into an animal host, wherein the drug combination inhibits scarring.

In certain independent aspects, the present invention provides a method for making a medical device comprising: combining an electrical device and an anti-scarring drug combination or a composition comprising an anti-scarring drug combination, wherein the drug combination inhibits scarring between the device and a host into which the device is implanted; a method for making a medical device comprising: combining a neurostimulator for treating chronic pain (i.e., an electrical device) and an anti-scarring drug combination or a composition comprising an anti-scarring drug combination, wherein the drug combination inhibits scarring between the device and a host into which the device is implanted; a method for making a medical device comprising: combining a neurostimulator for treating Parkinson's Disease (i.e., an electrical device) and an anti-scarring drug combination or a composition comprising an anti-scarring drug combination, wherein the drug combination inhibits scarring between the device and a host into which the device is implanted; a method for making a medical device comprising: combining a vagal nerve stimulator for treating epilepsy (i.e., an electrical device) and an anti-scarring drug combination or a composition comprising an anti-scarring drug combination, wherein the drug combination inhibits scarring between the device and a host into which the device is implanted; a method for making a medical device comprising: combining a vagal nerve stimulator (i.e., an electrical device) and an anti-scarring drug combination or a composition comprising an anti-scarring drug combination, wherein the drug combination inhibits scarring between the device and a host into which the device is implanted; a method for making a medical device comprising: combining a sacral nerve stimulator for treating a bladder control problem (i.e., an electrical device) and an anti-scarring drug combination or a composition comprising an anti-scarring drug combination, wherein the drug combination inhibits scarring between the device and a host into which the device is implanted; a method for making a medical device comprising: combining a gastric nerve stimulator for treating a gastrointestinal disorder (i.e., an electrical device) and an anti-scarring drug combination or a composition comprising an anti-scarring drug combination, wherein the drug combination inhibits scarring between the device and a host into which the device is implanted; method for making a medical device comprising: combining a cochlear implant for treating deafness (i.e., an electrical device) and an anti-scarring drug combination or a composition comprising an anti-scarring drug combination, wherein the drug combination inhibits scarring between the device and a host into which the device is implanted; a method for making a medical device comprising: combining a bone growth stimulator (i.e., an electrical device) and an anti-scarring drug combination or a composition comprising an anti-scarring drug combination, wherein the drug combination inhibits scarring between the device and a host into which the device is implanted; a method for making a medical device comprising: combining a cardiac pacemaker (i.e., an electrical device) and an anti-scarring drug combination or a composition comprising an anti-scarring drug combination, wherein the drug combination inhibits scarring between the device and a host into which the device is implanted; a method for making a medical device comprising: combining an implantable cardioverter defibrillator (ICD) system (i.e., an electrical device) and an anti-scarring drug combination or a composition comprising an anti-scarring drug combination, wherein the drug combination inhibits scarring between the device and a host into which the device is implanted; a method for making a medical device comprising: combining a vagus nerve stimulator for treating arrhythmia (i.e., an electrical device) and an anti-scarring drug combination or a composition comprising an anti-scarring drug combination, wherein the drug combination inhibits scarring between the device and a host into which the device is implanted; a method for making a medical device comprising: combining an electrical lead (i.e., an electrical device) and an anti-scarring drug combination or a composition comprising an anti-scarring drug combination, wherein the drug combination inhibits scarring between the device and a host into which the device is implanted; a method for making a medical device comprising: combining a neurostimulator (i.e., an electrical device) and an anti-scarring drug combination or a composition comprising an anti-scarring drug combination, wherein the drug combination inhibits scarring between the device and a host into which the device is implanted; and a method for making a medical device comprising: combining a cardiac rhythm management device (i.e., an electrical device) and an anti-scarring drug combination or a composition comprising an anti-scarring drug combination, wherein the drug combination inhibits scarring between the device and a host into which the device is implanted.

In certain independent aspects, the present invention provides a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a neurostimulator for treating chronic pain; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a neurostimulator for treating Parkinson's Disease; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a vagal nerve stimulator for treating epilepsy; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a vagal nerve stimulator; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a sacral nerve stimulator for treating a bladder control problem; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a gastric nerve stimulator for treating a gastrointestinal disorder; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a cochlear implant for treating deafness; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a bone growth stimulator; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a cardiac pacemaker; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is an implantable cardioverter defibrillator (ICD) system; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a vagus nerve stimulator for treating arrhythmia; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is an electrical lead; a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a neurostimulator; and a method for implanting an electrical device comprising: (a) infiltrating a tissue of a host where the electrical device is to be, or has been, implanted with an anti-scarring drug combination or a composition comprising an anti-scarring drug combination and (b) implanting the electrical device into the host, wherein the electrical device is a cardiac rhythm management device.

In certain independent aspects, the present invention provides a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a neurostimulator for treating chronic pain; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a neurostimulator for treating Parkinson's Disease; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a vagal nerve stimulator for treating epilepsy; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a vagal nerve stimulator; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a sacral nerve stimulator for treating a bladder control problem; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a gastric nerve stimulator for treating a gastrointestinal disorder; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a cochlear implant for treating deafness; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a bone growth stimulator; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a cardiac pacemaker; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is an implantable cardioverter defibrillator (ICD) system; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a vagus nerve stimulator for treating arrhythmia; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is an electrical lead; a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a neurostimulator; and a method for implanting an electrical device comprising: (a) infiltrating a tissue of a host where the electrical device is to be, or has been, implanted with a first compound or a composition comprising a first compound, and (b) implanting the electrical device that comprises a second compound into the host, wherein the first and second compounds form an anti-scarring drug combination, and wherein the electrical device is a cardiac rhythm management device.

Exemplary anti-fibrotic (or anti-gliotic) drug combinations include, but are not limited to amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

Additional exemplary anti-fibrotic drug combinations include, but are not limited to, (1) a triazole (e.g., fluconazole or itraconazole) and (2) a aminopyridine (e.g., phenazopyridine (PZP), phenothiazine, dacarbazine, phenelzine); (1) an antiprotozoal (e.g., pentamidine) and (2) a diaminopyridine (e.g., phenazopyridine) or a quaternary ammonium compound (e.g., pentolinium); (1) an aromatic diamidine and (2) an antiestrogen, an anti-fungal imidazole, disulfuram, or ribavirin; (1) an aminopyridine and (2) phenothiazine, dacarbazine, or phenelzine; (1) a quaternary ammonium compound and (2) an anti-fungal imidazole, haloprogin, MnSO₄, or ZnCl₂; (1) an antiestrogen and (2) phenothiazine, cupric chloride, dacarbazine, methoxsalen, or phenelzine; (1) an antifungal imidazone and (2) disulfuram or ribavirin; (1) an estrogenic compound and dacarbazine; (1) amphotericin B and (2) dithiocarbamoyl disulfide (e.g., disulfuram); (1) terbinafine and (2) a manganese compound; (1) a tricyclic antidepressant (TCA) (e.g., amoxapine) and (2) a corticosteroid (e.g., prednisolone, glucocorticoid, mineralocorticoid); (1) a tetra-substituted pyrimidopyrimidine (e.g., dipyridamole) and (2) a corticosteroid (e.g., fludrocortisone or prednisolone); (1) a prostaglandin (e.g., alprostadil) and (2) a retinoid (e.g., tretinoin (vitamin A)); (1) an azole (e.g., imidazone or triazole) and (2) a steroid (e.g., corticosteroids including glucocorticoid or mineralocorticoid); (1) a steroid and (2) a prostaglandin, beta-adrenergic receptor ligand, anti-mitotic agent, or microtubule inhibitor; (1) a serotonin norepinephrine reuptake inhibitor (SNRI) or naradrenaline reuptake inhibitor (NARI) and (2) a corticosteroid; (1) a non-steroidal immunophilin-dependent immunosuppressant (NSIDI) (e.g., calcineurin inhibitor including cyclosporin, tacrolimus, ascomycin, pimecrolimus, ISAtx 247) and (2) a non-steroidal immunophilin-dependent immunosuppressant enhancer (NSIDIE) (e.g., selective serotonin reuptake inhibitors, tricyclic antidepressants, phenoxy phenols, anti-histamine, phenothiazines, or mu opioid receptor agonists); (1) an antihistamines and (2) an additional agent selected from corticosteroids, tricyclic or tetracyclic antidepressants, selective serotonin reuptake inhibitors, and steroid receptor modulators; (1) a tricyclic compound and (2) a corticosteroid; (1) an antipsychotic drug (e.g., chlorpromazine) and (2) an antiprotozoal drug (e.g., pentamidine); (1) an antihelmintic drug (e.g., benzimidazole) and (2) an antiprotozoal drug (e.g., pentamidine); (1) ciclopirox and (2) an antiproliferative agent; (1) a salicylanilide (e.g., niclosamide) and (2) an antiproliferative agents; (1) pentamidine or its analogue and (2) chlorpromazine or its analogue; (1) an antihelmintic drug (e.g., alberdazole, mebendazole, oxibendazole) and (2) an antiprotozoal drug (e.g., pentamidine); (1) a dibucaine or amide local anaesthetic related to bupivacaine and (2) a vinca alkaloid; (1) pentamidine, analogue or metabolite thereof and (2) an antiproliferative agent; (1) a triazole (e.g., itraconazole) and (2) an antiarrhythmic agents (e.g., amiodarone, nicardipine or bepridil); (1) an azole and (2) an HMG-CoA reductase inhibitor; a phenothiazine conjugate (e.g., a conjugate of phenothiazine and an antiproliferative agent; (1) phenothiazine and (2) an antiproliferative agent; (1) a kinesin inhibitor (e.g., phenothiazine, analog or metabolite) and (2) an antiproliferative agent (e.g., Group A and Group B antiproliferative agents); and (1) an agent that reduces the biological activity of a mitotic kinesin (e.g., chlorpromazine) and (2) an agent that reduces the biological activity of protein tyrosine phosphatase.

Additional exemplary drug combinations may comprise: (1) an anti-inflammatory agent (e.g., steroids) and (2) an agent selected from an antidepressant, an SSRI, a cardiovascular agent (e.g., an antiplatelet agent), an anti-fungal agent, and a prostaglandin; (1) a cardiovascular drug and (2) an antidepressant; (1) a cardiovascular drug and (2) a phosphodiesterase IV inhibitor; (1) an antidepressant and (2) an antihistamine; (1) an anti-fungal agent and (2) an HMG-CoA reductase inhibitor; and (1) an anti-fungal agent and (2) a metal ion (e.g., a manganese ion).

These and other agents are described in more detail herein.

These and other aspects of the present invention will become evident upon reference to the following detailed description and attached drawings. In addition, various references are set forth herein which describe in more detail certain procedures and/or compositions (e.g., polymers), and are therefore incorporated by reference in their entirety.

### BRIEF DESCRIPTION OF THE DRAWINGS

FIG. 1 is a picture that shows an uninjured carotid artery from a rat balloon injury model.

FIG. 2 is a picture that shows an injured carotid artery from a rat balloon injury model.

FIG. 3 is a picture that shows a paclitaxel/mesh treated carotid artery in a rat balloon injury model.

FIG. 4A schematically depicts the transcriptional regulation of matrix metalloproteinases.

FIG. 4B is a blot which demonstrates that IL-1 stimulates AP-1 transcriptional activity.

FIG. 4C is a graph which shows that IL-1 induced binding activity decreased in lysates from chondrocytes which were pretreated with paclitaxel.

FIG. 4D is a blot which shows that IL-1 induction increases collagenase and stromelysin in RNA levels in chondrocytes, and that this induction can be inhibited by pretreatment with paclitaxel.

FIGS. 5A-H are blots that show the effect of various anti-microtubule agents in inhibiting collagenase expression.

FIG. 6 is a graph showing the results of a screening assay for assessing the effect of paclitaxel on smooth muscle cell migration.

FIG. 7 is a bar graph showing the area of granulation tissue in carotid arteries exposed to silk-coated perivascular polyurethane (PU) films relative to arteries exposed to uncoated PU films.

FIG. 8 is a bar graph showing the area of granulation tissue in carotid arteries exposed to silk suture coated perivascular PU films relative to arteries exposed to uncoated PU films.

FIG. 9 is a bar graph showing the area of granulation tissue in carotid arteries exposed to natural and purified silk powder and wrapped with perivascular PU film relative to a control group in which arteries are wrapped with perivascular PU film only.

FIG. 10 is a bar graph showing the area of granulation tissue (at 1 month and 3 months) in carotid arteries sprinkled with talcum powder and wrapped with perivascular PU film relative to a control group in which arteries are wrapped with perivascular PU film only.

### DETAILED DESCRIPTION OF THE INVENTION

Definitions

Prior to setting forth the invention, it may be helpful to an understanding thereof to first set forth definitions of certain terms that are used hereinafter.

“Medical device”, “device”, “the device”, “medical implant”, “implant”, “medical device or implant”, “implant/device” and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing or replacing or augmenting etc. damaged or diseased organs and tissues. While medical devices are normally composed of biologically compatible synthetic materials (e.g., medical-grade stainless steel, titanium and other metals; exogenous polymers, such as polyurethane, silicon, PLA, PLGA), other materials may also be used in the construction of the medical device or implant. Specific medical devices and implants that are particularly useful for the practice of this invention include devices and implants that are used to provide electrical stimulation to the central and peripheral nervous system (including the autonomic system), cardiac muscle tissue (including myocardial conduction pathways), smooth muscle tissue and skeletal muscle tissue.

“Electrical device” refers to a medical device having electrical components that can be placed in contact with tissue in an animal host and can provide electrical excitation to nervous or muscular tissue. Electrical devices can generate electrical impulses and may be used to treat many bodily dysfunctions and disorders by blocking, masking, or stimulating electrical signals within the body. Electrical devices may comprise electrical leads and/or electrodes. Electrical medical devices of particular utility in the present invention include, but are not restricted to, devices used in the treatment of cardiac rhythm abnormalities, pain relief, epilepsy, Parkinson's Disease, movement disorders, obesity, depression, anxiety and hearing loss.

“Neurostimulator” or “neurostimulation device” refers to an electrical device for electrical excitation of the central, autonomic, or peripheral nervous system. The neurostimulator sends electrical impulses to an organ or tissue. The neurostimulator may include electrical leads as part of the electrical stimulation system. Neurostimulation may be used to block, mask, or stimulate electrical signals in the body to treat dysfunctions, including, without limitation, pain, seizures, anxiety disorders, depression, ulcers, deep vein thrombosis, muscular atrophy, obesity, joint stiffness, muscle spasms, osteoporosis, scoliosis, spinal disc degeneration, spinal cord injury, deafness, urinary dysfunction and gastroparesis. Neurostimulation may be delivered to many different parts of the nervous system, including, spinal cord, brain, vagus nerve, sacral nerve, gastric nerve, auditory nerves, as well as organs, bone, muscles and tissues. As such, neurostimulators are developed to conform to the different anatomical structures and nervous system characteristics.

“Cardiac stimulation device” or “cardiac rhythm management device” or “cardiac pacemaker” or “implantable cardiac defibrillator (ICD)” all refer to an electrical device for electrical excitation of cardiac muscle tissue (including the specialized cardiac muscle cells that make up the conductive pathways of the heart). The cardiac pacemaker sends electrical impulses to the muscle (myocardium) or conduction tissue of the heart. The pacemaker may include electrical leads as part of the electrical stimulation system. Cardiac pacemakers may be used to block, mask, or stimulate electrical signals in the heart to treat dysfunctions, including, without limitation, atrial rhythm abnormalities, conduction abnormalities and ventricular rhythm abnormalities.

“Electrical lead” refers to an electrical device that is used as a conductor to carry electrical signals from the generator to the tissues. Typically, electrical leads are composed of a connector assembly, a lead body (i.e., conductor) and an electrode. The electrical lead may be a wire or other material that transmits electrical impulses from a generator (e.g., pacemaker, defibrillator, or other neurostimulator). Electrical leads may be unipolar, in which they are adapted to provide effective therapy with only one electrode. Multi-polar leads are also available, including bipolar, tripolar and quadripolar leads.

“Fibrosis” or “scarring” refers to the formation of fibrous (scar) tissue (or in the case of injury in the CNS—the formation of glial tissue, or “gliosis”, by astrocytes) in response to injury or medical intervention.

“Inhibit fibrosis”, “reduce fibrosis”, “inhibit gliosis”, “reduce gliosis” and the like are used synonymously to refer to the action of agents or compositions which result in a statistically significant decrease in the formation of fibrous or glial tissue that may be expected to occur in the absence of the agent or composition.

Therapeutic agents which inhibit fibrosis or scarring (referred to as “anti-fibrotic agents,” “anti-fibrosis agents,” “anti-scarring agents,” “fibrosis-inhibiting agents,” or the like) can do so through one or more mechanisms including: inhibiting angiogenesis, inhibiting migration or proliferation of connective tissue cells (such as fibroblasts, smooth muscle cells, vascular smooth muscle cells), reducing extracellular matrix (ECM) production or promoting ECM breakdown, and/or inhibiting tissue remodeling. Therapeutic agents which inhibit gliosis or scarring resulting therefrom (referred to as “anti-gliosis agents,” “anti-gliotic agents,” “gliosis-inhibiting agents,” or the like) can do so through one or more mechanisms including: inhibiting migration of glial cells, inhibition of hypertrophy of glial cells, and/or inhibiting proliferation of glial cells. In addition, numerous therapeutic agents described in this invention may have the additional benefit of also reducing tissue regeneration (the replacement of injured cells by cells of the same type) when appropriate.

“Anti-scarring drug combination” (used interchangeably with “fibrosis-inhibiting drug combination,” “anti-fibrosis drug combination,” “anti-fibrotic drug combination,” “gliosis-inhibiting drug combination”, anti-gliosis drug combination”, “anti-gliotic drug combination”, or the like) refers to a combination or conjugate of two or more therapeutic agents (also referred to as “individual components”), wherein the combination or conjugate inhibits fibrosis or gliosis or scarring. Such therapeutic agents (i.e., individual components) either have anti-fibrosis (or anti-gliosis) activities themselves, or enhance anti-fibrosis (or anti-gliosis) activities of other agents in the drug combinations. In certain embodiments, each of the therapeutic agents of an anti-scarring drug combination has anti-fibrosis or anti-gliosis activities. In certain embodiments, one or more therapeutic agent(s) of an anti-scarring drug combination enhance the anti-fibrosis or anti-gliosis activities of the other therapeutic agent(s) of the combination. In certain embodiments, one or more therapeutic agent(s) of an anti-scarring drug combination, when combined with the other therapeutic agent(s), produce synergistic anti-fibrosis or anti-gliosis effects.

The compositions of the present invention may further comprise other pharmaceutically active agents. Such “other pharmaceutically active agents” (also referred to as “other biologically active agents,” or “secondary agents”) refers to agents that do not have anti-scarring activities or enhance the anti-scarring activities of another agent, but are beneficial to be used in conjunction with an anti-scarring drug combination under certain circumstances. Those agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosphate dehydrogenase) inhibitors, tyrosine kinase inhibitors, MMP inhibitors, p38 MAP kinase inhibitors, immunosuppressants, apoptosis antagonists, caspase inhibitors, cell cycle inhibitors and JNK inhibitors.

“Host”, “person”, “subject”, “patient” and the like are used synonymously to refer to the living being (human or animal) into which a device of the present invention is implanted.

“Implanted” refers to having completely or partially placed a device within a host. A device is partially implanted when some of the device reaches, or extends, to the outside of a host.

“Anti-infective agent” refers to an agent or composition which prevents microrganisms from growing and/or slows the growth rate of microorganisms and/or is directly toxic to microorganisms at or near the site of the agent. These processes would be expected to occur at a statistically significant level at or near the site of the agent or composition relative to the effect in the absence of the agent or composition.

“Inhibit infection” refers to the ability of an agent or composition to prevent microorganisms from accumulating and/or proliferating near or at the site of the agent. These processes would be expected to occur at a statistically significant level at or near the site of the agent or composition relative to the effect in the absence of the agent or composition.

“Inhibitor” refers to an agent which prevents a biological process from occurring or slows the rate or degree of occurrence of a biological process. The process may be a general one such as scarring or refer to a specific biological action such as, for example, a molecular process resulting in release of a cytokine.

“Antagonist” refers to an agent which prevents a biological process from occurring or slows the rate or degree of occurrence of a biological process. While the process may be a general one, typically this refers to a drug mechanism where the drug competes with a molecule for an active molecular site or prevents a molecule from interacting with the molecular site. In these situations, the effect is that the molecular process is inhibited.

“Agonist” refers to an agent which stimulates a biological process or rate or degree of occurrence of a biological process. The process may be a general one such as scarring or refer to a specific biological action such as, for example, a molecular process resulting in release of a cytokine.

“Anti-microtubule agents” should be understood to include any protein, peptide, chemical, or other molecule which impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett. 79(2):213-219, 1994) and Mooberry et al., (Cancer Lett. 96(2):261-266, 1995).

“Release of an agent from an implant/device” refers to a statistically significant presence of the agent, or a subcomponent thereof, which has disassociated from the implant/device.

“Biodegradable” refers to materials for which the degradation process is at least partially mediated by, and/or performed in, a biological system. “Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or pronation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

“Hyaluronic acid” or “HA” as used herein refers to all forms of hyaluronic acid that are described or referenced herein, including those that have been processed or chemically or physically modified, as well as hyaluronic acid that has been crosslinked (for example, covalently, ionically, thermally or physically). HA is a glycosaminoglycan composed of a linear chain of about 2500 repeating disaccharide units. Each disaccharide unit is composed of an N-acetylglucosamine residue linked to a glucuronic acid. Hyaluronic acid is a natural substance that is found in the extracellular matrix of many tissues including synovial joint fluid, the vitreous humor of the eye, cartilage, blood vessels, skin and the umbilical cord. Commercial forms of hyaluronic acid having a molecular weight of approximately 1.2 to 1.5 million Daltons (Da) are extracted from rooster combs and other animal sources. Other sources of HA include HA that is isolated from cell culture/fermentation processes. Lower molecular weight HA formulations are also available from a variety of commercial sources. The molecule can be of variable lengths (i.e., different numbers of repeating disaccharide units and different chain branching patterns) and can be modified at several sites (through the addition or subtraction of different functional groups) without deviating from the scope of the present invention.

The term “inter-react” refers to the formulation of covalent bonds, noncovalent bonds, or both. The term thus includes crosslinking, which involves both intermolecular crosslinks and optionally intramolecular crosslinks as well, arising from the formation of covalent bonds. Covalent bonding between two reactive groups may be direct, in which case an atom in reactive group is directly bound to an atom in the other reactive group, or it may be indirect, through a linking group. Noncovalent bonds include ionic (electrostatic) bonds, hydrogen bonds, or the association of hydrophobic molecular segments, which may be the same or different. A crosslinked matrix may, in addition to covalent bonds, also include such intermolecular and/or intramolecular noncovalent bonds.

When referring to polymers, the terms “hydrophilic” and “hydrophobic” are generally defined in terms of an HLB value, i.e., a hydrophilic lipophilic balance. A high HLB value indicates a hydrophilic compound, while a low HLB value characterizes a hydrophobic compound. HLB values are well known in the art, and generally range from 1 to 18. Preferred multifunctional compound cores are hydrophilic, although as long as the multifunctional compound as a whole contains at least one hydrophilic component, crosslinkable hydrophobic components may also be present.

The term “synthetic” is used to refer to polymers, compounds and other such materials that are “chemically synthesized.” For example, a synthetic material in the present compositions may have a molecular structure that is identical to a naturally occurring material, but the material per se, as incorporated in the compositions of the invention, has been chemically synthesized in the laboratory or industrially. “Synthetic” materials also include semi-synthetic materials, i.e., naturally occurring materials, obtained from a natural source, that have been chemically modified in some way. Generally, however, the synthetic materials herein are purely synthetic, i.e., they are neither semi-synthetic nor have a structure that is identical to that of a naturally occurring material.

The term “effective amount” refers to the amount of composition required in order to obtain the effect desired. For example, an “effective amount for inhibiting fibosis” of a composition refers to the amount needed to inhibit fibrosis to a detectable degree. The actual amount that is determined to be an effective amount will vary depending on factors such as the size, condition, sex and age of the patient and can be more readily determined by the caregiver.

The term “in situ” as used herein means at the site of administration. Thus, compositions of the invention can be injected or otherwise applied to a specific site within a patient's body, e.g., a site in need of augmentation, and allowed to crosslink at the site of injection. Suitable sites will generally be intradermal or subcutaneous regions for augmenting dermal support, at a bone fracture site for bone repair, within sphincter tissue for sphincter augmentation (e.g., for restoration of continence), within a wound or suture, to promote tissue regrowth; and within or adjacent to vessel anastomoses, to promote vessel regrowth.

The term “aqueous medium” includes solutions, suspensions, dispersions, colloids, and the like containing water. The term “aqueous environment” means an environment containing an aqueous medium. Similarly, the term “dry environment” means an environment that does not contain an aqueous medium.

With regard to nomenclature pertinent to molecular structures, the following definitions apply:

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups. The C₁₋₇ alkyl group may be substituted or unsubstituted. C₁₋₇ alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

The term “lower alkyl” intends an alkyl group of one to six carbon atoms, preferably one to four carbon atoms.

“Substituted alkyl” refers to alkyl substituted with one or more substitutent groups.

“Alkylene,” “lower alkylene” and “substituted alkylene” refer to divalent alkyl, lower alkyl, and substituted alkyl groups, respectively.

The term “aryl” as used herein, and unless otherwise specified, refers to an aromatic substitutent containing a single aromatic ring (monocyclic) or multiple aromatic rings that are fused together, linked covalently, or linked to a common group such as a methylene or ethylene moiety. The common linking group may also be a carbonyl as in benzophenone, an oxygen atom as in diphenylether, or a nitrogen atom as in diphenylamine. Preferred aryl groups contain one aromatic ring or two fused or linked aromatic rings, e.g., phenyl, naphthyl, biphenyl, diphenylether, diphenylamine, benzophenone, and the like.

“Substituted aryl” refers to an aryl moiety substituted with one or more substitutent groups.

The terms “heteroatom-containing aryl” and “heteroaryl” refer to aryl in which at least one carbon atom is replaced with a heteroatom. The terms “arylene” and “substituted arylene” refer to divalent aryl and substituted aryl groups as just defined.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon.

“Hydrocarbyl” refers to univalent hydrocarbyl radicals containing 1 to about 30 carbon atoms, preferably 1 to about 24 carbon atoms, most preferably 1 to about 12 carbon atoms, including branched or unbranched, saturated or unsaturated species, such as alkyl groups, alkenyl groups, aryl groups, and the like. The term “lower hydrocarbyl” intends a hydrocarbyl group of one to six carbon atoms, preferably one to four carbon atoms. The term “hydrocarbylene” intends a divalent hydrocarbyl moiety containing 1 to about 30 carbon atoms, preferably 1 to about 24 carbon atoms, most preferably 1 to about 12 carbon atoms, including branched or unbranched, saturated or unsaturated species, or the like. The term “lower hydrocarbylene” intends a hydrocarbylene group of one to six carbon atoms, preferably one to four carbon atoms. “Substituted hydrocarbyl” refers to hydrocarbyl substituted with one or more substitutent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substitutent groups, and the terms “heteroatom-containing hydrocarbylene” and “heterohydrocarbylene” refer to hydrocarbylene in which at least one carbon atom is replaced with a heteroatom. If not otherwise indicated, “hydrocarbyl” indicates both unsubstituted and substituted hydrocarbyls, “heteroatom-containing hydrocarbyl” indicates both unsubstituted and substituted heteroatom-containing hydrocarbyls and so forth.

By “C₂₋₇ alkenyl” is meant a branched or unbranched hydrocarbon group containing one or more double bonds and having from 2 to 7 carbon atoms. A C₂₋₇ alkenyl may optionally include monocyclic or polycyclic rings, in which each ring desirably has from three to six members. The C₂₋₇ alkenyl group may be substituted or unsubstituted. C₂₋₇ alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl.

By “C₂₋₇ alkynyl” is meant a branched or unbranched hydrocarbon group containing one or more triple bonds and having from 2 to 7 carbon atoms. A C₂₋₇ alkynyl may optionally include monocyclic, bicyclic, or tricyclic rings, in which each ring desirably has five or six members. The C₂₋₇ alkynyl group may be substituted or unsubstituted. C₂₋₇ alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; and 3-methyl-4-pentynyl.

By “C₂₋₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from the group consisting of N, O, and S and including any bicyclic group in which any of the above-defined heterocyclic rings is fused to a benzene ring. The heterocyclyl group may be substituted or unsubstituted. The nitrogen and sulfur heteroatoms may optionally be oxidized. The heterocyclic ring may be covalently attached via any heteroatom or carbon atom that results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 1,4,5,6-tetrahydro pyridinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, 1,4,5,6-tetrahydro pyridinyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 7 to 14 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substitutents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups.

By “alkoxy” is meant a chemical substitutent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substitutent of the formula —OR, wherein R is a C₆₋₁₂ aryl group.

By “amido” is meant a chemical substitutent of the formula —NRR′, wherein the nitrogen atom is part of an amide bond (e.g., —C(O)—NRR′) and wherein R and R′ are each, independently, selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl, or —NRR′ forms a C₂₋₆ heterocyclyl ring, as defined above, but containing at least one nitrogen atom, such as piperidino, morpholino, and azabicyclo, among others.

By “fluoroalkyl” is meant an alkyl group that is substituted with a fluorine.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkylthio” is meant a chemical substitutent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substitutent of the formula —SR, wherein R is a C₆₋₁₂ aryl group.

By “quaternary amino” is meant a chemical substitutent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substitutent, to another moiety. The nitrogen atom, N, is covalently attached to four carbon atoms of alkyl and/or aryl groups, resulting in a positive charge at the nitrogen atom.

By “carbo(C₁-C₆ alkoxy)” is meant an ester fragment of the structure CO₂R, wherein R is an alkyl group.

By “carbo(C₆-C₁₈ aryl-C₁-C₆ alkoxy)” is meant an ester fragment of the structure CO₂R, wherein R is an alkaryl group.

By “aryl” is meant a C₆-C₁₈ carbocyclic aromatic ring or ring system. Examples of aryl groups include phenyl, naphthyl, biphenyl, fluorenyl, and indenyl groups. The term “heteroaryl” means a C₁-C₉ aromatic ring or ring systems that contains at least one ring heteroatom (e.g., O, S, N). Heteroaryl groups include furyl, thienyl, pyridyl, quinolinyl, tetrazolyl, and imidazolyl groups.

By “halide” or “halogen” is meant bromine, chlorine, iodine, or fluorine.

By “heterocycle” is meant a C₁-C₉ non-aromatic ring or ring system that contains at least one ring heteroatom (e.g., O, S, N). Heterocycles include, for example, pyrrolidinyl, tetrahydrofuranyl, morpholinyl, thiazolidinyl, and imidazolidinyl groups.

Aryl, hetero, and heterocycle groups may be unsubstituted or substituted by one or more substitutents selected from the group consisting of C₁₋₆ alkyl, hydroxy, halo, nitro, C₁₋₆ alkoxy, C₁₋₆ alkylthio, trihalomethyl, C₁₋₆ acyl, carbonyl, heteroarylcarbonyl, nitrile, C₁₋₆ alkoxycarbonyl, oxo, alkyl (wherein the alkyl group has from 1 to 6 carbon atoms) and heteroarylalkyl (wherein the alkyl group has from 1 to 6 carbon atoms).

By “aromatic residue” is meant an aromatic group having a ring system with conjugated π electrons (e.g., phenyl, or imidazole). The ring of the aryl group is preferably to 10 atoms. The aromatic ring may be exclusively composed of carbon atoms or may be composed of a mixture of carbon atoms and heteroatoms. Preferred heteroatoms include nitrogen, oxygen, sulfur, and phosphorous. Aryl groups may optionally include monocyclic, bicyclic, or tricyclic rings, where each ring has preferably five or six members. The aryl group may be substituted or unsubstituted. Exemplary substitutents include alkyl, hydroxyl, alkoxy, aryloxy, sulfhydryl, alkylthio, arylthio, halogen, fluoroalkyl, carboxyl, carboxyalkyl, amino, aminoalkyl, monosubstituted amino, disubstituted amino, and quaternary amino groups.

By “non-vicinal O, S, or N” is meant an oxygen, sulfur, or substituted or unsubstituted nitrogen heteroatom substitutent in a linkage, wherein the heteroatom substitutent does not form a bond to a saturated carbon that is bonded to another heteroatom.

The term “substituted” as used herein means any of the above groups (e.g., alkyl, alkoxy, acyl, aryl, heteroaryl and heterocycle) wherein at least one hydrogen atom is replaced with a substitutent. In the case of an oxo substitutent (“═O”) two hydrogen atoms are replaced. Substituents include halogen, hydroxy, oxo, alkyl, aryl, alkoxy, aryloxy, acyl, mercapto, cyano, alkylthio, arylthio, heteroarylthio, heteroaryl, heterocycle, —NRₐR_{b}, —NRₐC(═O)R_{b}, —NR_{c}C(═O)NRₐR_{b}, —NRₐC(═O)OR_{b}, —NRₐSO₂R_{b}, —C(═O)NRₐR_{b}, —OC(═O)Rₐ, —OC(═O)ORₐ, —OC(═O)NRₐR_{b}, —NRₐSO₂R_{b} or a radical of the formula —Y-Z-Rₐ where Y is alkanediyl, substituted alkanediyl or a direct bond, alkanediyl refers to a divalent alkyl with two hydrogen atoms taken from the same or different carbon atoms, Z is —O—, —S—, —S(═O)—, —S(═O)₂—, —N(R_{b})—, —C(═O)—, —C(═O)O—, —OC(═O)—, —N(R_{b})C(═O)—, —C(═O)N(R_{b})— or a direct bond, wherein Rₐ, R_{b} and R_{c} are the same or different and independently hydrogen, amino, alkyl, substituted alkyl (including halogenated alkyl), aryl, substituted aryl, heteroaryl, substituted heteroaryl, heterocycle or substituted heterocycle or wherein Rₐ and R_{b} taken together with the nitrogen atom to which they are attached form a heterocycle or substituted heterocycle.

Unless otherwise indicated, it is to be understood that specified molecular segments can be substituted with one or more substitutents that do not compromise a compound's utility. For example, “succinimidyl” is intended to include unsubstituted succinimidyl as well as sulfosuccinimidyl and other succinimidyl groups substituted on a ring carbon atom, e.g., with alkoxy substitutents, polyether substitutents, or the like.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. As used herein, the term “about” refers to +15% of any indicated structure, value, or range.

“A” and “an” refer to one or more of the indicated items. For example, “a” polymer refers to both one polymer and a mixture comprising two or more polymers; “a multifunctional compound” refers not only to a single multifunctional compound but also to a combination of two or more of the same or different multifunctional compounds; “a reactive group” refers to a combination of reactive groups as well as to a single reactive group, and the like.

As discussed above, the present invention provides compositions, methods and devices relating to medical devices and implants, which greatly increase their ability to inhibit the formation of reactive fibrotic (or glial) tissue on, or around, the surface of the device or implant. Described in more detail below are methods for constructing medical devices or implants, compositions and methods for generating medical devices and implants which inhibit fibrosis (or gliosis), and methods for utilizing such medical devices and implants.

Clinical Applications of Electrical Medical Devices and Implants which Contain a Fibrosis-Inhibiting (or Gliosis-Inhibiting) Agent

Medical devices having electrical components, such as electrical pacing or stimulating devices, can be implanted in the body to provide electrical conduction to the central and peripheral nervous system (including the autonomic system), cardiac muscle tissue (including myocardial conduction pathways), smooth muscle tissue and skeletal muscle tissue. These electrical impulses are used to treat many bodily dysfunctions and disorders by blocking, masking, stimulating, or replacing electrical signals within the body. Examples include pacemaker leads used to maintain the normal rhythmic beating of the heart; defibrillator leads used to “re-start” the heart when it stops beating; peripheral nerve stimulating devices to treat chronic pain; deep brain electrical stimulation to treat conditions such as tremor, Parkinson's disease, movement disorders, epilepsy, depression and psychiatric disorders; and vagal nerve stimulation to treat epilepsy, depression, anxiety, obesity, migraine and Alzheimer's Disease.

The clinical function of an electrical device such as a cardiac pacemaker lead, neurostimulation lead, or other electrical lead depends upon the device being able to effectively maintain intimate anatomical contact with the target tissue (typically electrically excitable cells such as muscle or nerve) such that electrical conduction from the device to the tissue can occur. Unfortunately, in many instances when these devices are implanted in the body, they are subject to a “foreign body” response from the surrounding host tissues. The body recognizes the implanted device as foreign, which triggers an inflammatory response followed by encapsulation of the implant with fibrous connective tissue (or glial tissue—called “gliosis”—when it occurs within the central nervous system). Scarring (i.e., fibrosis or gliosis) can also result from trauma to the anatomical structures and tissue surrounding the implant during the implantation of the device. Lastly, fibrous encapsulation of the device can occur even after a successful implantation if the device is manipulated (some patients continuously “fiddle” with a subcutaneous implant) or irritated by the daily activities of the patient. When scarring occurs around the implanted device, the electrical characteristics of the electrode-tissue interface degrade, and the device may fail to function properly. For example, it may require additional electrical current from the lead to overcome the extra resistance imposed by the intervening fibrotic (or glial) tissue. This can shorten the battery life of an implant (making more frequent removal and re-implantation necessary), prevent electrical conduction altogether (rendering the implant clinically ineffective) and/or cause damage to the target tissue. Additionally, the surrounding tissue may be inadvertently damaged from the inflammatory foreign body response, which can result in loss of function or tissue necrosis.

The present invention addresses these problems. Exemplary electrical devices are described below.

1) Neurostimulation Devices

In one aspect, the electrical device may be a neurostimulation device where a pulse generator delivers an electrical impulse to a nervous tissue (e.g., CNS, peripheral nerves, autonomic nerves) in order to regulate its activity. There are numerous neurostimulator devices where the occurrence of a fibrotic reaction may adversely affect the functioning of the device or the biological problem for which the device was implanted or used. Typically, fibrotic encapsulation of the electrical lead (or the growth of fibrous tissue between the lead and the target nerve tissue) slows, impairs, or interrupts electrical transmission of the impulse from the device to the tissue. This can cause the device to function suboptimally or not at all, or can cause excessive drain on battery life because increased energy is required to overcome the electrical resistance imposed by the intervening scar (or glial) tissue.

Neurostimulation devices are used as alternative or adjunctive therapy for chronic, neurodegenerative diseases, which are typically treated with drug therapy, invasive therapy, or behavioral/lifestyle changes. Neurostimulation may be used to block, mask, or stimulate electrical signals in the body to treat dysfunctions, including, without limitation, pain, seizures, anxiety disorders, depression, ulcers, deep vein thrombosis, muscular atrophy, obesity, joint stiffness, muscle spasms, osteoporosis, scoliosis, spinal disc degeneration, spinal cord injury, deafness, urinary dysfunction and gastroparesis. Neurostimulation may be delivered to many different parts of the nervous system, including, spinal cord, brain, vagus nerve, sacral nerve, gastric nerve, auditory nerves, as well as organs, bone, muscles and tissues. As such, neurostimulators are developed to conform to the different anatomical structures and nervous system characteristics. Representative examples of neurologic and neurosurgical implants and devices that can be coated with, or otherwise constructed to contain and/or release the therapeutic agents provided herein, include, e.g., nerve stimulator devices to provide pain relief, devices for continuous subarachnoid infusions, implantable electrodes, stimulation electrodes, implantable pulse generators, electrical leads, stimulation catheter leads, neurostimulation systems, electrical stimulators, cochlear implants, auditory stimulators and micro stimulators.

Neurostimulation devices may also be classified based on their source of power, which includes: battery powered, radio-frequency (RF) powered, or a combination of both types. For battery powered neurostimulators, an implanted, non-rechargeable battery is used for power. The battery and leads are all surgically implanted and thus the neurostimulation device is completely internal. The settings of the totally implanted neurostimulator are controlled by the patient through an external magnet. The lifetime of the implant is generally limited by the duration of battery life and ranges from two to four years depending upon usage and power requirements. For RF-powered neurostimulation devices, the radio-frequency is transmitted from an externally worn source to an implanted passive receiver. Since the power source is readily rechargeable or replaceable, the radio-frequency system enables greater power resources and thus, multiple leads may be used in these systems. Specific examples include a neurostimulator that has a battery power source contained within to supply power over an eight hour period in which power may be replenished by an external radio frequency coupled device (See e.g., U.S. Pat. No. 5,807,397) or a microstimulator which is controlled by an external transmitter using data signals and powered by radio frequency (See e.g., U.S. Pat. No. 6,061,596).

Examples of commercially available neurostimulation products include a radio-frequency powered neurostimulator comprised of the 3272 MATTRIX Receiver, 3210 MATTRIX Transmitter and 3487A PISCES-QUAD Quadripolar Leads made by Medtronic, Inc. (Minneapolis, Minn.). Medtronic also sells a battery-powered ITREL 3 Neurostimulator and SYNERGY Neurostimulator, the INTERSIM Therapy for sacral nerve stimulation for urinary control, and leads such as the 3998 SPECIFY Lead and 3587A RESUME II Lead.

Another example of a neurostimulation device is a gastric pacemaker, in which multiple electrodes are positioned along the GI tract to deliver a phased electrical stimulation to pace peristaltic movement of the material through the GI tract. See, e.g., U.S. Pat. No. 5,690,691. A representative example of a gastric stimulation device is the ENTERRA Gastric Electrical Stimulation (GES) from Medtronic, Inc. (Minneapolis, Minn.).

The neurostimulation device, particularly the lead(s), must be positioned in a very precise manner to ensure that stimulation is delivered to the correct anatomical location in the nervous system. All, or parts, of a neurostimulation device can migrate following surgery, or excessive scar (or glial) tissue growth can occur around the implant, which can lead to a reduction in the performance of these devices (as described previously). Neurostimulator devices that release a therapeutic agent for reducing scarring (or gliosis) at the electrode-tissue interface can be used to increase the efficacy and/or the duration of activity (particularly for fully-implanted, battery-powered devices) of the implant. Accordingly, the present invention provides neurostimulator leads that are coated with an anti-scarring drug combination, or individual component(s) thereof, or a composition that includes an anti-scarring (or anti-gliosis) drug combination, or individual component(s) thereof.

For greater clarity, several specific neurostimulation devices and treatments will be described in greater detail including:

a) Neurostimulation for the Treatment of Chronic Pain

Chronic pain is one of the most important clinical problems in all of medicine. For example, it is estimated that over 5 million people in the United States are disabled by back pain. The economic cost of chronic back pain is enormous, resulting in over 100 million lost work days annually at an estimated cost of $50-100 billion. It has been reported that approximately 40 million Americans are afflicted with recurrent headaches and that the cost of medications for this condition exceeds $4 billion a year. A further 8 million people in the U.S. report that they experience chronic neck or facial pain and spend an estimated $2 billion a year for treatment. The cost of managing pain for oncology patients is thought to approach $12 billion. Chronic pain disables more people than cancer or heart disease and costs the American public more than both cancer and heart disease combined. In addition to the physical consequences, chronic pain has numerous other costs including loss of employment, marital discord, depression and prescription drug addiction. It goes without saying, therefore, that reducing the morbidity and costs associated with persistent pain remains a significant challenge for the healthcare system.

Intractable severe pain resulting from injury, illness, scoliosis, spinal disc degeneration, spinal cord injury, malignancy, arachnoiditis, chronic disease, pain syndromes (e.g., failed back syndrome, complex regional pain syndrome) and other causes is a debilitating and common medical problem. In many patients, the continued use of analgesics, particularly drugs like narcotics, are not a viable solution due to tolerance, loss of effectiveness, and addiction potential. In an effort to combat this, neurostimulation devices have been developed to treat severe intractable pain that is resistant to other traditional treatment modalities such as drug therapy, invasive therapy (surgery), or behavioral/lifestyle changes.

In principle, neurostimulation works by delivering low voltage electrical stimulation to the spinal cord or a particular peripheral nerve in order to block the sensation of pain. The Gate Control Theory of Pain (Ronald Melzack and Patrick Wall) hypothesizes that there is a “gate” in the dorsal horn of the spinal cord that controls the flow of pain signals from the peripheral receptors to the brain. It is speculated that the body can inhibit the pain signals (“close the gate”) by activating other (non-pain) fibers in the region of the dorsal horn. Neurostimulation devices are implanted in the epidural space of the spinal cord to stimulate non-noxious nerve fibers in the dorsal horn and mask the sensation of pain. As a result the patient typically experiences a tingling sensation (known as paresthesia) instead of pain. With neurostimulation, the majority of patients will report improved pain relief (50% reduction), increased activity levels and a reduction in the use of narcotics.

Pain management neurostimulation systems consist of a power source that generates the electrical stimulation, leads (typically 1 or 2) that deliver electrical stimulation to the spinal cord or targeted peripheral nerve, and an electrical connection that connects the power source to the leads. Neurostimulation systems can be battery powered, radio-frequency powered, or a combination of both. In general, there are two types of neurostimulation devices: those that are surgically implanted and are completely internal (i.e., the battery and leads are implanted), and those with internal (leads and radio-frequency receiver) and external (power source and antenna) components. For internal, battery-powered neurostimulators, an implanted, non-rechargeable battery and the leads are all surgically implanted. The settings of the totally implanted neurostimulator may be controlled by the host by using an external magnet and the implant has a lifespan of two to four years. For radio-frequency powered neurostimulators, the radio-frequency is transmitted from an externally worn source to an implanted passive receiver. The radio-frequency system enables greater power resources and thus, multiple leads may be used.

There are numerous neurostimulation devices that can be used for spinal cord stimulation in the management of pain control, postural positioning and other disorders. Examples of specific neurostimulation devices include those composed of a sensor that detects the position of the spine and a stimulator that automatically emits a series of pulses which decrease in amplitude when back is in a supine position. See e.g., U.S. Pat. Nos. 5,031,618 and 5,342,409. The neurostimulator may be composed of electrodes and a control circuit which generates pulses and rest periods based on intervals corresponding to the body's activity and regeneration period as a treatment for pain. See e.g., U.S. Pat. No. 5,354,320. The neurostimulator, which may be implanted within the epidural space parallel to the axis of the spinal cord, may transmit data to a receiver which generates a spinal cord stimulation pulse that may be delivered via a coupled, multi-electrode. See e.g., U.S. Pat. No. 6,609,031. The neurostimulator may be a stimulation catheter lead with a sheath and at least three electrodes that provide stimulation to neural tissue. See e.g., U.S. Pat. No. 6,510,347. The neurostimulator may be a self-centering epidural spinal cord lead with a pivoting region to stabilize the lead which inflates when injected with a hardening agent. See e.g., U.S. Pat. No. 6,308,103. Other neurostimulators used to induce electrical activity in the spinal cord are described in, e.g., U.S. Pat. Nos. 6,546,293; 6,236,892; 4,044,774 and 3,724,467.

Commercially available neurostimulation devices for the management of chronic pain include the SYNERGY, INTREL, X-TREL and MATTRIX neurostimulation systems from Medtronic, Inc. The percutaneous leads in this system can be quadripolar (4 electrodes), such as the PISCES-QUAD, PISCES-QUAD PLUS and the PISCES-QUAD Compact, or octapolar (8 electrodes) such as the OCTAD lead. The surgical leads themselves are quadripolar, such as the SPECIFY Lead, the RESUME II Lead, the RESUME TL Lead and the ON-POINT PNS Lead, to create multiple stimulation combinations and a broad area of paresthesia. These neurostimulation systems and associated leads may be described, for example, in U.S. Pat. Nos. 6,671,544; 6,654,642; 6,360,750; 6,353,762; 6,058,331; 5,342,409; 5,031,618 and 4,044,774. Neurostimulating leads such as these may benefit from release of a therapeutic agent able to reducing scarring at the electrode-tissue interface to increase the efficiency of impulse transmission and increase the duration that the leads function clinically. In one aspect, the device includes spinal cord stimulating devices and/or leads that are coated with an anti-scarring (or anti-gliosis) drug combination, or individual component(s) thereof, or a composition that includes an anti-scarring (or anti-gliosis) drug combination, or individual component(s) thereof. As an alternative to this, or in addition to this, a composition that includes an anti-scarring drug combination, or individual component(s) thereof, can be infiltrated into the epidural space where the lead will be implanted. Other commercially available systems that may useful for the practice of this invention as described above include the rechargeable PRECISION Spinal Cord Stimulation System (Advanced Bionics Corporation, Sylmar, Calif.; which is a Boston Scientific Company) which can drive up to 16 electrodes (see e.g., U.S. Pat. Nos. 6,735,474; 6,735,475; 6,659,968; 6,622,048; 6,516,227 and 6,052,624). The GENESIS XP Spinal Cord Stimulator available from Advanced Neuromodulation Systems, Inc. (Plano, Tex.; see e.g., U.S. Pat. Nos. 6,748,276; 6,609,031 and 5,938,690) as well as the Vagus Nerve Stimulation (VNS) Therapy System available from Cyberonics, Inc. (Houston, Tex.; see e.g., U.S. Pat. Nos. 6,721,603 and 5,330,515) may also benefit from the application of anti-fibrosis (or anti-gliosis) drug combination, or individual component(s) thereof, as described in this invention.

Regardless of the specific design features, for neurostimulation to be effective in pain relief, the leads must be accurately positioned adjacent to the portion of the spinal cord or the targeted peripheral nerve that is to be electrically stimulated. Neurostimulators can migrate following surgery or excessive tissue growth or extracellular matrix deposition can occur around neurostimulators, which can lead to a reduction in the functioning of these devices. Neurostimulator devices that release therapeutic agent for reducing scarring at the electrode-tissue interface can be used to increase the duration that these devices clinically function. In one aspect, the device includes neurostimulator devices and/or leads that are coated with an anti-scarring (or anti-gliosis) drug combination, or individual component(s) thereof, or a composition that includes an anti-scarring (or anti-gliosis) drug combination, or individual component(s) thereof. As an alternative to this, or in addition to this, a composition that includes an anti-scarring (anti-gliosis) drug combination, or individual component(s) thereof, can be infiltrated into the tissue surrounding the implanted portion (particularly the leads) of the pain management neurostimulation device.

b) Neurostimulation for the Treatment of Parkinson's Disease

Neurostimulation devices implanted into the brain are used to control the symptoms associated with Parkinson's disease or essential tremor. Typically, these are dual chambered stimulator devices (similar to cardiac pacemakers) that deliver bilateral stimulation to parts of the brain that control motor function. Electrical stimulation is used to relieve muscular symptoms due to Parkinson's disease itself (tremor, rigidity, bradykinesia, akinesia) or symptoms that arise as a result of side effects of the medications used to treat the disease (dyskinesias). Two stimulating electrodes are implanted in the brain (usually bilaterally in the subthalamic nucleus or the globus pallidus interna) for the treatment of levodopa-responsive Parkinson's and one is implanted (in the ventral intermediate nucleus of the thalamus) for the treatment of tremor. The electrodes are implanted in the brain by a functional stereotactic neurosurgeon using a stereotactic head frame and MRI or CT guidance. The electrodes are connected via extensions (which run under the skin of the scalp and neck) to a neurostimulatory (pulse generating) device implanted under the skin near the clavicle. A neurologist can then optimize symptom control by adjusting stimulation parameters using a noninvasive control device that communicates with the neurostimulator via telemetry. The patient is also able to turn the system on and off using a magnet and control the device (within limits set by the neurologist) settings using a controller device. This form of deep brain stimulation has also been investigated for the treatment pain, epilepsy, psychiatric conditions (obsessive-compulsive disorder) and dystonia.

Several devices have been described for such applications including, for example, a neurostimulator and an implantable electrode that has a flexible, non-conducting covering material, which is used for tissue monitoring and stimulation of the cortical tissue of the brain as well as other tissue. See e.g., U.S. Pat. No. 6,024,702. The neurostimulator (pulse generator) may be an intracranially implanted electrical control module and a plurality of electrodes which stimulate the brain tissue with an electrical signal at a defined frequency. See e.g., U.S. Pat. No. 6,591,138. The neurostimulator may be a system composed of at least two electrodes adapted to the cranium and a control module adapted to be implanted beneath the scalp for transmitting output electrical signals and also external equipment for providing two-way communication. See e.g., U.S. Pat. No. 6,016,449. The neurostimulator may be an implantable assembly composed of a sensor and two electrodes, which are used to modify the electrical activity in the brain. See e.g., U.S. Pat. No. 6,466,822.

A commercial example of a device used to treat Parkinson's disease and essential tremor includes the ACTIVA System by Medtronic, Inc. (see, for example, U.S. Pat. Nos. 6,671,544 and 6,654,642). This system consists of the KINETRA Dual Chamber neurostimulator, the SOLETRA neurostimulator or the INTREL neurostimulator, connected to an extension (an insulated wire), that is further connected to a DBS lead. The DBS lead consists of four thin, insulated, coiled wires bundled with polyurethane. Each of the four wires ends in a 1.5 mm long electrode. Although all or parts of the DBS lead may be suitable for coating with a fibrosis-inhibiting (or gliosis-inhibiting) drug combination, or individual component(s) thereof, a preferred embodiment involves delivering the therapeutic agent from the surface of the four electrodes. As an alternative to this, or in addition to this, a composition that includes an anti-fibrosis (or anti-gliosis) drug combination, or individual component(s) thereof, can be infiltrated into the brain tissue surrounding the leads.

c) Vagal Nerve Stimulation for the Treatment of Epilepsy

Neurostimulation devices are also used for vagal nerve stimulation in the management of pharmacoresistant epilepsy (i.e., epilepsy that is uncontrolled despite appropriate medical treatment with ant-epileptic drugs). Approximately 30% of epileptic patients continue to have seizures despite of multiple attempts at controlling the disease with drug therapy or are unable to tolerate the side effects of their medications. It is estimated that approximately 2.5 million patients in the United States suffer from treatment-resistant epilepsy and may benefit from vagal nerve stimulation therapy. As such, inadequate seizure control remains a significant medical problem with many patients suffering from diminished self esteem, poor academic achievement and a restricted lifestyle as a result of their illness.

The vagus nerve (also called the 10^{th} cranial nerve) contains primarily afferent sensory fibres that carry information from the neck, thorax and abdomen to the nucleus tractus soltarius of the brainstem and on to multiple noradrenergic and serotonergic neuromodulatory systems in the brain and spinal cord. Vagal nerve stimulation (VNS) has been shown to induce progressive EEG changes, alter bilateral cerebral blood flow, and change blood flow to the thalamus. Although the exact mechanism of seizure control is not known, VNS has been demonstrated clinically to terminate seizures after seizure onset, reduce the severity and frequency of seizures, prevent seizures when used prophylactically over time, improve quality of life, and reduce the dosage, number and side effects of anti-epileptic medications (resulting in improved alertness, mood, memory).

In VNS, a bipolar electrical lead is surgically implanted such that it transmits electrical stimulation from the pulse generator to the left vagus nerve in the neck. The pulse generator is an implanted, lithium carbon monofluoride battery-powered device that delivers a precise pattern of stimulation to the vagus nerve. The pulse generator can be programmed (using a programming wand) by the neurologist to suit an individual patient's symptoms, while the patient can turn the device on and off through the use of an external magnet. Chronic electrical stimulation which can be used as a direct treatment for epilepsy is described in, for example, U.S. Pat. No. 6,016,449, whereby, an implantable neurostimulator is coupled to relatively permanent deep brain electrodes. The implantable neurostimulator may be composed of an implantable electrical lead having a furcated, or split, distal portion with two or more separate end segments, each of which bears at least one sensing or stimulation electrode, which may be used to treat epilepsy and other neurological disorders. See e.g., U.S. Pat. No. 6,597,953.

A commercial example of a VNS system is the product produced by Cyberonics, Inc. that includes the Model 300 and Model 302 leads, the Model 101 and Model 102R pulse generators, the Model 201 programming wand and Model 250 programming software, and the Model 220 magnets. These products manufactured by Cyberonics, Inc. may be described, for example, in U.S. Pat. Nos. 5,540,730 and 5,299,569.

Regardless of the specific design features, for vagal nerve stimulation to be effective in epilepsy, the leads must be accurately positioned adjacent to the left vagus nerve. If excessive scar tissue growth or extracellular matrix deposition occurs around the VNS leads, this can reduce the efficacy of the device. VNS devices that release a therapeutic agent able to reducing scarring at the electrode-tissue interface can increase the efficiency of impulse transmission and increase the duration that these devices function clinically. In one aspect, the device includes VNS devices and/or leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be infiltrated into the tissue surrounding the vagus nerve where the lead will be implanted.

d) Vagal Nerve Stimulation for the Treatment of Other Disorders

It was discovered during the use of VNS for the treatment of epilepsy that some patients experienced an improvement in their mood during therapy. As such, VNS is currently being examined for use in the management of treatment-resistant mood disorders such as depression and anxiety. Depression remains an enormous clinical problem in the Western World with over 1% (25 million people in the United States) suffering from depression that is inadequately treated by pharmacotherapy. Vagal nerve stimulation has been examined in the management of conditions such as anxiety (panic disorder, obsessive-compulsive disorder, post-traumatic stress disorder), obesity, migraine, sleep disorders, dementia, Alzheimer's disease and other chronic or degenerative neurological disorders. VNS has also been examined for use in the treatment of medically significant obesity.

The implantable neurostimulator for the treatment of neurological disorders may be composed of an implantable electrical lead having a furcated, or split, distal portion with two or more separate end segments, each of which bears at least one sensing or stimulation electrode. See e.g., U.S. Pat. No. 6,597,953. The implantable neurostimulator may be an apparatus for treating Alzheimer's disease and dementia, particularly for neuro modulating or stimulating left vagus nerve, composed of an implantable lead-receiver, external stimulator, and primary coil. See e.g., U.S. Pat. No. 6,615,085.

Cyberonics, Inc. manufactures the commercially available VNS system, including the Model 300 and Model 302 leads, the Model 101 and Model 102R pulse generators, the Model 201 programming wand and Model 250 programming software, and the Model 220 magnets. These products as well as others that are being developed by Cyberonics, Inc. may be used to treat neurological disorders, including depression (see e.g., U.S. Pat. No. 5,299,569), dementia (see e.g., U.S. Pat. No. 5,269,303), migraines (see e.g., U.S. Pat. No. 5,215,086), sleep disorders (see e.g., U.S. Pat. No. 5,335,657) and obesity (see e.g., U.S. Pat. Nos. 6,587,719; 6,609,025; 5,263,480 and 5,188,104).

It is important to note that the fundamentals of treatment are identical to those described above for epilepsy. The devices employed and the principles of therapy are also similar. As was described above for the treatment of epilepsy, if excessive scar tissue growth or extracellular matrix deposition occurs around the VNS leads, this can reduce the efficacy of the device. VNS devices that release a therapeutic agent able to reducing scarring at the electrode-tissue interface can increase the efficiency of impulse transmission and increase the duration that these devices function clinically for the treatment of depression, anxiety, obesity, sleep disorders and dementia. In one aspect, the device includes VNS devices and/or leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof can be infiltrated into the tissue surrounding the vagus nerve where the lead will be implanted.

e) Sacral Nerve Stimulation for Bladder Control Problems

Sacral nerve stimulation is used in the management of patients with urinary control problems such as urge incontinence, nonobstructive urinary retention, or urgency-frequency. Millions of people suffer from bladder control problems and a significant percentage (estimated to be in excess of 60%) is not adequately treated by other available therapies such as medications, absorbent pads, external collection devices, bladder augmentation or surgical correction. This can be a debilitating medical problem that can cause severe social anxiety and cause people to become isolated and depressed.

Mild electrical stimulation of the sacral nerve is used to influence the functioning of the bladder, urinary sphincter, and the pelvic floor muscles (all structures which receive nerve supply from the sacral nerve). An electrical lead is surgically implanted adjacent to the sacral nerve and a neurostimulator is implanted subcutaneously in the upper buttock or abdomen; the two are connected by an extension. The use of tined leads allows sutureless anchoring of the leads and minimally-invasive placement of the leads under local anesthesia. A handheld programmer is available for adjustment of the device by the attending physician and a patient-controlled programmer is available to adjust the settings and to turn the device on and off. The pulses are adjusted to provide bladder control and relieve the patient's symptoms.

Several neurostimulation systems have been described for sacral nerve stimulation in which electrical stimulation is targeted towards the bladder, pelvic floor muscles, bowel and/or sexual organs. For example, the neurostimulator may be an electrical stimulation system composed of an electrical stimulator and leads having insulator sheaths, which may be anchored in the sacrum using minimally-invasive surgery. See e.g., U.S. Pat. No. 5,957,965. In another aspect, the neurostimulator may be used to condition pelvic, sphincter or bladder muscle tissue. For example, the neurostimulator may be intramuscular electrical stimulator composed of a pulse generator and an elongated medical lead that is used for electrically stimulating or sensing electrical signals originating from muscle tissue. See e.g., U.S. Pat. No. 6,434,431. Another neurostimulation system consists of a leadless, tubular-shaped microstimulator that is implanted at pelvic floor muscles or associated nerve tissue that need to be stimulated to treat urinary incontinence. See e.g., U.S. Pat. No. 6,061,596.

A commercially available example of a neurostimulation system to treat bladder conditions is the INTERSTIM Sacral Nerve Stimulation System made by Medtronic, Inc. See e.g., U.S. Pat. Nos. 6,104,960; 6,055,456 and 5,957,965.

Regardless of the specific design features, for bladder control therapy to be effective, the leads must be accurately positioned adjacent to the sacral nerve, bladder, sphincter or pelvic muscle (depending upon the particular system employed). If excessive scar tissue growth or extracellular matrix deposition occurs around the leads, efficacy can be compromised. Sacral nerve stimulating devices (such as INTERSTIM) that release a therapeutic agent able to reducing scarring at the electrode-tissue interface can increase the efficiency of impulse transmission and increase the duration that these devices function clinically. In one aspect, the device includes sacral nerve stimulating devices and/or leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be infiltrated into the tissue surrounding the sacral nerve where the lead will be implanted.

For devices designed to stimulate the bladder or pelvic muscle tissue directly, slightly different embodiments may be required. In this aspect, the device includes bladder or pelvic muscle stimulating devices, leads, and/or sensors that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be directly infiltrated into the muscle tissue itself (preferably adjacent to the lead and/or sensor that is delivering an impulse or monitoring the activity of the muscle).

f) Gastric Nerve Stimulation for the Treatment of GI Disorders

Neurostimulator of the gastric nerve (which supplies the stomach and other portions of the upper GI tract) is used to influence gastric emptying and satiety sensation in the management of clinically significant obesity or problems associated with impaired GI motility. Morbid obesity has reached epidemic proportions and is thought to affect over 25 million Americans and lead to significant health problems such as diabetes, heart attack, stroke and death. Mild electrical stimulation of the gastric nerve is used to influence the functioning of the upper GI tract and stomach (all structures which receive nerve supply from the gastric nerve). An electrical lead is surgically implanted adjacent to the gastric nerve and a neurostimulator is implanted subcutaneously; the two are connected by an extension. A handheld programmer is available for adjustment of the device by the attending physician and a patient-controlled programmer is available to adjust the settings and to turn the device on and off. The pulses are adjusted to provide a sensation of satiety and relieve the sensation of hunger experienced by the patient. This can reduce the amount of food (and hence caloric) intake and allow the patient to lose weight successfully. Related devices include neurostimulation devices used to stimulate gastric emptying in patients with impaired gastric motility, a neurostimulator to promote bowel evacuation in patients with constipation (stimulation is delivered to the colon), and devices targeted at the bowel for patients with other GI motility disorders.

Several such devices have been described including, for example, a sensor that senses electrical activity in the gastrointestinal tract which is coupled to a pulse generator that emits and inhibits asynchronous stimulation pulse trains based on the natural gastrointestinal electrical activity. See e.g., U.S. Pat. No. 5,995,872. Other neurostimulation devices deliver impulses to the colon and rectum to manage constipation and are composed of electrical leads, electrodes and an implanted stimulation generator. See e.g., U.S. Pat. No. 6,026,326. The neurostimulator may be a pulse generator and electrodes that electrically stimulate the neuromuscular tissue of the viscera to treat obesity. See e.g., U.S. Pat. No. 6,606,523. The neurostimulator may be a hermetically sealed implantable pulse generator that is electrically coupled to the gastrointestinal tract and emits two rates of electrical stimulation to treat gastroparesis for patients with impaired gastric emptying. See e.g., U.S. Pat. No. 6,091,992. The neurostimulator may be composed of an electrical signal controller, connector wire and attachment lead which generates continuous low voltage electrical stimulation to the fundus of the stomach to control appetite. See e.g. U.S. Pat. No. 6,564,101. Other neurostimulators that are used to electrically stimulate the gastrointestinal tract are described in, e.g., U.S. Pat. Nos. 6,453,199; 6,449,511 and 6,243,607.

Another example of a gastric nerve stimulation device for use with the present invention is the TRANSCEND Implantable Gastric Stimulator (IGS), which is currently being developed by Transneuronix, Inc. (Mt. Arlington, N.J.). The IGS is a programmable, bipolar pulse generator that delivers small bursts of electrical pulses through the lead to the stomach wall to treat obesity. See, e.g., U.S. Pat. Nos. 6,684,104 and 6,165,084.

Regardless of the specific design features, for gastric nerve stimulation to be effective in satiety control (or gastroparesis), the leads must be accurately positioned adjacent to the gastric nerve. If excessive scar tissue growth or extracellular matrix deposition occurs around the leads, efficacy can be compromised. Gastric nerve stimulating devices (and other implanted devices designed to influence GI motility) that release a therapeutic agent able to reduce scarring at the electrode-tissue interface can increase the efficiency of impulse transmission and increase the duration that these devices function clinically. In one aspect, the device includes gastric nerve stimulating devices and/or leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be infiltrated into the tissue surrounding the gastric nerve where the lead will be implanted.

g) Cochlear Implants for the Treatment of Deafness

Neurostimulation is also used in the form of a cochlear implant that stimulates the auditory nerve for correcting sensorineural deafness. A sound processor captures sound from the environment and processes it into a digital signal that is transmitted via an antenna through the skin to the cochlear implant. The cochlear implant, which is surgically implanted in the cochlea adjacent to the auditory nerve, converts the digital information into electrical signals that are communicated to the auditory nerve via an electrode array. Effectively, the cochlear implant serves to bypass the nonfunctional cochlear transducers and directly depolarize afferent auditory nerve fibers. This stimulates the nerve to send signals to the auditory center in the brain and allows the patient to “hear” the sounds detected by the sound processor. The treatment is used for adults with 70 dB or greater hearing loss (and able to understand up to 50% of words in a sentence using a hearing aid) or children 12 months or older with 90 dB hearing loss in both ears.

Although many implantations are performed without incident, approximately 12-15% of patients experience some complications. Histologic assessment of cochlear implants has revealed that several forms of injury and scarring can occur. Surgical trauma can induce cochlear fibrosis, cochlear neossification and injury to the membranous cochlea (including loss of the sensorineural elements). A foreign body reaction along the implant and the electrode can produce a fibrous tissue response along the electrode array that has been associated with implant failure. Coating the implant and/or the electrode with an anti-scarring composition may help reduce the incidence of failure. As an alternative, or in addition to this, fibrosis may be reduced or prevented by the infiltration of an anti-scarring drug combination, or individual component(s) thereof, into the tissue (the scala tympani) where the electrodes contact the auditory nerve fibers.

A variety of suitable cochlear implant systems or “bionic ears” have been described for use in association with this invention. For example, the neurostimulator may be composed of a plurality of transducer elements which detect vibrations and then generates a stimulus signal to a corresponding neuron connected to the cranial nerve. See e.g., U.S. Pat. No. 5,061,282. The neurostimulator may be a cochlear implant having a sound-to-electrical stimulation encoder, a body implantable receiver-stimulator and electrodes, which emit pulses based on received electrical signals. See e.g., U.S. Pat. No. 4,532,930. The neurostimulator may be an intra-cochlear apparatus that is composed of a transducer that converts an audio signal into an electrical signal and an electrode array which electrically stimulates predetermined locations of the auditory nerve. See e.g., U.S. Pat. No. 4,400,590. The neurostimulator may be a stimulus generator for applying electrical stimuli to any branch of the 8^{th} nerve in a generally constant rate independent of audio modulation, such that it is perceived as active silence. See e.g., U.S. Pat. No. 6,175,767. The neurostimulator may be a subcranially implanted electromechanical system that has an input transducer and an output stimulator that converts a mechanical sound vibration into an electrical signal. See e.g., U.S. Pat. No. 6,235,056. The neurostimulator may be a cochlear implant that has a rechargeable battery housed within the implant for storing and providing electrical power. See e.g., U.S. Pat. No. 6,067,474. Other neurostimulators that are used as cochlear implants are described in, e.g., U.S. Pat. Nos. 6,358,281; 6,308,101 and 5,603,726.

Several commercially available devices are available for the treatment of patients with significant sensorineural hearing loss and are suitable for use with the present invention. For example, the HIRESOLUTION Bionic Ear System (Boston Scientific Corp., Nattick, Mass.) consists of the HIRES AURIA Processor which processes sound and sends a digital signal to the HIRES 90K Implant that has been surgically implanted in the inner ear. See e.g., U.S. Pat. Nos. 6,636,768; 6,309,410 and 6,259,951. The electrode array that transmits the impulses generated by the HIRES 90K Implant to the nerve may benefit from an anti-scarring coating and/or the infiltration of an anti-scarring drug combination, or individual component(s) thereof, into the region around the electrode-nerve interface. The PULSARci cochlear implant (MED-EL GMBH, Innsbruck, Austria, see e.g., U.S. Pat. Nos. 6,556,870 and 6,231,604) and the NUCLEUS 3 cochlear implant system (Cochlear Corp., Lane Cove, Australia, see e.g., U.S. Pat. Nos. 6,807,445; 6,788,790; 6,554,762; 6,537,200 and 6,394,947) are other commercial examples of cochlear implants whose electrodes are suitable for coating with an anti-scarring composition (or infiltration of an anti-scarring drug combination, or individual component(s) thereof, into the region around the electrode-nerve interface) under the present invention.

Regardless of the specific design features, for cochlear implants to be effective in sensorineural deafness, the electrode arrays must be accurately positioned adjacent to the afferent auditory nerve fibers. If excessive scar tissue growth or extracellular matrix deposition occurs around the leads, efficacy can be compromised. Cochlear implants that release a therapeutic agent able to reduce scarring at the electrode-tissue interface can increase the efficiency of impulse transmission and increase the duration that these devices function clinically. In one aspect, the device includes cochlear implants and/or leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be infiltrated into the cochlear tissue surrounding the lead.

h) Electrical Stimulation to Promote Bone Growth

In another aspect, electrical stimulation can be used to stimulate bone growth. For example, the stimulation device may be an electrode and generator having a strain response piezoelectric material which responds to strain by generating a charge to enhance the anchoring of an implanted bone prosthesis to the natural bone. See e.g., U.S. Pat. No. 6,143,035. If excessive scar tissue growth or extracellular matrix deposition occurs around the leads, efficacy can be compromised. Electrical bone stimulation devices that release a therapeutic agent able to reduce scarring at the electrode-tissue interface can increase the efficiency of impulse transmission and increase the duration that these devices function clinically. In one aspect, the device includes bone stimulation devices and/or leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be infiltrated into the bone tissue surrounding the electrical lead.

Although numerous neurostimulation devices have been described above, all possess similar design features and cause similar unwanted tissue reactions following implantation. It should be obvious to one of skill in the art that commercial neurostimulation devices not specifically cited above as well as next-generation and/or subsequently-developed commercial neurostimulation products are to be anticipated and are suitable for use under the present invention. The neurostimulation device, particularly the lead(s), must be positioned in a very precise manner to ensure that stimulation is delivered to the correct anatomical location in the nervous system. All, or parts, of a neurostimulation device can migrate following surgery, or excessive scar (or glial) tissue growth can occur around the implant, which can lead to a reduction in the performance of these devices. Neurostimulator devices that release a therapeutic agent for reducing scarring (or gliosis) at the electrode-tissue interface can be used to increase the efficacy and/or the duration of activity of the implant (particularly for fully-implanted, battery-powered devices). In one aspect, the present invention provides neurostimulator devices that include an anti-scarring (or anti-gliosis) drug combination, or individual component(s) thereof, or a composition that includes an anti-scarring (or anti-gliosis) drug combination, or individual component(s) thereof. Numerous polymeric and non-polymeric delivery systems for use in neurostimulator devices will be described below. These compositions can further include one or more fibrosis-inhibiting (or gliosis-inhibiting) drug combination, or individual component(s) thereof, such that the overgrowth of granulation, fibrous, or gliotic tissue is inhibited or reduced.

Methods for incorporating fibrosis-inhibiting (or gliosis-inhibiting) compositions onto or into these neurostimulator devices include: (a) directly affixing to the device, lead and/or the electrode a fibrosis-inhibiting (or gliosis-inhibiting) composition (e.g., by either a spraying process or dipping process as described above, with or without a carrier); (b) directly incorporating into the device, lead and/or the electrode a fibrosis-inhibiting (or gliosis-inhibiting) composition (e.g., by either a spraying process or dipping process as described above, with or without a carrier); (c) by coating the device, lead and/or the electrode with a substance such as a hydrogel which may in turn absorb the fibrosis-inhibiting (or gliosis-inhibiting) composition; (d) by interweaving fibrosis-inhibiting (or gliosis-inhibiting) composition coated thread (or the polymer itself formed into a thread) into the device, lead and/or electrode structure; (e) by inserting the device, lead and/or the electrode into a sleeve or mesh which is comprised of, or coated with, a fibrosis-inhibiting (or gliosis-inhibiting) composition; (f) constructing the device, lead and/or the electrode itself (or a portion of the device and/or the electrode) with a fibrosis-inhibiting (or gliosis-inhibiting) composition; or (g) by covalently binding the fibrosis-inhibiting (or gliosis-inhibiting) agent directly to the device, lead and/or electrode surface or to a linker (small molecule or polymer) that is coated or attached to the device surface. Each of these methods illustrates an approach for combining an electrical device with a fibrosis-inhibiting (also referred to herein as an anti-scarring) or gliosis-inhibiting drug combination, or individual component(s) thereof, according to the present invention.

For these devices, leads and electrodes, the coating process can be performed in such a manner as to: (a) coat the non-electrode portions of the lead or device; (b) coat the electrode portion of the lead; or (c) coat all or parts of the entire device with the fibrosis-inhibiting (or gliosis-inhibiting) composition. Additionally, or alternatively, the fibrosis-inhibiting (or gliosis-inhibiting) drug combination, or individual component(s) thereof, can be mixed with the materials that are used to make the device, lead and/or electrode such that the fibrosis-inhibiting (or gliosis-inhibiting) drug combination, or individual component(s) thereof, is incorporated into the final product. In these manners, a medical device may be prepared which has a coating, where the coating is, e.g., uniform, non-uniform, continuous, discontinuous, or patterned.

In another aspect, a neurostimulation device may include a plurality of reservoirs within its structure, each reservoir configured to house and protect a therapeutic drug combination, or individual component(s) thereof. The reservoirs may be formed from divets in the device surface or micropores or channels in the device body. In one aspect, the reservoirs are formed from voids in the structure of the device. The reservoirs may house a single drug combination, or individual component(s) thereof, or more than one drug combination, or individual component(s) thereof. The drug combination(s), or individual component(s) thereof, may be formulated with a carrier (e.g., a polymeric or non-polymeric material) that is loaded into the reservoirs. The filled reservoir can function as a drug delivery depot which can release drug combination, or individual component(s) thereof, over a period of time dependent on the release kinetics of the drug(s) from the carrier. In certain embodiments, the reservoir may be loaded with a plurality of layers. Each layer may include a different drug combination, or individual component(s) thereof, having a particular amount (dose) of drug combination, or individual component(s) thereof, and each layer may have a different composition to further tailor the amount of drug that is released from the substrate. The multi-layered carrier may further include a barrier layer that prevents release of the drug(s). The barrier layer can be used, for example, to control the direction that the drug elutes from the void. Thus, the coating of the medical device may directly contact the electrical device, or it may indirectly contact the electrical device when there is something, e.g., a polymer layer, that is interposed between the electrical device and the coating that contains the fibrosis-inhibiting (or gliosis-inhibiting) drug combination, or individual component(s) thereof.

In addition to, or as an alternative to incorporating a fibrosis-inhibiting (or gliosis-inhibiting) drug combination, or individual component(s) thereof, onto or into the neurostimulation device, the fibrosis-inhibiting (or gliosis-inhibiting) drug combination, or individual component(s) thereof, can be applied directly or indirectly to the tissue adjacent to the neurostimulator device (preferably near the electrode-tissue interface). This can be accomplished by applying the fibrosis-inhibiting (or gliosis inhibiting) drug combination, or individual component(s) thereof, with or without a polymeric, non-polymeric, or secondary carrier: (a) to the lead and/or electrode surface (e.g., as an injectable, paste, gel or mesh) during the implantation procedure); (b) to the surface of the tissue (e.g., as an injectable, paste, gel, in situ forming gel or mesh) prior to, immediately prior to, or during, implantation of the neurostimulation device, lead and/or electrode; (c) to the surface of the lead and/or electrode and/or the tissue surrounding the implanted lead and/or electrode (e.g., as an injectable, paste, gel, in situ forming gel or mesh) immediately after to the implantation of the neurostimulation device, lead and/or electrode; (d) by topical application of the anti-fibrosis (or anti-gliosis) drug combination, or individual component(s) thereof, into the anatomical space where the neurostimulation device, lead and/or electrode will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the device will be inserted); (e) via percutaneous injection into the tissue surrounding the device, lead and/or electrode as a solution as an infusate or as a sustained release preparation; or (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

It should be noted that certain polymeric carriers themselves can help prevent the formation of fibrous or gliotic tissue around the neuroimplant. These carriers (to be described shortly) are particularly useful for the practice of this embodiment, either alone, or in combination with a fibrosis-inhibiting (or gliosis-inhibiting) composition. The following polymeric carriers can be infiltrated (as described in the previous paragraph) into the vicinity of the electrode-tissue interface and include: (a) sprayable collagen-containing formulations such as COSTASIS and crosslinked derivatized poly(ethylene glycol)-collagen compositions (described, e.g., in U.S. Pat. Nos. 5,874,500 and 5,565,519 and referred to herein as “CT3” (both from Angiotech Pharmaceuticals, Inc., Canada), either alone, or loaded with a fibrosis-inhibiting (or gliosis-inhibiting) drug combination, or individual component(s) thereof, applied to the implantation site (or the implant/device surface); (b) sprayable PEG-containing formulations such as COSEAL (Angiotech Pharmaceuticals, Inc.), FOCALSEAL (Genzyme Corporation, Cambridge, Mass.), SPRAYGEL or DURASEAL (both from Confluent Surgical, Inc., Boston, Mass.), either alone, or loaded with a fibrosis-inhibiting (or gliosis-inhibiting) drug combination, or individual component(s) thereof, applied to the implantation site (or the implant/device surface); (c) fibrinogen-containing formulations such as FLOSEAL or TISSEAL (both from Baxter Healthcare Corporation, Fremont, Calif.), either alone, or loaded with a fibrosis-inhibiting (or gliosis-inhibiting) drug combination, or individual component(s) thereof, applied to the implantation site (or the implant/device surface); (d) hyaluronic acid-containing formulations such as RESTYLANE or PERLANE (both from Q-Med AB, Sweden), HYLAFORM (Inamed Corporation, Santa Barbara, Calif.), SYNVISC (Biomatrix, Inc., Ridgefield, N.J.), SEPRAFILM or SEPRACOAT (both from Genzyme Corporation), loaded with a fibrosis-inhibiting (or gliosis-inhibiting) drug combination, or individual component(s) thereof, applied to the implantation site (or the implant/device surface); (e) polymeric gels for surgical implantation such as REPEL (Life Medical Sciences, Inc., Princeton, N.J.) or FLOWGEL (Baxter Healthcare Corporation) loaded with a fibrosis-inhibiting (or gliosis-inhibiting) drug combination, or individual component(s) thereof, applied to the implantation site (or the implant/device surface); (f) orthopedic “cements” used to hold prostheses and tissues in place loaded with a fibrosis-inhibiting (or gliosis-inhibiting) drug combination, or individual component(s) thereof, applied to the implantation site (or the implant/device surface), such as OSTEOBOND (Zimmer, Inc., Warsaw, Ind.), low viscosity cement (LVC); Wright Medical Technology, Inc., Arlington, Tenn.), SIMPLEX P (Stryker Corporation, Kalamazoo, Mich.), PALACOS (Smith & Nephew Corporation, United Kingdom), and ENDURANCE (Johnson & Johnson, Inc., New Brunswick, N.J.); (g) surgical adhesives containing cyanoacrylates such as DERMABOND (Johnson & Johnson, Inc.), INDERMIL (U.S. Surgical Company, Norwalk, Conn.), GLUSTITCH (Blacklock Medical Products Inc., Canada), TISSUEMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.), either alone, or loaded with a fibrosis-inhibiting (or gliosis-inhibiting) drug combination, or individual component(s) thereof, applied to the implantation site (or the implant/device surface); (h) implants containing hydroxyapatite [or synthetic bone material such as calcium sulfate, VITOSS and CORTOSS (both from Orthovita, Inc., Malvern, Pa.) loaded with a fibrosis-inhibiting (or gliosis-inhibiting) drug combination, or individual component(s) thereof, applied to the implantation site (or the implant/device surface); (i) other biocompatible tissue fillers loaded with a fibrosis-inhibiting (or gliosis-inhibiting) drug combination, or individual component(s) thereof, such as those made by BioCure, Inc. (Norcross, Ga.), 3M Company (St. Paul, Minn.) and Neomend, Inc. (Sunnyvale, Calif.), applied to the implantation site (or the implant/device surface); (j) polysaccharide gels such as the ADCON series of gels (available from Gliatech, Inc., Cleveland, Ohio) either alone, or loaded with a fibrosis-inhibiting (or gliosis-inhibiting) drug combination, or individual component(s) thereof, applied to the implantation site (or the implant/device surface); and/or (k) films, sponges or meshes such as INTERCEED (Gynecare Worldwide, a division of Ethicon, Inc., Somerville, N.J.), VICRYL mesh (Ethicon, Inc.), and GELFOAM (Pfizer, Inc., New York, N.Y.) loaded with a fibrosis-inhibiting (or gliosis-inhibiting) drug combination, or individual component(s) thereof, applied to the implantation site (or the implant/device surface).

A preferred polymeric matrix which can be used to help prevent the formation of fibrous or gliotic tissue around the neuroimplant, either alone or in combination with a fibrosis-inhibiting (or gliosis-inhibiting) drug combination, or individual component(s) thereof, or a composition comprising a drug combination, or individual component(s) thereof, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic drug combination, or individual component(s) thereof, or a stand-alone composition to help prevent the formation of fibrous or gliotic tissue around the neuroimplant.

It should be apparent to one of skill in the art that potentially any anti-scarring (or anti-gliotic) drug combination, or individual component(s) thereof, described above may be utilized alone, or in combination, in the practice of this embodiment. As neurostimulator devices are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug(s) to the device (i.e., as a coating or infiltrated into the surrounding tissue), the anti-scarring drug combination(s), or individual component(s) thereof, used alone or in combination, may be administered under the following dosing guidelines:

Drugs and dosage: Exemplary anti-scarring drug combinations that may be used include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for neurostimulation devices will depend on a variety of factors, including the type of formulation, the location of the treatment site, and the type of condition being treated, as well as the surface area of the device. However, certain principles can be applied in the application of this art. Drug dose can be calculated as a function of dose per unit area (of the treatment site), wherein the total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring or anti-gliosis drug combination, or individual component(s) thereof, is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In certain embodiments, the drug is released in effective concentrations for a period ranging from 1-90 days. It should be understood in certain embodiments that within the drug combination, one drug may be released at a different rate and/or for a different amount of time than the other drug(s).

The exemplary anti-fibrosing or anti-gliosis drug combinations or individual components thereof should be administered under the following dosing guidelines. The total amount (dose) of anti-scarring or anti-gliosis agent(s) in the drug combinations or compositions that comprise the drug combinations can be in the range of about 0.01 μg-10 μg, or 10 μg-100 μg, or 100 μg-1000 μg, or 1 mg-10 mg, or 10 mg-250 mg, or 250 mg-1000 mg, or 1000 mg-2500 mg. The dose (amount) of anti-scarring or anti-gliosis agent(s) per unit area of surface to which the agent is applied may be in the range of about 0.01 μg/mm²-1 μg/mm², or 1 μg/mm²-10 μg/mm², or 10 μg/mm²-250 μg/mm², or 250 μg/mm²-1000 μg/mm², or 1000 μg/mm²-2500 μg/mm².

Provided below are exemplary drug combinations and dosage ranges for various anti-scarring and/or anti-gliosis drug combinations or individual components thereof that can be used in conjunction with neurostimulation devices in accordance with the invention.

Exemplary anti-fibrotic drug combinations for description of dosing include, but are not limited to amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate and analogues and derivatives thereof: total dose of each drug within the combination not to exceed 500 mg (range of 0.1 μg to 500 mg; preferred 1 μg to 200 mg). Dose per unit area of 0.01 μg/mm² to 200 μg/mm²; preferred dose of 0.1 μg/mm² to 100 μg/mm². Minimum concentration of 10⁻⁸ to 10⁻⁴M of agent is to be maintained on the implant or barrier surface. Molar ratio of each drug in the combination is to be within the range of 1:1 to 1:1000. Molar ratios within this range may include but are not limited to 1:5, 1:10, 1:15, 1:20, 1:30, 1:50, 1:75, 1:100, 1:200, 1:500, 1:1000. Note that molar ratios may also lie between the ratios stated above.

2) Cardiac Rhythm Management (CRM) Devices

In another aspect, the electrical device may be a cardiac pacemaker device where a pulse generator delivers an electrical impulse to myocardial tissue (often specialized conduction fibres) via an implanted lead in order to regulate cardiac rhythm. Typically, electrical leads are composed of a connector assembly, a lead body (i.e., conductor) and an electrode. Electrical leads may be unipolar, in which they are adapted to provide effective therapy with only one electrode. Multi-polar leads are also available, including bipolar, tripolar and quadripolar leads. Electrical leads may also have insulating sheaths which may include polyurethane or silicone-rubber coatings. Representative examples of electrical leads include, without limitation, medical leads, cardiac leads, pacer leads, pacing leads, pacemaker leads, endocardial leads, endocardial pacing leads, cardioversion/defibrillator leads, cardioversion leads, epicardial leads, epicardial defibrillator leads, patch defibrillators, patch leads, electrical patch, transvenous leads, active fixation leads, passive fixation leads and sensing leads Representative examples of CRM devices that utilize electrical leads include: pacemakers, LVAD's, defibrillators, implantable sensors and other electrical cardiac stimulation devices.

There are numerous pacemaker devices where the occurrence of a fibrotic reaction will adversely affect the functioning of the device or cause damage to the myocardial tissue. Typically, fibrotic encapsulation of the pacemaker lead (or the growth of fibrous tissue between the lead and the target myocardial tissue) slows, impairs, or interrupts electrical transmission of the impulse from the device to the myocardium. For example, fibrosis is often found at the electrode-myocardial interfaces in the heart, which may be attributed to electrical injury from focal points on the electrical lead. The fibrotic injury may extend into the tricuspid valve, which may lead to perforation. Fibrosis may lead to thrombosis of the subclavian vein; a condition which may be life threatening. Electrical leads that release therapeutic agent for reducing scarring at the electrode-tissue interface may help prolong the clinical performance of these devices. Not only can fibrosis cause the device to function suboptimally or not at all, it can cause excessive drain on battery life as increased energy is required to overcome the electrical resistance imposed by the intervening scar tissue. Similarly, fibrotic encapsulation of the sensing components of a rate-responsive pacemaker (described below) can impair the ability of the pacemaker to identify and correct rhythm abnormalities leading to inappropriate pacing of the heart or the failure to function correctly when required.

Several different electrical pacing devices are used in the treatment of various cardiac rhythm abnormalities including pacemakers, implantable cardioverter defibrillators (ICD), left ventricular assist devices (LVAD), and vagus nerve stimulators (stimulates the fibers of the vagus nerve which in turn innervate the heart). The pulse generating portion of device sends electrical impulses via implanted leads to the muscle (myocardium) or conduction tissue of the heart to affect cardiac rhythm or contraction. Pacing can be directed to one or more chambers of the heart. Cardiac pacemakers may be used to block, mask, or stimulate electrical signals in the heart to treat dysfunctions, including, without limitation, atrial rhythm abnormalities, conduction abnormalities and ventricular rhythm abnormalities. ICDs are used to depolarize the ventricals and re-establish rhythm if a ventricular arrhythmia occurs (such as asystole or ventricular tachycardia) and LVADs are used to assist ventricular contraction in a failing heart.

Representative examples of patents which describe pacemakers and pacemaker leads include U.S. Pat. Nos. 4,662,382, 4,782,836, 4,856,521, 4,860,751, 5,101,824, 5,261,419, 5,284,491, 6,055,454, 6,370,434, and 6,370,434. Representative examples of electrical leads include those found on a variety of cardiac devices, such as cardiac stimulators (see e.g., U.S. Pat. Nos. 6,584,351 and 6,115,633), pacemakers (see e.g., U.S. Pat. Nos. 6,564,099; 6,246,909 and 5,876,423), implantable cardioverter-defibrillators (ICDs), other defibrillator devices (see e.g., U.S. Pat. No. 6,327,499), defibrillator or demand pacer catheters (see e.g., U.S. Pat. No. 5,476,502) and Left Ventricular Assist Devices (see e.g., U.S. Pat. No. 5,503,615).

Cardiac rhythm devices, and in particular the lead(s) that deliver the electrical pulsation, must be positioned in a very precise manner to ensure that stimulation is delivered to the correct anatomical location in the heart. All, or parts, of a pacing device can migrate following surgery, or excessive scar tissue growth can occur around the lead, which can lead to a reduction in the performance of these devices (as described previously). Cardiac rhythm management devices that release a therapeutic drug combination, or individual component(s) thereof, for reducing scarring at the electrode-tissue interface can be used to increase the efficacy and/or the duration of activity (particularly for fully-implanted, battery-powered devices) of the implant. Accordingly, the present invention provides cardiac leads that are coated with an anti-scarring drug combination, or individual component(s) thereof, or a composition that includes an anti-scarring drug combination, or individual component(s) thereof.

For greater clarity, several specific cardiac rhythm management devices and treatments will be described in greater detail including:

a) Cardiac Pacemakers

Cardiac rhythm abnormalities are extremely common in clinical practice and the incidence increases in frequency with both age and the presence of underlying coronary artery disease or myocardial infarction. A litany of arrythmias exists, but they are generally categorized into conditions where the heart beats too slowly (bradyarrythmias—such heart block, sinus node dysfunction) or too quickly (tachyarrhythmias—such as atrial fibrillation, WPW syndrome, ventricular fibrillation). A pacemaker functions by sending an electrical pulse (a pacing pulse) that travels via an electrical lead to the electrode (at the tip of the lead) which delivers an electrical impulse to the heart that initiates a heartbeat. The leads and electrodes can be located in one chamber (either the right atrium or the right ventricle—called single-chamber pacemakers) or there can be electrodes in both the right atrium and the right ventricle (called dual-chamber pacemakers). Electrical leads may be implanted on the exterior of the heart (e.g., epicardial leads) by a surgical procedure, or they can be connected to the endocardial surface of the heart via a catheter, guidewire or stylet. In some pacemakers, the device assumes the rhythm generating function of the heart and fires at a regular rate. In other pacemakers, the device merely augments the heart's own pacing function and acts “on demand” to provide pacing assistance as required (called “adaptive-rate” pacemakers); the pacemaker receives feedback on heart rhythm (and hence when to fire) from an electrode sensor located on the lead. Other pacemakers, called rate responsive pacemakers, have special sensors that detect changes in body activity (such as movement of the arms and legs, respiratory rate) and adjust pacing up or down accordingly.

Numerous pacemakers and pacemaker leads are suitable for use in this invention. For example, the pacing lead may have an increased resistance to fracture by being composed of an elongated coiled conductor mounted within a lumen of a lead body whereby it may be coupled electrically to a stranded conductor. See e.g., U.S. Pat. Nos. 6,061,598 and 6,018,683. The pacing lead may have a coiled conductor with an insulated sheath, which has a resistance to crush fatigue in the region between the rib and clavicle. See e.g., U.S. Pat. No. 5,800,496. The pacing lead may be expandable from a first, shorter configuration to a second, longer configuration by being composed of slideable inner and outer overlapping tubes containing a conductor. See e.g., U.S. Pat. No. 5,897,585. The pacing lead may have the means for temporarily making the first portion of the lead body stiffer by using a magnet-rheologic fluid in a cavity that stiffens when exposed to a magnetic field. See e.g., U.S. Pat. No. 5,800,497. The pacing lead may be a coil configuration composed of a plurality of wires or wire bundles made from a duplex titanium alloy. See e.g., U.S. Pat. No. 5,423,881. The pacing lead may be composed of a wire wound in a coil configuration with the wire composed of stainless steel having a composition of at least 22% nickel and 2% molybdenum. See e.g., U.S. Pat. No. 5,433,744. Other pacing leads are described in, e.g., U.S. Pat. Nos. 6,489,562; 6,289,251 and 5,957,967.

In another aspect, the electrical lead used in the practice of this invention may have an active fixation element for attachment to tissue. For example, the electrical lead may have a rigid fixation helix with microgrooves that are dimensioned to minimize the foreign body response following implantation. See e.g., U.S. Pat. No. 6,078,840. The electrical lead may have an electrode/anchoring portion with a dual tapered self-propelling spiral electrode for attachment to vessel wall. See e.g., U.S. Pat. No. 5,871,531. The electrical lead may have a rigid insulative electrode head carrying a helical electrode. See e.g., U.S. Pat. No. 6,038,463. The electrical lead may have an improved anchoring sleeve designed with an introducer sheath to minimize the flow of blood through the sheath during introduction. See e.g., U.S. Pat. No. 5,827,296. The electrical lead may be composed of an insulated electrical conductive portion and a lead-in securing section having a longitudinally rigid helical member which may be screwed into tissue. See e.g., U.S. Pat. No. 4,000,745.

Suitable leads for use in the practice of this invention also include multi-polar leads with multiple electrodes connected to the lead body. For example, the electrical lead may be a multi-electrode lead whereby the lead has two internal conductors and three electrodes with two electrodes coupled by a capacitor integral with the lead. See e.g., U.S. Pat. No. 5,824,029. The electrical lead may be a lead body with two straight sections and a bent third section with associated conductors and electrodes whereby the electrodes are bipolar. See e.g., U.S. Pat. No. 5,995,876. In another aspect, the electrical lead may be implanted by using a catheter, guidewire or stylet. For example, the electrical lead may be composed of an elongated insulative lead body having a lumen with a conductor mounted within the lead body and a resilient seal having an expandable portion through which a guidewire may pass. See e.g., U.S. Pat. No. 6,192,280.

Commercially available pacemakers suitable for the practice of the invention include the KAPPA SR 400 Series single-chamber rate-responsive pacemaker system, the KAPPA DR 400 Series dual-chamber rate-responsive pacemaker system, the KAPPA 900 and 700 Series single-chamber rate-responsive pacemaker system, and the KAPPA 900 and 700 Series dual-chamber rate-responsive pacemaker system by Medtronic, Inc. Medtronic pacemaker systems utilize a variety leads including the CAPSURE Z Novus, CAPSUREFIX Novus, CAPSUREFIX, CAPSURE SP Novus, CAPSURE SP, CAPSURE EPI and the CAPSURE VDD which may be suitable for coating with a fibrosis-inhibiting drug combination, or individual component(s) thereof. Pacemaker systems and associated leads that are made by Medtronic are described in, e.g., U.S. Pat. Nos. 6,741,893; 5,480,441; 5,411,545; 5,324,310; 5,265,602; 5,265,601; 5,241,957 and 5,222,506. Medtronic also makes a variety of steroid-eluting leads including those described in, e.g., U.S. Pat. Nos. 5,987,746; 6,363,287; 5,800,470; 5,489,294; 5,282,844 and 5,092,332. The INSIGNIA single-chamber and dual-chamber system, PULSAR MAX II DR dual-chamber adaptive-rate pacemaker, PULSAR MAX II SR single-chamber adaptive-rate pacemaker, DISCOVERY II DR dual-chamber adaptive-rate pacemaker, DISCOVERY II SR single-chamber adaptive-rate pacemaker, DISCOVERY II DDD dual-chamber pacemaker, and the DISCOVERY II SSI dingle-chamber pacemaker systems made by Guidant Corp. (Indianapolis, Ind.) are also suitable pacemaker systems for the practice of this invention. Once again, the leads from the Guidant pacemaker systems may be suitable for coating with a fibrosis-inhibiting agent. Pacemaker systems and associated leads that are made by Guidant are described in, e.g., U.S. Pat. Nos. 6,473,648; 6,345,204; 6,321,122; 6,152,954; 5,769,881; 5,284,136; 5,086,773 and 5,036,849. The AFFINITY DR, AFFINITY VDR, AFFINITY SR, AFFINITY DC, ENTITY, IDENTITY, IDENTITY ADX, INTEGRITY, INTEGRITY μDR, INTEGRITY ADx, MICRONY, REGENCY, TRILOGY, and VERITY ADx, pacemaker systems and leads from St. Jude Medical, Inc. (St. Paul, Minn.) may also be suitable for use with a fibrosis-inhibiting coating to improve electrical transmission and sensing by the pacemaker leads. Pacemaker systems and associated leads that are made by St. Jude Medical are described in, e.g., U.S. Pat. Nos. 6,763,266; 6,760,619; 6,535,762; 6,246,909; 6,198,973; 6,183,305; 5,800,468 and 5,716,390. Alternatively, the fibrosis-inhibiting drug combination, or individual component(s) thereof, may be infiltrated into the region around the electrode-cardiac muscle interface under the present invention. It should be obvious to one of skill in the art that commercial pacemakers not specifically cited as well as next-generation and/or subsequently developed commercial pacemaker products are to be anticipated and are suitable for use under the present invention.

Regardless of the specific design features, for pacemakers to be effective in the management of cardiac rhythm disorders, the leads must be accurately positioned adjacent to the targeted cardiac muscle tissue. If excessive scar tissue growth or extracellular matrix deposition occurs around the leads, efficacy can be compromised. Pacemaker leads that release a therapeutic drug combination, or individual component(s) thereof, able to reduce scarring at the electrode-tissue and/or sensor-tissue interface, can increase the efficiency of impulse transmission and rhythm sensing, thereby increasing efficacy and battery longevity. In one aspect, the device includes pacemaker leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be infiltrated into the myocardial tissue surrounding the lead.

b) Implantable Cardioverter Defibrillator (ICD) Systems

Implantable cardioverter defibrillator (ICD) systems are similar to pacemakers (and many include a pacemaker system), but are used for the treatment of tachyarrhythmias such as ventricular tachycardia or ventricular fibrillation. An ICD consists of a mini-computer powered by a battery which is connected to a capacitor to helps the ICD charge and store enough energy to deliver therapy when needed. The ICD uses sensors to monitor the activity of the heart and the computer analysizes the data to determine when and if an arrhythmia is present. An ICD lead, which is inserted via a vein (called “transvenous” leads; in some systems the lead is implanted surgically—called an epicardial lead—and sewn onto the surface of the heart), connects into the pacing/computer unit. The lead, which is usually placed in the right ventricle, consists of an insulated wire and an electrode tip that contains a sensing component (to detect cardiac rhythm) and a shocking coil. A single-chamber ICD has one lead placed in the ventricle which defibrillates and paces the ventricle, while a dual-chamber ICD defibrillates the ventricle and paces the atrium and the ventricle. In some cases, an additional lead is required and is placed under the skin next to the rib cage or on the surface of the heart. In patients who require tachyarrhythmia management of the ventricle and atrium, a second coil is placed in the atrium to treat atrial tachycardia, atrial fibrillation and other arrhythmias. If a tachyarrhythmia is detected, a pulse is generated and propagated via the lead to the shocking coil which delivers a charge sufficient to depolarize the muscle and cardiovert or defibrillate the heart.

Several ICD systems have been described and are suitable for use in the practice of this invention. Representative examples of ICD's and associated components are described in U.S. Pat. Nos. 3,614,954, 3,614,955, 4,375,817, 5,314,430, 5,405,363, 5,607,385, 5,697,953, 5,776,165, 6,067,471, 6,169,923, and 6,152,955. Several ICD leads are suitable for use in the practice of this invention. For example, the defibrillator lead may be a linear assembly of sensors and coils formed into a loop which includes a conductor system for coupling the loop system to a pulse generator. See e.g., U.S. Pat. No. 5,897,586. The defibrillator lead may have an elongated lead body with an elongated electrode extending from the lead body, such that insulative tubular sheaths are slideably mounted around the electrode. See e.g., U.S. Pat. No. 5,919,222. The defibrillator lead may be a temporary lead with a mounting pad and a temporarily attached conductor with an insulative sleeve whereby a plurality of wire electrodes are mounted. See e.g., U.S. Pat. No. 5,849,033. Other defibrillator leads are described in, e.g., U.S. Pat. No. 6,052,625. In another aspect, the electrical lead may be adapted to be used for pacing, defibrillating or both applications. For example, the electrical lead may be an electrically insulated, elongated, lead body sheath enclosing a plurality of lead conductors that are separated from contacting one another. See e.g., U.S. Pat. No. 6,434,430. The electrical lead may be composed of an inner lumen adapted to receive a stiffening member (e.g., guide wire) that delivers fluoro-visible media. See e.g., U.S. Pat. No. 6,567,704. The electrical lead may be a catheter composed of an elongated, flexible, electrically nonconductive probe contained within an electrically conductive pathway that transmits electrical signals, including a defibrillation pulse and a pacer pulse, depending on the need that is sensed by a governing element. See e.g., U.S. Pat. No. 5,476,502. The electrical lead may have a low electrical resistance and good mechanical resistance to cyclical stresses by being composed of a conductive wire core formed into a helical coil covered by a layer of electrically conductive material and an electrically insulating sheath covering. See e.g., U.S. Pat. No. 5,330,521. Other electrical leads that may be adapted for use in pacing and/or defibrillating applications are described in, e.g., U.S. Pat. No. 6,556,873.

Commercially available ICDs suitable for the practice of the invention include the GEM III DR dual-chamber ICD, GEM III VR ICD, GEM II ICD, GEM ICD, GEM III AT atrial and ventricular arrhythmia ICD, JEWEL AF dual-chamber ICD, MICRO JEWEL ICD, MICRO JEWEL II ICD, JEWEL Plus ICD, JEWEL ICD, JEWEL ACTIVE CAN ICD, JEWEL PLUS ACTIVE CAN ICD, MAXIMO DR ICD, MAXIMO VR ICD, MARQUIS DR ICD, MARQUIS VR system, and the INTRINSIC dual-chamber ICD by Medtronic, Inc. Medtronic ICD systems utilize a variety leads including the SPRINT FIDELIS, SPRINT QUATRO SECURE steroid-eluting bipolar lead, Subcutaneous Lead System Model 6996SQ subcutaneous lead, TRANSVENE 6937A transvenous lead, and the 6492 Unipolar Atrial Pacing Lead which may be suitable for coating with a fibrosis-inhibiting drug combination, or individual component(s) thereof. ICD systems and associated leads that are made by Medtronic are described in, e.g., U.S. Pat. Nos. 6,038,472; 5,849,031; 5,439,484; 5,314,430; 5,165,403; 5,099,838 and 4,708,145. The VITALITY 2 DR dual-chamber ICD, VITALITY 2 VR single-chamber ICD, VITALITY AVT dual-chamber ICD, VITALITY DS dual-chamber ICD, VITALITY DS VR single-chamber ICD, VITALITY EL dual-chamber ICD, VENTAK PRIZM 2 DR dual-chamber ICD, and VENTAK PRIZM 2 VR single-chamber ICD systems made by Guidant Corp. are also suitable ICD systems for the practice of this invention. Once again, the leads from the Guidant ICD systems may be suitable for coating with a fibrosis-inhibiting agent. Guidant sells the FLEXTEND Bipolar Leads, EASYTRAK Lead System, FINELINE Leads, and ENDOTAK RELIANCE ICD Leads. ICD systems and associated leads that are made by Guidant are described in, e.g., U.S. Pat. Nos. 6,574,505; 6,018,681; 5,697,954; 5,620,451; 5,433,729; 5,350,404; 5,342,407; 5,304,139 and 5,282,837. Biotronik, Inc. (Germany) sells the POLYROX Endocardial Leads, KENTROX SL Quadripolar ICD Leads, AROX Bipolar Leads, and MAPOX Bipolar Epicardial Leads (see e.g., U.S. Pat. Nos. 6,449,506; 6,421,567; 6,418,348; 6,236,893 and 5,632,770). The CONTOUR MD ICD, PHOTON μ DR ICD, PHOTON μ VR ICD, ATLAS+ HF ICD, EPIC HF ICD, EPIC+ HF ICD systems and leads from St. Jude Medical may also be suitable for use with a fibrosis-inhibiting coating to improve electrical transmission and sensing by the ICD leads (see e.g., U.S. Pat. Nos. 5,944,746; 5,722,994; 5,662,697; 5,542,173; 5,456,706 and 5,330,523). Alternatively, the fibrosis-inhibiting drug combination, or individual component(s) thereof, may be infiltrated into the region around the electrode-cardiac muscle interface under the present invention. It should be obvious to one of skill in the art that commercial ICDs not specifically cited as well as next-generation and/or subsequently developed commercial ICD products are to be anticipated and are suitable for use under the present invention.

Regardless of the specific design features, for ICDs to be effective in the management of cardiac rhythm disorders, the leads must be accurately positioned adjacent to the targeted cardiac muscle tissue. If excessive scar tissue growth or extracellular matrix deposition occurs around the leads, efficacy can be compromised. ICD leads that release a therapeutic drug combination, or individual component(s) thereof, able to reduce scarring at the electrode-tissue and/or sensor-tissue interface, can increase the efficiency of impulse transmission and rhythm sensing, thereby increasing efficacy, preventing inappropriate cardioversion, and improving battery longevity. In one aspect, the device includes ICD leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be infiltrated into the myocardial tissue surrounding the lead.

c) Vagus Nerve Stimulation for the Treatment of Arrhythmia

In another aspect, a neurostimulation device may be used to stimulate the vagus nerve and affect the rhythm of the heart. Since the vagus nerve provides innervation to the heart, including the conduction system (including the SA node), stimulation of the vagus nerve may be used to treat conditions such as supraventricular arrhythmias, angina pectoris, atrial tachycardia, atrial flutter, atrial fibrillation and other arrhythmias that result in low cardiac output.

As described above, in VNS a bipolar electrical lead is surgically implanted such that it transmits electrical stimulation from the pulse generator to the left vagus nerve in the neck. The pulse generator is an implanted, lithium carbon monofluoride battery-powered device that delivers a precise pattern of stimulation to the vagus nerve. The pulse generator can be programmed (using a programming wand) by the cardiologist to treat a specific arrhythmia.

Products such as these have been described, for example, in U.S. Pat. Nos. 6,597,953 and 6,615,085. For example, the neurostimulator may be a vagal-stimulation apparatus which generates pulses at a frequency that varies automatically based on the excitation rates of the vagus nerve. See e.g., U.S. Pat. Nos. 5,916,239 and 5,690,681. The neurostimulator may be an apparatus that detects characteristics of tachycardia based on an electrogram and delivers a preset electrical stimulation to the nervous system to depress the heart rate. See e.g., U.S. Pat. No. 5,330,507. The neurostimulator may be an implantable heart stimulation system composed of two sensors, one for atrial signals and one for ventricular signals, and a pulse generator and control unit, to ensure sympatho-vagal stimulation balance. See e.g., U.S. Pat. No. 6,477,418. The neurostimulator may be a device that applies electrical pulses to the vagus nerve at a programmable frequency that is adjusted to maintain a lower heart rate. See e.g., U.S. Pat. No. 6,473,644. The neurostimulator may provide electrical stimulation to the vagus nerve to induce changes to electroencephalogram readings as a treatment for epilepsy, while controlling the operation of the heart within normal parameters. See e.g., U.S. Pat. No. 6,587,727.

A commercial example of a VNS system is the product produced by Cyberonics Inc. that consists of the Model 300 and Model 302 leads, the Model 101 and Model 102R pulse generators, the Model 201 programming wand and Model 250 programming software, and the Model 220 magnets. These products manufactured by Cyberonics, Inc. may be described, for example, in U.S. Pat. Nos. 5,928,272; 5,540,730 and 5,299,569.

Regardless of the specific design features, for vagal nerve stimulation to be effective in arrhythmias, the leads must be accurately positioned adjacent to the left vagus nerve. If excessive scar tissue growth or extracellular matrix deposition occurs around the VNS leads, this can reduce the efficacy of the device. VNS devices that release a therapeutic drug combination, or individual component(s) thereof, able to reducing scarring at the electrode-tissue interface can increase the efficiency of impulse transmission and increase the duration that these devices function clinically. In one aspect, the device includes VNS devices and/or leads that are coated with an anti-scarring drug combination, or individual component(s) thereof, or a composition that includes an anti-scarring drug combination, or individual component(s) thereof. As an alternative to this, or in addition to this, a composition that includes an anti-scarring drug combination, or individual component(s) thereof, can be infiltrated into the tissue surrounding the vagus nerve where the lead will be implanted.

Although numerous cardiac rhythm management (CRM) devices have been described above, all possess similar design features and cause similar unwanted fibrous tissue reactions following implantation. The CRM device, particularly the lead(s), must be positioned in a very precise manner to ensure that stimulation is delivered to the correct anatomical location within the atrium and/or ventricle. All, or parts, of a CRM device can migrate following surgery, or excessive scar tissue growth can occur around the implant, which can lead to a reduction in the performance of these devices. CRM devices that release a therapeutic drug combination, or individual component(s) thereof, for reducing scarring at the electrode-tissue interface can be used to increase the efficacy and/or the duration of activity of the implant (particularly for fully-implanted, battery-powered devices). In one aspect, the present invention provides CRM devices that include a fibrosis-inhibiting drug combination, or individual component(s) thereof, or a composition that includes a fibrosis-inhibiting drug combination, or individual component(s) thereof. Numerous polymeric and non-polymeric delivery systems for use in CRM devices have been described above. These compositions can further include one or more fibrosis-inhibiting drug combinations, or individual component(s) thereof, such that the overgrowth of granulation or fibrous tissue is inhibited or reduced.

Methods for incorporating fibrosis-inhibiting compositions onto or into CRM devices include: (a) directly affixing to the CRM device, lead and/or electrode a fibrosis-inhibiting composition (e.g., by either a spraying process or dipping process as described above, with or without a carrier), (b) directly incorporating into the CRM device, lead and/or electrode a fibrosis-inhibiting composition (e.g., by either a spraying process or dipping process as described above, with or without a carrier (c) by coating the CRM device, lead and/or electrode with a substance such as a hydrogel which will in turn absorb the fibrosis-inhibiting composition, (d) by interweaving fibrosis-inhibiting composition coated thread (or the polymer itself formed into a thread) into the device, lead and/or electrode structure, (e) by inserting the CRM device, lead and/or electrode into a sleeve or mesh which is comprised of, or coated with, a fibrosis-inhibiting composition, (f) constructing the CRM device, lead and/or electrode itself (or a portion of the lead and/or electrode) with a fibrosis-inhibiting composition, or (g) by covalently binding the fibrosis-inhibiting drug combination, or individual component(s) thereof, directly to the CRM device, lead and/or electrode surface, or to a linker (small molecule or polymer) that is coated or attached to the device, lead and/or electrode surface. Each of these methods illustrates an approach for combining an electrical device with a fibrosis-inhibiting (also referred to herein as an anti-scarring) or gliosis-inhibiting drug combination, or individual component(s) thereof, according to the present invention.

For CRM devices, leads and electrodes, the coating process can be performed in such a manner as to: (a) coat the non-electrode portions of the lead; (b) coat the electrode portion of the lead; or (c) coat all or parts of the entire device with the fibrosis-inhibiting composition. In addition to, or alternatively, the fibrosis-inhibiting drug combination, or individual component(s) thereof, can be mixed with the materials that are used to make the CRM device, lead and/or electrode such that the fibrosis-inhibiting drug combination, or individual component(s) thereof, is incorporated into the final product. In these manners, a medical device may be prepared which has a coating, where the coating is, e.g., uniform, non-uniform, continuous, discontinuous, or patterned.

In another aspect, a CRM device may include a plurality of reservoirs within its structure, each reservoir configured to house and protect a therapeutic drug. The reservoirs may be formed from divets in the device surface or micropores or channels in the device body. In one aspect, the reservoirs are formed from voids in the structure of the device. The reservoirs may house a single drug combination, or individual component(s) thereof, or more than one drug combination, or individual component(s) thereof. The drug combination(s), or individual component(s) thereof, may be formulated with a carrier (e.g., a polymeric or non-polymeric material) that is loaded into the reservoirs. The filled reservoir can function as a drug delivery depot which can release drug combination, or individual component(s) thereof, over a period of time dependent on the release kinetics of the drug(s) from the carrier. In certain embodiments, the reservoir may be loaded with a plurality of layers. Each layer may include a different drug combination, or individual component(s) thereof, having a particular amount (dose) of drug combination, or individual component(s) thereof, and each layer may have a different composition to further tailor the amount of drug combination, or individual component(s) thereof, that is released from the substrate. The multi-layered carrier may further include a barrier layer that prevents release of the drug(s). The barrier layer can be used, for example, to control the direction that the drug combination, or individual component(s) thereof, elutes from the void. Thus, the coating of the medical device may directly contact the electrical device, or it may indirectly contact the electrical device when there is something, e.g., a polymer layer, that is interposed between the electrical device and the coating that contains the fibrosis-inhibiting drug combination, or individual component(s) thereof.

In addition to, or as an alternative to incorporating a fibrosis-inhibiting drug combination, or individual component(s) thereof, onto, or into, the CRM device, the fibrosis-inhibiting drug combination, or individual component(s) thereof, can be applied directly or indirectly to the tissue adjacent to the CRM device (preferably near the electrode-tissue interface). This can be accomplished by applying the fibrosis-inhibiting drug combination, or individual component(s) thereof, with or without a polymeric, non-polymeric, or secondary carrier: (a) to the lead and/or electrode surface (e.g., as an injectable, paste, gel, or mesh) during the implantation procedure; (b) to the surface of the tissue (e.g., as an injectable, paste, gel, in situ forming gel, or mesh) prior to, immediately prior to, or during, implantation of the CRM device and/or the lead; (c) to the surface of the CRM lead and/or electrode and/or to the tissue surrounding the implanted lead or electrode (e.g., as an injectable, paste, gel, in situ forming gel, or mesh) immediately after the implantation of the CRM device, lead and/or electrode; (d) by topical application of the anti-fibrosis drug combination, or individual component(s) thereof, into the anatomical space where the CRM device, lead and/or electrode will be placed (particularly useful for this embodiment is the use of polymeric carriers which release the fibrosis-inhibiting drug combination, or individual component(s) thereof, over a period ranging from several hours to several weeks—fluids, suspensions, emulsions, microemulsions, microspheres, pastes, gels, microparticulates, sprays, aerosols, solid implants and other formulations which release the drug combination, or individual component(s) thereof, can be delivered into the region where the CRM device, lead and/or electrode will be inserted); (e) via percutaneous injection into the tissue surrounding the CRM device, lead and/or electrode as a solution, as an infusate, or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

It should be noted that certain polymeric carriers themselves can help prevent the formation of fibrous tissue around the CRM lead and electrode. These carriers (to be described shortly) are particularly useful for the practice of this embodiment, either alone, or in combination with a fibrosis-inhibiting composition. The following polymeric carriers can be infiltrated (as described in the previous paragraph) into the vicinity of the CRM device, lead and/or electrode-tissue interface and include: (a) sprayable collagen-containing formulations such as COSTASIS and CT3, either alone, or loaded with a fibrosis-inhibiting drug combination, or individual component(s) thereof, applied to the implantation site (or the implant/device surface); (b) sprayable PEG-containing formulations such as COSEAL, FOCALSEAL, SPRAYGEL or DURASEAL, either alone, or loaded with a fibrosis-inhibiting drug combination, or individual component(s) thereof, applied to the implantation site (or the implant/device surface); (c) fibrinogen-containing formulations such as FLOSEAL or TISSEAL, either alone, or loaded with a fibrosis-inhibiting drug combination, or individual component(s) thereof, applied to the implantation site (or the implant/device surface); (d) hyaluronic acid-containing formulations such as RESTYLANE, HYLAFORM, PERLANE, SYNVISC, SEPRAFILM, SEPRACOAT, loaded with a fibrosis-inhibiting drug combination, or individual component(s) thereof, applied to the implantation site (or the implant/device surface); (e) polymeric gels for surgical implantation such as REPEL or FLOWGEL loaded with a fibrosis-inhibiting drug combination, or individual component(s) thereof, applied to the implantation site (or the implant/device surface); (f) orthopedic “cements” used to hold prostheses and tissues in place loaded with a fibrosis-inhibiting drug combination, or individual component(s) thereof, applied to the implantation site (or the implant/device surface), such as OSTEOBOND, low viscosity cement (LVC), SIMPLEX P, PALACOS, and ENDURANCE; (g) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT, either alone, or loaded with a fibrosis-inhibiting drug combination, or individual component(s) thereof, applied to the implantation site (or the implant/device surface); (h) implants containing hydroxyapatite [or synthetic bone material such as calcium sulfate, VITOSS and CORTOSS (Orthovita)] loaded with a fibrosis-inhibiting drug combination, or individual component(s) thereof, applied to the implantation site (or the implant/device surface); (i) other biocompatible tissue fillers loaded with a fibrosis-inhibiting drug combination, or individual component(s) thereof, such as those made by BioCure, Inc., 3M Company and Neomend, Inc., applied to the implantation site (or the implant/device surface); (j) polysaccharide gels such as the ADCON series of gels either alone, or loaded with a fibrosis-inhibiting drug combination, or individual component(s) thereof, applied to the implantation site (or the implant/device surface); and/or (k) films, sponges or meshes such as INTERCEED, VICRYL mesh, and GELFOAM loaded with a fibrosis-inhibiting drug combination, or individual component(s) thereof, applied to the implantation site (or the implant/device surface).

A preferred polymeric matrix which can be used to help prevent the formation of fibrous or gliotic tissue around the CRM lead and electrode, either alone or in combination with a fibrosis-inhibiting (or gliosis-inhibiting) drug combination, or individual component(s) thereof, or a composition comprising a fibrosis-inhibiting (or gliosis-inhibiting) drug combination, or individual component(s) thereof, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic drug combination, or individual component(s) thereof, or a stand-alone composition to help prevent the formation of fibrous or gliotic tissue around the CRM lead and electrode.

It should be apparent to one of skill in the art that potentially any anti-scarring drug combination, or individual component(s) thereof, described herein may be utilized alone, or in combination, in the practice of this embodiment. As CRM devices, leads and electrodes are made in a variety of configurations and sizes, the exact dose administered may vary with device size, surface area and design. However, certain principles can be applied in the application of this art. Drug dose can be calculated as a function of dose per unit area (of the portion of the device being coated), total drug dose administered can be measured, and appropriate surface concentrations of active drug can be determined. Regardless of the method of application of the drug(s) to the device (i.e., as a coating or infiltrated into the surrounding tissue), the anti-scarring drug combination(s), or individual component(s) thereof, used alone or in combination, may be administered under the following dosing guidelines:

Drugs and dosage: Exemplary anti-scarring drug combinations that may be used include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for cardiac rhythm management devices will depend on a variety of factors, including the type of formulation, the location of the treatment site, and the type of condition being treated, as well as the surface area of the device. However, certain principles can be applied in the application of this art. Drug dose can be calculated as a function of dose per unit area (of the treatment site), wherein the total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring or anti-gliosis drug combination, or individual component(s) thereof, is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In certain embodiments, the drug is released in effective concentrations for a period ranging from 1-90 days. It should be understood in certain embodiments that within the drug combination, one drug may be released at a different rate and/or for a different amount of time than the other drug(s).

The exemplary anti-fibrosing drug combinations, or individual components thereof, should be administered under the following dosing guidelines. The total amount (dose) of anti-scarring agent(s) in the drug combinations or compositions that comprise the drug combinations can be in the range of about 0.01 μg-10 μg, or 10 μg-100 μg, or 100 μg-1000 μg, or 1 mg-10 mg, or 10 mg-250 mg, or 250 mg-1000 mg, or 1000 mg-2500 mg. The dose (amount) of anti-scarring or anti-gliosis agent(s) per unit area of surface to which the agent is applied may be in the range of about 0.01 μg/mm²-1 μg/mm², or 1 μg/mm²-10 μg/mm², or 10 μg/mm²-250 μg/mm², or 250 μg/mm²-1000 g/mm², or 1000 μg/mm²-2500 μg/mm².

Provided below are exemplary drug combinations and dosage ranges for various anti-scarring and/or anti-gliosis drug combinations or individual components thereof that can be used in conjunction with cardiac rhythm management devices in accordance with the invention.

Exemplary anti-fibrotic drug combinations for description of dosing include, but are not limited to amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof: total dose of each drug within the combination not to exceed 1,500 mg (range of 0.1 μg to 1,500 mg; preferred 1 μg to 1000 mg). Dose per unit area of 0.01 μg/mm² to 200 μg/mm²; preferred dose of 0.1 μg/mm² to 100 μg/mm². Minimum concentration of 10⁻⁸ to 10⁻⁴ M of agent is to be maintained on the implant or barrier surface. Molar ratio of each drug in the combination is to be within the range of 1:1 to 1:1000. Molar ratios within this range may include but are not limited to 1:5, 1:10, 1:15, 1:20, 1:30, 1:50, 1:75, 1:100, 1:200, 1:500, 1:1000. Note that molar ratios may also lie between the ratios stated above.

Therapeutic Drug Combinations for Use with Electrical Medical Devices and Implants

In one aspect, the present application provides various anti-scarring (anti-fibrosis or anti-gliosis) drug combinations. In certain embodiments, one therapeutic agent of an anti-scarring drug combination enhances the anti-scarring activities of the other therapeutic agent(s) in the combination. In certain embodiments, each of the therapeutic agents of an anti-scarring drug combination has anti-scarring activities. In certain embodiments, one therapeutic agent in an anti-scarring drug combination produces a synergistic anti-scarring effect with the other therapeutic agent(s) in an anti-scarring drug combination.

In certain embodiments, individual therapeutic agents in the anti-scarring drug combinations of the present invention may be an antidepressant, steroid, anti-platelet agent, anti-fungal agent, prostaglandin, phosphodiesterase IV inhibitor, antihistamine agent, HMG-CoA reductase inhibitor, metal ion, osmotic laxative, selective serotonin reuptake inhibitor (SSRI), vasodilator, antipsychotic, ophthalmic, anti-mycotic agent, mucosal or dental anesthetic, dopaminergic agent, anti-protozoal, antiestrogen, maradrenaline reuptake inhibitor, non-steroidal immunophilin-dependent immunosuppressant (NSIDI), non-steroidal immunophilin-dependent immunosuppressant enhancer (NSIDIE), antihelmintic drug, antiproliferative agent, antiarrhythmic agent, phenothiazine conjugate, kinesin inhibitor, agent that reduces the biological activity for a mitotic kinesin, or agent that reduces the biological activity of protein tyrosine phosphatase.

In certain embodiments, the anti-scarring drug combinations of the present invention comprise two therapeutic agents that themselves have anti-scarring activities or that enhance the anti-scarring activities of other agents. In certain embodiments, the anti-scarring drug combinations of the present invention comprise three, four, five or more such therapeutic agents.

In certain embodiments, the therapeutic drug combinations of the present invention are suitable to inhibit fibrous (or glial) tissue accumulation around the device bodies, leads and electrodes of implantable electrical devices, e.g., neurostimulation and cardiac rhythm management devices. In certain embodiments, the invention provides for devices that include a drug combination that inhibits this tissue accumulation in the vicinity of the device, i.e., between the medical device and the host into which the medical device is implanted. The drug combination is therefore effective for this goal, is present in an amount that is effective to achieve this goal, and is present at one or more locations that allow for this goal to be achieved. The device is designed to allow the beneficial effects of the drug combination to occur. Also, these therapeutic drug combinations, or individual component(s) thereof, can be used alone, or in combination, to prevent scar (or glial) tissue build-up in the vicinity of the electrode-tissue interface in order to improve the clinical performance and longevity of these implants.

Suitable fibrosis-inhibiting or gliosis-inhibiting drug combinations, or individual component(s) thereof, may be readily identified based upon in vitro and in vivo (animal) models, such as those provided in Examples 37-50. Agents which inhibit fibrosis (or gliosis) can also be identified through in vivo models including inhibition of intimal hyperplasia development in the rat balloon carotid artery model (Examples 42 and 50). The assays set forth in Examples 41 and 49 may be used to determine whether an agent is able to inhibit cell proliferation in fibroblasts and/or smooth muscle cells. In one aspect of the invention, the agent has an IC₅₀ for inhibition of cell proliferation within a range of about 10⁻⁶ to about 10⁻¹⁰ M. The assay set forth in Example 45 may be used to determine whether an agent may inhibit migration of fibroblasts and/or smooth muscle cells. In one aspect of the invention, the agent has an IC₅₀ for inhibition of cell migration within a range of about 10⁻⁶ to about 10⁻⁹M. Assays set forth herein may be used to determine whether an agent is able to inhibit inflammatory processes, including nitric oxide production in macrophages (Example 37), and/or TNF-alpha production by macrophages (Example 38), and/or IL-1 beta production by macrophages (Example 46), and/or IL-8 production by macrophages (Example 47), and/or inhibition of MCP-1 by macrophages (Example 48). In one aspect of the invention, the agent has an IC₅₀ for inhibition of any one of these inflammatory processes within a range of about 10⁻⁶ to about 10⁻¹⁰M. The assay set forth in Example 43 may be used to determine whether an agent is able to inhibit MMP production. In one aspect of the invention, the agent has an IC₅₀ for inhibition of MMP production within a range of about 10⁻⁴ to about 10⁻⁸M. The assay set forth in Example 44 (also known as the CAM assay) may be used to determine whether an agent is able to inhibit angiogenesis. In one aspect of the invention, the agent has an IC₅₀ for inhibition of angiogenesis within a range of about 10⁻⁶ to about 10⁻¹⁰M. Agents which reduce the formation of surgical adhesions may be identified through in vivo models including the rabbit surgical adhesions model (Example 40) and the rat caecal sidewall model (Example 39). These pharmacologically active agents (described below) can then be delivered at appropriate dosages (described herein) into the tissue either alone, or via carriers (formulations described herein), to treat the clinical problems described herein.

Compounds useful in the invention include those described herein in any of their pharmaceutically acceptable forms, including isomers such as diastereomers and enantiomers, salts, solvates, and polymorphs, thereof, as well as racemic mixtures of the compounds described herein. Structural or functional analogs or metabolites of these compounds may also be used.

In certain embodiments, one or more of the components of the drug combinations of the present invention are approved by a national pharmaceutical regulatory agency, such as the United States Food and Drug Administration (USFDA) for administration to a human.

Certain exemplary drug combinations described below are also described in the following publications of U.S. and PCT patent applications (which are incorporated in their entireties by reference): WO 02/58697, WO 03/06026, WO 03/30823, WO 03/57162, WO 03/66049, WO 03/03580, WO 03/92617, WO 04/002430, WO 04/007676, WO 04/006906, WO 02/006842, WO 04/006849, WO 04/030618, US 2004/157837, WO 04/073631, WO 04/073614, WO 05/011572, WO 04/105696, WO 05/000208, WO 05/027839, WO 05/020913, WO 05/027842, WO 05/048927, WO 05/053613, and WO 05/046607.

Exemplary classes of drug combinations are provided below. For each class of drug combinations, the present invention includes each combination of individual components described herein that has anti-scarring activity.

Combinations Comprising Amoxapine and Prednisolone

In certain embodiments, the drug combination according to the present invention comprises amoxapine (an antidepressant) and prednisolone (a steroid).

Prednisolone has the following structure:

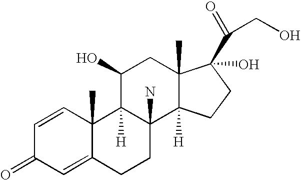

Amoxapine has the following structure:

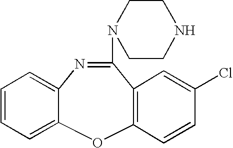

This drug combination is in clinical phase IIa trials in the United States.

Preclinical data suggest that when administered together, amoxapine synergistically increases the immuno-modulatory activity of the reduced-dose steroid without a comparable increase in its adverse side effects, indicating that this drug combination may have a superior risk-to-benefit ratio compared to traditional steroids.

In vitro, this drug combination synergistically inhibits TNF-α release from stimulated primary human lymphocytes as measured by Loewe and other standard synergy models. It also synergistically inhibits IFN-γ and IL-2 in vitro. Although not wishing to be bound by any particular theories, it is believed that the increased activity of the reduced-dose steroid in this drug combination occurs in part through action involving T-cells.

The mechanism studies of this drug combination show amoxapine does not promote glucocorticoid receptor trafficking and does not potentiate prednisolone's ability to transactivate a transfected GRE reporter plasmid in T cells. Amoxapine is observed to block NFAT activation, translocation and transactivation, effects not observed with prednisolone. Amoxapine partially inhibits NFkB and AP1 activation (at low potency), an effect also observed with prednisolone. Inhibition of p38 and JNK activation by amoxapine is observed, whereas ERK is unaffected. These data support a mechanistic model in which amoxapine plays a synergistic immuno-modulatory role in this drug combination by selectively enhancing a subset of prednisolone's actions on pathways of T cell activation.

In both acute and chronic in vivo models of inflammation, amoxapine alone and reduced dose prednisolone alone produced modest or no benefit. However, in the acute model, this drug combination potently inhibited TNF-α production (>50%) similar to a 100-fold higher dose of prednisolone alone (61%). In the chronic model, daily oral dosing of this drug combination significantly inhibited joint swelling by 64%, an inhibition equivalent to a >10-fold higher dose of prednisolone (51%) alone. Chronic treatment with this drug combination did not recapitulate the steroid toxicities on body and organ weight, blood glucose, and HPA suppression observed with high dose steroid treatment.

Combination Comprising Paroxetine and Prednisolone

In certain embodiments, the drug combination according to the present invention comprises paroxetine (a selective serotonin reuptake inhibitor (SSRI)) and prednisolone (a steroid)

The structure of prednisolone is shown above. The structure of paroxetine is shown below:

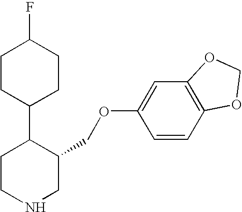

This drug combination is in clinical phase IIa trials in Europe and Canada.

Preclinical data suggest that when administered together, paroxetine synergistically increases the immuno-modulatory activity of a reduced-dose of prednisolone without a comparable increase in its adverse side effects, indicating that this drug combination may have a superior risk-to-benefit ratio compared to traditional steroids.

This drug combination elicits synergistic immuno-modulatory effects without potentiating steroid-associated side effects, and does so through paroxetine's action on key signaling pathways in activated T cells distinct from and synergistic with those affected by prednisolone. It synergistically inhibits multiple cytokines, including TNF-α, IFN-γ and IL-2, released from stimulated primary human lymphocytes.

Due to the mechanism of synergy of this drug combination, paroxetine does not promote glucocorticoid receptor trafficking or potentiate prednisolone's ability to transactivate a GRE reporter plasmid T cells. Paroxetine represses NFAT activation, translocation and transactivation and inhibits NFkB and AP 1 activation through inhibition of p38 and JNK but not ERK activation.

In an in vivo LPS-induced TNF-α release model, this drug combination inhibits TNF-α production by 51% when given 2 hours prior to LPS treatment. This effect was similar to a 100× higher dose of prednisolone alone. The anti-inflammatory effect in vivo was not accompanied by potentiation of steroid side effects such as HPA suppression.

This drug combination has been tested in a human pharmacology endotoxemia study, an acute model of inflammatory markers. In the study, this drug combination inhibited certain pro-inflammatory biomarkers, such as TNF-alpha, IL-6, and C-reactive protein and increased the anti-inflammatory cytokine IL-10.

Combination Comprising Dipyridamole and Prednisolone

In certain embodiments, the drug combination according to the present invention comprises dipyridamole (an anti-platelet agent) and prednisolone (a steroid).

The structure of prednisolone is shown above. The structure of dipyridamole is shown below:

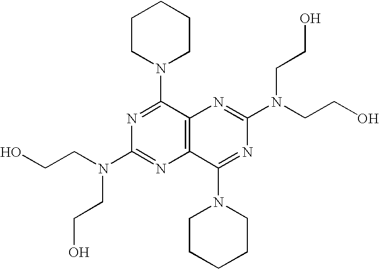

This drug combination is in clinical phase II trials in Europe.

Preclinical data suggest that when administered together, dipyridamole synergistically increases the immuno-modulatory activity of the reduced-dose prednisolone without a comparable increase in its adverse side effects, indicating that this may have a superior risk-to-benefit ratio compared to traditional steroids.

In vitro, this drug combination synergistically inhibits TNF-α release from stimulated primary human lymphocytes as measured by Loewe and other standard synergy models. This drug combination also synergistically inhibits IFN-γ in vitro. Although not wishing to be bound by any particular theories, it is believed that the increased activity of the reduced-dose steroid in this drug combination occurs in part through an action involving macrophages, which are important components of the immune system.

In vivo, a single p.o. dose of this drug combination potently inhibited LPS-induced TNF-α production by 72%. In the adjuvant model, this drug combination inhibited joint swelling by 54% while in the CIA model the drug combination reduced the arthritis severity score by 58%, compared to vehicle controls. In each model, the components of this drug combination had little or no activity. Further, the effect of this drug combination in these models was similar to that seen with ≧10 fold higher steroid doses. Chronic treatment with this drug combination did not recapitulate the steroid toxicities on body weight, glucose utilization and HPA suppression observed with high dose steroid treatment.

Combination Comprising Dexamethasone and Econazole

In certain embodiments, the drug combination according to the present invention comprises dexamethasone (a steroid) and econazole (an anti-fungal agent).

The structure of dexamethasone is shown below:

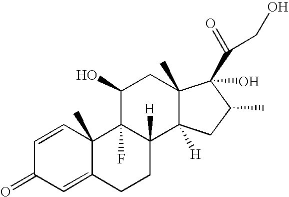

The structure of econazole nitrate is shown below:

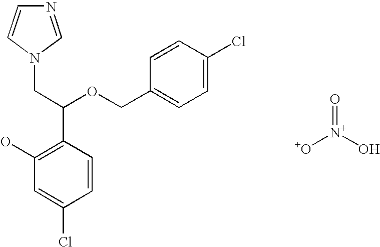

This drug combination is a research phase combination that has not yet entered preclinical studies. In vitro studies show this drug combination synergistically inhibits the production of TNF-α.

Combination Comprising Diflorasone and Alprostadil

In certain embodiments, the drug combination according to the present invention comprises diflorasone (a steroid) and alprostadil (a prostaglandin).

The structure of diflorasone is shown below:

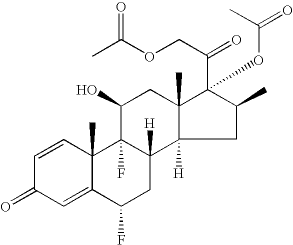

The structure of prostaglandin E is shown below:

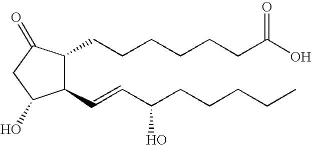

This drug combination synergistically inhibits multiple cytokines including TNF-α released from LPS-stimulated human peripheral mononuclear blood cells.

Combination Comprising Dipyridamole and Amoxapine

In certain embodiments, the drug combination of the present invention comprises dipyridamole (a cardiovascular drug; an anti-platelet agent) and amoxapine (an anti-depressant).

The structures of dipyridamole and amoxapine are shown above.

This drug combination is in clinical phase IIa trials in Europe.

This drug combination is an orally administered synergistic cytokine modulator that combines two active pharmaceutical ingredients, neither of which is indicated for the treatment of immuno-inflammatory disease. When administered together, these active pharmaceutical ingredients show the potential in preclinical studies to synergistically inhibit important disease-relevant cytokines, including the cytokine TNF-alpha.

This drug combination synergistically inhibits multiple cytokines including TNF-α released from LPS-stimulated human peripheral mononuclear blood cells. This affect was confirmed in the acute in vivo LPS model where the drug combination significantly inhibited TNF-α release (>75%). This effect was similar to a high dose of prednisolone (10 mg/Kg). The components of this drug combination had no significant effect in the in vivo TNF-α release studies. In the chronic arthritis model, daily oral dosing of this drug combination significantly inhibited joint swelling by >40%. The components of this drug combination had minimal effects in this model. Furthermore, chronic treatment with this drug combination or its components elicited minimal effects on body and organ weight, blood glucose, and HPA suppression.

Combination Comprising Dipyridamole and Ibudilast

In certain embodiments, the drug combination of the present invention comprises dipyridamole (an anti-platelet agent) and ibudilast (a phosphodiesterase IV inhibitor).

The structure of ibudilast is shown below, while the structure of dipyridamole is shown above.

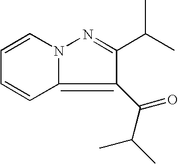

It synergistically inhibits TNF-α released from LPS-stimulated human peripheral mononuclear blood cells.

Combination Comprising Nortriptyline and Loratadine (or Desloratadine)

In certain embodiments, the drug combination according to the present invention comprises nortriptyline (a tricyclic anti-depressant agent) and loratadine (or desloratadine) (an antihistamine).

The structure of nortriptyline hydrochloride is shown below:

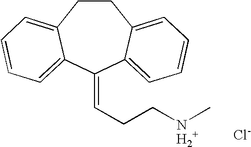

The structure of loratadine is shown below:

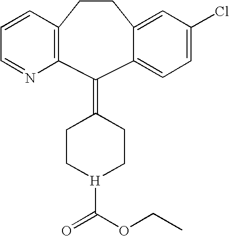

This drug combination has shown potent synergistic inhibition of TNF-α and other pro-inflammatory cytokines in in vitro studies. In addition, loratadine inhibits mast cells and eosinophil activation.

Combination Comprising Albendazole and Pentamidine

In certain embodiments, the drug combination according to the present invention comprises albendazole and pentamidine.

The structure of albendazole is shown below:

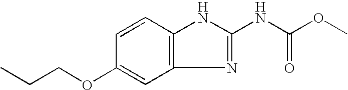

The structure of pentamidine is shown below:

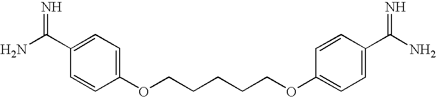

This drug combination is at a pre-clinical phase of development.

This drug combination synergistically inhibits the proliferation of A549 cells in vitro. It has demonstrated potent, highly synergistic anti-tumor effects in animal models of NSCLC. The anti-tumor effects of this drug combination are dose dependent and comparable to the activity of gold standard antineoplastics without the associated toxicities.

Combination Comprising Itraconazole and Lovastatin

In certain embodiments, the drug combination according to the present invention comprise itraconazole (an anti-fungal agent) and lovastatin (an HMG-CoA reductase inhibitor).

The structure of itraconazole is shown below:

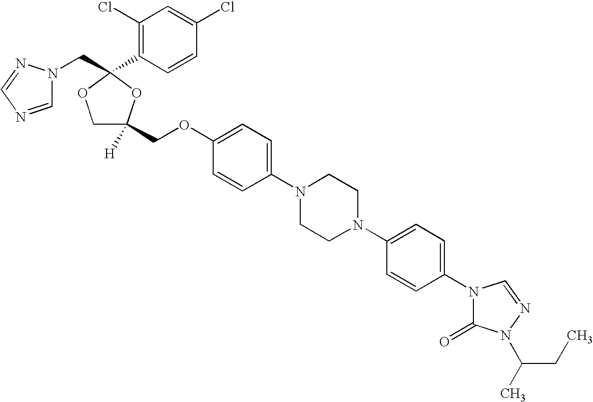

The structure of lovastatin is shown below:

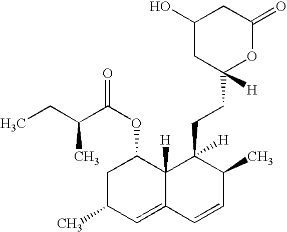

This drug combination demonstrates highly synergistic inhibition of the proliferation of multiple cancer cell lines in vitro, including A549 (NSCLC), PANC-1 (Pancreatic), HCT-116 (Colorectal), DU-145 (Prostate), and SKMEL28 (Melanoma). It has potential application to multiple proliferative diseases.

Combination Comprising Terbinafine and Manganese Sulfate

In certain embodiments, the drug combination according to the present invention comprises terbinafine (an anti-fungal agent) and manganese sulfate (to provide a metal ion).

The structure of terbinafine hydrochloride is shown below:

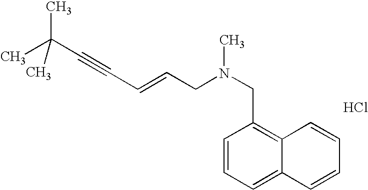

The structure of manganese sulfate is shown below:

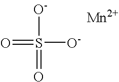

Manganese ion synergistically potentiates the anti-fungal activity of terbinafine against multiple drug-resistant strains of C. glabrata.

Drug Combination Comprising a Tricyclic Compound and a Steroid

In certain embodiments, the drug combination that has anti-scarring activity comprises at least two agents, wherein at least one agent is a tricyclic compound, such as a tricyclic antidepressant (TCA) and at least one second agent is a steroid such as a corticosteroid. Examples of drug combinations include a drug combination that comprises at least two agents in amounts that together may be sufficient to alter the immune response, that is, the at least two agents alone or in combination reduce or inhibit an immune response by a host or subject (or patient), including inhibiting or reducing inflammation (an inflammatory response) and/or an autoimmune response.

The drug combination may further comprise one or more additional compounds (e.g., a glucocorticoid receptor modulator, NSAID, COX-2 inhibitor, DMARD, biologic, small molecule immunomodulator, xanthine, anticholinergic compound, beta receptor agonist, bronchodilator, non-steroidal immunophilin-dependent immunosuppressant, vitamin D analog, psoralen, retinoid, or 5-amino salicylic acid). The composition may be formulated, for example, for topical administration or systemic administration.

Compounds useful in the drug combinations include those described herein in any of their pharmaceutically acceptable forms, including isomers such as diastereomers and enantiomers, salts, solvates, and polymorphs thereof, as well as racemic mixtures of the compounds described herein.

In the generic descriptions of compounds described herein, the number of atoms of a particular type in a substitutent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 7 carbon atoms in addition to one or more heteroatoms. Other numbers of atoms and other types of atoms may be indicated in a similar manner.

The term “pharmaceutically active salt” refers to a salt that retains the pharmaceutical activity of its parent compound.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “fexofenadine” is meant the free base, as well as any pharmaceutically acceptable salt thereof (e.g., fexofenadine hydrochloride).

Tricyclic Compound

By “tricyclic compound” is meant a compound having one of formulas (I), (II), (III), or (IV): 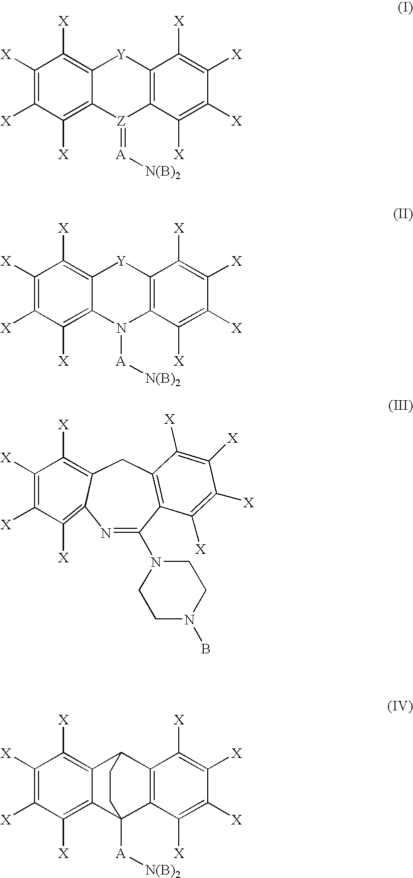
wherein each X is, independently, H, Cl, F, Br, I, CH₃, CF₃, OH, OCH₃, CH₂CH₃, or OCH₂CH₃; Y is CH₂, O, NH, S(O)₀₋₂, (CH₂)₃, (CH)₂, CH₂O, CH₂NH, CHN, or CH₂S; Z is C or S; A is a branched or unbranched, saturated or monounsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, or S(O)₀₋₂. In preferred embodiments, each X is, independently, H, Cl, or F; Y is (CH₂)₂, Z is C; A is (CH₂)₃; and each B is, independently, H, Cl, or F.

Tricyclic compounds include tricyclic antidepressants such as amoxapine, 8-hydroxyamoxapine, 7-hydroxyamoxapine, loxapine (e.g., loxapine succinate, loxapine hydrochloride), 8-hydroxyloxapine, amitriptyline, clomipramine, doxepin, imipramine, trimipramine, desipramine, nortriptyline, and protriptyline, although compounds need not have antidepressant activities to be considered tricyclic compounds as described herein.

Tricyclic compounds include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo[b,e)(1,4)diazepine; 5,10-dihydro-7-chloro-10-(2-(morpholino)ethyl)-11H-dibenzo[b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo[b,e)(1,4)diazepine; 4-(11H-dibenz[b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. patent application Ser. Nos. 10/617,424 or 60/504,310.

Amoxapine

Amoxapine is a tricyclic antidepressant (TCA) of the dibenzoxapine type. It is structurally similar to the older TCAs and also shares similarities with the phenothiazines.

The exact action of TCAs is not fully understood, but it is believed that one of their important effects is the enhancement of the actions of norepinephrine and serotonin by blocking the reuptake of various neurotransmitters at the neuronal membrane. Amoxapine also shares some similarity with antipsychotic drugs in that it blocks dopamine receptors and can cause dyskinesia. Amoxapine also blocks the reuptake of norepinephrine, similar to the action of desipramine and maprotiline.

Based on the ability of amoxapine to act in concert with prednisolone to inhibit TNFα levels, one skilled in the art will recognize that other TCAs, as well as structural and functional analogs of amoxapine, can also be used in combination with prednisolone (or another corticosteroid—see below). Amoxapine analogs include, for example, 8-hydroxyamoxapine, 7-hydroxyamoxapine, loxapine, loxapine succinate, loxapine hydrochloride, 8-hydroxyloxapine, clothiapine, perlapine, fluperlapine, and dibenz(b,f)(1,4)oxazepine, 2-chloro-11-(4-methyl-1-piperazinyl)-, monohydrochloride.

Corticosteroids

By “corticosteroid” is meant any naturally occurring or synthetic compound characterized by a hydrogenated cyclopentanoperhydro-phenanthrene ring system and having immunosuppressive and/or antinflammatory activity. Naturally occurring corticosteriods are generally produced by the adrenal cortex. Synthetic corticosteroids may be halogenated. Functional groups required for activity include a double bond at Δ4, a C3 ketone, and a C20 ketone. Corticosteroids may have glucocorticoid and/or mineralocorticoid activity. Examples of corticosteroids are provided herein.

In one embodiment, at least one (i.e. g, one or more) corticosteroid may be combined and/or formulated with a tricyclic compound in a drug combination described herein. Suitable corticosteroids include 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-[6-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta,21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha,9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; beclomethasone dipropionate monohydrate; betamethasone; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, de hydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; dipropionate; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone; prednisolone 21-hemisuccinate; prednisolone 21-hemisuccinate free acid; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Prednisolone

Prednisolone, a synthetic adrenal corticosteroid, has anti-inflammatory properties, and is used in a wide variety of inflammatory conditions. It is desirable to reduce the amount of administered prednisolone because long-term use of steroids at can produce significant side effects.

Prednisolone is a member of the corticosteroid family of steroids. Based on the shared structural features and apparent mechanism of action among the corticosteroid family, one skilled in the art will recognize that other corticosteroids can be used in combination with amoxapine or an amoxapine analog to treat inflammatory disorders. Corticosteroids include, for example, the compounds listed herein.

The compounds described herein are also useful when formulated as salts. For example, amytriptiline, another tricyclic compound, has been formulated as a hydrochloride salt, indicating that amoxapine can be similarly formulated. Prednisolone salts include, for example, prednisolone 21-hemisuccinate sodium salt and prednisolone 21-phosphate disodium salt.

Other Compounds

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Exemplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharmaceuticals).

Steroid Receptor Modulators

Steroid receptor modulators (e.g., antagonists and agonists) may be used as a substitute for or in addition to a corticosteroid in the drug combinations described herein. Thus, in one embodiment, the drug combination features the combination of a tricyclic compound and a glucocorticoid receptor modulator or other steroid receptor modulator.

Glucocorticoid receptor modulators that may used in the drug combinations described herein include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other compounds that may be used as a substitute for or in addition to a corticosteroid in the drug combinations include, but are not limited to, A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

Non-Steroidal Anti-Inflammatory Drugs (NSAIDs)

In certain embodiments, the tricyclic compound of the drug combination may be administered in conjunction with one or more of non-steroidal anti-inflammatory drugs (NSAIDs), such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin.

When a tricyclic compound is administered in combination with acetylsalicylic acid, the combination may also be effective in modulating an immune response (suppressing TNFα, IL-1, IL-2 or IFN-γ) in vitro. Accordingly, the combination of a tricyclic compound in combination with acetylsalicylic acid and their analogs may be more effective than either agent alone in modulating an immune, particularly an immune response mediated by TNFα, IL-1, IL-2, and/or IFN-γ.

Acetylsalicylic acid, also known by trade name aspirin, is an acetyl derivative of salicylic acid and has the following structural formula.

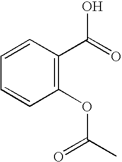

Aspirin is useful in the relief of headache and muscle and joint aches. Aspirin is also effective in reducing fever, inflammation, and swelling and thus has been used for treatment of rheumatoid arthritis, rheumatic fever, and mild infection. Thus in certain embodiments, a drug combination of a tricyclic compound and acetylsalicylic acid (aspirin) or an analog thereof can also be used in the devices and methods described herein.

An NSAID may be administered in conjunction with any one of the drug combinations described herein. For example, a drug combination that includes at least one drug that is also useful for treating and/or preventing an immunological disease or disorder, including an inflammatory disease or disorder, may be a combination of a tricyclic compound and a corticosteroid and further comprising an NSAID, such as acetylsalicylic acid, in conjunction with the combination described above.

Dosage amounts of acetylsalicylic acid are known to those skilled in medical arts, and generally range from about 70 mg to about 350 mg per day. When a lower or a higher dose of aspirin is needed, a formulation containing dipyridamole and aspirin may contain 0-25 mg, 25-50 mg, 50-70 mg, 70-75 mg, 75-80 mg, 80-85 mg, 85-90 mg, 90-95 mg, 95-100 mg, 100-150 mg, 150-160 mg, 160-250 mg, 250-300 mg, 300-350 mg, or 350-1000 mg of aspirin.

When the combinations described herein are used for treatment in conjunction with an NSAID, the dose of the individual components may be reduced substantially to a point below the doses that would be effective for achieving the same effects by administering NSAIDs (e.g., acetylsalicylic acid) or tricyclic compound alone or by administering a combination of an NSAID (e.g., acetylsalicylic acid) and a tricyclic compound. A drug combination that includes a tricyclic compound and an NSAID may have increased effectiveness, safety, tolerability, or satisfaction of treatment of a patient suffering from or at risk of suffering from inflammatory disorder or disease as compared to a composition having a tricyclic compound or an NSAID alone.

Nonsteroidal Immunophilin-Dependent Immunosuppressants

In one embodiment, the drug combination comprises a tricyclic compound and a non-steroidal immunophilin-dependent immunosuppressant (NsIDI), optionally with a corticosteroid or other agent described herein.

By way of background, in healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus) and rapamycin target many types of immunoregulatory cells, including T-cells, and suppress the immune response in organ transplantation and autoimmune disorders.

In one embodiment, the NsIDI is cyclosporine, and in another embodiment, the NsIDI is tacrolimus. In another embodiment, the NsIDI is rapamycin and in still another embodiment, the NsIDI is everolimus. In still other embodiments, the NsIDI is pimecrolimus, or the NsIDI is a calcineurin-binding peptide. Two or more NsIDIs can be administered contemporaneously.

Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g. on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Tacrolimus

Tacrolimus (FK506) is an immunosuppressive agent that targets T cell intracellular signal transduction pathways. Tacrolimus binds to an intracellular protein FK506 binding protein (FKBP-12) that is not structurally related to cyclophilin (Harding et al., Nature 341:758-7601, 1989; Siekienka et al., Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of nuclear factor of activated T cells (NFAT), a nuclear component that initiates gene transcription required for proinflammatory cytokine (e.g., IL-2, gamma interferon) production and T cell activation. Thus, tacrolimus inhibits T cell activation.

Tacrolimus is a macrolide antibiotic that is produced by Streptomyces tsukubaensis. It suppresses the immune system and prolongs the survival of transplanted organs. It is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 0.9% sodium chloride or 5% dextrose prior to injection.

Tacrolimus and tacrolimus analogs are described by Tanaka et al., (J. Am. Chem. Soc., 109:5031, 1987) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

While suggested dosages will vary with a patient's condition, standard recommended dosages are provided below. By way of background, typically patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus

Pimecrolimus, which is described further in detail herein, is the 33-epi-chloro derivative of the macrolactam ascomyin. Pimecrolimus structural and functional analogs are described in U.S. Pat. No. 6,384,073. Pimecrolimus is particularly useful for the treatment of atopic dermatitis.

Rapamycin

Rapamycin is a cyclic lactone produced by Streptomyces hygroscopicus. Rapamycin is an immunosuppressive agent that inhibits T cell activation and proliferation. Like cyclosporines and tacrolimus, rapamycin forms a complex with the immunophilin FKBP-12, but the rapamycin-FKBP-12 complex does not inhibit calcineurin phosphatase activity. The rapamycin immunophilin complex binds to and inhibits the mammalian kinase target of rapamycin (mTOR). mTOR is a kinase that is required for cell-cycle progression. Inhibition of mTOR kinase activity blocks T cell activation and proinflammatory cytokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell 1:627-637, 1998). As a class of calcineurin inhibitors, these agents are useful in the methods of the invention.

As described herein, in one embodiment, a drug combination comprises a tricyclic compound and a corticosteroid. In certain specific embodiments, the drug combination comprises a tricyclic compound wherein the tricyclic compound is a tricyclic antidepressant selected from amoxapine, 8-hydroxyamoxapine, 8-methoxyloxapine, 7-hydroxyamoxapine, loxapine, loxapine succinate, loxapine hydrochloride, 8-hydroxyloxapine, amitriptyline, clomipramine, doxepin, imipramine, trimipramine, desipramine, nortriptyline, maprotiline, norclozapine, olanzapine, or protriptyline. In a specific embodiment, the tricyclic compound is amoxapine.

In a particular embodiment, the tricyclic compound is combined with a corticosteroid wherein the corticosteroid is dexamethasone, betamethasone, triamcinolone, triamcinolone acetonide, triamcinolone diacetate, triamcinolone hexacetonide, beclomethasone, dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone 21-hemisuccinate free acid, prednisolone metasulphobenzoate, prednisolone terbutate, or triamcinolone acetonide 21-palmitate.

In a certain specific embodiment, the corticosteroid is prednisolone. In one embodiment, the drug combination comprises amoxapine and prednisolone. In other specific embodiments, the corticosteroid is prednisolone and the tricyclic compound is protriptyline; in another specific embodiment the corticosteroid is prednisolone and the tricyclic compound is nortriptyline. In other specific embodiments, the drug combination comprises prednisolone and maprotaline. In certain specific embodiments, the corticosteroid is prednisolone and the tricyclic compound is loxapine; the corticosteroid is prednisolone and the tricyclic compound is desipramine; the corticosteroid is prednisolone and the tricyclic compound is clomipramine; the corticosteroid is prednisolone and the tricyclic compound is protriptyline. In another embodiment, the drug combination comprises prednisolone and fluoxotine; in still another embodiment, the drug combination comprises prednisolone and norclozapine.

In other embodiments, the drug combination comprises budesonide and amitriptyline; dexamethasone and amitriptyline; diflorasone and amitriptyline; hydrocortisone and amitriptyline; prednisolone and amitriptyline; triamcinolone and amitriptyline; budesonide and amoxapine; dexamethasone and amoxapine; betamethasone and amoxapine; hydrocortisone and amoxapine; triamcinolone and amoxapine; betamethasone and clomipramine; budesonide and clomipramine; dexamethasone and clomipramine; diflorasone and clomipramine; hydrocortisone and clomipramine; triamcinolone and clomipramine. In other embodiments, the drug combination comprises desipramine with any one of betamethasone, budesonide, dexamethasone, diflorasone, hydrocortisone, prednisolone, and triamcinolone. In still other specific embodiments, the drug combination comprises imipramine with any one of betamethasone, budesonide, dexamethasone, diflorasone, hydrocortisone, prednisolone, and triamcinolone. In another specific embodiment, the drug combination comprises nortriptyline and any one of betamethasone, budesonide, dexamethasone, hydrocortisone, prednisolone, and triamcinolone. In another embodiment, the drug combination comprises protriptyline and any one of betamethasone, budesonide, dexamethasone, diflorasone, hydrocortisone, prednisolone, and triamcinolone.

In another specific embodiment, a structural analog of amoxapine may be used in the drug combination. Such a structural analog may include clothiapine, perlapine, fluperlapine, or dibenz(b,f)(1,4)oxazepine, 2-chloro-11-(4-methyl-1-piperazinyl)-, monohydrochloride, which may be combined with a corticosteroid for use in the devices and methods described herein.

In other certain specific embodiments, the drug combination comprises a tricyclic compound wherein the tricyclic compound is amitriptyline, amoxapine, clomipramine, dothiepin, doxepin, desipramine, imipramine, lofepramine, loxapine, maprotiline, mianserin, mirtazapine, oxaprotiline, nortriptyline, octriptyline, protriptyline, or trimipramine. In a particular embodiment, the tricyclic compound is combined with a corticosteroid, which in certain embodiments is prednisolone, cortisone, budesonide, dexamethasone, hydrocortisone, methylprednisolone, fluticasone, prednisone, triamcinolone, or diflorasone. In a certain specific embodiment, the tricyclic compound is nortriptyline and the corticosteroid is budesonide. The compositions may further comprise an NSAID, COX-2 inhibitor, biologic, DMARD, small molecule immunomodulator, xanthine, anticholinergic compound, beta receptor agonist, bronchodilator, non-steroidal immunophilin-dependent immunosuppressant, vitamin D analog, psoralen, retinoid, or 5-amino salicylic acid. In a specific embodiment, the NSAID is ibuprofen, diclofenac, or naproxen. In another specific embodiment, the COX-2 inhibitor is rofecoxib, celecoxib, valdecoxib, or lumiracoxib. In other certain embodiments, the biologic is adelimumab, etanercept, infliximab, CDP-870, rituximab, or atlizumab; and in other specific embodiments, DMARD is methotrexate or leflunomide; a xanthine is theophylline; a beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline; a non-steroidal immunophilin-dependent immunosuppressant is cyclosporine, tacrolimus, pimecrolimus, or ISAtx247; a vitamin D analog is calcipotriene or calcipotriol; a psoralen is methoxsalen; a retinoid is acitretin or tazoretene; a 5-amino salicylic acid is mesalamine, sulfasalazine, balsalazide disodium, or olsalazine sodium; and a small molecule immunomodulator is VX 702, SCIO 469, doramapimod, RO 30201195, SCIO 323, DPC 333, pranalcasan, mycophenolate, or merimepodib.

Drug Combination Comprising a Tetra-Substituted Pyrimidopyrimidine and a Corticosteroid

In another embodiment, the drug combination that has anti-scarring activity comprises a tetra-substituted pyrimidopyrimidine, such as dipyridamole (also known as 2,6-bis(diethanolamino)-4,8-dipiperidinopyrimido(5,4-d)pyrimidine), and a corticosteroid, such as fludrocortisone (as known as 9-alpha-fluoro-11-beta,17-alpha,21-trihydroxy-4-pregnene-3,20-dione acetate) or prednisolone (also known as 1-dehydrocortisol; 1-dehydrohydrocortisone; 1,4-pregnadiene-11 beta,17alpha,21-triol-3,20-dione; and 11beta,17alpha,21-trihydroxy-1,4-pregnadiene-3,20-dione). At least one biological activity of such agents is the capability to substantially suppress TNFα levels induced in peripheral blood mononuclear cells (PBMCs). Thus, such a drug combination also has the capability to alter the immune response, including inhibiting or reducing inflammation (i.e., an inflammatory response) and/or an autoimmune response.

An exemplary composition comprises (i) a corticosteroid and (ii) a tetra-substituted pyrimidopyrimidine. An exemplary tetra-substituted pyrimidopyrimidine has structure of the formula (V): 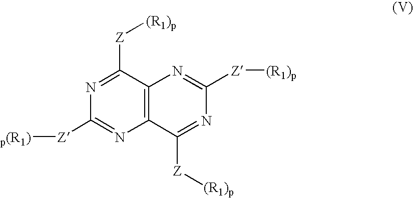
wherein each Z and each Z₁ is, independently, N, O, C,

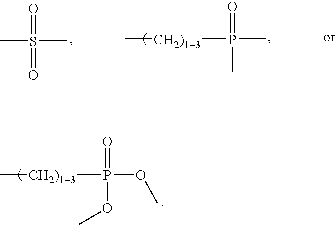

When Z or Z′ is O or 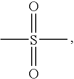
then p=1, when Z or Z′ is N, 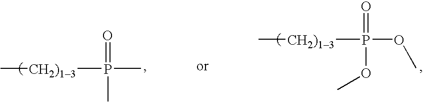
then p=2, and when Z or Z′ is C, then p=3. In formula (V), each R₁ is, independently, X; OH; N-alkyl (wherein the alkyl group has 1 to 20 carbon atoms); a branched or unbranched alkyl group having 1 to 20 carbon atoms; or a heterocycle. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusively. Each Y is, independently, H, F, Cl, Br, or I. In one embodiment, each Z is the same moiety, each Z′ is the same moiety, and Z and Z′ are different moieties. The two compounds are each administered in an amount that, when combined with the second compound, is sufficient to treat or prevent the immunoinflammatory disorder.

The drug combination may also suppress production of one or more proinflammatory cytokines in a host or subject to whom the device is administered, wherein the device comprises an implant and a drug combination as described herein and wherein the drug combination comprises (i) a corticosteroid; and (ii) a tetra-substituted pyrimidopyrimidine having formula (V).

In particularly useful tetra-substituted pyrimidopyrimidines, R₁ is a substituted or unsubstituted furan, purine, or pyrimidine, (CH₂CH₂OY), (CH₂CH(OH)CH₂OY), (HCH₂CH(OH)CX₃), ((CH₂)ₙOY), where n=2-5,

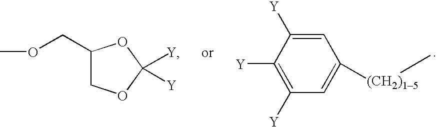

In other useful tetra-substituted pyrimidopyrimidines, each Z is N and the combination of the two associated R₁ groups is —(CH₂)₅—, and each Z′ is N and each associated R₁ group is —CH₂CH₂OH.

The tetra-substituted pyrimidopyrimidine and the corticosteroid may also be combined with a pharmaceutically acceptable carrier, diluent, or excipient.

In certain embodiments, a drug combination comprises one or more tetra-substituted pyrimidopyrimidine compounds and one or more corticosteroid compounds. The drug combination may feature higher order combinations of tetra-substituted pyrimidopyrimidines and corticosteroids. Specifically, one, two, three, or more tetra-substituted pyrimidopyrimidines may be combined with one, two, three, or more corticosteroids. In certain embodiments, the tetra-substituted pyrimidopyrimidine, the corticosteroid, or both are approved by the United States Food and Drug Administration (USFDA) for administration to a human.

Exemplary tetra-substituted pyrimidopyrimidines that may be used in the drug combinations described herein include, for example, 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine), mopidamole, dipyridamole monoacetate, NU3026 (2,6-di-(2,2-dimethyl-1,3-dioxolan-4-yl)-methoxy-4,8-di-piperidinopyrimidopyrimidine), NU3059 (2,6-bis-(2,3-dimethyoxypropoxy)-4,8-d]-piperidinopyrimidopyrimidine), NU3060 (2,6-bis[N,N-di(2-methoxy)ethyl]-4,6-d]-piperidinopyrimidopyrimidine), and NU3076 (2,6-bis(diethanolamino)-4,8-di-4-methoxybenzylaminopyrimidopyrimidine).

Dipyridamole

Dipyridamole (2,6-bis(diethanolamino)-4,8-dipiperidinopyrimido(5,4-d)pyrimidine) is a tetra-substituted pyrimidopyrimidine that is used as a platelet inhibitor, e.g., to prevent blood clot formation following heart valve surgery and to reduced the moribundity associated with clotting disorders, including myocardial and cerebral infarction.

Exemplary tetra-substituted pyrimidopyrimidines are 2,6-disubstituted 4,8-dibenzylaminopyrimido[5,4-d]pyrimidines, including, for example, mopidamole, dipyridamole monoacetate, NU3026 (2,6-di-(2,2-dimethyl-1,3-dioxolan-4-yl)-methoxy-4,8-d]-piperidinopyrimidopyrimidine), NU3059 (2,6-bis-(2,3-dimethyoxypropoxy)-4,8-di-piperidinopyrimidopyrimidine), NU3060 (2,6-bis[N,N-di(2-methoxy)ethyl]-4,6-di-piperidinopyrimidopyrimidine), and NU3076 (2,6-bis(diethanolamino)-4,8-di-4-methoxybenzylaminopyrimido-pyrimidine) (see, e.g., Curtin et al., Br. J. Cancer 80:1738-1746, 1999).

In a particular embodiment, the tetra-substituted pyrimidopyrimidine compound is a 2,6-disubstituted 4,8-dibenzylaminopyrimido[5,4-d]pyrimidine. In another particular embodiment, the compound is dipyridamole, mopidamole, dipyridamole monoacetate, NU3026 (2,6-di-(2,2-dimethyl-1,3-dioxolan-4-yl)-methoxy-4,8-di-piperidinopyrimidopyrimidine), NU3059 (2,6-bis-(2,3-dimethyoxypropoxy)-4,8-di-piperidinopyrimidopyrimidine), NU3060 (2,6-bis[N,N-di(2-methoxy)ethyl]-4,6-di-piperidinopyrimidopyrimidine), or NU3076 (2,6-bis(diethanolamino)-4,8-di-4-methoxybenzylaminopyrimidopyrimidine), and in a specific embodiment, the compound is dipyridamole. In another particular embodiment, tetra-substituted pyrimidopyrimidine compound is a 2,6-disubstituted 4,8-dibenzylaminopyrimido[5,4-d]pyrimidine, and in another particular embodiment, compound is dipyridamole, mopidamole, dipyridamole monoacetate, NU3026, NU3059, NU3060, or NU3076.

Corticosteroids

As described herein, by “corticosteroid” is meant any naturally occurring or synthetic steroid hormone that can be derived from cholesterol and is characterized by a hydrogenated cyclopentanoperhydrophenanthrene ring system. Naturally occurring corticosteroids are generally produced by the adrenal cortex. Synthetic corticosteroids may be halogenated. Functional groups required for activity include a double bond at □4, a C3 ketone, and a C20 ketone. Corticosteroids may have glucocorticoid and/or mineralocorticoid activity. In certain embodiments, the corticosteroid is either fludrocortisone or prednisolone. Additional exemplary corticosteroids are provided in detail herein and are known in the art.

In certain embodiments, the drug combination comprises at least one of the corticosteroids: fludrocortisone (also as known as 9-alpha-fluoro-11-beta,17-alpha,21-trihydroxy-4-pregnene-3,20-dione acetate) and prednisolone (also known as 1-dehydrocortisol; 1-dehydrohydrocortisone; 1,4-pregnadiene-11 beta,17alpha,21-triol-3,20-dione; and 11beta,17alpha,21-trihydroxy-1,4-pregnadiene-3,20-dione); however, a skilled artisan will recognize that structural and functional analogs of these corticosteroids can also be used in combination with the tetra-substituted pyrimidopyrimidines in the methods and compositions described herein. Other useful corticosteroids may be identified based on the shared structural features and apparent mechanism of action among the corticosteroid family. Other exemplary corticosteroids are described in greater detail herein.

Compounds useful in the invention include those described herein in any of their pharmaceutically acceptable forms, including isomers such as diastereomers and enantiomers, salts, solvates, and polymorphs thereof, as well as racemic mixtures of the compounds described herein.

In another embodiment, the corticosteroid is algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-1,7-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-2,1-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, or wortmannin.

By “heterocycle” is meant any cyclic molecule, wherein one or more of the ring atoms is an atom other than carbon. Preferable heterocycles consist of one or two ring structures. Preferable heteroatoms are N, O, and S. Each ring structure of the heterocycle consists of 3-10 atoms, preferably 4-8 atoms, and most preferably 5-7 atoms. Each ring structure need not contain a heteroatom, provided that a heteroatom is present in at least one ring structure. Preferred heterocycles are, for example, beta-lactams, furans, tetrahydrofurans, pyrroles, pyrrolidines, thiophenes, tetrahydrothiophenes, oxazoles, imidazolidine, indole, guanine, and phenothiazine.

By the term “cytokine suppressing amount” is meant an amount of the combination which will cause a decrease in the vivo presence or level of the proinflammatory cytokine, when given to a patient for the prophylaxis or therapeutic treatment of an immunoinflammatory disorder which is exacerbated or caused by excessive or unregulated proinflammatory cytokine production.

The combination of a tetra-substituted pyrimidopyrimidine with a corticosteroid has substantial TNFα suppressing activity against stimulated white blood cells. The combinations of dipyridamole with fludrocortisone, and dipyridamole with prednisolone were particularly effective. Thus, the combination of a tetra-substituted pyrimidopyrimidine with a corticosteroid may also be useful for inhibiting an immune response, particularly an inflammatory response.

In a specific embodiment, the drug combination comprises dipyridamole and fludrocortisone. In another specific embodiment, the drug combination comprises dipyridamole and prednisolone. In yet another specific embodiment, the drug combination comprises dipyridamole and prednisone.

Drug Combination Comprising a Prostaglandin and a Retinoid

In another embodiment, the drug combination that has anti-scarring activity comprises at least two agents wherein at least one agent is a prostaglandin, such as alprostadil (also known as prostaglandin E1; (11α,13E,15S)-11,15-dihydroxy-9-oxoprost-13-enoic acid; 11α,15α-dihydroxy-9-oxo-13-trans-prostenoic acid; or 3-hydroxy-2-(3-hydroxy-1-octenyl)-5-oxocyclopentaneheptanoic acid), and at least one second agent is a retinoid, such as tretinoin (also known as vitamin A; all trans retinoic acid; or 3,7-dimethyl-9-(2,6,6-trimethylcyclohex-1-enyl)nona-2,4,6,8-all-trans-tetraenoic acid). These compounds also exhibit the capability to substantially suppress TNFα levels induced in white blood cells. TNFα is a major mediator of inflammation.

Exemplary prostaglandin compounds include but are not limited to alprostidil, dinoprostone, misoprostil, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, ornoprostil, 13,14-dihydroprostaglandin F2α, and prostaglandin J.

By “retinoid” is meant retinoic acid, retinol, and retinal, and natural or synthetic derivatives of retinoic acid, retinol, or retinal that are capable of binding to a retinoid receptor and consist of four isoprenoid units joined in a head-to-tail manner. Examples of retinoids include tretinoin, vitamin A2 (3,4-didehydroretinol), α-vitamin A (4,5-didehydro-5,6-dihydroretinol), 13-cis-retinol, 13-cis retinoic acid (isotretinoin), 9-cis retinoic acid (9-cis-tretinoin), 4-hydroxy all-trans retinoic acid, torularodin, methyl retinoate, retinaldehyde, 13-cis-retinal, etretinate, tazoretene, acetretin, alitretinoin and adapelene.

In certain embodiments, the composition comprises a prostaglandin and a retinoid wherein the prostaglandin is alprostidil, misoprostil, dinoprostone, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, ornoprostil, 13,14-dihydroprostaglandin F2α or prostaglandin J. In certain specific embodiments, the prostaglandin is alprostadil or misoprostil. In certain embodiments, the retinoid is retinoid is tretinoin, retinal, retinol, vitamin A2, α-vitamin A, 13-cis-retinol, isotretinoin, 9-cis-tretinoin, 4-hydroxy all-trans retinoic acid, torularodin, methyl retinoate, retinaldehyde, 13-cis-retinal, etretinate, tazoretene, acetretin, alitretinoin or adapelene. In a specific embodiment, the retinoid is tretinoin or retinol. In one specific embodiment, the prostaglandin is alprostidil and the retinoid is tretinoin or retinol.

Drug Combination Comprising an Azole and a Steroid

In another embodiment, the drug combination that has anti-scarring activity comprises at least two agents wherein at least one agent is an azole, and at least one second agent is a steroid. A combination of an azole and a steroid also is capable of substantially suppressing TNF-α levels induced in white blood cells and has anti-inflammatory activity (i.e., reduces an immune response). In one embodiment, the azole is an imidazole or a triazole and the steroid is a corticosteroid, such as a glucocorticoid or a mineralocorticoid.

The azole/steroid combinations result in the unexpected enhancement of the steroid activity by as much as 10-fold when steroid is combined with a subtherapeutic dose of an azole, even when the azole is administered at a dose lower than that known to be effective as an anti-fungal agent. For example, ketoconazole is often administered at 200 mg/day orally and reaches a serum concentration of about 3.2 micrograms, while prednisone is generally administered in amounts between 5-200 mg. A 10-fold increase in the potency of the steroid can be achieved by combining it at 5 mg/day with 100 mg ketoconazole. The specific amounts of the azole (e.g., an imidazole or a triazole) and a steroid (e.g., a corticosteroid, such as a glucocorticoid or a mineralocorticoid) in the drug combination depend on the specific combination of components (i.e., the specific azole/steroid combination) and can be determined by one skilled in the art.

The azole may be selected from an imidazole or a triazole. In certain embodiments, the imidazole is selected from sulconazole, miconazole, clotrimazole, oxiconazole, butocontazole, tioconazole, econazole, and ketoconazole. In other certain embodiments, the triazole is selected from itraconazole, fluconazole, voriconazole, posaconazole, ravuconazole, and terconazole.

In certain embodiments, the drug combination comprises an azole selected from sulconazole, miconazole, clotrimazole, oxiconazole, butocontazole, tioconazole, econazole, and ketoconazole, or itrazonazole, fluconazole, voriconazole, posaconazole, ravuconazole, and terconazole, and a second compound is selected from dexamethasone, hydrocortisone, methylprednisolone, prednisone, traimcinolone, and diflorasone.

By “azole” is meant any member of the class of anti-fungal compounds having a five-membered ring of three carbon atoms and two nitrogen atoms (e.g., the imidazoles) or two carbon atoms and three nitrogen atoms (e.g., triazoles), which are capable of inhibiting fungal growth. A compound is considered “anti-fungal” if it inhibits growth of a species of fungus in vitro by at least 25%. Typically, azoles are administered in dosages of greater than 200 mg per day when used as an anti-fungal agent. Exemplary azoles for use in the invention are described herein.

Anti-fungal azoles (e.g., imidazoles and triazoles) as described herein refer to any member of the class of anti-fungal compounds having a five-membered ring of three carbon atoms and two nitrogen atoms (imidazoles) or two carbon atoms and three nitrogen atoms (triazoles). Exemplary azoles are described above.

As previously described herein by “corticosteroid” is meant any naturally occurring or synthetic steroid hormone that can be derived from cholesterol and is characterized by a hydrogenated cyclopentanoperhydrophenanthrene ring system. Naturally occurring corticosteriods are generally produced by the adrenal cortex. Synthetic corticosteriods may be halogenated. Functional groups required for activity include a double bond at Δ4, a C3 ketone, and a C20 ketone. Corticosteroids may have glucocorticoid and/or mineralocorticoid activity. Examples of exemplary corticosteroids are described above.

Corticosteroids are described in detail herein and refer to a class of adrenocortical hormones that include glucocorticoids, mineralocorticoids, and androgens, which are derived from cholesterol and is characterized by a hydrogenated cyclopentanoperhydrophenanthrene ring system. Exemplary corticosteroids are described herein and include, for example, budesonide and analogs of budesonide (e.g., budesonide (11-beta,16-alpha(R)), budesonide (11-beta,16-alpha(S)), flunisolide, desonide, triamcinolone acetonide, halcinonide, flurandrenolide, fluocinolone acetonide, triamcinolone hexacetonide, triamcinolone diacetate, flucinonide, triamcinolone, amcinafal, deflazacort, algestone, procinonide, flunisolide, hyrcanoside, descinolone, wortmannin, formocortal, tralonide, flumoxonide, triamcinolone acetonide 21-palmitate, and flucinolone, desonide, dexamethasone, desoximetasone, betamethasone, fluocinolide, triamcinolone, triamcinolone acetonide, triamcinolone diacetate, triamcinolone hexacetonide, beclomethasone dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, fludrocortisone, paramethasone acetate, prednisolone, prednisone, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone-2,1-acetate, betamethasone-1,7-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone-21-hemisuccinate free acid, prednisolone-21-acetate, prednisolone-21 (-beta-D-glucuronide), prednisolone metasulphobenzoate, prednisolone terbutate, 6-alpha-methylprednisolone, 6-alpha-methylprednisolone 21-hemisuccinate sodium salt, 6-alpha-fluoroprednisolone, 6-alpha-methylprednisolone 21-acetate, 6-alpha,9-alpha-difluoroprednisolone 21-acetate 17-butyrate, prednisolone metasulphobenzoate, cortodoxone, isoprednidene, 21-deoxycortisol, prednylidene, deprodone, 6-beta-hydroxycortisol, and triamcinolone acetonide-21-palmitate. In certain embodiments, the corticosteroid is selected from cortisone, dexamethasone, hydrocortisone, methylprenisolone, prednisone, traimcinolone, and diflorasone.

In certain embodiments, the corticosteroid is a glucocorticoid or a mineralocorticoid, and the azole is an imidazole, which is selected sulconazole, miconazole, clotrimazole, oxiconazole, butocontazole, tioconazole, econazole, and ketoconazole. In another embodiment, the azole is an itrazonazole and is selected from sulconazole, miconazole, clotrimazole, oxiconazole, butocontazole, tioconazole, econazole, and ketoconazole. In another embodiment, the azole is a triazole is selected from itrazonazole, fluconazole, voriconazole, posaconazole, ravuconazole, and terconazole. In one embodiment, the corticosteroid is a glucocorticoid selected from cortisone, dexamethasone, hydrocortisone, methylprednisolone, prednisone, traimcinolone, and diflorasone. In certain embodiments, the drug combination comprises an azole compound selected from sulconazole, miconazole, clotrimazole, oxiconazole, butocontazole, tioconazole, econazole, and ketoconazole, or itrazonazole, fluconazole, voriconazole, posaconazole, ravuconazole, and terconazole; and comprises a steroid selected from dexamethasone, hydrocortisone, methylprednisolone, prednisone, traimcinolone, and diflorasone. In one specific embodiment, the drug combination comprises dexamethasone and econazole, and in another specific embodiment, the drug combination comprises diflorasone and clotrimazole.

In another particular embodiment, the drug combination comprises an azole and a steroid, with the proviso that the amount of the azole present in the composition is not sufficient for the composition to be administered as an effective anti-fungal agent. In a preferred embodiment, the azole and steroid are present in amounts in which the activity of the steroid is enhanced at least 10-fold by the presence of the azole. In another certain embodiment, the ratio of azole to steroid (e.g., fluconazole to glucocorticoid) is about 50:1 by weight, more desirably at least about 20:1 or 10:1 by weight, and most desirably about 4:1, 2:1, or 1:1 by weight.

Compounds useful for drug combinations described herein include those described herein in any of their pharmaceutically acceptable forms, including isomers such as diastereomers and enantiomers, salts, solvates, and polymorphs thereof, as well as racemic mixtures of the compounds described herein.

Drug Combination Comprising a Steroid and (A) a Protaglandin; (B) a Beta-Adrenergic Receptor Ligand; (C) an Anti-Mitotic Agent; or (D) a Microtubule Inhibitor; and Other Combinations Thereof

In one embodiment, a drug combination that has anti-scarring activity comprises at least two agents wherein at least one agent is a steroid and at least one second agent is selected from a prostaglandin, a beta-adrenergic receptor ligand, an anti-mitotic agent, and a microtubule inhibitor. In other embodiments, the drug combination comprises an anti-mitotic agent, such as an azole, and a microtubule inhibitor.

In particular embodiments, a drug combination comprises a steroid and a prostaglandin wherein the prostaglandin is alprostadil and the steroid is diflorasone, prednisolone, or dexamethasone. In another embodiment, the drug combination comprises a beta-adrenergic receptor ligand and a steroid. In still another embodiment, an anti-mitotic agent such as podofilox (podophyllotoxin) is combined with a steroid (such as diflorasone, prednisolone, or dexamethasone)

In certain embodiments, the drug combination comprises a microtubule inhibitor (e.g., colchicine and vinblastine) and a steroid such as diflorasone, prednisolone, or dexamethasone. In yet another embodiment a microtubule inhibitor (e.g., colchicine and a vinca alkaloid (e.g., vinblastine)) is combined with an anti-mitotic agent that is an azole (e.g., clotrimazole). For example vinblastine can be used in combination with clotrimazole. Additional drug combinations comprise one or more of the compounds described above (i.e., a prostaglandin, a beta-adrenergic receptor ligand, an anti-mitotic agent, or a microtubule inhibitor in combination with a steroid, and a microtubule inhibitor in combination with an azole) include in particular embodiments, for example, a prostaglandin that is alprostidil and a steroid that is diflorasone; a beta-adrenergic receptor ligand that is isoproterenol and a steroid that is prednisolone; an anti-mitotic agent that is podofilox and a steroid that is dexamethasone; a microtubule inhibitor that is colchicine and a steroid that is flumethasone; and a microtubule inhibitor that is vinblastine and an anti-mitotic agent that is the azole, clotrimazole.

A drug combination comprising at least one steroid and at least one of a prostaglandin, beta-adrenergic receptor ligand, anti-mitotic agent or microtubule inhibitor has the capability to substantially suppress TNFα levels induced in white blood cells. TNFα is a major mediator of inflammation. Specific blockade of TNFα by using antibodies that specifically bind to TNFα or by using soluble receptors is a potent treatment for patients having an inflammatory disease. Moreover, based on the shared action among prostaglandin family members, among beta-adrenergic receptor ligand family members, among anti-mitotic agent family members, among microtubule inhibitor family members, and among steroid family members, any member of each family can be replaced by another member of that family in the combination.

In addition, the combination of a microtubule inhibitor with an azole also provides substantial suppression of TNFα levels induced in white blood cells. Thus, this drug combination can similarly be used to reduce an immune response, such as inhibit or reduce an inflammatory response (or inflammation). Based on the shared action among microtubule inhibitor family members and azole family members, one member of a family can be replaced by another member of that family in the combination.

In certain embodiments, the drug combination has certain dose combinations, for example, the ratio of prostaglandin (e.g., alprostadil) to steroid (e.g., diflorasone) may be 10:1 to 20:1 by weight; the ratio of beta-adrenergic receptor ligand (e.g., isoproterenol) to steroid (e.g., prednisolone, glucocorticoid, mineralocorticoid) may be 10:1 to 100:1 by weight; the ratio of anti-mitotic agent (e.g., podofilox) to steroid (e.g., dexamethasone) may be 10:1 to 500:1 by weight; the ratio of microtubule inhibitor (e.g., colchicine) to steroid (e.g., flumethasone) may be 50:1 to 1000:1 by weight; and the ratio of microtubule inhibitor (e.g., vinblastine) to azole (e.g., clotrimazole) may be 2:1 to 1:2 by weight.

Compounds useful in the drug combinations described herein include those described herein in any of their pharmaceutically acceptable forms, including isomers such as diastereomers and enantiomers, salts, solvates, and polymorphs thereof, as well as racemic mixtures of the compounds described herein.

By “anti-mitotic agent” is meant an agent that is capable of inhibiting mitosis. Exemplary anti-mitotic agents include, for example, podofilox, etoposide, teniposide, and griseofulvin.

By “azole” is meant any member of the class of anti-fungal compounds having a five-membered ring of three carbon atoms and two nitrogen atoms (e.g., the imidazoles) or two carbon atoms and three nitrogen atoms (e.g., triazoles), which are capable of inhibiting fungal growth. A compound is considered “anti-fungal” if it inhibits growth of a species of fungus in vitro by at least 25%. Typically, azoles are administered in dosages of greater than 200 mg per day when used as an anti-fungal agent. The azole can be selected from an imidazole or a triazole. Examples of exemplary imidazoles include but are not limited to sulconazole, miconazole, clotrimazole, oxiconazole, butocontazole, tioconazole, econazole, and ketoconazole. Examples of exemplary triazoles include but are not limited to itraconazole, fluconazole, voriconazole, posaconazole, ravuconazole, and terconazole.

By “beta-adrenergic receptor ligand” is meant an agent that binds the beta-adrenergic receptor in a sequence-specific manner. Exemplary beta-adrenergic receptor ligands include agonists and antagonists. Exemplary beta-adrenergic receptor agonists include, for example, isoproterenol, dobutamine, metaproterenol, terbutaline, isoetharine, finoterol, formoterol, procaterol, ritodrine, salmeterol, bitolterol, pirbuterol, albuterol, levalbuterol, epinephrine, and ephedrine. Exemplary beta-adrenergic receptor antagonists include, for example, propanolol, nadolol, timolol, pindolol, labetolol, metoprolol, atenolol, esmolol, acebutolol, carvedilol, bopindolol, carteolol, oxprenolol, penbutolol, medroxalol, bucindolol, levobutolol, metipranolol, bisoprolol, nebivolol, betaxolol, celiprolol, solralol, and propafenone.

By “microtubule inhibitor” is meant an agent that is capable of affecting the equilibrium between free tubulin dimers and assembled polymers. Exemplary microtubule inhibitors include, for example, colchicine, vinca alkaloids (e.g., vinblastine, vincristine, vinorelbine, and vindesine), paclitaxel, and docetaxel.

By “prostaglandin” is meant a member of the lipid class of biochemicals that belongs to a subclass of lipids known as the eicosanoids, because of their structural similarities to the C-20 polyunsaturated fatty acids, the eicosaenoic acids. Exemplary prostaglandins include alprostidil, dinoprostone, misoprostil, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, ornoprostil, 13,14-dihydroprostaglandin F2α, and prostaglandin J.

By “steroid” is meant any naturally occurring or synthetic hormone that can be derived from cholesterol and is characterized by a hydrogenated cyclopentanoperhydrophenanthrene ring system. Naturally occurring steroids are generally produced by the adrenal cortex. Synthetic steroids may be halogenated. Steroids may have corticoid, glucocorticoid, and/or mineralocorticoid activity. Examples of steroids are algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-1,7-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-2,1-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin, and other corticosteroids and steroids described herein. Desirably, the corticosteroid is selected from cortisone, dexamethasone, hydrocortisone, methylprednisolone, prednisone, traimcinolone, and diflorasone.

Accordingly in certain embodiments, a drug combination comprises a prostaglandin and a steroid, and in certain particular embodiments, the prostaglandin is alprostidil, misoprostil, dinoprostone, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, omoprostil, 13,14-dihydroprostaglandin F2α, or prostaglandin J. In a particular embodiment, the prostaglandin is alprostidil. In a more specific embodiment, the prostaglandin is alprostidil and the steroid is diflorasone.

In another embodiment, the composition comprises beta-adrenergic receptor ligand and a steroid, and in particular embodiments, the beta-adrenergic receptor ligand is isoproterenol, dobutamine, metaproterenol, terbutaline, isoetharine, finoterol, formoterol, procaterol, ritodrine, salmeterol, bitolterol, pirbuterol, albuterol, levalbuterol, epinephrine, ephedrine, propanolol, nadolol, timolol, pindolol, labetolol, metoprolol, atenolol, esmolol, acebutolol, carvedilol, bopindolol, carteolol, oxprenolol, penbutolol, medroxalol, bucindolol, levobutolol, metipranolol, bisoprolol, nebivolol, betaxolol, celiprolol, solralol, or propafenone. In a certain specific embodiment, the beta-adrenergic receptor ligand is isoproterenol. In another specific embodiment, the beta-adrenergic receptor ligand is isoproterenol and the steroid is prednisolone.

In still another embodiment, a composition comprises anti-mitotic agent and a steroid, wherein in certain embodiments, the anti-mitotic agent is podofilox, etoposide, teniposide, or griseofulvin. In a more specific embodiment, the antimitotic agent is podofilox. In another specific embodiment, the anti-mitotic agent is podofilox and the steroid is dexamethasone.

In other embodiment, the composition comprises a microtubule inhibitor and a steroid, and in specific embodiments, the microtubule inhibitor is an alkaloid, paclitaxel, or docetaxel, and wherein the alkaloid is colchicine or a vinca alkaloid. In certain embodiments, the vinca alkaloid is vinblastine, vincristine, vinorelbine, or vindesine. In other certain embodiments, the microtubule inhibitor is colchicine and said steroid is dexamethasone. In another specific embodiment, the microtubule inhibitor is colchicine and the steroid is flumethasone.

According to all the above embodiments, the steroid may be selected from dexamethasone, diflorasone, flumethasone, or prednisolone.

In another embodiment, the drug compound comprises a microtubule inhibitor and an azole, and in particular embodiments, the microtubule inhibitor is vinblastine, vincristine, vinorelbine, or vindesine. In another particular embodiment, the microtubule inhibitor is vinblastine. In another specific embodiment, the microtubule inhibitor is vinblastine and said azole is clotrimazole. In one embodiment, the azole is an imidazole or a triazole. In specific embodiments, the imidazole is selected from suconazole, miconazole, clotrimazole, oxiconazole, butoconazole, tioconazole, econazole, and ketoconazole. In another specific embodiment, the imidazole is clotrimazole. In a specific embodiment, the triazole is selected from itraconazole, fluconazole, voriconazole, posaconazole, ravuconazole, and terconazole. In one specific embodiment, the microtubule inhibitor is vinblastine and the azole is clotrimazole

For the drug combinations that comprise a steroid, the steroid is selected from algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-1,7-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, or wortmannin.

Drug Combination Comprising a Corticosteroid and (A) Serotonin Norepinephrine Reuptake Inhibitor or (B) a Noradrenaline Reuptake Inhibitor

In one embodiment, a drug combination that has anti-scarring activity comprises at least two agents wherein at least one agent is a corticosteroid and at least one second agent is selected from a serotonin norepinephrine reuptake inhibitor (SNRI) and a noradrenaline reuptake inhibitor (NARI) (or an analog or metabolite thereof). The drug combination may further include one or more additional compounds (e.g., a glucocorticoid receptor modulator, NSAID, COX-2 inhibitor, small molecule immunomodulator, DMARD, biologic, xanthine, anticholinergic compound, beta receptor agonist, bronchodilator, non-steroidal calcineurin inhibitor, vitamin D analog, psoralen, retinoid, or 5-amino salicylic acid). In a particular embodiment, the drug combination comprises a SNRI or a NARI (or an analog or metabolite thereof) and a glucocorticoid receptor modulator. In another embodiment, a drug combination is provided that includes an SNRI or NARI (or an analog or metabolite thereof) and a second compound selected from a xanthine, anticholinergic compound, beta receptor agonist, bronchodilator, non-steroidal calcineurin inhibitor, vitamin D analog, psoralen, retinoid, and 5-amino salicylic acid.

SNRIs that can be used in the drug combinations described herein include, without limitation, duloxetine, milnacipran, nefazodone, sibutramine, and venlafaxine. NARIs that can be included in the drug combinations described herein include, without limitation, atomoxetine, reboxetine, and MCI-225.

The corticosteroid and an SNRI or an NARI contained in the drug combination may be present in amounts that together are sufficient to treat or prevent an inflammatory response, disease, or disorder in a patient or subject in need thereof.

Compounds useful in the drug combinations described herein include those described herein in any of their pharmaceutically acceptable forms, including isomers such as diastereomers and enantiomers, salts, esters, solvates, and polymorphs thereof, as well as racemic mixtures and pure isomers of the compounds described herein.

By “NARI” is meant any member of the class of compounds that (i) inhibit the uptake of norepinephrine by neurons of the central nervous system, (ii) have an inhibition constant (Ki) of 100 nM or less, and (iii) a ratio of Ki(norepinephrine) over Ki(serotonin)) of less than 0.01.

Corticosteroids and exemplary corticosteroid compounds are described in detail herein. By “corticosteroid” is meant any naturally occurring or synthetic compound characterized by a hydrogenated cyclopentanoperhydrophenanthrene ring system and having immunosuppressive and/or antinflammatory activity. Naturally occurring corticosteriods are generally produced by the adrenal cortex. Synthetic corticosteriods may be halogenated.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin, which are described in detail herein. NsIDIs also include rapamycin (sirolimus) and everolimus, which bind to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Exemplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharmaceuticals).

Serotonin Norepinephrine Reuptake Inhibitors

By “SNRI” is meant any member of the class of compounds that (i) inhibit the uptake of serotonin and norepinephrine by neurons of the central nervous system, (ii) have at least one inhibition constant (Ki) of 10 nM or less, and (iii) a ratio of Ki(norepinephrine) over Ki(serotonin)) of between 0.01 and 100, desirably between 0.1 and 10.

As described herein, a drug combination may comprise an SNRI, or a structural or functional analog thereof. Suitable SNRIs include duloxetine (Cymbalta™), milnacipran (Ixel™, Toledomin™), nefazodone (Serzone™), sibutramine (Meridia™, Reductil™), and venlafaxine (Effexor™, Efexor™, Trevilor™, Vandral™).

Duloxetine

Duloxetine has the following structure:

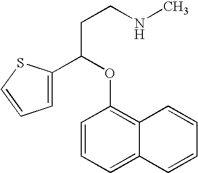

Structural analogs of duloxetine are those having the formula: 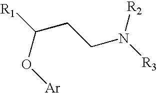
as well as pharmaceutically acceptable salts thereof, wherein R₁ is C₅-C₇ cycloalkyl, thienyl, halothienyl, (C₁-C₄alkyl) thienyl, furanyl, pyridyl, or thiazolyl; each of R₂ and R₃ Ar is, independently, hydrogen or methyl; Ar is 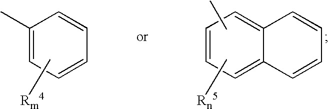
each R⁴ is, independently, halo, C₁-C₄ alkyl, C₁-C₃ alkoxy, or trifluoromethyl; each R⁵ is, independently, halo, C₁-C₄ alkyl, or trifluoromethyl; m is 0, 1, or 2; and n is 0 or 1.

Exemplary duloxetine structural analogs are N-methyl-3-(1-naphthalenyloxy)-3-(3-thienyl)propanamine phosphate; N-methyl-3-(2-naphthalenyloxy)-3-(cyclohexyl)propanamine citrate; N,N-dimethyl-3-(4-chloro-1-naphthalenyloxy)-3-(3-furanyl)propanamine hydrochloride; N-methyl-3-(5-methyl-2-naphthalenyloxy)-3-(2-thiazolyl)propanamine hydrobromide; N-methyl-3-[3-(trifluoromethyl)-1-naphthalenyloxy]-3-(3-methyl-2-thienyl)propanamine oxalate; N-methyl-3-(6-iodo-1-naphthalenyloxy)-3-(4pyridyl)propanamine maleate; N,N-dimethyl-3-(1-naphthalenyloxy)-3-(cycloheptyl)propanamine formate; N,N-dimethyl-3-(2-naphthalenyloxy)-3-(2-pyridyl)propanamine; N-methyl-3-(1-naphthalenyloxy)-3-(2-furanyl)propanamine sulfate; N-methyl-3-(4-methyl-1-naphthalenyloxy)-3-(4-thiazolyl)propanamine oxalate; N-methyl-3-(2-naphthalenyloxy)-3-(2-thienyl)propanamine hydrochloride; N,N-dimethyl-3-(6-iodo-2-naphthalenyloxy)-3-(4-bromo-3-thienyl)propanamine malonate; N,N-dimethyl-3-(1-naphthalenyloxy)-3-(3-pyridyl)propanamine hydroiodide; N,N-dimethyl-3-(4-methyl-2-naphthalenyloxy)-3-(3-furanyl)propanamine maleate; N-methyl-3-(2-naphthalenyloxy)-3-(cyclohexyl)propanamine caprate; N-methyl-3-(6-n-propyl-1-naphthalenyloxy)-3-(3-isopropyl-2-thienyl)propanamine citrate; N,N-dimethyl-3-(2-methyl-1-naphthalenyloxy)-3-(4-thiazolyl)propanamine monohydrogen phosphate; 3-(1-naphthalenyloxy)-3-(5-ethyl-3-thienyl)propanamine succinate; 3-[3-(trifluoromethyl)-1-naphthalenyloxy]-3-(pyridyl)propanamine acetate; N-methyl-3-(6-methyl-1-naphthalenyl-3-(4-chloro-2-thienyl)propanamine tartrate; 3-(2-naphthalenyloxy)-3-(cyclopentyl)propanamine; N-methyl-3-(4-n-butyl-1-naphthalenyloxy)-3-(3-furanyl)propanamine methanesulfonate; 3-(2-chloro-1-naphthalenyloxy)-3-(5-thiazolyl)propanamine oxalate; N-methyl-3-(1-naphthalenyloxy)-3-(3-furanyl)propanamine tartrate; N,N-dimethyl-3-(phenoxy)-3-(2-furanyl)propanamine oxalate; N,N-dimethyl-3-[4-(trifluoromethyl)phenoxy]-3-(cyclohexyl)propanamine hydrochloride; N-methyl-3-(4-methylphenoxy)-3-(4-chloro-2-thienyl)propanamine propionate; N-methyl-3-(phenoxy)-3-(3-pyridyl)propanamine oxalate; 3-2-chloro-4-(trifluoromethyl)phenoxy]-3-(2-thienyl)propanamine; N,N-dimethyl-3-(3-methoxyphenoxy)-3-(3-bromo-2-thienyl)propanamine citrate; N-methyl-3-(4-bromophenoxy)-3-(4-thiazolyl)propanamine maleate; N,N-dimethyl-3-(2-ethylphenoxy)-3-(5-methyl-3-thienyl)propanamine; N-methyl-3-(2-bromophenoxy)-3-(3-thienyl)propanamine succinate; N-methyl-3-(2,6-dimethylphenoxy)-3-(3-methyl-2-thienyl)propanamine acetate; 3-[3-(trifluoromethyl)phenoxy]-3-(3-furanyl)propanamine oxalate; N-methyl-3-(2,5-dichlorophenoxy)-3-(cyclopentyl)propanamine; 3-[4-(trifluoromethyl)phenoxy]-3-(2-thiazolyl)propanamine; N-methyl-3-(phenoxy)-3-(5-methyl-2-thienyl)propanamine citrate; 3-(4-methylphenoxy)-3-(4-pyridyl)propanamine hydrochloride; N,N-dimethyl-3-(3-methyl-5-bromophenoxy)-3-(3-thienyl)propanamine; N-methyl-3-(3-n-propylphenoxy)-3-(2-thienyl)propanamine hydrochloride; N-methyl-3-(phenoxy)-3-(3-thienyl)propanamine phosphate; N-methyl-3-(4-methoxyphenoxy)-3-(cycloheptyl)propanamine citrate; 3-(2-chlorophenoxy)-3-(5-thiazolyl)propanamine propionate; 3-2-chloro-4-(trifluoromethyl)phenoxy]-3-(3-thienyl)propanamine oxalate; 3-(phenoxy)-3-(4-methyl-2-thienyl)propanamine; N,N-dimethyl-3-(4-ethylphenoxy)-3-(3-pyridyl)propanamine maleate; and N,N-dimethyl-3-[4-(trifluoromethyl)phenoxy]-3-(2-pyridyl)propanamine. These compounds can be synthesized, for example, using the methods described in U.S. Pat. No. 4,956,388.

Milnacipram

Milnacipram has the following structure:

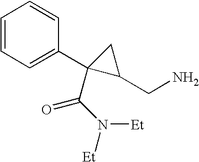

Structural analogs of milnacipram are those having the formula: 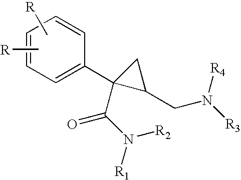
as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Nefazodone

Nefazodone has the following structure:

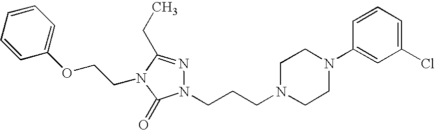

Structural analogs of nefazodone are those compounds having the formula: 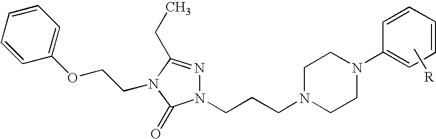
as well as pharmaceutically acceptable salts thereof, wherein R is halogen. Compounds having this formula can be synthesized, for example, using the methods described in U.S. Pat. No. 4,338,317.

Sibutramine

Sibutramine has the following structure:

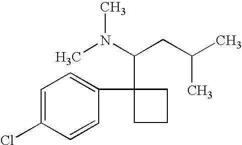

Structural analogs of sibutramine are those compounds having the formula: 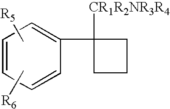
as well as pharmaceutically acceptable salts thereof, wherein R₁ is C₁₋₆ alkyl, C₂₋₆ alkenyl, C₂₋₆ alkynyl, C₃₋₇ cycloalkyl, cycloalkylalkyl, or optionally substituted phenyl (substitutents include halogen and C₁₋₃ alkyl); R₂ is H or C₁₋₃ alkyl; each of R₃ and R₄ is, independently, H, formyl, or R₃ and R₄ together with the nitrogen atom form a heterocyclic ring system; each of R₅ and R₆ is, independently, H, halogen, CF₃, C₁₋₃ alkyl, C₁₋₃ alkoxy, C₁₋₃ alkylthio, or R₆ together with the carbon atoms to which they are attached form a second benzene ring.

Exemplary sibutramine structural analogs are 1-[1-(3,4-dichlorophenyl)cyclobutyl]ethylamine hydrochloride; N-methyl-1-[1-(3,4-dichlorophenyl)cyclobutyl]ethylamine hydrochloride; N,N-dimethyl-1-[1-(3,4-dichlorophenyl)cyclobutyl]ethylamine hydrochloride; 1-[1-(4-iodophenyl)cyclobutyl]ethylamine hydrochloride; N-methyl-1-[1-(4-iodophenyl)cyclobutyl]ethylamine hydrochloride; N,N-dimethyl-1-[1-(4-iodophenyl)cyclobutyl]ethylamine hydrochloride; N-methyl-1-[1-(2-naphthyl)cyclobutyl]ethylamine hydrochloride; N,N-dimethyl-1-[1-(4-chloro-3-trifluoromethylphenyl)cyclobutyl]ethylamine hydrochloride; 1-[1-(4-chlorophenyl)cyclobutyl]butylamine hydrochloride; N-methyl-1-[1-(4-chlorophenyl)cyclobutyl]butylamine hydrochloride; N,N-dimethyl-1-[1-(4-chlorophenyl)cyclobutyl]butyl amine hydrochloride; 1-[1-(3,4-dichlorophenyl)cyclobutyl]butylamine hydrochloride; N-methyl-1-[1-(3,4-dichlorophenyl)cyclobutyl]butylamine hydrochloride; N,N-dimethyl-1-[1-(3,4-dichlorophenyl)cyclobutyl]butylamine hydrochloride; 1-[1-(4-biphenylyl)cyclobutyl]butylamine hydrochloride; N,N-dimethyl-1-[1-(4-biphenylyl)cyclobutyl]butylamine hydrochloride; 1-[1-(4-chloro-3-fluorophenyl)cyclobutyl]butylamine hydrochloride; N-formyl-1-[1-(4-chloro-3-fluorophenyl)cyclobutyl]butylamine; 1-[1-(3-chloro-4-methylphenyl)cyclobutyl]butylamine hydrochloride; N-formyl-1-[1-phenylcyclobutyl]butylamine; 1-[1-(3-trifluoromethylphenyl)cyclobutyl]butylamine hydrochloride; 1-[1-(naphth-2-yl)cyclobutyl]butylamine hydrochloride; 1-[1-(6-chloronaphth-2-yl)cyclobutyl]butylamine; N-methyl-1-[1-(4-chlorophenyl)cyclobutyl]-2-methylpropylamine hydrochloride; 1-[1-(4-chlorophenyl)cyclobutyl]pentyl amine hydrochloride; N-methyl-1-[1-(4-chlorophenyl)cyclobutyl]pentylamine hydrochloride; N,N-dimethyl-1-[1-phenylcyclobutyl]-3-methylbutylamine hydrochloride; 1-[1-(4-chlorophenyl)cyclobutyl]-3-methylbutylamine hydrochloride; N-methyl-1-[1-(4-chlorophenyl)cyclobutyl]-3-methylbutylamine hydrochloride; N,N-dimethyl-1-[1-(4-chlorophenyl)cyclobutyl]-3-methylbutylamine hydrochloride; N-formyl-1-[1-(4-chlorophenyl)cyclobutyl]-3-methylbutylamine; N,N-dimethyl-1-[1-(3,4-dichlorophenyl)cyclobutyl]-3-methylbutylamine hydrochloride; N-methyl-1-[1-(naphth-2-yl)cyclobutyl]-3-methylbutylamine hydrochloride; N-methyl-1-[1-(3,4-dimethylphenyl)cyclobutyl]-3-methylbutylamine hydrochloride; [1-(4-chlorophenyl)cyclobutyl](cyclopropyl)methylamine hydrochloride; N-methyl-[1-(4-chlorophenyl)cyclobutyl](cyclopentyl)methylamine hydrochloride; [1-(4-chlorophenyl)cyclobutyl](cyclohexyl)methylamine hydrochloride; N-methyl-[1-(4-chlorophenyl)cyclobutyl](cyclohexyl)methylamine hydrochloride; [1-(3,4-dichlorophenyl)cyclobutyl](cyclohexyl)methylamine hydrochloride; N-methyl-[1-(3,4-dichlorophenyl)cyclobutyl](cyclohexyl)methylamine hydrochloride; [1-(4-chlorophenyl)cyclobutyl](cycloheptyl)methylamine hydrochloride; 1-[1-(4-chlorophenyl)cyclobutyl]-2-cyclopropylethylamine hydrochloride; N,N-dimethyl-1-[1-(4-chlorophenyl)cyclobutyl]-2-cyclohexylethylamine hydrochloride; α-[1-(4-chlorophenyl)cyclobutyl]benzylamine hydrochloride; N-methyl-α-[1-(4-chlorophenyl)cyclobutyl]benzylamine hydrochloride; 1-[1-(4-chloro-2-fluorophenyl)cyclobutyl]butylamine; N,N-dimethyl-1-[1-(4-chloro-2-fluorophenyl)cyclobutyl]butylamine hydrochloride; N-ethyl-1-[1-(3,4-dichlorophenyl)cyclobutyl]ethylamine hydrochloride; and N,N-diethyl-1-[1-(3,4-dichlorophenyl)cyclobutyl]ethylamine hydrochloride. These compounds can be synthesized, for example, using the methods described in U.S. Pat. No. 4,814,352.

Venlafaxine

Venlafaxine has the following structure:

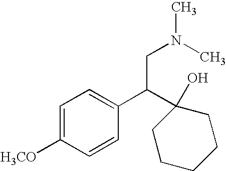

Structural analogs of venlafaxine are those compounds having the formula: 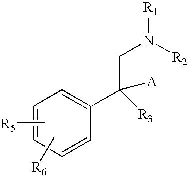
as well as pharmaceutically acceptable salts thereof, wherein A is a moiety of the formula: 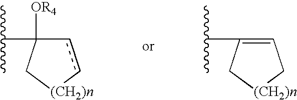
where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.
Noradrenaline Reuptake Inhibitors

The drug combinations described herein may comprise an NARI, or a structural or functional analog thereof. Suitable NARI compounds include atomoxetine (Strattera™), reboxetine (Edronax™), and MCI-225.

Atomoxetine

Atomoxetine has the following structure:

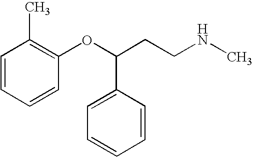

Structural analogs of atomoxetine are those having the formula: 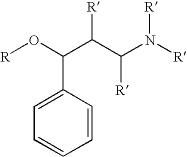
as well as pharmaceutically acceptable salts thereof, wherein each R′ is, independently, hydrogen or methyl; and R is napthyl or 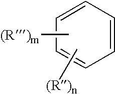
wherein each of R″ and R′″ is, independently, halo, trifluoromethyl, C₁₋₄ alkyl, C₁₋₃ alkoxy, or C₃₋₄ alkenyl; and each of n and m is, independently, 0, 1, or 2.

Exemplary atomoxetine structural analogs are 3-(p-isopropoxyphenoxy)-3-phenylpropylamine methanesulfonate; N,N-dimethyl 3-(3′,4′-dimethoxyphenoxy)-3-phenylpropylamine p-hydroxybenzoate; N,N-dimethyl 3-(α-naphthoxy)-3-phenylpropylamine bromide; N,N-dimethyl 3-(.beta.-naphthoxy)-3-phenyl-1-methylpropylamine iodide; 3-(2′-methyl-4′,5′-dichlorophenoxy)-3-phenylpropylamine nitrate; 3-(p-t-butylphenoxy)-3-phenylpropyl amine glutarate; N-methyl 3-(2′-chloro-p-tolyloxy)-3-phenyl-1-methylpropylamine lactate; 3-(2′,4′-dichlorophenoxy)-3-phenyl-2-methylpropylamine citrate; N,N-dimethyl 3-(m-anisyloxy)-3-phenyl-1-methylpropylamine maleate; N-methyl 3-(p-tolyloxy)-3-phenylpropylamine sulfate; N,N-dimethyl 3-(2′,4′-difluorophenoxy)-3-phenylpropylamine 2,4-dinitrobenzoate; 3-(o-ethylphenoxy)-3-phenylpropylamine dihydrogen phosphate; N-methyl 3-(2′-chloro-4′-isopropylphenoxy)-3-phenyl-2-methylpropylamine maleate; N,N-dimethyl 3-(2′-alkyl-4′-fluorophenoxy)-3-phenyl-propylamine succinate; N,N-dimethyl 3-(o-isopropoxyphenoxy)-3-phenyl-propylamine phenylacetate; N,N-dimethyl 3-(o-bromophenoxy)-3-phenyl-propylamine .beta.-phenylpropionate; N-methyl 3-(p-iodophenoxy)-3-phenyl-propylamine propiolate; and N-methyl 3-(3-n-propylphenoxy)-3-phenyl-propylamine decanoate. These compounds can be synthesized, for example, using the methods described in U.S. Pat. No. 4,314,081.

Reboxetine

Reboxetine has the following structure:

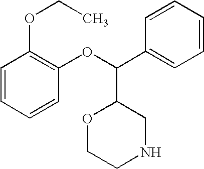

Structural analogs of reboxetine are those having the formula: 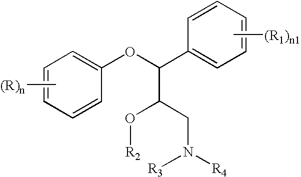
as well as pharmaceutically acceptable salts thereof, wherein each of n and nil is, independently, 1, 2, or 3; each of R and R₁ is, independently, hydrogen, halogen, halo-C₁₋₆ alkyl, hydroxy, C₁₋₆ alkyl optionally substituted, C₁₋₆ alkoxy, aryl-C₁₋₆ alkoxy optionally substituted, NO₂, NR₅R₆, wherein each of R₅ and R₆ is, independently, hydrogen, C₁₋₆ alkyl, or two adjacent R groups or two adjacent R₁ groups, taken together, form the —O—CH₂—O— radical; R₂ is hydrogen; C₁₋₁₂ alkyl optionally substituted, or aryl-C₁₋₆ alkyl; each of R₃ and R₄ is, independently, hydrogen, C₁₋₆ alkyl optionally substituted, C₂₋₄ alkenyl, C₂₋₄ alkynyl, aryl-C₁₋₄ alkyl optionally substituted, C₃₋₇ cycloalkyl optionally substituted, or R₃ and R₄ with the nitrogen atom to which they are bounded form a pentatomic or hexatomic saturated or unsaturated, optionally substituted, heteromonocyclic radical optionally containing other heteroatoms belonging to the class of O, S and N; or R₂ and R₄, taken together, form the —CH₂CH₂— radical.

Exemplary reboxetine structural analogs are 2-(α-phenoxy-benzyl)-morpholine; 2-[α-(2-methoxy-phenoxy)-benzyl]-morpholine; 2-[α-(3-methoxy-phenoxy)-benzyl]-morpholine; 2-[α-(4-methoxy-phenoxy)-benzyl]-morpholine; 2-[α-(2-ethoxy-phenoxy)-benzyl]-morpholine; 2-[α-(4-chloro-phenoxy)-benzyl]-morpholine; 2-[α-(3,4-methylendioxy-phenoxy)-benzyl]-morpholine; 2-[α-(2-methoxy-phenoxy)-2-methoxy-benzyl]-morpholine; 2-[α-(2-ethoxy-phenoxy)-2-methoxy-benzyl]-morpholine; 2-[α-(2-ethoxy-phenoxy)-4-ethoxy-benzyl]-morpholine; 2-[α-(4-chloro-phenoxy)-4-ethoxy-benzyl]-morpholine; 2-[α-(2-methoxy-phenoxy)-4-ethoxy-benzyl]-morpholine; 2-[α-(2-methoxy-phenoxy)-2-chloro-benzyl]-morpholine; 2-[α-(2-ethoxy-phenoxy)-2-chloro-benzyl]-morpholine; 2-[α-(2-methoxy-phenoxy)-3-chloro-benzyl]-morpholine; 2-[α-(2-ethoxy-phenoxy)-3-chloro-benzyl]-morpholine; 2-[α-(2-ethoxy-phenoxy)-4-chloro-benzyl]-morpholine; 2-[α-(2-methoxy-phenoxy)-4-chloro-benzyl]-morpholine; 2-[α-(2-methoxy-phenoxy)-4-trifluoromethyl-benzyl]-morpholine; 2-[α-(4-ethoxy-phenoxy)-4-trifluoromethyl-benzyl]-morpholine; 2-[α-(2-methoxy-phenoxy)-3,4-dichloro-benzyl]-morpholine; 2-[α-(2-ethoxy-phenoxy)-3,4-dichloro-benzyl]-morpholine; 4-methyl-2-[α-(2-methoxy-phenoxy)-benzyl]-morpholine; 4-methyl-2-[α-(2-ethoxy-phenoxy)-benzyl]-morpholine; 4-methyl-2-[α-(2-methoxy-phenoxy)-3-chloro-benzyl]-morpholine; 4-methyl-2-[α-(2-ethoxy-phenoxy)-3-chloro-benzyl]-morpholine; 4-methyl-2-[α-(2-ethoxy-phenoxy)-4-chloro-benzyl]-morpholine; 4-methyl-2-[α-(2-methoxy-phenoxy)-4-chloro-benzyl]-morpholine; 4-methyl-2-[α-(2-methoxy-phenoxy)-4-trifluoromethyl-benzyl]-morpholine; 4-methyl-2-[α-(2-ethoxy-phenoxy)-4-trifluoromethyl-benzyl]-morpholine; 4-isopropyl-2-[α-(2-methoxy-phenoxy)-benzyl]-morpholine; 4-isopropyl-2-[α-(2-ethoxy-phenoxy)-benzyl]-morpholine; 4-isopropyl-2-[α-(2-methoxy-phenoxy)-3-chloro-benzyl]-morpholine; 4-isopropyl-2-[α-(2-ethoxy-phenoxy)-3-chloro-benzyl]-morpholine; 4-isopropyl-2-[α-(2-ethoxy-phenoxy)-4-chloro-benzyl]-morpholine; 4-isopropyl-2-[α-(2-methoxy-phenoxy)-4-chloro-benzyl]-morpholine; 4-isopropyl-2-[α-(2-methoxy-phenoxy)-4-trifluoromethyl-benzyl]-morpholine; 4-isopropyl-2-[α-(2-ethoxy-phenoxy)-4-trifluoromethyl-benzyl]-morpholine; N-methyl-2-hydroxy-3-phenoxy-3-phenyl-propylamine; N-methyl-2-hydroxy-3-(2-methoxy-phenoxy)-3-phenyl-propylamine; N-methyl-2-hydroxy-3-(2-ethoxy-phenoxy)-3-phenyl-propylamine; N-methyl-2-hydroxy-3-(4-chloro-phenoxy)-3-phenyl-propylamine; N-methyl-2-hydroxy-3-(3,4-methylendioxy-phenoxy)-3-phenyl-propylamine; N-methyl-2-hydroxy-3-(2-methoxy-phenoxy)-3-(2-chloro-phenyl)-propylamine; N-methyl-2-hydroxy-3-(2-ethoxy-phenoxy)-3-(2-chloro-phenyl)-propylamine; N-methyl-2-hydroxy-3-(2-methoxy-phenoxy)-3-(3-chloro-phenyl)-propylamine; N-methyl-2-hydroxy-3-(2-ethoxy-phenoxy)-3-(3-chloro-phenyl)-propylamine; N-methyl-2-hydroxy-3-(2-methoxy-phenoxy)-3-(4-chloro-phenyl)-propylamine; N-methyl-2-hydroxy-3-(2-ethoxy-phenoxy)-3-(4-chloro-phenyl)-propylamine; N-methyl-2-hydroxy-3-(2-methoxy-phenoxy)-3-(4-trifluoromethyl-phenyl)-propylamine; N-methyl-2-hydroxy-3-(2-ethoxy-phenoxy)-3-(4-trifluoromethyl-phenyl)-propyl amine; N-methyl-2-hydroxy-3-(2-methoxy-phenoxy)-3-(3,4-dichloro-phenyl)-propylamine; N-methyl-2-hydroxy-3-(2-ethoxy-phenoxy)-3-(3,4-dichloro-phenyl)-propylamine; N-methyl-2-methoxy-3-phenoxy-3-phenyl-propylamine; N-methyl-2-methoxy-3-(2-methoxy-phenoxy)-3-phenyl-propylamine; N-methyl-2-methoxy-3-(2-ethoxy-phenoxy)-3-phenyl-propylamine; N-methyl-2-methoxy-3-(4-chloro-phenoxy)-3-phenyl-propylamine; N-methyl-2-methoxy-3-(3,4-methylenedioxy-phenoxy)-3-phenyl-propylamine; N-methyl-2-methoxy-3-phenoxy-3-(2-chloro-phenyl)-propylamine; N-methyl-2-methoxy-3-(2-methoxy-phenoxy)-3-(2-chloro-phenyl)-propylamine; N-methyl-2-methoxy-3-(2-ethoxy-phenoxy)-3-(2-chloro-phenyl)-propylamine; N-methyl-2-methoxy-3-(2-methoxy-phenoxy)-3-(3-chloro-phenyl)-propylamine; N-methyl-2-methoxy-3-(2-ethoxy-phenoxy)-3-(3-chloro-phenyl)-propylamine; N-methyl-2-methoxy-3-(2-methoxy-phenoxy)-3-(4-chloro-phenyl)-propylamine; N-methyl-2-methoxy-3-(2-ethoxy-phenoxy)-3-(4-chloro-phenyl)-propylamine; N-methyl-2-methoxy-3-(2-methoxy-phenoxy)-3-(4-trifluoromethyl-phenyl)-propylamine; N-methyl-2-methoxy-3-(2-ethoxy-phenoxy)-3-(4-trifluoromethyl-phenyl)-propylamine; N-methyl-2-methoxy-3-(2-methoxy-phenoxy)-3-(3,4-dichloro-phenyl)-propylamine; and N-methyl-2-methoxy-3-(2-ethoxy-phenoxy)-3-(3,4-dichloro-phenyl)-propylamine. These compounds can be synthesized, for example, using the methods described in U.S. Pat. No. 4,229,449.

MCI-225

MCI-225 (4-(2-fluorophenyl)-6-methyl-2-piperazinothieno[2,3-d]pyrimidine) has the following structure:

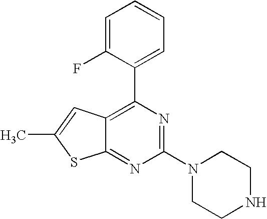

Structural analogs of MCI-225 are those having the formula: 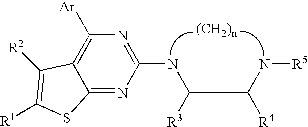
as well as pharmaceutically acceptable salts thereof, wherein each of R¹ and R² is, independently, hydrogen, halogen, C₁-C₆ alkyl, or R¹ and R² form a 5 to 6-membered cycloalkylene ring together with two carbon atoms of thienyl group; each of R³ and R⁴ is, independently, hydrogen or C₁-C₆ alkyl; R⁵ is hydrogen, C₁-C₆ alkyl, 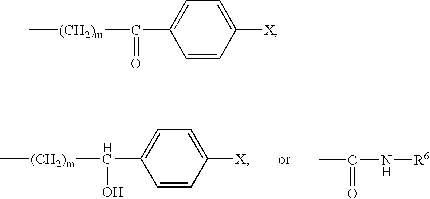
in which m is an integer of 1-3, X is a halogen, and R⁶ is C₁-C₆ alkyl; Ar is phenyl, 2-thienyl, or 3-thienyl, each of which may substituted by halogen, C₁-C₆ alkyl, C₁-C₆ alkoxy (e.g., methoxy, ethoxy, propoxy, and butoxy), hydroxyl, nitro, amino, cyano, or alkyl-substituted amino (e.g., methylamino, ethylamino, dimethylamino, and diethylamino); and n is 2 or 3.

Exemplary MCI-225 structural analogs are 6-methyl-4-phenyl-2-piperazinyl-thieno[2,3-d]pyrimidine; 5,6-dimethyl-4-phenyl-2-piperazinyl-thieno[2,3-d]pyrimidine; 5-methyl-4-phenyl-2-piperazinyl-thieno[2,3-d]pyrimidine; 6-chloro-4-phenyl-2-piperazinyl-thieno[2,3-d]pyrimidine; 4-(2-bromophenyl)-6-methyl-2-piperazinyl-thieno[2,3-d]pyrimidine; 6-methyl-4-(2-methylphenyl)-2-piperazinyl-thieno[2,3-d]pyrimidine; and 4-(2-cyanophenyl)-6-methyl-2-piperazinyl-thieno[2,3-d]. These compounds can be synthesized, for example, using the methods described in U.S. Pat. No. 4,695,568.

In still other embodiments, certain other compounds can be used in drug combinations described herein instead of an SNRI or NARI and include 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554 (piperazine, 1-(3-(1,3-benzodioxol-5-yloxy)propyl)-4-phenyl); CP 53261 (N-desmethylsertraline); O-desmethylvenlafaxine; WY 45,818 (1-(2-(dimethylamino)-1-(2-chlorophenyl)ethyl)cyclohexanol); WY 45,881 (1-(1-(3,4-dichlorophenyl)-2-(dimethylamino)ethyl)cyclohexanol); N-(3-fluoropropyl)paroxetine; and Lu 19005 (3-(3,4-dichlorophenyl)-N-methyl-1-indanamine hydrochloride).

Compounds useful for the drug combinations described herein include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “paroxetine” is meant the free base, as well as any pharmaceutically acceptable salt thereof (e.g., paroxetine maleate, paroxetine hydrochloride hemihydrate, and paroxetine mesylate).

Corticosteroids

In one embodiment, one or more corticosteroid may be combined or formulated with an SNRI or NARI, or analog or metabolite thereof, in a drug combination. Suitable corticosteroids include any one of the corticosteroid compounds described herein or known in the art.

Steroid Receptor Modulators

Steroid receptor modulators (e.g., antagonists and agonists) may be used as a substitute for or in addition to a corticosteroid in the drug combination. Thus, in one embodiment, the drug combination features the combination of an SNRI or NARI (or analog or metabolite thereof) and a glucocorticoid receptor modulator or other steroid receptor modulator.

Glucocorticoid receptor modulators that may used in the drug combinations described herein include compounds described in U.S. Pat. Nos. 6,380,207, 6,380,223, 6,448,405, 6,506,766, and 6,570,020, U.S. Patent Application Publication Nos. 20030176478, 20030171585, 20030120081, 20030073703, 2002015631, 20020147336, 20020107235, 20020103217, and 20010041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the drug combinations described herein A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

In one embodiment, as a substitute for or in addition to a corticosteroid in the drug combinations described herein, one or more agents that also act as bronchodilators may be included in the combination, including xanthines (e.g., theophylline), anticholinergic compounds (e.g., ipratropium, tiotropium), biologics, small molecule immunomodulators, and beta receptor agonists/bronchdilators (e.g., albuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, the drug combination comprises an SNRI or NARI (or analog or metabolite thereof) and/or a corticosteroid and/or one or more of the aforementioned agents.

In another embodiment, as a substitute for or in addition to a corticosteroid in the drug combinations described herein, one or more agents that also acts as antipsoriatic agents may be included in the drug combination. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the drug combination features the combination of an SNRI or NARI (or analog or metabolite thereof) and/or a corticosteroid and/or one or more of the aforementioned agents.

In another embodiment, as a substitute for or in addition to a corticosteroid in the drug combinations described herein, one or more agents typically used to treat inflammatory bowel disease may be included in the drug combination. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, the drug combinations described herein feature the combination of an SNRI or NARI (or analog or metabolite thereof) and/or a corticosteroid and/or one or more of any of the foregoing agents.

In still another embodiment, one or more agents typically used to treat rheumatoid arthritis may be used as a substitute for or in addition to a corticosteroid in the drug combinations described herein. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the drug combination features the combination of an SNRI or NARI (or analog or metabolite thereof) and/or a corticosteroid and/or one or more of any of the foregoing agents.

In yet another embodiment, one or more agents typically used to treat asthma may be used as a substitute for or in addition to a corticosteroid in the drug combinations described herein. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, a drug combination features the combination of an SNRI or NARI (or analog or metabolite thereof) and/or a corticosteroid and/or one or more of any of the foregoing agents.

Also provided herein are drug combinations employing an SNRI or NARI and a non-steroidal immunophilin-dependent immunosuppressant (NsIDI), optionally with a corticosteroid or other agent described herein.

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders.

Cyclosporines

The cyclosporines are examples of calcineurin inhibitors and are fungal metabolites that comprise a class of cyclic oligopeptides that act as immunosuppressants. As described herein, Cyclosporine A, and its deuterated analogue ISAtx247, is a hydrophobic cyclic polypeptide consisting of eleven amino acids. Cyclosporine A binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T-cell-dependent immune response by inhibiting antigen-triggered signal transduction. This inhibition decreases the expression of proinflammatory cytokines, such as IL-2.

Many cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Publication No. 20020132763). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala (3-acetylamino)-8-Cs, Thr-2-Cs, and D-MeSer-3-Cs, D-Ser (O—CH₂CH₂—OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g., on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

To counteract the hydrophobicity of cyclosporine A, an intravenous cyclosporine A is usually provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL™).

Tacrolimus

As described herein, tacrolimus (PROGRAF, Fujisawa), also known as FK506, is an immunosuppressive agent that targets T-cell intracellular signal transduction pathways. Tacrolimus binds to an intracellular protein FK506 binding protein (FKBP-12) that is not structurally related to cyclophilin (Harding et al., Nature 341:758-7601, 1989; Siekienka et al. Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of NFAT, a nuclear component that initiates gene transcription required for lymphokine (e.g., IL-2, gamma interferon) production and T-cell activation. Thus, tacrolimus inhibits T-cell activation.

Tacrolimus is a macrolide antibiotic that is produced by Streptomyces tsukubaensis. Tacrolimus suppresses the immune system and prolongs the survival of transplanted organs. Tacrolimus is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 9% sodium chloride or 5% dextrose prior to injection.

Tacrolimus and tacrolimus analogs are described by Tanaka et al., (J. Am. Chem. Soc., 109:5031, 1987), and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus and Ascomycin Derivatives

Ascomycin is a close structural analog of FK506 and is a potent immunosuppressant. It binds to FKBP-12 and suppresses its proline rotamase activity. The ascomycin-FKBP complex inhibits calcineurin, a type 2B phosphatase.

Pimecrolimus (also known as SDZ ASM-981) is a 33-epi-chloro derivative of the ascomycin. It is produced by the strain Streptomyces hygroscopicus var. ascomyceitus. Like tacrolimus, pimecrolimus (ELIDEL™, Novartis) binds FKBP-12, inhibits calcineurin phosphatase activity, and inhibits T-cell activation by blocking the transcription of early cytokines. In particular, pimecrolimus inhibits IL-2 production and the release of other proinflammatory cytokines.

Pimecrolimus structural and functional analogs are described in U.S. Pat. No. 6,384,073. Pimecrolimus is used for the treatment of atopic dermatitis. Pimecrolimus is currently available as a 1% cream.

Rapamycin

Rapamycin (Rapamune® sirolimus, Wyeth) is a cyclic lactone produced by Streptomyces hygroscopicus. Rapamycin is an immunosuppressive agent that inhibits T-lymphocyte activation and proliferation. Like cyclosporines, tacrolimus, and pimecrolimus, rapamycin forms a complex with the immunophilin FKBP-12, but the rapamycin-FKBP-12 complex does not inhibit calcineurin phosphatase activity. The rapamycin-immunophilin complex binds to and inhibits the mammalian target of rapamycin (mTOR), a kinase that is required for cell cycle progression. Inhibition of mTOR kinase activity blocks T-lymphocyte proliferation and lymphokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Everolimus (40-O-(2-hydroxyethyl)rapamycin; CERTICAN™; Novartis) is an immunosuppressive macrolide that is structurally related to rapamycin, and has been found to be particularly effective at preventing acute rejection of organ transplant when give in combination with cyclosporin A. By way of background, and as described herein, rapamycin is currently available for oral administration in liquid and tablet formulations.

Peptide Moieties

Peptides, peptide mimetics, peptide fragments, either natural, synthetic or chemically modified, that impair the calcineurin-mediated dephosphorylation and nuclear translocation of NFAT are suitable for inclusion in the drug combinations described herein. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell 1:627-637, 1998). As a class of calcinuerin inhibitors, these agents are useful in the drug combinations described herein.

In other embodiments, a drug combination may further comprise other compounds, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies may be useful for the treatment of or prevention of an inflammatory response or autoimmune response in combination with other anti-cytokine agents or in combination with agents that modulate the immune response, such as agents that influence cell adhesion, or biologics (i.e., agents that block the action of IL-6, IL-1, IL-2, IL-12, IL-15 or TNFα (e.g., etanercept, adelimumab, infliximab, or CDP-870). For example (that of agents blocking the effect of TNFα), when the combination therapy reduces the production of cytokines, etanercept or infliximab may affect the remaining fraction of inflammatory cytokines.

In certain particular embodiments, a drug combination is provided that comprises a serotonin norepinephrine reuptake inhibitor (SNRI) or noradrenaline reuptake inhibitor (NARI) or analog thereof and a corticosteroid. In a particular embodiment, the SNRI is duloxetine, milnacipran, nefazodone, sibutramine, or venlafaxine, and in another particular embodiment, the NARI is atomoxetine, reboxetine, or MCI-225. In a specific embodiment, the corticosteroid is prednisolone, cortisone, budesonide, dexamethasone, hydrocortisone, methylprednisolone, fluticasone, prednisone, triamcinolone, or diflorasone. In a more specific embodiment, the SNRI is duloxetine or venlafaxine and the corticosteroid is prednisolone. In another specific embodiment, the NARI is atomoxetine or MCI-225 and the corticosteroid is prednisolone.

In another embodiment, the drug combination may further comprise an NSAID, COX-2 inhibitor, biologic, small molecule immunomodulator, DMARD, xanthine, anticholinergic compound, beta receptor agonist, bronchodilator, non-steroidal calcineurin inhibitor, vitamin D analog, psoralen, retinoid, or 5-amino salicylic acid. In particular embodiments, the NSAID is ibuprofen, diclofenac, or naproxen, and in other particular embodiments, the COX-2 inhibitor is rofecoxib, celecoxib, valdecoxib, or lumiracoxib. In other particular embodiments, the biologic is adelimumab, etanercept, or infliximab, and in other particular embodiments, the DMARD is methotrexate or leflunomide. In one particular embodiment, the xanthine is theophylline. In another embodiment, the anticholinergic compound is ipratropium or tiotropium; in other particular embodiments, the beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline. In still other particular embodiments, the non-steroidal calcineurin inhibitor is cyclosporine, tacrolimus, pimecrolimus, or ISAtx247, and in other more particular embodiments, vitamin D analog is calcipotriene or calcipotriol. In another particular embodiment, psoralen is methoxsalen. In another embodiment, the retinoid is acitretin or tazoretene, and in another embodiment, 5-amino salicylic acid is mesalamine, sulfasalazine, balsalazide disodium, or olsalazine sodium. In an additional embodiment, a small molecule immunomodulator is VX 702, SCIO 469, doramapimod, RO 30201195, SCIO 323, DPC 333, pranalcasan, mycophenolate, or merimepodib.

Drug Combination Comprising a Non-Steroidal Immunophilin-Dependent Immunosuppressant (NsIDI) and a Non-Steroidal Immunophilin-Dependent Immunosuppressant Enhancer (NsIDIE)

In one embodiment, a drug combination that has anti-scarring activity comprises at least two agents wherein at least one agent is a non-steroidal immunophilin-dependent immunosuppressant (NsIDI) (e.g., cyclosporine A) and at least one second agent is a non-steroidal immunophilin-dependent immunosuppressant enhancer (NsIDIE) (e.g., a selective serotonin reuptake inhibitor (SSRI), a tricyclic antidepressant, a phenoxy phenol, an antihistamine, a phenothiazine, or a mu opioid receptor agonist). In certain embodiments, the drug combination may further comprise a non-steroidal anti-inflammatory drug (NSAID), a COX-2 inhibitor, a biologic, a disease-modifying anti-rheumatic drugs (DMARD), a xanthine, an anticholinergic compound, a beta receptor agonist, a bronchodilator, a non-steroidal calcineurin inhibitor, a vitamin D analog, a psoralen, a retinoid, or a 5-amino salicylic acid.

In certain embodiments described herein, an NsIDI is, for example, a calcineurin inhibitor, such as cyclosporine, tacrolimus, ascomycin, pimecrolimus, or ISAtx247, or an FK506-binding protein, such as rapamycin or everolimus. In other embodiments, an NsIDI enhancer (NsIDIE) is, for example, a selective serotonin reuptake inhibitor (SSRI), a tricyclic antidepressant (TCA), a phenoxy phenol, an antihistamine, a phenothiazine, or a mu opioid receptor agonist.

By “non-steroidal immunophilin-dependent immunosuppressant enhancer” or “NsIDIE” is meant any compound that increases the efficacy of a non-steroidal immunophilin-dependent immunosuppressant. NsIDIEs include selective serotonin reuptake inhibitors, tricyclic antidepressants, phenoxy phenols (e.g., triclosan), antihistamines, phenothiazines, and mu opioid receptor agonists.

By “antihistamine” is meant a compound that blocks the action of histamine. Classes of antihistamines include, but are not limited to, ethanolamines, ethylenediamine, phenothiazine, alkylamines, piperazines, and piperidines.

By “selective serotonin reuptake inhibitor” or “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants. Exemplary SSRIs for use in the invention are described herein.

Compounds useful for the drug combinations described herein include those described herein in any of their pharmaceutically acceptable forms, including isomers such as diastereomers and enantiomers, salts, esters, solvates, and polymorphs thereof, as well as racemic mixtures and pure isomers of the compounds described herein.

A tricyclic compound, which includes a “tricyclic antidepressant” or “TCA” compound includes a compound having one of the formulas (I), (II), (III), or (IV), which are described in greater detail herein. Exemplary tricyclic antidepressants are also provided herein and include maprotiline, amoxapine, 8-hydroxyamoxapine, 7-hydroxyamoxapine, loxapine, loxapine succinate, loxapine hydrochloride, 8-hydroxyloxapine, amitriptyline, clomipramine, doxepin, imipramine, trimipramine, desipramine, nortriptyline, and protriptyline.

By “corticosteroid” is meant any naturally occurring or synthetic compound characterized by a hydrogenated cyclopentanoperhydrophenanthrene ring system and having immunosuppressive and/or antinflammatory activity. Naturally occurring corticosteriods are generally produced by the adrenal cortex. Synthetic corticosteriods may be halogenated. Corticosteroids are described in detail herein and examples of corticosteroids are also provided herein.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Exemplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharmaceuticals).

In the generic descriptions of compounds of this invention, such as for example, with respect to the structures having any one of formula (I), (II), (III), or (IV), the number of atoms of a particular type in a substitutent group is generally given as a range, e.g., an alkyl group containing from 1 to 7 carbon atoms or C1-7 alkyl. Reference to such a range is intended to include specific references to groups having each of the integer number of atoms within the specified range. For example, an alkyl group from 1 to 7 carbon atoms includes each of C1, C2, C3, C4, C5, C6, and C7. A C1-7 heteroalkyl, for example, includes from 1 to 7 carbon atoms in addition to one or more heteroatoms. Other numbers of atoms and other types of atoms may be indicated in a similar manner.

Compounds include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “paroxetine” is meant the free base, as well as any pharmaceutically acceptable salt thereof (e.g., paroxetine maleate, paroxetine hydrochloride hemihydrate, and paroxetine mesylate).

Provided herein are drug combinations that comprise an effective amount of a non-steroidal immunophilin-dependent immunosuppressant (NsIDI), such as cyclosporine, and a non-steroidal immunophilin-dependent immunosuppressant enhancer (NSIDIE), e.g., a selective serotonin reuptake inhibitor, a tricyclic antidepressant, a phenoxy phenol, an antihistamine, a phenothiazine, or a mu opioid receptor agonist. The combinations are described in greater detail below.

Non-Steroidal Immunophilin-Dependent Immunosuppressants

In one embodiment, the drug combination comprises an NsIDI and an NsIDIE, optionally with a corticosteroid or other agent described herein. By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders. The cyclosporines, tacrolimus, ascomycin, pimecrolimus, rapamycin, and peptide moities are described in detail above.

Selective Serotonin Reuptake Inhibitors

In one embodiment, the drug combination comprises a selective serotonin reuptake inhibitor (SSRI), or a structural or functional analog thereof in combination with a non-steroidal immunophilin-dependent immunosuppressant (NsIDI). Suitable SSRIs include cericlamine (e.g., cericlamine hydrochloride); citalopram (e.g., citalopram hydrobromide); clovoxamine; cyanodothiepin; dapoxetine; escitalopram (escitalopram oxalate); femoxetine (e.g., femoxetine hydrochloride); fluoxetine (e.g., fluoxetine hydrochloride); fluvoxamine (e.g., fluvoxamine maleate); ifoxetine; indalpine (e.g., indalpine hydrochloride); indeloxazine (e.g., indeloxazine hydrochloride); litoxetine; milnacipran (e.g., minlacipran hydrochloride); paroxetine (e.g., paroxetine hydrochloride hemihydrate; paroxetine maleate; paroxetine mesylate); sertraline (e.g., sertraline hydrochloride); sibutramine, tametraline hydrochloride; viqualine; and zimeldine (e.g., zimeldine hydrochloride).

SSRIs are drugs that inhibit 5-hydroxytryptamine (5-HT) uptake by neurons of the central nervous system. SSRIs show selectivity with respect to 5-HT over norepinephrine uptake. They are less likely than tricyclic antidepressants to cause anticholinergic side effects and are less dangerous in overdose. SSRIs, such as paroxetine, sertraline, fluoxetine, citalopram, fluvoxamine, nor₁-citalopram, venlafaxine, milnacipran, nor₂-citalopram, nor-fluoxetine, or nor-sertraline are used to treat a variety of psychiatric disorders, including depression, anxiety disorders, panic attacks, and obsessive-compulsive disorder. Dosages given here are the standard recommended doses for psychiatric disorders. In practicing the methods of the invention, effective amounts may be different.

Cericlamine

Cericlamine has the following structure:

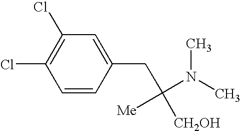

Structural analogs of cericlamine are those having the formula: 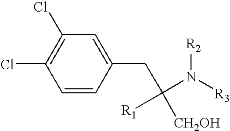
as well as pharmaceutically acceptable salts thereof, wherein R₁ is a C₁-C₄ alkyl and R₂ is H or C₁₋₄ alkyl, R₃ is H, C₁₋₄ alkyl, C₂₋₄ alkenyl, phenylalkyl or cycloalkylalkyl with 3 to 6 cyclic carbon atoms, alkanoyl, phenylalkanoyl or cycloalkylcarbonyl having 3 to 6 cyclic carbon atoms, or R₂ and R₃ form, together with the nitrogen atom to which they are linked, a heterocycle saturated with 5 to 7 chain links which can have, as the second heteroatom not directly connected to the nitrogen atom, an oxygen, a sulphur or a nitrogen, the latter nitrogen heteroatom possibly carrying a C₂₋₄ alkyl.

Exemplary cericlamine structural analogs are 2-methyl-2-amino-3-(3,4-dichlorophenyl)-propanol, 2-pentyl-2-amino-3-(3,4-dichlorophenyl)-propanol, 2-methyl-2-methylamino-3-(3,4-dichlorophenyl)-propanol, 2-methyl-2-dimethylamino-3-(3,4-dichlorophenyl)-propanol, and pharmaceutically acceptable salts of any thereof.

Citalopram

Citalopram HBr (CELEXA™) is a racemic bicyclic phthalane derivative designated (±)-1-(3-dimethylaminopropyl)-1-(4-fluorophenyl)-1,3-dihydroisobenzofuran-5-carbonitrile, HBr. Citalopram undergoes extensive metabolization; nor₁-citalopram and nor₂-citalopram are the main metabolites. By way of background, Citalopram is available in 10 mg, 20 mg, and 40 mg tablets for oral administration. CELEXA™ oral solution contains citalopram HBr equivalent to 2 mg/mL citalopram base. CELEXA™ is typically administered at an initial dose of 20 mg once daily, generally with an increase to a dose of 40 mg/day. Dose increases typically occur in increments of 20 mg at intervals of no less than one week.

Citalopram has the following structure:

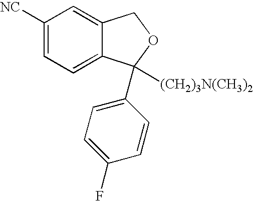

Structural analogs of citalopram are those having the formula: 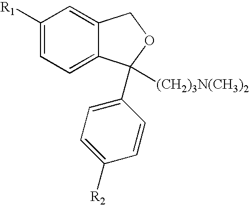
as well as pharmaceutically acceptable salts thereof, wherein each of R1 and R2 is independently selected from the group consisting of bromo, chloro, fluoro, trifluoromethyl, cyano and R—CO—, wherein R is C1-4 alkyl.

Exemplary citalopram structural analogs (which are thus SSRI structural analogs) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof.

Clovoxamine

Clovoxamine has the following structure:

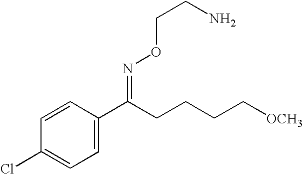

Structural analogs of clovoxamine are those having the formula: 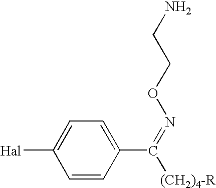
as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone O-(2-aminoethyl)oxime; and pharmaceutically acceptable salts of any thereof.

Femoxetine

Femoxetine has the following structure:

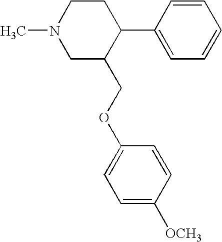

Structural analogs of femoxetine are those having the formula: 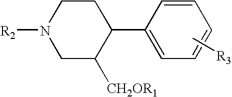
wherein R₁ represents a C₁₋₄ alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Fluoxetine

Fluoxetine hydrochloride ((±)-N-methyl-3-phenyl-3-[((alpha),(alpha),(alpha)-trifluoro-p-tolyl)oxy]propylamine hydrochloride) is sold as PROZAC™ in 10 mg, 20 mg, and 40 mg tablets for oral administration. The main metabolite of fluoxetine is nor-fluoxetine.

Fluoxetine has the following structure:

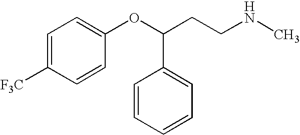

Structural analogs of fluoxetine are those compounds having the formula: 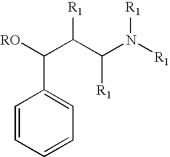
as well as pharmaceutically acceptable salts thereof, wherein each R₁ is independently hydrogen or methyl; R is naphthyl or 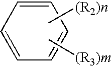
wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or β-naphthyl.

Exemplary fluoxetine structural analogs are 3-(p-isopropoxyphenoxy)-3-phenylpropylamine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropylamine bromide, N,N-dimethyl 3-(β-naphthoxy)-3-phenyl-1-methylpropylamine iodide, 3-(2′-methyl-4′,5′-dichlorophenoxy)-3-phenylpropylamine nitrate, 3-(p-t-butylphenoxy)-3-phenylpropylamine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine β-phenylpropionate, N-methyl 3-(p-iodophenoxy)-3-phenyl-propylamine propiolate, and N-methyl 3-(3-n-propylphenoxy)-3-phenyl-propylamine decanoate.

Fluvoxamine

Fluvoxamine maleate (LUVOX™) is chemically designated as 5-methoxy-4′-(trifluoromethyl) valerophenone (E)-O-(2-aminoethyl)oxime maleate. Fluvoxamine maleate is supplied as 50 mg and 100 mg tablets.

Fluvoxamine has the following structure:

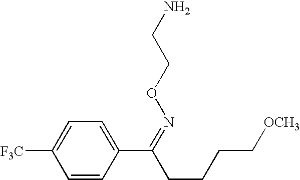

Structural analogs of fluvoxamine are those having the formula: 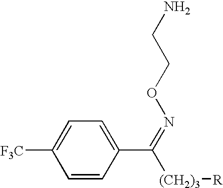
as well as pharmaceutically acceptable salts thereof, wherein R is cyano, cyanomethyl, methoxymethyl, or ethoxymethyl.

Indalpine

Indalpine has the following structure:

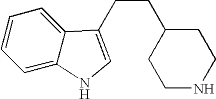

Structural analogs of indalpine are those having the formula: 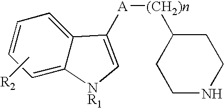
or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂— group; and n is 0, 1 or 2.

Exemplary indalpine structural analogs are indolyl-3 (piperidyl-4 methyl) ketone; (methoxy-5-indolyl-3) (piperidyl-4 methyl) ketone; (chloro-5-indolyl-3) (piperidyl-4 methyl) ketone; (indolyl-3)-1(piperidyl-4)-3 propanone, indolyl-3 piperidyl-4 ketone; (methyl-1 indolyl-3) (piperidyl-4 methyl) ketone, (benzyl-1 indolyl-3) (piperidyl-4 methyl) ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof.

Indeloxazine

Indeloxazine has the following structure:

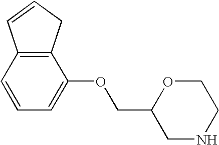

Structural analogs of indeloxazine are those having the formula: 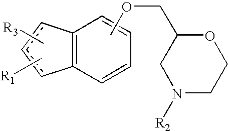
and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(1-methyl-3-phenyl-6-indenyloxymethyl)morpholine; 2-(5-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof.

Milnacipram

Milnacipram (IXEL™, Cypress Bioscience Inc.) has the chemical formula (Z)-1-diethylaminocarbonyl-2-aminoethyl-1-phenyl-cyclopropane) hydrochlorate, and is provided in 25 mg and 50 mg tablets for oral administration.

Milnacipram has the following structure:

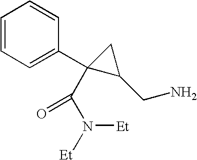

Structural analogs of milnacipram are those having the formula: 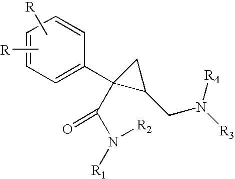
as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Paroxetine

Paroxetine hydrochloride ((−)-trans-4R-(4′-fluorophenyl)-3S-[(3′,4′-methylenedioxyphenoxy) methyl]piperidine hydrochloride hemihydrate) is provided as PAXIL™. Controlled-release tablets contain paroxetine hydrochloride equivalent to paroxetine in 12.5 mg, 25 mg, or 37.5 mg dosages. One layer of the tablet consists of a degradable barrier layer and the other contains the active material in a hydrophilic matrix.

Paroxetine has the following structure:

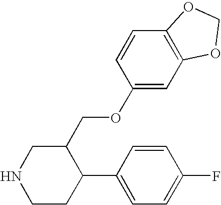

Structural analogs of paroxetine are those having the formula: 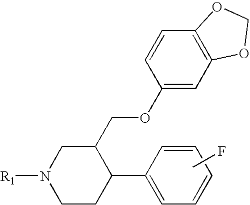
and pharmaceutically acceptable salts thereof, wherein R₁ represents hydrogen or a C₁₋₄ alkyl group, and the fluorine atom may be in any of the available positions.

Sertraline

Sertraline ((1S-cis)-4-(3,4-dichlorophenyl)-1,2,3,4-tetrahydro-N-methyl-1-nanphthalenamine hydrochloride) is provided as ZOLOFT™ in 25 mg, 50 mg and 100 mg tablets for oral administration. Because sertraline undergoes extensive metabolic transformation into a number of metabolites that may be therapeutically active, these metabolites may be substituted for sertraline in a drug combination described herein. The metabolism of sertraline includes, for example, oxidative N-demethylation to yield N-desmethylsertraline (nor-sertraline).

Sertraline has the following structure:

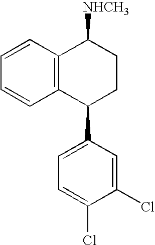

Structural analogs of sertraline are those having the formula: 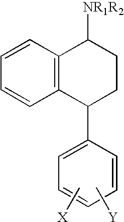
wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e. they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers.

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

Sibutramine Hydrochloride Monohydrate

Sibutramine hydrochloride monohydrate (MERIDIA™) is an orally administered agent for the treatment of obesity. Sibutramine hydrochloride is a racemic mixture of the (+) and (−) enantiomers of cyclobutanemethanamine, 1-(4-chlorophenyl)-N,N-dimethyl-(alpha)-(2-methylpropyl)-, hydrochloride, monohydrate. Each MERIDIA™ capsule contains 5 mg, 10 mg, or 15 mg of sibutramine hydrochloride monohydrate.

Zimeldine

Zimeldine has the following structure:

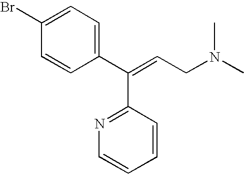

Structural analogs of zimeldine are those compounds having the formula: 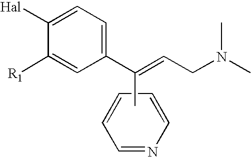
and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine 3-(4′-bromophenyl)-3-(4″-pyridyl)-dimethylallylamine; and pharmaceutically acceptable salts of any thereof.

Structural analogs of any of the above SSRIs are considered herein to be SSRI analogs and thus may be employed in any of the drug combinations described herein.

Metabolites

Pharmacologically active metabolites of any of the foregoing SSRIs can also be used in the drug combinations described herein. Exemplary metabolites are didesmethylcitalopram, desmethylcitalopram, desmethylsertraline, and norfluoxetine.

Analogs

Functional analogs of SSRIs can also be used in the drug combinations described herein. Exemplary SSRI functional analogs are provided below. One class of SSRI analogs are SNRIs (selective serotonin norepinephrine reuptake inhibitors), which include venlafaxine and duloxetine.

Venlafaxine

Venlafaxine hydrochloride (EFFEXOR™) is an antidepressant for oral administration. It is designated (R/S)-1-[2-(dimethylamino)-1-(4-methoxyphenyl)ethyl]cyclohexanol hydrochloride or (±)-1-[(alpha)-[(dimethyl-amino)methyl]-p-methoxybenzyl]cyclohexanol hydrochloride.

Venlafaxine has the following structure:

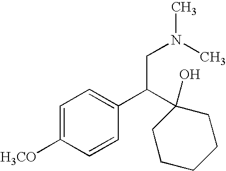

Structural analogs of venlafaxine are those compounds having the formula: 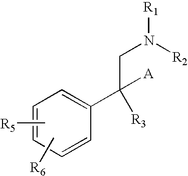
as well as pharmaceutically acceptable salts thereof, wherein A is a moiety of the formula: 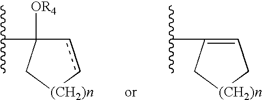
where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Duloxetine

Duloxetine has the following structure:

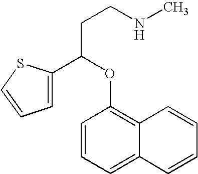

Structural analogs of duloxetine are those compounds described by the formula disclosed in U.S. Pat. No. 4,956,388, hereby incorporated by reference.

Other SSRI analogs are 4-(2-fluorophenyl)-6-methyl-2-piperazinothieno[2,3-d]pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; O-desmethylvenlafaxine; WY 45,818; WY 45,881; N-(3-fluoropropyl)paroxetine; Lu 19005; and SNRIs described in PCT Publication No. WO04/004734.

Tricyclic Antidepressants

In another embodiment, a drug combination comprises a tricyclic antidepressant (TCA) (which are described herein in detail), or a structural or functional analog thereof in combination with a non-steroidal immunophilin-dependent immunosuppressant (NsIDI). Maprotiline (brand name LUDIOMIL) is a secondary amine tricyclic antidepressant that inhibits norepinephrine reuptake and is structurally related to imipramine, a dibenzazepine. While such agents have been used for the treatment of anxiety and depression, maprotiline, for example, increases the potency of an immunosuppressive agent, and is useful as anti-inflammatory agent.

Maprotiline (brand name LUDIOMIL) and maprotiline structural analogs have three-ring molecular cores (see formula (IV), supra). These analogs include other tricyclic antidepressants (TCAs) having secondary amine side chains (e.g., nortriptyline, protriptyline, desipramine) as well as N-demethylated metabolites of TCAs having tertiary amine side chains. Preferred maprotiline structural and functional analogs include tricyclic antidepressants that are selective inhibitors of norepinephrine reuptake. Tricyclic compounds that can be used in the methods, compositions, and kits of the invention include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-[1-(4-methyl-1-piperazinyl)-5H-dibenzo(b,e)(1,4)diazepine; 4-(11H-dibenz[b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. patent application Ser. Nos. 10/617,424 or 60/504,310.

Triclosan

In another embodiment, a drug combination comprises triclosan or another phenoxy phenol, or a structural or functional analog thereof in combination with a non-steroidal immunophilin-dependent immunosuppressant (NsIDI).

Triclosan is a chloro-substituted phenoxy phenol that acts as a broad-spectrum antibiotic. We report herein that triclosan also increases the potency of immunosuppressive agents, such as cyclosporine, and is useful in the anti-inflammatory combination of the invention for the treatment of an immunoinflammatory disorder, proliferative skin disease, organ transplant rejection, or graft versus host disease. Triclosan structural analogs include chloro-substituted phenoxy phenols, such as 5-chloro-2-(2,4-dichlorophenoxy)phenol, hexachlorophene, dichlorophene, as well as other halogenated hydroxydiphenyl ether compounds. Triclosan functional analogs include clotrimazole as well as various antimicrobials such as selenium sulfide, ketoconazole, triclocarbon, zinc pyrithione, itraconazole, asiatic acid, hinokitiol, mipirocin, clinacycin hydrochloride, benzoyl peroxide, benzyl peroxide, minocyclin, octopirox, ciclopirox, erythromycin, zinc, tetracycline, azelaic acid and its derivatives, phenoxy ethanol, ethylacetate, clindamycin, meclocycline. Functional and/or structural analogs of triclosan are also described, e.g., in U.S. Pat. Nos. 5,043,154, 5,800,803, 6,307,049, and 6,503,903.

Triclosan may achieve its anti-bacterial activity by binding to and inhibiting the bacterial enzyme Fab1, which is required for bacterial fatty acid synthesis. Triclosan structural or functional analogs, including antibiotics that bind Fab1, may also be useful in the combinations of the invention.

Antihistamines

In yet another embodiment a drug combination comprises a histamine receptor antagonist (or analog thereof) and a non-steroidal immunophilin-dependent inhibitor. Antihistamines are compounds that block the action of histamine. Classes of antihistamines include the following:

(1) Ethanolamines (e.g., bromodiphenhydramine, carbinoxamine, clemastine, dimenhydrinate, diphenhydramine, diphenylpyraline, and doxylamine);

(2) Ethylenediamines (e.g., pheniramine, pyrilamine, tripelennamine, and triprolidine);

(3) Phenothiazines (e.g., diethazine, ethopropazine, methdilazine, promethazine, thiethylperazine, and trimeprazine);

(4) Alkylamines (e.g., acrivastine, brompheniramine, chlorpheniramine, desbrompheniramine, dexchlorpheniramine, pyrrobutamine, and triprolidine);

(5) piperazines (e.g., buclizine, cetirizine, chlorcyclizine, cyclizine, meclizine, hydroxyzine);

(6) Piperidines (e.g., astemizole, azatadine, cyproheptadine, desloratadine, fexofenadine, loratadine, ketotifen, olopatadine, phenindamine, and terfenadine);

(7) Atypical antihistamines (e.g., azelastine, levocabastine, methapyrilene, and phenyltoxamine).

In the drug combinations described herein, either non-sedating or sedating antihistamines may be employed. Particularly desirable antihistamines for use in the drug combinations described herein are non-sedating antihistamines such as loratadine and desloratadine. Sedating antihistamines can also be used in a drug combination. In certain embodiments, sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other suitable antihistamines include acrivastine; ahistan; antazoline; astemizole; azelastine (e.g., azelsatine hydrochloride); bamipine; bepotastine; bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine diftumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Structural analogs of antihistamines may also be used in a drug combination described herein. Antihistamine analogs include, without limitation, 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo[a,d)cyclohepten-5-ylidene)-tropane.

Other suitable compounds for use in a drug combination include AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that are suitable for use in the drug combinations described herein are described in U.S. Pat. Nos. 3,956,296; 4,254,129; 4,254,130; 4,282,833; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

Loratadine

Loratadine (CLARITIN) is a tricyclic piperidine that acts as a selective peripheral histamine H1-receptor antagonist. Loratadine and structural and functional analogs thereof, such as piperidines, tricyclic piperidines, histamine H1-receptor antagonists, are useful in a drug combination described herein.

Loratadine functional and/or structural analogs include other H1-receptor antagonists, such as AHR-11325, acrivastine, antazoline, astemizole, azatadine, azelastine, bromopheniramine, carebastine, cetirizine, chlorpheniramine, chlorcyclizine, clemastine, cyproheptadine, descarboethoxyloratadine, dexchlorpheniramine, dimenhydrinate, diphenylpyraline, diphenhydramine, ebastine, fexofenadine, hydroxyzine ketotifen, lodoxamide, levocabastine, methdilazine, mequitazine, oxatomide, pheniramine pyrilamine, promethazine, pyrilamine, setastine, tazifylline, temelastine, terfenadine, trimeprazine, tripelennamine, triprolidine, utrizine, and similar compounds (described, e.g., in U.S. Pat. Nos. 3,956,296, 4,254,129, 4,254,130, 4,283,408, 4,362,736, 4,394,508, 4,285,957, 4,285,958, 4,440,933, 4,510,309, 4,550,116, 4,692,456, 4,742,175, 4,908,372, 5,204,249, 5,375,693, 5,578,610, 5,581,011, 5,589,487, 5,663,412, 5,994,549, 6,201,124, and 6,458,958).

Loratadine, cetirizine, and fexofenadine are second-generation H1-receptor antagonists that lack the sedating effects of many first generation H1-receptor antagonists. Piperidine H1-receptor antagonists include loratadine, cyproheptadine hydrochloride (PERIACTIN), and phenindiamine tartrate (NOLAHIST). Piperazine H1-receptor antagonists include hydroxyzine hydrochloride (ATARAX), hydroxyzine pamoate (VISTARIL), cyclizine hydrochloride (MAREZINE), cyclizine lactate, and meclizine hydrochloride.

Phenothiazines

In another embodiment, the drug combination comprises a phenothiazine, or a structural or functional analog thereof, in combination with a non-steroidal immunophilin-dependent immunosuppressant (NsIDI).

Phenothiazines that are useful in the drug combinations include compounds having the general formula (VI): 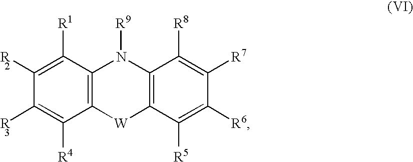
or a pharmaceutically acceptable salt thereof, wherein R² is selected from the group consisting of: CF₃, Cl, F, OCH₃, COCH₃, CN, OCF₃, COCH₂CH₃, CO(CH₂)₂CH₃, and SCH₂CH₃; R⁹ is selected from the group consisting of: 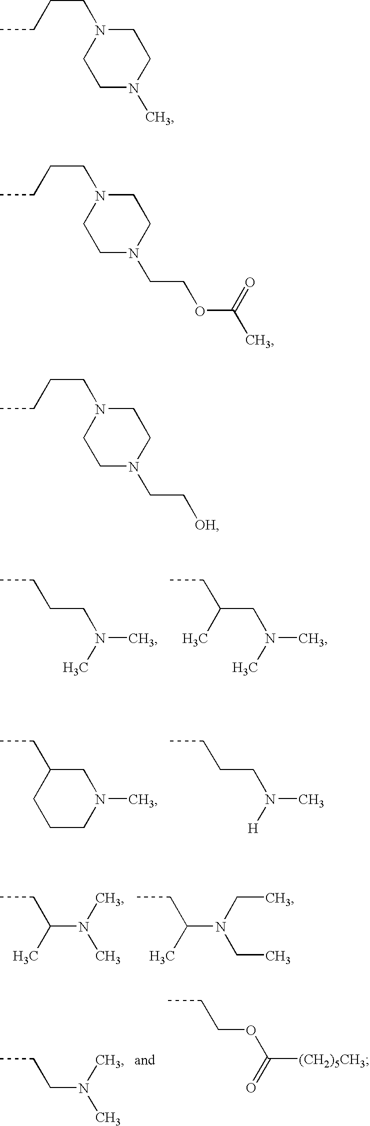
each of R¹, R³, R⁴, R⁵, R⁶, R⁷, and R⁸ is, independently, H, OH, F, OCF₃, or OCH₃; and W is selected from the group consisting of:

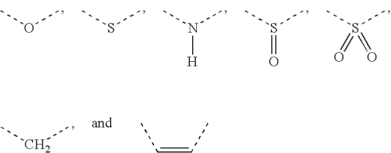

In some embodiments, the phenothiazine is a phenothiazine conjugate including a phenothiazine covalently attached via a linker to a bulky group of greater than 200 daltons or a charged group of less than 200 daltons. Such conjugates retain their anti-inflammatory activity in vivo and have reduced activity in the central nervous system in comparison to the parent phenothiazine.

Phenothiazine conjugates that are useful in drug combinations described herein include compounds having the general formula (VII).

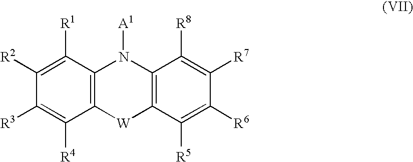

In formula (VII), R² is selected from the group consisting of: CF₃, halo, OCH₃, COCH₃, CN, OCF₃, COCH₂CH₃, CO(CH₂)₂CH₃, S(O)₂CH₃, S(O)₂N(CH₃)₂, and SCH₂CH₃; A¹ is selected from the group consisting of G¹, 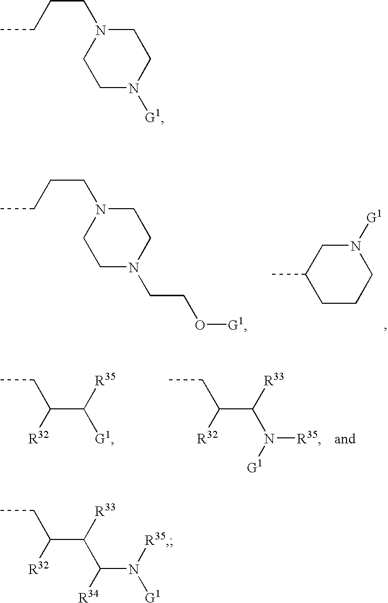
each of R¹, R³, R⁴, R⁵, R⁶, R⁷, and R⁸ is independently H, OH, F, OCF₃, or OCH₃; R³², R³³, R³⁴, and R³⁵, are each, independently, selected from H or C₁₋₆ alkyl; W is selected from the group consisting of: NO, 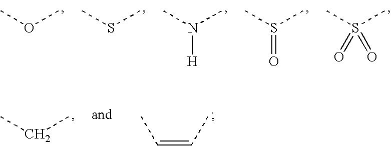
and G¹ is a bond between the phenothiazine and a linker, L.

The linker L is described by formula (VIII):
G¹-(Z¹)ₒ-(Y¹)ᵤ-(Z₂)ₛ-(R⁹)-(Z₃)ₜ-(Y²)ᵥ-(Z⁴)ₚ-G² (VIII)

In formula (VIII), G¹ is a bond between the phenothiazine and the linker, G² is a bond between the linker and the bulky group or between the linker and the charged group, each of Z¹, Z², Z³, and Z⁴ is, independently, selected from O, S, and NR³⁹; R³⁹ is hydrogen or a C₁₋₆ alkyl group; each of Y₁ and Y² is, independently, selected from carbonyl, thiocarbonyl, sulphonyl, phosphoryl or similar acid-forming groups; o, p, s, t, u, and v are each independently 0 or 1; and R⁹ is a C₁₋₁₀ alkyl, a linear or branched heteroalkyl of 1 to 10 atoms, a C₂₋₁₀ alkene, a C₂₋₁₀ alkyne, a C₅₋₁₀ aryl, a cyclic system of 3 to 10 atoms, —(CH₂CH₂O)_{q}CH₂CH₂— in which q is an integer of 1 to 4, or a chemical bond linking G¹-(Z¹)ₒ-(Y¹)ᵤ-(Z²)ₛ- to -(Z³)ₜ-(Y²)ᵥ-(Z⁴)ₚ-G².

The bulky group can be a naturally occurring polymer or a synthetic polymer. Natural polymers that can be used include, without limitation, glycoproteins, polypeptides, or polysaccharides. Desirably, when the bulky group includes a natural polymer, the natural polymer is selected from alpha-1-acid glycoprotein and hyaluronic acid. Synthetic polymers that can be used as bulky groups include, without limitation, polyethylene glycol, and the synthetic polypeptide N-hxg.

The most commonly prescribed member of the phenothiazine family is chlorpromazine, which has the structure:

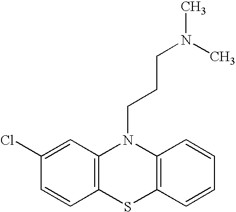

Chlorpromazine is a phenothiazine that has long been used to treat psychotic disorders. Phenothiazines include chlorpromazine functional and structural analogs, such as acepromazine, chlorfenethazine, chlorpromazine, cyamemazine, enanthate, fluphenazine, mepazine, mesoridazine besylate, methotrimeprazine, methoxypromazine, norchlorpromazine, perazine, perphenazine, prochlorperazine, promethazine, propiomazine, putaperazine, thiethylperazine, thiopropazate, thioridazine, trifluoperazine, or triflupromazine (or a salt of any of the above); and functional analogs that act as dopamine D2 antagonists (e.g., sulpride, pimozide, spiperone, clebopride, bupropion, and haloperidol).

Chlorpromazine is currently available in the following forms: tablets, capsules, suppositories, oral concentrates and syrups, and formulations for injection.

Because chlorpromazine undergoes extensive metabolic transformation into a number of metabolites that may be therapeutically active, these metabolites may be substituted for chlorpromazine in a drug combination described herein. The metabolism of chlorpromazine yields, for example, oxidative N-demethylation to yield the corresponding primary and secondary amine, aromatic oxidation to yield a phenol, N-oxidation to yield the N-oxide, S-oxidation to yield the sulphoxide or sulphone, oxidative deamination of the aminopropyl side chain to yield the phenothiazine nuclei, and glucuronidation of the phenolic hydroxy groups and tertiary amino group to yield a quaternary ammonium glucuronide. In other examples of chlorpromazine metabolites useful in the anti-inflammatory combination of the invention, each of positions 3, 7, and 8 of the phenothiazine can independently be substituted with a hydroxyl or methoxyl moiety.

Another phenothiazine is ethopropazine (brand name PARSITAN), an anticholinergic phenothiazine that is used as an antidyskinetic for the treatment of movement disorders, such as Parkinson's disease. Ethopropazine also has antihistaminic properties. Ethopropazine also increases the potency of immunosuppressive agents, such as cyclosporines. Unlike antipsychotic phenothiazines, which have three carbon atoms between position 10 of the central ring and the first amino nitrogen atom of the side chain at this position, strongly anticholinergic phenothiazines (e.g., ethopropazine, diethazine) have only two carbon atoms separating the amino group from position 10 of the central ring.

Ethopropazine structural analogs include trifluoroperazine dihydrochloride, thioridazine hydrochloride, and promethazine hydrochloride. Additional ethopropapazine structural analogs include 10-[2,3-bis(dimethylamino)propyl]phenothiazine, 10-[2,3-bis(dimethylamino)propyl]phenothiazine hydrochloride, 10-[2-(dimethylamino)propyl]phenothiazine; 10-[2-(dimethylamino)propyl]phenothiazine hydrochloride; and 10-[2-(diethylamino)ethyl]phenothiazine and mixtures thereof (see, e.g., U.S. Pat. No. 4,833,138).

Ethopropazine acts by inhibiting butyrylcholinesterase. Ethopropazine functional analogs include other anticholinergic compounds, such as Artane (trihexyphenidyl), Cogentin (benztropine), biperiden (U.S. Pat. No. 5,221,536), caramiphen, ethopropazine, procyclidine (Kemadrin), and trihexyphenidyl. Anticholinergic phenothiazines are extensively metabolized, primarily to N-dealkylated and hydroxylated metabolites. Ethopropazine metabolites may be substituted for ethopropazine in the drug combinations described herein.

Mu Opioid Receptor Agonists

In yet another embodiment, a drug combination may comprise a mu opioid receptor agonist (or analog thereof) and a non-steroidal immunophilin-dependent inhibitor. Loperamide hydrochloride (IMMODIUM) is a mu opioid receptor agonist useful in the treatment of diarrhea (U.S. Pat. No. 3,714,159). Loperamide and loperamide analogs increase the potency of an immunosuppressive agent and are useful in the treatment of an immunoinflammatory disorder, organ transplant rejection, or graft versus host disease. Loperamide is a piperidine butyramide derivative that is related to meperidine and diphenoxylate. It acts by relaxing smooth muscles and slowing intestinal motility. Other functionally and/or structurally related compounds, include meperidine, diphenoxylate, and related propanamines. Additional loperamide functional and structural analogs are described, e.g., in U.S. Pat. Nos. 4,066,654, 4,069,223, 4,072,686, 4,116,963, 4,125,531, 4,194,045, 4,824,853, 4,898,873, 5,143,938, 5,236,947, 5,242,944, 5,849,761, and 6,353,004. Loperamide functional analogs include peptide and small molecule mu opioid receptor agonists (described in U.S. Pat. No. 5,837,809). Such agents are also useful in the drug combinations described herein. Loperamide is capable of binding to opioid receptors within the intestine and altering gastrointestinal motility.

Corticosteroids

In certain embodiments, the drug combinations described herein may be used with additional therapeutic agents, including corticosteroids. One or more corticosteroid may be formulated with non-steroidal immunophilin-dependent enhancer, or analog or metabolite thereof, in a drug combination described herein. Suitable corticosteroids are described in detail herein. Corticosteroid compounds that may be included in the drug combination containing a non-steroidal immunophilin-dependent enhancer include any one of the corticosteroids described in detail herein and known in the art.

Steroid Receptor Modulators

In still other embodiments, a drug combination ma comprise a steroid receptor modulator (e.g., an antagonist or agonist) as a substitute for or in addition to a corticosteroid. Thus, in one embodiment, the drug combination comprises an NsIDI (or an analog or metabolite thereof) and an NsIDIE and, optionally, a glucocorticoid receptor modulator or other steroid receptor modulator.

Glucocorticoid receptor modulators that may used are described in U.S. Pat. Nos. 6,380,207, 6,380,223, 6,448,405, 6,506,766, and 6,570,020, U.S. Patent Application Publication Nos. 20030176478, 20030171585, 20030120081, 20030073703, 2002015631, 20020147336, 20020107235, 20020103217, and 20010041802, and PCT Publication No. WO00/66522, each of which is hereby incorporated by reference. Other steroid receptor modulators are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used in combination with a NsIDI/NsIDIE in the drug combinations described herein include, for example, A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

In one embodiment, one or more agents typically used to treat COPD may be used as a substitute for or in addition to an NSIDI in the drug combination described herein. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, a drug combination comprises a tricyclic compound and a bronchodilator.

In a certain embodiment, one or more antipsoriatic agents typically used to treat psoriasis may be used as a substitute for or in addition to an NSIDI in the drug combination described herein. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, a drug combination features the combination of a tricyclic compound and an antipsoriatic agent.

In yet another embodiment, one or more agents typically used to treat inflammatory bowel disease may be used as a substitute for or in addition to an NsIDI in the drug combinations described herein. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, a drug combination features the combination of a tricyclic compound and any of the foregoing agents.

In still another embodiment, one or more agents typically used to treat rheumatoid arthritis may be used as a substitute for or in addition to an NsIDI in the drug combination described herein. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, a drug combination features the combination of a tricyclic compound with any of the foregoing agents.

In another embodiment, one or more agents typically used to treat asthma may be used as a substitute for or in addition to an NsIDI in the drug combination described herein. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, a drug combination features the combination of a tricyclic compound and any of the foregoing agents.

An NsIDI and an NsIDIE may be combined with other compounds, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies may be useful for the treatment of inflammatory disorders or diseases in combination with other anti-cytokine agents or agents that modulate the immune response to positively treat or prevent disease, such as agents that influence cell adhesion, or biologics (i.e., agents that block the action of IL-6, IL-1, IL-2, IL-12, IL-15 or TNF (e.g., etanercept, adelimumab, infliximab, or CDP-870). Without wishing to be bound by theory, when using agents that block the effect of TNFα, a combination therapy reduces the production of cytokines, and then agents such as etanercept or infliximab act on the remaining fraction of inflammatory cytokines, providing enhanced treatment.

Accordingly, provided herein is a drug combination comprising a non-steroidal immunophilin-dependent immunosuppressant (NsIDI) and an NsIDI enhancer (NsIDIE). Such a drug combination may also exhibit a biological activity such as the capability to decrease proinflammatory cytokine secretion or production and/or to prevent or treat an inflammatory response and/or treat or prevent an immunological disease or disorder such as an inflammatory disease or disorder or an autoimmune disease or disorder. In a particular embodiment, the NsIDI is a calcineurin inhibitor; and in another particular embodiment, the calcineurin inhibitor is cyclosporine, tacrolimus, ascomycin, pimecrolimus, or ISAtx247. In another embodiment, the NsIDI is an FK506-binding protein, which in certain specific embodiments is rapamycin or everolimus. In other embodiments, the NsIDIE is a selective serotonin reuptake inhibitor (SSRI), a tricyclic antidepressant (TCA), a phenoxy phenol, an antihistamine, a phenothiazine, or a mu opioid receptor agonist. In a particular embodiment, the SSRI is selected from fluoxetine, sertraline, paroxetine, fluvoxamine, citalopram, and escitalopram. In another certain embodiment, the TCA is selected from maprotiline, nortriptyline, protriptyline, desipramine, amitriptyline, amoxapine, clomipramine, dothiepin, doxepin, desipramine, imipramine, lofepramine, mianserin, oxaprotiline, octriptyline, and trimipramine. In a particular specific embodiment, the phenoxy phenol is triclosan. In another particular embodiment, the antihistamine is selected from ethanolamines, ethylenediamines, phenothiazines, alkylamines, piperazines, piperidines, and atypical antihistamines. In another embodiment, the antihistamine is selected from desloratadine, thiethylperazine, bromodiphenhydramine, promethazine, cyproheptadine, loratadine, clemizole, azatadine, cetirizine, chlorpheniramine, dimenhydramine, diphenydramine, doxylamine, fexofenadine, meclizine, pyrilamine, and tripelennamine.

In other particular embodiments, the phenothiazine is chlorpromazine or ethopropazine. In another particular embodiment, the mu opioid receptor agonist is a piperidine butyramide derivative. In certain other embodiments, the mu opioid receptor agonist is loperamide, meperidine, or diphenoxylate. In a specific embodiment, the drug combination comprises an NSIDI that is cyclosporine (e.g., cyclosporine A) and a mu opioid receptor loperamide. In another embodiment the drug combination comprises cyclosporine and the antihistamine ethopropazine. In yet other specific embodiments, the drug combination comprises cyclosporine and any one of the following agents: chlorpromazine, loratadine, desloratadine, triclosan (a phenoxy phenol), maprotiline (a TCA), paroxetine (an SSRI), fluoxetine (an SSRI), or sertraline (an SSRI). In another specific embodiment, the NSIDI is tacrolimus (a calcineurin inhibitor) and fluvoxamine (an SSRI).

In other embodiments, the drug combination described herein further comprises a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal calcineurin inhibitor, vitamin D analog, psoralen, retinoid, or 5-amino salicylic acid. In a more particular embodiment, the NSAID is ibuprofen, diclofenac, or naproxen; and in another particular embodiment, the COX-2 inhibitor is rofecoxib, celecoxib, valdecoxib, or lumiracoxib. In still another certain embodiment, the biologic is adelimumab, etanercept, or infliximab. In another embodiment, the DMARD is methotrexate or leflunomide. In certain embodiments, xanthine is theophylline; the anticholinergic compound is ipratropium or tiotropium; the beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline; the vitamin D analog is calcipotriene or calcipotriol; the psoralen is methoxsalen; the retinoid is acitretin or tazoretene; the 5-amino salicylic acid is mesalamine, sulfasalazine, balsalazide disodium, or olsalazine sodium; and the small molecule immunomodulator is VX 702, SCIO 469, doramapimod, RO 30201195, SCIO 323, DPC 333, pranalcasan, mycophenolate, or merimepodib.

Drug Combination Comprising an Antihistamine and Additional Agents

In another embodiment, the drug combination that has anti-scarring activity comprises at least two agents, wherein at least one agent is an antihistamine, and at least one second agent is selected from a corticosteroid and any of a number of additional agents described herein.

In another embodiment, the drug combination includes an antihistamine and a corticosteroid. In certain embodiments, the antihistamine is bromodiphenhydramine, clemizole, cyproheptadine, desloratadine, loratadine, thiethylperazine maleate, or promethazine. In certain embodiments, the corticosteroid is prednisolone, cortisone, dexamethasone, hydrocortisone, methylprednisolone, fluticasone, prednisone, triamcinolone, or diflorasone. In still other embodiments, the drug combination further comprises at least one (i.e., one or more) additional compounds, including but not limited to a glucocorticoid receptor modulator, NSAID, COX-2 inhibitor, DMARD, biologic, small molecule immunomodulator, xanthine, anticholinergic compound, beta receptor agonist, bronchodilator non-steroidal immunophilin-dependent immunosuppressant, vitamin D analog, psoralen, retinoid, or 5-amino salicylic acid.

In a particular embodiment, a drug combination comprises an antihistamine and ibudilast, and in another particular embodiment, the drug combination comprises an antihistamine and rolipram. In still another specific embodiment, the drug combination comprises an antihistamine and a tetra-substituted pyrimidopyrimidine, wherein in certain embodiments, the tetra-substituted pyrimidopyrimidine is dipyridamole. In another specific embodiment, the drug combination comprises an antihistamine and a tricyclic or tetracyclic antidepressant. In other specific embodiments, the tricyclic or tetracyclic antidepressant is nortryptiline, amoxapine, or desipramine. In one embodiment, the antihistamine is not doxepin, while in another embodiment, the antidepressant is not doxepin. In yet another embodiment, a drug combination comprises an antihistamine and a selective serotonin reuptake inhibitor (SSRI). In certain embodiments, the antihistamine is selected from bromodiphenhydramine, clemizole, cyproheptadine, desloratadine, loratadine, thiethylperazine maleate, and promethazine, and the SSRI is selected from paroxetine, fluoxetine, sertraline, and citalopram.

As described in detail herein, by “corticosteroid” is meant any naturally occurring or synthetic compound characterized by a hydrogenated cyclopentanoperhydrophenanthrene ring system. Naturally occurring corticosteroids are generally produced by the adrenal cortex. Synthetic corticosteroids may be halogenated. Exemplary corticosteroids are described herein.

By “tricyclic or tetracyclic antidepressant” is meant a compound having one the formulas (I), (II), (III), or (IV), which are described in greater detail herein.

By “antihistamine” is meant a compound that blocks the action of histamine. Classes of antihistamines include but are not limited to, ethanolamines, ethylenediamine, phenothiazine, alkylamines, piperazines, and piperidines.

By “SSRI” is meant any member of the class of compounds that (i) inhibit the uptake of serotonin by neurons of the central nervous system, (ii) have an inhibition constant (Ki) of 100 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants. Exemplary SSRIs for use in the invention are fluoxetine, fluvoxamine, paroxetine, sertraline, citalopram, and venlafaxine.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which binds to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Exemplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharmaceuticals).

In one embodiment, a drug combination comprises an antihistamine (or analog thereof) and a corticosteroid. In another embodiment, a drug combination comprises an antihistamine (or analog thereof) and a tricyclic or tetracyclic antidepressant. In yet another embodiment, a drug combination comprises an antihistamine (or analog thereof) and a selective serotonin reuptake inhibitor. In still other embodiments, a drug combination comprises an antihistamine or antihistamine analog, and dipyridamole, ibudilast, and/or rolipram, or an analog of any of these compounds.

Antihistamines

As described in detail herein, antihistamines, as described herein and above, are compounds that block the action of histamine. Classes of antihistamines include the following:

(1) Ethanolamines (e.g., bromodiphenhydramine, carbinoxamine, clemastine, dimenhydrinate, diphenhydramine, diphenylpyraline, and doxylamine);

(2) Ethylenediamines (e.g., pheniramine, pyrilamine, tripelennamine, and triprolidine);

(3) Phenothiazines (e.g., diethazine, ethopropazine, methdilazine, promethazine, thiethylperazine, and trimeprazine);

(4) Alkylamines (e.g., acrivastine, brompheniramine, chlorpheniramine, desbrompheniramine, dexchlorpheniramine, pyrrobutamine, and triprolidine);

(5) piperazines (e.g., buclizine, cetirizine, chlorcyclizine, cyclizine, meclizine, hydroxyzine);

(6) Piperidines (e.g., astemizole, azatadine, cyproheptadine, desloratadine, fexofenadine, loratadine, ketotifen, olopatadine, phenindamine, and terfenadine);

(7) Atypical antihistamines (e.g., azelastine, levocabastine, methapyrilene, and phenyltoxamine).

In the drug combinations described herein, either non-sedating or sedating antihistamines may be employed. In certain embodiments, antihistamines for use in the drug combinations described herein are non-sedating antihistamines such as loratadine and desloratadine. Sedating antihistamines can also be used in a drug combination. In certain embodiments, sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the drug combinations described herein are acrivastine; ahistan; antazoline; astemizole; azelastine (e.g., azelsatine hydrochloride); bamipine; bepotastine; bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine difumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Structural analogs of antihistamines may also be used in according to the invention. Antihistamine analogs include, without limitation, 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo[a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo[b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that are suitable for use in the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that are suitable for use in the invention are described in U.S. Pat. Nos. 3,956,296; 4,254,129; 4,254,130; 4,282,833; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

Loratadine

Loratadine (CLARITIN) is a tricyclic piperidine that acts as a selective peripheral histamine H1-receptor antagonist. Loratadine and structural and functional analogs thereof, such as piperidines, tricyclic piperidines, histamine H1-receptor antagonists, may be used in the drug combinations described herein.

Loratadine functional and/or structural analogs include other H1-receptor antagonists, such as AHR-11325, acrivastine, antazoline, astemizole, azatadine, azelastine, bromopheniramine, carebastine, cetirizine, chlorpheniramine, chlorcyclizine, clemastine, cyproheptadine, descarboethoxyloratadine, dexchlorpheniramine, dimenhydrinate, diphenylpyraline, diphenhydramine, ebastine, fexofenadine, hydroxyzine ketotifen, lodoxamide, levocabastine, methdilazine, mequitazine, oxatomide, pheniramine pyrilamine, promethazine, pyrilamine, setastine, tazifylline, temelastine, terfenadine, trimeprazine, tripelennamine, triprolidine, utrizine, and similar compounds (described, e.g., in U.S. Pat. Nos. 3,956,296, 4,254,129, 4,254,130, 4,283,408, 4,362,736, 4,394,508, 4,285,957, 4,285,958, 4,440,933, 4,510,309, 4,550,116, 4,692,456, 4,742,175, 4,908,372, 5,204,249, 5,375,693, 5,578,610, 5,581,011, 5,589,487, 5,663,412, 5,994,549, 6,201,124, and 6,458,958).

Loratadine, cetirizine, and fexofenadine are second-generation H1-receptor antagonists that lack the sedating effects of many first generation H1-receptor antagonists. Piperidine H1-receptor antagonists include loratadine, cyproheptadine hydrochloride (PERIACTIN), and phenindiamine tartrate (NOLAHIST). Piperazine H1-receptor antagonists include hydroxyzine hydrochloride (ATARAX), hydroxyzine pamoate (VISTARIL), cyclizine hydrochloride (MAREZINE), cyclizine lactate, and meclizine hydrochloride.

Corticosteroids

In certain embodiments, one or more corticosteroid may be combined and formulated with an antihistamine or analog thereof in a drug combination described herein. Various antihistamines in combination with various corticosteroids are more effective in suppressing TNFα in vitro than either agent alone. Corticosteroids are described in detail herein and suitable corticosteroids for use in combination with an anti-histamine include any one of the corticosteroid compounds described herein.

Steroid Receptor Modulators

Steroid receptor modulators (e.g., antagonists and agonists) may be used as a substitute for or in addition to a corticosteroid in the drug combinations described herein. Thus, in one embodiment, the invention features the combination of a tricyclic compound and a glucocorticoid receptor modulator or other steroid receptor modulator.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the methods, compositions, and kits of the invention A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

Ibudilast

In one embodiment, a drug combination comprises an antihistamine and ibudilast. Among the biological activities of such a drug combination includes the capability to suppress TNFα in vitro more effectively than either agent alone.

Ibudilast, or an ibudilast analog, has a structure of formula (IX).

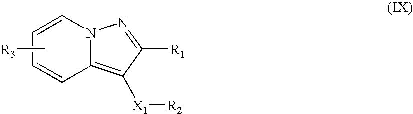

In formula (IX) R₁ and R₂ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₃ is selected from H, halide, alkoxy, and C₁₋₄ alkyl; X₁ is selected from C═O, C═N—NH—R₄, C═C(R₅)—C(O)—R₆, C═CH═CH—C(O)—R₆, and C(OH)—R₇; R₄ is selected from H and acyl; R₅ is selected from H, halide, and C₁₋₄ alkyl; R₆ is selected from OH, alkoxy and amido; and R₇ is selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. Compounds of formula (IX) include, the compounds described in U.S. Pat. Nos. 3,850,941; 4,097,483; 4,578,392; 4,925,849; 4,994,453; and 5,296,490. Commercially available compounds of formula (IX) include ibudilast and KC-764.

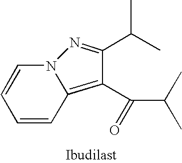

KC-764 (CAS 94457-09-7) is reported to be a platelet aggregation inhibitor.

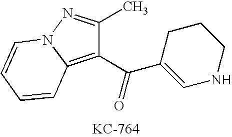

KC-764 and other compound of formula (IX) can be prepared using the synthetic methods described in U.S. Pat. Nos. 3,850,941; 4,097,483; 4,578,392; 4,925,849; 4,994,453; and 5,296,490.

Rolipram

In another embodiment, a drug combination comprises an antihistamine, or an analog thereof, and rolipram (4-[3-(cyclopentyloxy)-4-methoxyphenyl]-2-pyrrolidone) or an analog of rolipram. Rolipram analogs are described by formula (I) of U.S. Pat. No. 4,193,926, hereby incorporated by reference.

Tetra-Substituted Pyrimidopyrimidines

In another embodiment, a drug combination is provided that comprises an antihistamine, or analog thereof, in combination with a tetra-substituted pyrimidopyrimidine such as dipyridamole.

A tetra-substituted pyrimidopyrimidine comprises a structure having the formula (V) as described in detail herein. Exemplary tetra-substituted pyrimidopyrimidines that are useful in the drug combinations and methods described herein include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine); mopidamole; dipyridamole monoacetate; NU3026 (2,6-di-(2,2-dimethyl-1,3-dioxolan-4-yl)-methoxy-4,8-d]-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. No. 3,031,450, hereby incorporated by reference.

Tricyclic and Tetracyclic Antidepressants

In another embodiment, the drug combination comprises an antihistamine or antihistamine analog in combination with tricyclic and tetracyclic antidepressants and their analogs.

In one embodiment of the invention, an antihistamine or analog thereof is administered or formulated with a tricyclic or tetracyclic antidepressant, or an analog thereof. By “tricyclic or tetracyclic antidepressant analog” is meant a compound having one the formulas (I), (II), (III), or (IV), which are described in detail herein.

Tricyclic or tetracyclic antidepressants, as well as analogs thereof, that are suitable for use in the drug combinations described herein include 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo[b,e)(1,4)diazepin-11-one; 2-(2-(7-hydroxy-4-dibenzo(b,f)(1,4)thiazepine-11-yl-1-piperazinyl)ethoxy)ethanol; 2-chloro-[1-(4-methyl-1-piperazinyl)-5H-dibenzo[b,e)(1,4)diazepine; 4-(1H-dibenz[b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; 8-chloro-2-methoxy-11-(4-methyl-1-piperazinyl)-5H-dibenzo(b,e)(1,4)diazepine; (Z)-2-butenedioate; 7-hydroxyamoxapine; 8-hydroxyamoxapine; 8-hydroxyloxapine; Adinazolam; Amineptine; amitriptyline; amitriptylinoxide, amoxapine; butriptyline; clomipramine; clothiapine; clozapine; demexiptiline; desipramine; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; 11-(4-methyl-1-piperazinyl)-dibenzo(b,f)(1,4)thiazepine; dibenzepin; dimetacrine; dothiepin; doxepin; fluacizine; fluperlapine; imipramine; imipramine N-oxide; iprindole lofepramine; loxapine; loxapine hydrochloride; loxapine succinate; maprotiline; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; nortriptyline; noxiptilin; octriptyline; opipramol; oxaprotiline; perlapine; pizotyline; propizepine; protriptyline; quetiapine; quinupramine; tianeptine; tomoxetine; and trimipramine. Others are described in U.S. Pat. Nos. 4,933,438 and 4,931,435.

Selective Serotonin Reuptake Inhibitors

In another embodiment, a drug combination provided herein comprises an antihistamine or analog thereof in combination with any one of a number of SSRI compounds, or analog thereof, described herein and available in the art.

As described herein, suitable SSRIs and SSRI analogs include 1,2,3,4-tetrahydro-N-methyl-4-phenyl-1-naphthylamine hydrochloride, 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; cericlamine; citalopram; xitalopram hydrobromide; CP 53261; didesmethylcitalopram; escitalopram; escitalopram oxalate; femoxetine, fluoxetine; fluoxetine hydrochloride; fluvoxamine; fluvoxamine maleate; indalpine, indeloxazine hydrochloride, Lu 19005; milnacipran; monodesmethylcitalopram; N-(3-fluoropropyl)paroxetine; norfluoxetine; O-desmethylvenlafaxine; paroxetine; paroxetine hydrochloride; paroxetine maleate; sertraline; sertraline hydrochloride; tametraline hydrochloride; venlafaxine; venlafaxine hydrochloride; WY 45,818; WY 45,881, and zimeldine. Other SSRI or SSRI analogs useful in the methods and compositions of the invention are described in U.S. Pat. Nos. 3,912,743; 4,007,196; 4,136,193; 4,314,081; and 4,536,518, each hereby incorporated by reference.

Citalopram

Citalopram HBr (CELEXA™) is a racemic bicyclic phthalane derivative designated (±)-1-(3-dimethylaminopropyl)-1-(4-fluorophenyl)-1,3-dihydroisobenzofuran-5-carbonitrile, HBr. Citalopram undergoes extensive metabolization; nor₁-citalopram and nor₂-citalopram are the main metabolites. Citalopram is available in 10 mg, 20 mg, and 40 mg tablets for oral administration. CELEXA™ oral solution contains citalopram HBr equivalent to 2 mg/mL citalopram base. CELEXA™ is typically administered at an initial dose of 20 mg once daily, generally with an increase to a dose of 40 mg/day. Dose increases typically occur in increments of 20 mg at intervals of no less than one week.

Citalopram has the following structure:

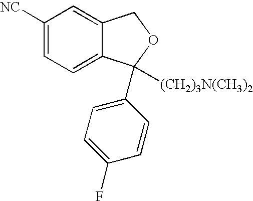

Structural analogs of citalopram are those having the formula: 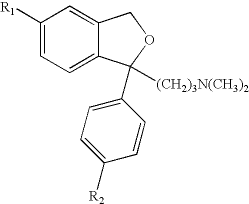
as well as pharmaceutically acceptable salts thereof, wherein each of R₁ and R₂ is independently selected from the group consisting of bromo, chloro, fluoro, trifluoromethyl, cyano and R—CO—, wherein R is C₁₋₄ alkyl.

Exemplary citalopram structural analogs (which are thus SSRI structural analogs according to the invention) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof.

Clovoxamine

Clovoxamine has the following structure:

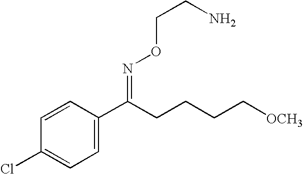

Structural analogs of clovoxamine are those having the formula: 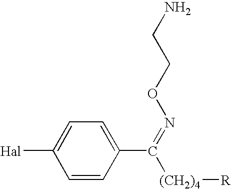
as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone O-(2-aminoethyl)oxime; and pharmaceutically acceptable salts of any thereof.

Femoxetine

Femoxetine has the following structure:

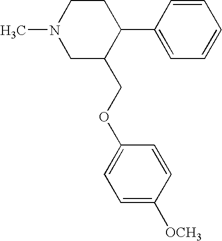

Structural analogs of femoxetine are those having the formula: 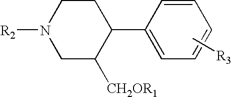
wherein R₁ represents a C₁₋₄ alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Fluoxetine

Fluoxetine hydrochloride ((±)-N-methyl-3-phenyl-3-[((alpha),(alpha),(alpha)-trifluoro-p-tolyl)oxy]propylamine hydrochloride) is sold as PROZAC™ in 10 mg, 20 mg, and 40 mg tablets for oral administration. The main metabolite of fluoxetine is nor-fluoxetine. By way of background, fluoxetine hydrochloride is typically administered as an oral solution equivalent to 20 mg/5 mL of fluoxetine. A delayed release formulation contains enteric-coated pellets of fluoxetine hydrochloride equivalent to 90 mg of fluoxetine. A dose of 20 mg/day, administered in the morning, is typically recommended as the initial dose. A dose increase may be considered after several weeks if no clinical improvement is observed.

Fluoxetine has the following structure:

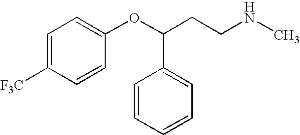

Structural analogs of fluoxetine are those compounds having the formula: 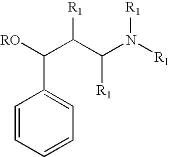
as well as pharmaceutically acceptable salts thereof, wherein each R₁ is independently hydrogen or methyl; R is naphthyl or 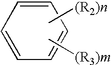
wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or β-naphthyl.

Exemplary fluoxetine structural analogs are 3-(p-isopropoxyphenoxy)-3-phenylpropylamine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropylamine bromide, N,N-dimethyl 3-(β-naphthoxy)-3-phenyl-1-methylpropylamine iodide, 3-(2′-methyl-4′,5′-dichlorophenoxy)-3-phenylpropylamine nitrate, 3-(p-t-butylphenoxy)-3-phenylpropylamine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine β-phenylpropionate, N-methyl 3-(p-iodophenoxy)-3-phenyl-propylamine propiolate, and N-methyl 3-(3-n-propylphenoxy)-3-phenyl-propylamine decanoate.

Fluvoxamine

Fluvoxamine maleate (LUVOX™) is chemically designated as 5-methoxy-4′-(trifluoromethyl) valerophenone (E)-O-(2-aminoethyl)oxime maleate. By way of background, fluvoxamine maleate is supplied as 50 mg and 100 mg tablets. Treatment for approved indications is typically initiated at 50 mg given once daily at bedtime, and then increased to 100 mg daily at bedtime after a few days, as tolerated. The effective daily dose usually lies between 100 and 200 mg, but may be administered up to a maximum of 300 mg.

Fluvoxamine has the following structure:

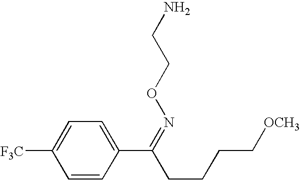

Structural analogs of fluvoxamine are those having the formula: 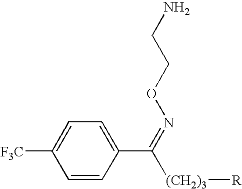
as well as pharmaceutically acceptable salts thereof, wherein R is cyano, cyanomethyl, methoxymethyl, or ethoxymethyl.

Indalpine

Indalpine has the following structure:

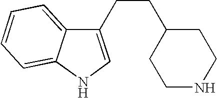

Structural analogs of indalpine are those having the formula: 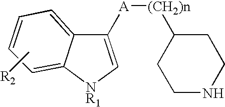
or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂— group; and n is 0, 1 or 2.

Exemplary indalpine structural analogs are indolyl-3 (piperidyl-4 methyl) ketone; (methoxy-5-indolyl-3) (piperidyl-4 methyl) ketone; (chloro-5-indolyl-3) (piperidyl-4 methyl) ketone; (indolyl-3)-1(piperidyl-4)-3 propanone, indolyl-3 piperidyl-4 ketone; (methyl-1 indolyl-3) (piperidyl-4 methyl) ketone, (benzyl-1 indolyl-3) (piperidyl-4 methyl) ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof.

Indeloxazine

Indeloxazine has the following structure:

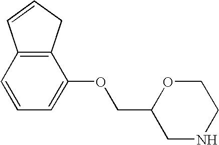

Structural analogs of indeloxazine are those having the formula: 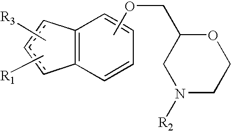
and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(1-methyl-3-phenyl-6-indenyloxymethyl)morpholine; 2-(5-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof.

Milnacipram

Milnacipram (IXEL™, Cypress Bioscience Inc.) has the chemical formula (Z)-1-diethylaminocarbonyl-2-aminoethyl-1-phenyl-cyclopropane) hydrochlorate, and is provided in 25 mg and 50 mg tablets for oral administration. By way of background, milnacipram is typically administered in dosages of 25 mg once a day, 25 mg twice a day, or 50 mg twice a day for the treatment of severe depression.

Milnacipram has the following structure:

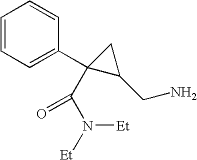

Structural analogs of milnacipram are those having the formula: 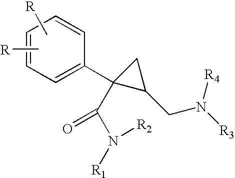
as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Paroxetine

Paroxetine hydrochloride ((−)-trans-4R-(4′-fluorophenyl)-3S-[(3′,4′-methylenedioxyphenoxy) methyl]piperidine hydrochloride hemihydrate) is currently provided as PAXIL™. Controlled-release tablets contain paroxetine hydrochloride equivalent to paroxetine in 12.5 mg, 25 mg, or 37.5 mg dosages.

Paroxetine has the following structure:

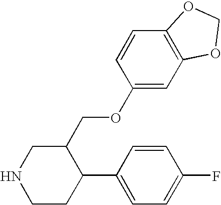

Structural analogs of paroxetine are those having the formula: 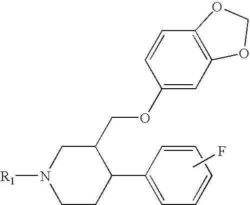
and pharmaceutically acceptable salts thereof, wherein R₁ represents hydrogen or a C₁₋₄ alkyl group, and the fluorine atom may be in any of the available positions.

Sertraline

Sertraline ((1S-cis)-4-(3,4-dichlorophenyl)-1,2,3,4-tetrahydro-N-methyl-1-nanphthalenamine hydrochloride) is provided as ZOLOFT™ in 25 mg, 50 mg and 100 mg tablets for oral administration. Because sertraline undergoes extensive metabolic transformation into a number of metabolites that may be therapeutically active, these metabolites may be substituted for sertraline in a drug combination described herein. The metabolism of sertraline includes, for example, oxidative N-demethylation to yield N-desmethylsertraline (nor-sertraline). ZOLOFT is typically administered at a dose of 50 mg once daily.

Sertraline has the following structure:

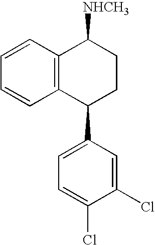

Structural analogs of sertraline are those having the formula: 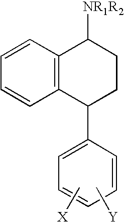
wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e. they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers.

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

Sibutramine Hydrochloride Monohydrate

Sibutramine hydrochloride monohydrate (MERIDIA™) is an orally administered agent for the treatment of obesity. Sibutramine hydrochloride is a racemic mixture of the (+) and (−) enantiomers of cyclobutanemethanamine, 1-(4-chlorophenyl)-N,N-dimethyl-(alpha)-(2-methylpropyl)-, hydrochloride, monohydrate. Each MERIDIA™ capsule contains 5 mg, 10 mg, or 15 mg of sibutramine hydrochloride monohydrate.

Zimeldine

Zimeldine has the following structure:

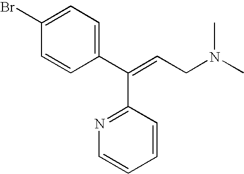

Structural analogs of zimeldine are those compounds having the formula: 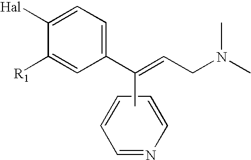
and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine; 3-(4′-bromophenyl)-3-(4″-pyridyl)-dimethylallylamine; and pharmaceutically acceptable salts of any thereof.

Structural analogs of any of the above SSRIs are considered herein to be SSRI analogs and thus may be used in any of the drug combinations described herein.

Metabolites

Pharmacologically active metabolites of any of the foregoing SSRIs can also be used in the drug combinations described herein. Exemplary metabolites are didesmethylcitalopram, desmethylcitalopram, desmethylsertraline, and norfluoxetine.

Analogs

Functional analogs of SSRIs can also be used in drug combinations described herein. Exemplary SSRI functional analogs are provided below. One class of SSRI analogs includes SNRIs (selective serotonin norepinephrine reuptake inhibitors), which include venlafaxine, duloxetine, and 4-(2-fluorophenyl)-6-methyl-2-piperazinothieno[2,3-d]pyrimidine.

Venlafaxine

Venlafaxine hydrochloride (EFFEXOR™) is an antidepressant for oral administration. It is designated (R/S)-1-[2-(dimethylamino)-1-(4-methoxyphenyl)ethyl]cyclohexanol hydrochloride or (±)-1-[(alpha)-[(dimethyl-amino)methyl]-p-methoxybenzyl]cyclohexanol hydrochloride. Compressed tablets contain venlafaxine hydrochloride equivalent to 25 mg, 37.5 mg, 50 mg, 75 mg, or 100 mg venlafaxine.

Venlafaxine has the following structure:

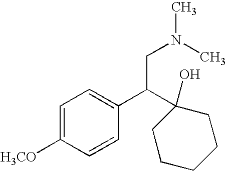

Structural analogs of venlafaxine are those compounds having the formula: 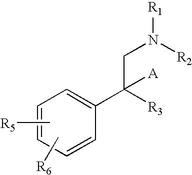
as well as pharmaceutically acceptable salts thereof, wherein A is a moiety of the formula: 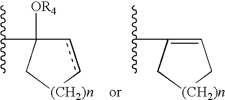
where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Duloxetine

Duloxetine has the following structure:

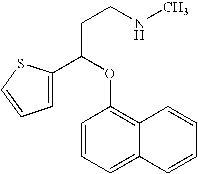

Structural analogs of duloxetine are those compounds described by the formula disclosed in U.S. Pat. No. 4,956,388, hereby incorporated by reference.

Other SSRI analogs are 4-(2-fluorophenyl)-6-methyl-2-piperazinothieno[2,3-d]pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; O-desmethylvenlafaxine; WY 45,818; WY 45,881; N-(3-fluoropropyl)paroxetine; Lu 19005; and SNRIs described in PCT Publication No. WO04/004734.

Other Compounds

In certain embodiments, the drug combinations described herein comprise one or more compounds selected from methotrexate, hydroxychloroquine, sulfasalazine, tacrolimus, sirolimus, mycophenolate mofetil, and methyl prednisolone.

Nonsteroidal Immunophilin-Dependent Immunosuppressants

In another embodiment, a drug combination comprises an antihistamine and a nonsteroidal immunophilin-dependent immunosuppressant (NsIDI).

In one embodiment, the NsIDI is cyclosporine. In another embodiment, the NsIDI is tacrolimus. In another embodiment, the NsIDI is rapamycin. In another embodiment, the NsIDI is everolimus. In still other embodiments, the NsIDI is pimecrolimus or the NsIDI is a calcineurin-binding peptide. Two or more NsIDIs can be administered contemporaneously. Calcineurin inhibitors including cyclosporines, tacrolimus, pimecrolimus, and rapamycin are described in detail herein. In another embodiment, a drug combination comprises an antihistamine and a peptide moiety. Peptide moieties, including peptides, peptide mimetics, peptide fragments, either natural, synthetic or chemically modified, that impair the calcineurin-mediated dephosphorylation and nuclear translocation of NFAT that may be used in the drug combinations described herein are described in detail above.

In certain embodiments, the drug combination further comprising at least one other compound, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Other agents—either biologics or small molecules—that modulate an immune response may also be included in a drug combination. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-6, IL-1, IL-2, IL-12, IL-15 or TNFα. Agents that inhibit TNFα include etanercept, adelimumab, infliximab, and CDP-870. Small molecule immunodulators include, for example, p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib.

In another embodiment, one or more agents typically used to treat COPD may be used as a substitute for or in addition to a corticosteroid in the drug combinations described herein. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, a drug combination features the combination of a tricyclic compound and a bronchodilator.

In another embodiment, one or more antipsoriatic agents typically used to treat psoriasis may be used as a substitute for or in addition to a corticosteroid in the drug combinations described herein. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, a drug combination features the combination of a tricyclic compound and an antipsoriatic agent.

In still another embodiment, one or more agents typically used to treat inflammatory bowel disease may be used as a substitute for or in addition to a corticosteroid in the drug combinations described herein. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, a drug combination features the combination of a tricyclic compound and any of the foregoing agents.

In still another embodiment, one or more agents typically used to treat rheumatoid arthritis may be used as a substitute for or in addition to a corticosteroid in the drug combinations described herein. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, a drug combination features the combination of a tricyclic compound with any of the foregoing agents.

In yet another embodiment, one or more agents typically used to treat asthma may be used as a substitute for or in addition to a corticosteroid in the drug combinations described herein. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, a drug combination features the combination of a tricyclic compound and any of the foregoing agents.

In one embodiment, a drug combination is provided that comprises an antihistamine or an antihistamine analog and a corticosteroid. In certain embodiments, the antihistamine is bromodiphenhydramine, clemizole, cyproheptadine, desloratadine, loratadine, thiethylperazine maleate, epinastine, or promethazine. In certain other embodiments, the corticosteroid is prednisolone, cortisone, dexamethasone, hydrocortisone, methylprednisolone, fluticasone, prednisone, triamcinolone, or diflorasone. In a particular embodiment, the antihistamine is desloratadine or loratadine and the corticosteroid is prednisolone. In other specific embodiments, the drug combination comprises prednisolone and any one of the anti-histamine compounds, bromodiphenhydramine, clemizole, cyproheptadine, thiethylperazine maleate, and promethazine.

In other certain embodiments, the drug combination comprises amoxapine (tricyclic compound) and any one of the antihistamine compounds bromodiphenhydramine, loratadine, cyproheptadine, desloratadine, clemizole, thiethylperazine maleate, and promethazine. In another embodiment, the drug combination comprises nortryptyline (tricyclic or tetracyclic antidepressant) and any one of the antihistamine compounds bromodiphenhydramine, loratadine, cyproheptadine, desloratadine, clemizole, thiethylperazine maleate, and promethazine. In another specific embodiment, the drug combination comprises paroxetine (an SSRI) and any one of the antihistamine compounds bromodiphenhydramine, loratadine, cyproheptadine, desloratadine, clemizole, thiethylperazine maleate, and promethazine. In still another specific embodiment, the drug combination comprises fluoxetine (an SSRI) and any one of the antihistamine compounds bromodiphenhydramine, loratadine, cyproheptadine, desloratadine, clemizole, thiethylperazine maleate, and promethazine. In one specific embodiment, the drug combination comprises setraline (an SSRI) and any one of the antihistamine compounds clemizole, desloratadine, and promethazine. In still another specific embodiment, the drug combination comprises despiramine and any one of the antihistamine compounds loratadine, clemizole, desloratadine, and promethazine.

In still other embodiments, prednisolone is combined with any one of the antihistamine compounds, azatidine, bromodiphenhydramine, cetrizine, chlorpheniramine, clemizole, cyproheptadine, desloratadine, dimenhydrinate, doxylamine, fexofenadine, loratadine, meclizine, promethazine, pyrilamine, thiethylperazine; and tripelennamine. In another specific embodiment, the drug combination comprises prednisolone and epinastine; in another specific embodiment, the drug combination comprises prednisolone and cyproheptadine.

In another embodiment, the drug combination comprises dipyridamole (a tetra substituted pyrimiodpyrimidine) and an anti-histamine, which is any one of bromodiphenhydramine, cyproheptadine, loratadine, and thiethylperazine.

In other embodiments, the drug combination may further comprise a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal immunophilin-dependent immunosuppressant, vitamin D analog, psoralen, retinoid, or 5-amino salicylic acid. In certain embodiments, the NSAID is ibuprofen, diclofenac, or naproxen. In other certain particular embodiments, the COX-2 inhibitor is rofecoxib, celecoxib, valdecoxib, or lumiracoxib. In another particular embodiment, the biologic is adelimumab, etanercept, or infliximab; and in another particular embodiment, the DMARD is methotrexate or leflunomide. In other particular embodiments, the xanthine is theophylline, and in other certain embodiments, the anticholinergic compound is ipratropium or tiotropium. In still another certain embodiment, the beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline. In another certain embodiment, the vitamin D analog is calcipotriene or calcipotriol; and in other certain embodiments, the psoralen is methoxsalen. In one certain embodiment, the retinoid is acitretin or tazoretene. In another specific embodiment, the 5-amino salicylic acid is mesalamine, sulfasalazine, balsalazide disodium, or olsalazine sodium. In still another specific embodiment, the small molecule immunomodulator is VX 702, SCIO 469, doramapimod, RO 30201195, SCIO 323, DPC 333, pranalcasan, mycophenolate, or merimepodib.

In another embodiment, a drug combination comprises an antihistamine or an antihistamine analog and ibudilast or an analog thereof. In a particular embodiment, the antihistamine is bromodiphenhydramine, clemizole, cyproheptadine, desloratadine, loratadine, thiethylperazine maleate, epinastine, or promethazine. In a specific embodiment, the drug combination comprises (i) desloratadine or loratadine and (ii) ibudilast. In another specific embodiment, the drug combination comprises bromodiphenhydramine and ibudilast; in another embodiment, the drug combination comprises cyproheptadine and ibudilast; and in still another embodiment, the drug combination comprises thiethylperazine maleate and idublast. In certain embodiments, the drug combination further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

In one embodiment, the drug combination comprises an antihistamine or an antihistamine analog and rolipram or an analog thereof. In a particular embodiment, the antihistamine is bromodiphenhydramine, clemizole, cyproheptadine, desloratadine, loratadine, thiethylperazine maleate, epinastine, or promethazine. In a particular embodiment, the drug combination comprises desloratadine or loratadine and rolipram. In another specific embodiment, the drug combination comprises bromodiphenhydramine and rolipram; in another embodiment, the drug combination comprises cyproheptadine and rolipram; and in still another embodiment, the drug combination comprises thiethylperazine maleate and rolipram. In certain embodiments, the drug combination further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

In another embodiment, the drug combination comprises an antihistamine or an antihistamine analog and a tetra-substituted pyrimidopyrimidine. In a certain embodiment, the antihistamine is bromodiphenhydramine, clemizole, cyproheptadine, desloratadine, loratadine, thiethylperazine maleate, epinastine, or promethazine. In a specific embodiment, the tetra-substituted pyrimidopyrimidine is dipyridimole. In another specific embodiment, the antihistamine is desloratadine or loratadine and the tetra-substituted pyrimidopyrimidine is dipyridimole. In another specific embodiment, the drug combination may further comprise an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

In one embodiment, the drug combination comprises an antihistamine or an antihistamine analog and a tricyclic or tetracyclic antidepressant or analog thereof. In a particular embodiment, the antihistamine is bromodiphenhydramine, clemizole, cyproheptadine, desloratadine, loratadine, thiethylperazine maleate, epinastine, or promethazine. In another particular embodiment, the tricyclic antidepressant is nortryptiline, amoxapine, or desipramine. In one specific embodiment, the drug combination comprises clemizole and nortryptiline, and in another specific embodiment, the drug combination comprises clemizole and amoxapine. In another embodiment, the drug combination further comprises an NSAID, COX-2 inhibitor, biologic, small molecule immunomodulator, DMARD, xanthine, anticholinergic compound, beta receptor agonist, bronchodilator, non-steroidal immunophilin-dependent immunosuppressant, vitamin D analog, psoralen, retinoid, or 5-amino salicylic acid.

In still another embodiment, the drug combination comprises an antihistamine or an antihistamine analog and an SSRI or analog thereof. In certain embodiments, the antihistamine is bromodiphenhydramine, clemizole, cyproheptadine, desloratadine, loratadine, thiethylperazine maleate, epinastine, or promethazine. In other certain embodiments, the SSRI is paroxetine or fluoxetine. In another particular embodiment, the drug combination further comprises a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal immunophilin-dependent immunosuppressant, vitamin D analog, psoralen, retinoid, or 5-amino salicylic acid.

In yet another specific embodiment, the drug combination comprises desloratadine and cyclosporine, and in another specific embodiment, the drug combination comprises loratadine and cyclosporine.

Drug Combination Comprising a Triazole and an Aminopyridine

In certain embodiments, the drug combination that has anti-scarring activity comprises at least two agents, wherein at least one agent is a triazole compound and at least one second agent is an aminopyridine compound. In specific embodiments, the triazole is fluconazole or itraconazole and the aminopyridine is a diaminopyridine such as phenazopyridine (PZP).

Compounds useful in the invention include those described herein in any of their pharmaceutically acceptable forms, including isomers such as diastereomers and enantiomers, salts, solvates, and polymorphs thereof, as well as racemic mixtures of the compounds described herein.

Triazole Compounds

By “triazole” is meant any member of the class of anti-fungal compounds having a five-membered ring of two carbon atoms and three nitrogen atoms. A compound is considered “anti-fungal” if it inhibits growth of a species of fungus by at least 25%. Exemplary triazoles include, for example, fluconazole, terconazole, itraconazole, posaconazole (SCH 56592), ravuconazole (BMS 207147), and voriconazole (UK-109,496), the structures of which are depicted in Table 1.

TABLE 1
Exemplary Triazole Compounds

| Name of | |
|---|---|
| Triazole | Structure |
| fluconazole | 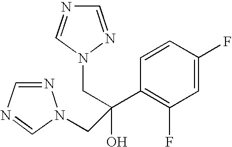 |
| itraconazole | 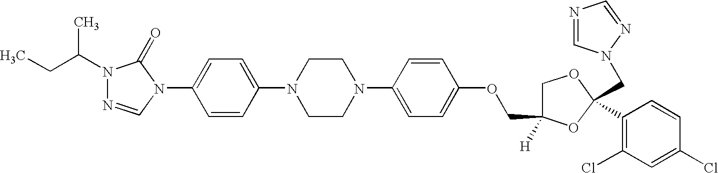 |
| terconazole | 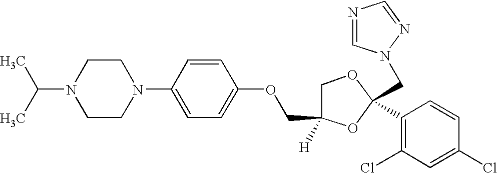 |
| posaconazole | 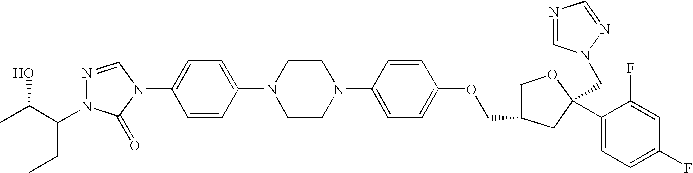 |
| ravuconazole | 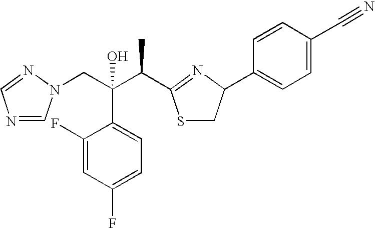 |
| voriconazole | 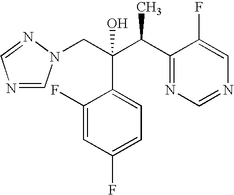 |

Aminopyridine Compounds

By “aminopyridine” is meant any pyridine ring-containing compound in which the pyridine has one, two, or three amino group substitutents. Other substitutents may optionally be present. Exemplary aminopyridines include, for example, phenazopyridine, 4-aminopyridine, 3,4-diaminopyridine, 2,5-diamino-4-methylpyridine, 2,3,6-triaminopyridine, 2,4,6-triaminopyridine, and 2,6-diaminopyridine, the structures of which are depicted in the Table 2.

TABLE 2
Exemplary Aminopyridine Compounds

| Aminopyridine Name | Structure |
|---|---|
| Phenazopyridine | 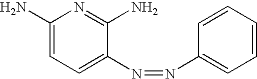 |
| 4-aminopyridine | 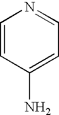 |
| 3,4-diaminopyridine | 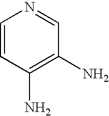 |
| 2,5-diamino-4-methylpyridine | 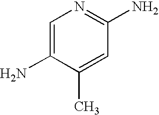 |
| 2,3,6-triazminopyridine | 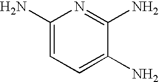 |
| 2,4,6-triaminopyridine | 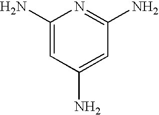 |
| 2,6-diaminopyridine | 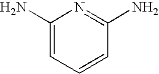 |

Compounds useful in the drug combination include those described herein in any of their pharmaceutically acceptable forms, including isomers such as diastereomers and enantiomers, salts, solvates, and polymorphs thereof, as well as racemic mixtures of the compounds described herein.

In certain embodiments, a drug combination comprises a triazole and an aminopyridine. In certain embodiments, the triazole is fluconazole, terconazole, itraconazole, voriconizole, posuconizole, or ravuconazole; in a certain specific embodiment, the triazole is fluconazole. In other certain embodiments, the aminopyridine is phenazopyridine, 4-amino-pyridine; 3,4-diaminopyridine; 2,5-diamino-4-methylpyridine; 2,3,6-triaminopyridine; 2,4,6-triaminopyridine; or 2,6-diaminopyridine; in a certain specific embodiment, the aminopyridine is phenazopyridine. In a specific embodiment, the triazole is fluconazole and the aminopyridine is phenazopyridine. In certain other embodiments, the triazole is itraconazole and the aminopyridine is phenazopyridine.

Drug Combination Comprising an Antiprotozoal Agent and an Aminopyridine and a Drug Combination Comprising an Antiprotozoal Agent and a Quaternary Ammonium Compound

In certain embodiments, the drug combination that has anti-scarring activity comprises at least two agents, wherein at least one agent is an antiprotozoal agent and at least one second agent is an aminopyridine compound. In one specific embodiment, the antiprotozoal agent is pentamidine and the aminopyridine compound is a diaminopyridine such as phenazopyridine (PZP). In another embodiment, the drug combination that has anti-scarring activity comprises at least two agents, wherein at least one agent is an antiprotozoal agent and at least one second agent is a quaternary ammonium compound. In one specific embodiment, the antiprotozoal agent is pentamidine and the quaternary ammonium compound is pentolinium.

Antiprotozoal Agents

In one embodiment, an antiprotozoal agent is pentamidine or a pentamidine analog. Aromatic diamidino compounds can replace pentamidine in the anti-fungal combination of the invention. Aromatic diamidino compounds such as propamidine, butamidine, heptamidine, and nonamidine exhibit similar biological activities as pentamidine in that they exhibit antipathogenic or DNA binding properties. Other analogs (e.g., stilbamidine and indole analogs of stilbamidine, hydroxystilbamidine, diminazene, benzamidine, 4,4′-(pentamethylenedioxy)phenamidine, dibrompropamidine, 1,3-bis(4-amidino-2-methoxyphenoxy)propane (DAMP), netropsin, distamycin, phenamidine, amicarbalide, bleomycin, actinomycin, and daunorubicin) also exhibit properties similar to those of pentamidine.

In one embodiment, the antiprotozoal agent has the following structure having the formula (X): 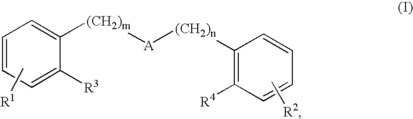
or a pharmaceutically acceptable salt thereof,
wherein A is 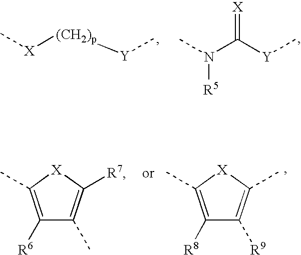
wherein each of X and Y is, independently, O, NR¹⁰, or S, each of R⁵ and R¹⁰ is independently, H or C₁-C₆ alkyl, each of R⁶, R⁷, R⁸, and R⁹ is, independently, H, C₁-C₆ alkyl, halogen, C₁-C₆ alkyloxy, C₆-C₁₈ aryloxy, or C₆-C₁₈ aryl-C₁-C₆ alkyloxy, p is an integer between 2 and 6, inclusive, each of m and n is, independently, an integer between 0 and 2, inclusive, each of R¹ and R² is 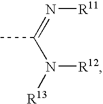
wherein R¹² is H, C₁-C₆ alkyl, C₁-C₈ cycloalkyl, C₁-C₆alkyloxy-C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 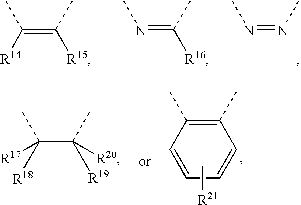
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ is, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, each of R³ and R⁴ is, independently, H, Cl, Br, OH, OCH₃, OCF₃, NO₂, and NH₂, or R³ and R⁴ together form a single bond.

In a related aspect, in the compound of formula (X), A is 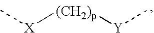
each of X and Y is independently O or NH, p is an integer between 2 and 6, inclusive, and m and n are, independently, integers between 0 and 2, inclusive, wherein the sum of m and n is greater than 0; or A is 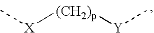
each of X and Y is independently O or NH, each of m and n is 0, and each of R¹ and R² is, independently, selected from the group represented by 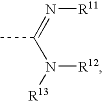
wherein R¹² is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl C₁-C₆ alkoxy), carbo(C₆-C₁₈ aryloxy), or C₆-C₁₈ aryl, and R¹¹ is H, OH, or C₁-C₆ alkyloxy, or R¹¹ and R¹² together represent 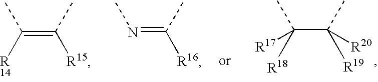
wherein each of R¹⁴, R¹⁵, and R¹⁶ is, independently, H, C₁-C₆ alkyl, halogen, or trifluoromethyl, each of R¹⁷, R¹⁸, and R¹⁹ is, independently, H or C₁-C₆ alkyl, and R²⁰ is C₁-C₆ alkyl, C₁-C₆ alkyloxy, or trifluoromethyl; or A is 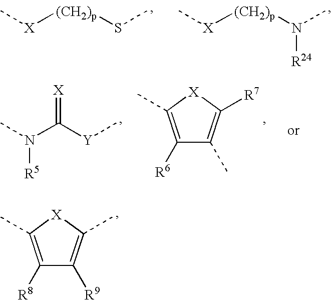
each of X and Y is, independently, O, NR¹⁰, or S, each of R⁵ and R¹⁰ is, independently, H or C₁-C₆ alkyl, each of R⁶, R⁷, R⁸, and R⁹ is, independently, H, C₁-C₆ alkyl, halogen, C₁-C₆ alkyloxy, C₆-C₁₈ aryloxy, or C₆-C₁₈ aryl C₁-C₆ alkyloxy, R²⁴ is C₁-C₆ alkyl, p is an integer between 2 and 6, inclusive, each of m and n is, independently, an integer between 0 and 2, inclusive, each of R¹ and R² is, independently, selected from the group represented by 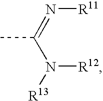
wherein R¹² is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent 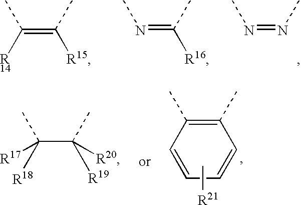
wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl.

Other analogs include stilbamidine (A-1) and hydroxystilbamidine (A-2), and their indole analogs (e.g., A-3).

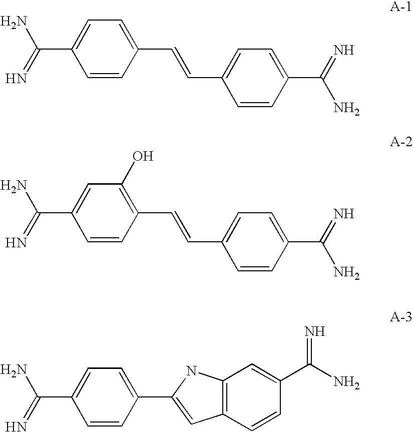

Each amidine moiety in A-1, A-2, or A-3 may be replaced with one of the moieties depicted in formula (X) above as

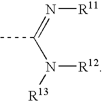

As is the case for pentamidine, salts of stilbamidine and its related compounds are also useful in the method of the invention. Preferred salts include, for example, dihydrochloride and methanesulfonate salts.

Still other analogs include the bis-benzamidoximes described in U.S. Pat. Nos. 5,723,495, 6,214,883, 6,025,398, and 5,843,980. Other diamidine analogs have also been described in U.S. Pat. Nos. 5,578,631, 5,428,051, 5,602,172, 5,521,189, 5,686,456, 5,622,955, 5,627,184, 5,606,058, 5,643,935, 5,792,782, 5,939,440, 5,639,755, 5,817,686, 5,972,969, 6,046,226, 6,156,779, 6,294,565, 5,817,687, 6,017,941, 6,172,104, and 6,326,395 each of which is herein incorporated by reference. Any of the amidine and diamidine analogs described in the foregoing patents can be used in a combination of the invention.

Exemplary analogs are 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorene, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan. Methods for making any of the foregoing compounds are described in U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, an U.S. Patent Application Publication Nos. US 2001/0044468 A1 and US 2002/0019437 A1.

Exemplary compounds having formula (X) include but are not limited to pentamidine, propamidine, butamidine, heptamidine, nonamidine, stilbamidine, hydroxystilbamidine, diminazene, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime. In specific embodiments, the compound of formula (X) is pentamidine, 2,5-bis(4-amidinophenyl)furan, or 2,5-bis(4-amidinophenyl)furan-bis-O-methylamidoxime.

As described herein a drug combination comprising an anti-protozoal agent may comprise an aromatic diamidine, which includes the following exemplary compounds: pentamidine, propamidine, butamidine, heptamidine, nonamidine, stilbamidine, hydroxystilbamidine, diminazene, benzamidine, phenamidine, dibrompropamidine, or any one of the pentamidine analogues described herein.

The structure of pentamidine is:

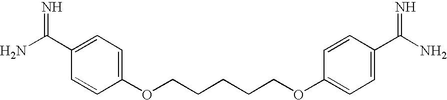

Pentamidine isethionate is a white, crystalline powder soluble in water and glycerin and insoluble in ether, acetone, and chloroform. Pentamidine is chemically designated 4,4′-diamidino-diphenoxypentane di(β-hydroxyethanesulfonate). The molecular formula is C₂₃H₃₆N₄O₁₀S₂ and the molecular weight is 592.68.

Recently, pentamidine was shown to be an effective inhibitor of protein tyrosine phosphatase 1B (PTP1B). Because PTP1B dephosphorylates and inactivates Jak kinases, which mediate signaling of cytokines with leishmanicidal activity, its inhibition by pentamidine might result in augmentation of cytokine signaling and anti-leishmania effects. Pentamidine has also been shown to be a potent inhibitor of the oncogenic phosphatases of regenerating liver (PRL). Pentamidine has also been shown to inhibit the activity of endo-exonuclease (PCT Publication No. WO01/35935). Thus, in the methods of the invention, pentamidine can be replaced by any PTP1B inhibitor, PRL inhibitor, or endo-exonuclease inhibitor.

Pentamidine metabolites are also useful in the anti-fungal combination of the invention. Pentamidine is rapidly metabolized in the body to at least seven primary metabolites. Some of these metabolites share one or more activities with pentamidine. It is likely that some pentamidine metabolites will have anti-fungal activity when administered in combination with an antiproliferative agent. Seven pentamidine metabolites (B-1 through B-7) are shown below. 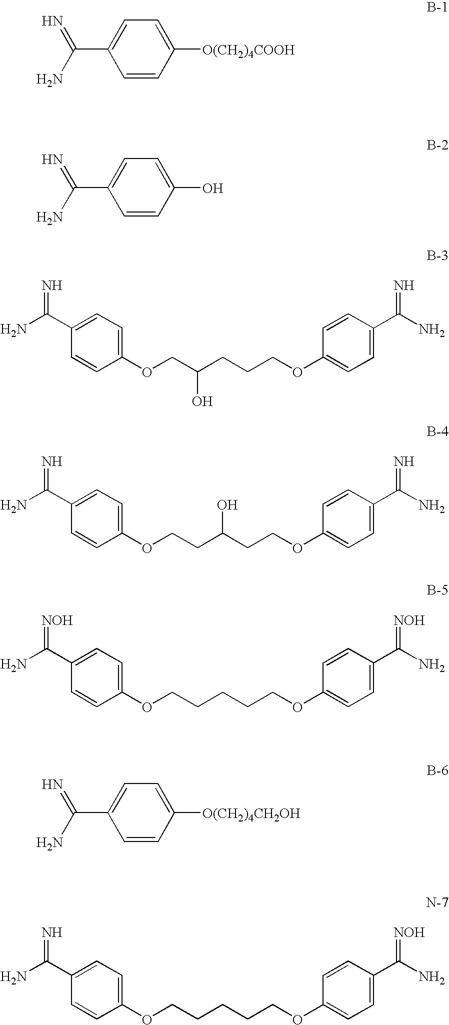
Aminopyridine Compounds

By “aminopyridine” is meant any pyridine ring-containing compound in which the pyridine has one, two, or three amino group substitutents. Other substitutents may optionally be present.

In one embodiment, the aminopyridine agent has a structure of the formula (XI): 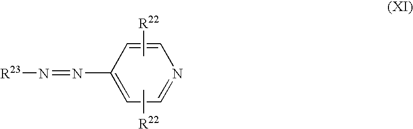
wherein each R²² is, independently, NH₂, H, OH, a halide, C₁₋₁₀ alkyl, C₁₋₁₀ alkoxyalkyl, hydroxyalkyl (wherein the alkyl group has from 1 to 10 carbon atoms), aminoalkyl (wherein the alkyl group has from 1 to 10 carbon atoms), C₁₋₁₀ alkylaminoalkyl, cycloalkyl (wherein the alkyl group has from 1 to 10 carbon atoms), aryl, or C₁₋₁₀ alkylaryl; and R²³ is NH₂, H, OH, a halide, C₁₋₁₀ alkyl, C₁₋₁₀ alkoxyalkyl, hydroxyalkyl (wherein the alkyl group has from 1 to 10 carbon atoms), aminoalkyl (wherein the alkyl group has from 1 to 10 carbon atoms), C₁₋₁₀ alkylaminoalkyl, cycloalkyl (wherein the alkyl group has from 1 to 10 carbon atoms), aryl, or C₁₋₁₀ alkylaryl.

In one embodiment, the aminopyridine agent has the following structure having the compound having the formula (XII): 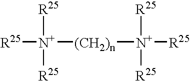
wherein each R²⁵ is, independently, NH₂, H, OH, a halide, C₁₋₁₀ alkyl, C₁₋₁₀ alkoxyalkyl, hydroxyalkyl (wherein the alkyl group has from 1 to 10 carbon atoms), aminoalkyl (wherein the alkyl group has from 1 to 10 carbon atoms), C₁₋₁₀ alkylaminoalkyl, cycloalkyl (wherein the alkyl group has from 1 to 10 carbon atoms), C₆₋₁₈ aryl, or C₁₋₁₀ alkylaryl; n is an integer between 2 and 10, inclusive.
Phenazopyridine

By “aminopyridine” is meant any pyridine ring-containing compound in which the pyridine has one, two, or three amino group substitutents. Other substitutents may optionally be present. Aminopyridines include phenazopyridine (C-1), 4-aminopyridine (C-2), 3,4-diaminopyridine (C-3), 2,5-diamino-4-methylpyridine (C-4), 2,3,6-triaminopyridine (C-5), 2,4,6-triaminopyridine (C-6), and 2,6-diaminopyridine (C-7), the structures of which are depicted below.

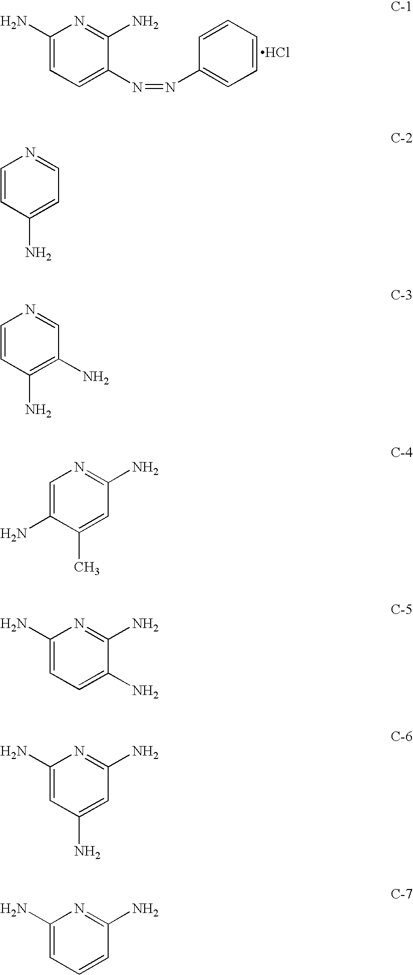

Aminopyridines can accommodate many modifications while still maintaining structural and therapeutic efficacy. Phenazopyridine and derivatives thereof have been disclosed in U.S. Pat. Nos. 1,680,108, 1,680,109, 1,680,110, and 1,680,111. Heterocyclic azo derivatives and N-substituted diaminopyridines have also been described (see, e.g., U.S. Pat. Nos. 2,145,579 and 3,647,808).

Aminopyridine compounds exhibit anti-fungal activity. Additional compounds that exhibit anti-fungal activity that may be included in the drug combination described herein include fluconazole, amphotericin B, nystatin, pimaricin, ketoconazole, miconazole, thiabendazole, emlkonazole, itraconazole, ravuconazole, posaconazole, voriconazole, dapsone, griseofulvin, carbol-fuchsin, clotrimazole, econazole, haloprogin, mafenide, naftifine, oxiconazole, silver sulfadiazine, sulconazole, terbinafine, amorolfine, tioconazole, tolnaftate, undecylenic acid, butoconazle, gentian violet, terconazole, flucytosine, ciclopirox, caspofungin acetate, micafungin, and V-echinocandin (LY303366).

Quaternary Ammonium Compounds

By “quaternary ammonium compound” is meant any quaternary ammonium-containing compound in which the nitrogen atom has four group substitutents. Quaternary ammonium compounds may be mono-, symmetrical quaternary, or asymmetrical quaternary compounds.

Quaternary ammonium compounds include, for example, pentolinium (D-1), hexamethonium (D-2), pentamethonium (D-3), tetraethylammonium (D-4), tetramethylammonium (D-5), chlorisondamine (D-6), and trimethaphan (D-7), the structures of which are depicted below.

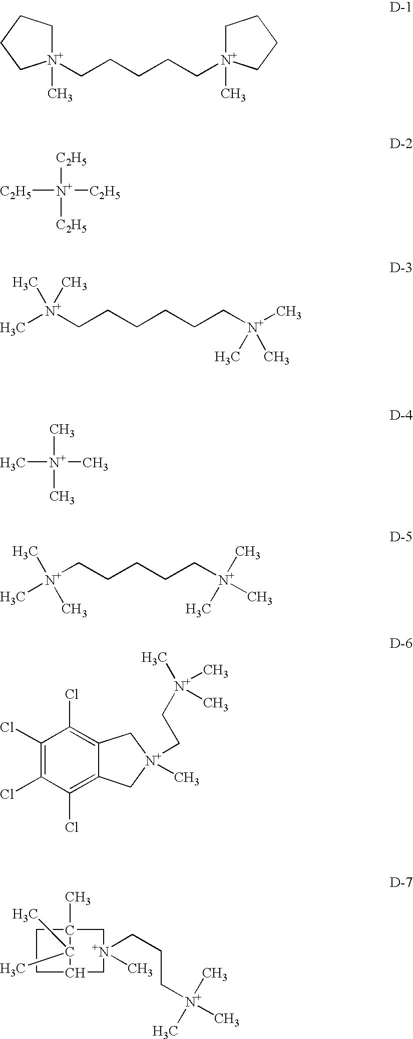

Pentolinium (pentamethylene-1,5-bis(N-methylpyrrolidinium) and its salt, pentolinium ditartrate, are symmetrical quaternary ammonium compounds. The tartrate salt form of pentolinium has the molecular formula C₂₃H₄₂N₂O₁₂ with a molecular weight of 538.6. Pentolinium ditartrate is a white powder, near odorless, and highly soluble in water.

Pentolinium Analogs

Quaternary ammonium compounds can accommodate many modifications while still maintaining structural and therapeutic efficacy. Pentolinium and its derivatives thereof are described in U.S. Pat. Nos. 4,902,720 and 6,096,788, each of which is herein incorporated by reference. Any of the quaternary ammonium compounds described in the foregoing patents can be used in a combination herein.

Compounds useful in the invention include those described herein in any of their pharmaceutically acceptable forms, including isomers such as diastereomers and enantiomers, salts, solvates, and polymorphs, thereof, as well as racemic mixtures of the compounds described herein.

In certain embodiments, the drug combination comprises (i) an aromatic diamidine or a compound having formula (X); and at least one of (ii) an aminopyridine; (iii) a quaternary ammonium compound; or (iv) a compound having one of formulas (XI) and (XII). In particular embodiments, aromatic diamidines suitable for use in the drug combinations described herein include pentamidine, propamidine, butamidine, heptamidine, nonamidine, stilbamidine, hydroxystilbamidine, diminazene, benzamidine, 4,4′-(pentamethylenedioxy) di-, dihydrochloride, phenamidine, dibrompropamidine, 1,3-bis(4-amidino-2-methoxyphenoxy)propane, netropsin, distamycin, and phenamidine. Aminopyridines suitable for use drug combinations described herein include phenazopyridine, 4-amino-pyridine, 3,4-diaminopyridine, 2,5-diamino-4-methylpyridine, 2,3,6-triaminopyridine, 2,4,6-triaminopyridine, and 2,6-diaminopyridine. Quaternary ammonium compounds suitable for the drug combinations described herein include pentolinium, hexamethonium, pentamethonium, tetramethylammonium, tetraethylammonium, trimethaphan, and chlorisondamine. In a specific embodiment, the drug combination comprises the aromatic diamidine pentamidine and phenazopyridine (aminopyridine). In another specific embodiment, the drug combination comprises pentamidine and the quaternary ammonium compound pentolinium.

In other embodiments, the drug combination may further comprise an anti-fungal agent wherein the anti-fungal agent is selected from amphotericin B, fluconazole, nystatin, pimaricin, ketoconazole, miconazole, thiabendazole, emlkonazole, itraconazole, ravuconazole, posaconazole, voriconazole, dapsone, griseofulvin, carbol-fuchsin, clotrimzole, econazole, haloprogin, mafenide, naftifine, oxiconazole, silver sulfadiazine, sulconazole, terbinafine, amorolfine, tioconazole, tolnaftate, undecylenic acid, butoconazle, gentian violet, terconazole, flucytosine, ciclopirox, caspofungin acetate, micafungin, and V-echinocandin (LY303366).

Drug Combination Comprising an Aromatic Diamidine and an Antiestrogen, Anti-Fungal Imidazole, Disulfuram, or Ribavirin

In certain embodiments, the drug combination that has anti-scarring activity comprises at least two agents, wherein at least one agent is an aromatic diamidine compound and at least one second agent is selected from an antiestrogen, an anti-fungal imidazole, disulfuram, and ribavirin. In a particular embodiment, an aromatic diamidine includes pentamidine, propamidine, butamidine, heptamidine, nonamidine, stilbamidine, hydroxystilbamidine, diminazene, benzamidine, 4,4′-(pentamethylenedioxy) di-, dihydrochloride, phenamidine, dibrompropamidine, 1,3-bis(4-amidino-2-methoxyphenoxy) propane, netropsin, distamycin, and phenamidine. In a specific embodiment, the aromatic diamidine is pentamidine. In other certain embodiments, an antiestrogen includes tamoxifen, 4-hydroxy tamoxifen, clomifene, raloxifene, and faslodex. In a specific embodiment, the antiestrogen is tamoxifen. In another particular embodiment, an anti-fungal imidazole compound includes ketoconazole, sulconazole, clotrimazole, econazole, miconazole, oxiconazole, tioconazole, and butoconazole. In a specific embodiment, the anti-fungal imidazole compound is ketoconazole or sulconazole. In certain specific embodiments, the drug combination comprises pentamidine and disulfuram; in another specific embodiment, the drug combination comprises pentamidine and ketoconazole; in still another specific embodiment, the drug combination comprises pentamidine and ribavirin; in yet another specific embodiment, the drug combination comprises pentamidine and sulconazole; and in still another specific embodiment, the drug combination comprises pentamidine and tamoxifen.

Aromatic diamidine compounds are described in detail herein and any one of these described compounds may be included in the drug combinations described herein. Particularly, pentamidine, pentamidine analogs, aromatic diamidine compounds comprising a structure having the formula (X); pentamidine metabolites (B-1 through B-7) are described. Other analogs include stilbamidine (A-1) and hydroxystilbamidine (A-2), and their indole analogs (e.g., A-3) and are also described in detail herein. Exemplary compounds having a structure of formula (X) and exemplary compounds that are pentamidine analogs are also provided herein.

Pentamidine Analogs

In addition, to the pentamidine analogs described above, pentamidine analogs include the following. Aromatic diamidino compounds can replace pentamidine in the antiproliferative combinations of the invention. Aromatic diamidines such as propamidine, butamidine, heptamidine, and nonamidine share properties with pentamidine in that they exhibit antipathogenic or DNA binding properties. Other analogs (e.g., stilbamidine and indole analogs of stilbamidine, hydroxystilbamidine, diminazene, benzamidine, 4,4′-(pentamethylenedioxy) di-, dihydrochloride, dibrompropamidine, 1,3-bis(4-amidino-2-methoxyphenoxy)propane (DAMP), netropsin, distamycin, phenamidine, amicarbalide, bleomycin, actinomycin, and daunorubicin) also exhibit properties similar to those of pentamidine.

Certain pentamidine analogs are described, for example, by formula (XIII). 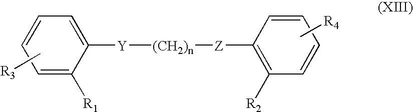
wherein each of Y and Z is, independently, O or N; each of R₁ and R₂ is, independently, NH₂, H, OH, a halide, C₁₋₅ alkyl, C₁₋₅ alkoxyalkyl, hydroxyalkyl (wherein the alkyl group has from 1 to 5 carbon atoms), aminoalkyl (wherein the alkyl group has from 1 to 5 carbon atoms), C₁₋₅ alkylaminoalkyl, cycloalkyl (wherein the alkyl group has from 1 to 5 carbon atoms), aryl, or C₁₋₅ alkylaryl; and n is an integer from 2 to 6, inclusive; and each of R₃ and R₄ is, independently, at the meta- or para-position and is selected from the group consisting of: 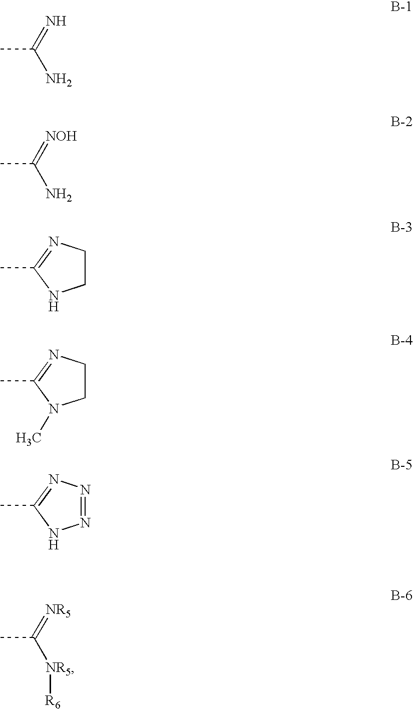
wherein each of R₅ and R₆ is, independently, NH₂, H, OH, a halide, C₁₋₅ alkyl, C₁₋₅ alkoxyalkyl, hydroxyalkyl (wherein the alkyl group has from 1 to 5 carbon atoms), aminoalkyl (wherein the alkyl group has from 1 to 5 carbon atoms), C₁₋₅ alkylaminoalkyl, cycloalkyl (wherein the alkyl group has from 1 to 5 carbon atoms), aryl, or C₁₋₅ alkylaryl.
Anti-Estrogenic Compounds

By “antiestrogen” or “antiestrogenic compound” is meant any agent that blocks an activity of estrogen. These agents may act to competitively or non-competitively inhibit the binding of estrogen to one of its receptors. Certain antiestrogens selectively bind to an estrogen receptor and inhibit the binding of estrogen to the receptor. Binding of the antiestrogens to the ERs may induce structural change in the engaged ER to inhibit DNA binding, dimerization, protein-protein interactions, or ER nuclear localization.

Exemplary antiestrogenic compounds are tamoxifen (K-1), 4-hydroxy tamoxifen (K-4), clomifene (K-2), raloxifene (K-5), and faslodex (ICI 182,780; K-3), the structures of which, are depicted below.

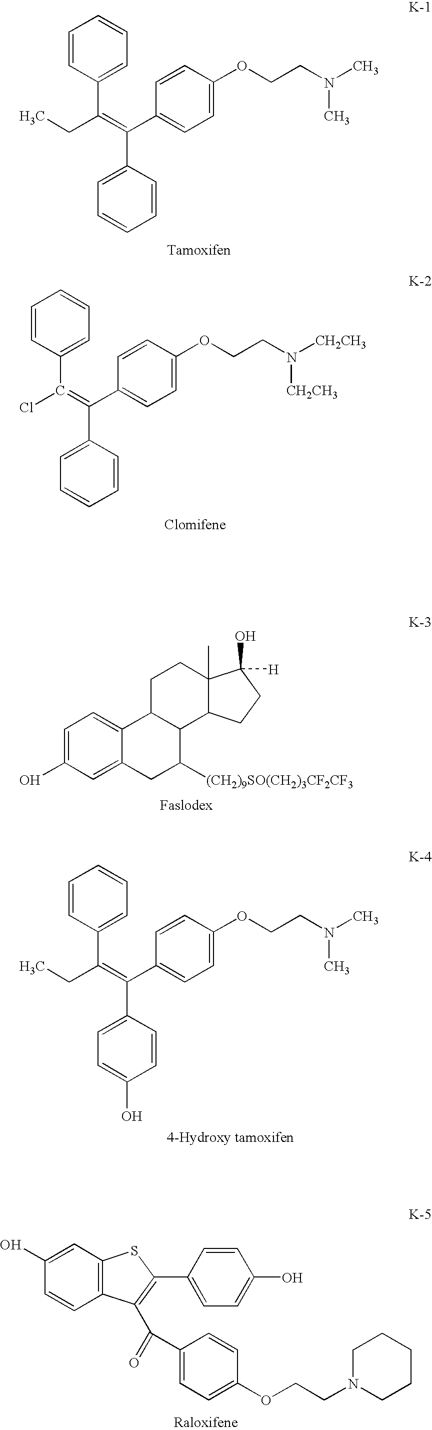

Tamoxifen is a non-steroidal estrogen antagonist, used alone or as an adjunct to surgery and/or radiation therapy for the treatment of breast cancer. Tamoxifen is prepared as a citrate salt for oral administration. Tamoxifen citrate is a fine, white crystalline powder, with a solubility of 0.5 mg/mL in water and a pKₐ of 8.85. Tamoxifen metabolites include N-desmethyltamoxifen and 4-hydroxy tamoxifen is also observed.

Anti-Fungal Imidazoles

One biological activity of the imidazole family of anti-fungal agents works is inhibition of cytochrome P450 14-α-demethylase in fungal cells. This enzyme is involved in the conversion of lanosterol to ergosterol, which is the major sterol found in fungal cell membranes. The structures of suitable imidazole anti-fungal compounds are presented below.

Ketoconazole and sulconazole are two synthetic anti-fungal imidazoles. Ketoconazole is a white to slightly beige powder and is essentially insoluble in water. Ketoconazole has pKₐs of 2.9 and 6.5.

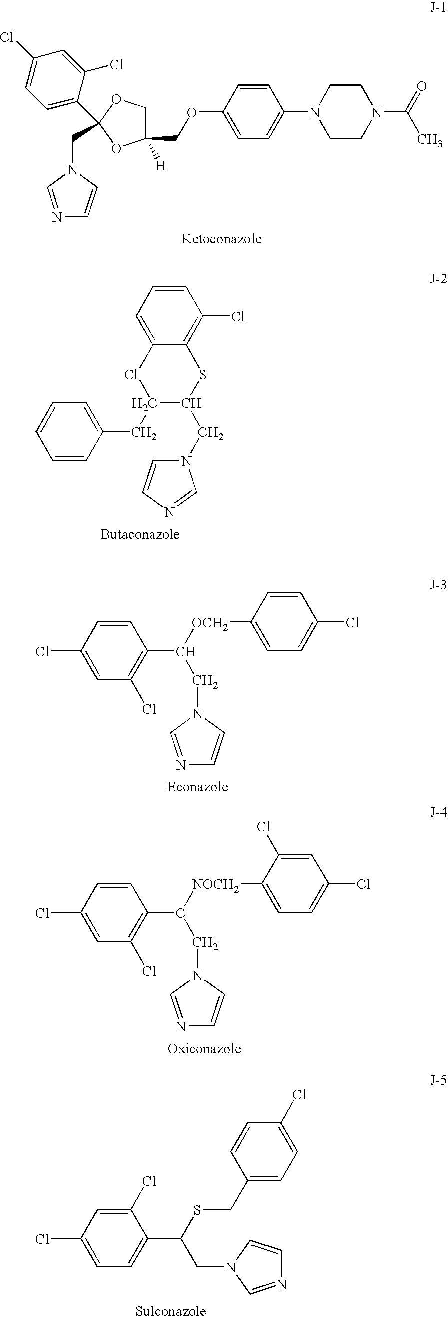

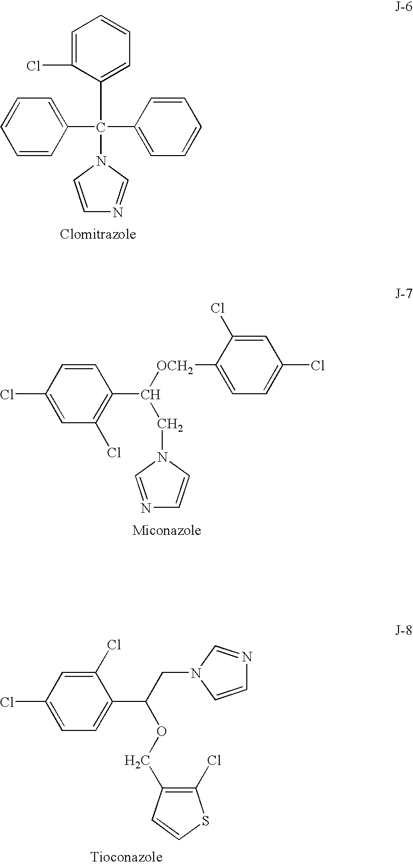

Disulfuram, more commonly known as Antabuse®, is commonly used in the treatment of alcoholism. This drug inhibits the enzyme-mediated step of acetaldehyde metabolism to acetate during alcohol catabolism.

Ribavirin is a synthetic nucleoside analog resembling guanosine. This drug is used as an anti-viral agent, blocking nucleotide synthesis and subsequently viral replication. Ribavirin inhibits both RNA and DNA virus replication. Ribavirin may be obtained as a white crystalline powder that is both odorless and tasteless. This drug is soluble in water (142 mg/mL), but only slightly soluble in alcohol.

Drug Combination Comprising an Aminopyridine and a Phenothiazine, Dacarbazine, or Phenelzine

In certain embodiments, the drug combination that has anti-scarring activity comprises at least two agents, wherein at least one agent is an aminopyridine and at least one second agent is selected from a phenothiazine compound, dacarbazine, and phenelzine. In certain specific embodiments, aminopyridines include phenazopyridine, 4-amino-pyridine, 3,4-diaminopyridine, 2,5-diamino-4-methylpyridine, 2,3,6-triaminopyridine, 2,4,6-triaminopyridine, and 2,6-diaminopyridine. In a particular embodiment, the aminopyridine is phenazopyridine. In certain specific embodiments, phenothiazines include perphenazine, chlorpromazine, prochlorperazine, mepazine, methotrimeprazine, acepromazine, thiopropazate, perazine, propiomazine, putaperazine, thiethylperazine, methopromazine, chlorfenethazine, cyamemazine, enanthate, trifluoperazine, thioridazine, and norchlorpromazine. In a particular embodiment, the phenothiazine is perphenazine. In a particular embodiment, the drug combination comprises phenazopyridine and dacarbazine. In another particular embodiment, the drug combination comprises phenazopyridine and perphenazine. In another specific embodiment, the drug combination comprises phenazopyridine and phenelzine.

Aminopyridine Compounds

By “aminopyridine” is meant any pyridine ring-containing compound in which the pyridine has one, two, or three amino group substitutents. Other substitutents may optionally be present. Exemplary aminopyridines include, for example, phenazopyridine, 4-aminopyridine, 3,4-diaminopyridine, 2,5-diamino-4-methylpyridine, 2,3,6-triaminopyridine, 2,4,6-triaminopyridine, and 2,6-diaminopyridine, the structures of which are depicted in the table entitled “Exemplary Aminopyridine Compounds” herein.

Phenazopyridine

Phenazopyridine (PZP) is an exemplary aminopyridine. Other aminopyridines similar to phenazopyridine include 4-aminopyridine (E-1), 3,4-diaminopyridine (E-4), 2,5-diamino-4-methylpyridine (E-2), 2,3,6-triaminopyridine (E-5), 2,4,6-triaminopyridine (E-3), and 2,6-diaminopyridine (E-6), the structures of which are depicted below.

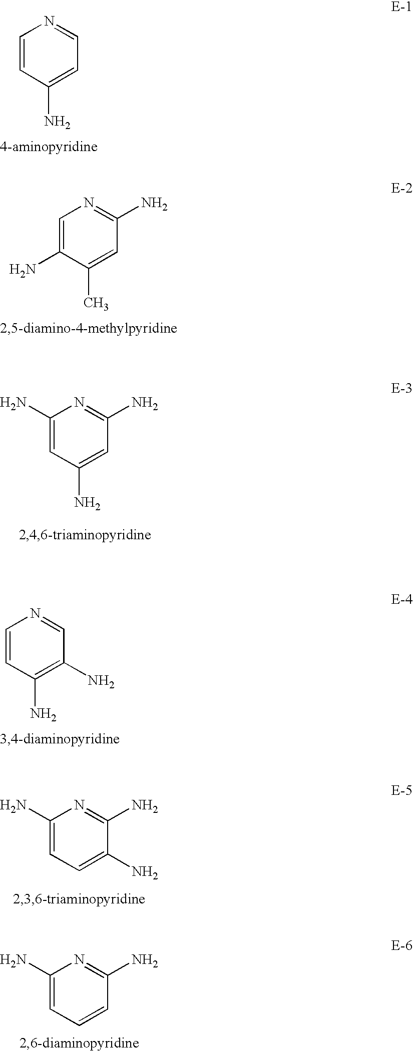

Phenazopyridine base (2,6-diamino-3-(phenylazo)pyridine) and its salt, phenazopyridine-HCl, are classified as medicinal azo dyes. The HCl salt form of phenazopyridine has the molecular formula C₁₁H₁₂ClN₅ with a molecular weight of 249.7. They are light to dark red to dark violet crystalline powders, near odorless, and slightly soluble in water and alcohol. Pharmaceutical phenazopyridine is usually synthesized as an HCl salt and prepared in tablet form. Phenazopyridine is usually prescribed to treat dysuria and urinary tract infections (UTI), acting as a local analgesic, and is not in itself a xenobiotic. Phenazopyridine is often prescribed in combination with sulphonamide compounds for treating UTIs. The structure of phenazopyridine —HCl is: 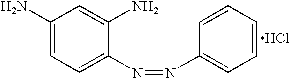
Phenazopyridine and Aminopyridine Analogs

Aminopyridines can accommodate many modifications while still maintaining structural and therapeutic efficacy. Phenazopyridine and derivatives thereof have been disclosed in U.S. Pat. Nos. 1,680,108; 1,680,109; 1,680,110; and 1,680,111. Modification of the medicinal azo dyes, di-amino(phenylazo)pyridines have been performed to improve solubility in water by reacting these compounds with alkylating agents (e.g., alkyl halides and alkyl sulphates) to produce quaternary pyridinium bases (see, e.g., U.S. Pat. No. 2,135,293). Heterocyclic azo derivatives and N-substituted diaminopyridines have also been described (U.S. Pat. No. 2,145,579 and U.S. Pat. No. 3,647,808, hereby incorporated by reference).

Phenazopyridine Metabolites

Phenazopyridine metabolites have been previously described in the literature (e.g., Thomas et al., J. Pharm. Sci. 79:321-325, 1990 and Jurima-Romet et al., Biopharm. Drug Disp. 14:171-179, 1992; hereby incorporated by reference). In humans, the major urinary phenazopyridine metabolite is the hydroxylation product of the pyridine ring, 2,6-diamino-5-hydroxy-3-(phenylazo)pyridine (5-OH-phenazopyridine). Other minor hydroxylated phenazopyridine metabolites include 2,6-diamino-5,4′-dihydroxy-3-(phenylazo)pyridine, 2,6-diamino-4′-hydroxy-3-(phenylazo)pyridine, and 2,6-diamino-2′-hydroxy-3-(phenylazo)pyridine. Cleavage of the azo bond results in the formation of a tri-aminopyridine and an aniline. The tri-aminopyridine metabolites can subsequently be further metabolized to mono, di, or other tri-aminopyridines and the aniline to aminophenols respectively.

Phenothiazines

Phenothiazines that are useful in the antimicrobial combination of the invention are compounds having the general formula (XIV): 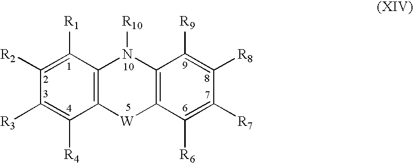
wherein R₂ is selected from the group consisting of: 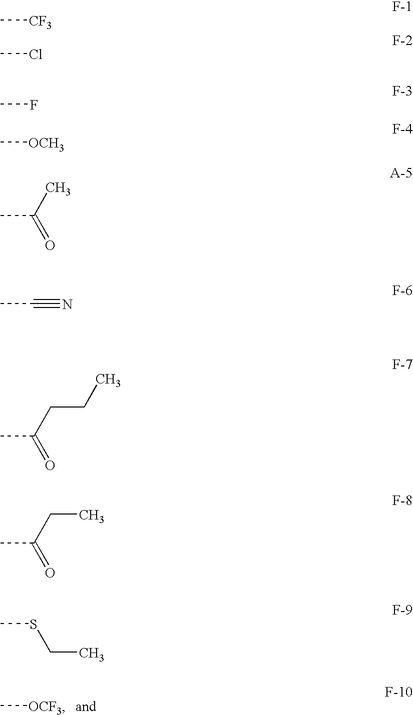
wherein each of R₁, R₃, R₄, R₅, R₆, R₇, R₈, and R₉ is, independently, H, OH, F, OCF₃, or OCH₃; and wherein W is selected from the group consisting of: 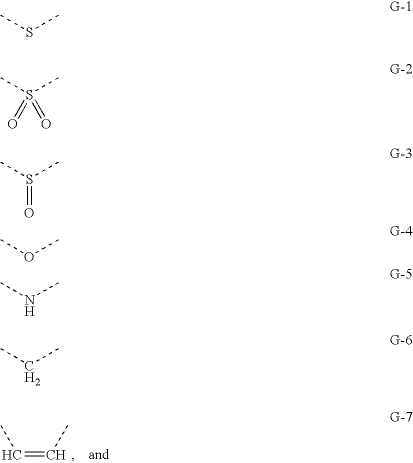
wherein R₁₀ is selected from the group consisting of:

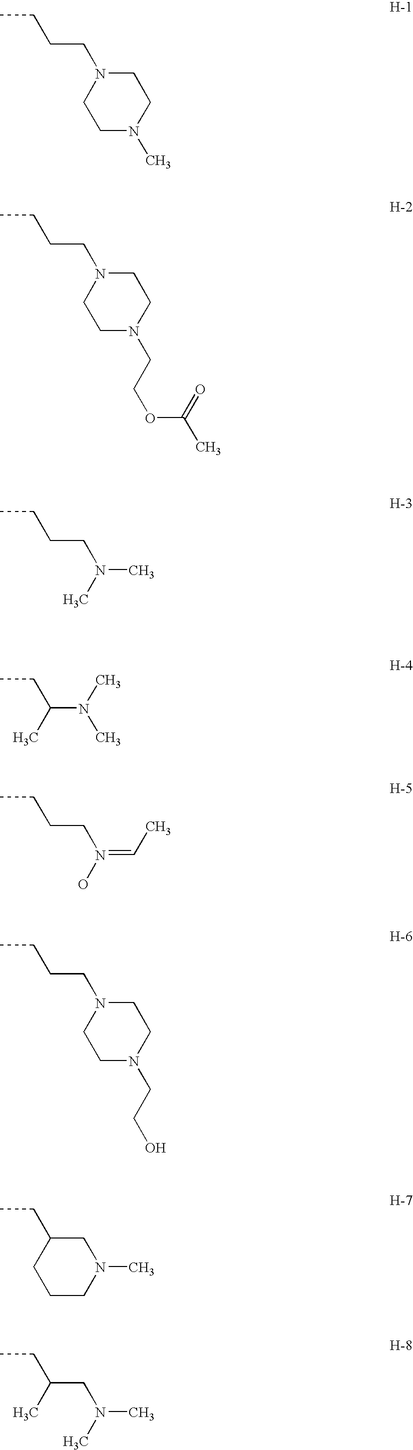

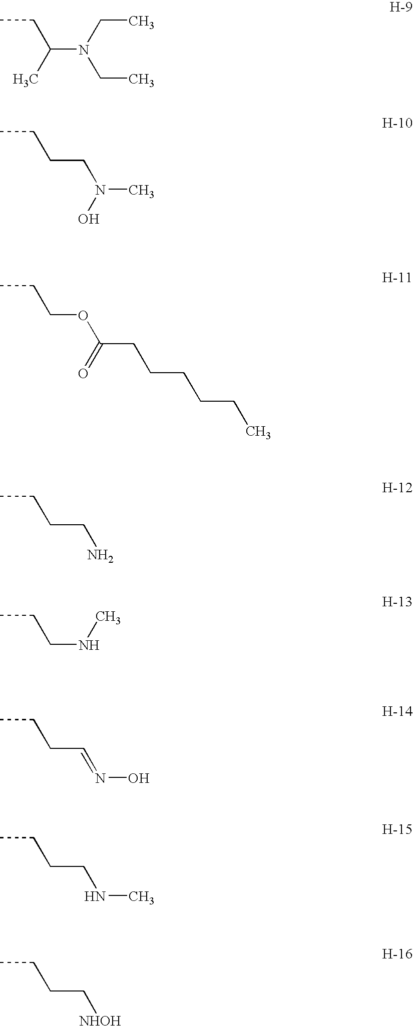

In certain embodiments of the compounds, R₂ is Cl; each of R₁, R₃, R₄, R₅, R₆, R₇, R₈, and R₉ is H or F. In other certain embodiments, each of R₁, R₄, R₅, R₆, and R₉ is H.

A commonly prescribed member of the phenothiazine family is perphenazine, which has the following formula:

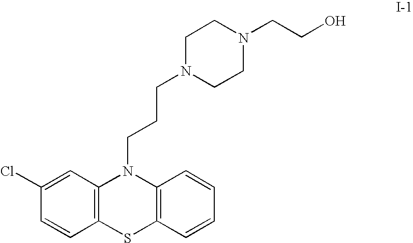

Perphenazine is currently formulated for oral and systemic administration. Perphenazine is a white-light yellow crystal or crystalline powder and is easily soluble in methanol, ethanol, and chloroform. It is slightly soluble in ether and shows relative insolubility in water. It is chemically designated 4-[3-(2-chlorophenothiazin-10-yl)propyl]-1-piperazineethanol and has a molecular formula of C21H26ClN3OS with a molecular weight of 403.97.

Phenothiazines undergo extensive metabolic transformation into a number of metabolites that may be therapeutically active. These metabolites may be substituted for phenothiazines in the antimicrobial combinations of the invention. The metabolism of perphenazine yields, for example, oxidative N-demethylation to yield the corresponding primary and secondary amine, aromatic oxidation to yield a phenol, N-oxidation to yield the N-oxide, S-oxidation to yield the sulphoxide or sulphone, oxidative deamination of the aminopropyl side chain to yield the phenothiazine nuclei, and glucuronidation of the phenolic hydroxy groups and tertiary amino group to yield a quaternary ammonium glucuronide.

Dacarbazine, an antineoplastic agent, is a synthetic analog of a purine precursor and is used for the treatment of metastatic melanoma and Hodgkin's lymphoma. Dacarbazine is colorless to ivory colored crystalline and is poorly soluble in water and ethanol. Dacarbazine is poorly absorbed from the GI tract and is most commonly administered as an i.v. injection or infusion. Following i.v. injection, dacarbazine is metabolized, mostly in the liver, to its active form, as a monomethyl triazino derivative—the same active metabolite seen in an analog of dacarbazine, temozolomide.

Phenelzine, a hydrazine, is a yellowish-white powder that is highly soluble in water and very poorly soluble in alcohol.

Drug Combination Comprising a Quaternary Ammonium Compound and an Anti-Fungal Imidazole, Haloprogin Manganese Sulfate, or Zinc Chloride

In certain embodiments, the drug combination that has anti-scarring activity comprises at least two agents, wherein at least one agent is a quaternary ammonium compound and at least one second agent is selected from an anti-fungal imidazole, haloprogin, manganese sulfate (MnSO₄) and zinc chloride (ZnCl₂). In a particular embodiment, the quaternary ammonium compound includes pentolinium, hexamethonium, pentamethonium, tetramethylammonium, tetraethylammonium, trimethaphan, trimethidium, and chlorisondamine. In a particular embodiment, the quaternary ammonium compound is pentolinium. In another particular embodiment, an anti-fungal imidazole compound includes ketoconazole, sulconazole, clotrimazole, econazole, miconazole, oxiconazole, tioconazole, and butoconazole. In a specific embodiment, the anti-fungal imidazole compound is ketoconazole or sulconazole. In a specific embodiment, the drug combination comprises pentolinium and haloprogin; in another specific embodiment, the drug combination comprises pentolinium and manganese sulfate; in yet another specific embodiment, the drug combination comprises pentolinium and zinc chloride; and in another specific embodiment, the drug combination comprises pentolinium and sulconazole.

Quaternary Ammonium Compounds

Quaternary ammonium compounds are those in which the nitrogen atom has four group substitutents. Quaternary ammonium compounds may be mono-, symmetrical bisquaternary, or asymmetrical bisquaternary compounds. Exemplary quaternary ammonium compounds are pentolinium (L-1), hexamethonium (L-3), pentamethonium (L-5), tetramethylammonium (L-4), tetraethylammonium (L-2), trimethidium (L-7), and chlorisondamine (L-6), the structures of which are depicted below.

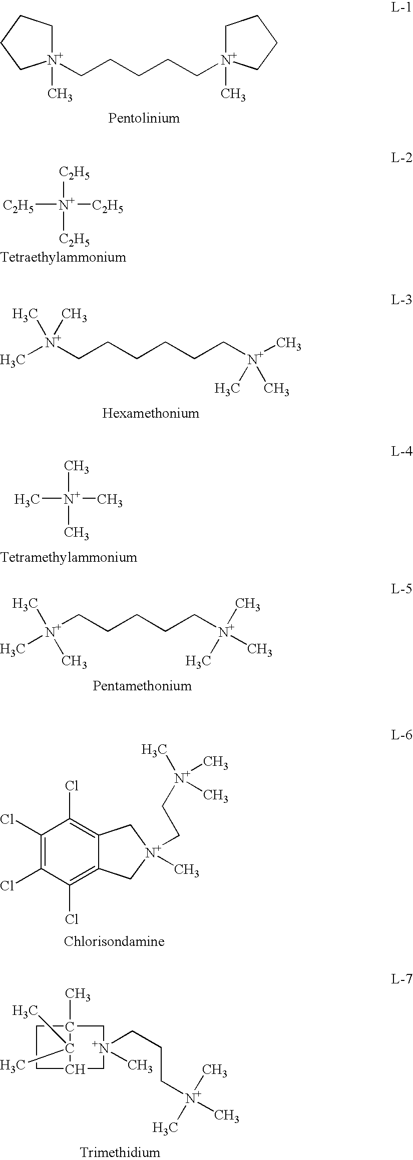

Pentolinium (pentamethylene-1,5-bis(N-methylpyrrolidinium) and its salt, pentolinium ditartrate, are symmetrical bisquaternary ammonium compounds. The tartrate salt form of pentolinium has the molecular formula C₂₃H₄₂N₂O₁₂ with a molecular weight of 538.6. Pentolinium ditartrate is a white powder, near odorless, and highly soluble in water.

The aforementioned quaternary ammonium compounds exhibit peripheral ganglionic blocking activity and have been used in anesthesia for controlled hypotension. The structure of pentolinium ditartrate (M-1) is: 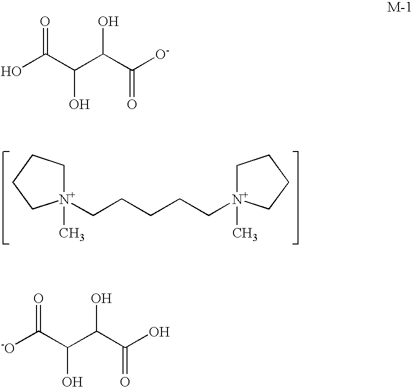
Pentolinium Analogs

Quaternary ammonium compounds can accommodate many modifications while still maintaining structural and therapeutic efficacy. Pentolinium and its derivatives are described in U.S. Pat. No. 4,902,720 and U.S. Pat. No. 6,096,788, each of which is hereby incorporated by reference. Any of the quaternary ammonium analogs described in these patents can be used in a drug combination described herein.

Haloprogin is a halogenated phenolic ether having the chemical formula C₉H₄C₁₃IO. This drug is used in the treatment of surface fungal infections, for example, tinea pedis (athlete's foot), tinea cruris, tinea corporis, and tinea manuum.

Drug Combination Comprising an Antiestrogen and a Phenothiazine, Cupric Chloride, Dacarbazine, Methoxsalen, or Phenylezine

In certain embodiments, the drug combination that has anti-scarring activity comprises at least two agents, wherein at least one agent is an antiestrogen compound and at least one second agent is selected from phenothiazine, cupric chloride, dacarbazine, methoxsalen, and phenelzine. In specific embodiments, antiestrogens include tamoxifen, 4-hydroxy tamoxifen, clomifene, raloxifene, and faslodex. In certain specific embodiments, the antiestrogen is tamoxifen. In certain embodiments, a phenothiazine is selected from perphenazine, chlorpromazine, prochlorperazine, mepazine, methotrimeprazine, acepromazine, thiopropazate, perazine, propiomazine, putaperazine, thiethylperazine, methopromazine, chlorfenethazine, cyamemazine, enanthate, trifluoperazine, thioridazine, and norchlorpromazine. In a particular embodiment, the phenothiazine is perphenazine. In a specific embodiment, the drug combination comprises tamoxifen and cupric chloride; in another specific embodiment, the drug combination comprises tamoxifen and dacarbazine; in still another specific embodiment, the drug combination comprises tamoxifen and methoxsalen; in another specific embodiment, the drug combination comprises tamoxifen and perphenazine; and in still another specific embodiment, the drug combination comprises tamoxifen and phenelzine.

As described herein exemplary antiestrogenic compounds are tamoxifen (K-1), 4-hydroxy tamoxifen (K-4), clomifene (K-2), raloxifene (K-5), and faslodex (ICI 182,780; K-3), the structures of which, are depicted above. Phenothiazines, for example, compounds having the structure of formula (XIV), derivatives, and metabolites thereof are described in greater detail herein. Dacarbazine as described herein exhibits antineoplastic activity and is used for the treatment of metastatic melanoma and Hodgkin's lymphoma. Dacarbazine is colorless to ivory colored crystalline and is poorly soluble in water and ethanol. Following intravenous injection, dacarbazine is metabolized, mostly in the liver, to its active form, as a monomethyl triazino derivative—the same active metabolite seen in an analog of dacarbazine, temozolomide.

Methoxsalen is a white to cream colored, odorless crystal, which is very poorly soluble in water, slightly soluble in alcohol, and readily soluble in propylene glycol. This drug is well absorbed in the GI tract and is available as a composition that may be used in oral and topical forms. Methoxsalen is rapidly demethylated to 8-hydroxypsoralen and can subsequently conjugated with glucuronic acid and sulphate.

Certain compounds used in the drug combinations described herein include disulfuram, methoxsalen, phenelzine, ribavirin, estradiol, dacarbazine, haloprogin, and temozolomide, the structures of which are illustrated below. All of the compounds described here are each separately known in the art; see e.g., Goodman & Gilman's The Pharmacological Basis of Therapeutics, Tenth Edition (J. G. Hardman, L. E. Limbird, A. G. Gilman, eds.), McGraw-Hill, New York, 2001; and hereby incorporated by reference. 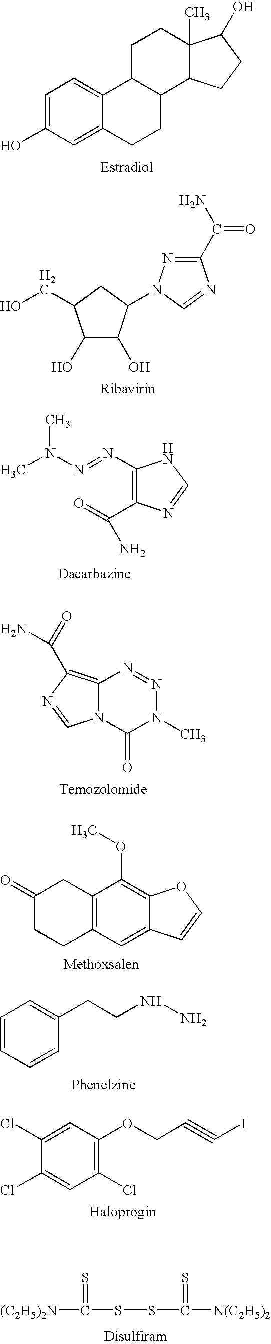
Drug Combination Comprising an Anti-Fungal Imidazole and Disulfuram or Ribavirin

In certain embodiments, the drug combination that has anti-scarring activity comprises at least two agents, wherein at least one agent is an anti-fungal imidazole compound and at least one second agent is either disulfuram or ribavirin. In certain specific embodiments, anti-fungal imidazole compounds include ketoconazole, sulconazole, clotrimazole, econazole, miconazole, oxiconazole, tioconazole, and butoconazole. In a particular embodiment, the anti-fungal imidazole compound is ketoconazole or sulconazole. Each of the compounds in this drug combination have been described in detail herein. In a specific embodiment, the drug combination comprises ketoconazole and disulfuram; in another specific embodiment, the drug combination comprises ketoconazole and ribavirin.

Drug Combination Comprising an Estrogen and Dacarbazine

In certain embodiments, the drug combination that has anti-scarring activity comprises at least two agents, wherein at least one agent is an estrogen compound and at least one second agent is dacarbazine. In specific embodiments, estrogenic compounds include estradiol, estradiol valerate, estradiol cypionate, ethinyl estradiol, estriol, mestranol, quinestrol, estrone, estrone sulfate, equilin, diethylstilbestrol, and genistein. In a particular embodiment, the estrogenic compound is estradiol, or a salt of estradiol. In a specific embodiment, the drug combination comprises estradiol and dacarbazine. Dacarbazine is described herein.

As used herein, an “estrogenic compound” means any compound that has an activity of estrogen. These activities include binding to the estrogen receptors ERα and ERβ, and promoting the effects of such binding, including DNA-binding, dimerization, and transcriptional activation of target genes. Estrogenic compounds can be naturally-occurring (e.g., estradiol, estron, and estriol) or synthetic, non-steroidal compounds (e.g., diethylstilbesterol and genistein). Dacarbazine is described herein.

As described herein compounds useful in the drug combinations include those described herein in any of their pharmaceutically acceptable forms, including isomers such as diastereomers and enantiomers, salts, solvates, and polymorphs thereof, as well as racemic mixtures and pure isomers of the compounds described herein.

Drug Combination Comprising an Amphotericin Compound and a Dithiocarbamoyl Disulfide Compound

In certain embodiments, the drug combination that has anti-scarring activity comprises at least two agents, wherein at least one agent is an anti-fungal drug, such as an amphotericin, particularly amphotericin B, and at least one second agent is a dithiocarbamoyl disulfide compound, such as disulfuram. On the basis of similar activity among different anti-fungal agents, amphotericin can be replaced by a different anti-fungal agent in the combination. Likewise, on the basis of similar activity among different dithiocarbamoyl disulfide family members, disulfuram can be replaced by a different dithiocarbamoyl disulfide in the combination.

In certain specific embodiments, the anti-fungal agent is chosen from amphotericin B, amorolfine, anidulafungin, butenafine, butoconazole, candidin, carbol-fuchsin, caspofungin, ciclopirox, clotrimazole, dapsone, econazole, enilconazole, fluconazole, flucytosine, gentian violet, griseofulvin, haloprogin, itraconazole, ketoconazole, mafenide, micafungin, miconazole, naftifine, nystatin, oxiconazole, pimaricin, posaconazole, ravoconazole, rimocidin, silver sulfadiazine, sulconazole, terbinafine, terconazole, tioconazole, tolnaftate, undecylenic acid, vacidin A, and voriconazole, while the compound of formula (XV), (XVI), or (XVII) (as described herein) is chosen from: disulfuram (bis(diethylthiocarbamoyl) disulfide), bis(dimethylthiocarbamoyl) disulfide, bis(dipropylthiocarbamoyl) disulfide, bis(dibutylthiocarbamoyl) disulfide, bis(dipentylthiocarbamoyl) disulfide, bis(di(2-methylpropyl)thiocarbamoyl) disulfide, bis(piperidinothiocarbamoyl) disulfide, bis(morpholinothiocarbamoyl) disulfide, bis((4-methylpiperazino)thiocarbamoyl) disulfide, bis((4-(2-hydroxyethyl)piperazino)thiocarbamoyl) disulfide, bis((hexahydro-4-methyl-1H-1,4-diazepin-1-yl)thiocarbamoyl) disulfide, and bis(3,3-dimethylcarbazoyl) disulfide.

The combination of an anti-fungal drug, such as amphotericin B, and a dithiocarbamoyl disulfide, such as disulfuram, has anti-fungal activity greater than that of either amphotericin B or disulfuram alone. Thus, combinations of disulfuram and amphotericin B may also be useful for the treatment of fungal infections. In addition, the using these two agents in combination has potential to mitigate side effects that could be encountered by using amphotericin B alone at high doses.

By “anti-fungal agent” is meant an agent that reduces or inhibits the growth of a fungus by at least 10%, relative to an untreated control, with the proviso that the agent does not belong to the dithiocarbamoyl disulfide class of compounds. Exemplary anti-fungal agents are provided herein.

Amphotericin B

Amphotericin B is a polyene antibiotic isolated from Streptomyces nodosus. It contains a macrolide ring and an aminosugar, mycosamine. The formula of amphotericin B is:

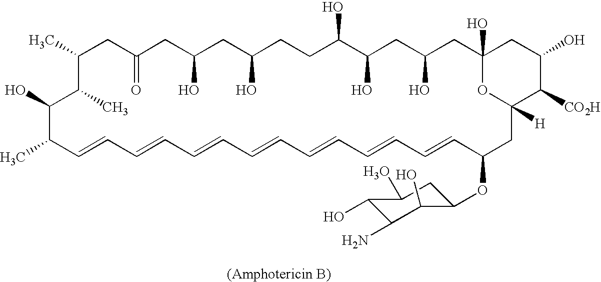

Amphotericin B is currently used for a wide range of systemic fungal infections and is formulated for IV injection and administered in this manner or intrathecally. Amphotericin B is poorly water soluble, but is sufficiently soluble that it is administered by IV infusion (0.1 mg/mL) or (0.3 mg/mL) in 5% dextrose. Amphotericin B is unstable in solution, particularly in normal saline. Other polyene macrolide anti-fungal agents include nystatin, candidin, rimocidin, vacidin A, and pimaricin.

Other Anti-Fungal Agents

Anti-fungal agents are known that derive their mechanism of action by their inhibition of cytochrome-P450 activity, which decreases conversion of 14-alpha-methylsterols to ergosterol. Failure of ergosterol synthesis causes altered membrane permeability leading to loss of ability to maintain a normal intracellular environment. Examples of anti-fungal agents that inhibit ergosterol biosynthesis by their cytochrome-P450 activity are fluconazole, itraconazole, ketoconazole, clotrimazole, butoconazole, econazole, ravuconazole, oxiconazole, posaconazole, sulconazole, terconazole, tioconazole, and voriconazole. Other anti-fungal agents that are egosterol biosynthesis inhibitors act by blocking squalene epoxidation. Examples of anti-fungal agents that inhibit ergosterol biosynthesis by blocking squalene epoxidation are amorolfine, butenafine, naftifine, and terbinafine.

Flucytosine is an anti-fungal agent that is known to derive its mechanism of action by its antimetabolic activity. It is converted to 5-fluorouracil (5-FU), which inhibits thymidylate synthetase and thereby inhibits fungal protein synthesis.

Griseofulvin is an anti-fungal agent that inhibits fungal mitosis by disrupting the mitotic spindle through its interaction with polymerized microtubules.

Anti-fungal agents are also known that serve as glucan synthesis inhibitors. Glucan is a key component of the fungal cell wall, and inhibition of this enzyme produces significant anti-fungal effects. Examples of glucan synthesis inhibitors are caspofungin, micafungin, and anidulafungin.

Disulfuram, or another dithiocarbamoyl disulfide, may be used in combination with any of the foregoing anti-fungal agents such that the dose of the anti-fungal agent is lowered and any side effects resulting from its mechanism of action mitigated.

Dithiocarbamoyl Disulfides

Disulfuram [bis(diethylthiocarbamoyl)disulfide] is a member of the dithiocarbamoyl disulfide class of compounds. It occurs as a white to off-white, odorless, and almost tasteless powder, soluble in water to the extent of about 20 mg/100 mL, and in alcohol to the extent of about 3.8 mg/100 mL. It is currently formulated for oral administration, with each tablet containing 250 mg or 500 mg of disulfuram. Its formula is:

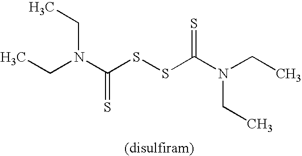

Some analogs of disulfuram have the following formulae:

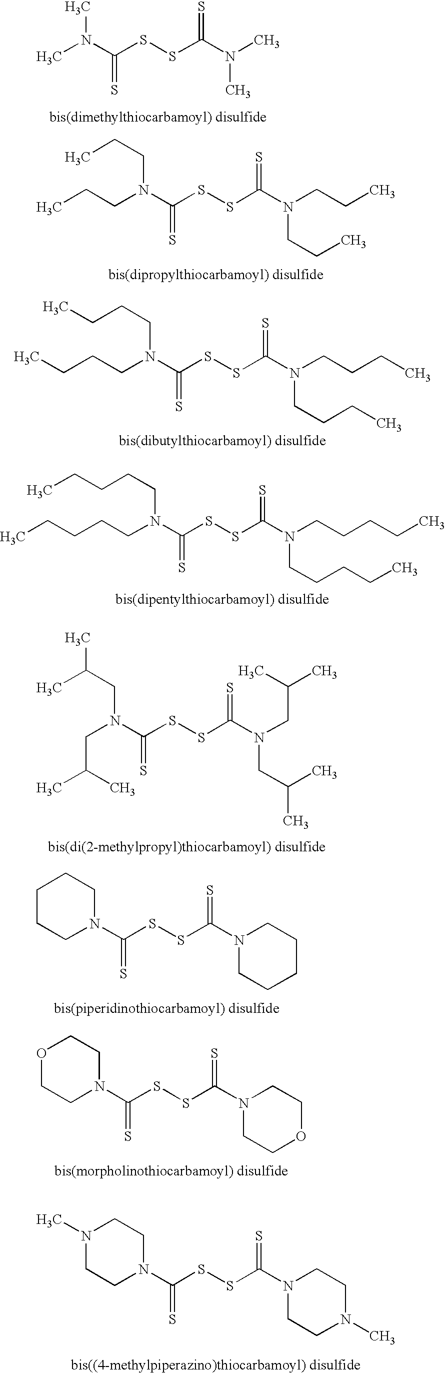

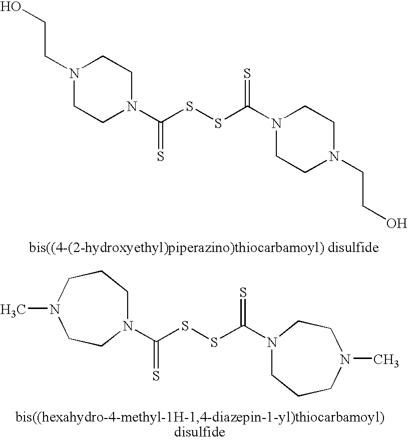

Dithiocarbamoyl disulfide compounds also include analogs that have structures of the following formulas (XV), (XVI), and (XVII): 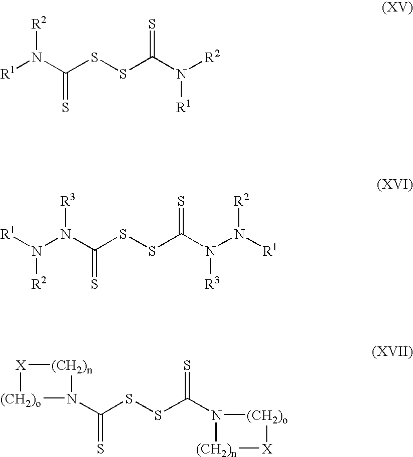
wherein X is CH₂, O, S, NR⁴, N(CH₂)ₚOR⁵, CH(CH₂)_{q}OR⁶, CH(CH₂)ᵣCO₂R⁷, CH(CH₂)ₛCONR⁸R⁹, 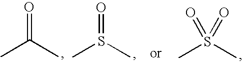
where R¹ and R² are independently C₁-C₈ linear or branched alkyl, alkaryl, or aryl, R³, R⁴, R⁵, R⁶, R⁷, and R⁹ are independently H, C₁-C₈ linear or branched alkyl, alkaryl, or aryl, n is 0-3, o is 2-4, p is 2-6, and q, r, or s is 0-6.

By “aromatic residue” is meant an aromatic group having a ring system with conjugated π electrons (e.g., phenyl, or imidazole). The ring of the aryl group preferably has 5 to 10 atoms. The aromatic ring may be exclusively composed of carbon atoms or may be composed of a mixture of carbon atoms and heteroatoms (i.e., nitrogen, oxygen, sulfur, and phosphorous). Aryl groups may optionally include monocyclic, bicyclic, or tricyclic rings, where each ring has preferably five or six members. The aryl group may be substituted or unsubstituted. Exemplary substitutents include alkyl, hydroxyl, alkoxy, aryloxy, sulfhydryl, alkylthio, arylthio, halo, fluoroalkyl, carboxyl, carboxyalkyl, amino, aminoalkyl, monosubstituted amino, disubstituted amino, and quaternary amino groups.

The term “aryl” means mono or bicyclic aromatic or heteroaromatic rings or ring systems. Examples of aryl groups include phenyl, naphthyl, pyrrolyl, furanyl, indolyl, benzofuranyl, benzothiophenyl, imidazolyl, triazolyl, tetrazolyl, benzimidazolyl, oxazolyl, benzoxazolyl, thiazolyl, benzothiazolyl, pyrazolyl, benzopyrazolyl, isoxazolyl, benzisoxazolyl, isothiazolyl, benzisothiazolyl, pyridinyl, quinolinyl, and isoquinolinyl.

“Heterocyclyl” means non-aromatic rings or ring systems that contain at least one ring hetero atom (e.g., O, S, N, P). Heterocyclic groups include, for example, pyrrolidinyl, tetrahydrofuranyl, morpholinyl, thiazolidinyl, and imidazolidinyl groups.

Aryl and heterocyclyl groups may be unsubstituted or substituted by one or more substitutents selected from the group consisting of C₁₋₁₀ alkyl, hydroxy, halo, nitro, C₁₋₁₀ alkoxy, C₁₋₁₀ alkylthio, trihalomethyl, C₁₋₁₀ acyl, arylcarbonyl, heteroarylcarbonyl, nitrile, C₁₋₁₀ alkoxycarbonyl, oxo, arylalkyl (wherein the alkyl group has from 1 to 10 carbon atoms) and heteroarylalkyl (wherein the alkyl group has from 1 to 10 carbon atoms).

Compounds useful in the drug combinations described herein include those described herein in any of their pharmaceutically acceptable forms, including racemic mixtures and substantially pure isomers (e.g., diastereomers, enantiomers) of compounds described herein, as well as salts, solvates, and polymorphs thereof.

Pharmaceutically acceptable salts of disulfuram and related dithiocarbamoyl disulfides are also useful compounds of the invention, as are metal chelates of these compounds. Preferred metals include, for example, copper, manganese, iron, and zinc.

Drug Combination Comprising an Anti-Fungal Compound and a Manganese Compound

In certain embodiments, the drug combination that has anti-scarring activity comprises at least two agents, wherein at least one agent is an anti-fungal drug, such as an allylamine, and at least one second agent is a manganese compound. In a specific embodiment, the allylamine compound is terbinafine. In certain embodiments, the manganese compound is manganese sulfate or manganese chloride. In a specific embodiment, the drug combination comprises terbinafine and manganese sulfate. In certain embodiments, the anti-fungal agent is selected from terbinafine, N-(5,5-dimethylhex-3-yn-1-yl)-N-methyl-1-naphthalenemethanamine, (E)-N-(6,6-dimethyl-2-hepten-4-ynyl)-N-(iminomethyl)-1-naphthalenemethanamine, (E)-N-(6,6-dimethyl-2-hepten-4-ynyl)-N-(1-iminoethyl)-1-naphthalenemethanamine, (Z)-N-(3-chloro-6,6-dimethyl-2-hepten-4-ynyl)-N-methyl-1-naphthalenemethanamine, and N-methyl-N-propargyl-2-aminotetralin. In another embodiment, the anti-fungal agent is selected from fluconazole, itraconazole, ketoconazole, posaconazole, ravuconazole, voriconazole, clotrimazole, econazole, miconazole, oxiconazole, sulconazole, terconazole, and tioconazole. In a certain particular embodiment, the anti-fungal agent is haloprogin. In certain embodiments, the drug combination further comprises an antibacterial agent selected from tetracyclines, macrolides, lincosamides, ketolides, fluoroquinolones, glycopeptide antibiotics, and polymyxin antibiotics or analog thereof. In a certain embodiment, the antibacterial agent is selected from gentamicin, amikacin, kanamycin, framycetin, neomycin, netilmicin, streptomycin, and tobramycin. In another embodiment, the antibacterial agent is selected from silver sulfadiazine, sodium sulfacetamide, sulfamethoxazole, sulfanilamide sulfasalazine, sulfisoxazole, trimethoprim, sulfamethoxazole, and triple sulfa.

Terbinafine is a synthetic anti-fungal agent that inhibits ergosterol biosynthesis via inhibition of squalene epoxidase, an enzyme part of the fungal sterol synthesis pathway that creates the sterols needed for the fungal cell membrane. In vitro, terbinafine has activity against most Candida spp., Aspergillus spp., Sporothrix schenckii, Penicillium marneffei, Malassezia furfur, Cryptococcus neoformans, Trichosporon spp. and Blastoschizomyces.

In addition to terbinafine, allylamines include amorolfine, butenafine, naftifine, N-(5,5-dimethylhex-3-yn-1-yl)-N-methyl-1-naphthalenemethanamine, (E)-N-(6,6-dimethyl-2-hepten-4-ynyl)-N-(iminomethyl)-1-naphthalenemethanamine, (E)-N-(6,6-dimethyl-2-hepten-4-ynyl)-N-(1-iminoethyl)-1-naphthalenemethanamine, (Z)-N-(3-chloro-6,6-dimethyl-2-hepten-4-ynyl)-N-methyl-1-naphthalenemethanamine, and N-methyl-N-propargyl-2-aminotetralin, some of which are shown in the table 3 below.

TABLE 3

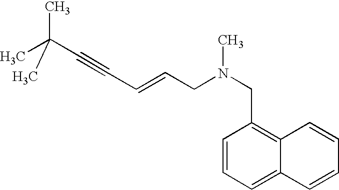

Terbinafine

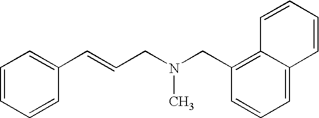

Naftifine

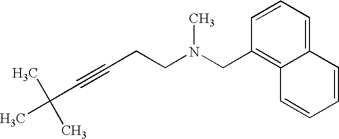

N-(5,5-Dimethylhex-3-yn-1-yl)-N-methyl-1-naphthalenemethanamine

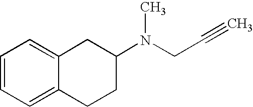

N-Methyl-N-propargyl-2-aminotetralin

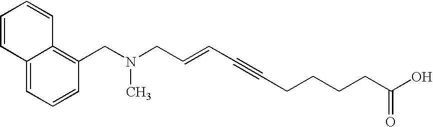

C₂₂H₂₅NO₂

Other allylamine or allylamine analogs that can be used in the methods, kits, and compositions of the invention are described in U.S. Pat. Nos. 4,202,894; 4,282,251; 4,751,245; 4,755,534; 5,021,458; 5,132,459; 5,234,946; 5,334,628; 5,935,998; and 6,075,056.

Other Anti-Fungal Agents

Other anti-fungal agents suitable for use in the drug combinations and related methods are described below. The anti-fungal azoles are preferred. Anti-fungal azoles are generally within in two classes, the imidizoles, such as miconazole, ketoconazole, and clotrimazole; and the triazoles, such as fluconazole, voriconazole, and ravuconazole. Other azoles are azaconazole, bromuconazole bitertanol, propiconazole, difenoconazole, diniconazole, cyproconazole, epoxiconazole, fluquinconazole, flusilazole, flutriafol, hexaconazole, itraconazole, imazalil, imibenconazole, ipconazole, tebuconazole, tetraconazole, fenbuconazole, metconazole, myclobutanil, perfurazoate, penconazole, posaconazole, pyrifenox, prochloraz, terconazole, triadimefon, triadimenol, triflumizole, and triticonazole.

Exemplary anti-fungal agents are selected from fluconazole, itraconazole, ketoconazole, posaconazole, ravuconazole, voriconazole, clotrimazole, econazole miconazole, oxiconazole, sulconazole, terconazole, tioconazole, nikkomycin Z, caspofungin, micafungin (FK463), anidulafungin (LY303366), amphotericin B (AmpB), AmpB lipid complex, AmpB colloidal dispersion, liposomal AmpB, liposomal nystatin, nystatin, pimaricin, lucensomycin, griseofulvin, ciclopirox olamine, haloprogin, tolnaftate, undecylenate, gentamicin, amikacin, kanamycin, framycetin, neomycin, netilmicin, streptomycin, tobramycin, silver sulfadiazine, sodium sulfacetamide, sulfamethoxazole, sulfanilamide sulfasalazine, sulfisoxazole, trimethoprim, sulfamethoxazole, triple sulfa, amrolfine, fenpropimorph, butenafine, and flucytosine.

Manganese Compounds

As used herein, a “manganese compound” is any salt or a complex of manganese. By “manganese salt” is meant any compound that results from replacement of part or all of the acid hydrogen of an acid by manganese. Manganese salts include, without limitation, acetate, adipate, alginate, ascorbate, aspartate, benzoate, bicarbonate, borate, butyrate, camphorate, carbonate, chlorate, clorite, citrate, cyanate, digluconate, fumarate, glucoheptanoate, glutamate, glycerophosphate, heptanoate, hexanoate, hydroxide, hypochlorite, lactate, maleate, nicotinate, nitrate, nitrite, oxalate, oxide, palmitate, pamoate, pectinate, perchlorate, peroxide, 3-phenylpropionate, phosphate, hydrogen phosphate, dihydrogen phosphate, phosphite, picrate, pivalate, propionate, salicylate, suberate, succinate, tartrate, triiodide, bromide, chloride, fluoride, and iodide. The salt can be the manganese salt of a metal complex, e.g. manganese(II) zinc bis(dithiocarbamate) (also known as Mancozeb). Preferred manganese salts are those of sulfur-containing anions including, without limitation, sulfide, sulphite, sulfate, bisulfate, bisulfite, persulfate, thiosulfate, hyposulfite, undecanoate sulfate, thiocyanate, benzenesulfonate, 2-hydroxyethanesulfonate, dodecylsulfate, hemisulfate, methanesulfonate, 2-naphthalenesulfonate, tosylate, ethanesulfonate, and camphorsulfonate. Desirably, the manganese compound is manganese sulfate or manganese chloride. Specifically excluded from the definition of “manganese compound” is manganese when present in food.

By “manganese complex” is meant a manganese compound including one or more chelate rings wherein the ring includes a manganese atom. Desirably, the complex is a macrocyclic or polydentate complexes of manganese. Manganese complexes include, without limitation, complexes of phenanthroline, 8-quinolinol, 2,6-diaminopyridine, bipyridine, diethylenetriamine, DPDP, EDDA, EDTA, EDTP, EDTA-BMA, DTPA, DOTA, DO3A, acetylacetonate, azamacrocycles, porphyrins, and Schiff-base complexes. Manganese complexes include those complexes described in U.S. Pat. Nos. 6,541,490, 6,525,041, 6,204,259, 6,177,419, 6,147,094, 6,084,093, 5,874,421, 5,637,578, 5,610,293, 5,246,847, 5,155,224, 4,994,259, 4,978,763, 4,935,518, 4,654,334, and 4,478,935. Binuclear, trinuclear, and tetranuclear complexes of manganese can also be used. Preferably, the manganese complex is a complex of ethylene-bis-dithiocarbamate. Most preferably, the manganese complex is manganese(II) ethylene bis(dithiocarbamate) (also known as Maneb). Methods for preparing manganese complexes are described in, for example, U.S. Pat. No. 5,155,224 and by F. A. Cotton and G. Wilkinson “Advanced Inorganic Chemistry,” John Wiley & Sons, 5^{th} Ed. (1988).

The manganese compounds described herein can be selected from any oxidation state (e.g., Mn(0) to Mn(VII)). In certain specific embodiments, the manganese compound is a manganous (e.g., Mn(II) compounds) or manganic (e.g., Mn(III)) salt or complex.

Additional Agents

When the manganese compound is incorporated as an enhancer in the formulation of an anti-fungal compound, it is desirable to include additional agents. The term “enhancer” as used herein refers to heightened or increased, especially, increased or improved quality or desirability of the combination of compounds. Thus, in some of the instances, the manganese compound may act as an enhancer of anti-fungal activity of a combination of anti-fungal agents. For example, when the manganese compound is used in combination with an allylamine-derived anti-fungal agent, such as terbinafine, or an azole-derived anti-fungal agent, such as fluconazole, itraconazole, or caspofungin, the manganese compound enhances the anti-fungal activity of these compounds against C. glabrata, thereby acting as an enhancer.

The additional agent administered may be any compound that is suitable for intravenous, rectal, oral, topical, intravaginal, ophthalmic, or inhalation administration. Preferably, such agents are administered to alleviate other symptoms of the disease or for co-morbid conditions. In general, this includes: antibacterial agents (e.g., sulfonamides, antibiotics, tetracyclines, aminoglycosides, macrolides, lincosamides, ketolides, fluoroquinolones, glycopeptide antibiotics, and polymyxin antibiotics); analgesic agents; antidiarrheals; antihelminthics; anti-infective agents such as antibiotics and antiviral agents; anti-fungal agents; antinauseants; antipruritics; antitubercular agents; antiulcer agents; antiviral agents; cough and cold preparations, including decongestants; diuretics; genetic materials; herbal remedies; nutritional agents, such as vitamins, essential amino acids and fatty acids; ophthalmic drugs such as antiglaucoma agents. Administration of the anti-fungal agent and manganese compound can be administered before, during, or after administration of one or more of the above agents.

For example, administration of a drug combination as described herein can be administered before, during, or after administration of one or more antibacterial agents. Exemplary antibacterial agents that can be administered include β-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenams (e.g., imipenem, ertapenem, and meropenem), and monobactams (e.g., astreonam); β-lactamase inhibitors (e.g., clavulanate, sulbactam, and tazobactam); tetracyclines (e.g., tetracycline, chlortetracycline, demeclocycline, minocycline, oxytetracycline, methacycline, and doxycycline); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); metronidazole; daptomycin; garenoxacin; ramoplanin; faropenem; polymyxin; tigecycline, AZD2563; and trimethoprim. These antibacterial agents can be used in the dose ranges currently known and used for these agents. Different concentrations may be employed depending, e.g., on the clinical condition of the patient, the goal of therapy (treatment or prophylaxis), the anticipated duration, and the severity of the infection for which the drug is being administered. Additional considerations in dose selection include the type of infection, age of the patient (e.g., pediatric, adult, or geriatric), general health, and comorbidity. Determining what concentrations to employ are within the skills of the pharmacist, medicinal chemist, or medical practitioner. Typical dosages and frequencies are provided, e.g., in the Merck Manual of Diagnosis & Therapy (17th Ed. M H Beers et al., Merck & Co.).

A drug combination described herein can also be administered along with an antiprotozoal agent, such as pentamidine, propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, or 2,4-bis(4-amidinophenyl)thiophene-bis-O-methylamidoxime.

Chelating agents can also be used with an anti-fungal agent and a manganese compound in the methods, compositions, and kits of the invention. Chelating agents include phosphonic acids, methylenglycine diacetic acid, iminodisuccinate, glutamate, N,N-bis(carboxymethyl, S,S′-ethylenediamine disuccinic acid (EDDS), β-alaninediacetic acid, ethylenediamine-N,N,N′,N′,-tetraacetic acid, ethylenediamine-N,N,N′,N′,-tetraacetic acid, disodium salt, dihydrate, ethylenediamine-N,N,N′,N′,-tetraacetic acid, trisodium salt, trihydrate, ethylenediamine-N,N,N′,N′-tetraacetic acid, tetrasodium salt, tetrahydrate, ethylenediamine-N,N,N′,N′-tetraacetic acid, dipotassium salt, dihydrate, ethylenediamine-N,N,N′,N′-tetraacetic acid, dilithium salt, monohydrate, ethylenediamine-N,N,N′,N′-tetraacetic acid, diammonium salt, ethylenediamine-N,N,N′,N′-tetraacetic acid, tripotassium salt, dihydrate, ethylenediamine-N,N,N′,N′-tetraacetic acid, ethylenediamine-N,N,N′,N′-tetraacetic acid, calcium chelate, ethylenediamine-N,N,N′,N′-tetraacetic acid, cerium chelate, ethylenediamine-N,N,N′,N′-tetraacetic acid, ethylenediamine-N,N,N′,N′-tetraacetic acid, ethylenediamine-N,N,N′,N′-tetraacetic acid, dysprosium chelate, ethylenediamine-N,N,N′,N′-tetraacetic acid, europium chelate, ethylenediamine-N,N,N′,N′-tetraacetic acid, iron chelate, ethylenediamine-N,N,N′,N′-tetraacetic acid, ethylenediamine-N,N,N′,N′-tetraacetic acid, ethylenediamine-N,N,N′,N′-tetraacetic acid, ethylenediamine-N,N,N′,N′-tetraacetic acid, ethylenediamine-N,N,N′,N′-tetraacetic acid, ethylenediamine-N,N,N′,N′-tetraacetic acid, samarium chelate, ethylenediamine-N,N,N′,N′-tetraacetic acid, ethylenediamine-N,N,N′,N′-tetraacetic acid, zinc chelate, trans-1,2-diaminocyclohexane-N,N,N′,N′-tetraaceticacid, monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid dihydrochloride, ethylenediamine-N,N′-bis(methylenephosphonic acid), hemihydrate, N-(2-hydroxyethyl)ethylenediamine-N,N,N′,N′-triacetic acid, ethylenediamine-N,N,N′,N′-tetrakis(methylenephosphonic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, barium chelate, cobalt chelate, copper chelate, indium chelate, lanthanum chelate, magnesium chelate, nickel chelate, strontium chelate, nitrilotripropionic acid, dimercaprol(2,3-dimercapto-1-propanol), nitrilotris(methylenephosphoric acid), trisodium salt, 7,19,30-trioxa-1,4,10,13,16,22,27,33-octaazabicyclo[11,11,11]pentatriacontane hexahydrobromide, and triethylenetetramine-N,N,N′,N″,N′″,N′″-hexaacetic acid. When the chelating agent is used in combination with an anti-fungal agent and a manganese compound, there is desirably a decrease in the consumption of either the anti-fungal agent or the manganese compound, or both.

Compounds useful in the invention include those described herein in any of their pharmaceutically acceptable forms, including isomers such as diastereomers and enantiomers, salts, solvates, and polymorphs thereof, as well as racemic mixtures of the compounds described herein.

Combinations Comprising Ciclopirox and Antiproliferative Agents

In certain embodiments, the drug combinations according to the present invention may comprise ciclopirox (or its structural or functional analogs, salts or metabolites) and an antiproliferative agent.

Ciclopirox

Ciclopirox (6-cyclohexyl-1-hydroxy-4-methyl-2(1H)-pyridinone) is a synthetic anti-fungal agent having a broad spectrum of activity. It can be fungistatic and fungicidal against species including, for example, Candida albicans, Trichophyton spp., Epidermophyton spp., and Aspergillus spp. Antibacterial properties of ciclopirox have also been demonstrated against both Gram-positive and Gram-negative species (Abrams et al., Clin. Dermatol., 9: 471-477, 1992). Anti-inflammatory activity including the inhibition of prostaglandin and leukotriene synthesis in human polymorphonuclear cells has also been reported.

Ciclopirox Analogs

Structural and functional analogs (e.g., mimosine) can replace ciclopirox in the therapeutic combinations of this invention. Structural ciclopirox analogs may be 2-pyridinones of general structure: 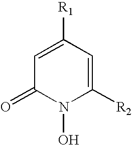
wherein R₁ is H, OH, NH₂, a halide, or any branched or unbranched, substituted or unsubstituted C₁₋₁₀ alkyl, C₁₋₁₀ alkoxyalkyl, C₁₋₁₀ hydroxyalkyl, C₁₋₁₀ aminoalkyl, C₁₋₁₀ alkylaminoalkyl, C₄₋₁₀ cycloalkyl, C₅₋₈ aryl, or C₆₋₂₀ alkylaryl, and R₂ is H, OH, NH₂, a halide, or any branched or unbranched, substituted or unsubstituted C₁₋₁₀ alkyl, C₁₋₁₀ alkoxyalkyl, C₁₋₁₀ hydroxyalkyl, C₁₋₁₀ aminoalkyl, C₁₋₁₀ alkylaminoalkyl, C₄₋₁₀ cycloalkyl, C₅₋₈ aryl, C₆₋₂₀ alkylaryl, C₃₋₁₀ heterocyclyl, or C₃₋₁₀ heteroaryl, wherein 1-4 carbon atoms of any of R₁ or R₂ may be substituted with one or more heteroatoms. Particularly useful R₁ groups include H, CH₃, CH₃CH₂, (CH₃)₂CH, (CH₃CH₂)₂CH, CH₃O, CH₃CH₂O, (CH₃)₂CHO, and (CH₃CH₂)₂CHO. Particularly useful R₂ groups include cyclopentyl, cyclohexyl, CH₂CH(CH₃)CH₂C(CH₃)₃, and 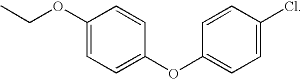
Particularly useful 2-pyridinones analogs, in addition to ciclopirox (R₁=CH₃; R₂=cyclohexyl), include octopirox (R₁=CH₃; R₂=CH₂CH(CH₃)CH₂C(CH₃)₃), and rilopirox (R₁=CH₃; R₂=

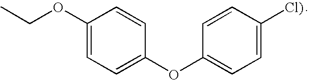

Methods for synthesizing 2-pyridinone derivatives are well known in the art (see, for example, U.S. Pat. Nos. 3,883,545 and 3,972,888).

Functional ciclopirox analogs, useful for combination therapy according to this invention, inhibit DNA initiation at origins of replication, are not purines or pyrimidines, and do not replace naturally occurring nucleotides during DNA synthesis. Functional ciclopirox analogs include, for example, mimosine and geminin. Inhibition of DNA initiation at origins of replication can be easily assessed using standard techniques. For example, replication intermediates isolated from cells cultured in the presence of the candidate ciclopirox analog can be assessed by 2D gel electrophoresis (Levenson et al., Nucleic Acid Res., 17: 3997-4004, 1993). This method takes advantage of the different electrophoretic properties of DNA fragments containing replication forks, replication bubbles, or termination structures. Fragments containing origins of replication are easily identified.

Antiproliferative Agents

“Antiproliferative agent” refers to a compound that, individually, inhibits the growth of a neoplasm. Antiproliferative agents include, but are not limited to microtubule inhibitors, topoisomerase inhibitors, platins, alkylating agents, and anti-metabolites.

By “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells.

Particular antiproliferative agents include paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine. Additional antiproliferative agents are listed in Table 4 below.

In certain embodiments, antiproliferative agents are paclitaxel, gemcitabine, doxorubicin, vinblastine, etoposide, 5-fluorouracil, or carboplatin.

TABLE 4
A

| | | |
|---|---|---|
| Alkylating agents | cyclophosphamide | lomustine |
| | busulfan | procarbazine |
| | ifosfamide | altretamine |
| | melphalan | estramustine phosphate |
| | hexamethylmelamine | mechlorethamine |
| | thiotepa | streptozocin |
| | chlorambucil | temozolomide |
| | dacarbazine | semustine. |
| | carmustine | |
| Platinum agents | cisplatin | carboplatinum |
| | oxaliplatin | ZD-0473 (AnorMED) |
| | spiroplatinum, | lobaplatin (Aeterna) |
| | carboxyphthalatoplatinum, | satraplatin (Johnson Matthey) |
| | tetraplatin | BBR-3464 (Hoffmann-La |
| | ormiplatin | Roche) |
| | iproplatin | SM-11355 (Sumitomo) |
| | | AP-5280 (Access) |
| Antimetabolites | azacytidine | tomudex |
| | gemcitabine | trimetrexate |
| | capecitabine | deoxycoformycin |
| | 5-fluorouracil | fludarabine |
| | floxuridine | pentostatin |
| | 2-chlorodeoxyadenosine | raltitrexed |
| | 6-mercaptopurine | hydroxyurea |
| | 6-thioguanine | decitabine (SuperGen) |
| | cytarabin | clofarabine (Bioenvision) |
| | 2-fluorodeoxy cytidine | irofulven (MGI Pharma) |
| | methotrexate | DMDC (Hoffmann-La Roche) |
| | idatrexate | ethynylcytidine (Taiho) |
| Topoisomerase inhibitors | amsacrine | rubitecan (SuperGen) |
| | epirubicin | exatecan mesylate (Daiichi) |
| | etoposide | quinamed (ChemGenex) |
| | teniposide or mitoxantrone | gimatecan (Sigma-Tau) |
| | irinotecan (CPT-11) | diflomotecan (Beaufour-Ipsen) |
| | 7-ethyl-10-hydroxy- | TAS-103 (Taiho) |
| | camptothecin | elsamitrucin (Spectrum) |
| | topotecan | J-107088 (Merck & Co) |
| | dexrazoxanet (TopoTarget) | BNP-1350 (BioNumerik) |
| | pixantrone (Novuspharma) | CKD-602 (Chong Kun Dang) |
| | rebeccamycin analogue | KW-2170 (Kyowa Hakko) |
| | (Exelixis) | |
| | BBR-3576 (Novuspharma) | |
| Antitumor antibiotics | dactinomycin (actinomycin D) | amonafide |
| | doxorubicin (adriamycin) | azonafide |
| | deoxyrubicin | anthrapyrazole |
| | valrubicin | oxantrazole |
| | daunorubicin (daunomycin) | losoxantrone |
| | epirubicin | bleomycin sulfate (blenoxane) |
| | therarubicin | bleomycinic acid |
| | idarubicin | bleomycin A |
| | rubidazone | bleomycin B |
| | plicamycinp | mitomycin C |
| | porfiromycin | MEN-10755 (Menarini) |
| | cyanomorpholinodoxorubicin | GPX-100 (Gem |
| | mitoxantrone (novantrone) | Pharmaceuticals) |
| Antimitotic agents | paclitaxel | SB 408075 (GlaxoSmithKline) |
| | docetaxel | E7010 (Abbott) |
| | colchicine | PG-TXL (Cell Therapeutics) |
| | vinblastine | IDN 5109 (Bayer) |
| | vincristine | A 105972 (Abbott) |
| | vinorelbine | A 204197 (Abbott) |
| | vindesine | LU 223651 (BASF) |
| | dolastatin 10 (NCI) | D 24851 (ASTAMedica) |
| | rhizoxin (Fujisawa) | ER-86526 (Eisai) |
| | mivobulin (Warner-Lambert) | combretastatin A4 (BMS) |
| | cemadotin (BASF) | isohomohalichondrin-B |
| | RPR 109881A (Aventis) | (PharmaMar) |
| | TXD 258 (Aventis) | ZD 6126 (AstraZeneca) |
| | epothulone B (Novartis) | PEG-paclitaxel (Enzon) |
| | T 900607 (Tularik) | AZ10992 (Asahi) |
| | T 138067 (Tularik) | IDN-5109 (Indena) |
| | cryptophycin 52 (Eli Lilly) | AVLB (Prescient |
| | vinflunine (Fabre) | NeuroPharma) |
| | auristatin PB (Teikoku | azaepothilone B (BMS) |
| | Hormone) | BNP-7787 (BioNumerik) |
| | BMS 247550 (BMS) | CA-4 prodrug (OXiGENE) |
| | BMS 184476 (BMS) | dolastatin-10 (NIH) |
| | BMS 188797 (BMS) | CA-4 (OXiGENE) |
| | taxoprexin (Protarga) | |
| Aromatase inhibitors | aminoglutethimide | exemestane |
| | letrozole | atamestane (BioMedicines) |
| | anastrazole | YM-511 (Yamanouchi) |
| | formestane | |
| Thymidylate synthase inhibitors | pemetrexed (Eli Lilly) | nolatrexed (Eximias) |
| | ZD-9331 (BTG) | CoFactor ™ (BioKeys) |
| DNA antagonists | trabectedin (PharmaMar) | mafosfamide (Baxter |
| | glufosfamide (Baxter | International) |
| | International) | apaziquone (Spectrum |
| | albumin + 32P (Isotope | Pharmaceuticals) |
| | Solutions) | O6 benzyl guanine (Paligent) |
| | thymectacin (NewBiotics) | |
| | edotreotide (Novartis) | |
| Farnesyltransferase | arglabin (NuOncology Labs) | tipifarnib (Johnson & Johnson) |
| inhibitors | lonafarnib (Schering-Plough) | perillyl alcohol (DOR |
| | BAY-43-9006 (Bayer) | BioPharma) |
| Pump inhibitors | CBT-1 (CBA Pharma) | zosuquidar trihydrochloride (Eli |
| | tariquidar (Xenova) | Lilly) |
| | MS-209 (Schering AG) | biricodar dicitrate (Vertex) |
| Histone acetyltransferase inhibitors | tacedinaline (Pfizer) | pivaloyloxymethyl butyrate |
| | SAHA (Aton Pharma) | (Titan) |
| | MS-275 (Schering AG) | depsipeptide (Fujisawa) |
| Metalloproteinase | Neovastat (Aeterna | CMT-3 (CollaGenex) |
| inhibitors | Laboratories) | BMS-275291 (Celltech) |
| | marimastat (British Biotech) | |
| Ribonucleoside reductase | gallium maltolate (Titan) | tezacitabine (Aventis) |
| | triapine (Vion) | didox (Molecules for Health) |
| inhibitors | | |
| TNF alpha agonists/antagonists | virulizin (Lorus Therapeutics) | revimid (Celgene) |
| | CDC-394 (Celgene) | |
| Endothelin A receptor antagonist | atrasentan (Abbott) | YM-598 (Yamanouchi) |
| | ZD-4054 (AstraZeneca) | |
| Retinoic acid receptor agonists | fenretinide (Johnson & | alitretinoin (Ligand) |
| | Johnson) | |
| | LGD-1550 (Ligand) | |
| Immuno-modulators | interferon | dexosome therapy (Anosys) |
| | oncophage (Antigenics) | pentrix (Australian Cancer |
| | GMK (Progenics) | Technology) |
| | adenocarcinoma vaccine | ISF-154 (Tragen) |
| | (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenics) |
| | synchrovax vaccines (CTL | β-alethine (Dovetail) |
| | Immuno) | CLL therapy (Vasogen) |
| | melanoma vaccine (CTL | |
| | Immuno) | |
| | p21 RAS vaccine (GemVax) | |
| Hormonal and antihormonal | estrogens | prednisone |
| agents | conjugated estrogens | methylprednisolone |
| | ethinyl estradiol | prednisolone |
| | chlortrianisen | aminoglutethimide |
| | idenestrol | leuprolide |
| | hydroxyprogesterone caproate | goserelin |
| | medroxyprogesterone | leuporelin |
| | testosterone | bicalutamide |
| | testosterone propionate; | flutamide |
| | fluoxymesterone | octreotide |
| | methyltestosterone | nilutamide |
| | diethylstilbestrol | mitotane |
| | megestrol | P-04 (Novogen) |
| | tamoxifen | 2-methoxyestradiol (EntreMed) |
| | toremofine | arzoxifene (Eli Lilly) |
| | dexamethasone | |
| Photodynamic agents | talaporfin (Light Sciences) | Pd-bacteriopheophorbide |
| | Theralux (Theratechnologies) | (Yeda) |
| | motexafin gadolinium | lutetium texaphyrin |
| | (Pharmacyclics) | (Pharmacyclics) |
| | | hypericin |
| Tyrosine Kinase Inhibitors | imatinib (Novartis) | kahalide F (PharmaMar) |
| | leflunomide (Sugen/Pharmacia) | CEP-701 (Cephalon) |
| | ZD1839 (AstraZeneca) | CEP-751 (Cephalon) |
| | erlotinib (Oncogene Science) | MLN518 (Millenium) |
| | canertinib (Pfizer) | PKC412 (Novartis) |
| | squalamine (Genaera) | phenoxodiol () |
| | SU5416 (Pharmacia) | trastuzumab (Genentech) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PMI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | EKB-569 (Wyeth) | |

B
Miscellaneous agents

| | |
|---|---|
| SR-27897 (CCK A inhibitor, Sanofi-Synthelabo) | BCX-1777 (PNP inhibitor, BioCryst) |
| tocladesine (cyclic AMP agonist, Ribapharm) | ranpirnase (ribonuclease stimulant, Alfacell) |
| alvocidib (CDK inhibitor, Aventis) | galarubicin (RNA synthesis inhibitor, Dong-A) |
| CV-247 (COX-2 inhibitor, Ivy Medical) | tirapazamine (reducing agent, SRI International) |
| P54 (COX-2 inhibitor, Phytopharm) | N-acetylcysteine (reducing agent, Zambon) |
| CapCell ™ (CYP450 stimulant, Bavarian Nordic) | R-flurbiprofen (NF-kappaB inhibitor, Encore) |
| GCS-100 (gal3 antagonist, GlycoGenesys) | 3CPA (NF-kappaB inhibitor, Active Biotech) |
| G17DT immunogen (gastrin inhibitor, Aphton) | seocalcitol (vitamin D receptor agonist, Leo) |
| efaproxiral (oxygenator, Allos Therapeutics) | 131-I-TM-601 (DNA antagonist, TransMolecular) |
| PI-88 (heparanase inhibitor, Progen) | eflornithine (ODC inhibitor, ILEX Oncology) |
| tesmilifene (histamine antagonist, YM BioSciences) | minodronic acid (osteoclast inhibitor, Yamanouchi) |
| histamine (histamine H2 receptor agonist, Maxim) | indisulam (p53 stimulant, Eisai) |
| tiazofurin (IMPDH inhibitor, Ribapharm) | aplidine (PPT inhibitor, PharmaMar) |
| cilengitide (integrin antagonist, Merck KGaA) | rituximab (CD20 antibody, Genentech) |
| SR-31747 (IL-1 antagonist, Sanofi-Synthelabo) | gemtuzumab (CD33 antibody, Wyeth Ayerst) |
| CCI-779 (mTOR kinase inhibitor, Wyeth) | PG2 (hematopoiesis enhancer, Pharmagenesis) |
| exisulind (PDE V inhibitor, Cell Pathways) | Immunol ™ (triclosan oral rinse, Endo) |
| CP-461 (PDE V inhibitor, Cell Pathways) | triacetyluridine (uridine prodrug, Wellstat) |
| AG-2037 (GART inhibitor, Pfizer) | SN-4071 (sarcoma agent, Signature BioScience) |
| WX-UK1 (plasminogen activator inhibitor, Wilex) | TransMID-107 ™ (immunotoxin, KS Biomedix) |
| PBI-1402 (PMN stimulant, ProMetic LifeSciences) | PCK-3145 (apoptosis promotor, Procyon) |
| bortezomib (proteasome inhibitor, Millennium) | doranidazole (apoptosis promotor, Pola) |
| SRL-172 (T cell stimulant, SR Pharma) | CHS-828 (cytotoxic agent, Leo) |
| TLK-286 (glutathione S transferase inhibitor, Telik) | trans-retinoic acid (differentiator, NIH) |
| PT-100 (growth factor agonist, Point Therapeutics) | MX6 (apoptosis promotor, MAXIA) |
| midostaurin (PKC inhibitor, Novartis) | apomine (apoptosis promotor, ILEX Oncology) |
| bryostatin-1 (PKC stimulant, GPC Biotech) | urocidin (apoptosis promotor, Bioniche) |
| CDA-II (apoptosis promotor, Everlife) | Ro-31-7453 (apoptosis promotor, La Roche) |
| SDX-101 (apoptosis promotor, Salmedix) | brostallicin (apoptosis promotor, Pharmacia) |
| ceflatonin (apoptosis promotor, ChemGenex) | |

Exemplary Drug Combinations

In certain other embodiments, the drug combinations comprise rilopirox and paclitaxel, rilopirox and gemcitabine, rilopirox and doxorubicin, rilopirox and vinblastine, rilopirox and etoposide, rilopirox and 5-fluorouracil, or rilopirox and carboplatin.

In certain other embodiments, the drug combinations comprise octopirox and paclitaxel, octopirox and gemcitabine, octopirox and doxorubicin, octopirox and vinblastine, octopirox and etoposide, octopirox and 5-fluorouracil, or octopirox and carboplatin.

In certain other embodiments, the drug combinations comprise mimosine and paclitaxel, mimosine and gemcitabine, mimosine and doxorubicin, mimosine and vinblastine, mimosine and etoposide, mimosine and 5-fluorouracil, or mimosine and carboplatin.

In certain other embodiments, the drug combinations comprise germinin and paclitaxel, germinin and gemcitabine, germinin and doxorubicin, germinin and vinblastine, germinin and etoposide, germinin and 5-fluorouracil, or germinin and carboplatin.

In certain embodiments, the drug combinations comprise ciclopirox and paclitaxel, ciclopirox and gemcitabine, ciclopirox and doxorubicin, ciclopirox and vinblastine, ciclopirox and etoposide, ciclopirox and 5-fluorouracil, or ciclopirox and carboplatin.

Combinations Comprising Niclosamide and Antiproliferative Agents

In certain embodiments, the drug combinations according to the present invention may comprise an antihelminthic agent (e.g., niclosamide or its structural or functional analogs, salts, or metabolites) and an antiproliferative agent.

Antihelminthic Agents

“Antihelminthic agent” refers to a compound that, individually, inhibits the growth of a parasitic worm. Desirably, growth rate is reduced by at least 20%, 30%, 50%, or even 70%. Examples of helminthes include cestodes, trematodes, nematodes, Fasciola, Schistosoma, planaria, filaria, and Trichinella.

Antihelminthic agents encompass a broad spectrum of modes of action which include: glutamate-gated chloride channel potentiating compounds such as ivermectin, abamectin, doramectin, moxidectin, niclofolan, and mylbemycin D; calcium permeability potentiators such as praziquantel; malate metabolism inhibitors such as diamphenethide; phosphoglycerate kinase and mutase inhibitors such as chlorsulon; and benzaniles (e.g., salicylanilide compounds).

Benzanilides

Benzanilides that can be used according to the methods of the invention include those that fit formula XVIII: 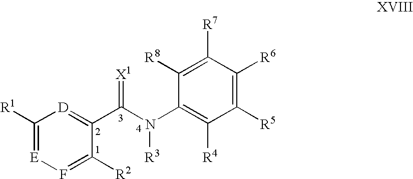
or a salt thereof. In formula XVIII, D is N or CR⁹; E is N or CR¹⁰; F is N or CR¹¹; and R¹ is H, halide, OR¹², SR¹³, NR¹⁴R¹⁵, or described by one of the formulas:

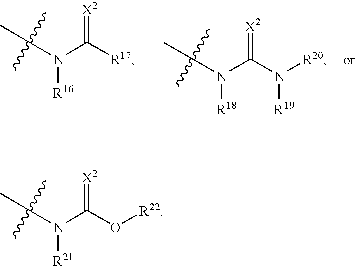

R² is H, OH, or OR¹²; R³ is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; or R² and R³ combine to form a six-membered ring in which position 1 is connected to position 4 by one of the groups:

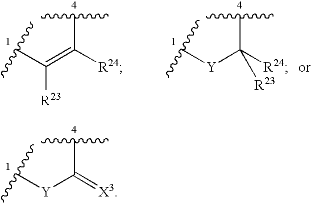

R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl; and R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, or are described by the formulas:

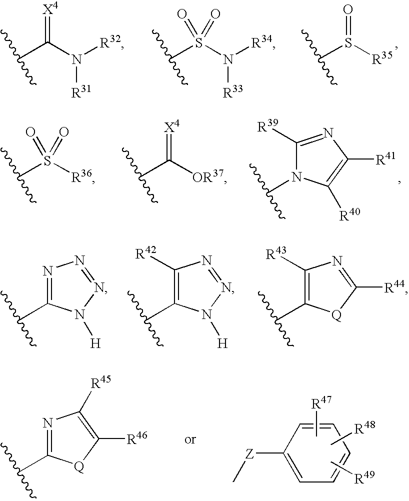

For compounds of formula XVIII, each X¹, X², X³, and X⁴ is, independently, O S; or NR³⁸; Y is CR²⁵R²⁶, O, S, or NR²⁷; Z is O, S, or CR⁵⁰R⁵¹; each Q is, independently, O, S, or NR⁵²; R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO₂; R¹² and R¹³ are each, independently, acyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁷, R²², R³⁵, R³⁶, R³⁷, R³⁸, and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴, and R⁴⁷ are each, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

In certain embodiments, X¹ is an oxygen atom; R² is OH; and R³ is H.

In certain other embodiments, X¹ is an oxygen atom; R² and R³ combine to form a six-membered ring in which position 1 is connected to position 4 by 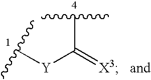
Y is an oxygen atom.

In certain other embodiments, X₁ is an oxygen atom; R² and R³ combine to form a six-membered ring in which position 1 is connected to position 4 by 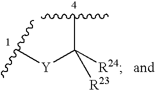
Y is an oxygen atom.

In certain embodiments, X¹ is an oxygen atom; R² is OH; D is CR⁹; E is CR¹⁰; F is CR¹¹; R¹ is halide; R¹¹ is hydrogen or halide; and R³, R⁹, and R¹⁰ are H.

Desirable compounds of formula XVIII are further described by any one of formulas XIX-XXII: 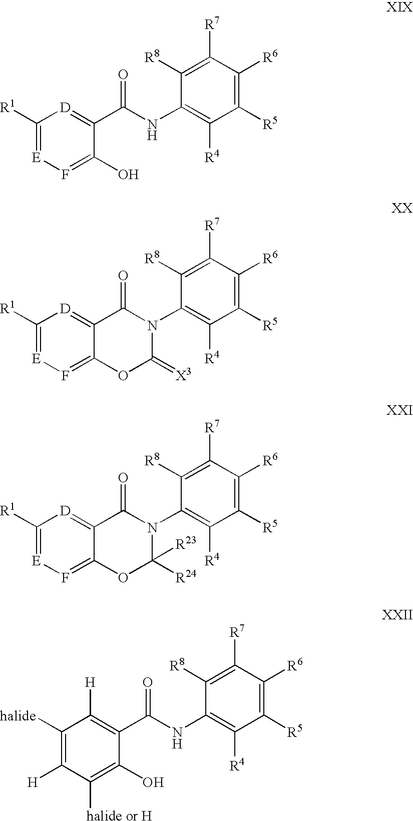
wherein F, E, D, X³, R¹, R⁴, R⁵, R⁶, R⁷, R⁸, R⁹, R¹⁰, R¹¹, R²³, and R²⁴ are as defined above.

Benzanilides that can be used according to the methods of the invention include various salicylanilides described in more detail below (e.g., niclosamide, oxyclozanide, closantel, resorantel, tribromsalan, clioxanide, dibromsalan, rafoxanide, flusalan), and the compounds disclosed in U.S. Pat. Nos. 3,041,236, 3,079,297, 3,113,067, 3,147,300, 3,332,996, 3,349,090, 3,449,420, 3,466,370, 3,469,006, 3,499,420, 3,798,258, 3,823,236, 3,839,443, 3,888,980, 3,906,023, 3,927,071, 3,949,075, 3,973,038, 4,005,218, 4,008,274, 4,072,753, 4,115,582, 4,159,342, 4,310,682, and 4,470,979, each of which is hereby incorporated by reference, Hlasta et al., Bioorg. Med. Chem., and European Patent No. 0533268. Salts or esters of any of these compounds can also be used according to the methods of the invention.

Salicylanilides

Salicylanilides consist of a salicylic acid ring and an anilide ring and are a subset of benzanilides. Exemplary salicylanilide compounds that can be used according to the present invention are depicted in Table 5.

TABLE 5

| | |
|---|---|
| | 4′-chloro-3-nitro- salicylanilide |
| 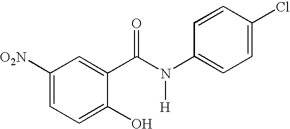 | 4′-chloro-5-nitro- salicyanilide |
| 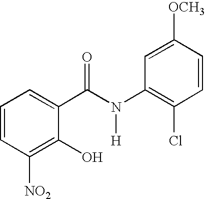 | 2′-chloro-5′-methoxy- 3-nitrosalicylanilide |
| 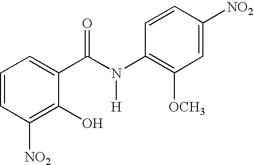 | 2′-methoxy-3,4′- dinitrosalicyanilide |
| 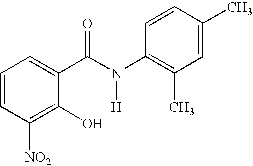 | 2′,4′-dimethyl-3- nitrosalicylanilide |
| 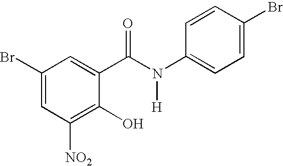 | 4′,5-dibromo-3-nitro- salicyanilide |
| 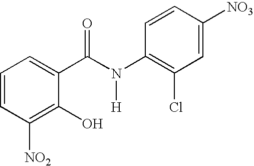 | 2′-chloro-3,4′-dinitro- salicyanilide |
| 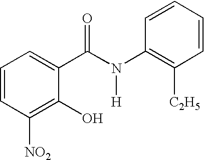 | 2′-ethyl-3-nitro- salicyanilide |
| 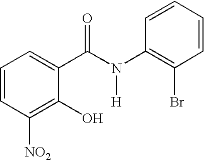 | 2′-bromo-3-nitro- salicyanilide |

Niclosamide

Niclosamide (2′,5-dichloro-4′-nitrosalicylanilide) is an antihelminthic used for treatment of cestode and trematode infestations in humans, pets, and, livestock. This drug has also been used as an effective lampricide and a pesticide against fresh water snails. The free base, the monohydrate, the ethanolamine salt, and the piperazine salt are know to be active as antihelmenthic agents. Niclosamide and its salts (e.g., the ethanolamine, piperazine, and monohydrate salts) exhibit very low toxicity in mammals. The structure of niclosamide and other benzanilide antihelmenthic agents are provided in Table 5.

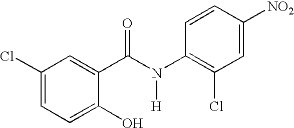

niclosamide

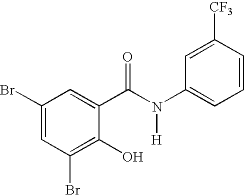

flusalan

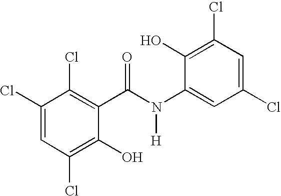

oxyclozanide

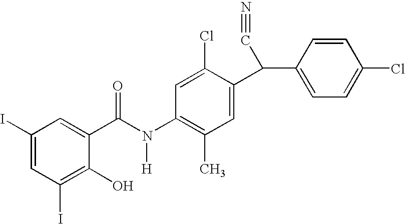

closantel

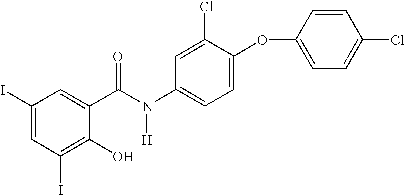

rafoxanide

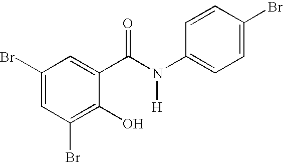

tribromsalan

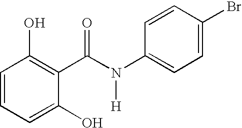

resoantel

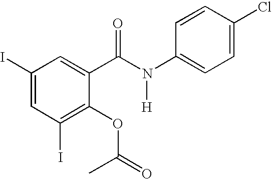

clioxanide

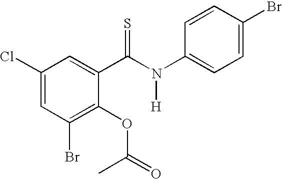

Brotianide

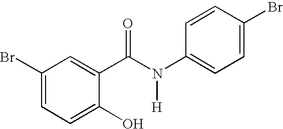

dibromsalan

Synthetic Methods

Methods for synthesizing benzanilide and salicylanilide derivatives are well known in the art. For example, niclosamide and related compounds can be prepared as described in U.S. Pat. Nos. 3,079,297 and 3,113,067; flusalan and related compounds can be prepared as described in U.S. Pat. No. 3,041,236; oxyclozanide and related compounds can be prepared as described in U.S. Pat. No. 3,349,090; closantel and related compounds can be prepared as described in U.S. Pat. No. 4,005,218; resorantel and related compounds can be prepared as described in U.S. Pat. No. 3,449,420; tribromsalan, dibromsalan, and related compounds can be prepared as described in U.S. Pat. Nos. 2,967,885 and 3,064,048; clioxanide and related compounds can be prepared as described by Campbell et al., Experientia 23:992 (1967); and rafoxanide and related compounds can be prepared as described by Mrozak et al., Experientia 25:883 (1969). Additional methods are disclosed by, for example, Hlasta et al., Bioorg. Med. Chem., U.S. Pat. Nos. 3,466,370, 3,888,980, 3,973,038, 4,008,274, 4,072,753, and 4,115,582, and European Patent No. 0533268. All publications and patents mentioned above are incorporated herein by reference.

Compounds of formula XXI can be prepared, for example, by condensation of a salicylanilide with an aldehyde, see reaction 1, as described in Acta Pharmaceutica (Zagreb) 50:239 (2000); or by reaction with acetylene, see reaction 2, as described in Khimiya Geterotsiklicheskikh Soedinenii 4:469 (1983) or Khimiya Geterotsiklicheskikh Soedinenii 9:1278 (1979).

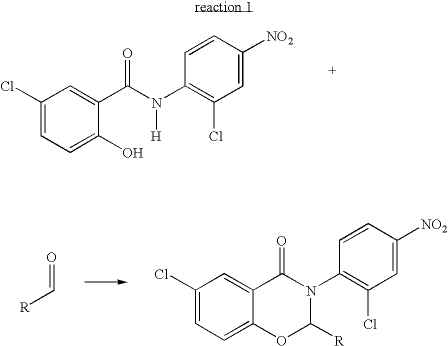

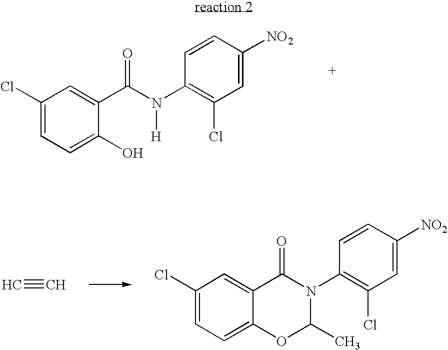

Compounds of formula XX in which X³ is an oxygen atom can be prepared, for example, by condensation of a salicylanilide with ethyl chloroformate, see reaction 3, as described in Pharmazie 45:34 (1990); J. Med. Chem. 32:807 (1989); or J. Med. Chem. 21:1178 (1978).

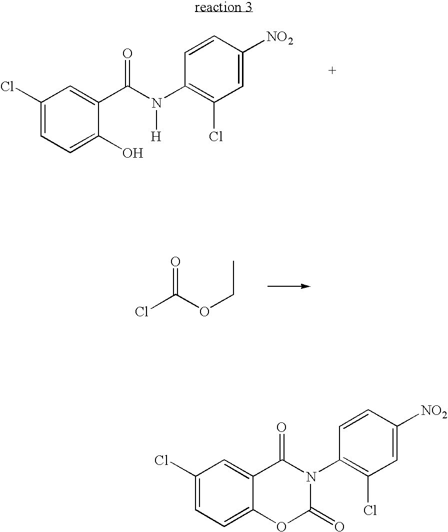

Compounds of formula XX in which X³ is a sulfur atom can be prepared, for example, by condensation of a salicylanilide with thiophosgene, see reaction 4, as described in Archiv der Pharmazie (Weinheim, Germany) 315:97 (1982); Indian J. Chem., Sect. B 18:352 (1979); Indian J. Chem., Sect. B 15:73 (1977); or Indian J. Pharm., 37:133 (1975).

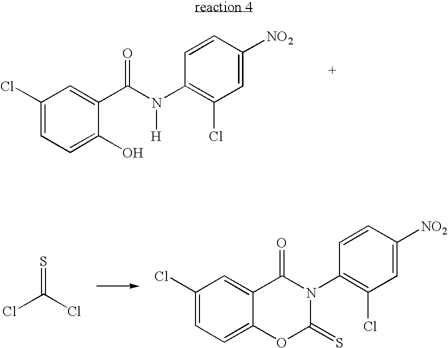

Compounds of formula XX in which X³ is NH can be prepared, for example, by reaction of a salicylanilide with cyanogen bromide, see reaction 5, as described in C. R. Hebd. Seances Acad. Sci., Ser. C 283:291 (1976).

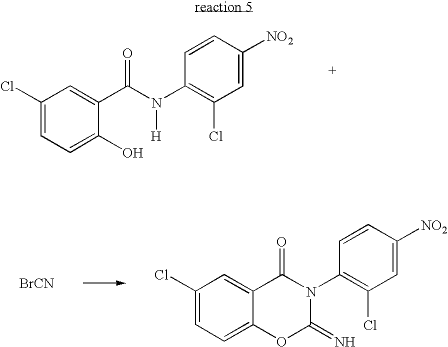

Compounds of formula XVIII in which D, E, or F is a nitrogen atom can be prepared using methods analogous to those used for the synthesis of salicylanilide compounds. For example, 2-hydroxynicotinic acid (Aldrich Cat. No. 25, 105-4), 3-hydroxypicolinic acid (Aldrich Cat. No. 15, 230-7), 6-hydroxynicotinic acid (Aldrich Cat. No. 12, 875-9), 6-hydroxypicolinic acid (Aldrich Cat. No. 38,430-5), 5-chloro-6-hydroxynicotinic acid (Fluka Cat. No. 24882), 5-bromonicotinic acid (Aldrich Cat. No. 22843-5), 2-chloronicotinic acid (Aldrich Cat. No. 15, 033-9), 6-chloronicotinic acid (Aldrich Cat. No. 15, 635-3), 5,6-dichloronicotinic acid (Aldrich Cat. No. 34, 021-9), or citrazinic acid (Aldrich Cat. No. 15, 328-1) can be reacted with an aniline to produce a compound of formula XVIII in which D, E, or F are a nitrogen atom. Furthermore, 2-hydroxynicotinic acid derivatives and 3-hydroxypyrazine-2-carboxylic acid derivatives can be prepared using the methods described in U.S. Pat. Nos. 5,364,940, 5,516,661, and 5,364,939. For example, 5-chloronicotinic acid (CAS 22620-27-5) can be hydroxylated using the methods described in U.S. Pat. No. 5,364,940 and the resulting 2-hydroxy-5-chloronicotinic acid coupled with 2-chloro-4-nitroaniline (Aldrich Cat. No. 45, 685-3), as shown in reaction 6, using standard amide coupling techniques.

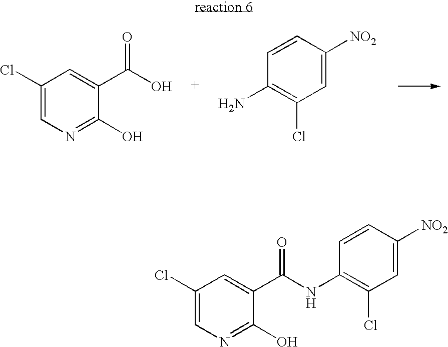

The resulting product is a compound of formula XVIII, and can be used in the methods of the invention.

Functional Analogs of Niclosamide

Based on the shared antihelmenthic activity, compounds such as ivermectin, abamectin, doramectin, moxidectin, mylbemycin D, niclofolan, praziquantel, diamphenethide, and chlorsulon can be substituted for niclosamide in the methods of the invention. Other antihelmenthic agents are known in the art; these compounds can also be employed in the methods of the invention.

Antiproliferative Agents

Antiproliferative agents that can be administered in the combinations of the invention are described above. Such agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors. Any one or more of the agents listed in Table 4 can be used. Exemplary antiproliferative agents include, without limitation, paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine.

Exemplary Drug Combinations

In certain embodiments, the drug combination comprises (1) an antihelminthic agent selected from the group consisting of niclosamide, oxyclozanide, closantel, rafoxanide, resorantel, clioxanide, tribromsalan, dibromsalan, brotianide, 4′-chloro-3-nitrosalicylanilide, 4′-chloro-5-nitrosalicylanilide, 2′-chloro-5′-methoxy-3-nitrosalicylanilide, 2′-methoxy-3,4′-dinitrosalicylanilide, 2′,4′-dimethyl-3-nitrosalicylanilide, 4′,5-dibromo-3-nitrosalicylanilide, 2′-chloro-3,4′-dinitrosalicylanilide, 2′-ethyl-3-nitrosalicylanilide, 2′-bromo-3-nitrosalicylanilide, flusalan, and a salt of the above listed agent and (2) an antiproliferative agent. In certain embodiments, the antiproliferative agent is selected from the group consisting of paclitaxel, gemcitabine, etoposide, irinotecan, and chlorpromazine.

In certain embodiments, the drug combination comprises (1) niclosamide or a salt or ester thereof and (2) an anti-proliferative agent. The niclosamide salt may be ethanolamine, piperazine, or monohydrate salt of niclosamide. In certain embodiments, the antiproliferative agent is selected from the group consisting of paclitaxel, gemcitabine, etoposide, irinotecan, and chlorpromazine.

In certain embodiments, the drug combination comprises (1) an antihelminthic agent selected from the group consisting of ivermectin, abamectin, doramectin, moxidectin, mylbemycin D, niclofolan, praziquantel, diamphenethide, and chlorsulon, and (2) an anti-proliferative agent. In certain embodiments, the antiproliferative agent is selected from the group consisting of paclitaxel, gemcitabine, etoposide, irinotecan, and chlorpromazine.

In other certain embodiments, the antihelminthic agent is selected from ivermectin, abamectin, doramectin, moxidectin, mylbemycin D, niclofolan, praziquantel, diamphenethide, and chlorsulon.

For example, in certain specific embodiments, the drug combination comprises niclosamide and paclitaxel, niclosamide and gemcitabine, niclosamide and etoposide, niclosamide and irinotecan, or niclosamide and chlorpromazine.

Combinations Comprising Chlorpromazine and Pentamidine

In certain embodiments, the drug combinations of the invention may comprise chlorpromazine (or its analogs, salts, or metabolites) and pentamidine (or its analogs, salts, or metabolites). In certain embodiments, the drug combination may further comprise one or more antiproliferative agents (e.g., those listed in Table 4).

Phenothiazines

Phenothiazines that are useful in the antiproliferative combination of the invention are compounds having the general formula (XXIII): 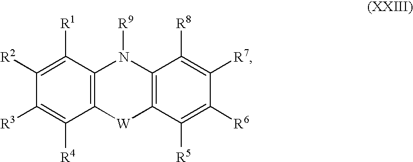
or a pharmaceutically acceptable salt thereof,

wherein R² is selected from the group consisting of: CF₃, halo, OCH₃, COCH₃, CN, OCF₃, COCH₂CH₃, CO(CH₂)₂CH₃, and SCH₂CH₃;

R⁹ has the formula: 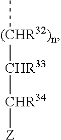
wherein n is 0 or 1, each of R³², R³³, and R³⁴ is, independently, H or substituted or unsubstituted C₁₋₆ alkyl, and Z is NR³⁵R³⁶ or OR³⁷, wherein each of R³⁵ and R³⁶ is, independently, H, substituted or unsubstituted C₁₋₆ alkyl, substituted or unsubstituted alkaryl, substituted or unsubstituted alkheteroaryl, and R³⁷ is H, C₁₋₆ alkyl, or C₁₋₇ acyl, wherein any of R³³, R³⁴, R³⁵, and R³⁶ can be optionally taken together with intervening carbon or non-vicinal O, S, or N atoms to form one or more five- to seven-membered rings, substituted with one or more hydrogens, substituted or unsubstituted C₁₋₆ alkyl groups, C₆₋₁₂ aryl groups, alkoxy groups, halogen groups, substituted or unsubstituted alkaryl groups, or substituted or unsubstituted alkheteroaryl groups;

each of R¹, R³, R⁴, R⁵, R⁶, R⁷, and R⁸ is independently H, OH, F, OCF₃, or OCH₃; and W is selected from the group consisting of:

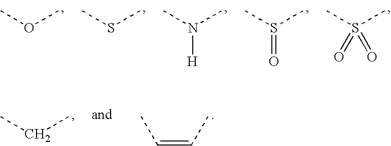

In certain embodiments, R⁹ is selected from the group consisting of:

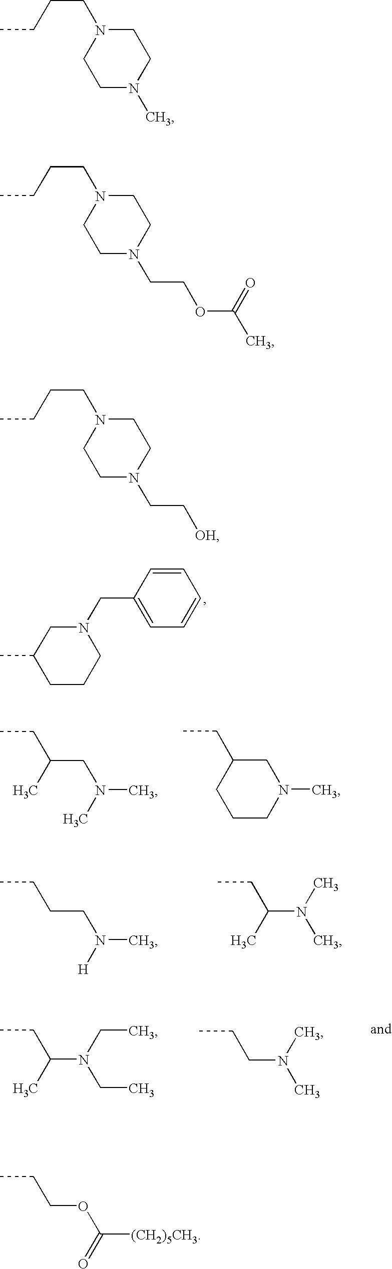

In certain embodiments, wherein R² is selected from the group consisting of: CF₃, halo, OCH₃, COCH₃, CN, OCF₃, COCH₂CH₃, CO(CH₂)CH₃, and SCH₂CH₃;

R⁹ is selected from the group consisting of: 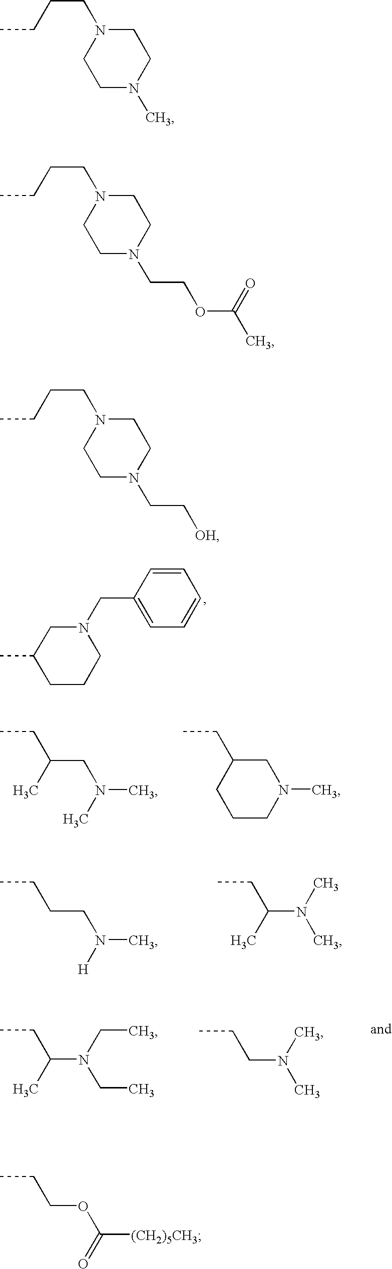
each of R¹, R³, R⁴, R⁵, R⁶, R⁷, and R⁸ is independently H, OH, F, OCF₃, or OCH₃; and W is selected from the group consisting of:

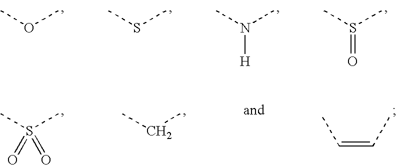

In certain embodiments, R₂ is Cl; each of R₁, R₃, R₄, R₅, R₆, R₇, R₈ is H or F; and R⁹ is selected from the group consisting of:

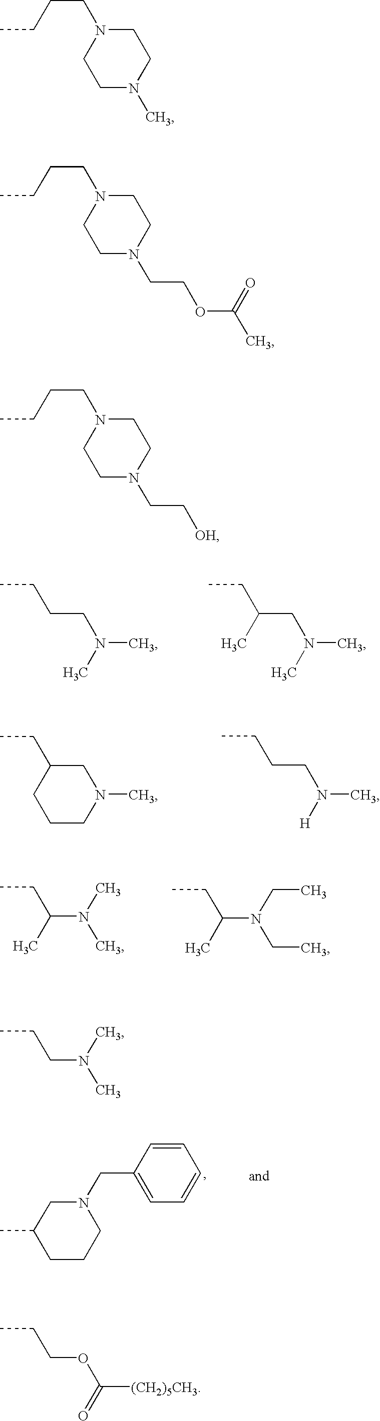

In certain embodiments, R₂, R₃, R₇ and R⁹ are as defined immediately above, and each of R₁, R₄, R₅, R₆, and R₈ is H.

In certain embodiments, the compound of formula (XXIII) is acepromazine, chlorfenethazine, cyamemazine, enanthate, fluphenazine, mepazine, methotrimeprazine, methoxypromazine, norchlorpromazine, perazine, perphenazine, prochlorperazine, promethazine, propiomazine, putaperazine, thiethylperazine, thiopropazate, thioridazine, trifluoperazine, or triflupromazine.

In certain other embodiments, the compound of formula (XXIII) is chlorpromazine, perphenazine or promethazine.

Chlorpromazine, Analogs and Metabolites

The most commonly prescribed member of the phenothiazine family is chlorpromazine, which has the structure:

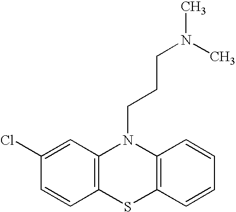

Chlorpromazine is currently available in the following forms: tablets, capsules, suppositories, oral concentrates and syrups, and formulations for injection.

Phenothiazines considered to be chlorpromazine analogs include fluphenazine, prochlorperazine, promethazine, thioridazine, and trifluoperazine. Many of these share antipsychotic or antiemetic activity with chlorpromazine. Also included as chlorpromazine analogs are those compounds in PCT Publication No. WO02/057244, which is hereby incorporated by reference.

Phenothiazines are thought to elicit their antipsychotic and antiemetic effects via interference with central dopaminergic pathways in the mesolimbic and medullary chemoreceptor trigger zone areas of the brain. Extrapyramidal side effects are a result of interactions with dopaminergic pathways in the basal ganglia. Although often termed dopamine blockers, the exact mechanism of dopaminergic interference responsible for the drugs' antipsychotic activity has not been determined.

Phenothiazines are also known to inhibit the activity of protein kinase C. Protein kinase C mediates the effects of a large number of hormones and is involved in may aspects of cellular regulation and carcinogenesis (Castagna, et al., J. Biol. Chem. 1982, 257:7847-51). The enzyme is also thought to play a role in certain types of resistance to cancer chemotherapeutic agents. Chlorpromazine has been investigated for the inhibition of protein kinase C both in vitro (Aftab, et al., Mol. Pharmacology, 1991, 40:798-805) and in vivo (Dwivedi, et al., J. Pharm. Exp. Ther., 1999, 291:688-704). Phenothiazines are also known as calmodulin inhibitors and mitotic kinesin inhibitors, the better of which modulate the movements of spindles and chromosomes in dividing cells.

Chlorpromazine also has strong alpha-adrenergic blocking activity and can cause orthostatic hypotension. Chlorpromazine also has moderate anticholinergic activity manifested as occasional dry mouth, blurred vision, urinary retention, and constipation. Chlorpromazine increases prolactin secretion owing to its dopamine receptor blocking action in the pituitary and hypothalamus.

Because chlorpromazine undergoes extensive metabolic transformation into a number of metabolites that may be therapeutically active, these metabolites may be substituted from chlorpromazine in the antiproliferative combination of the invention. The metabolism of chlorpromazine yields, for example, oxidative N-demethylation to yield the corresponding primary and secondary amine, aromatic oxidation to yield a phenol, N-oxidation to yield the N-oxide, S-oxidation to yield the sulphoxide or sulphone, oxidative deamination of the aminopropyl side chain to yield the phenothiazine nuclei, and glucuronidation of the phenolic hydroxy groups and tertiary amino group to yield a quaternary ammonium glucuronide.

In other examples of chlorpromazine metabolites useful in the antiproliferative combination of the invention, each of positions 3, 7, and 8 of the phenothiazine can independently be substituted with a hydroxyl or methoxyl moiety.

In certain embodiments, phenothiazines, or analogs, derivatives, or metabolites thereof, have a sedative activity.

Pentamidine, Analogs and Metabolites

Pentamidine

Pentamidine is currently used for the treatment of Pneumocystis carinii, Leishmania donovani, Trypanosoma brucei, T. gambiense, and T. rhodesiense infections. The structure of pentamidine is:

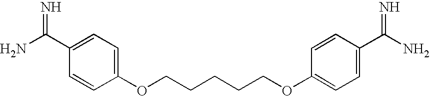

It is available formulated for injection or inhalation. For injection, pentamidine is packaged as a nonpyrogenic, lyophilized product. After reconstitution, it is administered by intramuscular or intravenous injection.

Pentamidine isethionate is a white, crystalline powder soluble in water and glycerin and insoluble in ether, acetone, and chloroform. It is chemically designated 4,4′-diamidino-diphenoxypentane di(β-hydroxyethanesulfonate). The molecular formula is C₂₃H₃₆N₄O₁₀S₂ and the molecular weight is 592.68.

The mode of action of pentamidine is not fully understood. In vitro studies with mammalian tissues and the protozoan Crithidia oncopelti indicate that the drug interferes with nuclear metabolism, producing inhibition of the synthesis of DNA, RNA, phospholipids, and proteins. Several lines of evidence suggest that the action of pentamidine against leishmaniasis, a tropical disease caused by a protozoan residing in host macrophages, might be mediated via host cellular targets and the host immune system. Pentamidine selectively targets intracellular leishmania in macrophages but not the free-living form of the protozoan and has reduced anti-leishmania activity in immunodeficient mice in comparison with its action in immunocompetent hosts.

Recently, pentamidine was shown to be an effective inhibitor of protein tyrosine phosphatase 1B (PTP1B). Because PTP1B dephosphorylates and inactivates Jak kinases, which mediate signaling of cytokines with leishmanicidal activity, its inhibition by pentamidine might result in augmentation of cytokine signaling and anti-leishmania effects. Pentamidine has also been shown to be a potent inhibitor of the oncogenic phosphatases of regenerating liver (such as, for example PRL-1, PRL-2, or PRL-3). Thus, in the methods of the invention, pentamidine can be replaced by any protein tyrosine phosphatase inhibitor, including PTP1B inhibitors or PRL inhibitors. Inhibitors of protein tyrosine phosphatases include levamisole, ketoconazole, bisperoxovanadium compounds (e.g., those described in Scrivens et al., Mol. Cancer Ther. 2:1053-1059, 2003, and U.S. Pat. No. 6,642,221), vandate salts and complexes (e.g., sodium orthovanadate), dephosphatin, dnacin A1, dnacin A2, STI-571, suramin, gallium nitrate, sodium stibogluconate, meglumine antimonate, 2-(2-mercaptoethanol)-3-methyl-1,4-naphthoquinone, 2,5-bis(4-amidinophenyl)furan-bis-O-methylamidoxime, known as DB289 (Immtech), 2,5-bis(4-amidinophenyl)furan (DB75, Immtech), disclosed in U.S. Pat. No. 5,843,980, and compounds described in Pestell et al., Oncogene 19:6607-6612, 2000, Lyon et al., Nat. Rev. Drug Discov. 1:961-976, 2002, Ducruet et al., Bioorg. Med. Chem. 8:1451-1466, 2000, U.S. Patent Application Publication Nos. 2003/0114703, 2003/0144338, 2003/0161893, and PCT Patent Publication Nos. WO99/46237, WO03/06788 and WO03/070158. Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, and U.S. Patent Application Publication Nos. US 2001/0044468 and US 2002/0019437, and the pentamidine analogs described in U.S. patent application Ser. No. 10/617,424 (see, e.g., Formula (II)). Other protein tyrosine phosphatase inhibitors can be identified, for example, using the methods described in Lazo et al. (Oncol. Res. 13:347-352, 2003), PCT Publication Nos. WO97/40379, WO03/003001, and WO03/035621, and U.S. Pat. Nos. 5,443,962 and 5,958,719.

Pentamidine has also been shown to inhibit the activity of endo-exonuclease (PCT Publication No. WO 01/35935). Thus, in the methods of the invention, pentamidine can be replaced by any endo-exonuclease inhibitor.

By “endo-exonuclease inhibitor” is meant a compound that inhibits (e.g., by at least 10%, 20%, 30%, or more) the enzymatic activity of an enzyme having endo-exonuclease activity. Such inhibitors include, but are not limited to, pentamidine, pentamidine analogs, and pentamidine metabolites.

By “phosphatase of regenerating liver inhibitor” is meant a compound that inhibits (e.g., by at least 10%, 20%, 30%, or more) the enzymatic activity of a member of the phosphatase of regenerating liver (PRL) family of tyrosine phosphatases. Members of this family include, but are not limited to, PRL-1, PRL-2, and PRL-3. Inhibitors include, but are not limited to, pentamidine, pentamidine analogs, and pentamidine metabolites.

By “protein tyrosine phosphatase 1B inhibitor” is meant a compound that inhibits (e.g., by at least 10%, 20%, 30%, or more) the enzymatic activity of protein phosphatase 1B. Inhibitors include, but are not limited to, pentamidine, pentamidine analogs, and pentamidine metabolites.

Pentamidine Analogs

Aromatic diamidino compounds can replace pentamidine in the antiproliferative combination of the invention. Aromatic diamidino compounds such as propamidine, butamidine, heptamidine, and nonamidine share properties with pentamidine in that they exhibit antipathogenic or DNA binding properties. Other analogs (e.g., stilbamidine and indole analogs of stilbamidine, hydroxystilbamidine, diminazene, benzamidine, 4,4′-(pentamethylenedioxy)phenamidine, dibrompropamidine, 1,3-bis(4-amidino-2-methoxyphenoxy)propane (DAMP), netropsin, distamycin, phenamidine, amicarbalide, bleomycin, actinomycin, and daunorubicin) also exhibit properties similar to those of pentamidine.

Pentamidine analogs are described, for example, by formula (XXIV) 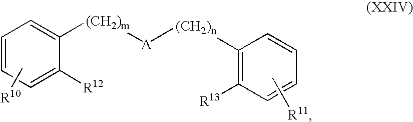
wherein A is 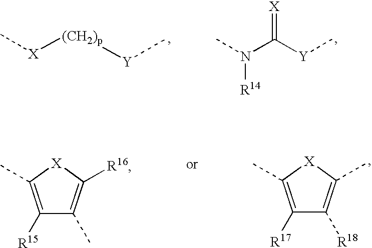
wherein

each of X and Y is, independently, O, NR¹⁹, or S,

each of R¹⁴ and R¹⁹ is, independently, H or C₁-C₆ alkyl,

each of R¹⁵, R¹⁶, R¹⁷, and R¹⁸ is, independently, H, C₁-C₆ alkyl, halogen, C₁-C₆ alkyloxy, C₆-C₁₈ aryloxy, or C₆-C₁₈ aryl-C₁-C₆ alkyloxy,

p is an integer between 2 and 6, inclusive,

each of m and n is, independently, an integer between 0 and 2, inclusive,

each of R¹⁰ and R¹¹ is 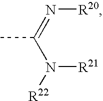
wherein

R²¹ is H, C₁-C₆ alkyl, C₁-C₈ cycloalkyl, C₁-C₆ alkyloxy-C₁-C₆ alkyl, hydroxy C₁-C₆ alkyl, C₁-C₆ alkylamino C₁-C₆ alkyl, amino C₁-C₆ alkyl, or C₆-C₁₈ aryl, R²² is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R²⁰ is H, OH, or C₁-C₆ alkyloxy, or R²⁰ and R²¹ together represent 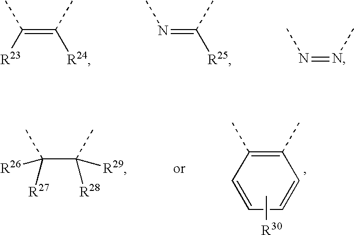
wherein

each of R²³, R²⁴, and R²⁵ is, independently, H, C₁-C₆ alkyl, halogen, or trifluoromethyl, each of R²⁶, R²⁷, R²⁸, and R²⁹ is, independently, H or C₁-C₆ alkyl, and R³⁰ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,

each of R¹² and R¹³ is, independently, H, Cl, Br, OH, OCH₃, OCF₃, NO₂, and NH₂, or R¹² and R¹³ together form a single bond.

In certain embodiments, A is

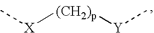

each of X and Y is independently O or NH;

p is an integer between 2 and 6, inclusive; and

m and n are, independently, integers between 0 and 2, inclusive, wherein the sum of m and n is greater than 0.

In certain other embodiments, A is

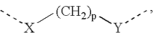

each of X and Y is independently O or NH,

p is an integer between 2 and 6, inclusive, each of m and n is 0, and

each of R¹⁰ and R¹¹ is, independently, selected from the group represented by

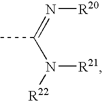

wherein R²¹ is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R²² is H, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl C₁-C₆ alkoxy), carbo(C₆-C₁₈ aryloxy), or C₆-C₁₈ aryl, and R²⁰ is H, OH, or C₁-C₆ alkyloxy, or R²⁰ and R²¹ together represent

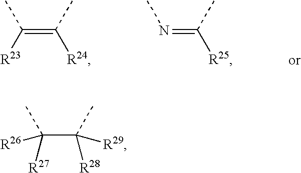

wherein each of R²³, R²⁴, and R²⁵ is, independently, H, C₁-C₆ alkyl, halogen, or trifluoromethyl, each of R²⁶, R²⁷, and R²⁸ is, independently, H or C₁-C₆ alkyl, and R²⁹ is C₁-C₆ alkyl, C₁-C₆ alkyloxy, or trifluoromethyl.

In certain other embodiments, A is

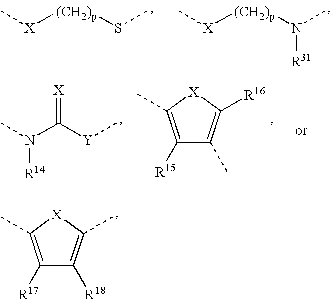

each of X and Y is, independently, O, NR¹⁹, or S,

each of R¹⁴ and R¹⁹ is, independently, H or C₁-C₆ alkyl,

each of R¹⁵, R¹⁶, R¹⁷, and R¹⁸ is, independently, H, C₁-C₆ alkyl, halogen, C₁-C₆ alkyloxy, C₆-C₁₈ aryloxy, or C₆-C₁₈ aryl C₁-C₆ alkyloxy,

R³¹ is C₁-C₆ alkyl,

p is an integer between 2 and 6, inclusive,

each of m and n is, independently, an integer between 0 and 2, inclusive,

each of R¹⁰ and R¹¹ is, independently, selected from the group represented by

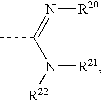

wherein R²¹ is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R²² is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R²⁰ is H, OH, or C₁-C₆ alkyloxy, or R²⁰ and R²¹ together represent

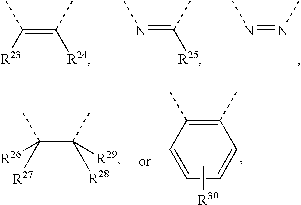

wherein each of R²³, R²⁴, and R²⁵ is, independently, H, C₁-C₆ alkyl, halogen, or trifluoromethyl, each of R²⁶, R²⁷, R²⁸, and R²⁹ are, independently, H or C₁-C₆ alkyl, and R³⁰ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl.

Other analogs include stilbamidine (G-1) and hydroxystilbamidine (G-2), and their indole analogs (e.g., G-3).

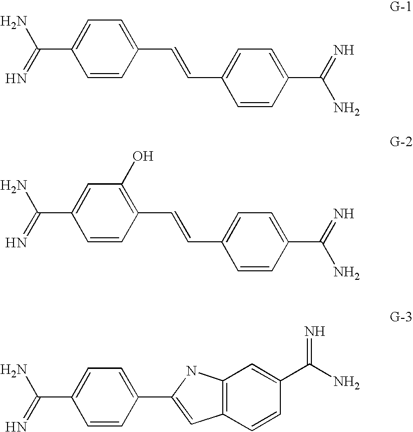

Each amidine moiety in G-1, G-2, or G-3 may be replaced with one of the moieties depicted in formula (XXIV) above as

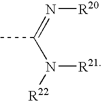

As is the case for pentamidine, salts of stilbamidine and its related compounds are also useful in the method of the invention. Preferred salts include, for example, dihydrochloride and methanesulfonate salts.

Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, or U.S. Patent Application Publication Nos. US 2001/0044468 A1 and US 2002/0019437 A1, each of which is in its entirety incorporated by reference.

Exemplary analogs are 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5[bis-{4-(2-tetrahydropyrimidinyl)}phenyl]-3-(p-tolyloxy)furan, 2,5 [bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorene, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3 [(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan. Methods for making any of the foregoing compounds are described in U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, an U.S. Patent Application Publication Nos. US 2001/0044468 A1 and US 2002/0019437 A1.

In certain embodiments, the compound of formula (XXIV) is propamidine, butamidine, heptamidine, nonamidine, stilbamidine, hydroxystilbamidine, diminazene, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, or 2,4-bis(4-amidinophenyl)thiophene-bis-O-methylamidoxime.

In certain embodiment, the compound of formula (XXIV) is pentamidine, 2,5-bis(4-amidinophenyl)furan, or 2,5-bis(4-amidinophenyl)furan-bis-O-methylamidoxime.

In certain embodiments, the second compound of drug combinations can be a functional analog of pentamidine, such as netropsin, distamycin, bleomycin, actinomycin, daunorubicin, or a compound that falls within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, or U.S. Patent Application Publication Nos. US 2001/0044468 A1 and US 2002/0019437 A1.

Pentamidine Metabolites

Pentamidine metabolites are also useful in the antiproliferative combination of the invention. Pentamidine is rapidly metabolized in the body to at least seven primary metabolites. Some of these metabolites share one or more activities with pentamidine. It is likely that some pentamidine metabolites will have anti-cancer activity when administered in combination with an antiproliferative agent. Seven pentamidine metabolites (H-1 through H-7) are shown below.

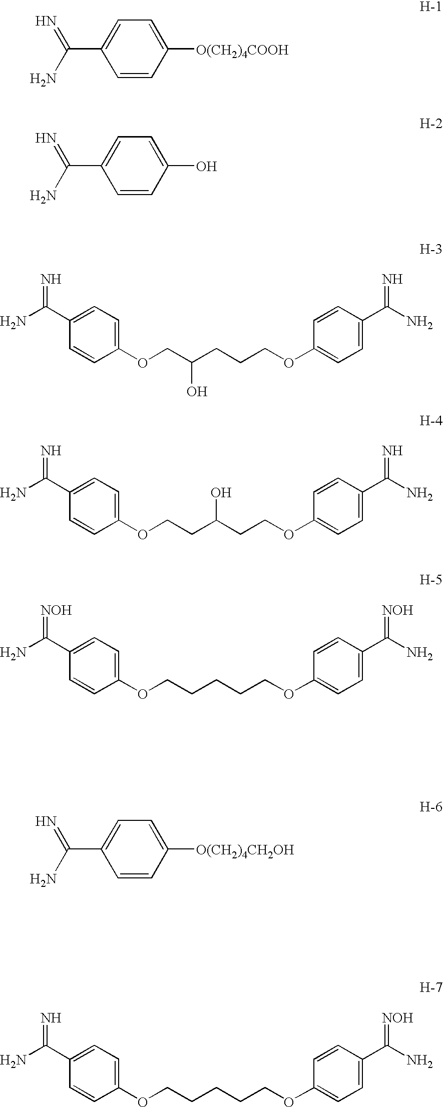

In certain embodiments, pentamidine, or analogs, derivatives, or metabolites thereof, have an antibiotic activity.

Antiproliferative Agents

In certain embodiments, an antiproliferative agent may be further included in the drug combinations that comprise (1) pentamidine (or its analog) and (2) chlorpromazine or its analogue). Antiproliferative agents are described above. Such agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors. In certain embodiments, the antiproliferative agent is a Group A antiproliferative agent as described below in the section describing combinations comprising pentamidine and antiproliferative agents (e.g., an agent listed in Table 4).

Exemplary Drug Combinations

In certain embodiments, the drug combinations of the present invention may comprise (a) a first compound selected from the group consisting of prochlorperazine, perphenazine, mepazine, methotrimeprazine, acepromazine, thiopropazate, perazine, propiomazine, putaperazine, thiethylperazine, methopromazine, chlorfenethazine, cyamemazine, perphenazine, norchlorpromazine, trifluoperazine, thioridazine (or a salt of any of the above), and dopamine D2 antagonists (e.g., sulpride, pimozide, spiperone, ethopropazine, clebopride, bupropion, and haloperidol), and (b) a second compound selected from the group consisting of pentamidine, propamidine, butamidine, heptamidine, nonamidine, stilbamidine, hydroxystilbamidine, diminazene, benzamidine, phenamidine, dibrompropamidine, 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzo[furan, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-am idino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-[1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorine, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan, or a salt of any of the above.

In certain embodiments, drug combinations may comprise (1) a first compound selected from the group consisting of acepromazine, chlorfenethazine, cyamemazine, enanthate, fluphenazine, mepazine, methotrimeprazine, methoxypromazine, norchlorpromazine, perazine, perphenazine, prochlorperazine, promethazine, propiomazine, putaperazine, thiethylperazine, thiopropazate, thioridazine, trifluoperazine, triflupromazine, and a pharmaceutically active or acceptable salt thereof, and (2) a second compound selected from the group consisting of propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, or a pharmaceutically acceptable salt thereof.

In certain embodiments, drug combinations may comprise (1) a first compound selected from the group consisting of chlorpromazine, perphenazine or promethazine, and a pharmaceutically active or acceptable salt thereof, and (2) a second compound selected from the group consisting of pentamidine, propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, or a pharmaceutically acceptable salt thereof.

In certain embodiments, the drug combination comprises (1) a compound of formula (XXIII) selected from chlorpromazine, perphenazine or promethazine and (2) a compound of formula (XXIV) selected from pentamidine, 2,5-bis(4-amidinophenyl)furan, or 2,5-bis(4-amidinophenyl)furan-bis-O-methylamidoxime.

In certain embodiments, drug combinations may comprise (1) an inhibitor of protein kinase C, and (2) a compound of formula (XXIV).

In certain embodiments, drug combinations may comprise (1) a compound of formula (XXIII), and (2) an endo-exonuclease inhibitor.

In certain embodiments, drug combinations may comprise (1) a compound of formula (XXIII), and (2) a PRL phosphatase inhibitor or a PTP1B inhibitor.

In certain embodiments, drug combinations may comprise chlorpromazine and pentamidine.

Combinations Comprising Benzimidazoles and Antiprotozoal Drugs

In certain embodiments, the drug combinations according to the present invention may comprise a benzimidazole (e.g., albendazole, mebendazole, and oxibendazole, including their structural or function analogs, salts and metabolites) and an antiprotozoal drug. In certain other embodiments, the above drug combinations may further comprise one or more antiproliferative agents (e.g., those in Table 4.

In certain embodiments, the drug combinations according to the present invention may comprise benzimidazole (e.g., albendazole, mebendazole, and oxibendazole, including their structural or function analog and metabolites) and an antiproliferative agent.

In certain embodiments, the drug combinations according to the present invention may comprise an antiprotozoal drug and an antiproliferative agent.

Benzimidazoles

Benzimidazoles that are useful in the antiproliferative combination of the invention include compounds having the general formula (XXV): 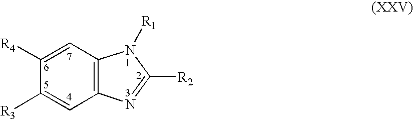
wherein:

R₁ is selected from the group consisting of H and C₁₋₁₀ alkyl or C₂₋₁₀ alkenyl that is unsubstituted or substituted by one or more substitutents selected from the group consisting of aryl, heteroaryl, heterocyclyl, OC₁₋₁₀ alkyl, O(C₁₋₁₀)₀₋₁-aryl, O—(C₁₋₁₀alkyl)₀₋₁-heteroaryl, O(C₁₋₁₀ alkyl)₀₋₁-heterocyclyl, C₁₋₁₀ alkoxycarbonyl, S(O)₀₋₂—C₁₋₁₀ alkyl, S(O)₀₋₂—(C₁₋₁₀ alkyl)₀₋₁-aryl, S(O)₀₋₂—(C₁₋₁₀ alkyl)₀₋₁-heteroaryl, S(O)₀₋₂—(C₁₋₁₀ alkyl)₀₋₁-heterocyclyl, N(R₁₃)₂, OR₁₃, oxo, cyano, halo, NO₂, OH, and SH; R₂ is selected from the group consisting of:

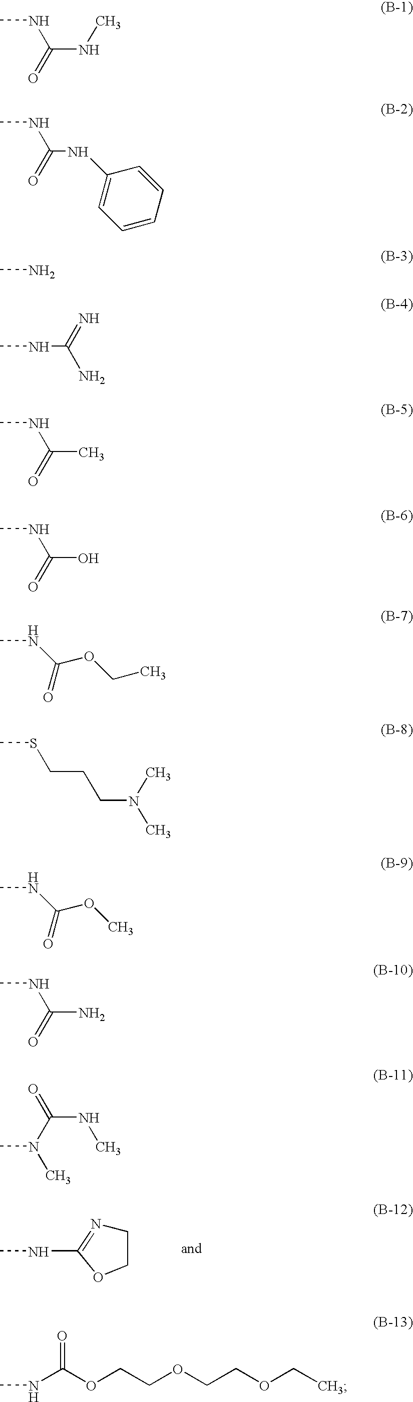

each of R₃ and R₄ is independently selected from the group consisting of H, halo, NO₂, OH, SH, OC₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂—C₁₋₁₀ alkyl, S(O)₀₋₂—(C₁₋₁₀ alkyl)₀₋₁-aryl, S(O)₀₋₂—(C₁₋₁₀ alkyl)₀₋₁-heteroaryl, S(O)₀₋₂—(C₁₋₁₀ alkyl)₀₋₁-heterocyclyl, and C₁₋₁₀ alkyl or C₂₋₁₀ alkenyl that is unsubstituted or substituted by one or more substitutents selected from the group consisting of aryl, heteroaryl, heterocyclyl, O—C₁₋₁₀ alkyl, O(C₁₋₁₀ alkyl)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂—C₁₋₁₀ alkyl, S(O)₀₋₂—(C₁₋₁₀ alkyl)₀₋₁-aryl, S(O)₀₋₂—(C₁₋₁₀ alkyl)₀₋₁-heteroaryl, S(O)₀₋₂—(C₁₋₁₀ alkyl)₀₋₁-heterocyclyl, N(R₁₃)₂, OR₁₃, oxo, cyano, halogen, NO₂, OH, and SH; and each R₁₃ is selected from the group consisting of H and C₁₋₁₀ alkyl or C₂₋₁₀ alkenyl that is unsubstituted or substituted by one or more substitutents selected from the group consisting of aryl, heteroaryl, heterocyclyl, O—C₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, oxo, cyano, halo, NO₂, OH, and SH.

Examples of substitutents R₁, R₃, and R₄ are provided below.

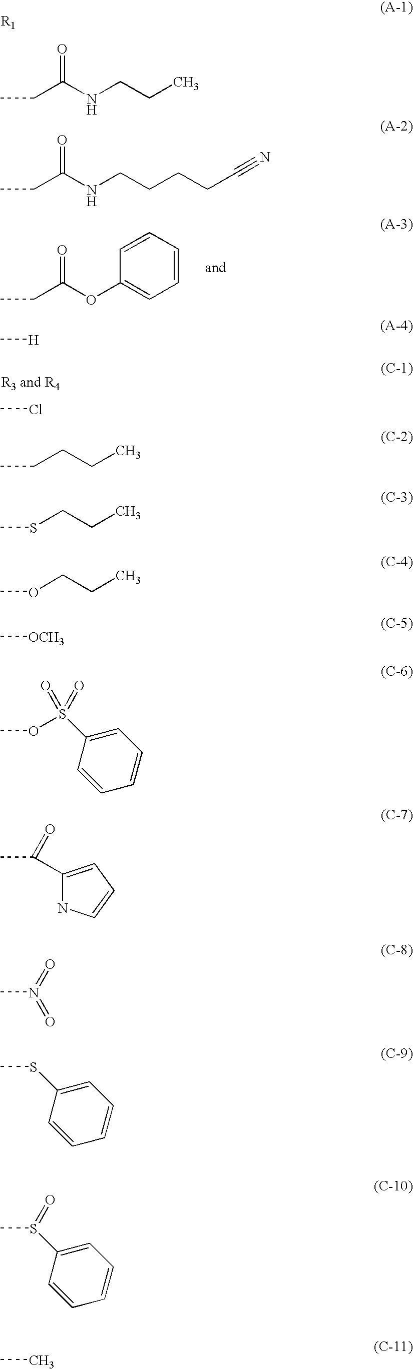

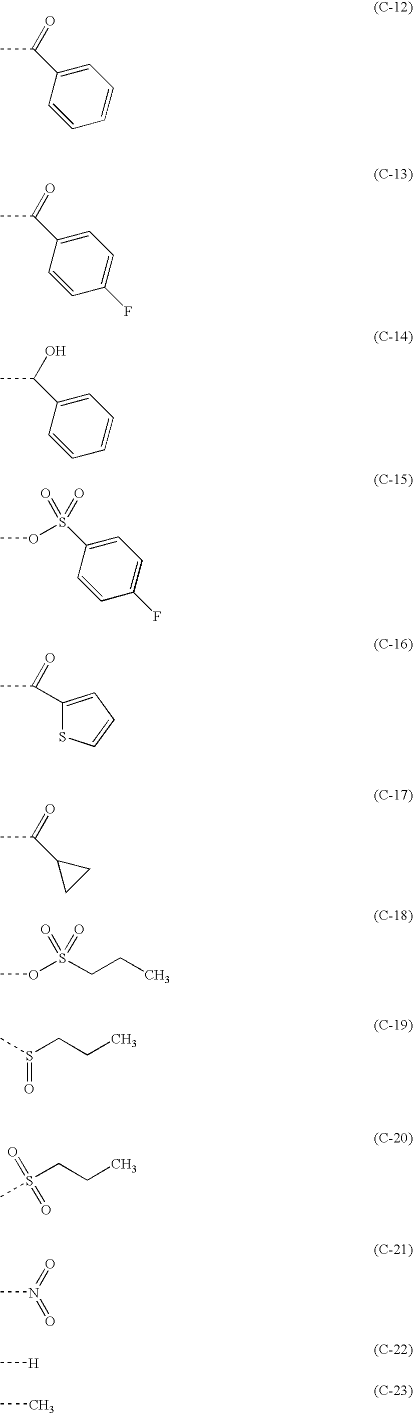

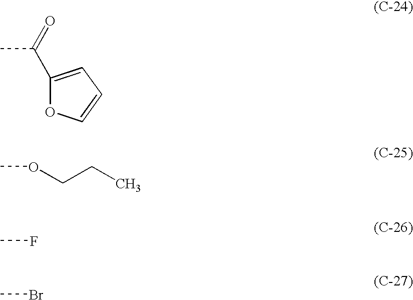

Albendazole

One of the most commonly prescribed members of the benzimidazole family is albendazole, which has the structure:

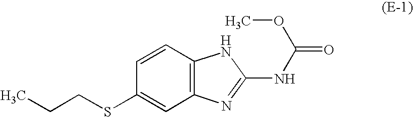

Albendazole is currently available as an oral suspension and in tablets.

Albendazole Metabolites

Albendazole undergoes metabolic transformation into a number of metabolites that may be therapeutically active; these metabolites may be substituted for albendazole in the antiproliferative combination of the invention. The metabolism of albendazole can yield, for example, albendazole sulfonate, albendazole sulfone, and albendazole sulfoxide.

Benzimidazole Analogs

Analogs of benzimidazoles include benzothioles and benzoxazoles having the structure of formula (XXVI): 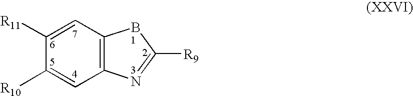
wherein: B is O or S; R₉ is selected from the group consisting of:

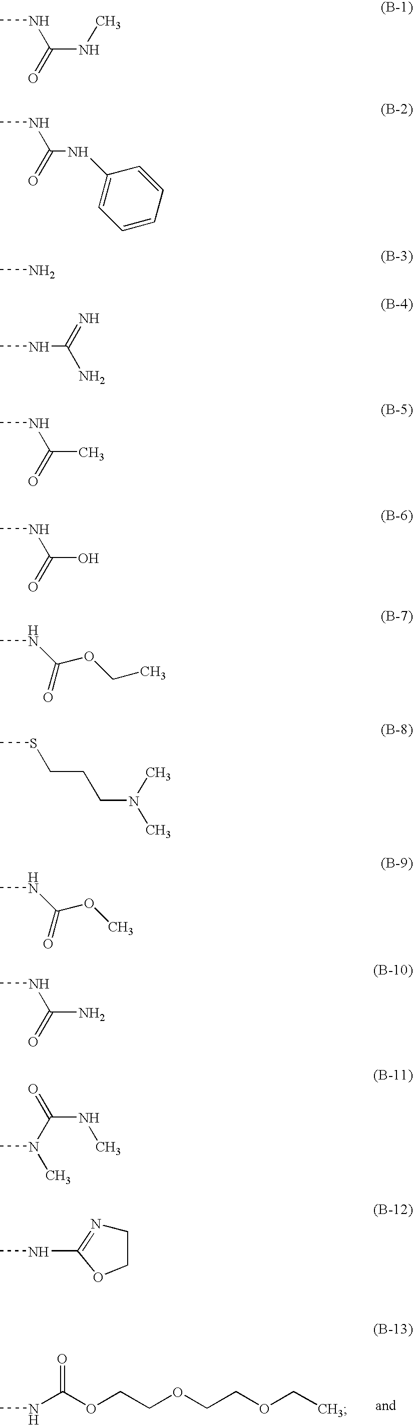

and each of R₁₀ and R₁₁ is independently selected from the group consisting of H,

halo, NO₂, OH, SH, OC₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂—C₁₋₁₀ alkyl, S(O)₀₋₂—(C₁₋₁₀ alkyl)₀₋₁-aryl, S(O)₀₋₂—(C₁₋₁₀ alkyl)₀₋₁ heteroaryl, S(O)₀₋₂—(C₁₋₁₀ alkyl)₀₋₁-heterocyclyl, and C₁₋₁₀ alkyl or C₂₋₁₀ alkenyl that is unsubstituted or substituted by one or more substitutents selected from the group consisting of aryl, heteroaryl, heterocyclyl, OC₁₋₁₀ alkyl, O(C₁₋₁₀ alkyl)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂—C₁₋₁₀ alkyl, S(O)₀₋₂—(C₁₋₁₀ alkyl)₀₋₁-aryl, S(O)₀₋₂—(C₁₋₁₀ alkyl)₀₋₁-heteroaryl, S(O)₀₋₂—(C₁₋₁₀ alkyl)₀₋₁-heterocyclyl, N(R₁₃)₂, OR₁₃, oxo, cyano, halo, NO₂, OH, and SH; and each R₁₃ is independently selected from the group consisting of H and C₁₋₁₀ alkyl or C₂₋₁₀ alkenyl that is unsubstituted or substituted by one or more substitutents selected from the group consisting of aryl, heteroaryl, heterocyclyl, OC₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, oxo, cyano, halo, NO₂, OH, and SH.

Some benzimidazoles and benzimidazole analogs fit the following formula (XXVII).

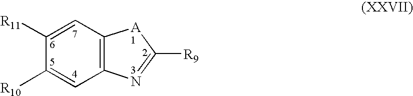

wherein A is selected from the group consisting of O, S, and NR₁₂; R₉R₁₀, R₁₁, and R₁₃ are as described above for formula (XXVII); and R₁₂ is selected from the group consisting of H and C₁₋₁₀ alkyl or C₂₋₁₀ alkenyl that is unsubstituted or substituted by one or more substitutents selected from the group consisting of aryl, heteroaryl, heterocyclyl, OC₁₋₁₀ alkyl, O(C₁₋₁₀)₀₋₁-aryl, O(C₁₋₁₀ alkyl)₀₋₁-heteroaryl, O(C₁₋₁₀ alkyl)₀₋₁-heterocyclyl, C₁₋₁₀ alkoxycarbonyl, S(O)₀₋₂—C₁₋₁₀ alkyl, S(O)₀₋₂—(C₁₋₁₀ alkyl)₀₋₁-aryl, S(O)₀₋₂—(C₁₋₁₀ alkyl)₀₋₁-heteroaryl, S(O)₀₋₂—(C₁₋₁₀ alkyl)₀₋₁-heterocyclyl, N(R₁₃)₂, OR₁₃, oxo, cyano, halo, NO₂, OH, and SH.

Exemplary Benzimidazoles and their Analogs

In certain embodiments, benzimidales or its analogs useful in the present invention may be selected from the group consisting of a first compound selected from albendazole; albendazole sulfonate; albendazole sulfone; albendazole sulfoxide; astemizole; benomyl; 2-benzimidazolylurea; benzthiazuron; cambendazole; cyclobendazole; domperidone; droperidol; fenbendazole; flubendazole; frentizole; 5-hydroxymebendazole; lobendazole; luxabendazole; mebendazole; methabenzthiazuron; mercazole; midefradil; nocodozole; omeprazole; oxfendazole; oxibendazole; parbendazole; pimozide; and tioxidazole (or a salt of any of the above); NSC 181928 (ethyl 5-amino-1,2-dihydro-3-[(N-methylanilino)methyl]-pyrido[3,4-b]pyrazin-7-ylcarbamate); TN-16 (3-(1-anilinoethylidene)-5-benzyl-pyrrodiline-2,4-dione); and pharmaceutically active or acceptable salts thereof.

It will be understood by those in the art that the compounds are also useful when formulated as salts. For example, benzimidazole salts include halide, sulfate, nitrate, phosphate, and phosphinate salts.

Pentamidine and its Analogs

Pentamidine

Pentamidine is described in detail above.

Pentamidine Analogs

Aromatic diamidino compounds can replace pentamidine in the antiproliferative combination of the invention. These compounds are referred to as pentamidine analogs. Examples are propamidine, butamidine, heptamidine, and nonamidine, all of which, like pentamidine, exhibit antipathogenic or DNA binding properties. Other analogs (e.g., stilbamidine and indole analogs of stilbamidine, hydroxystilbamidine, diminazene, benzamidine, dibrompropamidine, 1,3-bis(4-amidino-2-methoxyphenoxy) propane (DAMP), netropsin, distamycin, phenamidine, amicarbalide, bleomycin, actinomycin, and daunorubicin) also exhibit properties in common with pentamidine.

Suitable analogs include those falling within formula (XXVIII).

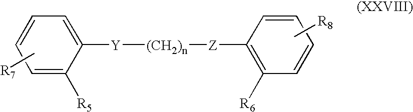

wherein each of Y and Z is, independently, O or N; each of R₅ and R₆ is, independently, H, OH, Cl, Br, F, OCH₃, OCF₃, NO₂, or NH₂; n is an integer between 2 and 6, inclusive; and each of R₇ and R₉ is, independently, at the meta or para position and is selected from any one of the following structures D-1, D2, D-3, D-4, D-5, and D-6:

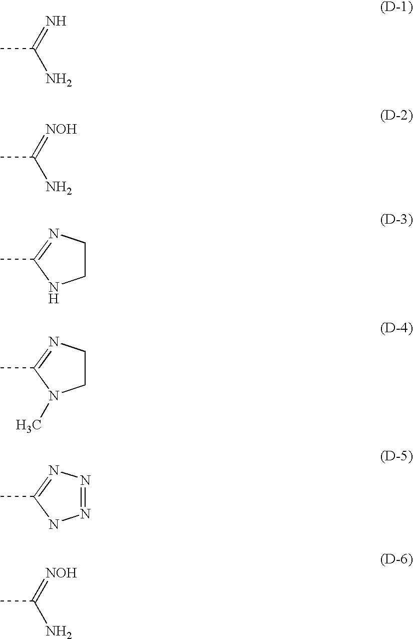

Other suitable pentamidine analogs include stilbamidine (G-1) and hydroxystilbamidine (G-2), and their indole analogs (e.g., G-3):

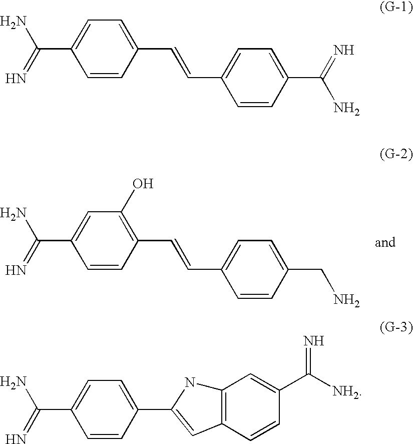

Each amidine moiety may independently be replaced with one of the moieties depicted as D-2, D-3, D-4, D-5, or D-6 above. As is the case for the benzimidazoles and pentamidine, salts of stilbamidine, hydroxystilbamidine, and their indole derivatives are also useful in the method of the invention. Preferred salts include, for example, dihydrochloride and methanesulfonate salts.

Still other analogs are those that fall within a formula provided in any of U.S. Pat. Nos. 5,428,051; 5,521,189; 5,602,172; 5,643,935; 5,723,495; 5,843,980; 6,172,104; and 6,326,395, or U.S. Patent Application Publication No. US 2002/0019437 A1, each of which is in its entirety incorporated by reference. Exemplary analogs include 1,5-bis-(4′-(N-hydroxyamidino)phenoxy) pentane; 1,3-bis-(4′-(N-hydroxyamidino)phenoxy) propane; 1,3-bis-(2′-methoxy-4′-(N-hydroxyamidino)phenoxy)propane; 1,4-bis-(4′-(N-hydroxyamidino)phenoxy) butane; 1,5-bis-(4′-(N-hydroxyamidino) phenoxy)pentane; 1,4-bis-(4′-(N-hydroxyamidino)phenoxy)butane; 1,3-bis-(4′-(4-hydroxyamidino)phenoxy) propane; 1,3-bis-(2′-methoxy-4′-(N-hydroxyamidino) phenoxy)propane; 2,5-bis-[4-amidinophenyl]furan; 2,5-bis-[4-amidinophenyl]furan bis-amidoxime; 2,5-bis-[4-amidinophenyl]furan bis-O-methylamidoxime; 2,5-bis-[4-amidinophenyl]furan bis-O-ethylamidoxime; 2,8-diamidinodibenzothiophene; 2,8-bis-(N-isopropylamidino) carbazole; 2,8-bis-(N-hydroxyamidino)carbazole; 2,8-bis-(2-imidazolinyl)dibenzothiophene; 2,8-bis-(2-imidazolinyl)-5,5-dioxodibenzothiophene; 3,7-diamidinodibenzothiophene; 3,7-bis-(N-isopropylamidino)dibenzothiophene; 3,7-bis-(N-hydroxyamidino) dibenzothiophene; 3,7-diaminodibenzothiophene; 3,7-dibromodibenzothiophene; 3,7-dicyanodibenzothiophene; 2,8-diamidinodibenzofuran; 2,8-di(2-imidazolinyl) dibenzofuran; 2,8-di(N-isopropylamidino)dibenzofuran; 2,8-di(N-hydroxylamidino)dibenzofuran; 3,7-di(2-imidazolinyl)dibenzofuran; 3,7-di(isopropylamidino)dibenzofuran; 3,7-di(A-hydroxylamidino)dibenzofuran; 2,8-dicyanodibenzofuran; 4,4′-dibromo-2,2′-dinitrobiphenyl; 2-methoxy-2′-nitro-4,4′-dibromobiphenyl; 2-methoxy-2′-amino-4,4′-dibromobiphenyl; 3,7-dibromo-dibenzofuran; 3,7-dicyano-dibenzofuran; 2,5-bis-(5-amidino-2-benzimidazolyl) pyrrole; 2,5-bis-[5-(2-imidazolinyl)-2-benzimidazolyl]pyrrole; 2,6-bis-[5-(2-imidazolinyl)-2-benzimidazolyl]pyridine; 1-methyl-2,5-bis-(5-amidino-2-benzimidazolyl)pyrrole; 1-methyl-2,5-bis-[5-(2-imidazolyl)-2-benzimidazolyl]pyrrole; 1-methyl-2,5-bis-[5-(1,4,5,6-tetrahydro-2-pyrimidinyl)-2-benzimidazolyl]pyrrole; 2,6-bis-(5-amidino-2-benzimidazoyl)pyridine; 2,6-bis-[5-(1,4,5,6-tetrahydro-2-pyrimidinyl)-2-benzimidazolyl]pyridine; 2,5-bis-(5-amidino-2-benzimidazolyl)furan; 2,5-bis-[5-(2-imidazolinyl)-2-benzimidazolyl]furan; 2,5-bis-(5-N-isopropylamidino-2-benzimidazolyl)furan; 2,5-bis-(4-guanylphenyl) furan; 2,5-bis(4-guanylphenyl)-3,4-dimethylfuran; 2,5-d]-p[2(3,4,5,6-tetrahydropyrimidyl)phenyl]furan; 2,5-bis-[4-(2-imidazolinyl)phenyl]furan; 2,5-[bis-{4-(2-tetrahydropyrimidinyl)}phenyl]-p(tolyloxy)furan; 2,5-[bis{4-(2-imidazolinyl)}phenyl]3-p(tolyloxy)furan; 2,5-bis-{4-[5-(N-2-aminoethylamido) benzimidazol-2-yl]phenyl}furan; 2,5-bis[4-(3a,4,5,6,7,7a-hexahydro-1H-benzimidazol-2-yl)phenyl]furan; 2,5-bis-[4-(4,5,6,7-tetrahydro-1H-1,3-diazepin-2-yl)phenyl]furan; 2,5-bis-(4-N,N-dimethylcarboxhydrazidephenyl)furan; 2,5-bis-{4-[2-(N-2-hydroxyethyl)imidazolinyl]-phenyl}furan; 2,5-bis[4-(N-isopropylamidino)phenyl]furan; 2,5-bis-{4-[3-(dimethylaminopropyl) amidino]phenyl}furan; 2,5-bis-{4-[N-(3-aminopropyl)amidino]phenyl}furan; 2,5-bis-[2-(imidzaolinyl)phenyl]-3,4-bis(methoxymethyl)furan; 2,5-bis-[4-N-(dimethylaminoethyl)guanyl]phenylfuran; 2,5-bis-{4-[(N-2-hydroxyethyl) guanyl]phenyl}furan; 2,5-bis-[4-N-(cyclopropylguanyl)phenyl]furan; 2,5-bis-[4-(N,N-diethylaminopropyl)guanyl]phenylfuran; 2,5-bis-{4-[2-(N-ethylimidazolinyl)]phenyl}furan; 2,5-bis-{4-[N-(3-pentylguanyl)]}phenylfuran; 2,5-bis-[4-(2-imidazolinyl)phenyl]-3-methoxyfuran; 2,5-bis-[4-(N-isopropylamidino)phenyl]-3-methylfuran; bis-[5-amidino-2-benzimidazolyl]methane; bis-[5-(2-imidazolyl)-2-benzimidazolyl]methane; 1,2-bis-[5-amidino-2-benzimidazolyl]ethane; 1,2-bis-[5-(2-imidazolyl)-2-benzimidazolyl]ethane; 1,3-bis-[5-amidino-2-benzimidazolyl]propane; 1,3-bis-[5-(2-imidazolyl)-2-benzimidazolyl]propane; 1,4-bis-[5-amidino-2-benzimidazolyl]propane; 1,4-bis-[5-(2-imidazolyl)-2-benzimidazolyl]butane; 1,8-bis-[5-amidino-2-benzimidazolyl]octane; trans-1,2-bis-[5-amidino-2-benzimidazolyl]ethene; 1,4-bis-[5-(2-imidazolyl)-2-benzimidazolyl]1-butene; 1,4-bis-[5-(2-imidazolyl)-2-benzimidazolyl]2-butene; 1,4-bis-[5-(2-imidazolyl)-2-benzimidazolyl]1-methylbutane; 1,4-bis-[5-(2-imidazolyl)-2-benzimidazolyl]2-ethylbutane; 1,4-bis-[5-(2-imidazolyl)-2-benzimidazolyl]1-methyl-1-butene; 1,4-bis-[5-(2-imidazolyl)-2-benzimidazolyl]2,3-diethyl-2-butene; 1,4-bis-[5-(2-imidazolyl)-2-benzimidazolyl]1,3-butadiene; 1,4-bis-[5-(2-imidazolyl)-2-benzimidazolyl]2-methyl-1,3-butadiene; bis-[5-(2-pyrimidyl)-2-benzimidazolyl]methane; 1,2-bis-[5-(2-pyrimidyl)-2-benzimidazolyl]ethane; 1,3-bis-[5-amidino-2-benzimidazolyl]propane; 1,3-bis-[5-(2-pyrimidyl)-2-benzimidazolyl]propane; 1,4-bis-[5-(2-pyrimidyl)-2-benzimidazolyl]butane; 1,4-bis-[5-(2-pyrimidyl)-2-benzimidazolyl]1-butene; 1,4-bis-[[5-(2-pyrimidyl)-2-benzimidazolyl]2-butene; 1,4-bis-[5-(2-pyrimidyl)-2-benzimidazolyl]1-methylbutane; 1,4-bis-[5-(2-pyrimidyl)-2-benzimidazolyl]2-ethylbutane; 1,4-bis-[5-(2-pyrimidyl)-2-benzimidazolyl]1-methyl-1-butene; 1,4-bis-[5-(2-pyrimidyl)-2-benzimidazolyl]2,3-diethyl-2-butene; 1,4-bis-[5-(2-pyrimidyl)-2-benzimidazolyl]1,3-butadiene; and 1,4-bis-[5-(2-pyrimidyl)-2-benzimidazolyl]2-methyl-1,3-butadiene; 2,4-bis-(4-guanylphenyl)-pyrimidine; 2,4-bis-(4-imidazolin-2-yl)-pyrimidine; 2,4-bis-[(tetrahydropyrimidinyl-2-yl)phenyl]pyrimidine; 2-(4-[N-i-propylguanyl]phenyl)-4-(2-methoxy-4-[N-i-propylguanyl]phenyl)pyrimidine; 4-(N-cyclopentylamidino)-1,2-phenylene diamine; 2,5-bis-[2-(5-amidino)benzimidazoyl]furan; 2,5-bis-[2-{5-(2-imidazolino)}benzimidazoyl]furan; 2,5-bis-[2-(5-N-isopropylamidino) benzimidazoyl]furan; 2,5-bis-[2-(5-N-cyclopentylamidino) benzimidazoyl]furan; 2,5-bis[2-(5-amidino)benzimidazoyl]pyrrole; 2,5-bis-[2-{5-(2-imidazolino)}benzimidazoyl]pyrrole; 2,5-bis[2-(5-N-isopropylamidino)benzimidazoyl]pyrrole; 2,5-bis-[2-(5-N-cyclopentylamidino)benzimidazoyl]pyrrole; 1-methyl-2,5-bis-[2-(5-amidino)benzimidazoyl]pyrrole; 2,5-bis-[2-{5-(2-imidazolino)}benzimidazoyl]-1-methylpyrrole; 2,5-bis[2-(5-N-cyclopentylamidino) benzimidazoyl]1-methylpyrrole; 2,5-bis-[2-(5-N-isopropylamidino) benzimidazoyl]thiophene; 2,6-bis-[2-{5-(2-imidazolino)}benzimidazoyl]pyridine; 2,6-bis-[2-(5-amidino)benzimidazoyl]pyridine; 4,4′-bis-[2-(5-N-isopropylamidino) benzimidazoyl]1,2-diphenylethane; 4,4′-bis-[2-(5-N-cyclopentylamidino) benzimidazoyl]-2,5-diphenylfuran; 2,5-bis-[2-(5-amidino) benzimidazoyl]benzo[b]furan; 2,5-bis-[2-(5-N-cyclopentylamidino)benzimidazoyl]benzo[b]furan; 2,7-bis-[2-(5-N-isopropylamidino)benzimidazoyl]fluorine; 2,5-bis-[4-(3-(N-morpholinopropyl)carbamoyl)phenyl]furan; 2,5-bis-[4-(2-N,N-dimethylaminoethylcarbamoyl)phenyl]furan; 2,5-bis-[4-(3-N,N-dimethylaminopropylcarbamoyl)phenyl]furan; 2,5-bis-[4-(3-N-methyl-3-N-phenylaminopropylcarbamoyl)phenyl]furan; 2,5-bis-[4-(3-N,N⁸,N¹¹-trimethylaminopropylcarbamoyl)phenyl]furan; 2,5-bis-[3-amidinophenyl]furan; 2,5-bis-[3-(N-isopropylamidino)amidinophenyl]furan; 2,5-bis[3[(N-(2-dimethylaminoethyl)amidino]phenylfuran; 2,5-bis-[4-(N-2,2,2-trichloroethoxycarbonyl)amidinophenyl]furan; 2,5-bis-[4-(N-thioethylcarbonyl) amidinophenyl]furan; 2,5-bis-[4-(N-benzyloxycarbonyl)amidinophenyl]furan; 2,5-bis[4-(N-phenoxycarbonyl)amidinophenyl]furan; 2,5-bis-[4-(N-(4-fluoro)-phenoxycarbonyl)amidinophenyl]furan; 2,5-bis-[4-(N-(4-methoxy) phenoxycarbonyl)amidinophenyl]furan; 2,5-bis-[4(1-acetoxyethoxycarbonyl) amidinophenyl]furan; and 2,5-bis-[4-(N-(3-fluoro)phenoxycarbonyl) amidinophenyl]furan. Methods for making any of the foregoing compounds are described in U.S. Pat. Nos. 5,428,051; 5,521,189; 5,602,172; 5,643,935; 5,723,495; 5,843,980; 6,172,104; and 6,326,395, or U.S. Patent Application Publication No. US 2002/0019437 A1.

Pentamidine Metabolites

Pentamidine metabolites are also useful in the antiproliferative combination of the invention. Pentamidine is rapidly metabolized in the body to at least seven primary metabolites. Some of these metabolites share one or more activities with pentamidine. It is likely that some pentamidine metabolites will exhibit antiproliferative activity when combined with a benzimidazole or an analog thereof.

Seven pentamidine metabolites are shown below.

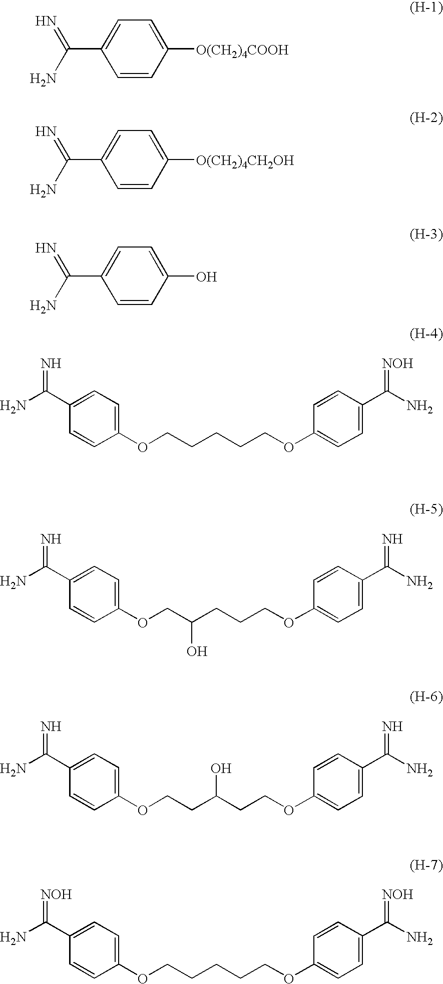

It will be understood by those in the art that the compounds are also useful when formulated as salts. For example, pentamidine salts include the isethionate salt, the platinum salt, the dihydrochloride salt, and the dimethanesulfonate salt (see, for example, Mongiardo et al., Lancet 2:108, 1989).

Exemplary Drug Combinations

In certain embodiments, the drug combinations according to the present invention may comprises (a) a first compound selected from albendazole; albendazole sulfonate; albendazole sulfone; albendazole sulfoxide; astemizole; benomyl; 2-benzimidazolylurea; benzthiazuron; cambendazole; cyclobendazole; domperidone; droperidol; fenbendazole; flubendazole; frentizole; 5-hydroxymebendazole; lobendazole; luxabendazole; mebendazole; methabenzthiazuron; mercazole; midefradil; nocodozole; omeprazole; oxfendazole; oxibendazole; parbendazole; pimozide; and tioxidazole (or a salt of any of the above); NSC 181928 (ethyl 5-amino-1,2-dihydro-3-[(N-methylanilino)methyl]-pyrido[3,4-b]pyrazin-7-ylcarbamate); and TN-16 (3-(1-anilinoethylidene)-5-benzyl-pyrrodiline-2,4-dione); and (b) a second compound selected from pentamidine; propamidine; butamidine; heptamidine; nonamidine; stilbamidine; hydroxystilbamidine; diminazene; benzamidine; phenamidine; dibrompropamidine; 1,3-bis-(4-amidino-2-methoxyphenoxy) propane; phenamidine; amicarbalide; 1,5-bis-(4′-(N-hydroxyamidino)phenoxy) pentane; 1,3-bis-(4′-(N-hydroxyamidino)phenoxy) propane; 1,3-bis-(2′-methoxy-4′-(N-hydroxyamidino)phenoxy)propane; 1,4-bis-(4′-(N-hydroxyamidino)phenoxy) butane; 1,5-bis-(4′-(N-hydroxyamidino) phenoxy)pentane; 1,4-bis-(4′-(N-hydroxyamidino)phenoxy)butane; 1,3-bis-(4′-(4-hydroxyamidino)phenoxy)propane; 1,3-bis-(2′-methoxy-4′-(N-hydroxyamidino) phenoxy)propane; 2,5-bis-[4-amidinophenyl]furan; 2,5-bis-[4-amidinophenyl]furan bis-amidoxime; 2,5-bis-[4-amidinophenyl]furan bis-O-methylamidoxime; 2,5-bis-[4-amidinophenyl]furan bis-O-ethylamidoxime; 2,8-diamidinodibenzothiophene; 2,8-bis-(N-isopropylamidino) carbazole; 2,8-bis-(N-hydroxyamidino)carbazole; 2,8-bis-(2-imidazolinyl)dibenzothiophene; 2,8-bis-(2-imidazolinyl)-5,5-dioxodibenzothiophene; 3,7-diamidinodibenzothiophene; 3,7-bis-(N-isopropylamidino)dibenzothiophene; 3,7-bis-(N-hydroxyamidino) dibenzothiophene; 3,7-diaminodibenzothiophene; 3,7-dibromodibenzothiophene; 3,7-dicyanodibenzothiophene; 2,8-diamidinodibenzofuran; 2,8-di(2-imidazolinyl) dibenzofuran; 2,8-di(N-isopropylamidino)dibenzofuran; 2,8-di(N-hydroxylamidino)dibenzo[t]ran; 3,7-di(2-imidazolinyl)dibenzofuran; 3,7-di(isopropylamidino)dibenzofuran; 3,7-di(A-hydroxylamidino)dibenzofuran; 2,8-dicyanodibenzofuran; 4,4′-dibromo-2,2′-dinitrobiphenyl; 2-methoxy-2′-nitro-4,4′-dibromobiphenyl; 2-methoxy-2′-amino-4,4′-dibromobiphenyl; 3,7-dibromo-dibenzofuran; 3,7-dicyano-dibenzofuran; 2,5-bis-(5-amidino-2-benzimidazolyl) pyrrole; 2,5-bis-[5-(2-imidazolinyl)-2-benzimidazolyl]pyrrole; 2,6-bis-[5-(2-imidazolinyl)-2-benzimidazolyl]pyridine; 1-methyl-2,5-bis-(5-amidino-2-benzimidazolyl)pyrrole; 1-methyl-2,5-bis-[5-(2-imidazolyl)-2-benzimidazolyl]pyrrole; 1-methyl-2,5-bis-[5-(1,4,5,6-tetrahydro-2-pyrimidinyl)-2-benzimidazolyl]pyrrole; 2,6-bis-(5-amidino-2-benzimidazoyl)pyridine; 2,6-bis-[5-(1,4,5,6-tetrahydro-2-pyrimidinyl)-2-benzimidazolyl]pyridine; 2,5-bis-(5-amidino-2-benzimidazolyl)furan; 2,5-bis-[5-(2-imidazolinyl)-2-benzimidazolyl]furan; 2,5-bis-(5-N-isopropylamidino-2-benzimidazolyl)furan; 2,5-bis-(4-guanylphenyl) furan; 2,5-bis(4-guanylphenyl)-3,4-dimethylfuran; 2,5-d]-p[2(3,4,5,6-tetrahydropyrimidyl)phenyl]furan; 2,5-bis-[4-(2-imidazolinyl)phenyl]furan; 2,5-[bis-{4-(2-tetrahydropyrimidinyl)}phenyl]-p(tolyloxy)furan; 2,5-[bis{4-(2-imidazolinyl)}phenyl]3-p(tolyloxy)furan; 2,5-bis-{4-[5-(N-2-aminoethylamido) benzimidazol-2-yl]phenyl}furan; 2,5-bis[4-(3a,4,5,6,7,7a-hexahydro-1H-benzimidazol-2-yl)phenyl]furan; 2,5-bis-[4-(4,5,6,7-tetrahydro-1H-1,3-diazepin-2-yl)phenyl]furan; 2,5-bis-(4-N,N-dimethylcarboxhydrazidephenyl)furan; 2,5-bis-{4-[2-(N-2-hydroxyethyl)imidazolinyl]-phenyl}furan; 2,5-bis[4-(N-isopropylamidino)phenyl]furan; 2,5-bis-{4-[3-(dimethylaminopropyl) amidino]phenyl}furan; 2,5-bis-{4-[N-(3-aminopropyl)amidino]phenyl}furan; 2,5-bis-[2-(imidzaolinyl)phenyl]-3,4-bis(methoxymethyl)furan; 2,5-bis-[4-N-(dimethylaminoethyl)guanyl]phenylfuran; 2,5-bis-{4-[(N-2-hydroxyethyl) guanyl]phenyl}furan; 2,5-bis-[4-N-(cyclopropylguanyl)phenyl]furan; 2,5-bis-[4-(N,N-diethylaminopropyl)guanyl]phenylfuran; 2,5-bis-{4-[2-(N-ethylimidazolinyl)]phenyl}furan; 2,5-bis-{4-[N-(3-pentylguanyl)]}phenylfuran; 2,5-bis-[4-(2-imidazolinyl)phenyl]-3-methoxyfuran; 2,5-bis-[4-(N-isopropylamidino)phenyl]-3-methylfuran; bis-[5-amidino-2-benzimidazolyl]methane; bis-[5-(2-imidazolyl)-2-benzimidazolyl]methane; 1,2-bis-[5-amidino-2-benzimidazolyl]ethane; 1,2-bis-[5-(2-imidazolyl)-2-benzimidazolyl]ethane; 1,3-bis-[5-amidino-2-benzimidazolyl]propane; 1,3-bis-[5-(2-imidazolyl)-2-benzimidazolyl]propane; 1,4-bis-[5-amidino-2-benzimidazolyl]propane; 1,4-bis-[5-(2-imidazolyl)-2-benzimidazolyl]butane; 1,8-bis-[5-amidino-2-benzimidazolyl]octane; trans-1,2-bis-[5-amidino-2-benzimidazolyl]ethene; 1,4-bis-[5-(2-imidazolyl)-2-benzimidazolyl]1-butene; 1,4-bis-[5-(2-imidazolyl)-2-benzimidazolyl]2-butene; 1,4-bis-[5-(2-imidazolyl)-2-benzimidazolyl]1-methylbutane; 1,4-bis-[5-(2-imidazolyl)-2-benzimidazolyl]2-ethylbutane; 1,4-bis-[5-(2-imidazolyl)-2-benzimidazolyl]1-methyl-1-butene; 1,4-bis-[5-(2-imidazolyl)-2-benzimidazolyl]2,3-diethyl-2-butene; 1,4-bis-[5-(2-imidazolyl)-2-benzimidazolyl]1,3-butadiene; 1,4-bis-[5-(2-imidazolyl)-2-benzimidazolyl]2-methyl-1,3-butadiene; bis-[5-(2-pyrimidyl)-2-benzimidazolyl]methane; 1,2-bis-[5-(2-pyrimidyl)-2-benzimidazolyl]ethane; 1,3-bis-[5-amidino-2-benzimidazolyl]propane; 1,3-bis-[5-(2-pyrimidyl)-2-benzimidazolyl]propane; 1,4-bis-[5-(2-pyrimidyl)-2-benzimidazolyl]butane; 1,4-bis-[5-(2-pyrimidyl)-2-benzimidazolyl]1-butene; 1,4-bis-[5-(2-pyrimidyl)-2-benzimidazolyl]2-butene; 1,4-bis-[5-(2-pyrimidyl)-2-benzimidazolyl]1-methylbutane; 1,4-bis-[5-(2-pyrimidyl)-2-benzimidazolyl]2-ethylbutane; 1,4-bis-[5-(2-pyrimidyl)-2-benzimidazolyl]1-methyl-1-butene; 1,4-bis-[5-(2-pyrimidyl)-2-benzimidazolyl]2,3-diethyl-2-butene; 1,4-bis-[5-(2-pyrimidyl)-2-benzimidazolyl]1,3-butadiene; and 1,4-bis-[5-(2-pyrimidyl)-2-benzimidazolyl]2-methyl-1,3-butadiene; 2,4-bis-(4-guanylphenyl)-pyrimidine; 2,4-bis-(4-imidazolin-2-yl)-pyrimidine; 2,4-bis-[(tetrahydropyrimidinyl-2-yl)phenyl]pyrimidine; 2-(4-[N-i-propylguanyl]phenyl)-4-(2-methoxy-4-[N-i-propylguanyl]phenyl)pyrimidine; 4-(N-cyclopentylamidino)-1,2-phenylene diamine; 2,5-bis-[2-(5-amidino)benzimidazoyl]furan; 2,5-bis-[2-{5-(2-imidazolino)}benzimidazoyl]furan; 2,5-bis-[2-(5-N-isopropylamidino) benzimidazoyl]furan; 2,5-bis-[2-(5-N-cyclopentylamidino) benzimidazoyl]furan; 2,5-bis[2-(5-amidino)benzimidazoyl]pyrrole; 2,5-bis-[2-{5-(2-imidazolino)}benzimidazoyl]pyrrole; 2,5-bis[2-(5-N-isopropylamidino)benzimidazoyl]pyrrole; 2,5-bis-[2-(5-N-cyclopentylamidino)benzimidazoyl]pyrrole; 1-methyl-2,5-bis-[2-(5-amidino)benzimidazoyl]pyrrole; 2,5-bis-[2-{5-(2-imidazolino)}benzimidazoyl]-1-methylpyrrole; 2,5-bis[2-(5-N-cyclopentylamidino) benzimidazoyl]1-methylpyrrole; 2,5-bis-[2-(5-N-isopropylamidino) benzimidazoyl]thiophene; 2,6-bis-[2-{5-(2-imidazolino)}benzimidazoyl]pyridine; 2,6-bis-[2-(5-amidino)benzimidazoyl]pyridine; 4,4′-bis-[2-(5-N-isopropylamidino) benzimidazoyl]1,2-diphenylethane; 4,4′-bis-[2-(5-N-cyclopentylamidino) benzimidazoyl]-2,5-diphenylfuran; 2,5-bis-[2-(5-amidino) benzimidazoyl]benzo[b]furan; 2,5-bis-[2-(5-N-cyclopentylamidino)benzimidazoyl]benzo[b]furan; 2,7-bis-[2-(5-N-isopropylamidino)benzimidazoyl]fluorine; 2,5-bis-[4-(3-(N-morpholinopropyl)carbamoyl)phenyl]furan; 2,5-bis-[4-(2-N,N-dimethylaminoethylcarbamoyl)phenyl]furan; 2,5-bis-[4-(3-N,N-dimethylaminopropylcarbamoyl)phenyl]furan; 2,5-bis-[4-(3-N-methyl-3-N-phenylaminopropylcarbamoyl)phenyl]furan; 2,5-bis-[4-(3-N,N⁸,N¹¹-trimethylaminopropylcarbamoyl)phenyl]furan; 2,5-bis-[3-amidinophenyl]furan; 2,5-bis-[3-(N-isopropylamidino)amidinophenyl]furan; 2,5-bis[3[(N-(2-dimethylaminoethyl)amidino]phenylfuran; 2,5-bis-[4-(N-2,2,2-trichloroethoxycarbonyl)amidinophenyl]furan; 2,5-bis-[4-(N-thioethylcarbonyl) amidinophenyl]furan; 2,5-bis-[4-(N-benzyloxycarbonyl)amidinophenyl]furan; 2,5-bis[4-(N-phenoxycarbonyl)amidinophenyl]furan; 2,5-bis-[4-(N-(4-fluoro)-phenoxycarbonyl)amidinophenyl]furan; 2,5-bis-[4-(N-(4-methoxy) phenoxycarbonyl)amidinophenyl]furan; 2,5-bis-[4(1-acetoxyethoxycarbonyl) amidinophenyl]furan; and 2,5-bis-[4-(N-(3-fluoro)phenoxycarbonyl) amidinophenyl]furan (or a salt of any of the above).

In certain embodiments, the above drug combinations may further comprise an antiproliferative agent.

In certain embodiments, the drug combinations may comprise a first compound as listed above and an antiproliferative agent.

In certain other embodiments, the drug combinations may comprise a second compound as listed above and an antiproliferative agent.

In certain embodiments, the drug combinations comprise a first compound selected from alberdazole, mebendazole, oxibendazole, or a salt thereof and a second compound is pentamidine or a salt thereof.

In certain embodiments, the drug combinations of the present invention may comprise albendazole and pentamidine isethionate. In certain other embodiments, the drug combinations of the present invention may comprise albendazole sulfoxide and pentamidine isethionate, mebendazole and pentamidine isethionate, or oxibendazole and pentamidine isethionate.

In certain embodiments, the drug combinations of the present invention may comprise albendazole and 2,5-bis-[4-amidinophenyl]furan bis-O-methylamidoxime.

In certain embodiments, the drug combinations of the present invention may comprise albendazole and 2,5-bis-[4-amidinophenyl]furan.

Combinations Comprising Dibucaine or Amide Local Anaesthetics Related to Bupivacaine and Vinca Alkaloids

In certain embodiments, the drug combinations according to the present invention may comprise (1) a dibucaine or amide local anaestheic related to bupivacaine (or structural or functional analogues, salts, or metabolites) and (2) a vinca alkaloid (or structural or functional analogues, salts, or metabolites). In certain embodiments, the drug combination may further comprise one or more antiproliferative agents (e.g., those listed in Table 4).

Dibucaine and Amide Local Anaesthetics Related to Bupivacaine

Compounds of Formula (XXIX)

Compounds of formula (XXIX) have the formula:

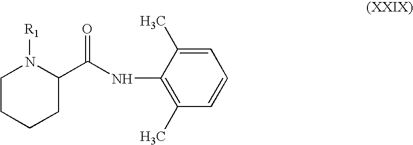

wherein R₁ is H, OH, a halide, or any branched or unbranched, substituted or unsubstituted C₁₋₁₀ alkyl, C₁₋₁₀ alkoxyalkyl, C₁₋₁₀ hydroxyalkyl, C₁₋₁₀ aminoalkyl, C₁₋₁₀ alkylaminoalkyl, C₄₋₁₀ cycloalkyl, C₅₋₈ aryl, or C₆₋₂₀ alkylaryl; most preferably R₁ is CH₃—, CH₃CH₂CH₂—, or CH₃CH₂CH₂CH₂—.

Exemplary compounds of this formula are bupivacaine (1-butyl-2′,6′-pipecoloxylidide), levobupivacaine (also called chirocaine; (S)-1-butyl-2′,6′-pipecoloxylidide), mepivacaine ((+/−)-1-methyl-2′,6′-pipecoloxylidide), and ropivacaine ((−)-1-propyl-2′,6′-pipecoloxylidide). These compounds are tertiary amide local anaesthetics. Local anaesthetics block the initiation and propagation of action potentials by preventing the voltage-dependent increase in Na⁺ conductance. They can be used for surgical anesthesia and postoperative pain management. For surgical anesthesia, bupivacaine has been approved for epidural use, peripheral neural blockade, and local infiltration as well as for pain management. Typically, a 0.75% solution of bupivacaine is administered for ophthalmic surgery. A 0.5% bupivacaine solution may be administered for Cesarean section or peripheral nerve block. A 0.25% solution of bupivacaine may be administered in infiltration anaesthesia or to women in early labor requesting epidural analgesia. A composition of 0.125% bupivacaine may be used for postoperative pain management. Levobupivacaine and ropivacaine have similar administration, while mepivacaine is ineffective as a topical anaesthetic.

Compounds of Formula (XXX)

Compounds of formula (XXX) have the formula:

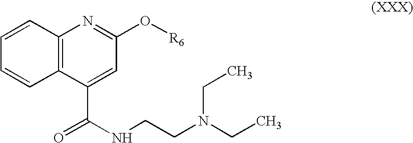

wherein R₆ is —((CH)₂)₂OCH₃, —((CH)₂)₂OCH₂CH₃, or —((CH)₂)₃CH₃. An exemplary member of this class is dibucaine (2-butoxy-N-(2-(diethylamino)ethyl)cinchoninamide), which has the formula (XXXI):

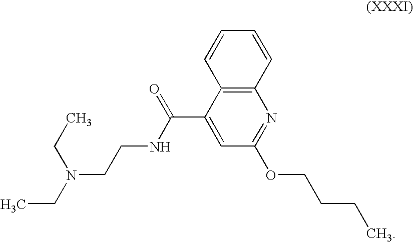

Dibucaine (2-butoxy-N-(2-(diethylamino)ethyl)cinchoninamide) is used as a topical analgesic, anaesthetic and antipruritic for the temporary relief of pain and itching due to minor burns, sunburn, minor cuts, abrasions, insect bites and minor skin irritations. It is typically formulated as a 0.5% to 1% solution.

Vinca Alkaloids—Compounds of Formula (XXXII)

“Vinca alkaloid” refers to a compound of formula (XXXII), which encompasses plant-derived antiproliferative compound such as vinblastine, vinleurosine, vinrosidine or vincristine (each found in the Madagascar periwinkle, Catharanthus roseus) as well as the semi-synthetic derivatives such as vindesine and vinorelbine. They are antineoplastic agents that act by binding tubulin and inhibiting its polymerization into microtubules.

Examples of vinca alkaloids are vinblastine, vinorelbine, vindesine, and vincristine.

Compounds of formula (XXXII) have the formula:

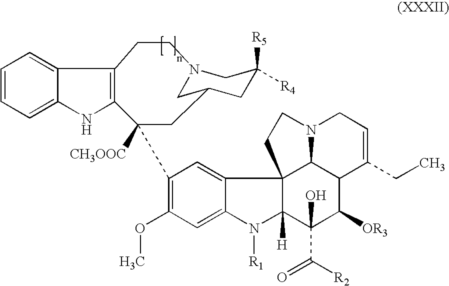

wherein R₁ is CHO, CH₃, or H, R₂ is OCH₃ or NH₂, R₃ is OCOCH₃ or OH, R₄ is H, CH₃, CH₂CH₃, or CF₂CH₃, R₅ is H, OH, or CH₂CH₃, and n=0 or 1.

Antiproliferative Agents

Antiproliferative agents are described above. They include, but are not limited to microtubule inhibitors, topoisomerase inhibitors, platins, alkylating agents, and anti-metabolites. Exemplary antiproliferative agents useful in the present application include paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine. Additional antiproliferative agents may be found in Table 4.

Exemplary Drug Combinations

In certain embodiments, the drug combinations of the present invention may comprise (1) a first compound selected from bupivacaine, levobupivacaine, ropivacaine, and mepivacaine, and (2) a second compound selected from vinblastine, vincristine, vindestine, or vinorelbine.

In certain other embodiments, the drug combinations of the present invention may comprise dibucaine and a second compound selected from vinblastine, vincristine, vindestine, or vinorelbine.

In certain embodiments, the drug combinations of the present invention may comprise bupivacaine and vinblastine, levobupivacaine and vinblastine, dibucaine and vinblastine, mepivacaine and vinblastine, ropivacaine and vinblastine.

In certain embodiment, the drug combinations of the present invention may comprise levobupivicaine and vinorelbine, or dibucaine and vinorelbine.

Combinations Comprising Pentamidine and Antiproliferative Agents

In certain embodiments, the drug combinations according to the present invention may comprise pentamidine (or its structural or functional analogs, salts, or metabolites) and an antiproliferative agent.

Pentamidine, Analogs, Salts, and Metabolites

Pentamidine, its analogs, pharmaceutically active or acceptable salts and metabolites are described as above in the section related to combinations comprising chlorpromazine and pentamidine.

In certain embodiments, pentamidine analogs have formula (XXXIII) 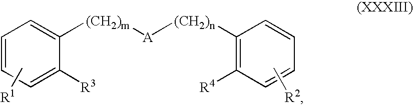
or a pharmaceutically acceptable salt thereof,

wherein A is

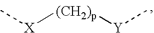

each of X and Y is, independently, O or NH,

p is an integer between 2 and 6, inclusive,

each of m and n is, independently, an integer between 0 and 2, inclusive, wherein the sum of m and n is greater than 0,

each of R¹ and R² is, independently, selected from the group represented by

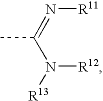

wherein R¹² is H, C₁-C₆ alkyl, C₁-C₈ cycloalkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, or, R¹³ is H, C₁-C₆ alkyl, C₁-C₈ cycloalkyl, C₆-C₁₈ aryloxy C₁-C₆ alkyl, C₁-C₆ alkoxy C₁-C₆ alkyl, hydroxy C₁-C₆ alkyl, C₁-C₆ alkylamino C₁-C₆ alkyl, amino C₁-C₆ alkyl, carbo(C₁-C₆ alkoxy), carbo(C₆-C₁₈ aryl-C₁-C₆ alkoxy), carbo(C₆-C₁₈ aryloxy), or C₆-C₁₈ aryl, and R¹¹ is H, OH, or oxy(C₁-C₆ alkyl), or R¹¹ and R¹² together represent

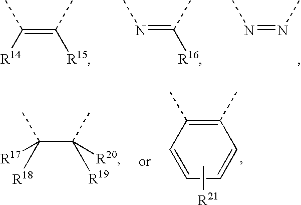

wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl,

each of R³ and R⁴ is, independently, H, Cl, Br, OH, OCH₃, OCF₃, NO₂, and NH₂, or R³ and R⁴ together form a single bond.

In certain embodiments, A is

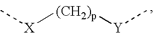

each of X and Y is, independently, O or NH,

p is an integer between 2 and 6, inclusive,

each of m and n is 0, and

each of R¹ and R² is, independently, selected from the group represented by

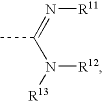

wherein R¹² is C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent

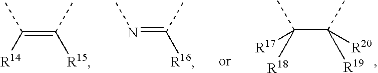

wherein each of R¹⁴, R¹⁵, and R¹⁶ is, independently, H, C₁-C₆ alkyl, halogen, or trifluoromethyl, each of R¹⁷, R¹⁸, and R¹⁹ is, independently, H or C₁-C₆ alkyl, and R²⁰ is C₁-C₆ alkyl, C₁-C₆ alkyloxy, or trifluoromethyl.

In certain embodiments, A is

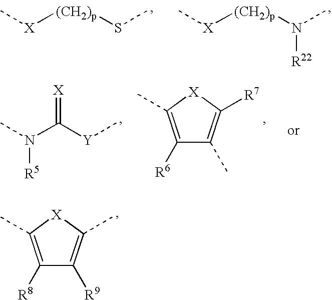

each of X and Y is, independently, O, NR¹⁰, or S,

each of R⁵ and R¹⁰ is, independently, H or C₁-C₆ alkyl,

each of R⁶, R⁷, R⁸, and R⁹ is, independently, H, C₁-C₆ alkyl, halogen, C₁-C₆ alkyloxy, C₆-C₁₈ aryloxy, or C₆-C₁₈ aryl C₁-C₆ alkyloxy,

R²² is C₁-C₆ alkyl,

p is an integer between 2 and 6, inclusive,

each of m and n is, independently, an integer between 0 and 2, inclusive,

each of R¹ and R² is, independently, selected from the group represented by

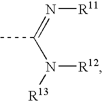

wherein R¹² is H, C₁-C₆ alkyl, C₁-C₈ cycloalkyl, C₁-C₆ alkoxy C₁-C₆ alkyl, hydroxy C₁-C₆ alkyl, C₁-C₆ alkylamino C₁-C₆ alkyl, amino C₁-C₆ alkyl, or C₆-C₁₈ aryl, R¹³ is H, C₁-C₆ alkyl, C₁-C₈ cycloalkyl, C₆-C₁₈ aryloxy C₁-C₆ alkyl, C₁-C₆ alkyloxy C₁-C₆ alkyl, hydroxy C₁-C₆ alkyl, C₁-C₆ alkylamino C₁-C₆ alkyl, amino C₁-C₆ alkyl, carbo(C₁-C₆ alkyloxy), carbo(C₆-C₁₈ aryl C₁-C₆ alkyloxy), carbo(C₆-C₁₈ aryloxy), or C₆-C₁₈ aryl, and R¹¹ is H, OH, or C₁-C₆ alkyloxy, or R¹¹ and R¹² together represent

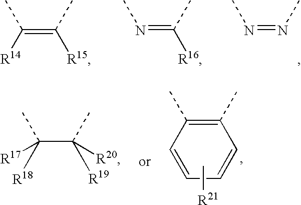

wherein each of R¹⁴, R¹⁵, and R¹⁶ is, independently, H, C₁-C₆ alkyl, halogen, or trifluoromethyl, each of R¹⁷, R¹⁸, R¹⁹, and R²⁰ are, independently, H or C₁-C₆ alkyl, and R²¹ is H, halogen, trifluoromethyl, OCF₃, NO₂, C₁-C₆ alkyl, C₁-C₈ cycloalkyl, C₁-C₆ alkyloxy, C₁-C₆ alkyloxy C₁-C₆ alkyl, hydroxy C₁-C₆ alkyl, C₁-C₆ alkylamino C₁-C₆ alkyl, amino C₁-C₆ alkyl, or C₆-C₁₈ aryl, and

each of R³ and R⁴ is, independently, H, Cl, Br, OH, OCH₃, OCF₃, NO₂, and NH₂, or R³ and R⁴ together form a single bond.

Antiproliferative Agents

Antiproliferative agents useful in combination with pentamidine include both Group A antiproliferative agents and Group B antiproliferative agents.

“Group A antiproliferative agent” refers to any antiproliferative agent that is not a Group B antiproliferative agent.

Examples of Group A agents are those listed in Table 4. Group A antiproliferative agents of the invention also include those alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors that are not Group B antiproliferative agents, as defined herein (see Table 6).

In certain embodiments, the Group A antiproliferative agent is vinblastine, carboplatin, etoposide, or gemcitabine.

“Group B antiproliferative agent” refers to any antiproliferative agent selected from the group of compounds in Table 6.

TABLE 6
(Group B)

melphalan
carmustine
cisplatin
5-fluorouracil
mitomycin C
adriamycin (doxorubicin)
bleomycin
Paclitaxel (Taxol ®)

Exemplary Drug Combinations

In one embodiment, the combinations of the present invention comprises (1) a compound of formula (XXXIII) selected from pentamidine, propamidine, butamidine, heptamidine, nonamidine, dibrompropamidine, 1,3-bis(4-amidino-2-methoxyphenoxy)propane, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[4-(2-imidazolinyl)phenyl]-3-methoxyfuran, 2,5-bis[4-(N-isopropylamidino)phenyl]-3-methylfuran, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, 2,5-bis[4-(N-(3-fluoro)phenoxycarbonyl)amidinophenyl]furan, and pharmaceutically active or acceptable salts of the above listed agents, and (2) an antiproliferative agent selected from vinblastine, carboplatin, adriamycin (doxorubicin), etoposide, and gemcitabine.

In certain embodiments, the drug combinations comprise (1) a compound selected from pentamidine, propamidine, butamidine, heptamidine, nonamidine, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, and pharmaceutically active or acceptable salts thereof, and (2) an antiproliferative agent selected from vinblastine, carboplatin, adriamycin (doxorubicin), etoposide, and gemcitabine.

In certain embodiments, the drug combinations comprise (1) an endo-exonuclease inhibitor and (2) one or more Group A antiproliferative agents (e.g., vinblastine, carboplatin, etoposide, and gemcitabine).

In certain embodiments, the drug combinations comprise (1) a phosphatase of regenerating liver (PRL) inhibitor or a PTB1B inhibitor and (2) one or more Group A antiproliferative agents (e.g., vinblastine, carboplatin, etoposide, or gemcitabine).

In certain embodiments, the drug combinations comprise pentamidine and vinblastine, pentamidine and carboplatin, pentamidine and doxorubicin, pentamidine and etoposide, pentamidine and gemcitabine, or pentamidine and 5-fluorouracil.

Combinations Comprising Triazoles and Antiarrhythmic Agents

In certain embodiments, the drug combinations according to the present invention may comprise triazoles (or their structural or functional analogs, pharmaceutically active or acceptable salts, or metabolites) and antiarrhythmic agents (or their structural or functional analogs, pharmaceutically active or acceptable salts, or metabolites). In certain embodiments, the drug combinations may further comprise one or more antiproliferative agents.

Antiarrhythmic Agents

“Antiarrhythmic agent” refers to a drug that reduces cardiac arrhythmia. Examples of antiarrhythmic agents are drugs that block voltage-sensitive sodium channels, beta-adrenoceptor antagonists, drugs that prolong the cardiac action potential, and Ca²⁺ channel antagonists.

Generally, there is little structure-activity relationship between antiarrhythmic agents with regard to their antiarrhythmic effects. By the Vaughan Williams' classification, antiarrhythmic agents are generally divided into four classes.

Class I drugs block voltage-sensitive sodium channels. Class I drugs are further divided into Classes IA, IB and IC. Class IA drugs lengthen the duration of the myocardial action potential while decreasing the maximal rate of depolarization. Class IA drugs include hydroxyl quinidine, quinidine, disopyramide, and procainamide. Class IB antiarrhythmic agents decrease the maximal rate of depolarization as well as decreasing the duration of the myocardial action potential. Examples of Class IB agents are lidocaine, tocainide, mexiletine, and phenyloin. Class IC antiarrhythmic agents decrease the maximal rate of depolarization while having no effect on the duration of the myocardial action potential. Examples include flecainide and encainide.

Class II drugs are beta-adrenoceptor antagonists, examples of which are propranolol, acebutolol, esmolol, and sotalol.

Class III drugs prolong the cardiac action potential, thereby increasing the refractory period suppressing the ectopic and re-entrant activity, such as amiodarone, sotalol, and bretylium tosylate.

Class IV drugs are Ca²⁺ channel antagonists, which block the slow inward current that is carried by calcium ions during the myocardial action potential. Examples of Class IV drugs are nifedipine, amlodipine, felodipine, flunarizine, isradipine, nicardipine, diltiazem, verapamil, and bepridil.

Other antiarrhythmic agents that do not fall within one of the above categories but are considered antiarrhythmic agents include digoxin and adenosine.

Amiodarone

Amiodarone (2-Butyl-3-benzofuranyl)(4-(2-(diethylamino)ethoxy)-3,5-diidophenyl)methanone; Cordarone™) has the following structure:

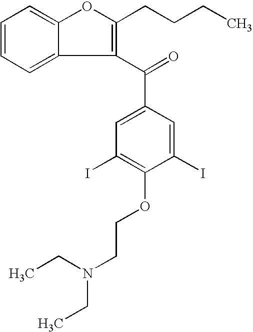

Related compounds to amiodarone include di-N-desethylamiodarone, desethylamiodarone, desoxoamiodarone, etabenzarone, and 2-butylbenzofuran-3-yl, 4 hydroxy-3,5-diiodophenyl ketone.

Bepridil

Bepridil (beta-((2-methylpropoxy)methyl)-N-phenyl-N-(phenylmethyl)-1-pyrrolidineethanamine) has the following structure:

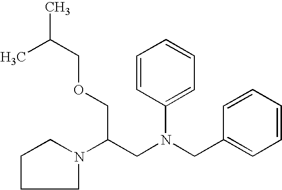

Nicardipine

Nicardipine (2-(benzyl-methyl amino)ethyl methyl 1,4-dihydro-2,6-dimethyl-4-(m-nitrophenyl)-3,5-pyridinedicarboxylate monohydrochloride) is a class IV antiarrhythmic having the following structure:

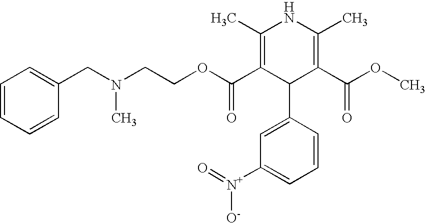

Additional antiarrhythmic agents include amlodipine, nifedipine, diltiazem, felodipine, flunarizine, isradipine, nimodipine, and verapamil.

Triazoles

“Triazole” refers to a compound having a five-membered ring of two carbon atoms and three nitrogen atoms. Triazoles useful in the present invention may have formula (XXXIV):

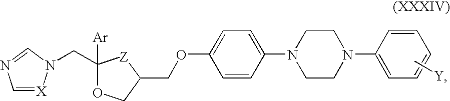

or a pharmaceutically active or acceptable salt thereof, wherein X is CH₂ or N; Z is CH₂ or O; Ar is selected from the group consisting of phenyl, thienyl, halothienyl, and substituted phenyl having from 1 to 3 substitutents, each independently selected from the group consisting of halo, C₁-C₆ linear or branched alkyl, linear or branched C₁-C₆ alkoxy, and trifluoromethyl; and Y is a group having the formula:

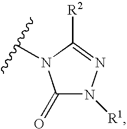

wherein R¹ is selected from the group consisting of C₁-C₆ linear or branched alkyl having 0 or 1 hydroxyl substitutents and C₁-C₆ linear or branched alkaryl, and R² is selected from the group consisting of H, linear or branched C₁-C₆ alkyl, and C₁-C₆ alkaryl, wherein said aryl group is a phenyl ring having from 0 to 3 substitutents, each independently selected from the group consisting of halo, C₁-C₆ linear or branched alkyl, linear or branched C₁-C₆ alkoxy, and trifluoromethyl. Exemplary triazoles of formula (XXXIV) include itraconazole, hydroxyitraconazole, posaconazole, and saperconazole.

Antiproliferative Agents

Antiproliferative agents are described above. Exemplary antiproliferative agents include cisplatin, daunorubicin, doxorubicin, etoposide, methotrexate, mercaptopurine, 5-fluorouracil, hydroxyurea, vinblastine, vincristine, paclitaxel, bicalutamide, bleomycin, carboplatin, carmustine, cyclophosphamide, docetaxel, epirubicin, gemcitabine hcl, goserelin acetate, imatinib, interferon alpha, irinotecan, lomustine, leuprolide acetate, mitomycin, rituximab, tamoxifen, trastuzumab, or any combination thereof.

Exemplary Drug Combinations

In certain embodiments, the drug combinations according to the present invention comprise (1) an antiarrhythmic agent selected from amiodarone, di-N-desethylamiodarone, desethylamiodarone, bepridil, and nicardipine, and (2) a triazole selected from itraconazole, hydroxyitraconazole, posaconazole, and saperconazole.

In certain embodiments, the drug combinations comprise itraconazole and amiodarone, bepridil and itraconazole, or itraconazole and nicardipine.

Combinations Comprising Azoles and HMG-CoA Reductase Inhibitors

In certain embodiments, the drug combinations according to the present invention may comprise azoles (or their structural or functional analogs, pharmaceutically active or acceptable salts, or metabolites) and HMG-CoA reductase inhibitors (or their structural or functional analogs, pharmaceutically active or acceptable salts, or metabolites). In certain embodiments, the drug combinations may further comprise one or more antiproliferative agents.

HMG-CoA Reductase Inhibitors

“HMG-CoA reductase inhibitor” refers to a compound that inhibits the enzymatic activity of 3-hydroxy-3-methylglutaryl-coenzyme A (HMG-CoA) reductase by at least about 10%. HMG-CoA reductase inhibitors include but are not limited to simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, and pitavastatin, as well as pharmaceutically active or acceptable salts thereof (e.g., simvastatin sodium, lovastatin sodium, fluvastatin sodium, etc.).

Additional HMG-CoA reductase inhibitors and analogs thereof useful in the methods and compositions of the present invention are described in U.S. Pat. Nos. 3,983,140; 4,231,938; 4,282,155; 4,293,496; 4,294,926; 4,319,039; 4,343,814; 4,346,227; 4,351,844; 4,361,515; 4,376,863; 4,444,784; 4,448,784; 4,448,979; 4,450,171; 4,503,072; 4,517,373; 4,661,483; 4,668,699; 4,681,893; 4,719,229; 4,738,982; 4,739,073; 4,766,145; 4,782,084; 4,804,770; 4,841,074; 4,847,306; 4,857,546; 4,857,547; 4,940,727; 4,946,864; 5,001,148; 5,006,530; 5,075,311; 5,112,857; 5,116,870; 5,120,848; 5,166,364; 5,173,487; 5,177,080; 5,273,995; 5,276,021; 5,369,123; 5,385,932; 5,502,199; 5,763,414; 5,877,208; and 6,541,511; and U.S. Patent Application Publication Nos. 2002/0013334 A1; 2002/0028826 A1; 2002/0061901 A1; and 2002/0094977 A1.

Azoles

“Azole” refers to any member of the class of anti-fungal compounds having a five-membered ring of three carbon atoms and two nitrogen atoms (e.g., imidazoles) or two carbon atoms and three nitrogen atoms (e.g., triazoles), which are capable of inhibiting fungal growth. A compound is considered “anti-fungal” if it inhibits growth of a species of fungus in vitro by at least 25%.

Azoles that can be employed in the methods and compositions of the invention include fluconazole, itraconazole, hydroxyitraconazole, posaconazole, saperconazole, ketoconazole, clotrimazole, terconazole, econazole, tioconazole, oxiconazole, butoconazole, and miconazole.

Additional azoles and analogs thereof useful in the methods and compositions of the present invention are described in U.S. Pat. Nos. 3,575,999; 3,705,172; 3,717,655; 3,936,470; 4,062,966; 4,078,071; 4,107,314; 4,124,767; 4,144,346; 4,223,036; 4,229,581; 4,232,034; 4,244,964; 4,248,881; 4,267,179; 4,272,545; 4,307,105; 4,335,125; 4,360,526; 4,368,200; 4,402,968; 4,404,216; 4,416,682; 4,458,079; 4,466,974; 4,483,865; 4,490,530; 4,490,540; 4,503,055; 4,510,148; 4,554,286; 4,619,931; 4,625,036; 4,628,104; 4,632,933; 4,661,602; 4,684,392; 4,735,942; 4,761,483; 4,771,065; 4,789,587; 4,818,758; 4,833,141; 4,877,878; 4,916,134; 4,921,870; 4,960,782; 4,992,454; and 5,661,151.

Antiproliferative Agents

Antiproliferative agents are described above. Exemplary antiproliferative agents include cisplatin, daunorubicin, doxorubicin, etoposide, methotrexate, mercaptopurine, 5-fluorouracil, hydroxyurea, vinblastine, vincristine, paclitaxel, or any combination thereof.

Exemplary Drug Combinations

In certain embodiments, the drug combinations of the present invention comprise (1) an azole selected from fluconazole, itraconazole, hydroxyitraconazole, posaconazole, saperconazole, ketoconazole, clotrimazole, terconazole, econazole, tioconazole, oxiconazole, butoconazole, miconazole, and pharmaceutically active or acceptable salts thereof, and (2) an HMG-CoA reductase inhibitor selected from simvastatin, lovastatin, mevastatin, pravastatin, monacolin M, monacolin X, fluvastatin, atorvastatin, cerivastatin, rosuvastatin, fluindostatin, velostatin, compactin, dihydrocompactin, rivastatin, dalvastatin, pitavastatin, and pharmaceutically active or acceptable salts thereof.

In certain embodiments, the drug combinations of the present invention may comprise simvastatin and itraconazole, atorvastatin and itraconazole, fluvastatin and itraconazole, lovastatin and itraconazole, atorvastatin and clotrimazole, atorvastatin and econazole, atorvastatin and ketoconazole, lovastatin and econazole, atorvastatin and terconazole, cerivastatin and itraconazole; or lovastatin and tioconazole.

Combinations Comprising Phenothiazine Conjugates or Phenothiazines and Antiproliferative Agents

In certain embodiments, the drug combinations of the present invention may comprise or be phenothiazine conjugates (e.g., conjugates comprising phenothiazines and antiproliferative agents). The phenothiazine conjugates generally have three characteristic components: a phenothiazine covalently tethered, via a linker, to a group that is bulky or charged.

In certain embodiments, the drug combination may comprise phenothiazines and antiproliferative agents.

Phenothiazine Conjugates

Phenothiazines

By “phenothiazine” is meant any compound having a phenothiazine ring structure or related ring structure as shown below. Thus, ring systems for which the ring sulfur atom is oxidized, or replaced by O, NH, CH₂, or CH═CH are encompassed by the generic description “phenothiazine.” For all of the ring systems show below, phenothiazines include those ring substitutions and nitrogen substitutions provide for in formulas ((VI)(A)) and (VII).

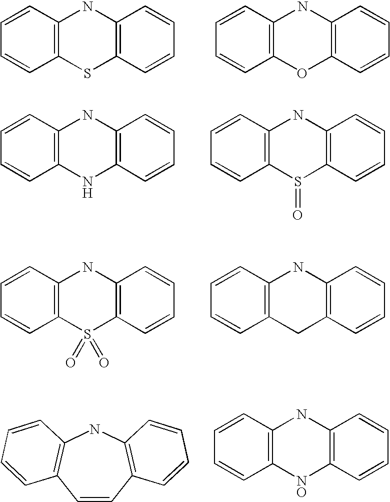

By “parent phenothiazine” is meant the phenothiazine which is modified by conjugation to a bulky group or a charged group. A phenothiazine conjugate includes a phenothiazine covalently attached via a linker to a bulky group of greater than 200 daltons or a charged group of less than 200 daltons.

In certain embodiments, the phenothiazine conjugate is described by formula (VII):

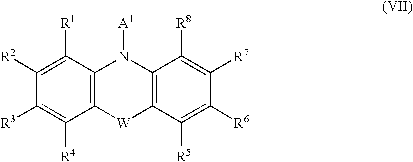

In formula (VII), R² is selected from the group consisting of: CF₃, halogen, OCH₃, COCH₃, CN, OCF₃, COCH₂CH₃, CO(CH₂)₂CH₃, S(O)₂CH₃, S(O)₂N(CH₃)₂, and SCH₂CH₃; A¹ is selected from the group consisting of G¹,

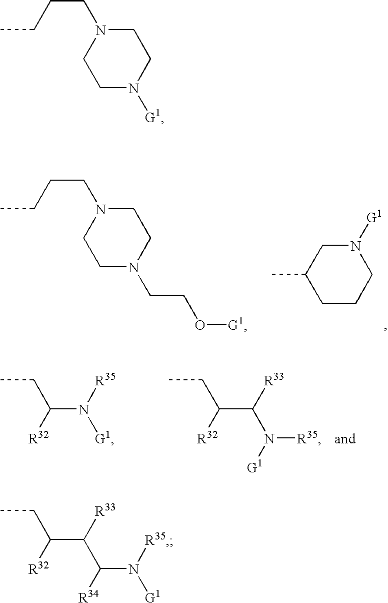

each of R¹, R³, R⁴, R⁵, R⁶, R⁷, and R⁸ is independently H, OH, F, OCF₃, or OCH₃; R³², R³³, R³⁴, and R³⁵, are each, independently, selected from H or C₁₋₆ alkyl; W is selected from the group consisting of: NO,

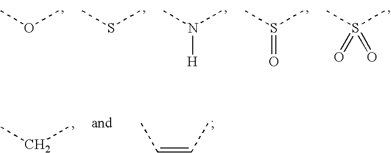

and G¹ is a bond between the phenothiazine and the linker.

Phenothiazines useful in the drug combinations include compounds having a structure as shown in formula (VI)(A): 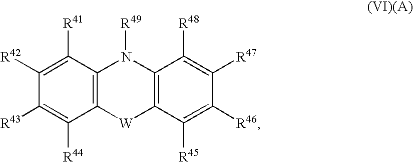
or a pharmaceutically acceptable salt thereof, wherein R⁴² is selected from the group consisting of: CF₃, halogen, OCH₃, COCH₃, CN, OCF₃, COCH₂CH₃, CO(CH₂)₂CH₃, S(O)₂CH₃, S(O)₂N(CH₃)₂, and SCH₂CH₃;
R⁴⁹ is selected from the group consisting of: 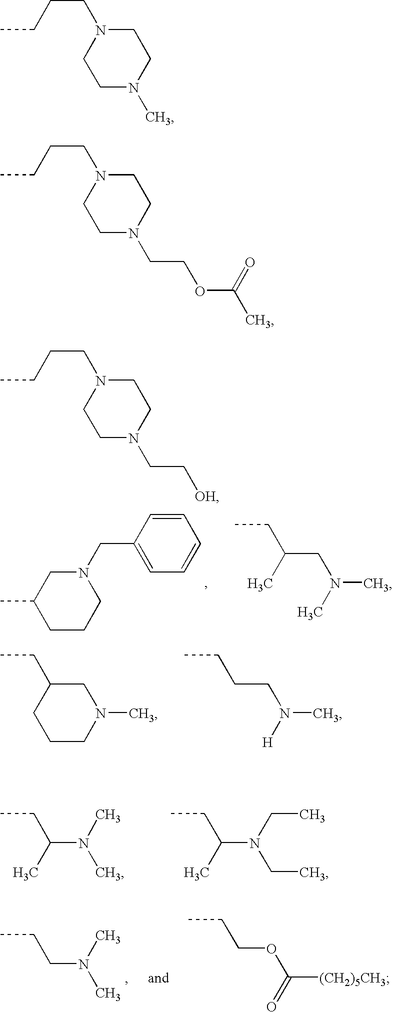
each of R⁴¹, R⁴³, R⁴⁴, R⁴⁵, R⁴⁶, R⁴⁷, and R⁴⁸ is independently H, OH, F, OCF₃, or OCH₃; and W is selected from the group consisting of: NO,

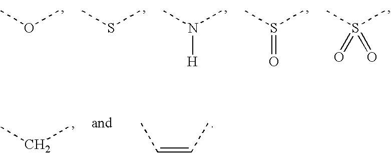

Phenothiazines useful in the present invention include, without limitation, acepromazine, cyamemazine, fluphenazine, mepazine, methotrimeprazine, methoxypromazine, perazine, pericyazine, perimethazine, perphenazine, pipamazine, pipazethate, piperacetazine, pipotiazine, prochlorperazine, promethazine, propionylpromazine, propiomazine, sulforidazine, thiazinaminiumsalt, thiethylperazine, thiopropazate, thioridazine, trifluoperazine, trimeprazine, thioproperazine, trifluomeprazine, triflupromazine, chlorpromazine, chlorproethazine, those compounds in PCT application WO02/057244, and those compounds in U.S. Pat. Nos. 2,415,363; 2,519,886; 2,530,451; 2,607,773; 2,645,640; 2,766,235; 2,769,002; 2,784,185; 2,785,160; 2,837,518; 2,860,138; 2,877,224; 2,921,069; 2,957,870; 2,989,529; 3,058,979; 3,075,976; 3,194,733; 3,350,268; 3,875,156; 3,879,551; 3,959,268; 3,966,930; 3,998,820; 4,785,095; 4,514,395; 4,985,559; 5,034,019; 5,157,118; 5,178,784; 5,550,143; 5,595,989; 5,654,323; 5,688,788; 5,693,649; 5,712,292; 5,721,254; 5,795,888; 5,597,819; 6,043,239; and 6,569,849, each of which is incorporated herein by reference. Structurally related phenothiazines having similar antiproliferative properties are also intended to be encompassed by this group, which includes any compound of formula ((VI)(A)), described above.

The structures of several of the above-mentioned phenothiazines are provided below. Phenothiazine conjugates of the invention are prepared by modification of an available functional group present in the parent phenothiazine. Alternatively, the substitutent at the ring nitrogen can be removed from the parent phenothiazine prior to conjugation with a bulky group or a charged group.

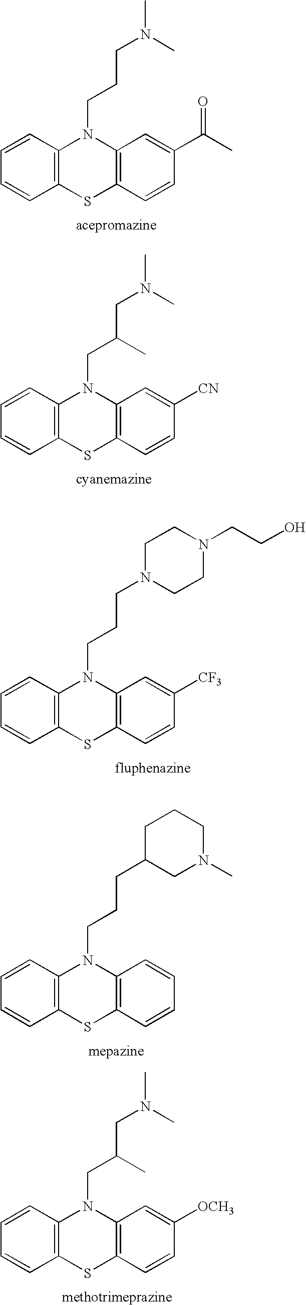

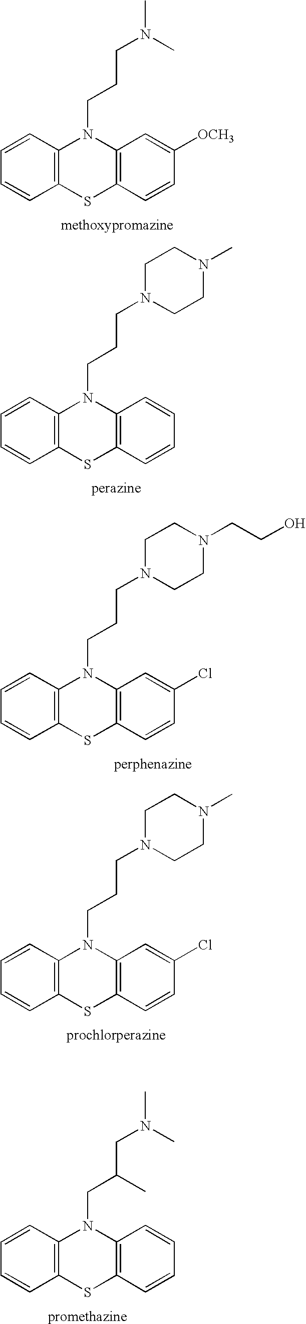

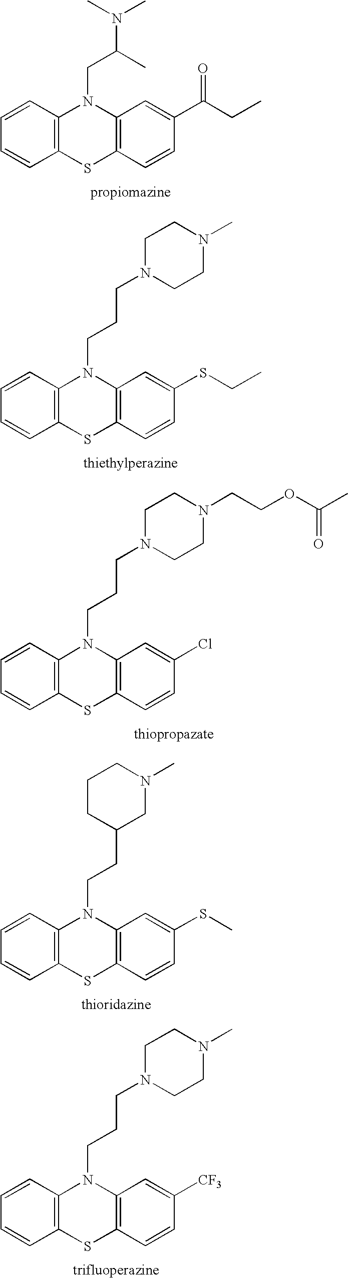

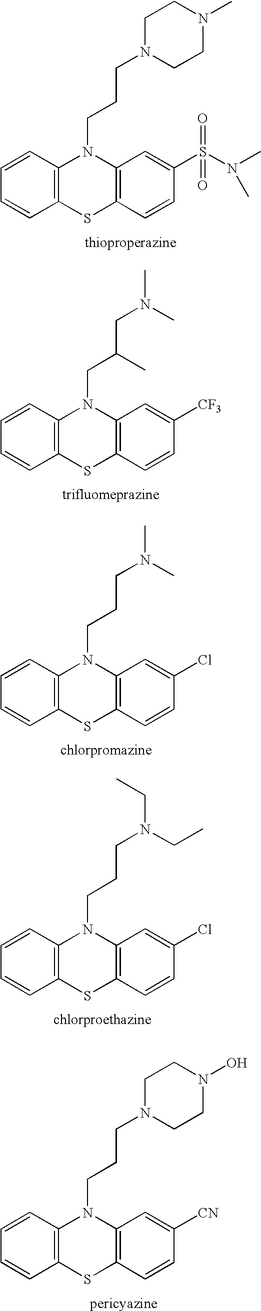

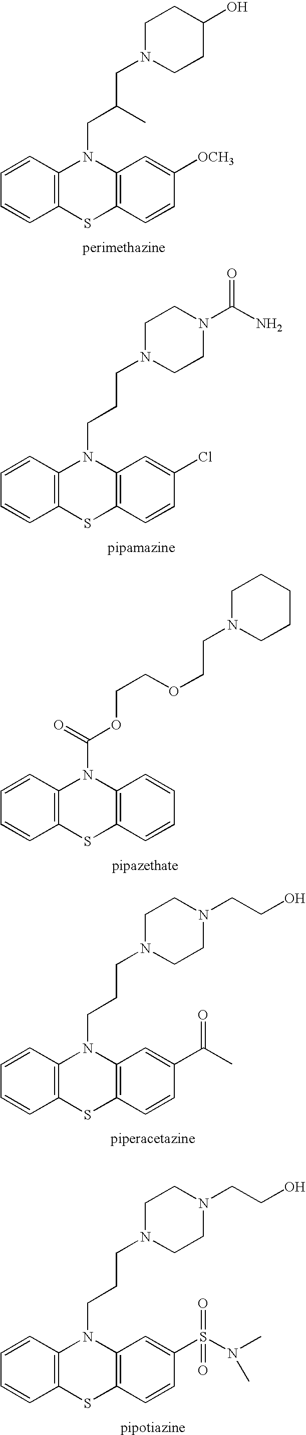

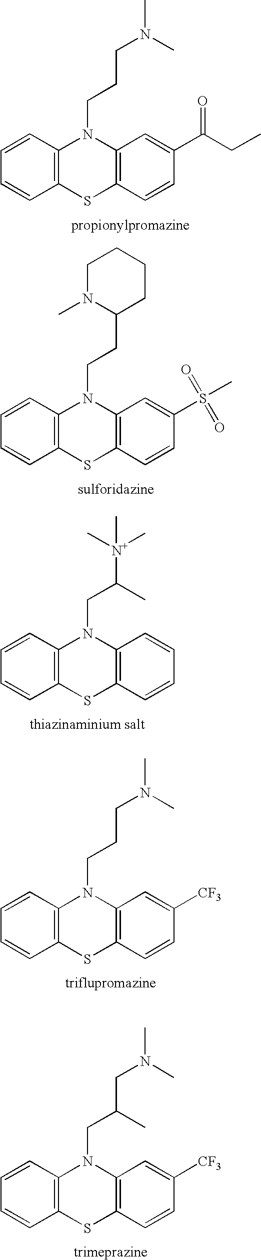

Phenothiazine compounds can be prepared using, for example, the synthetic techniques described in U.S. Pat. Nos. 2,415,363; 2,519,886; 2,530,451; 2,607,773; 2,645,640; 2,766,235; 2,769,002; 2,784,185; 2,785,160; 2,837,518; 2,860,138; 2,877,224; 2,921,069; 2,957,870; 2,989,529; 3,058,979; 3,075,976; 3,194,733; 3,350,268; 3,875,156; 3,879,551; 3,959,268; 3,966,930; 3,998,820; 4,785,095; 4,514,395; 4,985,559; 5,034,019; 5,157,118; 5,178,784; 5,550,143; 5,595,989; 5,654,323; 5,688,788; 5,693,649; 5,712,292; 5,721,254; 5,795,888; 5,597,819; 6,043,239; and 6,569,849, each of which is incorporated herein by reference.

Linkers

The linker component of the invention is, at its simplest, a bond between a phenothiazine and a group that is bulky or charged. The linker provides a linear, cyclic, or branched molecular skeleton having pendant groups covalently linking a phenothiazine to a group that is bulky or charged.

Thus, the linking of a phenothiazine to a group that is bulky or charged is achieved by covalent means, involving bond formation with one or more functional groups located on the phenothiazine and the bulky or charged group. Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of a phenothiazine and a group that is bulky or charged may be effected using a linker that contains reactive moieties capable of reaction with such functional groups present in the phenothiazine and the bulky or charged group. For example, a hydroxyl group of the phenothiazine may react with a carboxyl group of the linker, or an activated derivative thereof, resulting in the formation of an ester linking the two.

Examples of moieties capable of reaction with sulfhydryl groups include α-haloacetyl compounds of the type XCH₂CO— (where X=Br, Cl or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulphide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X=Cl, Br or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sulfhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g. wherein the azide group is generated from a preformed hydrazide derivative using sodium nitrite, as described by Wetz et al., Anal Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in the phenothiazine and/or the bulky or charged group may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of the phenothiazine with a reactive chemical group of the bulky or charged group without introducing additional linking material may, if desired, be used in accordance with the invention. For example, the ring nitrogen of the phenothiazine can be linked directly via an amide bond to the charged or bulky group.

Most commonly, however, the linker will include two or more reactive moieties, as described above, connected by a spacer element. The presence of such a spacer permits bifunctional linkers to react with specific functional groups within the phenothiazine and the bulky or charged group, resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between the phenothiazine and the bulky or charged group.

Spacer elements in the linker typically consist of linear or branched chains and may include a C₁₋₁₀ alkyl, a heteroalkyl of 1 to 10 atoms, a C₂₋₁₀ alkene, a C₂₋₁₀ alkyne, C₅₋₁₀ aryl, a cyclic system of 3 to 10 atoms, or —(CH₂CH₂O)ₙCH₂CH₂—, in which n is 1 to 4.

In some instances, the linker is described by formula (XXXV):
G¹-(Z¹)ₒ-(Y¹)ᵤ-(Z²)ₛ-(R⁹)-(Z³)ₜ-(Y²)ᵥ-(Z⁴)ₚ-G² (XXXV)

In formula (XXXV), G¹ is a bond between the phenothiazine and the linker, G² is a bond between the linker and the bulky group or between the linker and the charged group, each of Z¹, Z², Z³, and Z⁴ is, independently, selected from O, S, and NR³⁹; R³⁹ is hydrogen or a C₁₋₁₀ alkyl group; each of Y¹ and Y² is, independently, selected from carbonyl, thiocarbonyl, sulphonyl, phosphoryl or similar acid-forming groups; o, p, s, t, u, and v are each independently 0 or 1; and R⁹ is C₁₋₁₀ alkyl, C₁₋₁₀ heteroalkyl, C₂₋₁₀ alkenyl, a C₂₋₁₀ alkynyl, C₅₋₁₀ aryl, a cyclic system of 3 to 10 atoms, or a chemical bond linking G¹-(Z¹)ₒ-(Y¹)ᵤ-(Z²)ₛ- to -(Z³)ₜ-(Y²)ᵥ-(Z⁴)ₚ-G².

Bulky Groups

In certain embodiments, bulky groups have a molecular weight greater than 200, 300, 400, 500, 600, 700, 800, 900, or 1000 daltons. In certain embodiments, these groups are attached through the ring nitrogen of the phenothiazine.

By “linked through the ring nitrogen” is meant that the charged group, bulky group, or linker is covalently attached to a substitutent of ring nitrogen as identified below.

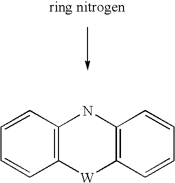

In certain embodiments, the bulky group comprises a naturally occurring polymer, such as a glycoprotein, a polypeptide (alpha-1-acid glycoprotein), or a polysaccharide (e.g., hyaluronic acid). In certain other embodiments, the bulky group comprises a synthetic polymer, such as a polyethylene glycol or N-hxg.

In certain embodiments, a bulky group is a charged bulky group, such as the polyguanidine peptoid (N-hxg)₉, shown below. Each of the nine guanidine side chains is a charged guanidinium cation at physiological pH.

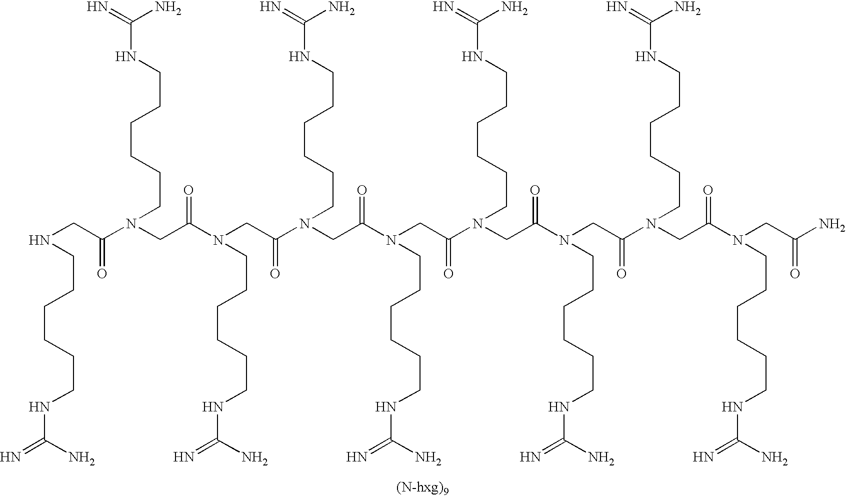

Additional charged bulky group include, without limitation, charged polypeptides, such as poly-arginine (guanidinium side chain), poly-lysine (ammonium side chain), poly-aspartic acid (carboxylate side chain), poly-glutamic acid (carboxylate side chain), or poly-histidine (imidazolium side chain).

In certain embodiments, a charged polysaccharide (e.g., hyaluronic acid as shown below) may also be used.

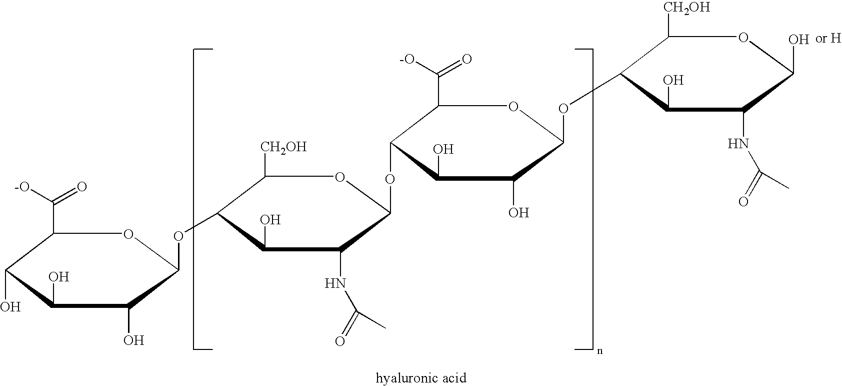

The bulky group can be an antiproliferative agent used in the combinations of the invention. Such conjugates are desirable where the two agents have matching pharmacokinetic profiles to enhance efficacy and/or to simplify the dosing regimen.

The bulky group may also include another therapeutic agent. Desirably, the therapeutic agent conjugated to the phenothiazine of formula (VII) via a linker of formula (XXXV) is a compound of formula (XXXVI):

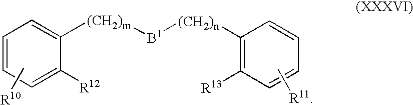

In formula (XXXVI), B¹ is selected from

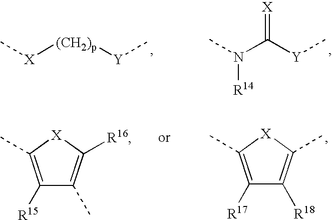

wherein each of X and Y is, independently, O, NR¹⁹, or S; each of R¹⁴ and R¹⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R¹⁵, R¹⁶, R¹⁷, and R¹⁸ is, independently, H, halogen, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; p is an integer between 2 and 6, inclusive; each of m and n is, independently, an integer between 0 and 2, inclusive; each of R¹⁰ and R¹¹ is

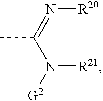

wherein R²¹ is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; R²⁰ is H, OH, or acyl, or R²⁰ and R²¹ together represent

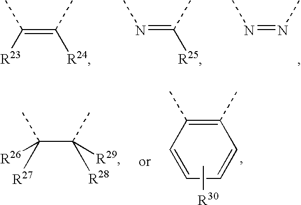

wherein each of R²³, R²⁴, and R²⁵ is, independently, H, halogen, trifluoromethyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R²⁶, R²⁷, R²⁸, and R²⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁰ is H, halogen, trifluoromethyl, OCF₃, NO₂, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R¹² and R¹³ is, independently, H, Cl, Br, OH, OCH₃, OCF₃, NO₂, and NH₂, or R¹² and R¹³ together form a single bond; and G² is a bond between the compound of formula (XXXVI) and the linker.

Antiproliferatives that can be conjugates to a phenothiazine compound include pentamidine, shown below, as well as 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorene, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, or 2,5-bis[4-(N-(3-fluoro)phenoxycarbonyl)amidinophenyl]furan.

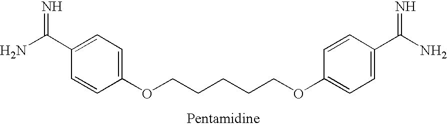

Methods for making any of the foregoing compounds are described in U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, an U.S. Patent Application Publication Nos. US 2001/0044468 A1 and US 2002/0019437 A1.

The conjugate comprising, for example, a phenothiazine (A) and pentamidine (B), can be linked, without limitation, as dimers, trimers, or tetramers, as shown below. 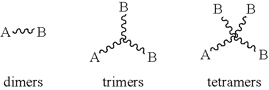
Charged Groups

By “charged group” is meant a group comprising three or more charged moieties.

By “charged moiety” is meant a moiety which loses a proton at physiological pH thereby becoming negatively charged (e.g., carboxylate, or phosphate), a moiety which gains a proton at physiological pH thereby becoming positively charged (e.g., ammonium, guanidinium, or amidinium), a moiety that includes a net formal positive charge without protonation (e.g., quaternary ammonium), or a moiety that includes a net formal negative charge without loss of a proton (e.g., borate, BR₄⁻).

In certain embodiments, charged groups are attached through the ring nitrogen of the phenothiazine.

A charged group may be cationic or an anionic. Charged groups include 3, 4, 5, 6, 7, 8, 9, 10, or more negatively charged moieties and/or 3, 4, 5, 6, 7, 8, 9, 10, or more positively charged moieties. Charged moieties include, without limitation, carboxylate, phosphodiester, phosphoramidate, borate, phosphate, phosphonate, phosphonate ester, sulfonate, sulfate, thiolate, phenolate, ammonium, amidinium, guanidinium, quaternary ammonium, and imidazolium moieties.

In certain embodiments, a charged group has a molecular weight less than 600, 400, 200, or 100 daltons.

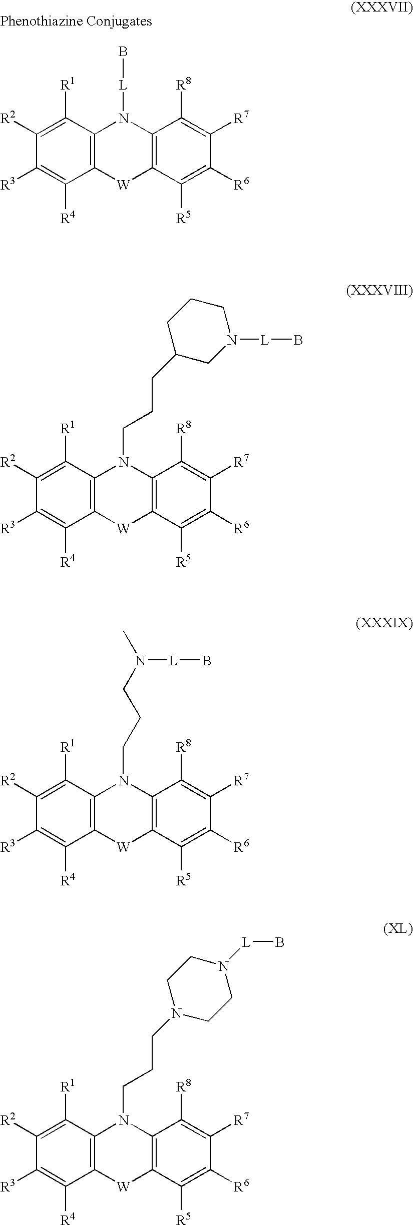

In formulas (XXXVII)-(XL), R¹, R², R³, R⁴, R⁵, R⁶, R⁷, R⁸, and W are as described above. L is a linker of formula (XXXV), described above. B is a bulky or charged group, as described above.

Methods for Preparing Exemplary Phenothiazine Conjugates

1. Protection and Deprotection of Reactive Groups

The synthesis of phenothiazine conjugates may involve the selective protection and deprotection of alcohols, amines, ketones, sulfhydryls or carboxyl functional groups of the phenothiazine, the linker, the bulky group, and/or the charged group. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994.

In the examples that follow, the use of protecting groups is indicated in a structure by the letter P, where P for any amine, aldehyde, ketone, carboxyl, sulfhydryl, or alcohol may be any of the protecting groups listed above.

2. Polyguanidine Conjugates of Phenothiazines

2-(trifluoromethyl)phenothiazine (CAS 92-30-8, Aldrich Cat. No. T6,345-2) can be reacted with an activated carboxyl. Carboxyls can be activated, for example, by formation of an active ester, such as nitrophenylesters, N-hydroxysuccinimidyl esters, or others as described in Chem. Soc. Rev. 12:129, 1983 and Angew. Chem. Int. Ed. Engl. 17:569, 1978, incorporated herein by reference. For example, oxalic acid (Aldrich, catalogue number 24,117-2) can be attached as a linking group, as shown below in reaction 1.

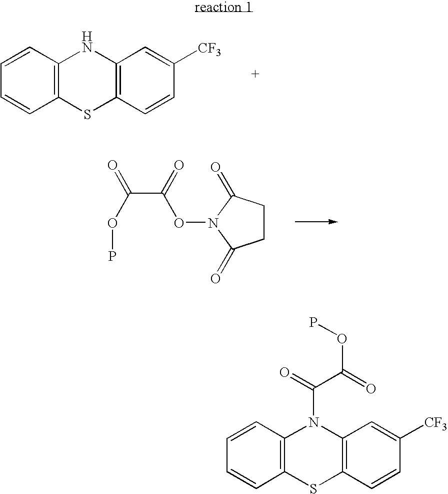

The protecting group in the reaction product can be removed by hydrolysis. The resulting acid is available for conjugation to a bulky group or a charged group.

The polyguanidine peptoid N-hxg, shown below, can be prepared according to the methods described by Wender et al., Proc. Natl. Acad. Sci. USA 97(24):13003-8, 2000, incorporated herein by reference.

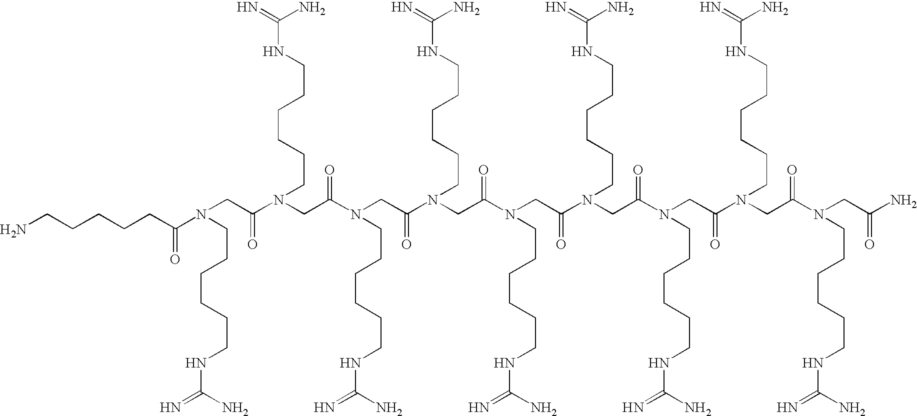

### N-hxg with an Aminohexanoic Acid Linker at the N-Terminus

The carboxyl derivative produced by the deprotection of the product of reaction 1 can be activated, vide supra, and conjugated to the protected precursor of N-hxg followed by the formation of the guanidine moieties and cleavage from the solid phase resin, as described by Wender ibid., to produce the polyguanidine prednisolone conjugate shown below.

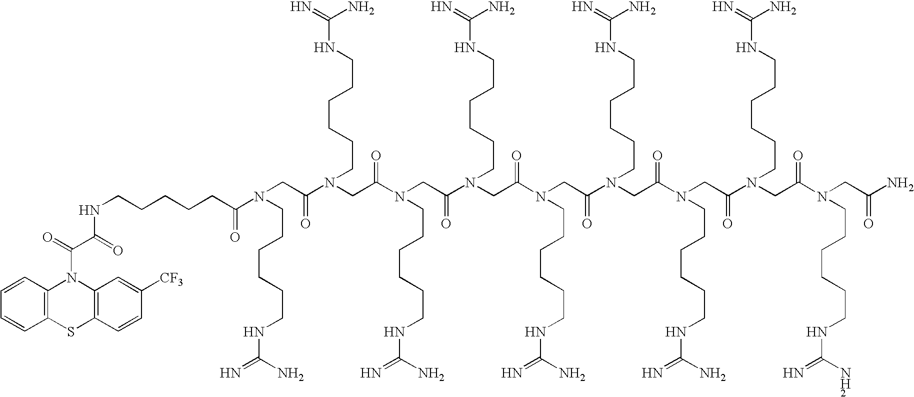

The resulting phenothiazine conjugate includes a bulky group (FW 1,900 Da) which includes several positively charged moieties.

3. Hyaluronic Acid Conjugates of a Phenothiazines

2-Methylthiophenothiazine (CAS 7643-08-5, Aldrich Cat. No. 55, 292-5) can be reacted a hydrazine-substituted carboxylic acid, which has been activated as shown in reaction 3.

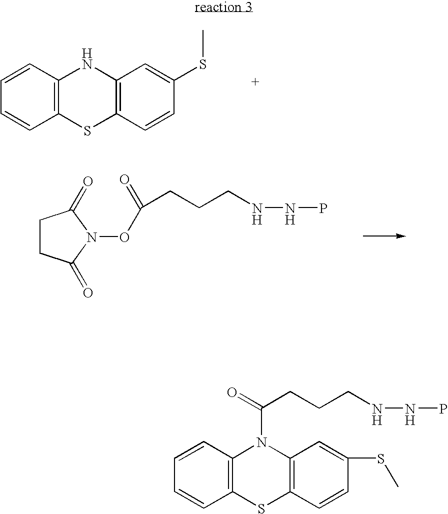

The protecting group can be removed from the reaction product and the free hydrazine coupled to a carboxyl group of hyaluronic acid as described by, for example, Vercruysse et al., Bioconjugate Chem., 8:686, 1997 or Pouyani et al., J. Am. Chem. Soc., 116:7515, 1994. The structure of the resulting hydrazide conjugate is provided below.

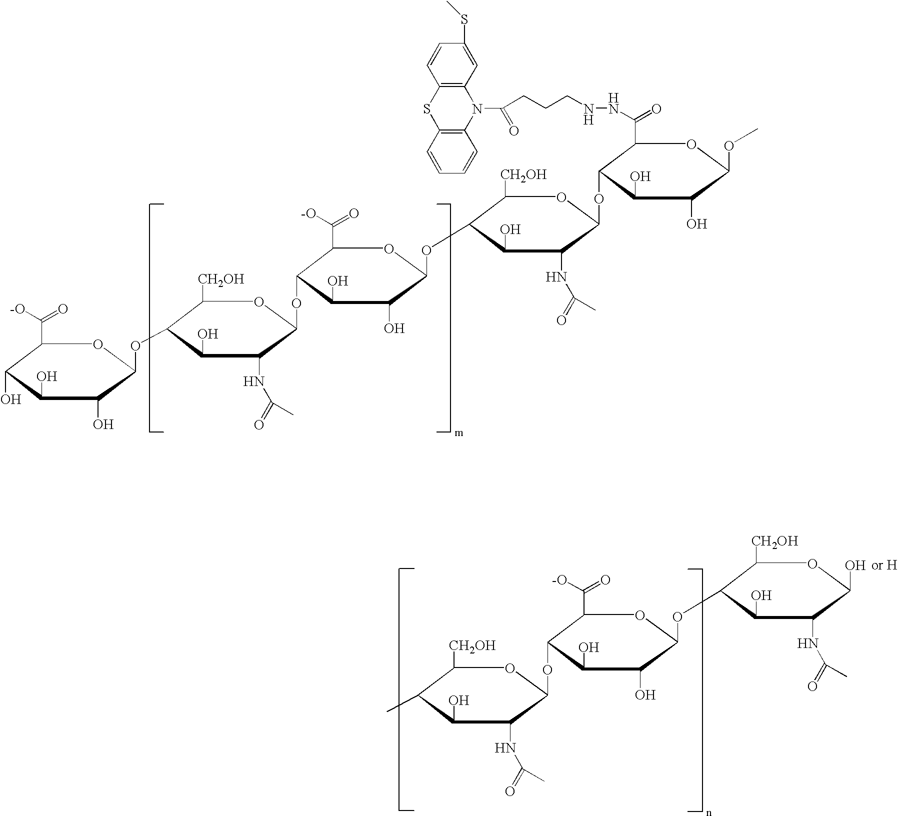

In the phenothiazine conjugate above, the hyaluronic acid is approximately 160,000 Daltons in molecular weight. Accordingly, m and n are whole integers between 0 and 400. Conjugates of lower and higher molecular weight hyaluronic acid can be prepared in a similar fashion.

4. PEG Conjugates of Phenothiazines

(10-piperadinylpropyl)phenothiazine can be conjugated to mono-methyl polyethylene glycol 5,000 propionic acid N-succinimidyl ester (Fluka, product number 85969). The resulting mPEG conjugate, shown below, is an example of a phenothiazine conjugate of a bulky uncharged group. 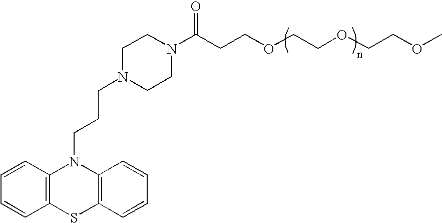
mPEG-phenothiazine, n is approximately 110

Conjugates of lower and higher molecular weight mPEG can be prepared in a similar fashion (see, for example, Roberts et al., Adv. Drug Delivery Rev. 54:459 (2002)).

Chlorpromazine can be conjugated to an activated PEG (e.g., a mesylate, or halogenated PEG compound) as shown in reaction 4.

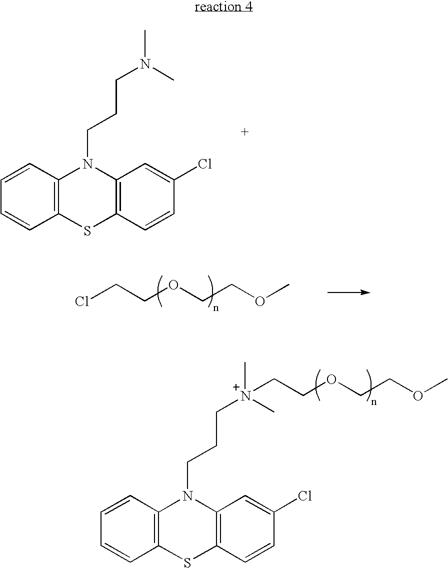

5. Pentamidine Conjugates of Phenothiazines

Pentamadine conjugates of phenothiazine can be prepared using a variety of conjugation techniques. For example, reaction 5 shows perimethazine, the alcohol activated in situ (e.g., using mesylchloride), followed by alkylation of pentamidine to form the conjugate product of the two therapeutic agents. 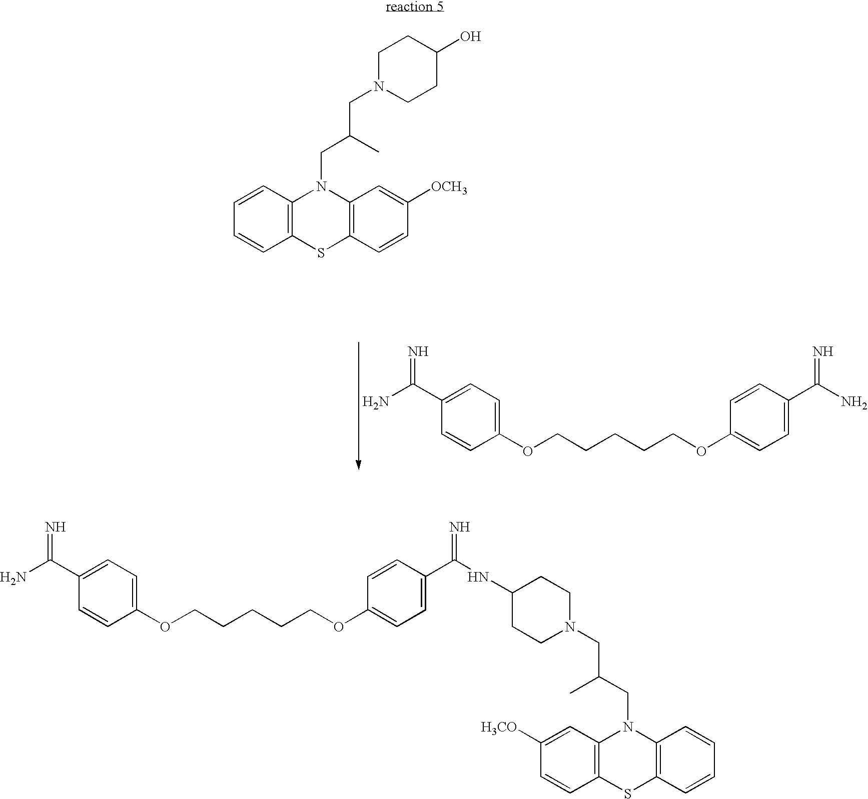
Combinations Comprising Phenothiazines and Antiproliferative Agents

In another aspect, the drug combinations may comprise (a) a compound of formula (XLI):

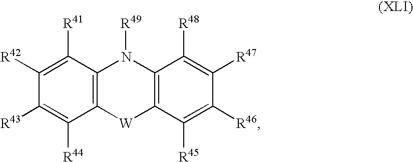

or a pharmaceutically active or acceptable salt thereof, wherein R⁴² is selected from the group consisting of: CF₃, halogen, OCH₃, COCH₃, CN, OCF₃, COCH₂CH₃, CO(CH₂)₂CH₃, S(O)₂CH₃, S(O)₂N(CH₃)₂, and SCH₂CH₃;

R⁴⁹ is selected from the group consisting of:

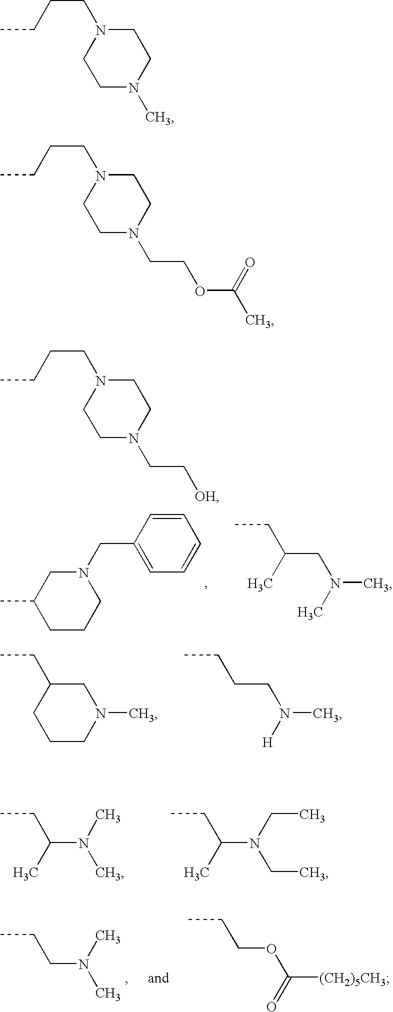

each of R⁴¹, R⁴³, R⁴⁴, R⁴⁵R⁴⁶, R⁴⁷, and R⁴⁸ is independently H, OH, F, OCF₃, or OCH₃; and W is selected from the group consisting of: NO,

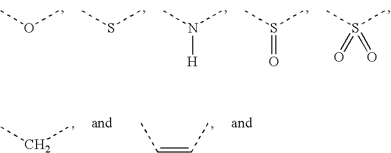

(b) an antiproliferative agent, wherein each are present in amounts that together are sufficient to inhibit the growth of a neoplasm.

Preferably, the compound of formula (XLI) is acepromazine, chlorpromazine, cyamemazine, fluphenazine, mepazine, methotrimeprazine, methoxypromazine, perazine, perphenazine, prochlorperazine, promethazine, propiomazine, thiethylperazine, thiopropazate, thioridazine, trifluoperazine, or triflupromazine.

Antiproliferative agents are described above, such as those in Tables 1 and 2.

In certain embodiments, the drug combination contains an anti-proliferative agent of formula (XLII):

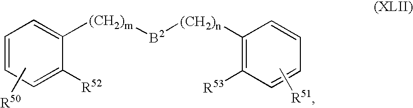

or a pharmaceutically active or acceptable salt thereof. In formula (XLII), B² is

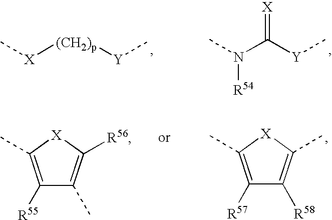

wherein each of X and Y is, independently, O, NR⁵⁹, or S; each of R⁵⁴ and R⁵⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R⁵⁵, R⁵⁶, R⁵⁷, and R⁵⁸ is, independently, H, halogen, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; p is an integer between 2 and 6, inclusive; each of m and n is, independently, an integer between 0 and 2, inclusive; each of R⁵⁰ and R⁵¹ is

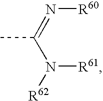

wherein R⁶¹ is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; R⁶² is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, alkoxy, aryloxy, or C₁₋₇ heteroalkyl; and R⁶⁰ is H, OH, or acyl, or R⁶⁰ and R⁶¹ together represent

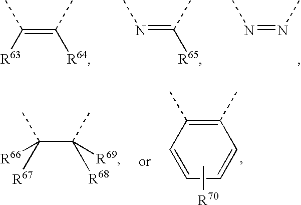

wherein each of R⁶³, R⁶⁴, and R⁶⁵ is independently, H, halogen, trifluoromethyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R⁶⁶, R⁶⁷, R⁶⁸, and R⁶⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁰ is H, halogen, trifluoromethyl, OCF₃, NO₂, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R⁵² and R⁵³ is, independently, H, Cl, Br, OH, OCH₃, OCF₃, NO₂, and NH₂, or R⁵² and R⁵³ together form a single bond.

Compounds of formula (XLII) useful in the methods and compositions of the invention include pentamidine, propamidine, butamidine, heptamidine, nonamidine, stilbamidine, hydroxystilbamidine, diminazene, dibrompropamidine, 2,5-bis(4-amidinophenyl)furan, 2,5-bis(4-amidinophenyl)furan-bis-O-methylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, and 2,4-bis(4-amidinophenyl)thiophene-bis-O-methylamidoxime.

In one embodiment, the compound of formula (XLI) is chlorpromazine, perphenazine or promethazine and the compound of formula (XLII) is pentamidine, 2,5-bis(4-amidinophenyl)furan, or 2,5-bis(4-amidinophenyl)furan-bis-O-methylamidoxime.

The invention also features a drug combination that includes (a) a first compound selected from prochlorperazine, perphenazine, mepazine, methotrimeprazine, acepromazine, thiopropazate, perazine, propiomazine, putaperazine, thiethylperazine, methopromazine, chlorfenethazine, cyamemazine, perphenazine, norchlorpromazine, trifluoperazine, thioridazine (or a salt of any of the above), and dopamine D2 antagonists (e.g., sulpride, pimozide, spiperone, ethopropazine, clebopride, bupropion, and haloperidol), and, (b) a second compound selected from pentamidine, propamidine, butamidine, heptamidine, nonamidine, stilbamidine, hydroxystilbamidine, diminazene, benzamidine, phenamidine, dibrompropamidine, 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino)phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis{4-(2-imidazolinyl)}phenyl]-3-(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorine, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan, or a salt of any of the above.

Alternatively, the second compound can be a functional analog of pentamidine, such as netropsin, distamycin, bleomycin, actinomycin, daunorubicin, or a compound that falls within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, or U.S. Patent Application Publication Nos. US 2001/0044468 A1 and US 2002/0019437 A1.

Combinations Comprising Kinesin Inhibitors and Antiproliferative Agents

In certain embodiments, the drug combinations of the present invention may comprise kinesin inhibitors and antiproliferative agents (e.g., Group A and Group B antiproliferative agents).

Kinesin Inhibitors

By “kinesin inhibitor” is meant a compound that inhibits by a statistically significant amount (e.g., by at least 10%, 20%, 30%, or more) the enzymatic activity of a mitotic kinesin (e.g., HsEg5). Mitotic kinesins are enzymes essential for assembly and function of the mitotic spindle and play essential roles during all phases of mitosis. Perturbation of mitotic kinesin function causes malformation or dysfunction of the mitotic spindle, frequently resulting in cell cycle arrest and cell death. Kinesin inhibitors can be identified using a variety of methods as disclosed in PCT publication WO02/057244. For example, kinesin inhibition can be identified using assays for cell cycle distribution, cell viability, morphology, activity, or by monitoring the formation of mitotic spindles.

Methods for monitoring cell cycle distribution of a cell population include, for example, flow cytometry. Kinesin inhibitors include, without limitation, chlorpromazine, monasterol, terpendole E, HR22C16, and SB715992. Other mitotic kinesin inhibitors are those compounds disclosed in Hopkins et al., Biochemistry 39:2805, 2000, Hotha et al., Angew Chem. Inst. Ed. 42:2379, 2003, PCT Publication Nos. WO01/98278, WO02/057244, WO02/079169, WO02/057244, WO02/056880, WO03/050122, WO03/050064, WO03/049679, WO03/049678, WO03/049527, WO03/079973, and WO03/039460; U.S. Patent Application Publication Nos. 2002/0165240, 2003/0008888, 2003/0127621, and 2002/0143026; and U.S. Pat. Nos. 6,437,115, 6,545,004, 6,562,831, 6,569,853, and 6,630,479.

In certain embodiments, the kinesin inhibitors are phenothiazines, analogs or metabolites. Such compounds are described above in the sections related to combinations comprising chlorpromazine and pentamidine and to combinations comprising phenothiazine conjugates or phenothiazines and antiproliferative agents.

In certain embodiments, the kinesin inhibitor may be a compound having the formula (XLIII): 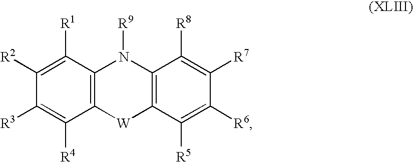
or a pharmaceutically acceptable salt thereof,

wherein R² is CF₃, halogen, OCH₃, COCH₃, CN, OCF₃, COCH₂CH₃, CO(CH₂)₂CH₃, or SCH₂CH₃;

R⁹ is selected from:

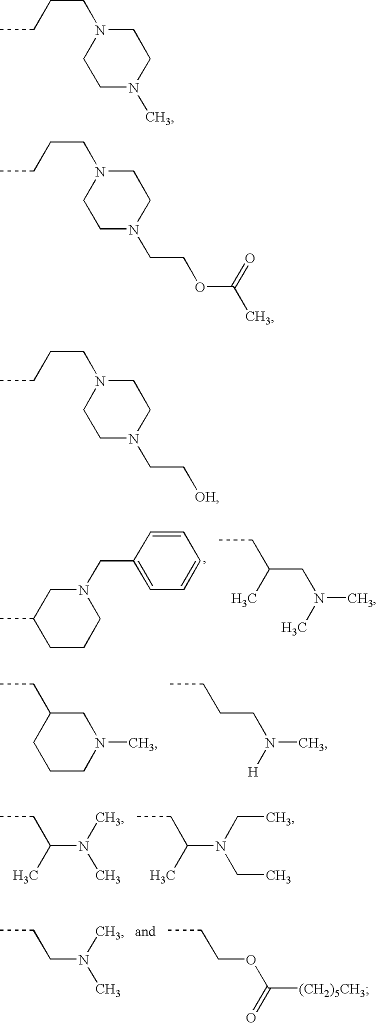

or R⁹ has the formula: 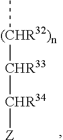
wherein n is 0 or 1, Z is NR³⁵R³⁶ or OR³⁷; each of R³², R³³, R³⁴, R³⁵, R³⁶, and R³⁷ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; or any of R³³, R³⁴, R³⁵, R³⁶, and R³⁷ can be optionally taken together with intervening carbon or non-vicinal O, S, or N atoms to form one or more five- to seven-membered rings, optionally substituted by H, halogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl;

each of R¹, R³, R⁴, R⁵, R⁶, R⁷, and R⁸ is independently H, OH, F, OCF₃, or OCH₃; and

W is NO,

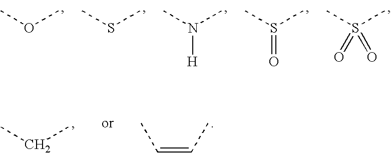

Exemplary kinesin inhibitors include acepromazine, chlorfenethazine, chlorpromazine, N-methyl chlorpromazine, cyamemazine, fluphenazine, mepazine, methotrimeprazine, methoxypromazine, norchlorpromazine, perazine, perphenazine, phenothiazine, prochlorperazine, promethazine, propiomazine, putaperazine, thiethylperazine, thiopropazate, thioridazine, trifluoperazine, or triflupromazine.

Antiproliferative Agents

Antiproliferative agents are described above. In certain embodiments, antiproliferative agents are Group A antiproliferative agents (e.g., those listed in Table 4). In certain embodiments, the antiproliferative agents are not pentamidines or their analogs, endo-exonuclease inhibitors, PRL phosphatase inhibitors, or PTP1B inhibitors.

In certain embodiments, Group A antiproliferative agents may be an alkylating agent (e.g., dacarbazine), an anthracycline (e.g., mitoxantrone), an anti-estrogen (e.g., bicalutamide), an anti-metabolite (e.g., floxuridine), a microtubule binding, stabilizing agent (e.g., docetaxel), microtubule binding, destabilizing agent (e.g., vinorelbine), topoisomerase inhibitor (e.g., hydroxycamptothecin (SN-38)), or a kinase inhibitor (e.g., a tyrphostin, such as AG1478). In certain embodiments, the agent is altretamine, carmustine, chlorambucil, cyclophosphamide, dacarbazine, ifosfamide, melphalan, mitomycin, temozolomide, doxorubicin, epirubicin, mitoxantrone, anastrazole, bicalutamide, estramustine, exemestane, flutamide, fulvestrant, tamoxifen, toremifene, capecitabine, floxuridine, fluorouracil, gemcitabine, hydroxyurea, methotrexate, gleevec, tyrphostin, docetaxel, pacilitaxel, vinblastine, vinorelbine, adjuvant/enhancing agents (celecoxib, gallium, isotretinoin, leucovorin, levamisole, pamidronate, suramin), or agents such as thalidomide, carboplatin, cisplatin, oxaliplatin, etoposide, hydroxycamptothecin, irinotecan, or topotecan. In certain other embodiments, the Group A antiproliferative agent is selected from carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, or rituximab, or any combination thereof.

In certain embodiments, the antiproliferative agent may be a bis-benzimidazole compound.

By “bis-benzimidazole compound” is meant a compound of formula (XLIV):

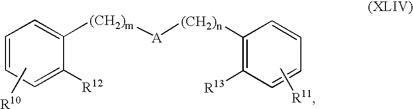

wherein A is selected from:

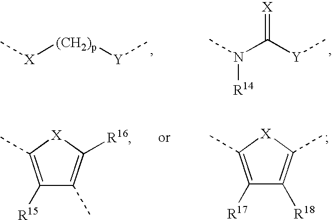

each of X and Y is, independently, O, NR¹⁹, or S; each of R¹⁴ and R¹⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R¹⁵, R¹⁶, R¹⁷, and R¹⁸ is, independently, H, halogen, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; p is an integer between 2 and 6, inclusive; each of m and n is, independently, an integer between 0 and 2, inclusive; each of R¹⁰ and R¹¹ is

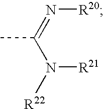

each of R²¹ and R²² is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, acyl, or C₁₋₇ heteroalkyl; R²⁰ is H, OH, or acyl, or R²⁰ and R²¹ together represent

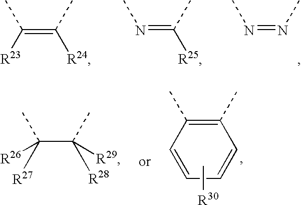

each of R²³, R²⁴, and R²⁵ is, independently, H, halogen, trifluoromethyl, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R²⁶, R²⁷, R²⁸, and R²⁹ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁰ is H, halogen, trifluoromethyl, OCF₃, NO₂, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, alkoxy, arlyoxy, or C₁₋₇ heteroalkyl; each of R¹² and R¹³ is, independently, H, Cl, Br, OH, OCH₃, OCF₃, NO₂, and NH₂, or R¹² and R¹³ together form a single bond. Bis-benzimidazole compounds include pentamidine, propamidine, butamidine, heptamidine, nonamidine, stilbamidine, hydroxystilbamidine, diminazene, berenil, benzamidine, phenamidine, dibrompropamidine, 1,3-bis(4-amidino-2-methoxyphenoxy)propane, phenamidine, amicarbalide, 1,5-bis(4′-(N-hydroxyamidino)phenoxy)pentane, 1,3-bis(4′-(N-hydroxyamidino)phenoxy)propane, 1,3-bis(2′-methoxy-4′-(N-hydroxyamidino) phenoxy)propane, 1,4-bis(4′-(N-hydroxyamidino)phenoxy)butane, 1,5-bis(4′-(N-hydroxyamidino)phenoxy)pentane, 1,4-bis(4′-(N-hydroxyamidino)phenoxy)butane, 1,3-bis(4′-(4-hydroxyamidino)phenoxy)propane, 1,3-bis(2′-methoxy-4′-(N-hydroxyamidino)phenoxy)propane, 2,5-bis[4-amidinophenyl]furan, 2,5-bis[4-amidinophenyl]furan-bis-amidoxime, 2,5-bis[4-amidinophenyl]furan-bis-O-methylamidoxime, 2,5-bis[4-amidinophenyl]furan-bis-O-ethylamidoxime, 2,5-bis(4-amidinophenyl)furan-bis-O-4-fluorophenyl, 2,5-bis(4-amidinophenyl)furan-bis-O-4-methoxyphenyl, 2,4-bis(4-amidinophenyl)furan, 2,4-bis(4-amidinophenyl)furan-bis-O-methylamidoxime, 2,4-bis(4-amidinophenyl)furan-bis-O-4-fluorophenyl, 2,4-bis(4-amidinophenyl)furan-bis-O-4-methoxyphenyl, 2,5-bis(4-amidinophenyl) thiophene, 2,5-bis(4-amidinophenyl) thiophene-bis-O-methylamidoxime, 2,4-bis(4-amidinophenyl)thiophene, 2,4-bis(4-amidinophenyl)thiophene-bis-O-methylamidoxime, 2,8-diamidinodibenzothiophene, 2,8-bis(N-isopropylamidino)carbazole, 2,8-bis(N-hydroxyamidino)carbazole, 2,8-bis(2-imidazolinyl)dibenzothiophene, 2,8-bis(2-imidazolinyl)-5,5-dioxodibenzothiophene, 3,7-diamidinodibenzothiophene, 3,7-bis(N-isopropylamidino)dibenzothiophene, 3,7-bis(N-hydroxyamidino)dibenzothiophene, 3,7-diaminodibenzothiophene, 3,7-dibromodibenzothiophene, 3,7-dicyanodibenzothiophene, 2,8-diamidinodibenzofuran, 2,8-di(2-imidazolinyl)dibenzofuran, 2,8-di(N-isopropylamidino)dibenzofuran, 2,8-di(N-hydroxylamidino)dibenzofuran, 3,7-di(2-imidazolinyl)dibenzofuran, 3,7-di(isopropylamidino)dibenzofuran, 3,7-di(N-hydroxylamidino)dibenzofuran, 2,8-dicyanodibenzofuran, 4,4′-dibromo-2,2′-dinitrobiphenyl, 2-methoxy-2′-nitro-4,4′-dibromobiphenyl, 2-methoxy-2′-amino-4,4′-dibromobiphenyl, 3,7-dibromodibenzofuran, 3,7-dicyanodibenzofuran, 2,5-bis(5-amidino-2-benzimidazolyl)pyrrole, 2,5-bis[5-(2-imidazolinyl)-2-benzimidazolyl]pyrrole, 2,6-bis[5-(2-imidazolinyl)-2-benzimidazolyl]pyridine, 1-methyl-2,5-bis(5-amidino-2-benzimidazolyl)pyrrole, 1-methyl-2,5-bis[5-(2-imidazolyl)-2-benzimidazolyl]pyrrole, 1-methyl-2,5-bis[5-(1,4,5,6-tetrahydro-2-pyrimidinyl)-2-benzimidazolyl]pyrrole, 2,6-bis(5-amidino-2-benzimidazoyl)pyridine, 2,6-bis[5-(1,4,5,6-tetrahydro-2-pyrimidinyl)-2-benzimidazolyl]pyridine, 2,5-bis(5-amidino-2-benzimidazolyl)furan, 2,5-bis-[5-(2-imidazolinyl)-2-benzimidazolyl]furan, 2,5-bis-(5-N-isopropylamidino-2-benzimidazolyl)furan, 2,5-bis-(4-guanylphenyl)furan, 2,5-bis(4-guanylphenyl)-3,4-dimethylfuran, 2,5-bis{p-[2-(3,4,5,6-tetrahydropyrimidyl)phenyl]}furan, 2,5-bis[4-(2-imidazolinyl)phenyl]furan, 2,5 [bis-{4-(2-tetrahydropyrimidinyl)}phenyl]-3-(p-tolyloxy)furan, 2,5 [bis{4-(2-imidazolinyl)}phenyl]-3-[(p-tolyloxy)furan, 2,5-bis{4-[5-(N-2-aminoethylamido)benzimidazol-2-yl]phenyl}furan, 2,5-bis[4-(3a,4,5,6,7,7a-hexahydro-1H-benzimidazol-2-yl)phenyl]furan, 2,5-bis[4-(4,5,6,7-tetrahydro-1H-1,3-diazepin-2-yl)phenyl]furan, 2,5-bis(4-N,N-dimethylcarboxhydrazidephenyl)furan, 2,5-bis{4-[2-(N-2-hydroxyethyl)imidazolinyl]phenyl}furan, 2,5-bis[4-(N-isopropylamidino)phenyl]furan, 2,5-bis{4-[3-(dimethylaminopropyl)amidino]phenyl}furan, 2,5-bis{4-[N-(3-aminopropyl)amidino]phenyl}furan, 2,5-bis[2-(imidzaolinyl)phenyl]-3,4-bis(methoxymethyl)furan, 2,5-bis[4-N-(dimethylaminoethyl)guanyl]phenylfuran, 2,5-bis{4-[(N-2-hydroxyethyl)guanyl]phenyl}furan, 2,5-bis[4-N-(cyclopropylguanyl)phenyl]furan, 2,5-bis[4-(N,N-diethylaminopropyl)guanyl]phenylfuran, 2,5-bis{4-[2-(N-ethylimidazolinyl)]phenyl}furan, 2,5-bis{4-[N-(3-pentylguanyl)]}phenylfuran, 2,5-bis[4-(2-imidazolinyl)phenyl]-3-methoxyfuran, 2,5-bis[4-(N-isopropylamidino)phenyl]-3-methylfuran, bis[5-amidino-2-benzimidazolyl]methane, bis[5-(2-imidazolyl)-2-benzimidazolyl]methane, 1,2-bis[5-amidino-2-benzimidazolyl]ethane, 1,2-bis[5-(2-imidazolyl)-2-benzimidazolyl]ethane, 1,3-bis[5-amidino-2-benzimidazolyl]propane, 1,3-bis[5-(2-imidazolyl)-2-benzimidazolyl]propane, 1,4-bis[5-amidino-2-benzimidazolyl]propane, 1,4-bis[5-(2-imidazolyl)-2-benzimidazolyl]butane, 1,8-bis[5-amidino-2-benzimidazolyl]octane, trans-1,2-bis[5-amidino-2-benzimidazolyl]ethene, 1,4-bis[5-(2-imidazolyl)-2-benzimidazolyl]-1-butene, 1,4-bis[5-(2-imidazolyl)-2-benzimidazolyl]-2-butene, 1,4-bis[5-(2-imidazolyl)-2-benzimidazolyl]-1-methylbutane, 1,4-bis[5-(2-imidazolyl)-2-benzimidazolyl]-2-ethylbutane, 1,4-bis[5-(2-imidazolyl)-2-benzimidazolyl]-1-methyl-1-butene, 1,4-bis[5-(2-imidazolyl)-2-benzimidazolyl]-2,3-diethyl-2-butene, 1,4-bis[5-(2-imidazolyl)-2-benzimidazolyl]-1,3-butadiene, 1,4-bis[5-(2-imidazolyl)-2-benzimidazolyl]-2-methyl-1,3-butadiene, bis[5-(2-pyrimidyl)-2-benzimidazolyl]methane, 1,2-bis[5-(2-pyrimidyl)-2-benzimidazolyl]ethane, 1,3-bis[5-amidino-2-benzimidazolyl]propane, 1,3-bis[5-(2-pyrimidyl)-2-benzimidazolyl]propane, 1,4-bis[5-(2-pyrimidyl)-2-benzimidazolyl]butane, 1,4-bis[5-(2-pyrimidyl)-2-benzimidazolyl]-1-butene, 1,4-bis[5-(2-pyrimidyl)-2-benzimidazolyl]-2-butene, 1,4-bis[5-(2-pyrimidyl)-2-benzimidazolyl]-1-methylbutane, 1,4-bis[5-(2-pyrimidyl)-2-benzimidazolyl]-2-ethylbutane, 1,4-bis[5-(2-pyrimidyl)-2-benzimidazolyl]-1-methyl-1-butene, 1,4-bis[5-(2-pyrimidyl)-2-benzimidazolyl]-2,3-diethyl-2-butene, 1,4-bis[5-(2-pyrimidyl)-2-benzimidazolyl]-1,3-butadiene, and 1,4-bis[5-(2-pyrimidyl)-2-benzimidazolyl]-2-methyl-1,3-butadiene, 2,4-bis(4-guanylphenyl)pyrimidine, 2,4-bis(4-imidazolin-2-yl)pyrimidine, 2,4-bis[(tetrahydropyrimidinyl-2-yl)phenyl]pyrimidine, 2-(4-[N-i-propylguanyl]phenyl)-4-(2-methoxy-4-[N-i-propylguanyl]phenyl)pyrimidine, 4-(N-cyclopentylamidino)-1,2-phenylene diamine, 2,5-bis-[2-(5-amidino)benzimidazoyl]furan, 2,5-bis[2-{5-(2-imidazolino)}benzimidazoyl]furan, 2,5-bis[2-(5-N-isopropylamidino)benzimidazoyl]furan, 2,5-bis[2-(5-N-cyclopentylamidino)benzimidazoyl]furan, 2,5-bis[2-(5-amidino)benzimidazoyl]pyrrole, 2,5-bis[2-{5-(2-imidazolino)}benzimidazoyl]pyrrole, 2,5-bis[2-(5-N-isopropylamidino)benzimidazoyl]pyrrole, 2,5-bis[2-(5-N-cyclopentylamidino)benzimidazoyl]pyrrole, 1-methyl-2,5-bis[2-(5-amidino)benzimidazoyl]pyrrole, 2,5-bis[2-{5-(2-imidazolino)}benzimidazoyl]-1-methylpyrrole, 2,5-bis[2-(5-N-cyclopentylamidino)benzimidazoyl]-1-methylpyrrole, 2,5-bis[2-(5-N-isopropylamidino)benzimidazoyl]thiophene, 2,6-bis[2-{5-(2-imidazolino)}benzimidazoyl]pyridine, 2,6-bis[2-(5-amidino)benzimidazoyl]pyridine, 4,4′-bis[2-(5-N-isopropylamidino)benzimidazoyl]-1,2-diphenylethane, 4,4′-bis[2-(5-N-cyclopentylamidino)benzimidazoyl]-2,5-diphenylfuran, 2,5-bis[2-(5-amidino)benzimidazoyl]benzo[b]furan, 2,5-bis[2-(5-N-cyclopentylamidino)benzimidazoyl]benzo[b]furan, 2,7-bis[2-(5-N-isopropylamidino)benzimidazoyl]fluorine, 2,5-bis[4-(3-(N-morpholinopropyl)carbamoyl)phenyl]furan, 2,5-bis[4-(2-N,N-dimethylaminoethylcarbamoyl)phenyl]furan, 2,5-bis[4-(3-N,N-dimethylaminopropylcarbamoyl)phenyl]furan, 2,5-bis[4-(3-N-methyl-3-N-phenylaminopropylcarbamoyl)phenyl]furan, 2,5-bis[4-(3-N,N⁸,N¹¹-trimethylaminopropylcarbamoyl)phenyl]furan, 2,5-bis[3-amidinophenyl]furan, 2,5-bis[3-(N-isopropylamidino)amidinophenyl]furan, 2,5-bis[3[(N-(2-dimethylaminoethyl)amidino]phenylfuran, 2,5-bis[4-(N-2,2,2-trichloroethoxycarbonyl)amidinophenyl]furan, 2,5-bis[4-(N-thioethylcarbonyl) amidinophenyl]furan, 2,5-bis[4-(N-benzyloxycarbonyl)amidinophenyl]furan, 2,5-bis[4-(N-phenoxycarbonyl)amidinophenyl]furan, 2,5-bis[4-(N-(4-fluoro)-phenoxycarbonyl)amidinophenyl]furan, 2,5-bis[4-(N-(4-methoxy)phenoxycarbonyl)amidinophenyl]furan, 2,5-bis[4(1-acetoxyethoxycarbonyl)amidinophenyl]furan, and 2,5-bis[4-(N-(3-fluoro)phenoxycarbonyl)amidinophenyl]furan, or a salt of any of the above. Bis-benzimidazole compounds also include functional analogs of pentamidine, such as netropsin, distamycin, bleomycin, actinomycin, daunorubicin. Bis-benzimidazole compounds further include any compound that falls within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, and any compound that falls within a formula provided in any of U.S. Patent Application Publication Nos. US 2001/0044468 A1 and US 2002/0019437 A1. Bis-benzimidazole compounds include any compound identified as a pentamidine analog, or falling within a formula that includes pentamidine, provided in U.S. Pat. No. 6,569,853 and in U.S. Patent Application Publication No. 20040116407 A1.

Exemplary Drug Combinations

In certain embodiments, the drug combinations of the present invention comprise (1) a kinesin inhibitor selected from acepromazine, chlorfenethazine, chlorpromazine, N-methyl chlorpromazine, cyamemazine, fluphenazine, mepazine, methotrimeprazine, methoxypromazine, norchlorpromazine, perazine, perphenazine, phenothiazine, prochlorperazine, promethazine, propiomazine, putaperazine, thiethylperazine, thiopropazate, thioridazine, trifluoperazine, or triflupromazine, and (2) a Group A antiproliferative agent selected from dacarbazine, mitoxantrone, bicalutamide, floxuridine, leucovorin, vinblastine, vinorelbine, hydroxycamptothecin, tyrphostin, docetaxel, or combinations thereof.

In certain other embodiments, the drug combinations of the present invention comprises (1) a kinesin inhibitor selected from acepromazine, chlorfenethazine, chlorpromazine, N-methyl chlorpromazine, cyamemazine, fluphenazine, mepazine, methotrimeprazine, methoxypromazine, norchlorpromazine, perazine, perphenazine, phenothiazine, prochlorperazine, promethazine, propiomazine, putaperazine, thiethylperazine, thiopropazate, thioridazine, trifluoperazine, or triflupromazine, and (2) a Group A antiproliferative agent selected from carmustine, cisplatin, etoposide, melphalan, mercaptopurine, methotrexate, mitomycin, vinblastine, paclitaxel, docetaxel, vincristine, vinorelbine, cyclophosphamide, chlorambucil, gemcitabine, capecitabine, 5-fluorouracil, fludarabine, raltitrexed, irinotecan, topotecan, doxorubicin, epirubicin, letrozole, anastrazole, formestane, exemestane, tamoxifen, toremofine, goserelin, leuporelin, bicalutamide, flutamide, nilutamide, hypericin, trastuzumab, rituximab, or combinations thereof.

In certain embodiments, when the drug combinations comprise trifluoperazine, the antiproliferative agents in the combinations are not doxorubicin, aclacinomycin, trifluoroacetyladriamycin-14-valerate, vinblastine, dactinomycin, colchicine, or adriamycin.

In certain other embodiments, when the drug combinations comprise chlorpromazine, the antiproliferative agents in the combinations are not paclitaxel, doxorubicin, vinblastine, dactinomycin, or colchicines.

In certain other embodiments, when the drug combinations comprise thioridazine, the antiproliferative agents in the combinations are not doxorubicin, vinblastine, dactinomycin, or colchicine.

In certain embodiments, the drug combinations of the present invention comprise chlorpromazine and dacarbazine, chlorpromazine and floxuridine, chlorpromazine and tyrphostin 1486, chlorpromazine and vinblastine, chlorpromazine and hydroxycamptothecin, chlorpromazine and leucovorin, chlorpromazine and paclitaxel, or chlorpromazine and docetaxel.

Combinations Comprising Mitotic Kinesin Inhibitors and Protein Tyrosine Phosphatase Inhibitors

In certain embodiments, the drug combinations of the present invention comprise agents that reduce the biological activity of a mitotic kinesin and agents that reduce the biological activity of protein tyrosine phosphatases. In certain embodiments, the drug combinations further comprise one or more antiproliferative agents.

Mitotic Kinesins

Mitotic kinesins are essential motors in mitosis. They control spindle assembly and maintenance, attachment and proper positioning of the chromosomes to the spindle, establish the bipolar spindle and maintain forces in the spindle to allow movement of chromosomes toward opposite poles. Perturbations of mitotic kinesin function cause malformation or dysfunction of the mitotic spindle, frequently resulting in cell cycle arrest and cell death.

Exemplary mitotic kinesins include HsEg5/KSP, KIFC3, CHO2, MKLP, MCAK, Kin2, Kif4, MPP1, CENP-E, NYREN62, LOC8464, and KIF8. Other mitotic kinesins are described in U.S. Pat. Nos. 6,414,121, 6,582,958, 6,544,766, 6,492,158, 6,455,293, 6,440,731, 6,437,115, 6,420,162, 6,399,346, 6,395,540, 6,383,796, 6,379,941, and 6,248,594. The GenBank Accession Nos. of representative mitotic kinesins are provided below.

Human mitotic kinesins

| Protein name | GenBank Accession No. |
|---|---|
| Eg5/KSP | AA857025, U37426, X85137 |
| KIFC3 | BC001211 |
| MKLP1 | AI131325, AU133373, X67155 |
| MCAK | AL046197, U63743 |
| KIN2 | Y08319 |
| KIF4 | AF071592 |
| MPP1 | AL117496 |
| CENP-E | Z15005 |
| CHO2 | AL021366 |
| HsNYREN62 | AF155117 |
| HsLOC8464 | NM_032559 |
| KIF8 | AB001436 |

HsEg5/KSP has been cloned and characterized (see, e.g., Blangy et al., Cell, 83:1159-69 (1995); Galgio et al., J. Cell Biol., 135:399-414, 1996; Whitehead et al., J. Cell Sci., 111:2551-2561, 1998; Kaiser, et al., J. Biol. Chem., 274:18925-31, 1999; GenBank accession numbers: X85137, NM 004523). Drosophila (Heck et al., J. Cell Biol., 123:665-79, 1993) and Xenopus (Le Guellec et al., Mol. Cell Biol., 11:3395-8, 1991) homologs of KSP have been reported. Drosophila KLP61F/KRP130 has reportedly been purified in native form (Cole, et al., J. Biol. Chem., 269:22913-22916, 1994), expressed in E. coli, (Barton, et al., Mol. Biol. Cell, 6:1563-74, 1995) and reported to have motility and ATPase activities (Cole, et al., supra; Barton, et al., supra). Xenopus Eg5/KSP was expressed in E. coli and reported to possess motility activity (Sawin, et al., Nature, 359:540-3, 1992; Lockhart and Cross, Biochemistry, 35:2365-73, 1996; Crevel, et al., J. Mol. Biol., 273:160-170, 1997) and ATPase activity (Lockhart and Cross, supra; Crevel et al., supra).

Besides KSP, other members of the BimC family include BimC, CIN8, cut7, KIP1, KLP61F (Barton et al., Mol. Biol. Cell. 6:1563-1574, 1995; Cottingham & Hoyt, J. Cell Biol. 138:1041-1053, 1997; DeZwaan et al., J. Cell Biol. 138:1023-1040, 1997; Gaglio et al., J. Cell Biol. 135:399-414, 1996; Geiser et al., Mol. Biol. Cell 8:1035-1050, 1997; Heck et al., J. Cell Biol. 123:665-679, 1993; Hoyt et al., J. Cell Biol. 118:109-120, 1992; Hoyt et al., Genetics 135:35-44, 1993; Huyett et al., J. Cell Sci. 111:295-301, 1998; Miller et al., Mol. Biol. Cell 9:2051-2068, 1998; Roof et al., J. Cell Biol. 118:95-108, 1992; Sanders et al., J. Cell Biol. 137:417-431, 1997; Sanders et al., Mol. Biol. Cell 8:1025-0133, 1997; Sanders et al., J. Cell Biol. 128:617-624, 1995; Sanders & Hoyt, Cell 70:451-458, 1992; Sharp et al., J. Cell Biol. 144:125-138, 1999; Straight et al., J. Cell Biol. 143:687-694, 1998; Whitehead & Rattner, J. Cell Sci. 111:2551-2561, 1998; Wilson et al., J. Cell Sci. 110:451-464, 1997).

Mitotic kinesin biological activities include its ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes, such as kinases or proteases; and specific kinesin cellular activities such as spindle pole separation.

Methods for assaying biological activity of a mitotic kinesin are well known in the art. For example, methods of performing motility assays are described, e.g., in Hall, et al., 1996, Biophys. J., 71:3467-3476, Turner et al., 1996, Anal. Biochem. 242:20-25; Gittes et al., 1996, Biophys. J. 70:418-429; Shirakawa et al., 1995, J. Exp. Biol. 198: 1809-1815; Winkelmann et al., 1995, Biophys. J. 68: 2444-2453; and Winkelmann et al., 1995, Biophys. J. 68:72 S. Methods known in the art for determining ATPase hydrolysis activity also can be used. U.S. application Ser. No. 09/314,464, filed May 18, 1999, hereby incorporated by reference in its entirety, describes such assays. Other methods can also be used. For example, Pᵢ release from kinesin can be quantified. In one embodiment, the ATP hydrolysis activity assay utilizes 0.3 M perchloric acid (PCA) and malachite green reagent (8.27 mM sodium molybdate II, 0.33 mM malachite green oxalate, and 0.8 mM Triton X-100). To perform the assay, 10 μL of reaction is quenched in 90 μL of cold 0.3 M PCA. Phosphate standards are used so data can be converted to nM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to nM Pᵢ and plotted over time. Additionally, ATPase assays known in the art include the luciferase assay.

ATPase activity of kinesin motor domains also can be used to monitor the effects of modulating agents. In one embodiment ATPase assays of kinesin are performed in the absence of microtubules. In another embodiment, the ATPase assays are performed in the presence of microtubules. Different types of modulating agents can be detected in the above assays. In one embodiment, the effect of a modulating agent is independent of the concentration of microtubules and ATP. In another embodiment, the effect of the agents on kinesin ATPase may be decreased by increasing the concentrations of ATP, microtubules, or both. In yet another embodiment, the effect of the modulating agent is increased by increasing concentrations of ATP, microtubules, or both.

Agents that reduce the biological activity of a mitotic kinesin in vitro may then be screened in vivo. Methods for in vivo screening include assays of cell cycle distribution, cell viability, or the presence, morphology, activity, distribution, or amount of mitotic spindles. Methods for monitoring cell cycle distribution of a cell population, for example, by flow cytometry, are well known to those skilled in the art, as are methods for determining cell viability (see, e.g., U.S. Pat. No. 6,617,115).

Mitotic Kinesin Inhibitors

By “mitotic kinesin inhibitor” is meant an agent that binds a mitotic kinesin and reduces, by a significant amount (e.g., by at least 10%, 20% 30% or more), the biological activity of that mitotic kinesin. Mitotic kinesin biological activities include enzymatic activity (e.g., ATPase activity), motor activity (e.g., generation of force) and binding activity (e.g., binding of the motor to either microtubules or its cargo).

Mitotic kinesin inhibitors include chlorpromazine, monasterol, terpendole E, HR22C16, and SB715992. Other mitotic kinesin inhibitors are those compounds disclosed in Hopkins et al., Biochemistry 39:2805, 2000, Hotha et al., Angew Chem. Inst. Ed. 42:2379, 2003, PCT Publication Nos. WO01/98278, WO02/057244, WO02/079169, WO02/057244, WO02/056880, WO03/050122, WO03/050064, WO03/049679, WO03/049678, WO03/049527, WO03/079973, and WO03/039460, and U.S. Patent Application Publication Nos. 2002/0165240, 2003/0008888, 2003/0127621, and 2002/0143026; and U.S. Pat. Nos. 6,437,115, 6,545,004, 6,562,831, 6,569,853, and 6,630,479, and the chlorpromazine analogs described in U.S. patent application Ser. No. 10/617,424, which are also described above.

Protein Tyrosine Phosphatases

Protein tyrosine phosphatases (PTPases) are intracellular signaling molecules that dephosphorylate a tyrosine residue on a protein substrate, thereby modulating certain cellular functions. In normal cells, they typically act in concert with protein tyrosine kinases to regulate signaling cascades through the phosphorylation of protein tyrosine residues. Phosphorylation and dephosphorylation of the tyrosine residues on proteins controls cell growth and proliferation, cell cycle progression, cytoskeletal integrity, differentiation and metabolism. In various metastatic and cancer cell lines, PTP1B and the family of Phosphatases of Regenerating Liver (PRL-1, PRL-2, and PRL-3) have been shown to be overexpressed. For example, PRL-3 (also known as PTP4A3) is expressed in relatively high levels in metatstatic colorectal cancers (Saha et al., Science 294: 1343-1346, 2001.). PRL-1 localizes to the mitotic spindle and is required for mitotic progression and chromosome segregation. PRL phosphatases promote cell migration, invasion, and metastasis, and inhibition of these PTPases has been shown to inhibit proliferation of cancer cells in vitro and tumors in animal models.

By “protein tyrosine phosphatase” or “PTPase” is meant an enzyme that dephosphorylates a tyrosine residue on a protein substrate.

By “dual specificity phosphatase” is meant a protein phosphatase that can dephosphorylate both a tyrosine residue and either a serine or threonine residue on the same protein substrate. Dual specificity phosphatases include

MKP-1, MKP-2, and the cell division cycle phosphatase family (e.g., CDC14, CDC25A, CDC25B, and CDC25C). Dual specificity phosphatases are considered to be protein tyrosine phosphatases.

Protein tyrosine phosphatases include the PRL family (PRL-1, PRL-2, and PRL-3), PTP1B, SHP-1, SHP-2, MKP-1, MKP-2, CDC14, CDC25A, CDC25B, CDC25C, PTPα, and PTP-BL. Protein tyrosine phosphatase biological activities include dephosphorylation of tyrosine residues on substrates. The GenBank Accession Nos. of representative tyrosine phosphatases are provided below.

| Protein | |
|---|---|
| name | GenBank Accession No. |
| PRL-1 | AJ420505, BI222469, U48296 |
| PRL-2 | AF208850, BI552091, L48723 |
| PRL-3 | AF041434, BC003105 |
| PTP1B | AU117677, M33689 |
| SHP-1 | BC002523, BG754792, M77273, BM742181, AF178946 |
| SHP-2 | AU123593, BF515187, BX537632, D13540 |
| MKP-1 | U01669, X68277 |
| MKP-2 | BC014565, U21108, U48807, AL137704 |
| CDC14A | AF000367, AF064102, AF064103 |
| CDC14B | AF023158, AF064104 |
| CDC25A | M81933 |
| CDC25B | M81934, Z68092, AF036233 |
| CDC25C | M34065, Z29077, AJ304504, M34065 |
| PTPalpha | M36033 |
| PTP-BL | D21210, D21209, D21211, U12128 |

Protein Tyrosine Phosphatase Inhibitors

By “protein tyrosine phosphatase inhibitor” is an agent that binds a protein tyrosine phosphatase and inhibits (e.g. by at least 10%, 20%, or 30% or more) the biological activity of that protein tyrosine phosphatase.

Inhibitors of protein tyrosine phosphatases include pentamidine, levamisole, ketoconazole, bisperoxovanadium compounds (e.g., those described in Scrivens et al., Mol. Cancer Ther. 2:1053-1059, 2003, and U.S. Pat. No. 6,642,221), vanadate salts and complexes (e.g., sodium orthovanadate), dephosphatin, dnacin A1, dnacin A2, STI-571, suramin, gallium nitrate, sodium stibogluconate, meglumine antimonate, 2-(2-mercaptoethanol)-3-methyl-1,4-naphthoquinone, 2,5-bis(4-amidinophenyl)furan-bis-O-methylamidoxime, known as DB289 (Immtech), 2,5-bis(4-amidinophenyl)furan (DB75, Immtech), disclosed in U.S. Pat. No. 5,843,980, and compounds described in Pestell et al., Oncogene 19:6607-6612, 2000, Lyon et al., Nat. Rev. Drug Discov. 1:961-976, 2002, Ducruet et al., Bioorg. Med. Chem. 8:1451-1466, 2000, U.S. Patent Application Publication Nos. 2003/0114703, 2003/0144338, and 2003/0161893, and PCT Patent Publication Nos. WO99/46237, WO03/06788 and WO03/070158. Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, and U.S. Patent Application Publication Nos. US 2001/0044468 and US 2002/0019437, and the pentamidine analogs described in U.S. patent application Ser. No. 10/617,424 (see, e.g., Formula (II)). Other protein tyrosine phosphatase inhibitors can be identified, for example, using the methods described in Lazo et al. (Oncol. Res. 13:347-352, 2003), PCT Publication Nos. WO97/40379, WO03/003001, and WO03/035621, and U.S. Pat. Nos. 5,443,962 and 5,958,719.

Other Biological Activity Inhibitors

In addition to reducing biological activity through the use of compounds that bind a mitotic kinesin or protein tyrosine phosphatase, other inhibitors of mitotic kinesin and protein tyrosine phosphatase biological activity can be employed. Such inhibitors include compounds that reduce the amount of target protein or RNA levels and compounds that compete with endogenous mitotic kinesins or protein tyrosine phosphatases for binding partners (e.g., dominant negative proteins).

Dominant Negative Proteins

By “dominant negative” is meant a protein that contains at least one mutation that inactivates its physiological activity such that the expression of this mutant in the presence of the normal or wild type copy of the protein results in inactivation of or reduction of the activity of the normal copy. Thus, the activity of the mutant “dominates” over the activity of the normal copy such that even though the normal copy is present, biological function is reduced. In one example, a dimer of two copies of the protein are required so that even if one normal and one mutated copy are present there is no activity; another example is when the mutant binds to or “soaks up” other proteins that are critical for the function of the normal copy such that not enough of these other proteins are present for activity of the normal copy.

One skilled in the art would know how to make dominant negative mitotic kinesins and protein tyrosine phosphatases. Such dominant negative proteins are described, for example, in Gupta et al., J. Exp. Med., 186:473-478, 1997; Maegawa et al., J. Biol. Chem. 274:30236-30243, 1999; Woodford-Thomas et al., J. Cell Biol. 117:401-414, 1992.

Aurora Kinase Inhibitors

Aurora kinases have been shown to be protein kinases of a new family that regulate the structure and function of the mitotic spindle. One target of Aurora kinases include mitotic kinesins. Aurora kinase inhibitors thus can be used in combination with a compound that reduces protein tyrosine phosphatase biological activity according to a method, composition, or kit of the invention.

There are three classes of aurora kinases: aurora-A, aurora-B and aurora-C. Aurora-A includes AIRK1, DmAurora, HsAurora-2, HsAIK, HsSTK15, CeAIR-1, MmARK1, MmAYK1, MmIAK1 and XIEg2. Aurora-B includes AIRK-2, DmIAL-1, HsAurora-1, HsAIK2, HsAIM-1, HsSTK12, CeAIR-2, MmARK2 and XAIRK2. Aurora-C includes HsAIK3 (Adams, et al., Trends Cell Biol. 11:49-54, 2001).

Aurora kinase inhibitors include VX-528 and ZM447439; others are described, e.g., in U.S. Patent Application Publication No. 2003/0105090 and U.S. Pat. Nos. 6,610,677, 6,593,357, and 6,528,509.

Farnesyltransferase Inhibitors

Farnesyltransferase inhibitors alter the biological activity of PRL phosphatases and thus can be used in combination with a compound that reduces mitotic kinesin activity in a method, composition, or kit of the invention. Farnesyltransferase inhibitors include arglabin, lonafarnib, BAY-43-9006, tipifarnib, perillyl alcohol, FTI-277 and BMS-214662, as well as those compounds described, e.g., in Kohl, Ann. NY Acad. Sci. 886:91-102, 1999, U.S. Patent Application Publication Nos. 2003/0199544, 2003/0199542, 2003/0087940, 2002/0086884, 2002/0049327, and 2002/0019527, U.S. Pat. Nos. 6,586,461 and 6,500,841, and WO03/004489.

Antiproliferative Agents

Antiproliferative agents are described above. Exemplary antiproliferative agents of the invention include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors.

Combination Therapies

In addition to incorporation of an anti-scarring drug combination (or individual component(s) thereof), one or more other pharmaceutically active agents can be incorporated into the present compositions to improve or enhance efficacy. In one aspect, the composition may further include a compound that acts to have an inhibitory effect on pathological processes in or around the treatment site. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

In one aspect, the present invention also provides for the combination of an electrical device (as well as compositions and methods for making electrical devices) that includes an anti-fibrosis (or anti-gliosis) drug combination, or individual component(s) thereof, and an anti-infective agent, which reduces the likelihood of infections.

Infection is a common complication of the implantation of foreign bodies such as, for example, medical devices. Foreign materials provide an ideal site for micro-organisms to attach and colonize. It is also hypothesized that there is an impairment of host defenses to infection in the microenvironment surrounding a foreign material. These factors make medical implants particularly susceptible to infection and make eradication of such an infection difficult, if not impossible, in most cases.

The present invention provides agents (e.g., chemotherapeutic agents) that can be released from a composition, and which have potent antimicrobial activity at extremely low doses. A wide variety of anti-infective agents can be utilized in combination with the present compositions. Suitable anti-infective agents may be readily determined based the assays provided in Example 55. Discussed in more detail below are several representative examples of agents that can be used: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

d) Anthracyclines

In certain embodiments, the anti-infective therapeutic agent is an anthracycline. Anthracyclines have the following general structure, where the R groups may be a variety of organic groups:

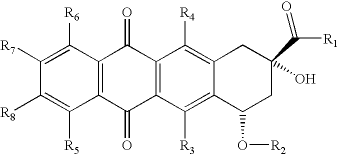

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 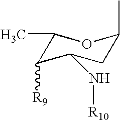
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

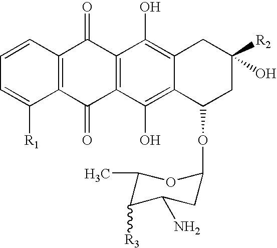

| | R₁ | R₂ | R₃ |
|---|---|---|---|
| Doxorubicin: | OCH₃ | C(O)CH₂OH | OH out of ring |
| | | | plane |
| Epirubicin: | OCH₃ | C(O)CH₂OH | OH in ring plane |
| (4′ epimer of | | | |
| doxorubicin) | | | |
| Dauno- | OCH₃ | C(O)CH₃ | OH out of ring |
| rubicin: | | | plane |
| Idarubicin: | H | C(O)CH₃ | OH out of ring |
| | | | plane |
| Pirarubicin: | OCH₃ | C(O)CH₂OH | 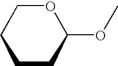 |
| Zorubicin: | OCH₃ | C(CH₃)(═N)NHC(O)C₆H₅ | OH |
| Carubicin: | OH | C(O)CH₃ | OH out of ring |
| | | | plane |

Other suitable anthracyclines are anthramycin, mitoxantrone, menogaril, nogalamycin, aclacinomycin A, olivomycin A, chromomycin A₃, and plicamycin having the structures:

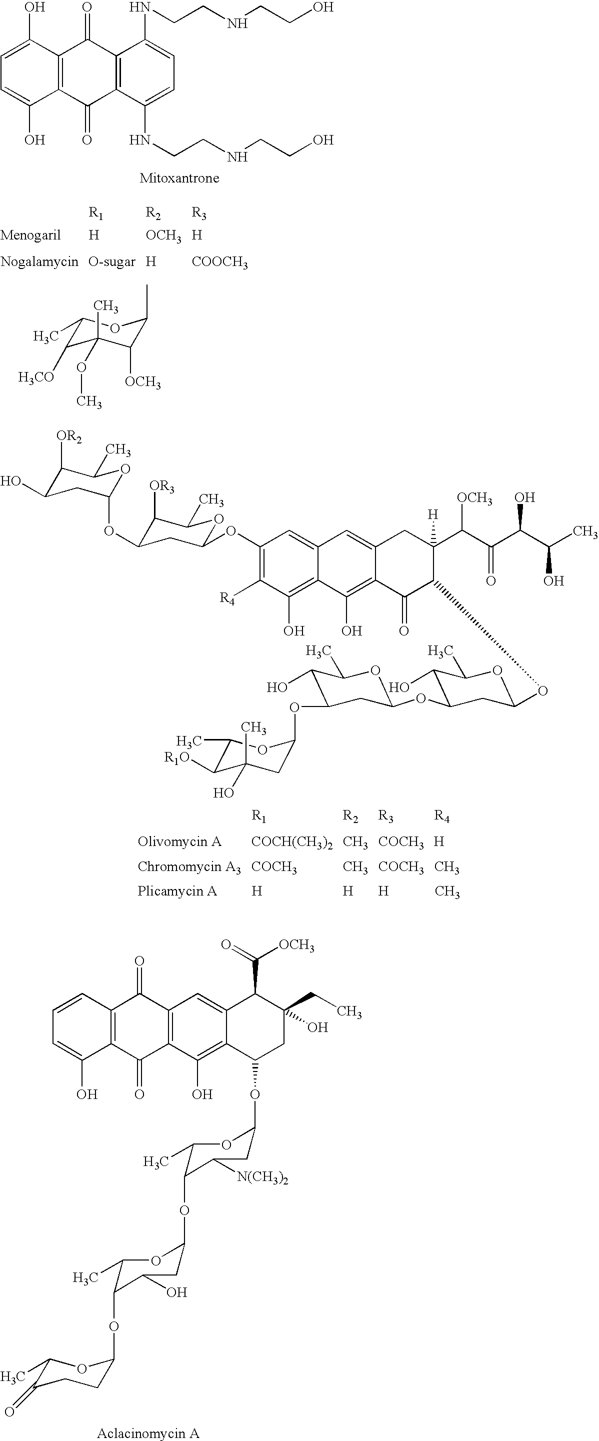

Other representative anthracyclines include, FCE 23762, a doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6): 1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-[2,6-d]deoxy-4-O-(2,3,6-trideoxy-3-amino-α-L-lyxo-hexopyranosyl)-α-L-lyxo-hexopyranosyl]adriamicinone doxorubicin disaccharide analogue (Monteagudo et al., Carbohydr. Res. 300(1):11-16, 1997), 2-pyrrolinodoxorubicin (Nagy et al., Proc. Nat'l Acad. Sci. USA. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8): 1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'Cancer Inst. 80(16):1294-8, 1988), deoxydihydroiodooxorubicin (EPA 275966), adriblastin (Kalishevskaya et al., Vestn. Mosk Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydroxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

e) Fluoropyrimidine Analogues

In another aspect, the anti-infective therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

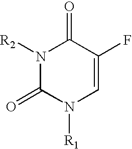

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

B

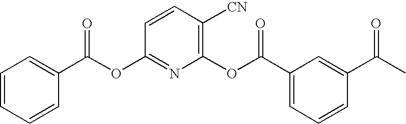

C

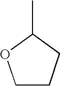

Other suitable fluoropyrimidine analogues include 5-FudR (5-fluoro-deoxyuridine), or an analogue or derivative thereof, including 5-iododeoxyuridine (5-IudR), 5-bromodeoxyuridine (5-BudR), fluorouridine triphosphate (5-FUTP), and fluorodeoxyuridine monophosphate (5-dFUMP). Exemplary compounds have the structures:

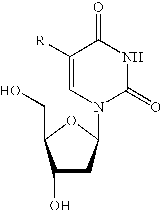

| | |
|---|---|
| 5-Fluoro-2′-deoxyuridine: | R = F |
| 5-Bromo-2′-deoxyuridine: | R '2 Br |
| 5-Iodo-2′-deoxyuridine: | R '2 I |

Other representative examples of fluoropyrimidine analogues include N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3): 144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluoyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

f) Folic Acid Antagonists

In another aspect, the anti-infective therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure: 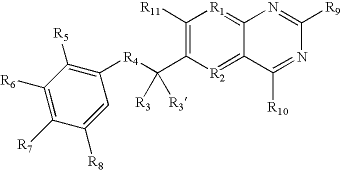
The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 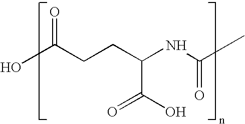
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

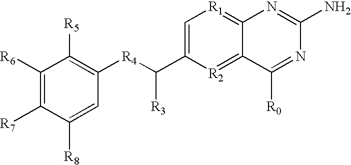

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatexate | NH₂ | N | N | H | CH(CH₂CH₃) | H | H | A (n = 1) | H |
| Trimetrexate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH₃ | N(CH₃) | H | H | A (n = 1) | H |
| Peritrexim | NH₂ | N | C(CH₃) | H | single bond | OCH₃ | H | H | OCH₃ |

A

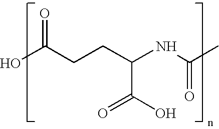

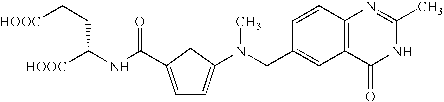

Tomudex

Other representative examples include 6-S-aminoacyloxymethyl mercaptopurine derivatives (Harada et al., Chem. Pharm. Bull. 43(10): 793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7): 1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaminopterin analogues (DeGraw et al., J. Med. Chem. 40(3):370-376, 1997), 5-deazaminopterin and 5,10-dideazaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm. Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl) methotrexate derivatives (Cheung et al., Pteridines 3(1-2):101-2, 1992), biotin methotrexate derivatives (Fan et al., Pteridines 3(1-2): 131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 3](7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-γ-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem., Biol. Clin. Aspects: 659-62, 1986), iodoacetyl lysine methotrexate analogue (Delcamp et al., Chem. Biol. Pteridines, Pteridines Folic Acid Deriv., Proc. Int. Symp. Pteridines Folic Acid Deriv.: Chem., Biol. Clin. Aspects: 807-9, 1986), 2,.omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122(Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly(L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 7](6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220);

These compounds are believed to act as antimetabolites of folic acid.

g) Podophyllotoxins

In another aspect, the anti-infective therapeutic agent is a podophyllotoxin, or a derivative or an analogue thereof. Exemplary compounds of this type are etoposide or teniposide, which have the following structures:

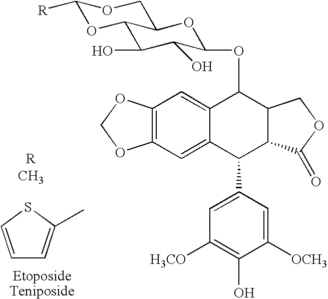

Other representative examples of podophyllotoxins include Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

These compounds are believed to act as topoisomerase II inhibitors and/or DNA cleaving agents.

h) Camptothecins

In another aspect, the anti-infective therapeutic agent is camptothecin, or an analogue or derivative thereof. Camptothecins have the following general structure.

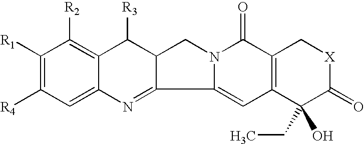

In this structure, X is typically 0, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

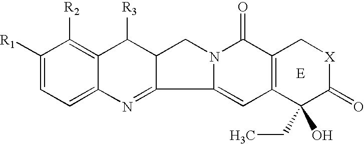

| | R₁ | R₂ | R₃ |
|---|---|---|---|
| Camtothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

i) Hydroxyureas

The anti-infective therapeutic agent of the present invention may be a hydroxyurea. Hydroxyureas have the following general structure:

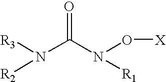

Suitable hydroxyureas are disclosed in, for example, U.S. Pat. No. 6,080,874, wherein R₁ is: 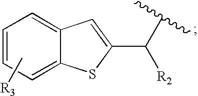
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 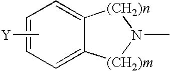
wherein m is 1 or 2, n is 0-2 and Y is an alkyl group.

In one aspect, the hydroxyurea has the structure:

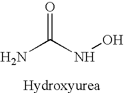

These compounds are thought to function by inhibiting DNA synthesis.

j) Platinum Complexes

In another aspect, the anti-infective therapeutic agent is a platinum compound. In general, suitable platinum complexes may be of Pt(II) or Pt(IV) and have this basic structure: 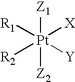
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

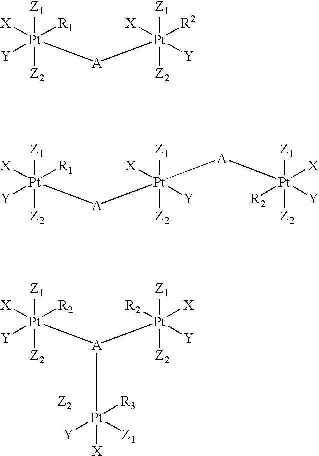

Exemplary platinum compounds are cisplatin, carboplatin, oxaliplatin, and miboplatin having the structures:

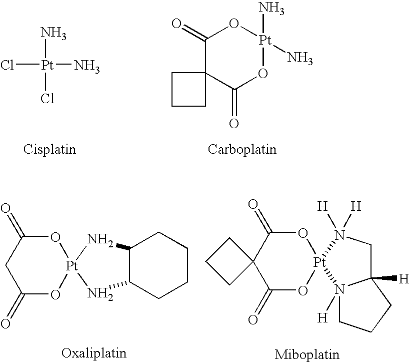

Other representative platinum compounds include (CPA)₂Pt[DOLYM] and (DACH)Pt[DOLYM] cisplatin (Choi et al., Arch. Pharmacal Res. 22(2):151-156, 1999), Cis-[PtCl₂(4,7-H-5-methyl-7-oxo]1,2,4[triazolo[1,5-a]pyrimidine)₂] (Navarro et al., J. Med. Chem. 41(3):332-338, 1998), [Pt(cis-1,4-DACH)(trans-Cl₂)(CBDCA)].½MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356, 1997), Pt(II) . . . Pt(II) (Pt₂-[NHCHN(C(CH₂)(CH₃))]₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-[Pt(OAc)₂I₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-[Pt(NH₃)(4-aminoTEMP-O) {d(GpG)}] (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. [2](1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 3](47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyphenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2): 157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6:443-5, 1987), aliphatic tricarboxylic acid platinum complexes (EPA 185225), and cis-dichloro(amino acid) (tert-butylamine)platinum(II) complexes (Pasini & Bersanetti, Inorg. Chim. Acta 107(4):259-67, 1985). These compounds are thought to function by binding to DNA, i.e., acting as alkylating agents of DNA.

As medical implants are made in a variety of configurations and sizes, the exact dose administered may vary with device size, surface area, design and portions of the implant coated. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the cardiac implant, the preferred anti-infective agents, used alone or in combination, may be administered under the following dosing guidelines:

(a) Anthracyclines. Utilizing the anthracycline doxorubicin as an example, whether applied as a polymer coating, incorporated into the polymers which make up the implant components, or applied without a carrier polymer, the total dose of doxorubicin applied to the implant should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, doxorubicin should be applied to the implant surface at a dose of 0.1 μg/mm²-10 μg/mm². As different polymer and non-polymer coatings may release doxorubicin at differing rates, the above dosing parameters should be utilized in combination with the release rate of the drug from the implant surface such that a minimum concentration of 10⁻⁸-10⁻⁴ M of doxorubicin is maintained on the surface. It is necessary to insure that surface drug concentrations exceed concentrations of doxorubicin known to be lethal to multiple species of bacteria and fungi (i.e., are in excess of 10⁻⁴ M; although for some embodiments lower concentrations are sufficient). In a preferred embodiment, doxorubicin is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers which make up the implant, or applied without a carrier polymer, the total dose of mitoxantrone applied should not exceed 5 mg (range of 0.01 g to 5 mg). In a particularly preferred embodiment, the total amount of drug applied should be in the range of 0.1 μg to 3 mg. The dose per unit area (i.e., the amount of drug as a function of the surface area of the portion of the implant to which drug is applied and/or incorporated) should fall within the range of 0.01 μg-20 μg per mm² of surface area. In a particularly preferred embodiment, mitoxantrone should be applied to the implant surface at a dose of 0.05 μg/mm²-5 μg/mm². As different polymer and non-polymer coatings will release mitoxantrone at differing rates, the above dosing parameters should be utilized in combination with the release rate of the drug from the implant surface such that a minimum concentration of 10⁻⁴-10⁻⁸M of mitoxantrone is maintained. It is necessary to insure that drug concentrations on the implant surface exceed concentrations of mitoxantrone known to be lethal to multiple species of bacteria and fungi (i.e., are in excess of 10⁻⁵ M; although for some embodiments lower drug levels will be sufficient). In a preferred embodiment, mitoxantrone is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines Utilizing the fluoropyrimidine 5-fluorouracil as an example, whether applied as a polymer coating, incorporated into the polymers which make up the implant, or applied without a carrier polymer, the total dose of 5-fluorouracil applied should not exceed 250 mg (range of 1.0 μg to 250 mg). In a particularly preferred embodiment, the total amount of drug applied should be in the range of 100 μg to 25 mg. The dose per unit area (i.e., the amount of drug as a function of the surface area of the portion of the implant to which drug is applied and/or incorporated) should fall within the range of 0.05 μg-200 μg per mm² of surface area. In a particularly preferred embodiment, 5-fluorouracil should be applied to the implant surface at a dose of 0.5 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins Utilizing the podophylotoxin etoposide as an example, whether applied as a polymer coating, incorporated into the polymers which make up the cardiac implant, or applied without a carrier polymer, the total dose of etoposide applied should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, etoposide should be applied to the implant surface at a dose of 0.1 μg/mm²-10 μg/mm². As different polymer and non-polymer coatings will release etoposide at differing rates, the above dosing parameters should be utilized in combination with the release rate of the drug from the implant surface such that a concentration of 10⁻⁴-10⁻⁷ M of etoposide is maintained. It is necessary to insure that surface drug concentrations exceed concentrations of etoposide known to be lethal to a variety of bacteria and fungi (i.e., are in excess of 10⁻⁵ M; although for some embodiments lower drug levels will be sufficient). In a preferred embodiment, etoposide is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

It may be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be utilized to enhance the antibacterial activity of the composition.

In another aspect, an anti-infective agent (e.g., anthracyclines (e.g., doxorubicin or mitoxantrone), fluoropyrimidines (e.g., 5-fluorouracil), folic acid antagonists (e.g., methotrexate and/or podophylotoxins (e.g., etoposide)) can be combined with traditional antibiotic and/or anti-fungal agents to enhance efficacy. The anti-infective agent may be further combined with anti-thrombotic and/or antiplatelet agents (for example, heparin, dextran sulphate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy.

In addition to incorporation of the above-mentioned therapeutic agents (i.e., anti-infective agents or fibrosis-inhibiting drug combinations, or individual component(s) thereof), one or more other pharmaceutically active agents can be incorporated into the present compositions and devices to improve or enhance efficacy. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

Implantable electrical devices and compositions for use with implantable electrical devices may further include an anti-thrombotic agent and/or antiplatelet agent and/or a thrombolytic agent, which reduces the likelihood of thrombotic events upon implantation of a medical implant. Within various embodiments of the invention, a device is coated on one aspect with a composition which inhibits fibrosis (and/or restenosis), as well as being coated with a composition or compound which prevents thrombosis on another aspect of the device. Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulphate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX9065a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein IIb/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

Compositions for use with electrical devices may be or include a hydrophilic polymer gel that itself has anti-thrombogenic properties. For example, the composition can be in the form of a coating that can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

Electrical devices and compositions for use with implantable electrical devices may further include a compound which acts to have an inhibitory effect on pathological processes in or around the treatment site. In certain embodiments, the agent may be selected from one of the following classes of compounds: anti-inflammatory agents (e.g., dexamethasone, cortisone, fludrocortisone, prednisone, prednisolone, 6α-methylprednisolone, triamcinolone, betamethasone, and aspirin); MMP inhibitors (e.g., batimistat, marimistat, TIMP's representative examples of which are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359), cytokine inhibitors (chlorpromazine, mycophenolic acid, rapamycin, 1α-hydroxy vitamin D₃), IMPDH (inosine monophosplate dehydrogenase) inhibitors (e.g., mycophenolic acid, ribaviran, aminothiadiazole, thiophenfurin, tiazofurin, viramidine) (Representative examples are included in U.S. Pat. Nos. 5,536,747; 5,807,876; 5,932,600; 6,054,472; 6,128,582; 6,344,465; 6,395,763; 6,399,773; 6,420,403; 6,479,628; 6,498,178; 6,514,979; 6,518,291; 6,541,496; 6,596,747; 6,617,323; and 6,624,184, U.S. Patent Application Nos. 2002/0040022A1, 2002/0052513 A1, 2002/0055483A1, 2002/0068346 A1, 2002/0111378A1, 2002/0111495 A1, 2002/0123520A1, 2002/0143176A1, 2002/0147160A1, 2002/0161038A1, 2002/0173491A1, 2002/0183315A1, 2002/0193612A1, 2003/0027845 A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901A2, WO 03/047512A2, WO 03/053958A1, WO 03/055447A2, WO 03/059269A2, WO 03/063573A2, WO 03/087071A1, WO 99/001545A1, WO 97/40028A1, WO 97/41211A1, WO 98/40381A1, and WO 99/55663A1), p38 MAP kinase inhibitors (MAPK) (e.g., GW-2286, CGP-52411, BIRB-798, SB220025, RO-320-1195, RWJ-67657, RWJ-68354, SCIO-469) (Representative examples are included in U.S. Pat. Nos. 6,300,347; 6,316,464; 6,316,466; 6,376,527; 6,444,696; 6,479,507; 6,509,361; 6,579,874, and 6,630,485, and U.S. Patent Application Publication Nos. 2001/0044538A1, 2002/0013354A1, 2002/0049220A1, 2002/0103245A1, 2002/0151491A1, 2002/0156114A1, 2003/0018051A1, 2003/0073832 A1, 2003/0130257A1, 2003/0130273A1, 2003/0130319A1, 2003/0139388A1, 2003/0139462A1, 2003/0149031A1, 2003/0166647A1, and 2003/0181411A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2, WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1), and immunomodulatory agents (rapamycin, everolimus, ABT-578, azathioprine azithromycin, analogues of rapamycin, including tacrolimus and derivatives thereof (e.g., EP 0184162B1 and those described in U.S. Pat. No. 6,258,823) and everolimus and derivatives thereof (e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

Other examples of biologically active agents which may be combined with implantable electrical devices according to the invention include tyrosine kinase inhibitors, such as imantinib, ZK-222584, CGP-52411, CGP-53716, NVP-AAK980-NX, CP-127374, CP-564959, PD-171026, PD-173956, PD-180970, SU-0879, and SKI-606; MMP inhibitors such as nimesulide, PKF-241-466, PKF-242-484, CGS-27023A, SAR-943, primomastat, SC-77964, PNU-171829, AG-3433, PNU-142769, SU-5402, and dexlipotam; p38 MAP kinase inhibitors such as include CGH-2466 and PD-98-59; immunosuppressants such as argyrin B, macrocyclic lactone, ADZ-62-826, CCI-779, tilomisole, amcinonide, FK-778, AVE-1726, and MDL-28842; cytokine inhibitors such as TNF-484A, PD-172084, CP-293121, CP-353164, and PD-168787; NFKB inhibitors, such as, AVE-0547, AVE-0545, and IPL-576092; HMGCoA reductase inhibitors, such as, pravestatin, atorvastatin, fluvastatin, dalvastatin, glenvastatin, pitavastatin, CP-83101, U-20685; apoptosis antagonist (e.g., troloxamine, TCH-346 (N-methyl-N-propargyl-10-aminomethyl-dibenzo[b,f)oxepin); and certain caspase inhibitors (e.g., PF-5901 (benzenemethanol, alpha-pentyl-3-(2-quinolinylmethoxy)-), and JNK inhibitor (e.g., AS-602801).

In embodiments, the electrical device may further include an antibiotic (e.g., amoxicillin, trimethoprim-sulfamethoxazole, azithromycin, clarithromycin, amoxicillin-clavulanate, cefprozil, cefuroxime, cefpodoxime, or cefdinir) and/or an anti-fungal agent.

In certain embodiments, a composition comprising a fibrosis-inhibiting drug combination is combined with an agent that can modify metabolism of the agent in vivo to enhance efficacy of the fibrosis-inhibiting agent. One class of therapeutic agents that can be used to alter drug metabolism includes agents capable of inhibiting oxidation of the anti-scarring agent by cytochrome P450 (CYP). In one embodiment, compositions are provided that include a fibrosis-inhibiting drug combination and a CYP inhibitor, which may be combined (e.g., coated) with any of the devices described herein. Representative examples of CYP inhibitors include flavones, azole anti-fungals, macrolide antibiotics, HIV protease inhibitors, and anti-sense oligomers. Devices comprising a combination of a fibrosis-inhibiting drug combination and a CYP inhibitor may be used to treat a variety of proliferative conditions that can lead to undesired scarring of tissue, including intimal hyperplasia, surgical adhesions, and tumor growth.

Within various embodiments of the invention, a device incorporates or is coated on one aspect, portion or surface with a composition which inhibits fibrosis (or gliosis), as well as with a composition or compound which promotes fibrosis on another aspect, portion or surface of the device. Representative examples of agents that promote fibrosis include silk and other irritants (e.g., talc, wool (including animal wool, wood wool, and synthetic wool), talcum powder, copper, metallic beryllium (or its oxides), quartz dust, silica, crystalline silicates), polymers (e.g., polylysine, polyurethanes, poly(ethylene terephthalate), PTFE, poly(alkylcyanoacrylates), and poly(ethylene-co-vinylacetate); vinyl chloride and polymers of vinyl chloride; peptides with high lysine content; growth factors and inflammatory cytokines involved in angiogenesis, fibroblast migration, fibroblast proliferation, ECM synthesis and tissue remodeling, such as epidermal growth factor (EGF) family, transforming growth factor-α (TGF-α), transforming growth factor-(TGF-β (TGF-β-1, TGF-α-2, TGF-α-3, platelet-derived growth factor (PDGF), fibroblast growth factor (acidic—aFGF; and basic—bFGF), fibroblast stimulating factor-1, activins, vascular endothelial growth factor (including VEGF-2, VEGF-3, VEGF-A, VEGF-B, VEGF-C, placental growth factor—PlGF), angiopoietins, insulin-like growth factors (IGF), hepatocyte growth factor (HGF), connective tissue growth factor (CTGF), myeloid colony-stimulating factors (CSFs), monocyte chemotactic protein, granulocyte-macrophage colony-stimulating factors (GM-CSF), granulocyte colony-stimulating factor (G-CSF), macrophage colony-stimulating factor (M-CSF), erythropoietin, interleukins (particularly IL-1, IL-8, and IL-6), tumor necrosis factor-α (TNFα), nerve growth factor (NGF), interferon-α, interferon-β, histamine, endothelin-1, angiotensin II, growth hormone (GH), and synthetic peptides, analogues or derivatives of these factors are also suitable for release from specific implants and devices to be described later. Other examples include CTGF (connective tissue growth factor); inflammatory microcrystals (e.g., crystalline minerals such as crystalline silicates); bromocriptine, methylsergide, methotrexate, chitosan, N-carboxybutyl chitosan, carbon tetrachloride, thioacetamide, fibrosin, ethanol, bleomycin, naturally occurring or synthetic peptides containing the Arg-Gly-Asp (RGD) sequence, generally at one or both termini (see, e.g., U.S. Pat. No. 5,997,895), and tissue adhesives, such as cyanoacrylate and crosslinked poly(ethylene glycol)-methylated collagen compositions. Other examples of fibrosis-inducing agents include bone morphogenic proteins (e.g., BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Of these, BMP-2, BMP-3, BMP-4, BMP-5, BMP-6, and BMP-7 are of particular utility. Bone morphogenic proteins are described, for example, in U.S. Pat. Nos. 4,877,864; 5,013,649; 5,661,007; 5,688,678; 6,177,406; 6,432,919; and 6,534,268 and Wozney, J. M., et al. (1988) Science: 242(4885); 1528-1534.

Other representative examples of fibrosis-inducing agents include components of extracellular matrix (e.g., fibronectin, fibrin, fibrinogen, collagen (e.g., bovine collagen), including fibrillar and non-fibrillar collagen, adhesive glycoproteins, proteoglycans (e.g., heparin sulfate, chondroitin sulfate, dermatan sulfate), hyaluronan, secreted protein acidic and rich in cysteine (SPARC), thrombospondins, tenacin, and cell adhesion molecules (including integrins, vitronectin, fibronectin, laminin, hyaluronic acid, elastin, bitronectin), proteins found in basement membranes, and fibrosin) and inhibitors of matrix metalloproteinases, such as TIMPs (tissue inhibitors of matrix metalloproteinases) and synthetic TIMPs, such as, e.g., marimistat, batimistat doxycycline, tetracycline, minocycline, TROCADE, Ro-1130830, CGS 27023A, and BMS-275291 and analogues and derivatives thereof.

Although the above therapeutic agents have been provided for the purposes of illustration, it may be understood that the present invention is not so limited. For example, although agents are specifically referred to above, the present invention may be understood to include analogues, derivatives and conjugates of such agents. As an illustration, combretastatin A4 may be understood to refer to not only the common chemically available form of combretastatin, but analogues (e.g., combretastatin A2, A3, A5, A6, as noted above) and combretastatin conjugates. In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially.

Dosages

Since neurostimulation devices and cardiac rhythm management devices are made in a variety of configurations and sizes, the exact dose administered may vary with device size, surface area and design. However, certain principles can be applied in the application of this art. Drug dose can be calculated as a function of dose (i.e., amount) per unit area of the portion of the device being coated. Surface area can be measured or determined by methods known to one of ordinary skill in the art. Total drug dose administered can be measured and appropriate surface concentrations of active drug can be determined. Drugs are to be used at concentrations that range from several times more than to 10%, 5%, or even less than 1% of the concentration typically used in a single chemotherapeutic systemic dose application. In certain aspects, the drug is released in effective concentrations for a period ranging from 1-90 days. Regardless of the method of application of the drug to the device, the fibrosis-inhibiting (or gliosis-inhibiting) drug combinations, or individual component(s) thereof, should be administered under the following dosing guidelines:

As described above, electrical devices may be used in combination with a composition that includes an anti-scarring drug combination, or individual component(s) thereof. The total amount (dose) of anti-scarring agent(s) in the drug combinations, or individual component(s) thereof, in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

It should be apparent to one of skill in the art that potentially any anti-scarring drug combination, or individual component(s) thereof, described above may be utilized alone, or in combination, in the practice of this embodiment.

In various aspects, the present invention provides a medical device that comprises an anti-fibrosing (or anti-gliosing) agent listed below in a dosage as set forth above: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, (1) a triazole (e.g., fluconazole or itraconazole) and (2) a aminopyridine (e.g., phenazopyridine (PZP), phenothiazine, dacarbazine, phenelzine); (1) an antiprotozoal (e.g., pentamidine) and (2) a diaminopyridine (e.g., phenazopyridine) or a quaternary ammonium compound (e.g., pentolinium); (1) an aromatic diamidine and (2) one selected from the group consisting of: (a) an antiestrogen, (b) an anti-fungal imidazole, (d) disulfuram, (e) ribavirin, (f) (i) aminopyridine and (ii) phenothiazine, dacarbazine, or phenelzine, (g) (i) a quaternary ammonium compound and (ii) an anti-fungal imidazole, halopnogin, MnSO₄, or ZnCl₂, (h) (i) an antiestrogen and (ii) phenothiazine, cupric chloride, dacarbazine, methoxsalen, or phenelzine, (j) (i) an anti-fungal imidazone and (ii) disulfuram or ribavirin, and (k) an estrogenic compound and (ii) dacarbazine; (1) amphotericin B and (2) dithiocarbamoyl disulfide (e.g., disulfuram); (1) terbinafine and (2) a manganese compound; (1) a tricyclic antidepressant (TCA) (e.g., amoxapine) and (2) a corticosteroid (e.g., prednisolone, glucocorticoid, mineralocorticoid); (1) a tetra-substituted pyrimidopyrimidine (e.g., dipyridamole) and (2) a corticosteroid (e.g., fludrocortisone or prednisolone); (1) a prostaglandin (e.g., alprostadil) and (2) a retinoid (e.g., tretinoin (vitamin A)); (1) an azole (e.g., imidazone or triazole) and (2) a steroid (e.g., corticosteroids including glucocorticoid or mineralocorticoid); (1) a steroid and (2) a prostaglandin, beta-adrenergic receptor ligand, anti-mitotic agent, or microtubule inhibitor; (1) a serotonin norepinephrine reuptake inhibitor (SNRI) or naradrenaline reuptake inhibitor (NARI) and (2) a corticosteroid; (1) a non-steroidal immunophilin-dependent immunosuppressant (NSIDI) (e.g., calcineurin inhibitor including cyclosporin, tacrolimus, ascomycin, pimecrolimus, ISAtx 247) and (2) a non-steroidal immunophilin-dependent immunosuppressant enhancer (NSIDIE) (e.g., selective serotonin reuptake inhibitors, tricyclic antidepressants, phenoxy phenols, anti-histamine, phenothiazines, or mu opioid receptor agonists); (1) an antihistamines and (2) an additional agent selected from corticosteroids, tricyclic or tetracyclic antidepressants, selective serotonin reuptake inhibitors, and steroid receptor modulators; (1) a tricyclic compound and (2) a corticosteroid; (1) an antipsychotic drug (e.g., chlorpromazine) and (2) an antiprotozoal drug (e.g., pentamidine); (1) an antihelmintic drug (e.g., benzimidazole) and (2) an antiprotozoal drug (e.g., pentamidine); (1) ciclopirox and (2) an antiproliferative agent; (1) a salicylanilide (e.g., niclosamide) and (2) an antiproliferative agents; (1) pentamidine or its analogue and (2) chlorpromazine or its analogue; (1) an antihelmintic drug (e.g., alberdazole, mebendazole, oxibendazole) and (2) an antiprotozoal drug (e.g., pentamidine); (1) a dibucaine or amide local anaesthetic related to bupivacaine and (2) a vinca alkaloid; (1) pentamidine, analogue or metabolite thereof and (2) an antiproliferative agent; (1) a triazole (e.g., itraconazole) and (2) an antiarrhythmic agents (e.g., amiodarone, nicardipine or bepridil); (1) an azole and (2) an HMG-CoA reductase inhibitor; a phenothiazine conjugate (e.g., a conjugate of phenothiazine and an antiproliferative agent; (1) phenothiazine and (2) an antiproliferative agent; (1) a kinesin inhibitor (e.g., phenothiazine, analog or metabolite) and (2) an antiproliferative agent (e.g., Group A and Group B antiproliferative agents); (1) an agent that reduces the biological activity of a mitotic kinesin (e.g., chlorpromazine) and (2) an agent that reduces the biological activity of protein tyrosine phosphatase.

The drug dose administered from the present compositions will depend on a variety of factors, including the type of formulation, the location of the treatment site, and the type of condition being treated, as well as the surface area of the device. However, certain principles can be applied in the application of this art. Drug dose can be calculated as a function of dose per unit area (of the treatment site), wherein the total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring or anti-gliosis drug combination, or individual component(s) thereof, is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In certain embodiments, the drug is released in effective concentrations for a period ranging from 1-90 days. It should be understood in certain embodiments that within the drug combination, one drug may be released at a different rate and/or for a different amount of time than the other drug(s).

The exemplary anti-fibrosing or anti-gliosis drug combinations or individual components thereof should be administered under the following dosing guidelines. The total amount (dose) of anti-scarring or anti-gliosis agent(s) in the drug combinations or compositions that comprise the drug combinations can be in the range of about 0.01 μg-10 μg, or 10 μg-100 μg, or 100 μg-1000 μg, or 1 mg-10 mg, or 10 mg-250 mg, or 250 mg-1000 mg, or 1000 mg-2500 mg. The dose (amount) of anti-scarring or anti-gliosis agent(s) per unit area of surface to which the agent is applied may be in the range of about 0.01 μg/mm²-1 μg/mm², or 1 μg/mm²-10 μg/mm², or 10 μg/mm²-250 μg/mm², or 250 μg/mm²-1000 μg/mm², or 1000 μg/mm²-2500 μg/mm².

Provided below are exemplary drug combinations and dosage ranges for various anti-scarring and/or anti-gliosis drug combinations or individual components thereof that can be used in conjunction with devices in accordance with the invention.

Exemplary anti-fibrotic drug combinations for description of dosing include, but are not limited to amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof: total dose of each drug within the combination not to exceed 500 mg (range of 0.1 μg to 500 mg; preferred 1 μg to 200 mg). Dose per unit area of 0.01 μg/mm² to 200 μg/mm²; preferred dose of 0.1 μg/mm² to 100 μg/mm². Minimum concentration of 10⁻⁸ to 10⁻⁴M of agent is to be maintained on the implant or barrier surface. Molar ratio of each drug in the combination is to be within the range of 1:1 to 1:1000. Molar ratios within this range may include but are not limited to 1:5, 1:10, 1:15, 1:20, 1:30, 1:50, 1:75, 1:100, 1:200, 1:500, 1:1000. Note that molar ratios may also lie between the ratios stated above.

C. Delivery of Anti-Scarring Drug Combinations, or Individual Components Thereof, and Generating Electrical Devices that Comprise Anti-Scarring Drug Combinations or Individual Components Thereof

In the practice of this invention, drug-coated or drug-impregnated implants and medical devices are provided which inhibit fibrosis (or gliosis) in and around the device, lead and/or electrode of neurostimulation or cardiac rhythm management (CRM) devices. Within various embodiments, fibrosis (or gliosis) is inhibited by local, regional or systemic release of specific pharmacological agents that become localized to the tissue adjacent to the device or implant. There are numerous neurostimulation and CRM devices where the occurrence of a fibrotic (or gliotic) reaction may adversely affect the functioning of the device or the biological problem for which the device was implanted or used. Typically, fibrotic (or gliotic) encapsulation of the electrical lead (or the growth of fibrous/glial tissue between the lead and the target nerve tissue) slows, impairs, or interrupts electrical transmission of the impulse from the device to the tissue. This can cause the device to function suboptimally or not at all, or can cause excessive drain on battery life as increased energy is required to overcome the electrical resistance imposed by the intervening scar (or glial) tissue.

Anti-scarring drug combinations (or individual components) of the present invention may be delivered to a site of need (e.g., in and around neurostimulation or cardiac rhythm management devices) in various manners. For instance, in certain embodiments, devices coated or impregnated with an anti-scarring drug combination, or individual component(s) thereof, are provided in and around the implantable device. Within other embodiments, fibrosis is inhibited by local, regional or systemic release of anti-scarring drug combinations, or individual component(s) thereof, that become localized to the tissue adjacent to the device. In certain other embodiments, anti-scarring drug combinations, or individual component(s), may be used to infiltrate a tissue surrounding a device. In certain embodiments, anti-scarring drug combinations, or individual component(s) thereof, are in sustained release preparations.

Individual components of drug combinations may be delivered to a site of treatment together or separately. For instance, in certain embodiments, individual components are combined to form drug combinations before being delivered to a site of treatment. In certain other embodiments, individual components are delivered separately to a site of treatment and combine in situ to become drug combinations. In such embodiments, individual components may be delivered sequentially via a same delivery method (e.g., infiltrating tissue surrounding a device that will be, or is, or has been, implanted), or via different delivery methods (e.g., infiltrating tissue surrounding a device that will be, or is, or has been, implanted with one component, where the device is coated or otherwise combined with another component).

There are numerous methods available for optimizing delivery of the fibrosis-inhibiting (or gliosis-inhibiting) drug combinations, or individual component(s) thereof, to the site of the intervention and several of these are described below.

1) Delivery of Anti-Scarring Drug Combinations, or Individual Component(s) Thereof, Via Electrical Devices and Preparing Electrical Devices that Comprise Fibrosis-Inhibiting (or Gliosis-Inhibiting) Drug Combinations, or Individual Component(s) Thereof

Medical devices or implants of the present invention are coated with, or adapted to release an agent which inhibits fibrosis (or gliosis) on the surface of, or around, the neurostimulator or CRM device, lead and/or electrode. In one aspect, the present invention provides electrical devices that include an anti-scarring (or anti-gliotic) drug composition, or individual component(s) thereof, or a composition that includes an anti-scarring (or anti-gliotic) drug combination, or individual component(s) thereof, such that the overgrowth of granulation (or gliotic) tissue is inhibited or reduced.

Methods for incorporating fibrosis-inhibiting (or gliosis-inhibiting) drug combinations, or individual component(s) thereof, onto or into CRM or neurostimulator devices include: (a) directly affixing to the device, lead and/or the electrode a composition that includes a fibrosis-inhibiting (or gliosis-inhibiting) drug combination, or individual component(s) thereof (e.g., by either a spraying process or dipping process as described below, with or without a carrier); (b) directly incorporating into the device, lead and/or the electrode a composition that includes a fibrosis-inhibiting (or gliosis-inhibiting) drug combination, or individual component(s) thereof (e.g., by either a spraying process or dipping process as described below, with or without a carrier); (c) by coating the device, lead and/or the electrode with a substance such as a hydrogel which may in turn absorb the fibrosis-inhibiting (or gliosis-inhibiting) drug combination, or individual component(s) thereof; (d) by interweaving into the device, lead and/or electrode structure a thread (or the polymer itself formed into a thread) coated by a fibrosis-inhibiting (or gliosis-inhibiting) drug combination, or individual component(s) thereof; (e) by inserting the device, lead and/or the electrode into a sleeve or mesh which is comprised of, or coated with, a fibrosis-inhibiting (or gliosis-inhibiting) drug combination, or individual component(s) thereof; (f) constructing the device, lead and/or the electrode itself (or a portion of the device and/or the electrode) with a fibrosis-inhibiting (or gliosis-inhibiting) drug combination, or individual component(s) thereof; or (g) by covalently binding the fibrosis-inhibiting (or gliosis-inhibiting) drug combination, or individual component(s) thereof, directly to the device, lead and/or electrode surface or to a linker (small molecule or polymer) that is coated or attached to the device surface. For these devices, leads and electrodes, the coating process can be performed in such a manner as to: (a) coat the non-electrode portions of the lead or device; (b) coat the electrode portion of the lead; (c) coat the sensor part of the lead; or (d) coat all or parts of the entire device with the composition that includes a fibrosis-inhibiting (or gliosis-inhibiting) drug combination, or individual component(s) thereof. In addition to, or alternatively, the fibrosis-inhibiting (or gliosis-inhibiting) drug combination, or individual component(s) thereof, can be mixed with the materials that are used to make the device, lead and/or electrode such that the fibrosis-inhibiting (or gliosis-inhibiting) drug combination, or individual component(s) thereof, is incorporated into the final product.

In addition to, or as an alternative to, incorporating a fibrosis-inhibiting (or gliosis-inhibiting) drug combination, or individual component(s) thereof, onto or into the CRM or neurostimulation device, the fibrosis-inhibiting (or gliosis-inhibiting) drug combination, or individual component(s) thereof, can be applied directly or indirectly to the tissue adjacent to the CRM or neurostimulator device (preferably near the electrode-tissue interface). This can be accomplished by applying the fibrosis-inhibiting (or gliosis-inhibiting) drug combination, or individual component(s) thereof, with or without a polymeric, non-polymeric, or secondary carrier: (a) to the lead and/or electrode surface (e.g., as an injectable, paste, gel or mesh) during the implantation procedure); (b) to the surface of the tissue (e.g., as an injectable, paste, gel, in situ forming gel or mesh) prior to, immediately prior to, or during, implantation of the CRM or neurostimulation device, lead and/or electrode; (c) to the surface of the lead and/or electrode and/or the tissue surrounding the implanted lead and/or electrode (e.g., as an injectable, paste, gel, in situ forming gel or mesh) immediately after to the implantation of the CRM or neurostimulation device, lead and/or electrode; (d) by topical application of the anti-fibrosis (or anti-gliosis) drug combination, or individual component(s) thereof, into the anatomical space where the CRM or neurostimulation device, lead and/or electrode may be placed (particularly useful for this embodiment is the use of polymeric carriers which release the fibrosis-inhibiting (or gliosis-inhibiting) drug combination, or individual component(s) thereof, over a period ranging from several hours to several weeks—fluids, suspensions, emulsions, microemulsions, microspheres, pastes, gels, microparticulates, sprays, aerosols, solid implants and other formulations which release the agent can be delivered into the region where the device may be inserted); (e) via percutaneous injection into the tissue surrounding the device, lead and/or electrode as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

In another embodiment, the fibrosis-inhibiting (or gliosis-inhibiting) drug combinations, or individual component(s) thereof, can be coated onto the entire device or a portion of the device. In certain embodiments, the drug combination, or individual component(s) thereof, is present as part of a coating on a surface of the CRM or neurostimulation device, lead and/or electrode. The coating may partially cover or may completely cover the surface of the electrical device, lead and/or electrode. Further, the coating may directly or indirectly contact the electrical device, lead and/or electrode. For example, the CRM or neurostimulation device, lead and/or electrode may be coated with a first coating and then coated with a second coating that includes the anti-scarring (or gliosis-inhibiting) drug combination, or individual component(s) thereof.

CRM and neurostimulation devices, leads and/or electrodes may be coated using a variety of coating methods, including dipping, spraying, painting, vacuum deposition, or any other method known to those of ordinary skill in the art.

As described above, the anti-fibrosing (or anti-gliotic) drug combination, or individual component(s) thereof, can be coated onto the appropriate CRM or neurostimulation device, lead and/or electrode using the polymeric coatings described below. In addition to the coating compositions and methods described below, there are various other coating compositions and methods that are known in the art. Representative examples of these coating compositions and methods are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. Patent Application Publication Nos. 2002/0146581, 2003/0129130, 2001/0026834; 2003/0190420; 2001/0000785; 2003/0059631; 2003/0190405; 2002/0146581; 2003/020399; 2001/0026834; 2003/0190420; 2001/0000785; 2003/0059631; 2003/0190405; and 2003/020399; and PCT Publication Nos. WO 02/055121; WO 01/57048; WO 01/52915; and WO 01/01957.

In yet another aspect, anti-scarring (or anti-gliosis) drug combinations, or individual component(s) thereof, may be located within pores or voids of the electrical device, lead and/or electrode. For example, a CRM or neurostimulation device, lead and/or electrode may be constructed to have cavities (e.g., divets or holes), grooves, lumen(s), pores, channels, and the like, which form voids or pores in the body of the device, lead and/or electrode. These voids may be filled (partially or completely) with a fibrosis-inhibiting (or gliosis-inhibiting) drug combination, or individual component(s) thereof, or a composition that comprises a fibrosis-inhibiting (or gliosis-inhibiting) drug combination, or individual component(s) thereof.

Within another aspect of the invention, the biologically active agent can be delivered with non-polymeric agents. These non-polymeric agents can include sucrose derivatives (e.g., sucrose acetate isobutyrate, sucrose oleate), sterols such as cholesterol, stigmasterol, beta-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydroxyapatite, zeolites, and combinations and mixtures thereof.

Representative examples of patents relating to non-polymeric delivery systems and their preparation include U.S. Pat. Nos. 5,736,152; 5,888,533; 6,120,789; 5,968,542; and 5,747,058.

The fibrosis-inhibiting (or gliosis-inhibiting) drug combination, or individual component(s) thereof, may be delivered as a solution. The fibrosis-inhibiting (or gliosis-inhibiting) drug combination, or individual component(s) thereof, can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, CMC, and the like). In another aspect of the invention, the solution can include a biocompatible solvent, such as ethanol, DMSO, glycerol, PEG-200, PEG-300 or NMP.

Within another aspect of the invention, the fibrosis-inhibiting (or gliosis-inhibiting) drug combination, of individual component(s) thereof, can further comprise a secondary carrier. The secondary carrier can be in the form of microspheres (e.g., PLGA, PLLA, PDLLA, PCL, gelatin, polydioxanone, poly(alkylcyanoacrylate), nanospheres (e.g., PLGA, PLLA, PDLLA, PCL, gelatin, polydioxanone, poly(alkylcyanoacrylate)), liposomes, emulsions, microemulsions, micelles (e.g., SDS, block copolymers of the form X—Y, X—Y—X or Y—X—Y where X is a poly(alkylene oxide) or alkyl ether thereof (e.g., poly(ethylene glycol), methoxy poly(ethylene glycol), poly(propylene glycol), block copolymers of poly(ethylene oxide) and poly(propylene oxide) [e.g., PLURONIC and PLURONIC R polymers (BASF)]) and Y is a polyester where the polyester can comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLGA, PLLA, PDLLA, PCL polydioxanone)), zeolites or cyclodextrins.

Within another aspect of the invention, compositions comprising a fibrosis-inhibiting (or gliosis-inhibiting) drug combination, or individual component(s) thereof, and a secondary carrier can be a) incorporated directly into, or onto, the CRM or neurostimulation device, lead and/or electrode, b) incorporated into a solution, c) incorporated into a gel or viscous solution, d) incorporated into the composition used for coating the device, lead and/or electrode, or e) incorporated into, or onto, the device, lead and/or electrode following coating of the device, lead and/or electrode with a coating composition.

For example, PLGA microspheres loaded with a fibrosis-inhibiting (or gliosis-inhibiting) drug combination, or individual component(s) thereof, may be incorporated into a polyurethane coating solution which is then coated onto the device, lead and/or electrode.

In yet another example, the device, lead and/or electrode can be coated with a polyurethane and then allowed to partially dry such that the surface is still tacky. A particulate form of the fibrosis-inhibiting (or gliosis-inhibiting) drug combination, or individual component(s) thereof, or fibrosis-inhibiting (or gliosis-inhibiting) drug combination (or individual component(s) thereof)/secondary carrier can then be applied to all or a portion of the tacky coating after which the device is dried.

In yet another example, the device, lead and/or electrode can be coated with one of the coatings described above. A thermal treatment process can then be used to soften the coating, after which the fibrosis-inhibiting (or gliosis-inhibiting) drug combination, or individual component(s) thereof, or the fibrosis-inhibiting (or gliosis-inhibiting) drug combination (or individual component(s) thereof)/secondary carrier is applied to the entire device, lead and/or electrode or to a portion of the device, lead and/or electrode (e.g., outer surface).

Within another aspect of the invention, the coated CRM or neurostimulation device, lead and/or electrode which inhibits or reduces an in vivo fibrotic (or gliotic) reaction is further coated with a compound or compositions which delay the release of and/or activity of the fibrosis-inhibiting (or gliosis-inhibiting) drug combination, or individual component(s) thereof. Representative examples of such agents include biologically inert materials such as gelatin, PLGA/MePEG film, PLA, polyurethanes, silicone rubbers, surfactants, lipids, or polyethylene glycol, as well as biologically active materials such as heparin or heparin quaternary amine complexes (e.g., heparin-benzalkonium chloride complex) (e.g., to induce coagulation).

For example, in one embodiment of the invention the active agent on the device, lead and/or electrode is top-coated with a physical barrier. Such barriers can include non-degradable materials or biodegradable materials such as gelatin, PLGA/MePEG film, PLA, or polyethylene glycol among others. In one embodiment, the rate of diffusion of the therapeutic agent in the barrier coat is slower that the rate of diffusion of the therapeutic agent in the coating layer. In the case of PLGA/MePEG, once the PLGA/MePEG becomes exposed to the blood or body fluids, the MePEG may dissolve out of the PLGA, leaving channels through the PLGA to an underlying layer containing the fibrosis-inhibiting (or gliosis-inhibiting) drug combination, or individual component(s) thereof, which then can then diffuse into the tissue and initiate its biological activity.

In another embodiment of the invention, for example, a particulate form of the active agent may be coated onto the CRM or neurostimulation device, lead and/or electrode using a polymer (e.g., PLG, PLA, polyurethane). A second polymer that dissolves slowly or degrades (e.g., MePEG-PLGA or PLG) and that does not contain the active agent may be coated over the first layer. Once the top layer dissolves or degrades, it exposes the under coating which allows the active agent to be exposed to the treatment site or to be released from the coating.

Within another aspect of the invention, the outer layer of the coating of a coated CRM or neurostimulation device, lead and/or electrode which inhibits an in vivo fibrotic (or gliotic) response is further treated to crosslink the outer layer of the coating. This can be accomplished by subjecting the coated device, lead and/or electrode to a plasma treatment process. The degree of crosslinking and nature of the surface modification can be altered by changing the RF power setting, the location with respect to the plasma, the duration of treatment as well as the gas composition introduced into the plasma chamber.

Protection of a biologically active surface can also be utilized by coating the CRM or neurostimulator device, lead and/or electrode surface with an inert molecule that prevents access to the active site through steric hindrance, or by coating the surface with an inactive form of the fibrosis-inhibiting (or gliosis-inhibiting) drug combination, or individual component(s) thereof, which is later activated. For example, the device, lead and/or electrode can be coated with an enzyme, which causes either release of the fibrosis-inhibiting (or gliosis-inhibiting) drug combination, or individual component(s) thereof, or activates the fibrosis-inhibiting (or gliosis-inhibiting) drug combination, or individual component(s) thereof.

Another example of a suitable CRM or neurostimulation device, lead and/or electrode surface coating includes an anticoagulant such as heparin or heparin quaternary amine complexes (e.g., heparin-benzalkonium chloride complex), which can be coated on top of the fibrosis-inhibiting (or gliosis-inhibiting) drug combination, or individual component(s) thereof, this may also be useful during transvenous placement of pacemaker or ICD leads to prevent clotting. The presence of the anticoagulant delays coagulation. As the anticoagulant dissolves away, the anticoagulant activity may stop, and the newly exposed fibrosis-inhibiting (or gliosis-inhibiting) drug combination, or individual component(s) thereof, may inhibit or reduce fibrosis (or gliosis) from occurring in the adjacent tissue or coating the device, lead and/or electrode.

In another aspect, the CRM or neurostimulation device, lead and/or electrode can be coated with an inactive form of the fibrosis-inhibiting (or gliosis-inhibiting) drug combination, or individual component(s) thereof, which is then activated once the device is deployed. Such activation may be achieved by injecting another material into the treatment area after the device, lead and/or electrode (as described below) is implanted or after the fibrosis-inhibiting (or gliosis-inhibiting) drug combination, or individual component(s) thereof, has been administered to the treatment area (via injections, spray, wash, drug delivery catheters or balloons). In this aspect, the device, lead and/or electrode may be coated with an inactive form of the fibrosis-inhibiting (or gliosis-inhibiting) drug combination, or individual component(s) thereof. Once the device, lead and/or electrode is implanted, the activating substance is injected or applied into, or onto, the treatment site where the inactive form of the fibrosis-inhibiting (or gliosis-inhibiting) drug combination, or individual component(s) thereof, has been applied.

One example of this method includes coating a CRM or neurostimulation device, lead and/or electrode with a biologically active fibrosis-inhibiting (or gliosis-inhibiting) drug combination, or individual component(s) thereof, as described herein. The coating containing the active fibrosis-inhibiting (or gliosis-inhibiting) drug combination, or individual component(s) thereof, may then be covered with polyethylene glycol and these two substances may then be bonded through an ester bond using a condensation reaction. Prior to the deployment of the device, lead and/or electrode, an esterase is injected into the tissue around the outside of the device (lead or electrode), which can cleave the bond between the ester and the fibrosis-inhibiting (or gliosis-inhibiting) therapeutic agent, allowing the drug combination, or individual component(s) thereof, to initiate fibrosis (or gliosis) inhibition.

The devices and compositions of the invention may include one or more additional ingredients and/or therapeutic agents, such as surfactants (e.g., PLURONICS, such as F-127, L-122, L-101, L-92, L-81, and L-61), anti-inflammatory agents (e.g., dexamethasone or aspirin), anti-thrombotic agents (e.g., heparin, high activity heparin, heparin quaternary amine complexes (e.g., heparin benzalkonium chloride complex)), anti-infective agents (e.g., 5-fluorouracil, triclosan, rifamycim, and silver compounds), preservatives, anti-oxidants and/or anti-platelet agents.

Within certain embodiments of the invention, the device or therapeutic composition can also comprise radio-opaque, echogenic materials and magnetic resonance imaging (MRI) responsive materials (i.e., MRI contrast agents) to aid in visualization of the composition under ultrasound, fluoroscopy and/or MRI. For example, a composition may be echogenic or radiopaque (e.g., made with echogenic or radiopaque with materials such as powdered tantalum, tungsten, barium carbonate, bismuth oxide, barium sulfate, metrazimide, iopamidol, iohexyl, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). For visualization under MRI, contrast agents (e.g., gadolinium (III) chelates or iron oxide compounds) may be incorporated into the composition. In some embodiments, a medical device may include radio-opaque or MRI visible markers (e.g., bands) that may be used to orient and guide the device during the implantation procedure.

The devices may, alternatively, or in addition, be visualized under visible light, using fluorescence, or by other spectroscopic means. Visualization agents that can be included for this purpose include dyes, pigments, and other colored agents. In certain embodiments, the composition may further include a colorant to improve visualization of the composition in vivo and/or ex vivo. Frequently, compositions can be difficult to visualize upon delivery into a host, especially at the margins of an implant or tissue. A coloring agent can be incorporated into a composition to reduce or eliminate the incidence or severity of this problem. The coloring agent provides a unique color, increased contrast, or unique fluorescence characteristics to the composition. In certain embodiments, a composition is provided that includes a colorant such that it is readily visible (under visible light or using a fluorescence technique) and easily differentiated from its implant site. In another aspect, a colorant can be included in a liquid or semi-solid composition. For example, a single component of a two-component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat-soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan.

In certain embodiments, the devices and compositions of the present invention include one or more coloring agents, also referred to as dyestuffs, which may be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In certain embodiments, the devices compositions of the present invention include one or more preservatives or bacteriostatic agents present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In certain embodiments, the compositions of the present invention include one or more bactericidal (also known as bacteriacidal) agents.

In certain embodiments, the devices and compositions of the present invention include one or more antioxidants, present in an effective amount. Examples of the antioxidant include sulfites, alpha-tocopherol and ascorbic acid.

Within certain aspects of the present invention, the therapeutic composition should be biocompatible, and release one or more fibrosis-inhibiting (or gliosis-inhibiting) agents over a period of several hours, days, or, months. As described above, “release of an agent” refers to any statistically significant presence of the agent, or a subcomponent thereof, which has disassociated from the compositions. The compositions of the present invention may release the anti-scarring agent(s) at one or more phases, the one or more phases having similar or different performance (e.g., release) profiles. The therapeutic agent(s) may be made available to the tissue at amounts which may be sustainable, intermittent, or continuous; in one or more phases; and/or rates of delivery; effective to reduce or inhibit any one or more components of fibrosis (or scarring) (or gliosis), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Thus, release rate may be programmed to impact fibrosis (or scarring) by releasing an anti-scarring drug combination, or individual component(s) thereof, at a time such that at least one of the components of fibrosis (or gliosis) is inhibited or reduced. Moreover, the predetermined release rate may reduce agent loading and/or concentration as well as potentially providing minimal drug washout and thus increases efficiency of drug effect. The anti-scarring agents described herein may perform one or more functions, including inhibiting the formation of new blood vessels (angiogenesis), inhibiting the migration and proliferation of connective tissue cells (such as fibroblasts or smooth muscle cells), inhibiting the deposition of extracellular matrix (ECM), and inhibiting remodeling (maturation and organization of the fibrous tissue). In one embodiment, the rate of release may provide a sustainable level of the anti-scarring agent to the susceptible tissue site. In another embodiment, the rate of release is substantially constant. The rate may decrease and/or increase over time, and it may optionally include a substantially non-release period. The release rate may comprise a plurality of rates. In an embodiment, the plurality of release rates may include rates selected from the group consisting of substantially constant, decreasing, increasing, and substantially non-releasing.

The total amount of anti-scarring agent made available on, in or near the device may be in an amount ranging from about 0.01 μg (micrograms) to about 2500 mg (milligrams). Generally, the anti-scarring agent may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

The surface amount of anti-scarring agent on, in or near the device may be in an amount ranging from less than 0.01 μg to about 250 μg per mm² of device surface area. Generally, the anti-scarring agent may be in the amount ranging from less than 0.01 μg per mm²; or from 0.01 μg to about 10 μg per mm²; or from 10 μg to about 250 μg per mm².

The anti-scarring agent that is on, in or near the device may be released from the composition in a time period that may be measured from the time of implantation, which ranges from about less than 1 day to about 180 days. Generally, the release time may also be from about less than 1 day to about 7 days; from 7 days to about 14 days; from 14 days to about 28 days; from 28 days to about 56 days; from 56 days to about 90 days; from 90 days to about 180 days.

The amount of anti-scarring agent released from the composition as a function of time may be determined based on the in vitro release characteristics of the agent from the composition. The in vitro release rate may be determined by placing the anti-scarring agent within the composition or device in an appropriate buffer such as 0.1M phosphate buffer (pH 7.4)) at 37° C. Samples of the buffer solution are then periodically removed for analysis by HPLC, and the buffer is replaced to avoid any saturation effects.

Based on the in vitro release rates, the release of anti-scarring agent per day may range from an amount ranging from about 0.01 μg (micrograms) to about 2500 mg (milligrams). Generally, the anti-scarring agent that may be released in a day may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the anti-scarring agent is made available to the susceptible tissue site in a programmed, sustained, and/or controlled manner which results in increased efficiency and/or efficacy. Further, the release rates may vary during either or both of the initial and subsequent release phases. There may also be additional phase(s) for release of the same substance(s) and/or different substance(s).

Further, therapeutic compositions and devices of the present invention should preferably have a stable shelf-life of at least several months and be capable of being produced and maintained under sterile conditions. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E.I. DuPont De Nemours and Company, Wilmington, Del.).

In certain embodiments, the compositions and devices of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, TEFLON, silicone, and gray-butyl rubber.

In one embodiment, the product containers can be thermoformed plastics.

In another embodiment, a secondary package can be used for the product. In another embodiment, product can be in a sterile container that is placed in a box that is labeled to describe the contents of the box.

Coating of CRM or Neurostimulation Devices, Leads and Electrodes with Fibrosis-Inhibiting (or Gliosis-Inhibiting) Drug Combinations or Individual Component(s) Thereof

As described below, a range of polymeric and non-polymeric materials can be used to incorporate the fibrosis-inhibiting (or gliosis-inhibiting) drug combination, or individual component(s) thereof, onto or into an electrical device, lead or electrode. Coating the device, lead and/or electrode with these compositions containing the fibrosis-inhibiting (or gliosis-inhibiting) drug combination, or individual component(s) thereof, or with only the fibrosis-inhibiting (or gliosis-inhibiting) drug combination, or individual component(s) thereof, is one process that can be used to incorporate the fibrosis-inhibiting (or gliosis-inhibiting) drug combination, or individual component(s) thereof, into or onto the device, lead and/or electrode.

a) Dip Coating

Dip coating is an example of coating process that can be used to associate the fibrosis-inhibiting (or gliosis-inhibiting) drug combination, or individual component(s) thereof, with the device, lead and/or electrode. In one embodiment, the fibrosis-inhibiting (or gliosis-inhibiting) drug combination, or individual component(s) thereof, is dissolved in a solvent for the fibrosis-inhibiting (or gliosis-inhibiting) drug combination, or individual component(s) thereof, and is then coated onto the device, lead and/or electrode.

Fibrosis-Inhibiting (or Gliosis-Inhibiting) Drug Combination, or Individual Components Thereof with an Inert Solvent

In one embodiment, the solvent is an inert solvent for the device, lead or electrode such that the solvent does not dissolve the medical device, lead or electrode to any great extent and is not absorbed by the device, lead or electrode to any great extent. The device, lead or electrode can be immersed, either partially or completely, in the fibrosis-inhibiting (or gliosis-inhibiting) drug combination (or individual component(s) thereof)/solvent solution for a specific period of time. The rate of immersion into the fibrosis-inhibiting (or gliosis-inhibiting) drug combination (or individual component(s) thereof)/solvent solution can be altered (e.g., 0.001 cm per sec to 50 cm per sec). The device, lead and/or electrode can then be removed from the solution. The rate at which the device, lead or electrode is withdrawn from the solution can be altered (e.g., 0.001 cm per sec to 50 cm per sec). The coated device, lead or electrode can be air-dried. The dipping process can be repeated one or more times depending on the specific application, where higher repetitions generally increase the amount of agent that is coated onto the device, lead or electrode. The device, lead or electrode can be dried under vacuum to reduce residual solvent levels. This process will result in the fibrosis-inhibiting (or gliosis-inhibiting) drug combination, or individual component(s) thereof, being coated on the surface of the device.

Fibrosis-Inhibiting (or Gliosis-Inhibiting) Drug Combination, or Individual Component(s) Thereof with a Swelling Solvent

In one embodiment, the solvent is one that will not dissolve the CRM or neurostimulation device, lead or electrode but will be absorbed by the device, lead or electrode. In certain cases, these solvents can swell the device, lead or electrode to some extent. The device, lead or electrode can be immersed, either partially or completely, in the fibrosis-inhibiting (or gliosis-inhibiting) drug combination (or individual component(s) thereof)/solvent solution for a specific period of time (seconds to days). The rate of immersion into the fibrosis-inhibiting (or gliosis-inhibiting) drug combination (or individual component(s) thereof)/solvent solution can be altered (e.g., 0.001 cm per sec to 50 cm per sec). The device, lead and/or electrode can then be removed from the solution. The rate at which the device, lead or electrode is withdrawn from the solution can be altered (e.g., 0.001 cm per sec to 50 cm per sec). The coated device, lead or electrode can be air-dried. The dipping process can be repeated one or more times depending on the specific application. The device, lead or electrode can be dried under vacuum to reduce residual solvent levels. This process results in the fibrosis-inhibiting (or gliosis-inhibiting) drug combination, or individual component(s) thereof, being adsorbed into the CRM or neurostimulation device, lead or electrode. The fibrosis-inhibiting (or gliosis-inhibiting) drug combination, or individual component(s) thereof, may also be present on the surface of the device, lead and/or electrode. The amount of surface associated fibrosis-inhibiting (or gliosis-inhibiting) drug combination, or individual component(s) thereof, may be reduced by dipping the coated device, lead or electrode into a solvent for the fibrosis-inhibiting (or gliosis-inhibiting) drug combination, or individual component(s) thereof, or by spraying the coated device, lead or electrode with a solvent for the fibrosis-inhibiting (or gliosis-inhibiting) drug combination, or individual component(s) thereof.

Fibrosis-Inhibiting (or Gliosis-Inhibiting) Drug Combination or Individual Component(s) Thereof with a Solvent

In one embodiment, the solvent is one that may be absorbed by the device, lead or electrode and that will dissolve the device, lead or electrode. The device, lead or electrode can be immersed, either partially or completely, in the fibrosis-inhibiting (or gliosis-inhibiting) drug combination (or individual component(s) thereof)/solvent solution for a specific period of time (seconds to hours). The rate of immersion into the fibrosis-inhibiting (or gliosis-inhibiting) drug combination (or individual component(s) thereof)/solvent solution can be altered (e.g., 0.001 cm per sec to 50 cm per sec). The device, lead or electrode can then be removed from the solution. The rate at which the device, lead or electrode is withdrawn from the solution can be altered (e.g., 0.001 cm per sec to 50 cm per sec). The coated device, lead or electrode can be air-dried. The dipping process can be repeated one or more times depending on the specific application. The device, lead or electrode can be dried under vacuum to reduce residual solvent levels. This process will result in the fibrosis-inhibiting (or gliosis-inhibiting) drug combination, or individual component(s) thereof, being adsorbed into the medical device, lead or electrode as well as being surface associated. The exposure time of the device, lead or electrode to the solvent should not incur significant permanent dimensional changes to the device, lead or electrode. The fibrosis-inhibiting (or gliosis-inhibiting) drug combination, or individual component(s) thereof, may also be present on the surface of the device, lead or electrode. The amount of surface associated fibrosis-inhibiting (or gliosis-inhibiting) drug combination, or individual component(s) thereof, may be reduced by dipping the coated device, lead or electrode into a solvent for the fibrosis-inhibiting (or gliosis-inhibiting) drug combination, or individual component(s) thereof, or by spraying the coated device, lead or electrode with a solvent for the fibrosis-inhibiting (or gliosis-inhibiting) drug combination, or individual component(s) thereof.

In the above description the device, lead or electrode can be one that has not been modified or one that has been further modified by coating with a polymer, surface treated by plasma treatment, flame treatment, corona treatment, surface oxidation or reduction, surface etching, mechanical smoothing or roughening, or grafting prior to the coating process.

In any one the above dip coating methods, the surface of the device, lead or electrode can be treated with a plasma polymerization method prior to coating with the fibrosis-inhibiting (or gliosis-inhibiting) drug combination, or individual component(s) thereof, or with the composition containing the fibrosis-inhibiting (or gliosis-inhibiting) drug combination, or individual component(s) thereof, such that a thin polymeric layer is deposited onto the device, lead or electrode surface. Examples of such methods include parylene coating of devices and the use of various monomers such hydrocyclosiloxane monomers. Parylene coating may be especially advantageous if the device, or portions of the device (such as the lead or the electrode), are composed of materials (e.g., stainless steel, nitinol) that do not allow incorporation of the therapeutic agent(s) into the surface layer using one of the above methods. A parylene primer layer may be deposited onto the electrical device, lead or electrode using a parylene coater (e.g., PDS 2010 LABCOATER2 from Cookson Electronics) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of PARYLENE N, and Parylene D, a dichlorinated derivative of PARYLENE N).

In certain embodiments, the fibrosis-inhibiting (or gliosis-inhibiting) drug combination, or individual component(s) thereof, and a polymer are dissolved in a solvent, for both the polymer and the-fibrosis-inhibiting (or gliosis-inhibiting) drug combination, or individual component(s) thereof, and are then coated onto the device, lead or electrode.

Fibrosis-Inhibiting (or Gliosis-Inhibiting) Drug Combination (or Individual Component(S) Thereof)/Polymer with an Inert-Solvent

In one embodiment, the solvent is an inert solvent for the medical device, lead or electrode, such that the solvent does not dissolve the device, lead or electrode to any great extent and is not absorbed by the device, lead or electrode to any great extent. The device, lead or electrode can be immersed, either partially or completely, in the fibrosis-inhibiting (or gliosis-inhibiting) drug combination (or individual component(s) thereof)/polymer/solvent solution for a specific period of time. The rate of immersion into the fibrosis-inhibiting (or gliosis-inhibiting) drug combination (or individual component(s) thereof)/polymer/solvent solution can be altered (e.g., 0.001 cm per sec to 50 cm per sec). The device, lead or electrode can then be removed from the solution. The rate at which the device, lead or electrode can be withdrawn from the solution can be altered (e.g., 0.001 cm per sec to 50 cm per sec). The coated device, lead or electrode can be air-dried. The dipping process can be repeated one or more times depending on the specific application. The device, lead or electrode can be dried under vacuum to reduce residual solvent levels. This process will result in the fibrosis-inhibiting (or gliosis-inhibiting) drug combination (or individual component(s) thereof)/polymer being coated on the surface of the device, lead or electrode.

Fibrosis-Inhibiting (or Gliosis-Inhibiting) Drug Combination (or Individual Component(S) Thereof)/Polymer with a Swelling Solvent

In one embodiment, the solvent is one that will not dissolve the device, lead or electrode, but will be absorbed by the device, lead or electrode. These solvents can thus swell the device, lead or electrode to some extent. The device, lead or electrode can be immersed, either partially or completely, in the fibrosis-inhibiting (or gliosis-inhibiting) drug combination (or individual component(s) thereof)/polymer/solvent solution for a specific period of time (seconds to days). The rate of immersion into the fibrosis-inhibiting (or gliosis-inhibiting) drug combination (or individual component(s) thereof)/polymer/solvent solution can be altered (e.g., 0.001 cm per sec to 50 cm per sec). The device, lead or electrode can then be removed from the solution. The rate at which the device, lead or electrode can be withdrawn from the solution can be altered (e.g., 0.001 cm per sec to 50 cm per sec). The coated device, lead or electrode can be air-dried. The dipping process can be repeated one or more times depending on the specific application. The device, lead or electrode can be dried under vacuum to reduce residual solvent levels. This process will result in the fibrosis-inhibiting (or gliosis-inhibiting) drug combination (or individual component(s) thereof)/polymer being coated onto the surface of the device, lead or electrode, as well as the potential for the fibrosis-inhibiting (or gliosis-inhibiting) drug combination, or individual component(s) thereof, being adsorbed into the device, lead or electrode. The fibrosis-inhibiting (or gliosis-inhibiting) drug combination, or individual component(s) thereof, may also be present on the surface of the medical device, lead or electrode. The amount of surface associated fibrosis-inhibiting (or gliosis-inhibiting) drug combination, or individual component(s) thereof, may be reduced by dipping the coated device, lead or electrode into a solvent for the fibrosis-inhibiting (or gliosis-inhibiting) drug combination, or individual component(s) thereof, or by spraying the coated device, lead or electrode with a solvent for the fibrosis-inhibiting (or gliosis-inhibiting) drug combination, or individual component(s) thereof.

Fibrosis-Inhibiting (or Gliosis-Inhibiting) Drug Combination (or Individual Component(S) Thereof)/Polymer with a Solvent

In one embodiment, the solvent is one that will be absorbed by the device, lead or electrode and that will dissolve the device, lead or electrode. The device, lead or electrode can be immersed, either partially or completely, in the fibrosis-inhibiting (or gliosis-inhibiting) drug combination (or individual component(s) thereof)/polymer/solvent solution for a specific period of time (seconds to hours). The rate of immersion into the fibrosis-inhibiting (or gliosis-inhibiting) drug combination (or individual component(s) thereof)/polymer/solvent solution can be altered (e.g., 0.001 cm per sec to 50 cm per sec). The device, lead or electrode can then be removed from the solution. The rate at which the device, lead or electrode can be withdrawn from the solution can be altered (e.g., 0.001 cm per sec to 50 cm per sec). The coated device, lead or electrode can be air-dried. The dipping process can be repeated one or more times depending on the specific application. The device, lead or electrode can be dried under vacuum to reduce residual solvent levels. The process will result in the fibrosis-inhibiting (or gliosis-inhibiting) drug combination, or individual component(s) thereof, being adsorbed into the device, lead or electrode, as well as being surface associated. Preferably, the exposure time of the device, lead or electrode to the solvent can be such that there are not significant permanent dimensional changes to the device, lead or electrode (other than those associated with the coating itself). The fibrosis-inhibiting (or gliosis-inhibiting) drug combination, or individual component(s) thereof, may also be present on the surface of the device, lead or electrode. The amount of surface associated fibrosis-inhibiting (or gliosis-inhibiting) drug combination, or individual component(s) thereof, may be reduced by dipping the coated device, lead or electrode into a solvent for the fibrosis-inhibiting (or gliosis-inhibiting) drug combination, or individual component(s) thereof, or by spraying the coated device, lead or electrode with a solvent for the fibrosis-inhibiting drug combination, or individual component(s) thereof.

In the above description, the device, lead or electrode can be one that has not been modified or one that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting drug combination, or individual component(s) thereof, in a polymer solution can be prepared. The suspension can be prepared by choosing a solvent that can dissolve the polymer but not the fibrosis-inhibiting drug combination, or individual component(s) thereof, or a solvent that can dissolve the polymer and in which the fibrosis-inhibiting agent is above its solubility limit. In processes similar to those described above, a device, lead or electrode can be dipped into the suspension of the fibrosis-inhibiting drug combination, or individual component(s) thereof, in the polymer solution such that the device, lead or electrode is coated with a polymer that has a fibrosis-inhibiting drug combination, or individual component(s) thereof, suspended within it.

In any one of the above dip coating methods, the surface of the device, lead or electrode can be treated with a plasma polymerization method prior to coating of a fibrosis-inhibiting (or gliosis-inhibiting) drug combination, or individual component(s) thereof, or a composition that comprises a fibrosis-inhibiting (or gliosis-inhibiting) drug combination, or individual component(s) thereof, such that a thin polymeric layer is deposited onto the device, lead or electrode. Examples of such methods include parylene coating of devices and the use of various monomers such hydrocyclosiloxane monomers. Parylene coating may be especially advantageous if the device, lead, or electrode is composed of materials (e.g., stainless steel, nitinol) that do not allow incorporation of the therapeutic agent(s) into the surface layer using one of the above methods. A parylene primer layer may be deposited onto the device, lead or electrode using a parylene coater (e.g., PDS 2010 LABCOTER2 from Cookson Electronics) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of Parylene N, and PARYLENE D, a dichlorinated derivative of PARYLENE N).

b) Spray Coating CRM and Neurostimulation Devices Leads and Electrodes

Spray coating is another coating process that can be used. In the spray coating process, a solution or suspension of the fibrosis-inhibiting (or gliosis-inhibiting) drug combination, or individual component(s) thereof, with or without a polymeric or non-polymeric carrier, is nebulized and directed to the device, lead and/or electrode to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part #14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the fibrosis-inhibiting (or gliosis-inhibiting) drug combination, or individual component(s) thereof, is dissolved in a solvent for the anti-fibrosis (or anti-gliosis) drug combination, or individual component(s) thereof, and is then sprayed onto the device, lead and/or electrode.

Fibrosis-Inhibiting (or Gliosis-Inhibiting) Drug Combination or Individual Component(s) Thereof, with an Inert Solvent

In one embodiment, the solvent is an inert solvent for the device, lead or electrode such that the solvent does not dissolve the medical device, lead or electrode to any great extent and is not absorbed to any great extent. The device, lead or electrode can be held in place or mounted onto a mandrel or rod that has the ability to move in an X, Y or Z plane or a combination of these planes. Using one of the above described spray devices, the device, lead or electrode can be spray coated such that it is either partially or completely coated with the fibrosis-inhibiting (or gliosis-inhibiting) drug combination (or individual component(s) thereof)/solvent solution. The rate of spraying of the fibrosis-inhibiting (or gliosis-inhibiting) drug combination (or individual component(s) thereof)/solvent solution can be altered (e.g., 0.001 mL per sec to 10 mL per sec) to ensure that a good coating of the fibrosis-inhibiting (or gliosis-inhibiting) drug combination, or individual component(s) thereof, is obtained. The coated device, lead or electrode can be air-dried. The spray coating process can be repeated one or more times depending on the specific application. The device, lead or electrode can be dried under vacuum to reduce residual solvent levels. This process results in the fibrosis-inhibiting (or gliosis-inhibiting) drug combination, or individual component(s) thereof, being coated on the surface of the device, lead and/or electrode.

Fibrosis-Inhibiting (or Gliosis-Inhibiting) Drug Combination, or Individual Component(s) Thereof, with a Swelling Solvent

In one embodiment, the solvent is one that will not dissolve the device, lead or electrode but will be absorbed by it. These solvents can thus swell the device, lead or electrode to some extent. The device, lead or electrode can be spray coated, either partially or completely, in the fibrosis-inhibiting (or gliosis-inhibiting) drug combination (or individual component(s) thereof)/solvent solution. The rate of spraying of the fibrosis-inhibiting (or gliosis-inhibiting) drug combination (or individual component(s) thereof)/solvent solution can be altered (e.g., 0.001 mL per sec to 10 mL per sec) to ensure that a good coating of the fibrosis-inhibiting (or gliosis-inhibiting) drug combination, or individual component(s) thereof, is obtained. The coated device, lead or electrode can be air-dried. The spray coating process can be repeated one or more times depending on the specific application. The device, lead or electrode can be dried under vacuum to reduce residual solvent levels. This process can result in the fibrosis-inhibiting (or gliosis-inhibiting) drug combination, or individual component(s) thereof, being adsorbed into the medical device, lead or electrode. The fibrosis-inhibiting (or gliosis-inhibiting) drug combination, or individual component(s) thereof, may also be present on the surface of the device, lead or electrode. The amount of surface associated fibrosis-inhibiting (or gliosis-inhibiting) drug combination, or individual component(s) thereof, may be reduced by dipping the coated device, lead or electrode into a solvent for the fibrosis-inhibiting (or gliosis-inhibiting) drug combination, individual component(s) thereof, or by spraying the coated device, lead or electrode with a solvent for the fibrosis-inhibiting (or gliosis-inhibiting) drug combination, or individual component(s) thereof.

Fibrosis-Inhibiting (or Gliosis-Inhibiting) Drug Combination or Individual Components Thereof with a Solvent

In one embodiment, the solvent is one that will be absorbed by the device, lead or electrode and that will dissolve it. The device, lead or electrode can be spray coated, either partially or completely, in the fibrosis-inhibiting (or gliosis-inhibiting) drug combination (or individual component(s) thereof)/solvent solution. The rate of spraying of the fibrosis-inhibiting (or gliosis-inhibiting) drug combination (or individual component(s) thereof)/solvent solution can be altered (e.g., 0.001 mL per sec to 10 mL per sec) to ensure that a good coating of the fibrosis-inhibiting (or gliosis-inhibiting) drug combination, or individual component(s) thereof, is obtained. The coated device, lead or electrode can be air-dried. The spray coating process can be repeated one or more times depending on the specific application. The device, lead or electrode can be dried under vacuum to reduce residual solvent levels. This process will result in the fibrosis-inhibiting (or gliosis-inhibiting) drug combination, or individual component(s) thereof, being adsorbed into the medical device, lead or electrode as well as being surface associated. In one embodiment, the exposure time of the device, lead or electrode to the solvent may not incur significant permanent dimensional changes to it. The fibrosis-inhibiting (or gliosis-inhibiting) drug combination, or individual component(s) thereof, may also be present on the surface of the device, lead or electrode. The amount of surface-associated fibrosis-inhibiting (or gliosis-inhibiting) drug combination, or individual component(s) thereof, may be reduced by dipping the coated device, lead or electrode into a solvent for the fibrosis-inhibiting (or gliosis-inhibiting) drug combination, or individual component(s) thereof, or by spraying the coated device, lead or electrode with a solvent for the fibrosis-inhibiting (or gliosis-inhibiting) drug combination, or individual component(s) thereof.

In the above description, the device, lead or electrode can be one that has not been modified as well as one that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In one embodiment, the fibrosis-inhibiting (or gliosis-inhibiting) drug combination, or individual component(s) thereof, and a polymer are dissolved in a solvent for both the polymer and the fibrosis-inhibiting (or gliosis-inhibiting) drug combination, or individual component(s) thereof, and are then spray coated onto the device, lead or electrode.

Fibrosis-Inhibiting (or Gliosis-Inhibiting) Drug Combination (or Individual Component(s) Thereof)/Polymer with an Inert Solvent

In one embodiment, the solvent is an inert solvent for the device, lead or electrode such that the solvent does not dissolve the device, lead or electrode to any great extent and is not absorbed by the device, lead or electrode to any great extent. The device, lead or electrode can be spray coated, either partially or completely, with the fibrosis-inhibiting (or gliosis-inhibiting) drug combination (or individual component(s) thereof)/polymer/solvent solution for a specific period of time. The rate of spraying of the fibrosis-inhibiting (or gliosis-inhibiting) drug combination (or individual component(s) thereof)/polymer/solvent solution can be altered (e.g., 0.001 mL per sec to 10 mL per sec) to ensure that a good coating of the fibrosis-inhibiting (or gliosis-inhibiting) drug combination (or individual component(s) thereof)/polymer is obtained. The coated device, lead or electrode can be air-dried. The spray coating process can be repeated one or more times depending on the specific application. The device, lead or electrode can be dried under vacuum to reduce residual solvent levels. This process can result in the fibrosis-inhibiting (or gliosis-inhibiting) drug combination (or individual component(s) thereof)/polymer being coated on the surface of the device, lead or electrode.

Fibrosis-Inhibiting (or Gliosis-Inhibiting) Drug Combination (or Individual Component(s) Thereof)/Polymer with a Swelling Solvent

In one embodiment, the solvent is one that will not dissolve the device, lead or electrode but will be absorbed by it. These solvents can thus swell the device, lead or electrode to some extent. The device, lead or electrode can be spray coated, either partially or completely, with the fibrosis-inhibiting (or gliosis-inhibiting) drug combination (or individual component(s) thereof)/polymer/solvent solution. The rate of spraying of the fibrosis-inhibiting (or gliosis-inhibiting) drug combination (or individual component(s) thereof)/polymer/solvent solution can be altered (e.g., 0.001 mL per sec to 10 mL per sec) to ensure that a good coating of the fibrosis-inhibiting (or gliosis-inhibiting) drug combination (or individual component(s) thereof)/polymer is obtained. The coated device, lead or electrode can be air-dried. The spray coating process can be repeated one or more times depending on the specific application. The device, lead or electrode can be dried under vacuum to reduce residual solvent levels. This process will result in the fibrosis-inhibiting (or gliosis-inhibiting) drug combination (or individual component(s) thereof)/polymer being coated onto the surface of the device, lead or electrode, as well as the potential for the fibrosis-inhibiting (or gliosis-inhibiting) drug combination, or individual component(s) thereof, being adsorbed into the medical device, lead or electrode. The fibrosis-inhibiting (or gliosis-inhibiting) drug combination, or individual component(s) thereof, may also be present on the surface of the device, lead or electrode. The amount of surface associated fibrosis-inhibiting (or gliosis-inhibiting) drug combination, or individual component(s) thereof, may be reduced by dipping the coated device, lead or electrode into a solvent for the fibrosis-inhibiting (or gliosis-inhibiting) drug combination, or individual component(s) thereof, or by spraying the coated device, lead or electrode with a solvent for the fibrosis-inhibiting (or gliosis-inhibiting) drug combination, or individual component(s) thereof.

Fibrosis-Inhibiting (or Gliosis-Inhibiting) Drug Combination (or Individual Components Thereof)/Polymer with a Solvent

In one embodiment, the solvent is one that will be absorbed by the device, lead or electrode and that will dissolve it. The device, lead or electrode can be spray coated, either partially or completely, with the fibrosis-inhibiting (or gliosis-inhibiting) drug combination (or individual component(s) thereof)/polymer/solvent solution. The rate of spraying of the fibrosis-inhibiting (or gliosis-inhibiting) drug combination (or individual component(s) thereof)/polymer/solvent solution can be altered (e.g., 0.001 mL per sec to 10 mL per sec) to ensure that a good coating of the fibrosis-inhibiting (or gliosis-inhibiting) drug combination (or individual component(s) thereof)/polymer is obtained. The coated device, lead or electrode can be air-dried. The spray coating process can be repeated one or more times depending on the specific application. The device, lead or electrode can be dried under vacuum to reduce residual solvent levels. In the preferred embodiment, the exposure time of the device, lead or electrode to the solvent may not incur significant permanent dimensional changes to it (other than those associated with the coating itself). The fibrosis-inhibiting (or gliosis-inhibiting) drug combination, or individual component(s) thereof, may also be present on the surface of the device, lead or electrode. The amount of surface associated fibrosis-inhibiting (or gliosis-inhibiting) drug combination, or individual component(s) thereof, may be reduced by dipping the coated device, lead or electrode into a solvent for the fibrosis-inhibiting (or gliosis-inhibiting) drug combination, or individual component(s) thereof, or by spraying the coated device, lead or electrode with a solvent for the fibrosis-inhibiting (or gliosis-inhibiting) drug combination, or individual component(s) thereof.

In the above description the device, lead or electrode can be one that has not been modified as well as one that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting (or gliosis-inhibiting) drug combination, or individual component(s) thereof, in a polymer solution can be prepared. The suspension can be prepared by choosing a solvent that can dissolve the polymer but not the fibrosis-inhibiting (or gliosis-inhibiting) drug combination, or individual component(s) thereof, or a solvent that can dissolve the polymer and in which the fibrosis-inhibiting (or gliosis-inhibiting) drug combination, or individual component(s) thereof, is above its solubility limit. In processes similar to those described above, the suspension of the fibrosis-inhibiting (or gliosis-inhibiting) drug combination, or individual component(s) thereof, in the polymer solution can be sprayed onto the CRM or neurostimulation device, lead or electrode such that it is coated with a polymer that has a fibrosis-inhibiting (or gliosis-inhibiting) drug combination, or individual component(s) thereof, suspended within it.

c) Sequential Coating Process

In other embodiments, one of the drugs of the combination can be applied as described in the dip and/or spray coating methods above and then this can be followed by a second coating process, using one of the methods described above, in which the second drug of the combination is coated onto the device.

d) Top Coat Process

In another embodiment, once any of the dip coating or spray coating processes described above have been completed, the drug-loaded device can be coated with a top coat of a polymer solution. This top coat can provide a means to modulate the release profiles of the drugs. The top coat can comprise the same polymer as the drug-containing coating polymer or the can be of a different molecular weight and/or a different composition than the drug-containing coating.

In another embodiment, the top coat layer can further comprise a biologically active agent. Examples of these agents can include anti-thrombotic agents, anti-platelet agents, anti-inflammatory agents or anti-bacterial agents.

In another embodiment, the top coat can alter the surface properties of the device. For example, the top coat can provide lubricity to the surface and/or the top coat can either enhance or decrease the surface smoothness and/or porosity.

e) Drug Combination Ratios

In another embodiment, the ratio of each drug in the drug combination composition that is used to drug load the device can be altered. For example, if one has a drug combination comprising drug A and drug B, then the ratio of A:B can be altered when preparing the reagents for the processes (as described above) for drug loading the devices. For illustrative purposes, one could have a ratio of A:B of 10:90, 20:80, 30:70, 40:60, 50:50, 60:40, 70:30, 80:20 and 90:10 as well an other intermediate ratios not specifically listed.

2) Systemic, Regional and Local Delivery of Fibrosis-Inhibiting (or Gliosis-Inhibiting) Drug Combinations of Individual Component(s) Thereof

A variety of drug-delivery technologies are available for systemic, regional and local delivery of therapeutic agents. Several of these techniques may be suitable to achieve preferentially elevated levels of fibrosis-inhibiting (or gliosis-inhibiting) drug combinations, or individual component(s) thereof, in the vicinity of the CRM or neurostimulation device, lead and/or electrode, including: (a) using drug-delivery catheters for local, regional or systemic delivery of fibrosis-inhibiting (or gliosis-inhibiting) drug combinations, or individual component(s) thereof, to the tissue surrounding the device or implant. Typically, drug delivery catheters are advanced through the circulation or inserted directly into tissues under radiological guidance until they reach the desired anatomical location. The fibrosis-inhibiting drug combination, or individual component(s) thereof, can then be released from the catheter lumen in high local concentrations in order to deliver therapeutic doses of the drug to the tissue surrounding the device or implant; (b) drug localization techniques such as magnetic, ultrasonic or MRI-guided drug delivery; (c) chemical modification of the fibrosis-inhibiting (or gliosis-inhibiting) drug(s) or formulation designed to increase uptake of the drug combinations, or individual component(s) thereof, into damaged tissues (e.g., antibodies directed against damaged or healing tissue components such as macrophages, neutrophils, smooth muscle cells, fibroblasts, extracellular matrix components, neovascular tissue); (d) chemical modification of the fibrosis-inhibiting (or gliosis-inhibiting) drug(s) or formulation designed to localize the drug combination, or individual component(s) thereof, to areas of bleeding or disrupted vasculature; and/or (e) direct injection of the fibrosis-inhibiting (or gliosis-inhibiting) drug combination, or individual component(s) thereof, for example, under endoscopic vision.

3) Infiltration of Fibrosis-Inhibiting (or Gliosis-Inhibiting) Drug Combinations, or Individual Component(s) Thereof, into the Tissue Surrounding a Device or Implant

Alternatively, the tissue surrounding the CRM or neurostimulation device can be treated with a fibrosis-inhibiting (or gliosis-inhibiting) drug combination, or individual component(s) thereof, or a composition that comprises a fibrosis-inhibiting (or gliosis-inhibiting) drug combination, or individual component(s) thereof, prior to, during, or after the implantation procedure. A fibrosis-inhibiting (or gliosis-inhibiting) drug combination, or individual component(s) thereof, or a composition comprising a fibrosis-inhibiting (or gliosis-inhibiting) drug combination, or individual component(s) thereof, may be infiltrated around the device or implant by applying the composition directly and/or indirectly into and/or onto (a) tissue adjacent to the medical device; (b) the vicinity of the medical device-tissue interface; (c) the region around the medical device; and (d) tissue surrounding the medical device.

Methods for infiltrating the subject compositions into tissue adjacent to a medical device include delivering the composition of fibrosis-inhibiting (or gliosis-inhibiting) drug combination, or individual component(s) thereof: (a) to the medical device surface (e.g., as an injectable, paste, gel or mesh) during the implantation procedure; (b) to the surface of the tissue (e.g., as an injectable, paste, gel, in situ forming gel or mesh) immediately prior to, or during, implantation of the medical device; (c) to the surface of the medical device and/or the tissue surrounding the implanted medical device (e.g., as an injectable, paste, gel, in situ forming gel or mesh) immediately after the implantation of the medical device; (d) by topical application of the composition into the anatomical space where the medical device may be placed (particularly useful for this embodiment is the use of polymeric carriers which release the therapeutic drug combination, or individual component(s) thereof, over a period ranging from several hours to several weeks—fluids, suspensions, emulsions, microemulsions, microspheres, pastes, gels, microparticulates, sprays, aerosols, solid implants and other formulations which release the drug combination, or individual component(s) thereof, may be delivered into the region where the device may be inserted); (e) via percutaneous injection into the tissue surrounding the medical device as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

It should be noted that certain polymeric carriers themselves can help prevent the formation of fibrous or gliotic tissue around the CRM or neuroimplant. These carriers are particularly useful for the practice of this embodiment, either alone, or in combination with a fibrosis-inhibiting (or gliosis-inhibiting) composition. The following polymeric carriers can be infiltrated (as described in the previous paragraph) into the vicinity of the electrode-tissue interface and include: (a) sprayable collagen-containing formulations such as COSTASIS and CT3, either alone, or loaded with a fibrosis-inhibiting (or gliosis-inhibiting) drug combination, or individual component(s) thereof, applied to the implantation site (or the implant/device surface); (b) sprayable PEG-containing formulations such as COSEAL, FOCALSEAL, SPRAYGEL or DURASEAL, either alone, or loaded with a fibrosis-inhibiting (or gliosis-inhibiting) drug combination, or individual component(s) thereof, applied to the implantation site (or the implant/device surface); (c) fibrinogen-containing formulations such as FLOSEAL or TISSEAL, either alone, or loaded with a fibrosis-inhibiting (or gliosis-inhibiting) drug combination, or individual component(s) thereof, applied to the implantation site (or the implant/device surface); (d) hyaluronic acid-containing formulations such as RESTYLANE, HYLAFORM, PERLANE, SYNVISC, SEPRAFILM, SEPRACOAT, InterGel, LUBRICOAT, loaded with a fibrosis-inhibiting (or gliosis-inhibiting) drug combination, or individual component(s) thereof, applied to the implantation site (or the implant/device surface); (e) polymeric gels for surgical implantation such as REPEL or FLOWGEL loaded with a fibrosis-inhibiting (or gliosis-inhibiting) drug combination, or individual component(s) thereof, applied to the implantation site (or the implant/device surface); (f) orthopedic “cements” used to hold prostheses and tissues in place loaded with a fibrosis-inhibiting (or gliosis-inhibiting) drug combination, or individual component(s) thereof, applied to the implantation site (or the implant/device surface), such as OSTEOBOND (Zimmer), low viscosity cement (LVC) (Wright Medical Technology), SIMPLEX P (Stryker), PALACOS (Smith & Nephew), and ENDURANCE (Johnson & Johnson, Inc.); (g) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT, either alone, or loaded with a fibrosis-inhibiting (or gliosis-inhibiting) drug combination, or individual component(s) thereof, applied to the implantation site (or the implant/device surface); (h) implants containing hydroxyapatite [or synthetic bone material such as calcium sulfate, VITOSS (Orthovita) and CORTOSS (Orthovita)] loaded with a fibrosis-inhibiting (or gliosis-inhibiting) drug combination, or individual component(s) thereof applied to the implantation site (or the implant/device surface); (i) other biocompatible tissue fillers loaded with a fibrosis-inhibiting (or gliosis-inhibiting) drug combination, or individual component(s) thereof, such as those made by BioCure, 3M Company and Neomend, applied to the implantation site (or the implant/device surface); (j) polysaccharide gels such as the ADCON series of gels either alone, or loaded with a fibrosis-inhibiting (or gliosis-inhibiting) drug combination, or individual component(s) thereof, applied to the implantation site (or the implant/device surface); and/or (k) films, sponges or meshes such as INTERCEED, VICRYL mesh, and GELFOAM loaded with a fibrosis-inhibiting (or gliosis-inhibiting) drug combination, or individual component(s) thereof, applied to the implantation site (or the implant/device surface).

An exemplary polymeric matrix which can be used to help prevent the formation of fibrous or gliotic tissue around the CRM or neuroimplant, either alone or in combination with a fibrosis-inhibiting (or gliosis-inhibiting) drug combination, or individual component(s) thereof, or a composition comprising a drug combination, or individual component(s) thereof, may be formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another exemplary composition may comprise either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous or gliotic tissue around the CRM or neuroimplant.

Other examples of compositions that may be infiltrated into tissue adjacent to a medical device include compositions formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another exemplary composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix.

In certain embodiments, individual components of drug combinations are combined together before being used to locally infiltrate into a tissue. In certain other embodiments, individual components of drug combinations are used to separately infiltrate a tissue and thus form a drug combination in the tissue.

A drug dose administered from the present compositions will depend on a variety of factors, including the type of formulation, the location of the treatment site, and the type of condition being treated, as well as the surface area of the device. However, certain principles can be applied in the application of this art. Drug dose can be calculated as a function of dose per unit area (of the treatment site), wherein the total drug dose administered can be measured and appropriate surface concentrations of active drug can be determined. Drugs are to be used at concentrations that range from several times more than to 50%, 20%, 10%, 5%, or even less than 1% of the concentration typically used in a single systemic dose application. In certain aspects, the anti-scarring or anti-gliosis drug combination, or individual component(s) thereof, is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In certain embodiments, the drug is released in effective concentrations for a period ranging from 1-90 days. It should be understood in certain embodiments that within the drug combination, one drug may be released at a different rate and/or for a different amount of time than the other drug(s).

The exemplary anti-fibrosing or anti-gliosis drug combinations or individual components thereof should be administered under the following dosing guidelines. The total amount (dose) of anti-scarring or anti-gliosis agent(s) in the drug combinations or compositions that comprise the drug combinations can be in the range of about 0.01 μg-10 μg, or 10 μg-100 μg, or 100 μg-1000 μg, or 1 mg-10 mg, or 10 mg-250 mg, or 250 mg-1000 mg, or 1000 mg-2500 mg. The dose (amount) of anti-scarring or anti-gliosis agent(s) per unit area of surface to which the agent is applied may be in the range of about 0.01 μg/mm²-1 μg/mm², or 1 μg/mm²-10 μg/mm², or 10 μg/mm²-250 μg/mm², or 250 μg/mm²-1000 μg/mm², or 1000 μg/mm²-2500 μg/mm².

Provided below are exemplary drug combinations and dosage ranges for various anti-scarring and/or anti-gliosis drug combinations or individual components thereof that can be used in conjunction with devices in accordance with the invention.

Exemplary anti-fibrotic drug combinations for description of dosing include, but are not limited to amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, and analogues and derivatives thereof: total dose of each drug within the combination not to exceed 500 mg (range of 0.1 μg to 500 mg; preferred 1 μg to 200 mg). Dose per unit area of 0.01 μg/mm² to 200 μg/mm²; preferred dose of 0.1 μg/mm² to 100 μg/mm². Minimum concentration of 10⁻⁸ to 10⁻⁴M of agent is to be maintained on the implant or barrier surface. Molar ratio of each drug in the combination is to be within the range of 1:1 to 1:1000. Molar ratios within this range may include but are not limited to 1:5, 1:10, 1:15, 1:20, 1:30, 1:50, 1:75, 1:100, 1:200, 1:500, 1:1000. Note that molar ratios may also lie between the ratios stated above.

Neurostimulation Devices

In one aspect, an anti-fibrosis (or anti-gliosis) drug combination, or individual component(s) thereof, or a composition that comprises an anti-fibrosis (or anti-gliosis) drug combination, or individual component(s) thereof) may be infiltrated into tissue adjacent to an implantable neurostimulation device, lead or electrode.

Any implantable device may benefit from having an fibrosis-inhibiting (or gliosis-inhibiting) drug combination, or individual component(s) thereof, or a composition that comprises an fibrosis-inhibiting (or gliosis-inhibiting) drug combination, or individual component(s) thereof, infiltrated into adjacent tissue according to the present invention.

Agents or compositions of the present invention may be infiltrated around implantable devices, leads or electrodes by applying the composition directly and/or indirectly into and/or onto (a) tissue adjacent to the device, lead or electrode; (b) the vicinity of the interface between the tissue and the device, lead or electrode; (c) the region around the device, lead or electrode; and (d) tissue surrounding the device, lead or electrode. Methods for infiltrating an fibrosis-inhibiting (or gliosis-inhibiting) drug combination, or individual component(s) thereof, into tissue adjacent to a device, lead or electrode include delivering the drug combination, or individual component(s) thereof, or a composition comprising drug combination, or individual component(s) thereof: (a) to the surface of the device, lead or electrode (e.g., as an injectable, paste, gel or mesh) during the implantation procedure; (b) to the surface of the tissue (e.g., as an injectable, paste, gel, in situ forming gel or mesh) immediately prior to, or during, implantation of the device, lead or electrode; (c) to the surface of the device, lead or electrode and/or the tissue surrounding the device, lead or electrode (e.g., as an injectable, paste, gel, in situ forming gel or mesh) immediately after the implantation of the device, lead or electrode; (d) by topical application of the drug combination, or individual component(s) thereof, or a composition comprising the drug combination, or individual component(s) thereof, into the anatomical space where the device, lead or electrode may be placed (particularly useful for this embodiment is the use of polymeric carriers which release the drug combination, or individual component(s) thereof, over a period ranging from several hours to several weeks—fluids, suspensions, emulsions, microemulsions, microspheres, pastes, gels, microparticulates, sprays, aerosols, solid implants and other formulations which release the drug combination, or individual component(s) thereof, may be delivered into the region where the device may be inserted); (e) via percutaneous injection into the tissue surrounding the device, lead or electrode as a solution, as an infusate, or as a sustained-release preparation; (f) by any combination of the aforementioned methods. Combination therapies (e.g., combinations with antithrombotic and/or antiplatelet agents) may also be used. In all cases it is understood that the subject compositions may be infiltrated into tissue adjacent to all or a portion of the device, lead or electrode.

According to one aspect, any fibrosis-inhibiting (or gliosis-inhibiting) drug combination, or individual component(s) thereof, or a composition comprising a fibrosis-inhibiting (or gliosis-inhibiting) drug combination, or individual component(s) thereof, or a composition described herein may be utilized in the practice of the present invention. In one aspect of the invention, the subject compositions infiltrated into tissue adjacent to neurostimulation devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the present compositions for prevention or inhibition of fibrosis (or gliosis) in accordance with the present invention will depend on a variety of factors, including the type of formulation, the location of the treatment site, and the type of condition being treated. As neurostimulation devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring drug combination, or individual component(s) thereof, is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the drug combinations or compositions that comprise the drug combinations can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

For greater clarity, several specific neurostimulation devices and treatments will be described in greater detail below.

(1) Neurostimulation for the Treatment of Chronic Pain

Neurostimulation devices for the treatment of chronic pain may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such neurostimulation devices for the treatment of chronic pain are provided above, together with methods for coating such neurostimulation devices. Numerous polymeric and non-polymeric delivery systems for use in connection with neurostimulation devices for the treatment of chronic pain have been described above.

In one aspect, an anti-scarring drug combination, or individual component(s) thereof, or a composition comprising an anti-scarring drug combination, or individual component(s) thereof, may be infiltrated around implanted neurostimulation devices for the management of chronic pain by applying the composition directly and/or indirectly into and/or onto (a) tissue adjacent to the neurostimulation device for the management of chronic pain; (b) the vicinity of the neurostimulation device for the management of chronic pain-tissue interface; (c) the region around the neurostimulation device for the management of chronic pain; and (d) tissue surrounding the neurostimulation device for the management of chronic pain. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a neurostimulation device for the management of chronic pain include delivering the composition: (a) to the surface of the neurostimulation device for the management of chronic pain (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the management of chronic pain; (c) to the surface of the neurostimulation device for the management of chronic pain and/or the tissue surrounding the implanted neurostimulation device for the management of chronic pain (e.g., as an injectable, paste, gel, in situ forming gel or mesh) immediately after the implantation of the neurostimulation device for the management of chronic pain; (d) by topical application of the composition into the anatomical space where the neurostimulation device for the management of chronic pain may be placed (particularly useful for this embodiment is the use of polymeric carriers which release the therapeutic drug combination, or individual component(s) thereof, over a period ranging from several hours to several weeks—fluids, suspensions, emulsions, microemulsions, microspheres, pastes, gels, microparticulates, sprays, aerosols, solid implants and other formulations which release the drug combination, or individual component(s) thereof, may be delivered into the region where the device may be inserted); (e) via percutaneous injection into the tissue surrounding the neurostimulation device for the management of chronic pain as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to neurostimulation devices for the management of chronic pain may be adapted to release a drug combination, or individual component(s) thereof, that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As neurostimulation devices for the management of chronic pain are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-fibrosis (or anti-gliosis) drug combination, or individual component(s) thereof, is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the drug combinations or compositions that comprise the drug combinations can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 g/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(2) Neurostimulation for the Treatment of Parkinson's Disease

Neurostimulation devices for the treatment of Parkinson's Disease may benefit from having an anti-scarring drug combination, or individual component(s) thereof, or a composition comprising and anti-scarring drug composition, or individual component(s) thereof, infiltrated into adjacent tissue according to the present invention. Representative examples of such neurostimulation devices for the treatment of Parkinson's Disease are provided above, together with methods for coating such neurostimulation devices.

In one aspect, an anti-scarring drug combination, or individual component(s) thereof, or a composition comprising an anti-scarring drug combination, or individual component(s) thereof, may be infiltrated into tissue adjacent to where the device and/or leads (e.g., DBS leads) are or will be implanted. In another aspect, the present invention provides DBS leads having the subject composition comprising an anti-scarring drug combination, or individual component(s) thereof, infiltrated into brain tissue adjacent to where the electrodes of the leads are or will be implanted. Numerous polymeric and non-polymeric delivery systems for use in connection with neurostimulation devices for the treatment of Parkinson's disease have been described above.

Anti-fibrosis (or anti-gliosis) drug combinations, or individual component(s) thereof, or pharmaceutical compositions that comprise anti-fibrosis (or anti-gliosis) drug combinations, or individual component(s) thereof, may be infiltrated around implanted neurostimulation devices for the treatment of Parkinson's disease by applying the composition directly and/or indirectly into and/or onto (a) tissue adjacent to the neurostimulation device for the treatment of Parkinson's disease; (b) the vicinity of the neurostimulation device for the treatment of Parkinson's disease-tissue interface; (c) the region around the neurostimulation device for the treatment of Parkinson's disease; and (d) tissue surrounding the neurostimulation device for the treatment of Parkinson's disease. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a neurostimulation device for the treatment of Parkinson's disease include delivering the composition: (a) to the surface of the neurostimulation device for the treatment of Parkinson's disease (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of Parkinson's disease; (c) to the surface of the neurostimulation device for the treatment of Parkinson's disease and/or the tissue surrounding the implanted neurostimulation device for the treatment of Parkinson's disease (e.g., as an injectable, paste, gel, in situ forming gel or mesh) immediately after the implantation of the neurostimulation device for the treatment of Parkinson's disease; (d) by topical application of the composition into the anatomical space where the neurostimulation device for the treatment of Parkinson's disease may be placed (particularly useful for this embodiment is the use of polymeric carriers which release the anti-fibrosis (or anti-gliosis) drug combination, or individual component(s) thereof, over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of Parkinson's disease as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to neurostimulation devices for the treatment of Parkinson's disease may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As neurostimulation devices for the treatment of Parkinson's disease are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-fibrosis (or anti-gliosis) drug combination, or individual component(s) thereof, is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the drug combinations or compositions that comprise the drug combinations can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μmm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(3) Vagal Nerve Stimulation for the Treatment of Epilepsy

Neurostimulation devices for the treatment of epilepsy may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such neurostimulation devices for the treatment of epilepsy are provided above, together with methods for coating such neurostimulation devices. Numerous polymeric and non-polymeric delivery systems for use in connection with neurostimulation devices for the treatment of epilepsy have been described above.

Anti-fibrosis (or anti-gliosis) drug combinations, or individual component(s) thereof, or pharmaceutical compositions that comprise anti-fibrosis (or anti-gliosis) drug combinations, or individual component(s) thereof, may be infiltrated around implanted neurostimulation devices for the treatment of epilepsy by applying the composition directly and/or indirectly into and/or onto (a) tissue adjacent to the neurostimulation device for the treatment of epilepsy; (b) the vicinity of the neurostimulation device for the treatment of epilepsy-tissue interface; (c) the region around the neurostimulation device for the treatment of epilepsy; and (d) tissue surrounding the neurostimulation device for the treatment of epilepsy. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a neurostimulation device for the treatment of epilepsy include delivering the composition: (a) to the surface of the neurostimulation device for the treatment of epilepsy (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of epilepsy; (c) to the surface of the neurostimulation device for the treatment of epilepsy and/or the tissue surrounding the implanted neurostimulation device for the treatment of epilepsy (e.g., as an injectable, paste, gel, in situ forming gel or mesh) immediately after the implantation of the neurostimulation device for the treatment of epilepsy; (d) by topical application of the composition into the anatomical space where the neurostimulation device for the treatment of epilepsy may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of epilepsy as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof described above may be utilized in the practice of the present invention. In one aspect of the invention, the subject compositions infiltrated into tissue adjacent to neurostimulation devices for the treatment of epilepsy may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As neurostimulation devices for the treatment of epilepsy are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-fibrosis (or anti-gliosis) drug combination, or individual component(s) thereof, is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the drug combinations or compositions that comprise the drug combinations can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷ or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(4) Vagal Nerve Stimulation for the Treatment of Other Disorders

Neurostimulation devices for the treatment of neurological disorders may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such neurostimulation devices for the treatment of neurological disorders are provided above, together with methods for coating such neurostimulation devices. Numerous polymeric and non-polymeric delivery systems for use in connection with neurostimulation devices for the treatment of neurological disorders have been described above.

Anti-fibrosis (or anti-gliosis) drug combinations, or individual component(s) thereof, or pharmaceutical compositions that comprise anti-fibrosis (or anti-gliosis) drug combinations, or individual component(s) thereof, may be infiltrated around implanted neurostimulation devices for the treatment of neurological disorders by applying the composition directly and/or indirectly into and/or onto (a) tissue adjacent to the neurostimulation device for the treatment of neurological disorders; (b) the vicinity of the neurostimulation device for the treatment of neurological disorders-tissue interface; (c) the region around the neurostimulation device for the treatment of neurological disorders; and (d) tissue surrounding the neurostimulation device for the treatment of neurological disorders. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a neurostimulation device for the treatment of neurological disorders include delivering the composition: (a) to the surface of the neurostimulation device for the treatment of neurological disorders (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of neurological disorders; (c) to the surface of the neurostimulation device for the treatment of neurological disorders and/or the tissue surrounding the implanted neurostimulation device for the treatment of neurological disorders (e.g., as an injectable, paste, gel, in situ forming gel or mesh) immediately after the implantation of the neurostimulation device for the treatment of neurological disorders; (d) by topical application of the composition into the anatomical space where the neurostimulation device for the treatment of neurological disorders may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of neurological disorders as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to neurostimulation devices for the treatment of neurological disorders may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As neurostimulation devices for the treatment of neurological disorders are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(5) Sacral Nerve Stimulation for Bladder Control Problems

Sacral nerve stimulation devices for the treatment of bladder control problems may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such sacral nerve stimulation devices for the treatment of bladder control problems are provided above, together with methods for coating such devices. Numerous polymeric and non-polymeric delivery systems for use in connection with sacral nerve stimulation devices for the treatment of bladder control problems have been described above.

Anti-fibrosis (or anti-gliosis) drug combinations, or individual component(s) thereof, or pharmaceutical compositions that comprise anti-fibrosis (or anti-gliosis) drug combinations, or individual component(s) thereof, may be infiltrated around implanted neurostimulation systems to treat bladder conditions by applying the composition directly and/or indirectly into and/or onto (a) tissue adjacent to the neurostimulation system to treat bladder conditions; (b) the vicinity of the neurostimulation system to treat bladder conditions-tissue interface; (c) the region around the neurostimulation system to treat bladder conditions; and (d) tissue surrounding the neurostimulation system to treat bladder conditions. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a neurostimulation system to treat bladder conditions include delivering the composition: (a) to the surface of the neurostimulation system to treat bladder conditions (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation system to treat bladder conditions; (c) to the surface of the neurostimulation system to treat bladder conditions and/or the tissue surrounding the implanted neurostimulation system to treat bladder conditions (e.g., as an injectable, paste, gel, in situ forming gel or mesh) immediately after the implantation of the neurostimulation system to treat bladder conditions; (d) by topical application of the composition into the anatomical space where the neurostimulation system to treat bladder conditions may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation system to treat bladder conditions as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to neurostimulation systems to treat bladder conditions may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As neurostimulation systems to treat bladder conditions are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(6) Gastric Nerve Stimulation for the Treatment of Gastrointestinal Disorders

Gastric nerve stimulation devices for the treatment of gastrointestinal (GI) disorders may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such gastric nerve stimulation devices for the treatment of GI disorders are provided above, together with methods for coating such devices. Numerous polymeric and non-polymeric delivery systems for use in connection with gastric nerve stimulation devices for the treatment of GI disorders have been described above.

Anti-fibrosis (or anti-gliosis) drug combinations, or individual component(s) thereof, or pharmaceutical compositions that comprise anti-fibrosis (or anti-gliosis) drug combinations, or individual component(s) thereof, may be infiltrated around implanted devices for treatment of GI disorders by applying the composition directly and/or indirectly into and/or onto (a) tissue adjacent to the device for treatment of GI disorders; (b) the vicinity of the device for treatment of GI disorders-tissue interface; (c) the region around the device for treatment of GI disorders; and (d) tissue surrounding the device for treatment of GI disorders. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a device for treatment of GI disorders include delivering the composition: (a) to the surface of the device for treatment of GI disorders (e.g., as an injectable, paste, gel or mesh) during the implantation procedure; (b) to the surface of the tissue (e.g., as an injectable, paste, gel, in situ forming gel or mesh) immediately prior to, or during, implantation of the device for treatment of GI disorders; (c) to the surface of the device for treatment of GI disorders and/or the tissue surrounding the implanted device for treatment of GI disorders (e.g., as an injectable, paste, gel, in situ forming gel or mesh) immediately after the implantation of the device for treatment of GI disorders; (d) by topical application of the composition into the anatomical space where the device for treatment of GI disorders may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the device for treatment of GI disorders as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to devices for treatment of GI disorders may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As devices for treatment of GI disorders are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(7) Cochlear Implants for the Treatment of Deafness

Cochlear implants for the treatment of deafness may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such cochlear implants for the treatment of deafness are provided above, together with methods for coating such devices. Numerous polymeric and non-polymeric delivery systems for use in connection with cochlear implants for the treatment of deafness have been described above.

Anti-fibrosis (or anti-gliosis) drug combinations, or individual component(s) thereof, or pharmaceutical compositions that comprise anti-fibrosis (or anti-gliosis) drug combinations, or individual component(s) thereof, may be infiltrated around implanted cochlear implants by applying the composition directly and/or indirectly into and/or onto (a) tissue adjacent to the cochlear implant; (b) the vicinity of the cochlear implant-tissue interface; (c) the region around the cochlear implant; and (d) tissue surrounding the cochlear implant. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a cochlear implant include delivering the composition: (a) to the surface of the cochlear implant (e.g., as an injectable, paste, gel or mesh) during the implantation procedure; (b) to the surface of the tissue (e.g., as an injectable, paste, gel, in situ forming gel or mesh) immediately prior to, or during, implantation of the cochlear implant; (c) to the surface of the cochlear implant and/or the tissue surrounding the implanted cochlear implant (e.g., as an injectable, paste, gel, in situ forming gel or mesh) immediately after the implantation of the cochlear implant; (d) by topical application of the composition into the anatomical space where the cochlear implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cochlear implant as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to cochlear implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As cochlear implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 g/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(8) Electrical Stimulation to Promote Bone Growth

Devices for electrical stimulation to promote bone growth may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Numerous polymeric and non-polymeric delivery systems for use in connection with devices for electrical stimulation to promote bone growth have been described above.

Anti-fibrosis (or anti-gliosis) drug combinations, or individual component(s) thereof, or pharmaceutical compositions that comprise anti-fibrosis (or anti-gliosis) drug combinations, or individual component(s) thereof, may be infiltrated around implanted electrical bone stimulation devices by applying the composition directly and/or indirectly into and/or onto (a) tissue adjacent to the electrical bone stimulation device; (b) the vicinity of the electrical bone stimulation device-tissue interface; (c) the region around the electrical bone stimulation device; and (d) tissue surrounding the electrical bone stimulation device. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to an electrical bone stimulation device include delivering the composition: (a) to the surface of the electrical bone stimulation device (e.g., as an injectable, paste, gel or mesh) during the implantation procedure; (b) to the surface of the tissue (e.g., as an injectable, paste, gel, in situ forming gel or mesh) immediately prior to, or during, implantation of the electrical bone stimulation device; (c) to the surface of the electrical bone stimulation device and/or the tissue surrounding the implanted electrical bone stimulation device (e.g., as an injectable, paste, gel, in situ forming gel or mesh) immediately after the implantation of the electrical bone stimulation device; (d) by topical application of the composition into the anatomical space where the electrical bone stimulation device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the electrical bone stimulation device as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to electrical bone stimulation devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As electrical bone stimulation devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-100 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Although numerous neurostimulation devices have been described above, all possess similar design features and cause similar unwanted tissue reactions following implantation and may introduce or promote infection in the area of the implant site. It should be obvious to one of skill in the art that commercial neurostimulation devices not specifically cited above as well as next-generation and/or subsequently developed commercial neurostimulation products are to be anticipated and are suitable for use under the present invention. The neurostimulation device, particularly the lead(s), must be positioned in a very precise manner to ensure that stimulation is delivered to the correct anatomical location in the nervous system. All, or parts, of a neurostimulation device can migrate following surgery, or excessive scar (or glial) tissue growth can occur around the implant, which can lead to a reduction in the performance of these devices. Neurostimulator devices having the subject compositions infiltrated into tissue adjacent to the electrode-tissue interface can be used to increase the efficacy and/or the duration of activity of the implant (particularly for fully-implanted, battery-powered devices). Neurostimulator devices may also benefit from release of a therapeutic agent able to prevent or inhibit infection in the vicinity of the implant site. In one aspect, the present invention provides neurostimulator devices having the subject compositions infiltrated into adjacent tissue, where the subject compositions may include an anti-fibrosis (or anti-gliosis) drug combination, or individual component(s) thereof. Numerous polymeric and non-polymeric delivery systems for use in connection with neurostimulator devices have been described above. These compositions can further include one or more fibrosis-inhibiting agents such that the overgrowth of granulation, fibrous, or gliotic tissue is inhibited or reduced and/or one or more anti-infective agents such that infection in the vicinity of the implant site is inhibited or prevented.

Cardiac Rhythm Management (CRM) Devices

Cardiac rhythm management devices may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such cardiac rhythm management devices are provided above, together with methods for coating such devices. Numerous polymeric and non-polymeric delivery systems for use in connection with cardiac rhythm management devices have been described above.

Anti-fibrosis drug combinations, or individual component(s) thereof, or pharmaceutical compositions that comprise anti-fibrosis drug combinations, or individual component(s) thereof, may be infiltrated around implanted cardiac rhythm management devices by applying the composition directly and/or indirectly into and/or onto (a) tissue adjacent to the cardiac rhythm management device; (b) the vicinity of the cardiac rhythm management device-tissue interface; (c) the region around the cardiac rhythm management device; and (d) tissue surrounding the cardiac rhythm management device. Methods for infiltrating the anti-fibrosis drug combinations, or individual component(s) thereof, or pharmaceutical compositions that comprise the anti-fibrosis drug combinations, or individual component(s) thereof, into tissue adjacent to a cardiac rhythm management device include delivering the composition: (a) to the surface of the cardiac rhythm management device (e.g., as an injectable, paste, gel or mesh) during the implantation procedure; (b) to the surface of the tissue (e.g., as an injectable, paste, gel, in situ forming gel or mesh) immediately prior to, or during, implantation of the cardiac rhythm management device; (c) to the surface of the cardiac rhythm management device and/or the tissue surrounding the implanted cardiac rhythm management device (e.g., as an injectable, paste, gel, in situ forming gel or mesh) immediately after the implantation of the cardiac rhythm management device; (d) by topical application of the composition into the anatomical space where the cardiac rhythm management device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cardiac rhythm management device as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to cardiac rhythm management devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As cardiac rhythm management devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μm/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

For greater clarity, several specific cardiac rhythm management devices and treatments will be described in greater detail below.

(1) Cardiac Pacemakers

Cardiac pacemakers may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such cardiac pacemakers are provided above, together with methods for coating such devices. Numerous polymeric and non-polymeric delivery systems for use in connection with cardiac pacemakers have been described above.

Anti-fibrosis drug combinations, or individual component(s) thereof, or pharmaceutical compositions that comprise anti-fibrosis drug combinations, or individual component(s) thereof, may be infiltrated around implanted cardiac pacemakers by applying the composition directly and/or indirectly into and/or onto (a) tissue adjacent to the cardiac pacemaker; (b) the vicinity of the cardiac pacemaker-tissue interface; (c) the region around the cardiac pacemaker; and (d) tissue surrounding the cardiac pacemaker. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a cardiac pacemaker include delivering the composition: (a) to the surface of the cardiac pacemaker (e.g., as an injectable, paste, gel or mesh) during the implantation procedure; (b) to the surface of the tissue (e.g., as an injectable, paste, gel, in situ forming gel or mesh) immediately prior to, or during, implantation of the cardiac pacemaker; (c) to the surface of the cardiac pacemaker and/or the tissue surrounding the implanted cardiac pacemaker (e.g., as an injectable, paste, gel, in situ forming gel or mesh) immediately after the implantation of the cardiac pacemaker; (d) by topical application of the composition into the anatomical space where the cardiac pacemaker may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cardiac pacemaker as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to cardiac pacemakers may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As cardiac pacemakers are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g. doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(2) Implantable Cardioverter Defibrillator (ICD) Systems

Implantable cardioverter defibrillator (ICD) systems may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such ICD systems are provided above, together with methods for coating such devices. Numerous polymeric and non-polymeric delivery systems for use in connection with ICD systems have been described above.

Anti-fibrosis drug combinations, or individual component(s) thereof, or pharmaceutical compositions that comprise anti-fibrosis drug combinations, or individual component(s) thereof, may be infiltrated around implanted ICDs by applying the composition directly and/or indirectly into and/or onto (a) tissue adjacent to the ICD; (b) the vicinity of the ICD-tissue interface; (c) the region around the ICD; and (d) tissue surrounding the ICD. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a ICD include delivering the composition: (a) to the surface of the ICD (e.g., as an injectable, paste, gel or mesh) during the implantation procedure; (b) to the surface of the tissue (e.g., as an injectable, paste, gel, in situ forming gel or mesh) immediately prior to, or during, implantation of the ICD; (c) to the surface of the ICD and/or the tissue surrounding the implanted ICD (e.g., as an injectable, paste, gel, in situ forming gel or mesh) immediately after the implantation of the ICD; (d) by topical application of the composition into the anatomical space where the ICD may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the ICD as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to ICDs may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As ICDs are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-250 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(3) Vagus Nerve Stimulation for the Treatment of Arrhythmia

Vagus nerve stimulation devices for the treatment of arrhythmia may benefit from having an anti-scarring drug combination, or individual component(s) thereof, or a composition comprising an anti-scarring drug composition, or individual component(s) thereof, infiltrated into tissue adjacent to where the device and/or leads are or will be implanted according to the present invention. Representative examples of such vagus nerve stimulation devices for the treatment of arrhythmia are provided above, together with methods for coating such devices. Numerous polymeric and non-polymeric delivery systems for use in connection with vagus nerve stimulation devices for the treatment of arrhythmia have been described above.

Anti-fibrosis (or anti-gliosis) drug combinations, or individual component(s) thereof, or pharmaceutical compositions that comprise anti-fibrosis (or anti-gliosis) drug combinations, or individual component(s) thereof, may be infiltrated around implanted VNS devices by applying the composition directly and/or indirectly into and/or onto (a) tissue adjacent to the VNS device; (b) the vicinity of the VNS device-tissue interface; (c) the region around the VNS device; and (d) tissue surrounding the VNS device. Methods for infiltrating the anti-fibrosis (or anti-gliosis) drug combinations, or individual component(s) thereof, or pharmaceutical compositions that comprise the anti-fibrosis (or anti-gliosis) drug combinations, or individual component(s) thereof, into tissue adjacent to a VNS device include delivering the composition: (a) to the surface of the VNS device (e.g., as an injectable, paste, gel or mesh) during the implantation procedure; (b) to the surface of the tissue (e.g., as an injectable, paste, gel, in situ forming gel or mesh) immediately prior to, or during, implantation of the VNS device; (c) to the surface of the VNS device and/or the tissue surrounding the implanted VNS device (e.g., as an injectable, paste, gel, in situ forming gel or mesh) immediately after the implantation of the VNS device; (d) by topical application of the composition into the anatomical space where the VNS device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the VNS device as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the anti-fibrosis (or anti-gliosis) drug combinations, or individual component(s) thereof, or pharmaceutical compositions that comprise the anti-fibrosis (or anti-gliosis) drug combinations, or individual component(s) thereof, may be infiltrated into tissue adjacent to all or a portion of the device.

According to one aspect, any fibrosis-inhibiting (or gliosis-inhibiting) drug combination, or individual component(s) thereof, or pharmaceutical composition that comprises the fibrosis-inhibiting (or gliosis-inhibiting) drug combination, or individual component(s) thereof, described above may be utilized in the practice of the present invention. In one aspect of the invention, the subject compositions infiltrated into tissue adjacent to VNS devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting (or gliosis-inhibiting) drug combinations for use in the present invention include, but are not limited to, the following: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, and terbinafine and manganese sulfate.

The drug dose administered from the anti-fibrosis (or anti-gliosis) drug combinations, or individual component(s) thereof, or pharmaceutical compositions that comprise the anti-fibrosis (or anti-gliosis) drug combinations, or individual component(s) thereof, for prevention or inhibition of fibrosis in accordance with the present invention will depend on a variety of factors, including the type of formulation, the location of the treatment site, and the type of condition being treated. As VNS devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosis (or anti-gliosis) drug combinations, or individual component(s) thereof, or pharmaceutical compositions that comprise the anti-fibrosis (or anti-gliosis) drug combinations, or individual component(s) thereof, should be administered under the following dosing guidelines. The total amount (dose) of anti-scarring agent(s) in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent(s) per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in combination with an anti-fibrosis (or anti-gliosis) drug combination, or individual component(s) thereof, in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Although numerous cardiac rhythm management (CRM) devices have been described above, all possess similar design features and cause similar unwanted fibrous tissue reactions following implantation and may introduce or promote infection in the area of the implant site. It should be obvious to one of skill in the art that commercial CRM devices not specifically cited above as well as next-generation and/or subsequently developed commercial CRM products are to be anticipated and are suitable for use under the present invention. The CRM device, particularly the lead(s), must be positioned in a very precise manner to ensure that stimulation is delivered to the correct anatomical location within the atrium and/or ventricle. All, or parts, of a CRM device can migrate following surgery, or excessive scar tissue growth can occur around the implant, which can lead to a reduction in the performance of these devices. CRM devices having the subject compositions infiltrated into tissue adjacent to the electrode-tissue interface can be used to increase the efficacy and/or the duration of activity of the implant (particularly for fully-implanted, battery-powered devices). CRM devices may also benefit from release of a therapeutic agent able to prevent or inhibit infection in the vicinity of the implant site. In one aspect, the present invention provides CRM devices having the subject compositions infiltrated into adjacent tissue, where the subject compositions may include an anti-fibrosis (or anti-gliosis) drug combination, or individual component(s) thereof. These compositions can further include one or more fibrosis-inhibiting agents such that the overgrowth of granulation fibrous, or gliotic tissue is inhibited or reduced and/or one or more anti-infective agents such that infection in the vicinity of the implant site is inhibited or prevented.

4) Sustained-Release Preparations of Fibrosis-Inhibiting (or Gliosis-Inhibiting) Drug Combinations or Individual Component(s) Thereof

In certain embodiments, desired fibrosis-inhibiting (or gliosis-inhibiting) drug combinations, or individual component(s) thereof, may be admixed with, blended with, conjugated to, or, otherwise modified to contain a polymer composition (which may be either biodegradable or non-biodegradable), or a non-polymeric composition, in order to release the drug combination, or individual component(s) thereof, over a prolonged period of time. For many of the aforementioned embodiments, localized delivery as well as localized sustained delivery of the fibrosis-inhibiting (or gliosis-inhibiting) drug combination, or individual component(s) thereof, may be required. For example, a desired fibrosis-inhibiting (or gliosis-inhibiting) drug combination, or individual component(s) thereof, may be admixed with, blended with, conjugated to, or otherwise modified to contain a polymeric composition (which may be either biodegradable or non-biodegradable), or non-polymeric composition, in order to release the fibrosis-inhibiting (or gliosis-inhibiting) drug combination, or individual component(s) thereof, over a period of time.

In certain aspects, the polymer composition may include a bioerodible or biodegradable polymer. Representative examples of biodegradable polymer compositions suitable for the delivery of fibrosis-inhibiting (or gliosis-inhibiting) drug combinations, or individual component(s) thereof, include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), degradable polyesters (e.g., polyesters comprising the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one), poly(hydroxyvaleric acid), polydioxanone, poly(ethylene terephthalate), poly(malic acid), poly(tartronic acid), poly(acrylamides), polyanhydrides, polyphosphazenes, poly(amino acids), poly(alkylene oxide)-poly(ester) block copolymers (e.g., X—Y, X—Y—X or Y—X—Y, R—(Y—X)ₙ, R—(X—Y)ₙ where X is a polyalkylene oxide (e.g., poly(ethylene glycol), methoxy poly(ethylene glycol), poly(propylene glycol), block copolymers of poly(ethylene oxide) and poly(propylene oxide) (e.g., PLURONIC and PLURONIC R polymers) and Y is a polyester (e.g., polyester comprising the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one), R is a multifunctional initiator and copolymers as well as blends thereof)) and their copolymers, branched polymers as well as blends thereof. (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986)).

Representative examples of non-degradable polymers suitable for the delivery of fibrosis-inhibiting (or gliosis-inhibiting) drug combinations, or individual component(s) thereof, include poly(ethylene-co-vinyl acetate) (“EVA”) copolymers, silicone rubber, acrylic polymers (polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate) poly(octylcyanoacrylate)), polyethylene, polypropylene, polyamides (nylon 6,6), polyurethanes (e.g., CHRONOFLEX AR and CHRONOFLEX AL (both from CardioTech International, Inc., Woburn, Mass.), BIONATE (Polymer Technology Group, Inc., Emergyville, Calif.), and PELLETHANE (Dow Chemical Company, Midland, Mich.)), poly(ester urethanes), poly(ether urethanes), poly(ester-urea), polyethers (poly(ethylene oxide), poly(propylene oxide), block copolymers based on ethylene oxide and propylene oxide (i.e., copolymers of ethylene oxide and propylene oxide polymers), such as the family of PLURONIC polymers available from BASF Corporation (Mount Olive, N.J.), and poly(tetramethylene glycol)), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends thereof (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995).

Particularly preferred polymeric carriers include poly(ethylene-co-vinyl acetate), polyurethanes (e.g., CHRONOFLEX AR, CHRONOFLEX AL, BIONATE, PELLETHANE), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly (caprolactone) or poly(lactic acid) with a polyethylene glycol (e.g., MePEG), silicone rubbers, nitrocellulose, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers.

Other representative polymers capable of sustained localized delivery of fibrosis-inhibiting (or gliosis-inhibiting) drug combinations, or individual component(s) thereof, include carboxylic polymers, polyacetates, polyacrylamides, polycarbonates, polyethers, polyesters, polyethylenes, polyvinylbutyrals, polysilanes, polyureas, polyurethanes, polyurethanes (e.g., CHRONOFLEX AR, CHRONOFLEX AL, BIONATE, AND PELLETHANE), polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, polyvinylchloride acetate.

In one embodiment, all or a portion of the device is coated with a primer (bonding) layer and a drug release layer, as described in U.S. patent application entitled, “Stent with Medicated Multi-Layer Hybrid Polymer Coating,” filed Sep. 16, 2003 (U.S. Ser. No. 10/662,877).

In order to develop a hybrid polymer delivery system for targeted therapy, it is desirable to be able to control and manipulate the properties of the system both in terms of physical and drug release characteristics. The active drug combinations, or individual component(s) thereof, can be imbibed into a surface hybrid polymer layer, or incorporated directly into the hybrid polymer coating solutions. Imbibing drugs into surface polymer layers is an efficient method for evaluating polymer-drug performance in the laboratory, but for commercial production it may be preferred for the polymer and drugs to be premixed in the casting mixture. Greater efficacy can be achieved by combining the two elements in the coating mixtures in order to control the ratio of active agent to polymer in the coatings. Such ratios are important parameters to the final properties of the medicated layers, i.e., they allow for better control of active agent concentration and duration of pharmacological activity.

Typical polymers used in the drug-release system can include water-insoluble cellulose esters, various polyurethane polymers including hydrophilic and hydrophobic versions, hydrophilic polymers such as polyethylene glycol (PEG), polyethylene oxide (PEO), polyvinylpyrrolidone (PVP), PVP copolymers such as vinyl acetate, hydroxyethyl methacrylate (HEMA) and copolymers such as methylmethacrylate (PMMA-HEMA), and other hydrophilic and hydrophobic acrylate polymers and copolymers containing functional groups such as carboxyl and/or hydroxyl.

Cellulose esters such as cellulose acetate, cellulose acetate propionate, cellulose acetate butyrate, cellulose acetate phthalate, and cellulose nitrate may be used. In one aspect of the invention, the drug combination, or individual component(s) thereof, is formulated with a cellulose ester. Cellulose nitrate is a preferred cellulose ester because of its compatibility with the active agents and its ability to impart non-tackiness and cohesiveness to the coatings. Cellulose nitrate has been shown to stabilize entrapped drugs in ambient and processing conditions. Various grades of cellulose nitrate are available and may be used in a coating on a electrical device, including cellulose nitrate having a nitrogen content=11.8-12.2%. Various viscosity grades, including 3.5, 0.5 or 0.25 seconds, may be used in order to provide proper rheological properties when combined with the coating solids used in these formulations. Higher or lower viscosity grades can be used. However, the higher viscosity grades can be more difficult to use because of their higher viscosities. Thus, the lower viscosity grades, such as 3.5, 0.5 or 0.25 seconds, are generally preferred. Physical properties such as tensile strength, elongation, flexibility, and softening point are related to viscosity (molecular weight) and can decrease with the lower molecular weight species, especially below the 0.25-second grades.

The cellulose derivatives comprise hydroglucose structures. Cellulose nitrate is a hydrophobic, water-insoluble polymer, and has high water resistance properties. This structure leads to high compatibility with many active agents, accounting for the high degree of stabilization provided to drugs entrapped in cellulose nitrate. The structure of nitrocellulose is given below: 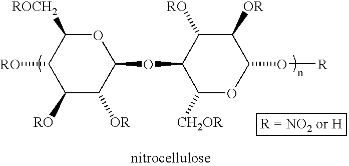
Cellulose nitrate is a hard, relatively inflexible polymer, and has limited adhesion to many polymers that are typically used to make medical devices. Also, control of drug elution dynamics is limited if only one polymer is used in the binding matrix. Accordingly, in one embodiment of the invention, the drug combination, or individual component(s) thereof, is formulated with two or more polymers before being associated with the electrical device. In one aspect, the agent is formulated with both polyurethane ((e.g., CHRONOFLEX AR, CHRONOFLEX AL, BIONATE, and PELLETHANE) and cellulose nitrate to provide a hybrid polymer drug loaded matrix. Polyurethanes provide the hybrid polymer matrix with greater flexibility and adhesion to the electrical device, particularly when the connector has been pre-coated with a primer. Polyurethanes can also be used to slow or hasten the drug elution from coatings. Aliphatic, aromatic, polytetramethylene ether glycol, and polycarbonate are among the types of polyurethanes, which can be used in the coatings. In one aspect, an anti-scarring drug combination, or individual component(s) thereof, may be incorporated into a carrier that includes a polyurethane and a cellulose derivative. A heparin complex, such as benzalkonium heparinate or tridodecylammonium heparinate), may optionally be included in the formulation.

From the structure below, it is possible to see how more or less hydrophilic polyurethane polymers may be created based on the number of hydrophilic groups contained in the polymer structures. In one aspect of the invention, the electrical device is associated with a formulation that includes therapeutic agent, cellulose ester, and a polyurethane that is water-insoluble, flexible, and compatible with the cellulose ester.

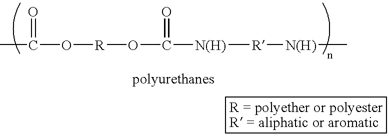

Polyvinylpyrrolidone (PVP) is a polyamide that possesses unusual complexing and colloidal properties and is essentially physiologically inert. PVP and other hydrophilic polymers are typically biocompatible. PVP may be incorporated into drug-loaded hybrid polymer compositions in order to increase release rates of drug combination(s), or individual component(s) thereof. In one embodiment, the concentration of PVP that is used in drug loaded hybrid polymer compositions can be less than 20%. This concentration cannot make the layers bioerodable or lubricious. In general, PVP concentrations from <1% to greater than 80% are deemed workable. In one aspect of the invention, the drug combination, or individual component(s) thereof, that is associated with an electrical device is formulated with a PVP polymer.

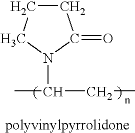

Acrylate polymers and copolymers including polymethylmethacrylate (PMMA) and polymethylmethacrylate hydroxyethyl methacrylate (PMMA/HEMA) are known for their biocompatibility as a result of their widespread use in contact and intraocular lens applications. This class of polymer generally provokes very little smooth muscle and endothelial cell growth, and very low inflammatory response (Bar). These polymers/copolymers are compatible with drugs and the other polymers and layers of the instant invention. Thus, in one aspect, the device is associated with a composition that comprises an anti-scarring drug combination, or individual component(s) thereof, as described above, and an acrylate polymer or copolymer.

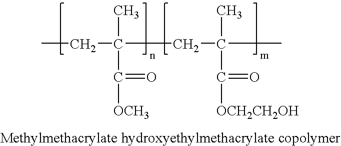

Representative examples of patents relating to drug-delivery polymers and their preparation include PCT Publication Nos. WO 98/19713, WO 01/17575, WO 01/41821, WO 01/41822, and WO 01/15526 (as well as their corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE 37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, and 5,714,159, 5,612,052 and U.S. Patent Application Publication Nos. 2003/0068377, 2002/0192286, 2002/0076441, and 2002/0090398.

It should be obvious to one of skill in the art that the polymers as described herein can also be blended or copolymerized in various compositions as required to deliver therapeutic doses of fibrosis-inhibiting (or gliosis-inhibiting) drug combinations, or individual component(s) thereof.

Polymeric carriers for fibrosis-inhibiting (or gliosis-inhibiting) drug combinations, or individual component(s) thereof, can be fashioned in a variety of forms, with desired release characteristics and/or with specific properties depending upon the device, composition or implant being utilized. For example, polymeric carriers may be fashioned to release a fibrosis-inhibiting (or gliosis-inhibiting) drug combination, or individual component(s) thereof, upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide monomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, fibrosis-inhibiting (or gliosis-inhibiting) drug combinations, or individual components thereof, can be delivered via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and their gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y and X—Y—X where X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and their preparation include U.S. Pat. Nos. 6,451,346; 6,201,072; 6,117,949; 6,004,573; 5,702,717; and 5,484,610 and PCT Publication Nos. WO 99/07343; WO 99/18142; WO 03/17972; WO 01/82970; WO 00/18821; WO 97/15287; WO 01/41735; WO 00/00222 and WO 00/38651.

Fibrosis-inhibiting (or gliosis-inhibiting) drug combinations, or individual component(s) thereof, may be linked by occlusion in the matrices of the polymer, bound by covalent linkages, or encapsulated in microcapsules. Within certain embodiments of the invention, therapeutic compositions are provided in non-capsular formulations such as microspheres (ranging from nanometers to micrometers in size), pastes, threads of various size, films and sprays.

Within certain aspects of the present invention, therapeutic compositions of anti-scarring drug combinations may be fashioned into particles having any size ranging from 50 nm to 500 μm, depending upon the particular use. These compositions can be in the form of microspheres, microparticles and/or nanoparticles. These compositions can be formed by spray-drying methods, milling methods, coacervation methods, W/O emulsion methods, W/O/W emulsion methods, and solvent evaporation methods. In another embodiment, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention that include anti-scarring drug combinations may also be prepared in a variety of paste or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing fibrosis-inhibiting agents are particularly useful for application to the surface of tissues that will be in contact with the implant or device.

Within yet other aspects of the invention, the therapeutic compositions of the present invention may be formed as a film or tube. These films or tubes can be porous or non-porous. Such films or tubes are generally less than 5, 4, 3, 2, or 1 mm thick, or less than 0.75 mm, or less than 0.5 mm, or less than 0.25 mm, or, less than 0.10 mm thick. Films or tubes can also be generated of thicknesses less than 50 μm, 25 μm or 10 μm. Such films may be flexible with a good tensile strength (e.g., greater than 50, or greater than 100, or greater than 150 or 200 N/cm²), good adhesive properties (i.e., adheres to moist or wet surfaces), and have controlled permeability. Fibrosis-inhibiting (or gliosis-inhibiting) drug combinations, or individual component(s) thereof, contained in polymeric films are particularly useful for application to the surface of a device or implant as well as to the surface of tissue, cavity or an organ.

Within further aspects of the present invention, polymeric carriers are provided which are adapted to contain and release a hydrophobic fibrosis-inhibiting (or gliosis-inhibiting) drug combination, or individual component(s) thereof. In certain embodiments, the carriers that contain and release the hydrophobic drug combination, or individual component(s) thereof, are further in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix which contains the hydrophobic fibrosis-inhibiting (or gliosis-inhibiting) drug combination, or individual component(s) thereof, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan, hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver fibrosis-inhibiting (or gliosis-inhibiting) drug combinations, or individual component(s) thereof, described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2): 191-197, 1990), micelles (Alkan-Onyuksel et al., Pharm. Res. 11(2):206-212, 1994), implants (Jampel et al., Invest. Ophthalm. Vis. Science 34(11):3076-3083, 1993; Walter et al., Cancer Res. 54:22017-2212, 1994), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

Within another aspect of the present invention, polymeric carriers can be materials that are formed in situ. In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible light, UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 00/64977, U.S. Pat. Nos. 5,900,245, 6,051,248, 6,083,524, 6,177,095, 6,201,065, 6,217,894, 6,639,014, 6,352,710, 6,410,645, 6,531,147, 5,567,435, 5,986,043, 6,602,975, and U.S. Patent Application Publication Nos. 2002/012796A1, 2002/0127266A1, 2002/0151650A1, 2003/0104032A1, 2002/0091229A1, and 2003/0059906A1.

As mentioned elsewhere herein, the present invention provides for polymeric crosslinked matrices, and polymeric carriers, that may be used to assist in the prevention of the formation or growth of fibrous connective tissue or glial tissue. The composition may contain and deliver fibrosis-inhibiting (or gliosis-inhibiting) drug combinations, or individual component(s) thereof, in the vicinity of the medical device. The following compositions are particularly useful when it is desired to infiltrate around the device. Such polymeric materials may be prepared from, e.g., (a) synthetic materials, (b) naturally-occurring materials, or (c) mixtures of synthetic and naturally occurring materials. The matrix may be prepared from, e.g., (a) a one-component, i.e., self-reactive, compound, or (b) two or more compounds that are reactive with one another. Typically, these materials are fluid prior to delivery, and thus can be sprayed or otherwise extruded from a device in order to deliver the composition. After delivery, the component materials react with each other, and/or with the body, to provide the desired affect. In some instances, materials that are reactive with one another must be kept separated prior to delivery to the patient, and are mixed together just prior to being delivered to the patient, in order that they maintain a fluid form prior to delivery. In a preferred aspect of the invention, the components of the matrix are delivered in a liquid state to the desired site in the body, whereupon in situ polymerization occurs.

First and Second Synthetic Polymers

In one embodiment, crosslinked polymer compositions (in other words, crosslinked matrices) are prepared by reacting a first synthetic polymer containing two or more nucleophilic groups with a second synthetic polymer containing two or more electrophilic groups, where the electrophilic groups are capable of covalently binding with the nucleophilic groups. In one embodiment, the first and second polymers are each non-immunogenic. In another embodiment, the matrices are not susceptible to enzymatic cleavage by, e.g., a matrix metalloproteinase (e.g., collagenase) and are therefore expected to have greater long-term persistence in vivo than collagen-based compositions.

As used herein, the term “polymer” refers inter alia to polyalkyls, polyamino acids, polyalkyleneoxides and polysaccharides. Additionally, for external or oral use, the polymer may be polyacrylic acid or carbopol. As used herein, the term “synthetic polymer” refers to polymers that are not naturally occurring and that are produced via chemical synthesis. As such, naturally occurring proteins such as collagen and naturally occurring polysaccharides such as hyaluronic acid are specifically excluded. Synthetic collagen, and synthetic hyaluronic acid, and their derivatives, are included. Synthetic polymers containing either nucleophilic or electrophilic groups are also referred to herein as “multifunctionally activated synthetic polymers.” The term “multifunctionally activated” (or, simply, “activated”) refers to synthetic polymers which have, or have been chemically modified to have, two or more nucleophilic or electrophilic groups which are capable of reacting with one another (i.e., the nucleophilic groups react with the electrophilic groups) to form covalent bonds. Types of multifunctionally activated synthetic polymers include difunctionally activated, tetrafunctionally activated, and star-branched polymers.

Multifunctionally activated synthetic polymers for use in the present invention must contain at least two, more preferably, at least three, functional groups in order to form a three-dimensional crosslinked network with synthetic polymers containing multiple nucleophilic groups (i.e., “multi-nucleophilic polymers”). In other words, they must be at least difunctionally activated, and are more preferably trifunctionally or tetrafunctionally activated. If the first synthetic polymer is a difunctionally activated synthetic polymer, the second synthetic polymer must contain three or more functional groups in order to obtain a three-dimensional crosslinked network. Most preferably, both the first and the second synthetic polymer contain at least three functional groups.

Synthetic polymers containing multiple nucleophilic groups are also referred to generically herein as “multi-nucleophilic polymers.” For use in the present invention, multi-nucleophilic polymers must contain at least two, more preferably, at least three, nucleophilic groups. If a synthetic polymer containing only two nucleophilic groups is used, a synthetic polymer containing three or more electrophilic groups must be used in order to obtain a three-dimensional crosslinked network.

Preferred multi-nucleophilic polymers for use in the compositions and methods of the present invention include synthetic polymers that contain, or have been modified to contain, multiple nucleophilic groups such as primary amino groups and thiol groups. Preferred multi-nucleophilic polymers include: (i) synthetic polypeptides that have been synthesized to contain two or more primary amino groups or thiol groups; and (ii) polyethylene glycols that have been modified to contain two or more primary amino groups or thiol groups. In general, reaction of a thiol group with an electrophilic group tends to proceed more slowly than reaction of a primary amino group with an electrophilic group.

In one embodiment, the multi-nucleophilic polypeptide is a synthetic polypeptide that has been synthesized to incorporate amino acid residues containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000.

Poly(lysine)s for use in the present invention preferably have a molecular weight within the range of about 1,000 to about 300,000; more preferably, within the range of about 5,000 to about 100,000; most preferably, within the range of about 8,000 to about 15,000. Poly(lysine)s of varying molecular weights are commercially available from Peninsula Laboratories, Inc. (Belmont, Calif.) and Aldrich Chemical (Milwaukee, Wis.).

Polyethylene glycol can be chemically modified to contain multiple primary amino or thiol groups according to methods set forth, for example, in Chapter 22 of Poly(ethylene Glycol) Chemistry: Biotechnical and Biomedical Applications, J. Milton Harris, ed., Plenum Press, N.Y. (1992). Polyethylene glycols which have been modified to contain two or more primary amino groups are referred to herein as “multi-amino PEGs.” Polyethylene glycols which have been modified to contain two or more thiol groups are referred to herein as “multi-thiol PEGs.” As used herein, the term “polyethylene glycol(s)” includes modified and or derivatized polyethylene glycol(s).

Various forms of multi-amino PEG are commercially available from Shearwater Polymers (Huntsville, Ala.) and from Huntsman Chemical Company (Utah) under the name “Jeffamine.” Multi-amino PEGs useful in the present invention include Huntsman's Jeffamine diamines (“D” series) and triamines (“T” series), which contain two and three primary amino groups per molecule, respectively.

Polyamines such as ethylenediamine (H₂N—CH₂—CH₂—NH₂), tetramethylenediamine (H₂N—(CH₂)₄—NH₂), pentamethylenediamine (cadaverine) (H₂N—(CH₂)₅—NH₂), hexamethylenediamine (H₂N—(CH₂)₆—NH₂), di(2-aminoethyl)amine (HN—(CH₂—CH₂—NH₂)₂), and tris(2-aminoethyl)amine (N—(CH₂—CH₂—NH₂)₃) may also be used as the synthetic polymer containing multiple nucleophilic groups.

Synthetic polymers containing multiple electrophilic groups are also referred to herein as “multi-electrophilic polymers.” For use in the present invention, the multifunctionally activated synthetic polymers must contain at least two, more preferably, at least three, electrophilic groups in order to form a three-dimensional crosslinked network with multi-nucleophilic polymers. Preferred multi-electrophilic polymers for use in the compositions of the invention are polymers which contain two or more succinimidyl groups capable of forming covalent bonds with nucleophilic groups on other molecules. Succinimidyl groups are highly reactive with materials containing primary amino (NH₂) groups, such as multi-amino PEG, poly(lysine), or collagen. Succinimidyl groups are slightly less reactive with materials containing thiol (SH) groups, such as multi-thiol PEG or synthetic polypeptides containing multiple cysteine residues.

As used herein, the term “containing two or more succinimidyl groups” is meant to encompass polymers which are preferably commercially available containing two or more succinimidyl groups, as well as those that must be chemically derivatized to contain two or more succinimidyl groups. As used herein, the term “succinimidyl group” is intended to encompass sulfosuccinimidyl groups and other such variations of the “generic” succinimidyl group. The presence of the sodium sulfite moiety on the sulfosuccinimidyl group serves to increase the solubility of the polymer.

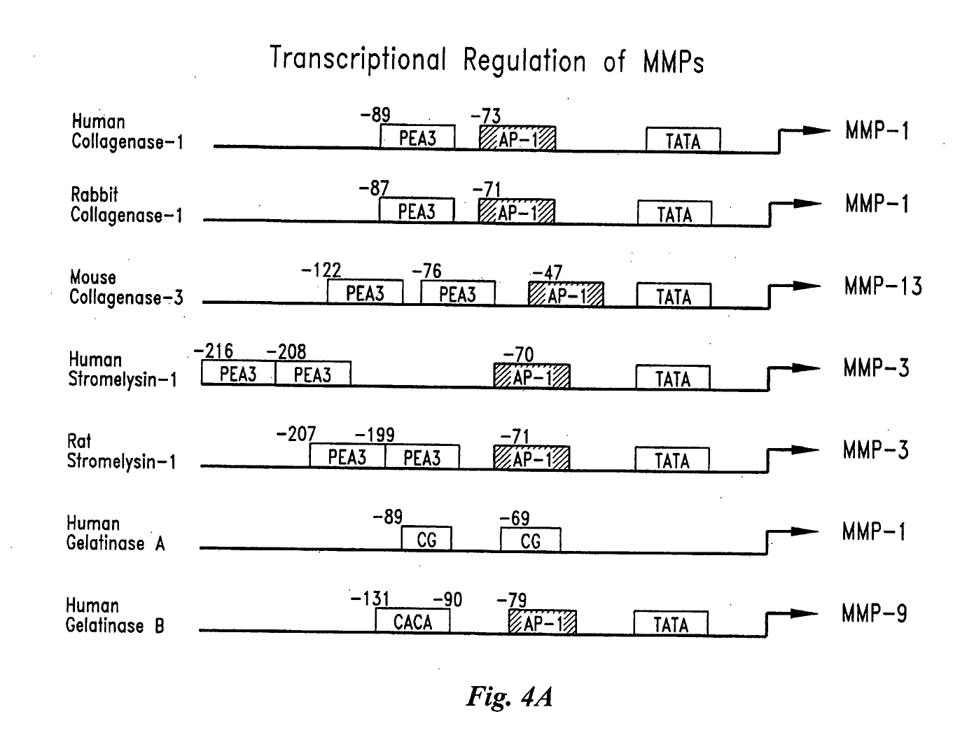

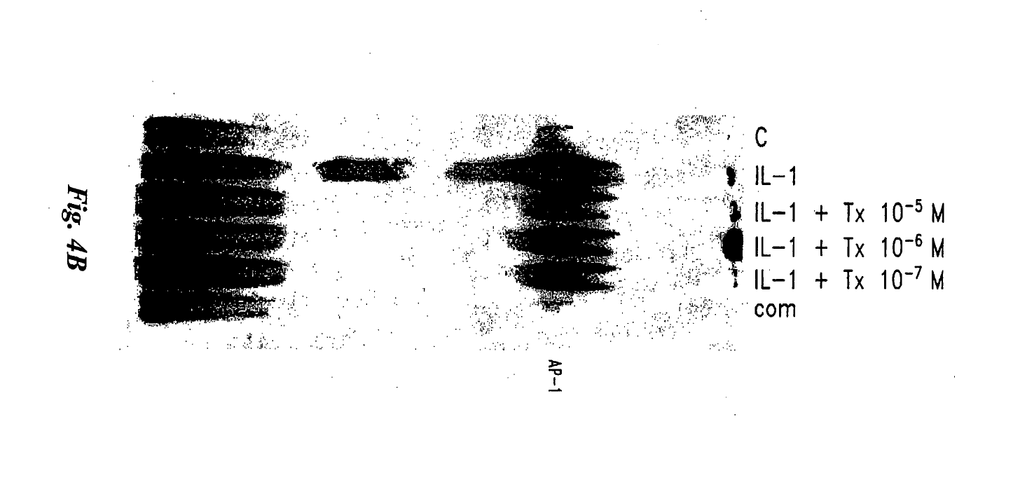

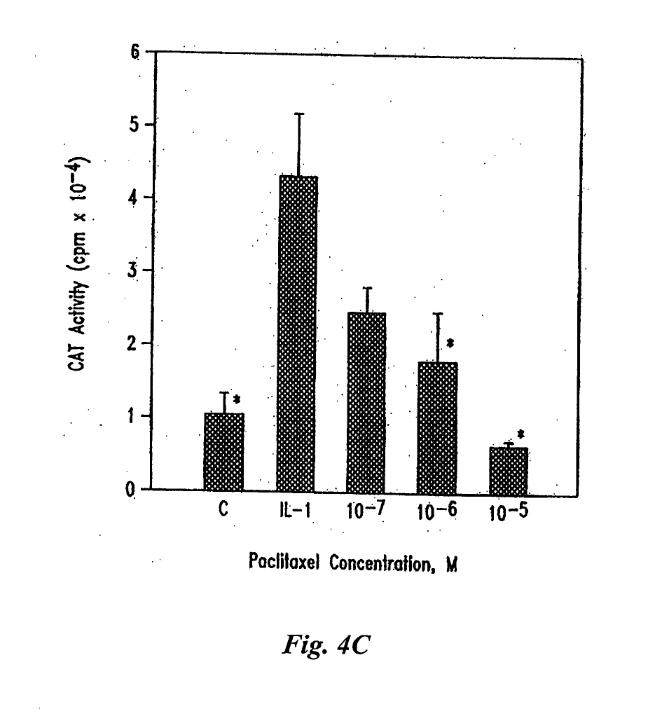

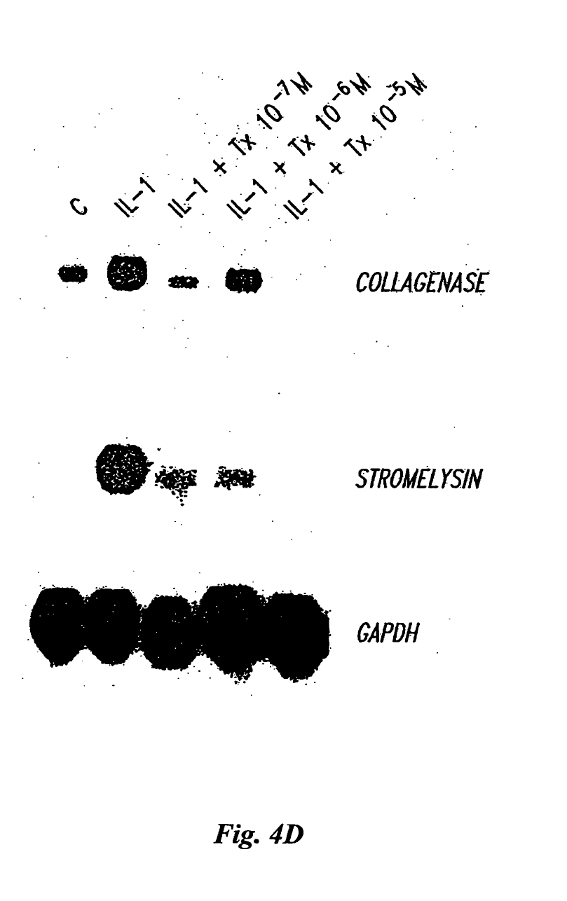

Hydrophilic polymers and, in particular, various derivatized polyethylene glycols, are preferred for use in the compositions of the present invention. As used herein, the term “PEG” refers to polymers having the repeating structure (OCH₂—CH₂)ₙ. Structures for some specific, tetrafunctionally activated forms of PEG are shown in FIGS. 4 to 13 of U.S. Pat. No. 5,874,500, incorporated herein by reference. Examples of suitable PEGS include PEG succinimidyl propionate (SE-PEG), PEG succinimidyl succinamide (SSA-PEG), and PEG succinimidyl carbonate (SC-PEG). In one aspect of the invention, the crosslinked matrix is formed in situ by reacting pentaerythritol poly(ethylene glycol)ether tetra-sulfhydryl] (4-armed thiol PEG) and pentaerythritol poly(ethylene glycol)ether tetra-succinimidyl glutarate] (4-armed NHS PEG) as reactive reagents. Structures for these reactants are shown in U.S. Pat. No. 5,874,500. Each of these materials has a core with a structure that may be seen by adding ethylene oxide-derived residues to each of the hydroxyl groups in pentaerythritol, and then derivatizing the terminal hydroxyl groups (derived from the ethylene oxide) to contain either thiol groups (so as to form 4-armed thiol PEG) or N-hydroxysuccinimydyl groups (so as to form 4-armed NHS PEG), optionally with a linker group present between the ethylene oxide derived backbone and the reactive functional group, where this product is commercially available as COSEAL from Angiotech Pharmaceuticals Inc. Optionally, a group “D” may be present in one or both of these molecules, as discussed in more detail below.

As discussed above, preferred activated polyethylene glycol derivatives for use in the invention contain succinimidyl groups as the reactive group. However, different activating groups can be attached at sites along the length of the PEG molecule. For example, PEG can be derivatized to form functionally activated PEG propionaldehyde (A-PEG), or functionally activated PEG glycidyl ether (E-PEG), or functionally activated PEG-isocyanate (1-PEG), or functionally activated PEG-vinylsulfone (V-PEG).

Hydrophobic polymers can also be used to prepare the compositions of the present invention. Hydrophobic polymers for use in the present invention preferably contain, or can be derivatized to contain, two or more electrophilic groups, such as succinimidyl groups, most preferably, two, three, or four electrophilic groups. As used herein, the term “hydrophobic polymer” refers to polymers which contain a relatively small proportion of oxygen or nitrogen atoms.

Hydrophobic polymers which already contain two or more succinimidyl groups include, without limitation, disuccinimidyl suberate (DSS), bis(sulfosuccinimidyl) suberate (BS3), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives. The above-referenced polymers are commercially available from Pierce (Rockford, Ill.), under catalog Nos. 21555, 21579, 22585, 21554, and 21577, respectively.

Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing multiple nucleophilic groups.

Certain polymers, such as polyacids, can be derivatized to contain two or more functional groups, such as succinimidyl groups. Polyacids for use in the present invention include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid). Many of these polyacids are commercially available from DuPont Chemical Company (Wilmington, Del.). According to a general method, polyacids can be chemically derivatized to contain two or more succinimidyl groups by reaction with an appropriate molar amount of N-hydroxysuccinimide (NHS) in the presence of N,N′-dicyclohexylcarbodiimide (DCC).

Polyalcohols such as trimethylolpropane and di(trimethylol propane) can be converted to carboxylic acid form using various methods, then further derivatized by reaction with NHS in the presence of DCC to produce trifunctionally and tetrafunctionally activated polymers, respectively, as described in U.S. application Ser. No. 08/403,358. Polyacids such as heptanedioic acid (HOOC—(CH₂)₅—COOH), octanedioic acid (HOOC—(CH₂)₆—COOH), and hexadecanedioic acid (HOOC—(CH₂)₁₄—COOH) are derivatized by the addition of succinimidyl groups to produce difunctionally activated polymers.

Polyamines such as ethylenediamine, tetramethylenediamine, pentamethylenediamine (cadaverine), hexamethylenediamine, bis(2-aminoethyl)amine, and tris(2-aminoethyl)amine can be chemically derivatized to polyacids, which can then be derivatized to contain two or more succinimidyl groups by reacting with the appropriate molar amounts of N-hydroxysuccinimide in the presence of DCC, as described in U.S. application Ser. No. 08/403,358. Many of these polyamines are commercially available from DuPont Chemical Company.

In a preferred embodiment, the first synthetic polymer will contain multiple nucleophilic groups (represented below as “X”) and it will react with the second synthetic polymer containing multiple electrophilic groups (represented below as “Y”), resulting in a covalently bound polymer network, as follows:
Polymer-Xₘ+Polymer-Yₙ→Polymer-Z-Polymer

wherein m≦2, n≦2, and m+n≦5;

where exemplary X groups include —NH₂, —SH, —OH, —PH₂, CO—NH—NH₂, etc., where the X groups may be the same or different in polymer-Xₘ;

where exemplary Y groups include —CO₂—N(COCH₂)₂, —CO₂H, —CHO, —CHOCH₂ (epoxide), —N═C═O, —SO₂—CH═CH₂, —N(COCH)₂ (i.e., a five-membered heterocyclic ring with a double bond present between the two CH groups), —S—S—(C₅H₄N), etc., where the Y groups may be the same or different in polymer-Yₙ; and

where Z is the functional group resulting from the union of a nucleophilic group (X) and an electrophilic group (Y).

As noted above, it is also contemplated by the present invention that X and Y may be the same or different, i.e., a synthetic polymer may have two different electrophilic groups, or two different nucleophilic groups, such as with glutathione.

In one embodiment, the backbone of at least one of the synthetic polymers comprises alkylene oxide residues, e.g., residues from ethylene oxide, propylene oxide, and mixtures thereof. The term ‘backbone’ refers to a significant portion of the polymer.

For example, the synthetic polymer containing alkylene oxide residues may be described by the formula X-polymer-X or Y-polymer-Y, wherein X and Y are as defined above, and the term “polymer” represents —(CH₂CH₂O)ₙ— or —(CH(CH₃)CH₂O)ₙ— or —(CH₂—CH₂—O)ₙ—(CH(CH₃)CH₂—O)ₙ—. In these cases the synthetic polymer would be difunctional.

The required functional group X or Y is commonly coupled to the polymer backbone by a linking group (represented below as “Q”), many of which are known or possible. There are many ways to prepare the various functionalized polymers, some of which are listed below:
Polymer-Q₁-X+Polymer-Q₂-Y→Polymer-Q₁-Z-Q₂-Polymer

Exemplary Q groups include —O—(CH₂)ₙ—; —S—(CH₂)ₙ—; —NH—(CH₂)ₙ—; —O₂C—NH—(CH₂)ₙ—; —O₂C—(CH₂)ₙ—; —O₂C—(CR¹H)ₙ—; and —O—R₂—CO—NH—, which provide synthetic polymers of the partial structures: polymer-O—(CH₂)ₙ—(X or Y); polymer-S—(CH₂)ₙ—(X or Y); polymer-NH—(CH₂)ₙ—(X or Y); polymer-O₂C—NH—(CH₂)ₙ—(X or Y); polymer-O₂C—(CH₂)ₙ—(X or Y); polymer-O₂C—(CR¹H)ₙ—(X or Y); and polymer-O—R₂—CO—NH—(X or Y), respectively. In these structures, n=1-10, R¹=H or alkyl (i.e., CH₃, C₂H₅, etc.); R²=CH₂, or CO—NH—CH₂CH₂; and Q₁ and Q₂ may be the same or different.

For example, when Q₂=OCH₂CH₂ (there is no Q₁ in this case); Y=—CO₂—N(COCH₂)₂; and X=NH₂, —SH, or —OH, the resulting reactions and Z groups would be as follows:
Polymer-NH₂+Polymer-O—CH₂—CH₂—CO₂—N(COCH₂)₂→Polymer-NH—CO—CH₂—CH₂—O-Polymer;
Polymer-SH+Polymer-O—CH₂—CH₂—CO₂—N(COCH₂)₂→Polymer-S—COCH₂CH₂—O-Polymer; and
Polymer-OH+Polymer-O—CH₂—CH₂—CO₂—N(COCH₂)₂→Polymer-O—COCH₂CH₂—O-Polymer.

An additional group, represented below as “D”, can be inserted between the polymer and the linking group, if present. One purpose of such a D group is to affect the degradation rate of the crosslinked polymer composition in vivo, for example, to increase the degradation rate, or to decrease the degradation rate. This may be useful in many instances, for example, when drug has been incorporated into the matrix, and it is desired to increase or decrease polymer degradation rate so as to influence a drug delivery profile in the desired direction. An illustration of a crosslinking reaction involving first and second synthetic polymers each having D and Q groups is shown below.
Polymer-D-Q-X+Polymer-D-Q-Y→Polymer-D-Q-Z-Q-D-Polymer

Some useful biodegradable groups “D” include polymers formed from one or more α-hydroxy acids, e.g., lactic acid, glycolic acid, and the cyclization products thereof (e.g., lactide, glycolide), ε-caprolactone, and amino acids. The polymers may be referred to as polylactide, polyglycolide, poly(co-lactide-glycolide); poly-ε-caprolactone, polypeptide (also known as poly amino acid, for example, various di- or tri-peptides) and poly(anhydride)s.

In a general method for preparing the crosslinked polymer compositions used in the context of the present invention, a first synthetic polymer containing multiple nucleophilic groups is mixed with a second synthetic polymer containing multiple electrophilic groups. Formation of a three-dimensional crosslinked network occurs as a result of the reaction between the nucleophilic groups on the first synthetic polymer and the electrophilic groups on the second synthetic polymer.

The concentrations of the first synthetic polymer and the second synthetic polymer used to prepare the compositions of the present invention will vary depending upon a number of factors, including the types and molecular weights of the particular synthetic polymers used and the desired end use application. In general, when using multi-amino PEG as the first synthetic polymer, it is preferably used at a concentration in the range of about 0.5 to about 20 percent by weight of the final composition, while the second synthetic polymer is used at a concentration in the range of about 0.5 to about 20 percent by weight of the final composition. For example, a final composition having a total weight of 1 gram (1000 milligrams) would contain between about 5 to about 200 milligrams of multi-amino PEG, and between about 5 to about 200 milligrams of the second synthetic polymer.

Use of higher concentrations of both first and second synthetic polymers will result in the formation of a more tightly crosslinked network, producing a stiffer, more robust gel. Compositions intended for use in tissue augmentation will generally employ concentrations of first and second synthetic polymer that fall toward the higher end of the preferred concentration range. Compositions intended for use as bioadhesives or in adhesion prevention do not need to be as firm and may therefore contain lower polymer concentrations.

Because polymers containing multiple electrophilic groups will also react with water, the second synthetic polymer is generally stored and used in sterile, dry form to prevent the loss of crosslinking ability due to hydrolysis which typically occurs upon exposure of such electrophilic groups to aqueous media. Processes for preparing synthetic hydrophilic polymers containing multiple electrophylic groups in sterile, dry form are set forth in U.S. Pat. No. 5,643,464. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates. In contrast, polymers containing multiple nucleophilic groups are generally not water-reactive and can therefore be stored in aqueous solution.

In certain embodiments, one or both of the electrophilic- or nucleophilic-terminated polymers described above can be combined with a synthetic or naturally occurring polymer. The presence of the synthetic or naturally occurring polymer may enhance the mechanical and/or adhesive properties of the in situ forming compositions. Naturally occurring polymers, and polymers derived from naturally occurring polymer that may be included in in situ forming materials include naturally occurring proteins, such as collagen, collagen derivatives (such as methylated collagen), fibrinogen, thrombin, albumin, fibrin, and derivatives of and naturally occurring polysaccharides, such as glycosaminoglycans, including deacetylated and desulfated glycosaminoglycan derivatives.

In certain embodiments, a composition comprising naturally occurring protein and both of the first and second synthetic polymers, as described above, is used to form the crosslinked matrix according to the present invention. In certain embodiments, a composition comprising collagen and both of the first and second synthetic polymer as described above is used to form the crosslinked matrix according to the present invention. In certain embodiments, a composition comprising methylated collagen and both of the first and second synthetic polymer as described above is used to form the crosslinked matrix according to the present invention. In certain embodiments, a composition comprising fibrinogen and both of the first and second synthetic polymer as described above is used to form the crosslinked matrix according to the present invention. In certain embodiments, a composition comprising thrombin and both of the first and second synthetic polymer as described above is used to form the crosslinked matrix according to the present invention. In certain embodiments, a composition comprising albumin and both of the first and second synthetic polymer as described above is used to form the crosslinked matrix according to the present invention. In certain embodiments, a composition comprising fibrin and both of the first and second synthetic polymer as described above is used to form the crosslinked matrix according to the present invention. In certain embodiments, a composition comprising naturally occurring polysaccharide and both of the first and second synthetic polymer as described above is used to form the crosslinked matrix according to the present invention. In certain embodiments, a composition comprising glycosaminoglycan and both of the first and second synthetic polymer as described above is used to form the crosslinked matrix according to the present invention. In certain embodiments, a composition comprising deacetylated glycosaminoglycan and both of the first and second synthetic polymer as described above is used to form the crosslinked matrix according to the present invention. In certain embodiments, a composition comprising desulfated glycosaminoglycan and both of the first and second synthetic polymer as described above is used to form the crosslinked matrix according to the present invention.

In certain embodiments, a composition comprising naturally-occurring protein and the first synthetic polymer as described above is used to form the crosslinked matrix according to the present invention. In certain embodiments, a composition comprising collagen and the first synthetic polymer as described above is used to form the crosslinked matrix according to the present invention. In certain embodiments, a composition comprising methylated collagen and the first synthetic polymer as described above is used to form the crosslinked matrix according to the present invention. In certain embodiments, a composition comprising fibrinogen and the first synthetic polymer as described above is used to form the crosslinked matrix according to the present invention. In certain embodiments, a composition comprising thrombin and the first synthetic polymer as described above is used to form the crosslinked matrix according to the present invention. In certain embodiments, a composition comprising albumin and the first synthetic polymer as described above is used to form the crosslinked matrix according to the present invention. In certain embodiments, a composition comprising fibrin and the first synthetic polymer as described above is used to form the crosslinked matrix according to the present invention. In certain embodiments, a composition comprising naturally occurring polysaccharide and the first synthetic polymer as described above is used to form the crosslinked matrix according to the present invention. In certain embodiments, a composition comprising glycosaminoglycan and the first synthetic polymer as described above is used to form the crosslinked matrix according to the present invention. In certain embodiments, a composition comprising deacetylated glycosaminoglycan and the first synthetic polymer as described above is used to form the crosslinked matrix according to the present invention. In certain embodiments, a composition comprising desulfated glycosaminoglycan and the first synthetic polymer as described above is used to form the crosslinked matrix according to the present invention.

In certain embodiments, a composition comprising naturally occurring protein and the second synthetic polymer as described above is used to form the crosslinked matrix according to the present invention. In certain embodiments, a composition comprising collagen and the second synthetic polymer as described above is used to form the crosslinked matrix according to the present invention. In certain embodiments, a composition comprising methylated collagen and the second synthetic polymer as described above is used to form the crosslinked matrix according to the present invention. In certain embodiments, a composition comprising fibrinogen and the second synthetic polymer as described above is used to form the crosslinked matrix according to the present invention. In certain embodiments, a composition comprising thrombin and the second synthetic polymer as described above is used to form the crosslinked matrix according to the present invention. In certain embodiments, a composition comprising albumin and the second synthetic polymer as described above is used to form the crosslinked matrix according to the present invention. In certain embodiments, a composition comprising fibrin and the second synthetic polymer as described above is used to form the crosslinked matrix according to the present invention. In certain embodiments, a composition comprising naturally occurring polysaccharide and the second synthetic polymer as described above is used to form the crosslinked matrix according to the present invention. In certain embodiments, a composition comprising glycosaminoglycan and the second synthetic polymer as described above is used to form the crosslinked matrix according to the present invention. In certain embodiments, a composition comprising deacetylated glycosaminoglycan and the second synthetic polymer as described above is used to form the crosslinked matrix according to the present invention. In certain embodiments, a composition comprising desulfated glycosaminoglycan and the second synthetic polymer as described above is used to form the crosslinked matrix according to the present invention.

The presence of protein or polysaccharide components which contain functional groups that can react with the functional groups on multiple activated synthetic polymers can result in formation of a crosslinked synthetic polymer-naturally occurring polymer matrix upon mixing and/or crosslinking of the synthetic polymer(s). In particular, when the naturally occurring polymer (protein or polysaccharide) also contains nucleophilic groups such as primary amino groups, the electrophilic groups on the second synthetic polymer will react with the primary amino groups on these components, as well as the nucleophilic groups on the first synthetic polymer, to cause these other components to become part of the polymer matrix. For example, lysine-rich proteins such as collagen may be especially reactive with electrophilic groups on synthetic polymers.

In certain embodiments, the naturally occurring protein is polymer may be collagen. As used herein, the term “collagen” or “collagen material” refers to all forms of collagen, including those which have been processed or otherwise modified and is intended to encompass collagen of any type, from any source, including, but not limited to, collagen extracted from tissue or produced recombinantly, collagen analogues, collagen derivatives, modified collagens, and denatured collagens, such as gelatin.

In general, collagen from any source may be included in the compositions of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. U.S. Pat. No. 5,428,022 discloses methods of extracting and purifying collagen from the human placenta. U.S. Pat. No. 5,667,839, discloses methods of producing recombinant human collagen in the milk of transgenic animals, including transgenic cows. Collagen of any type, including, but not limited to, types I, II, III, IV, or any combination thereof, may be used in the compositions of the invention, although type I is generally preferred. Either atelopeptide or telopeptide-containing collagen may be used; however, when collagen from a xenogeneic source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from Inamed Aesthetics (Santa Barbara, Calif.) at collagen concentrations of 35 mg/ml and 65 mg/ml under the trademarks ZYDERM I Collagen and ZYDERM II Collagen, respectively. Glutaraldehyde crosslinked atelopeptide fibrillar collagen is commercially available from Inamed Corporation (Santa Barbara, Calif.) at a collagen concentration of 35 mg/ml under the trademark ZYPLAST Collagen.

Collagens for use in the present invention are generally in aqueous suspension at a concentration between about 20 mg/ml to about 120 mg/ml; preferably, between about 30 mg/ml to about 90 mg/ml.

Because of its tacky consistency, nonfibrillar collagen may be preferred for use in compositions that are intended for use as bioadhesives. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559, issued Aug. 14, 1979, to Miyata et al., which is hereby incorporated by reference in its entirety. Due to its inherent tackiness, methylated collagen is particularly preferred for use in bioadhesive compositions, as disclosed in U.S. application Ser. No. 08/476,825.

Collagens for use in the crosslinked polymer compositions of the present invention may start out in fibrillar form, then be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids (e.g., arginine), inorganic salts (e.g., sodium chloride and potassium chloride), and carbohydrates (e.g., various sugars including sucrose).

In certain embodiments, the polymer may be collagen or a collagen derivative, for example methylated collagen. An example of an in situ forming composition uses pentaerythritol poly(ethylene glycol)ether tetra-sulfhydryl] (4-armed thiol PEG), pentaerythritol poly(ethylene glycol)ether tetra-succinimidyl glutarate] (4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another aspect, the naturally occurring polymer may be a glycosaminoglycan. Glycosaminoglycans, e.g., hyaluronic acid, contain both anionic and cationic functional groups along each polymeric chain, which can form intramolecular and/or intermolecular ionic crosslinks, and are responsible for the thixotropic (or shear thinning) nature of hyaluronic acid.

In certain aspects, the glycosaminoglycan may be derivatized. For example, glycosaminoglycans can be chemically derivatized by, e.g., deacetylation, desulfation, or both in order to contain primary amino groups available for reaction with electrophilic groups on synthetic polymer molecules. Glycosaminoglycans that can be derivatized according to either or both of the aforementioned methods include the following: hyaluronic acid, chondroitin sulfate A, chondroitin sulfate B (dermatan sulfate), chondroitin sulfate C, chitin (can be derivatized to chitosan), keratan sulfate, keratosulfate, and heparin. Derivatization of glycosaminoglycans by deacetylation and/or desulfation and covalent binding of the resulting glycosaminoglycan derivatives with synthetic hydrophilic polymers is described in further detail in commonly assigned, allowed U.S. patent application Ser. No. 08/146,843, filed Nov. 3, 1993.

In general, the collagen is added to the first synthetic polymer, then the collagen and first synthetic polymer are mixed thoroughly to achieve a homogeneous composition. The second synthetic polymer is then added and mixed into the collagen/first synthetic polymer mixture, where it will covalently bind to primary amino groups or thiol groups on the first synthetic polymer and primary amino groups on the collagen, resulting in the formation of a homogeneous crosslinked network. Various deacetylated and/or desulfated glycosaminoglycan derivatives can be incorporated into the composition in a similar manner as that described above for collagen. In addition, the introduction of hydrocolloids such as carboxymethylcellulose may promote tissue adhesion and/or swellability.

Administration of the Crosslinked Synthetic Polymer Compositions

The compositions of the present invention having two synthetic polymers may be administered before, during or after crosslinking of the first and second synthetic polymer. Certain uses, which are discussed in greater detail below, such as tissue augmentation, may require the compositions to be crosslinked before administration, whereas other applications, such as tissue adhesion, require the compositions to be administered before crosslinking has reached “equilibrium.” The point at which crosslinking has reached equilibrium is defined herein as the point at which the composition no longer feels tacky or sticky to the touch.

In order to administer the composition prior to crosslinking, the first synthetic polymer and second synthetic polymer may be contained within separate barrels of a dual-compartment syringe. In this case, the two synthetic polymers do not actually mix until the point at which the two polymers are extruded from the tip of the syringe needle into the patient's tissue. This allows the vast majority of the crosslinking reaction to occur in situ, avoiding the problem of needle blockage which commonly occurs if the two synthetic polymers are mixed too early and crosslinking between the two components is already too advanced prior to delivery from the syringe needle. The use of a dual-compartment syringe, as described above, allows for the use of smaller diameter needles, which is advantageous when performing soft tissue augmentation in delicate facial tissue, such as that surrounding the eyes.

Alternatively, the first synthetic polymer and second synthetic polymer may be mixed according to the methods described above prior to delivery to the tissue site, then injected to the desired tissue site immediately (preferably, within about 60 seconds) following mixing.

In another embodiment of the invention, the first synthetic polymer and second synthetic polymer are mixed, then extruded and allowed to crosslink into a sheet or other solid form. The crosslinked solid is then dehydrated to remove substantially all unbound water. The resulting dried solid may be ground or comminuted into particulates, then suspended in a nonaqueous fluid carrier, including, without limitation, hyaluronic acid, dextran sulfate, dextran, succinylated noncrosslinked collagen, methylated noncrosslinked collagen, glycogen, glycerol, dextrose, maltose, triglycerides of fatty acids (such as corn oil, soybean oil, and sesame oil), and egg yolk phospholipid. The suspension of particulates can be injected through a small-gauge needle to a tissue site. Once inside the tissue, the crosslinked polymer particulates will rehydrate and swell in size at least five-fold.

Hydrophilic Polymer+Plurality of Crosslinkable Components

As mentioned above, the first and/or second synthetic polymers may be combined with a hydrophilic polymer, e.g., collagen or methylated collagen, to form a composition useful in the present invention. In one general embodiment, the compositions useful in the present invention include a hydrophilic polymer in combination with two or more crosslinkable components. This embodiment is described in further detail in this section.

The Hydrophilic Polymer Component:

The hydrophilic polymer component may be a synthetic or naturally occurring hydrophilic polymer. Naturally occurring hydrophilic polymers include, but are not limited to: proteins such as collagen and derivatives thereof, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen (e.g., methylated collagen) and glycosaminoglycans are preferred naturally occurring hydrophilic polymers for use herein.

In general, collagen from any source may be used in the composition of the method; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. See, e.g., U.S. Pat. No. 5,428,022, to Palefsky et al., which discloses methods of extracting and purifying collagen from the human placenta. See also U.S. Pat. No. 5,667,839, to Berg, which discloses methods of producing recombinant human collagen in the milk of transgenic animals, including transgenic cows. Unless otherwise specified, the term “collagen” or “collagen material” as used herein refers to all forms of collagen, including those that have been processed or otherwise modified.

Collagen of any type, including, but not limited to, types I, II, III, IV, or any combination thereof, may be used in the compositions of the invention, although type I is generally preferred. Either atelopeptide or telopeptide-containing collagen may be used; however, when collagen from a source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from McGhan Medical Corporation (Santa Barbara, Calif.) at collagen concentrations of 35 mg/ml and 65 mg/ml under the trademarks ZYDERM® I Collagen and ZYDERM® II Collagen, respectively. Glutaraldehyde-crosslinked atelopeptide fibrillar collagen is commercially available from McGhan Medical Corporation at a collagen concentration of 35 mg/ml under the trademark ZYPLAST®.

Collagens for use in the present invention are generally, although not necessarily, in aqueous suspension at a concentration between about 20 mg/ml to about 120 mg/ml, preferably between about 30 mg/ml to about 90 mg/ml.

Although intact collagen is preferred, denatured collagen, commonly known as gelatin, can also be used in the compositions of the invention. Gelatin may have the added benefit of being degradable faster than collagen.

Because of its greater surface area and greater concentration of reactive groups, nonfibrillar collagen is generally preferred. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen, propylated collagen, ethylated collagen, methylated collagen, and the like, both of which can be prepared according to the methods described in U.S. Pat. No. 4,164,559, to Miyata et al., which is hereby incorporated by reference in its entirety. Due to its inherent tackiness, methylated collagen is particularly preferred, as disclosed in U.S. Pat. No. 5,614,587 to Rhee et al.

Collagens for use in the crosslinkable compositions of the present invention may start out in fibrillar form, then be rendered nonfibrillar by the addition of one or more fiber disassembly agents. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

As fibrillar collagen has less surface area and a lower concentration of reactive groups than nonfibrillar, fibrillar collagen is less preferred. However, as disclosed in U.S. Pat. No. 5,614,587, fibrillar collagen, or mixtures of nonfibrillar and fibrillar collagen, may be preferred for use in compositions intended for long-term persistence in vivo, if optical clarity is not a requirement.

Synthetic hydrophilic polymers may also be used in the present invention. Useful synthetic hydrophilic polymers include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The Crosslinkable Components:

The compositions of the invention also comprise a plurality of crosslinkable components. Each of the crosslinkable components participates in a reaction that results in a crosslinked matrix. Prior to completion of the crosslinking reaction, the crosslinkable components provide the necessary adhesive qualities that enable the methods of the invention.

The crosslinkable components are selected so that crosslinking gives rise to a biocompatible, nonimmunogenic matrix useful in a variety of contexts including adhesion prevention, biologically active agent delivery, tissue augmentation, and other applications. The crosslinkable components of the invention comprise: a component A, which has m nucleophilic groups, wherein m≧2 and a component B, which has n electrophilic groups capable of reaction with the m nucleophilic groups, wherein n≧2 and m+n≧4. An optional third component, optional component C, which has at least one functional group that is either electrophilic and capable of reaction with the nucleophilic groups of component A, or nucleophilic and capable of reaction with the electrophilic groups of component B may also be present. Thus, the total number of functional groups present on components A, B and C, when present, in combination is ≧5; that is, the total functional groups given by m+n+p must be ≧5, where p is the number of functional groups on component C and, as indicated, is ≧1. Each of the components is biocompatible and nonimmunogenic, and at least one component is comprised of a hydrophilic polymer. Also, as will be appreciated, the composition may contain additional crosslinkable components D, E, F, etc., having one or more reactive nucleophilic or electrophilic groups and thereby participate in formation of the crosslinked biomaterial via covalent bonding to other components.

The m nucleophilic groups on component A may all be the same, or, alternatively, A may contain two or more different nucleophilic groups. Similarly, the n electrophilic groups on component B may all be the same, or two or more different electrophilic groups may be present. The functional group(s) on optional component C, if nucleophilic, may or may not be the same as the nucleophilic groups on component A, and, conversely, if electrophilic, the functional group(s) on optional component C may or may not be the same as the electrophilic groups on component B.

Accordingly, the components may be represented by the structural formulae
R¹(-[Q¹]_{q}—X)ₘ (component A), (I)
R²(-[Q²]ᵣ—Y)ₙ (component B), and (II)
R³(-[Q³]ₛ-Fn)ₚ (optional component C), (III)
wherein:

R¹, R² and R³ are independently selected from the group consisting of C₂ to C₁₄ hydrocarbyl, heteroatom-containing C₂ to C₁₄ hydrocarbyl, hydrophilic polymers, and hydrophobic polymers, providing that at least one of R¹, R² and R³ is a hydrophilic polymer, preferably a synthetic hydrophilic polymer;

X represents one of the m nucleophilic groups of component A, and the various X moieties on A may be the same or different;

Y represents one of the n electrophilic groups of component B, and the various Y moieties on A may be the same or different;

Fn represents a functional group on optional component C;

Q¹, Q² and Q³ are linking groups;

m≧2, n≧2, m+n is ≧4, q, and r are independently zero or 1, and when optional component C is present, p≧1, and s is independently zero or 1.

Reactive Groups:

X may be virtually any nucleophilic group, so long as reaction can occur with the electrophilic group Y. Analogously, Y may be virtually any electrophilic group, so long as reaction can take place with X. The only limitation is a practical one, in that reaction between X and Y should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. Ideally, the reactions between X and Y should be complete in under 60 minutes, preferably under 30 minutes. Most preferably, the reaction occurs in about 5 to 15 minutes or less.

Examples of nucleophilic groups suitable as X include, but are not limited to, —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, —C₅H₄N, etc. wherein R⁴ and R⁵ are hydrocarbyl, typically alkyl or monocyclic aryl, preferably alkyl, and most preferably lower alkyl. Organometallic moieties are also useful nucleophilic groups for the purposes of the invention, particularly those that act as carbanion donors. Organometallic nucleophiles are not, however, preferred. Examples of organometallic moieties include: Grignard functionalities —R⁶MgHal wherein R⁶ is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophile. For example, when there are nucleophilic sulfhydryl and hydroxyl groups in the crosslinkable composition, the composition must be admixed with an aqueous base in order to remove a proton and provide an —S⁻ or —O⁻ species to enable reaction with an electrophile. Unless it is desirable for the base to participate in the crosslinking reaction, a nonnucleophilic base is preferred. In some embodiments, the base may be present as a component of a buffer solution. Suitable bases and corresponding crosslinking reactions are described infra.

The selection of electrophilic groups provided within the crosslinkable composition, i.e., on component B, must be made so that reaction is possible with the specific nucleophilic groups. Thus, when the X moieties are amino groups, the Y groups are selected so as to react with amino groups. Analogously, when the X moieties are sulfhydryl moieties, the corresponding electrophilic groups are sulfhydryl-reactive groups, and the like.

By way of example, when X is amino (generally although not necessarily primary amino), the electrophilic groups present on Y are amino reactive groups such as, but not limited to: (1) carboxylic acid esters, including cyclic esters and “activated” esters; (2) acid chloride groups (—CO—Cl); (3) anhydrides (—(CO)—O—(CO)—R); (4) ketones and aldehydes, including α,β-unsaturated aldehydes and ketones such as —CH═CH—CH═O and —CH═CH—C(CH₃)═O; (5) halides; (6) isocyanate (—N═C═O); (7) isothiocyanate (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—CO₂—C═CH₂), methacrylate (—CO₂—C(CH₃)═CH₂)), ethyl acrylate (—CO₂—C(CH₂CH₃)═CH₂), and ethyleneimino (—CH═CH—C═NH). Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Analogously, when X is sulfhydryl, the electrophilic groups present on Y are groups that react with a sulfhydryl moiety. Such reactive groups include those that form thioester linkages upon reaction with a sulfhydryl group, such as those described in PCT Publication No. WO 00/62827 to Wallace et al. As explained in detail therein, such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones. This class of sulfhydryl reactive groups are particularly preferred as the thioether bonds may provide faster crosslinking and longer in vivo stability.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophile such as an epoxide group, an aziridine group, an acyl halide, or an anhydride.

When X is an organometallic nucleophile such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophiles or as electrophiles, depending on the selected reaction partner and/or the reaction conditions. For example, a carboxylic acid group can act as a nucleophile in the presence of a fairly strong base, but generally acts as an electrophile allowing nucleophilic attack at the carbonyl carbon and concomitant replacement of the hydroxyl group with the incoming nucleophile.

The covalent linkages in the crosslinked structure that result upon covalent binding of specific nucleophilic components to specific electrophilic components in the crosslinkable composition include, solely by way of example, the following (the optional linking groups Q¹ and Q² are omitted for clarity):

TABLE 7

| REPRESENTATIVE | | |
|---|---|---|
| NUCLEOPHILIC | | |
| COMPONENT | REPRESENTATIVE | |
| (A, optional | ELECTROPHILIC | |
| component C element | COMPONENT | |
| FN_{NU}) | (B, FN_{EL}) | RESULTING LINKAGE |
| R¹—NH₂ | R²—O—(CO)—O—N(COCH₂) | R¹—NH—(CO)—O—R² |
| | (succinimidyl carbonate | |
| | terminus | |
| R¹—SH | R²—O—(CO)—O—N(COCH₂) | R¹—S—(CO)—O—R² |
| R¹—OH | R²—O—(CO)—O—N(COCH₂) | R¹—O—(CO)—R² |
| R¹—NH₂ | R²—O(CO)—CH═CH₂ (acrylate | R¹—NH—CH₂CH₂—(CO)—O—R² |
| | terminus) | |
| R¹—SH | R²—O—(CO)—CH═CH₂ | R¹—S—CH₂CH₂—(CO)—O—R² |
| R¹—OH | R²—O—(CO)—CH═CH₂ | R¹—O—CH₂CH₂—(CO)—O—R² |
| R¹—NH₂ | R²—O(CO)—(CH₂)₃—CO₂1—N(COCH₂) | R¹—NH—(CO)—(CH₂)₃—(CO)—OR² |
| | (succinimidyl glutarate | |
| | terminus) | |
| R¹—SH | R²—O(CO)—(CH₂)₃—CO₂—N(COCH₂) | R¹—S—(CO)—(CH₂)₃—(CO)—OR² |
| R¹—OH | R²—O(CO)—(CH₂)₃—CO₂—N(COCH₂) | R¹—O—(CO)—(CH₂)₃—(CO)—OR² |
| R¹1'NH₂ | R²—O—CH₂—CO₂—N(COCH₂) | R¹—NH—(CO)—CH₂—OR² |
| | (succinimidyl acetate | |
| | terminus | |
| R¹—SH | R²—O—CH₂—CO₂—N(COCH₂) | R¹—S—(CO)—CH₂—OR² |
| R¹—OH | R²—O—CH₂—CO₂—N(COCH₂) | R¹—O—(CO)—CH₂—OR² |
| R¹—NH₂ | R²—O—NH(CO)—(CH₂)₂—CO₂—N(COCH₂) | R¹—NH—(CO)—(CH₂)₂—(CO)—NH—OR² |
| | (succinimidyl succinamide | |
| | terminus) | |
| R¹SH | R²—O—NH(CO)—(CH₂)₂—CO₂—N(COCH₂) | R¹—S—(CO)—(CH₂)₂—(CO)—NH—OR² |
| R¹—OH | R²—O—NH(CO)—(CH₂)₂—CO₂—N(COCH₂) | R¹—O—(CO)—(CH₂)₂—(CO)—NH—OR² |
| R¹—NH₂ | R²—O—(CH₂)₂—CHO | R¹—NH—(CO)—(CH₂)₂—OR² |
| | (propionaldehyde terminus) | |
| R¹—NH₂ | 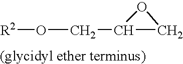 | R¹—NH—CH₂—CH(OH)—CH₂₃—OR² and R¹1'N[CH₂—CH(OH)—CH₂—OR²]₂ |
| R¹—NH₂ | R²—O—(CH₂)₂—N═C═O | R¹1'NH—(CO)—NH—CH₂—OR² |
| | (isocyanate terminus) | |
| R¹—NH₂ | R²—SO₂—CH═CH₂ | R¹—NH—CH₂CH₂—SO₂—R² |
| | (vinyl sulfone terminus) | |
| R¹—SH | R²—SO₂—CH═CH₂ | R¹—S—CH₂CH₂—SO₂—R² |

Linking Groups:

The functional groups X and Y and FN on optional component C may be directly attached to the compound core (R¹, R² or R³ on optional component C, respectively), or they may be indirectly attached through a linking group, with longer linking groups also termed “chain extenders.” In structural formulae (I), (II) and (III), the optional linking groups are represented by Q¹, Q² and Q³, wherein the linking groups are present when q, r and s are equal to 1 (with R, X, Y, Fn, m n and p as defined previously).

Suitable linking groups are well known in the art. See, for example, International Patent Publication No. WO 97/22371. Linking groups are useful to avoid steric hindrance problems that are sometimes associated with the formation of direct linkages between molecules. Linking groups may additionally be used to link several multifunctionally activated compounds together to make larger molecules. In a preferred embodiment, a linking group can be used to alter the degradative properties of the compositions after administration and resultant gel formation. For example, linking groups can be incorporated into components A, B, or optional component C to promote hydrolysis, to discourage hydrolysis, or to provide a site for enzymatic degradation.

Examples of linking groups that provide hydrolyzable sites, include, inter alia: ester linkages; anhydride linkages, such as obtained by incorporation of glutarate and succinate; ortho ester linkages, ortho carbonate linkages such as trimethylene carbonate; amide linkages; phosphoester linkages; α-hydroxy acid linkages, such as may be obtained by incorporation of lactic acid and glycolic acid; lactone-based linkages, such as may be obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, PCT WO 99/07417. Examples of enzymatically degradable linkages include Leu-Gly-Pro-Ala, which is degraded by collagenase; and Gly-Pro-Lys, which is degraded by plasmin.

Linking groups can also enhance or suppress the reactivity of the various nucleophilic and electrophilic groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophile. By contrast, sterically bulky groups in the vicinity of a functional group can be used to diminish reactivity and thus coupling rate as a result of steric hindrance.

By way of example, particular linking groups and corresponding component structure are indicated in Table 8:

TABLE 8

| LINKING GROUP | COMPONENT STRUCTURE |
|---|---|
| —O—(CH₂)ₙ— | Component A: R¹—O—(CH₂)ₙ—X |
| | Component B: R²—O—(CH₂)ₙ—Y |
| | Optional Component C: R³—O—(CH₂)ₙ—Z |
| —S—(CH₂)ₙ— | Component A: R¹—S—(CH₂)ₙ—X |
| | Component B: R²—S—(CH₂)ₙ—Y |
| | Optional Component C: R³—S—(CH₂)ₙ—Z |
| —NH—(CH₂)ₙ— | Component A: R¹—NH—(CH₂)ₙ—X |
| | Component B: R²—NH—(CH₂)ₙ—Y |
| | Optional Component C: R³—NH—(CH₂)ₙ—Z |
| —O—(CO)—NH—(CH₂)ₙ— | Component A: R¹—O—(CO)—NH—(CH₂)ₙ—X |
| | Component B: R²—O—(CO)—NH—(CH₂)ₙ—Y |
| | Optional Component C: R³—O—(CO)—NH—(CH₂)ₙ—Z |
| —NH—(CO)—O—(CH₂)ₙ— | Component A: R¹—NH—(CO)—O—(CH₂)ₙ—X |
| | Component B: R²—NH—(CO)—O—(CH₂)ₙ—Y |
| | Optional Component C: R³—NH—(CO)—O—(CH₂)ₙ—Z |
| —O—(CO)—(CH₂)ₙ— | Component A: R¹—O—(CO)—(CH₂)ₙ—X |
| | Component B: R²—O—(CO)—(CH₂)ₙ—Y |
| | Optional Component C: R³—O—(CO)—(CH₂)ₙ—Z |
| —(CO)—O—(CH₂)ₙ— | Component A: R¹—(CO)—O—(CH₂)ₙ—X |
| | Component B: R²—(CO)—O—(CH₂)ₙ—Y |
| | Optional Component C: R³—(CO)—O—(CH₂)ₙ—Z |
| —O—(CO)—O—(CH₂)ₙ— | Component A: R¹—O—(CO)—O—(CH₂)ₙ—X |
| | Component B: R²—O—(CO)—O—(CH₂)ₙ—Y |
| | Optional Component C: R³—O—(CO)—O—(CH₂)ₙ—Z |
| —O—(CO)—CHR⁷— | Component A: R¹—O—(CO)—CHR⁷—X |
| | Component B: R²—O—(CO)—CHR⁷—Y |
| | Optional Component C: R³—O—(CO)—CHR⁷—Z |
| —O—R⁸—(CO)—NH— | Component A: R¹—O—R⁸—(CO)—NH—X |
| | Component B: R²—O—R⁸—(CO)—NH—Y |
| | Optional Component C: R³—O—R⁸—(CO)—NH—Z |

In the above Table, n is generally in the range of 1 to about 10, R⁷ is generally hydrocarbyl, typically alkyl or aryl, preferably alkyl, and most preferably lower alkyl, and R⁸ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂—).

Other general principles that should be considered with respect to linking groups are as follows: If higher molecular weight components are to be used, they preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

The Component Core:

The “core” of each crosslinkable component is comprised of the molecular structure to which the nucleophilic or electrophilic groups are bound. Using the formulae (I) R¹-[Q¹]_{q}—X)ₘ, for component A, (II) R²(-[Q²]ᵣ—Y)ₙ for component B, and (III)

R³(-[Q³]ₛ-Fn)ₚ for optional component C, the “core” groups are R¹, R² and R³. Each molecular core of the reactive components of the crosslinkable composition is generally selected from synthetic and naturally occurring hydrophilic polymers, hydrophobic polymers, and C₂-C₁₄ hydrocarbyl groups zero to 2 heteroatoms selected from N, O and S, with the proviso that at least one of the crosslinkable components A, B, and optionally C, comprises a molecular core of a synthetic hydrophilic polymer. In a preferred embodiment, at least one of A and B comprises a molecular core of a synthetic hydrophilic polymer.

Hydrophilic Crosslinkable Components

In certain embodiments, the crosslinkable component(s) is (are) hydrophilic polymers. The term “hydrophilic polymer” as used herein refers to a synthetic polymer having an average molecular weight and composition effective to render the polymer “hydrophilic” as defined above. As discussed above, synthetic crosslinkable hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Other suitable synthetic crosslinkable hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Although a variety of different synthetic crosslinkable hydrophilic polymers can be used in the present compositions, as indicated above, preferred synthetic crosslinkable hydrophilic polymers are polyethylene glycol (PEG) and polyglycerol (PG), particularly highly branched polyglycerol. Various forms of PEG are extensively used in the modification of biologically active molecules because PEG lacks toxicity, antigenicity, and immunogenicity (i.e., is biocompatible), can be formulated so as to have a wide range of solubilities, and do not typically interfere with the enzymatic activities and/or conformations of peptides. A particularly preferred synthetic crosslinkable hydrophilic polymer for certain applications is a polyethylene glycol (PEG) having a molecular weight within the range of about 100 to about 100,000 mol. wt., although for highly branched PEG, far higher molecular weight polymers can be employed—up to 1,000,000 or more—providing that biodegradable sites are incorporated ensuring that all degradation products will have a molecular weight of less than about 30,000. For most PEGs, however, the preferred molecular weight is about 1,000 to about 20,000 mol. wt., more preferably within the range of about 7,500 to about 20,000 mol. wt. Most preferably, the polyethylene glycol has a molecular weight of approximately 10,000 mol. wt.

Naturally occurring crosslinkable hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are examples of naturally occurring hydrophilic polymers for use herein, with methylated collagen being a preferred hydrophilic polymer.

Any of the hydrophilic polymers herein must contain, or be activated to contain, functional groups, i.e., nucleophilic or electrophilic groups, which enable crosslinking. Activation of PEG is discussed below; it is to be understood, however, that the following discussion is for purposes of illustration and analogous techniques may be employed with other polymers.

With respect to PEG, first of all, various functionalized polyethylene glycols have been used effectively in fields such as protein modification (see Abuchowski et al., Enzymes as Drugs, John Wiley & Sons: New York, N.Y. (1981) pp. 367-383; and Dreborg et al., Crit. Rev. Therap. Drug Carrier Syst. (1990) 6:315), peptide chemistry (see Mutter et al., The Peptides, Academic: New York, N.Y. 2:285-332; and Zalipsky et al., Int. J. Peptide Protein Res. (1987) 30:740), and the synthesis of polymeric drugs (see Zalipsky et al., Eur. Polym. J. (1983) 19:1177; and Ouchi et al., J. Macromol. Sci. Chem. (1987) A24:1011).

Activated forms of PEG, including multifunctionally activated PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); and Shearwater Polymers, Inc. Catalog, Polyethylene Glycol Derivatives, Huntsville, Ala. (1997-1998).

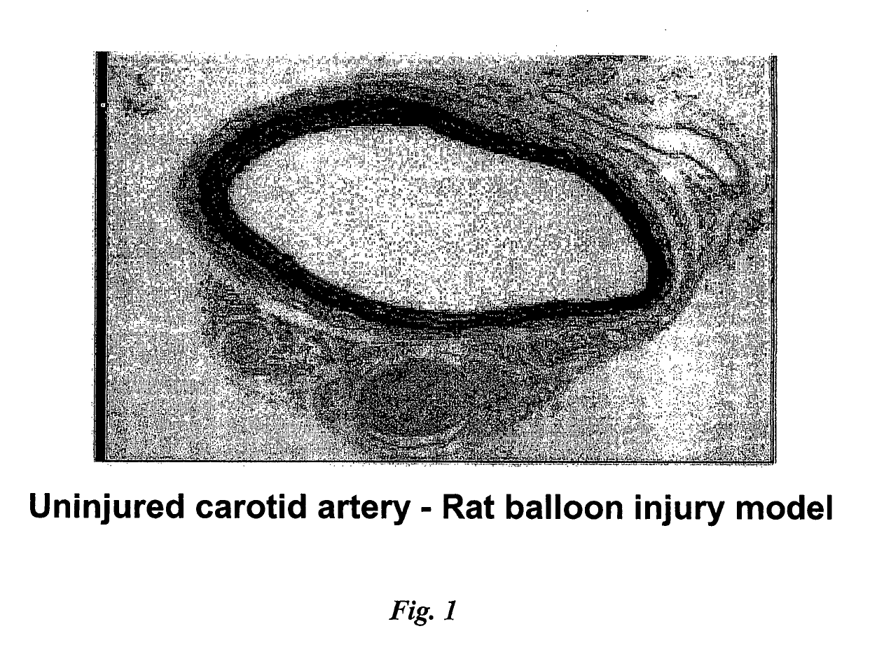

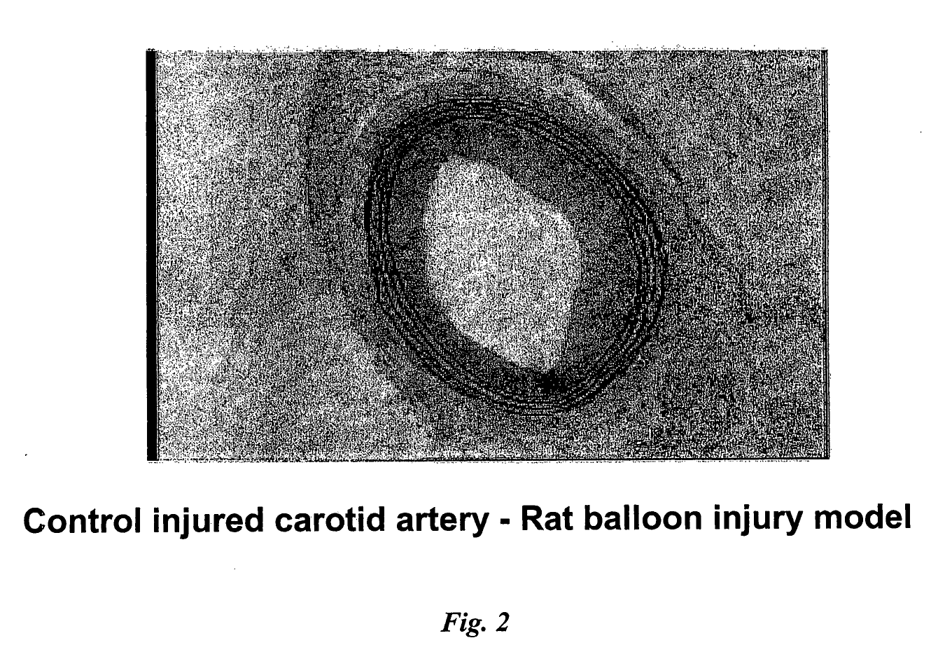

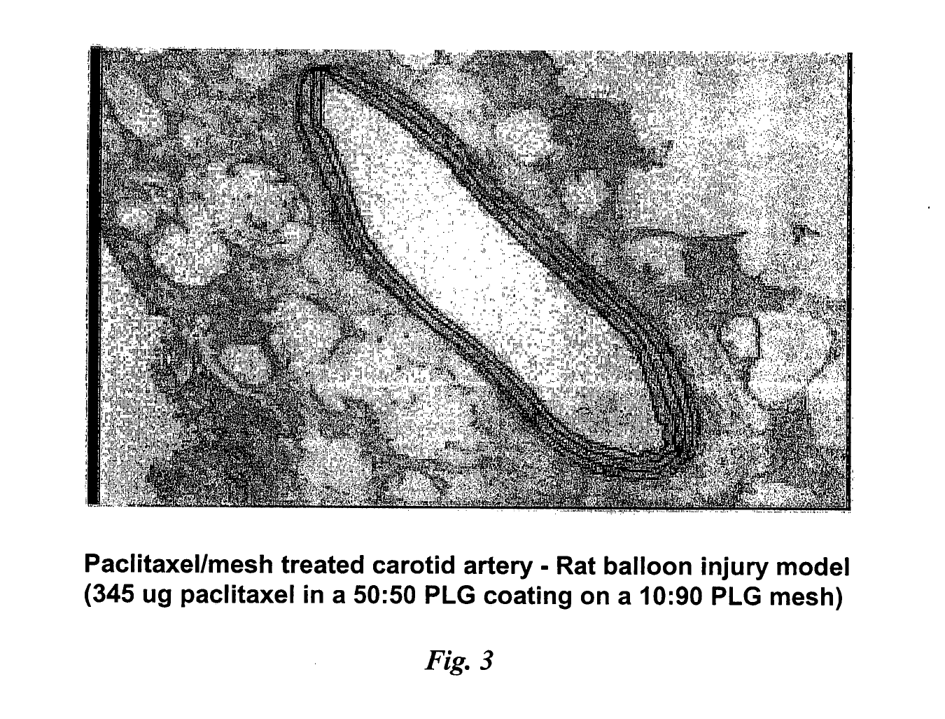

Structures for some specific, tetrafunctionally activated forms of PEG are shown in FIGS. 1 to 10 of U.S. Pat. No. 5,874,500, as are generalized reaction products obtained by reacting the activated PEGs with multi-amino PEGs, i.e., a PEG with two or more primary amino groups. The activated PEGs illustrated have a pentaerythritol (2,2-bis(hydroxymethyl)-1,3-propanediol) core. Such activated PEGs, as will be appreciated by those in the art, are readily prepared by conversion of the exposed hydroxyl groups in the PEGylated polyol (i.e., the terminal hydroxyl groups on the PEG chains) to carboxylic acid groups (typically by reaction with an anhydride in the presence of a nitrogenous base), followed by esterification with N-hydroxysuccinimide, N-hydroxysulfosuccinimide, or the like, to give the polyfunctionally activated PEG.

Hydrophobic Polymers:

The crosslinkable compositions of the invention can also include hydrophobic polymers, although for most uses hydrophilic polymers are preferred. Polylactic acid and polyglycolic acid are examples of two hydrophobic polymers that can be used. With other hydrophobic polymers, only short-chain oligomers should be used, containing at most about 14 carbon atoms, to avoid solubility-related problems during reaction.

Low Molecular Weight Components:

As indicated above, the molecular core of one or more of the crosslinkable components can also be a low molecular weight compound, i.e., a C₂-C₁₄ hydrocarbyl group containing zero to 2 heteroatoms selected from N, O, S and combinations thereof. Such a molecular core can be substituted with nucleophilic groups or with electrophilic groups.

When the low molecular weight molecular core is substituted with primary amino groups, the component may be, for example, ethylenediamine (H₂N—CH₂CH₂—NH₂), tetramethylenediamine (H₂N—(CH₄)—NH₂), pentamethylenediamine (cadaverine) (H₂N—(CH₅)—NH₂), hexamethylenediamine (H₂N—(CH₆)—NH₂), bis(2-aminoethyl)amine (HN—[CH₂CH₂—NH₂]₂), or tris(2-aminoethyl)amine (N—[CH₂CH₂—NH₂]₃).

Low molecular weight diols and polyols include trimethylolpropane, di(trimethylol propane), pentaerythritol, and diglycerol, all of which require activation with a base in order to facilitate their reaction as nucleophiles. Such diols and polyols may also be functionalized to provide di- and poly-carboxylic acids, functional groups that are, as noted earlier herein, also useful as nucleophiles under certain conditions. Polyacids for use in the present compositions include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid), all of which are commercially available and/or readily synthesized using known techniques.

Low molecular weight di- and poly-electrophiles include, for example, disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives. The aforementioned compounds are commercially available from Pierce (Rockford, Ill.). Such di- and poly-electrophiles can also be synthesized from di- and polyacids, for example by reaction with an appropriate molar amount of N-hydroxysuccinimide in the presence of DCC. Polyols such as trimethylolpropane and di(trimethylol propane) can be converted to carboxylic acid form using various known techniques, then further derivatized by reaction with NHS in the presence of DCC to produce trifunctionally and tetrafunctionally activated polymers.

Delivery Systems:

Suitable delivery systems for the homogeneous dry powder composition (containing at least two crosslinkable polymers) and the two buffer solutions may involve a multi-compartment spray device, where one or more compartments contains the powder and one or more compartments contain the buffer solutions needed to provide for the aqueous environment, so that the composition is exposed to the aqueous environment as it leaves the compartment. Many devices that are adapted for delivery of multi-component tissue sealants/hemostatic agents are well known in the art and can also be used in the practice of the present invention. Alternatively, the composition can be delivered using any type of controllable extrusion system, or it can be delivered manually in the form of a dry powder, and exposed to the aqueous environment at the site of administration.

The homogeneous dry powder composition and the two buffer solutions may be conveniently formed under aseptic conditions by placing each of the three ingredients (dry powder, acidic buffer solution and basic buffer solution) into separate syringe barrels. For example, the composition, first buffer solution and second buffer solution can be housed separately in a multiple-compartment syringe system having a multiple barrels, a mixing head, and an exit orifice. The first buffer solution can be added to the barrel housing the composition to dissolve the composition and form a homogeneous solution, which is then extruded into the mixing head. The second buffer solution can be simultaneously extruded into the mixing head. Finally, the resulting composition can then be extruded through the orifice onto a surface.

For example, the syringe barrels holding the dry powder and the basic buffer may be part of a dual-syringe system, e.g., a double barrel syringe as described in U.S. Pat. No. 4,359,049 to Redl et al. In this embodiment, the acid buffer can be added to the syringe barrel that also holds the dry powder, so as to produce the homogeneous solution. In other words, the acid buffer may be added (e.g., injected) into the syringe barrel holding the dry powder to thereby produce a homogeneous solution of the first and second components. This homogeneous solution can then be extruded into a mixing head, while the basic buffer is simultaneously extruded into the mixing head. Within the mixing head, the homogeneous solution and the basic buffer are mixed together to thereby form a reactive mixture. Thereafter, the reactive mixture is extruded through an orifice and onto a surface (e.g., tissue), where a film is formed, which can function as a sealant or a barrier, or the like. The reactive mixture begins forming a three-dimensional matrix immediately upon being formed by the mixing of the homogeneous solution and the basic buffer in the mixing head. Accordingly, the reactive mixture is preferably extruded from the mixing head onto the tissue very quickly after it is formed so that the three-dimensional matrix forms on, and is able to adhere to, the tissue.

Other systems for combining two reactive liquids are well known in the art, and include the systems described in U.S. Pat. Nos. 6,454,786 to Holm et al.; 6,461,325 to Delmotte et al.; 5,585,007 to Antanavich et al.; 5,116,315 to Capozzi et al.; and 4,631,055 to Redl et al.

Storage and Handling:

Because crosslinkable components containing electrophilic groups react with water, the electrophilic component or components are generally stored and used in sterile, dry form to prevent hydrolysis. Processes for preparing synthetic hydrophilic polymers containing multiple electrophilic groups in sterile, dry form are set forth in commonly assigned U.S. Pat. No. 5,643,464 to Rhee et al. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates.

Components containing multiple nucleophilic groups are generally not water-reactive and can therefore be stored either dry or in aqueous solution. If stored as a dry, particulate, solid, the various components of the crosslinkable composition may be blended and stored in a single container. Admixture of all components with water, saline, or other aqueous media should not occur until immediately prior to use.

In an alternative embodiment, the crosslinking components can be mixed together in a single aqueous medium in which they are both unreactive, i.e., such as in a low pH buffer. Thereafter, they can be sprayed onto the targeted tissue site along with a high pH buffer, after which they will rapidly react and form a gel.

Suitable liquid media for storage of crosslinkable compositions include aqueous buffer solutions such as monobasic sodium phosphate/dibasic sodium phosphate, sodium carbonate/sodium bicarbonate, glutamate or acetate, at a concentration of 0.5 to 300 mM. In general, a sulfhydryl-reactive component such as PEG substituted with maleimido groups or succinimidyl esters is prepared in water or a dilute buffer, with a pH of between around 5 to 6. Buffers with pKs between about 8 and 10.5 for preparing a polysulfhydryl component such as sulfhydryl-PEG are useful to achieve fast gelation time of compositions containing mixtures of sulfhydryl-PEG and SG-PEG. These include carbonate, borate and AMPSO (3-[(1,1-dimethyl-2-hydroxyethyl)amino]2-hydroxy-propane-sulfonic acid). In contrast, using a combination of maleimidyl PEG and sulfhydryl-PEG, a pH of around 5 to 9 is preferred for the liquid medium used to prepare the sulfhydryl PEG.

Collagen+Fibrinogen and/or Thrombin (e.g., Costasis)

In yet another aspect, the polymer composition may include collagen in combination with fibrinogen and/or thrombin. (See, e.g., U.S. Pat. Nos. 5,290,552; 6,096,309; and 5,997,811). For example, an aqueous composition may include a fibrinogen and FXIII, particularly plasma, collagen in an amount sufficient to thicken the composition, thrombin in an amount sufficient to catalyze polymerization of fibrinogen present in the composition, and Ca²⁺ and, optionally, an antifibrinolytic agent in amount sufficient to retard degradation of the resulting adhesive clot. The composition may be formulated as a two-part composition that may be mixed together just prior to use, in which fibrinogen/FXIII and collagen constitute the first component, and thrombin together with an antifibrinolytic agent, and Ca²⁺ constitute the second component.

Plasma, which provides a source of fibrinogen, may be obtained from the patient for which the composition is to be delivered. The plasma can be used “as is” after standard preparation which includes centrifuging out cellular components of blood. Alternatively, the plasma can be further processed to concentrate the fibrinogen to prepare a plasma cryoprecipitate. The plasma cryoprecipitate can be prepared by freezing the plasma for at least about an hour at about −20° C., and then storing the frozen plasma overnight at about 4° C. to slowly thaw. The thawed plasma is centrifuged and the plasma cryoprecipitate is harvested by removing approximately four-fifths of the plasma to provide a cryoprecipitate comprising the remaining one-fifth of the plasma. Other fibrinogen/FXIII preparations may be used, such as cryoprecipitate, patient autologous fibrin sealant, fibrinogen analogs or other single donor or commercial fibrin sealant materials. Approximately 0.5 ml to about 1.0 ml of either the plasma or the plasma-cryoprecipitate provides about 1 to 2 ml of adhesive composition which is sufficient for use in middle ear surgery. Other plasma proteins (e.g., albumin, plasminogen, von Willebrands factor, Factor VIII, etc.) may or may not be present in the fibrinogen/FXII separation due to wide variations in the formulations and methods to derive them.

Collagen, preferably hypoallergenic collagen, is present in the composition in an amount sufficient to thicken the composition and augment the cohesive properties of the preparation. The collagen may be atelopeptide collagen or telopeptide collagen, e.g., native collagen. In addition to thickening the composition, the collagen augments the fibrin by acting as a macromolecular lattice work or scaffold to which the fibrin network adsorbs. This gives more strength and durability to the resulting glue clot with a relatively low concentration of fibrinogen in comparison to the various concentrated autogenous fibrinogen glue formulations (i.e., AFGs).

The form of collagen which is employed may be described as at least “near native” in its structural characteristics. It may be further characterized as resulting in insoluble fibers at a pH above 5; unless crosslinked or as part of a complex composition, e.g., bone, it will generally consist of a minor amount by weight of fibers with diameters greater than 50 nm, usually from about 1 to 25 volume % and there will be substantially little, if any, change in the helical structure of the fibrils. In addition, the collagen composition must be able to enhance gelation in the surgical adhesion composition.

A number of commercially available collagen preparations may be used. ZYDERM Collagen Implant (ZCI) has a fibrillar diameter distribution consisting of 5 to 10 nm diameter fibers at 90% volume content and the remaining 10% with greater than about 50 nm diameter fibers. ZCI is available as a fibrillar slurry and solution in phosphate buffered isotonic saline, pH 7.2, and is injectable with fine gauge needles. As distinct from ZCI, cross-linked collagen available as ZYPLAST may be employed. ZYPLAST is essentially an exogenously crosslinked (glutaraldehyde) version of ZCI. The material has a somewhat higher content of greater than about 50 nm diameter fibrils and remains insoluble over a wide pH range. Crosslinking has the effect of mimicking in vivo endogenous crosslinking found in many tissues.

Thrombin acts as a catalyst for fibrinogen to provide fibrin, an insoluble polymer and is present in the composition in an amount sufficient to catalyze polymerization of fibrinogen present in the patient plasma. Thrombin also activates FXIII, a plasma protein that catalyzes covalent crosslinks in fibrin, rendering the resultant clot insoluble. Usually the thrombin is present in the adhesive composition in concentration of from about 0.01 to about 1000 or greater NIH units (NIHu) of activity, usually about i to about 500 NIHu, most usually about 200 to about 500 NIHu. The thrombin can be from a variety of host animal sources, conveniently bovine. Thrombin is commercially available from a variety of sources including Parke-Davis, usually lyophilized with buffer salts and stabilizers in vials which provide thrombin activity ranging from about 1000 NIHu to 10,000 NIHu. The thrombin is usually prepared by reconstituting the powder by the addition of either sterile distilled water or isotonic saline. Alternately, thrombin analogs or reptile-sourced coagulants may be used.

The composition may additionally comprise an effective amount of an antifibrinolytic agent to enhance the integrity of the glue clot as the healing processes occur. A number of antifibrinolytic agents are well known and include aprotinin, C1-esterase inhibitor and ε-amino-n-caproic acid (EACA). ε-amino-n-caproic acid, the only antifibrinolytic agent approved by the FDA, is effective at a concentration of from about 5 mg/ml to about 40 mg/ml of the final adhesive composition, more usually from about 20 to about 30 mg/ml. EACA is commercially available as a solution having a concentration of about 250 mg/ml. Conveniently, the commercial solution is diluted with distilled water to provide a solution of the desired concentration. That solution is desirably used to reconstitute lyophilized thrombin to the desired thrombin concentration.

Other examples of in situ forming materials based on the crosslinking of proteins are described, e.g., in U.S. Pat. Nos. RE 38158; 4,839,345; 5,514,379, 5,583,114; 6,458,147; 6,371,975; 5,290,552; 6,096,309; U.S. Patent Application Publication Nos. 2002/0161399; 2001/0018598 and PCT Publication Nos. WO 03/090683; WO 01/45761; WO 99/66964 and WO 96/03159).

Self-Reactive Compounds

In certain embodiments, the drug combination, or individual component(s) thereof, is released from a crosslinked matrix formed, at least in part, from a self-reactive compound. As used herein, a self-reactive compound comprises a core substituted with a minimum of three reactive groups. The reactive groups may be directed attached to the core of the compound, or the reactive groups may be indirectly attached to the compound's core, e.g., the reactive groups are joined to the core through one or more linking groups.

Each of the three reactive groups that are necessarily present in a self-reactive compound can undergo a bond-forming reaction with at least one of the remaining two reactive groups. For clarity it is mentioned that when these compounds react to form a crosslinked matrix, it will most often happen that reactive groups on one compound will reactive with reactive groups on another compound. That is, the term “self-reactive” is not intended to mean that each self-reactive compound necessarily reacts with itself, but rather that when a plurality of identical self-reactive compounds are in combination and undergo a crosslinking reaction, then these compounds will react with one another to form the matrix. The compounds are “self-reactive” in the sense that they can react with other compounds having the identical chemical structure as themselves.

The self-reactive compound comprises at least four components: a core and three reactive groups. In one embodiment, the self-reactive compound can be characterized by the formula (I), where R is the core, the reactive groups are represented by X¹, X² and X³, and a linker (L) is optionally present between the core and a functional group.

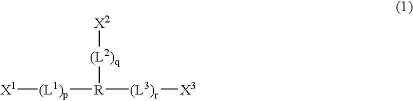

The core R is a polyvalent moiety having attachment to at least three groups (i.e., it is at least trivalent) and may be, or may contain, for example, a hydrophilic polymer, a hydrophobic polymer, an amphiphilic polymer, a C₂₋₁₄ hydrocarbyl, or a C₂₋₁₄ hydrocarbyl which is heteroatom-containing. The linking groups L¹, L², and L³ may be the same or different. The designators p, q and r are either 0 (when no linker is present) or 1 (when a linker is present). The reactive groups X¹, X² and X³ may be the same or different. Each of these reactive groups reacts with at least one other reactive group to form a three-dimensional matrix. Therefore X¹ can react with X² and/or X³, X² can react with X¹ and/or X³, X³ can react with X¹ and/or X² and so forth. A trivalent core will be directly or indirectly bonded to three functional groups, a tetravalent core will be directly or indirectly bonded to four functional groups, etc.

Each side chain typically has one reactive group. However, the invention also encompasses self-reactive compounds where the side chains contain more than one reactive group. Thus, in another embodiment of the invention, the self-reactive compound has the formula (II):
[X₁-(L⁴)ₐ-Y′-(L⁵)_{b}]_{c}-R′
where: a and b are integers from 0-1; c is an integer from 3-12; R′ is selected from hydrophilic polymers, hydrophobic polymers, amphiphilic polymers, C₂₋₁₄ hydrocarbyls, and heteroatom-containing C₂₋₁₄ hydrocarbyls; X′ and Y′ are reactive groups and can be the same or different; and L⁴ and L⁵ are linking groups. Each reactive group inter-reacts with the other reactive group to form a three-dimensional matrix. The compound is essentially non-reactive in an initial environment but is rendered reactive upon exposure to a modification in the initial environment that provides a modified environment such that a plurality of the self-reactive compounds inter-react in the modified environment to form a three-dimensional matrix. In one preferred embodiment, R is a hydrophilic polymer. In another preferred embodiment, X′ is a nucleophilic group and Y′ is an electrophilic group.

The following self-reactive compound is one example of a compound of formula (II): 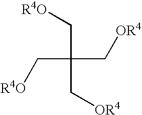
where R⁴ has the formula:

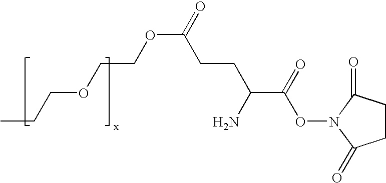

Thus, in formula (II), a and b are 1; c is 4; the core R′ is the hydrophilic polymer, tetrafunctionally activated polyethylene glycol, (C(CH₂—O—)₄; X′ is the electrophilic reactive group, succinimidyl; Y′ is the nucleophilic reactive group —CH—NH₂; L⁴ is —C(O)—O—; and L⁵ is —(CH₂—CH₂—O—CH₂)ₓ—CH₂—O—C(O)—(CH₂)₂—.

The self-reactive compounds of the invention are readily synthesized by techniques that are well known in the art. An exemplary synthesis is set forth below:

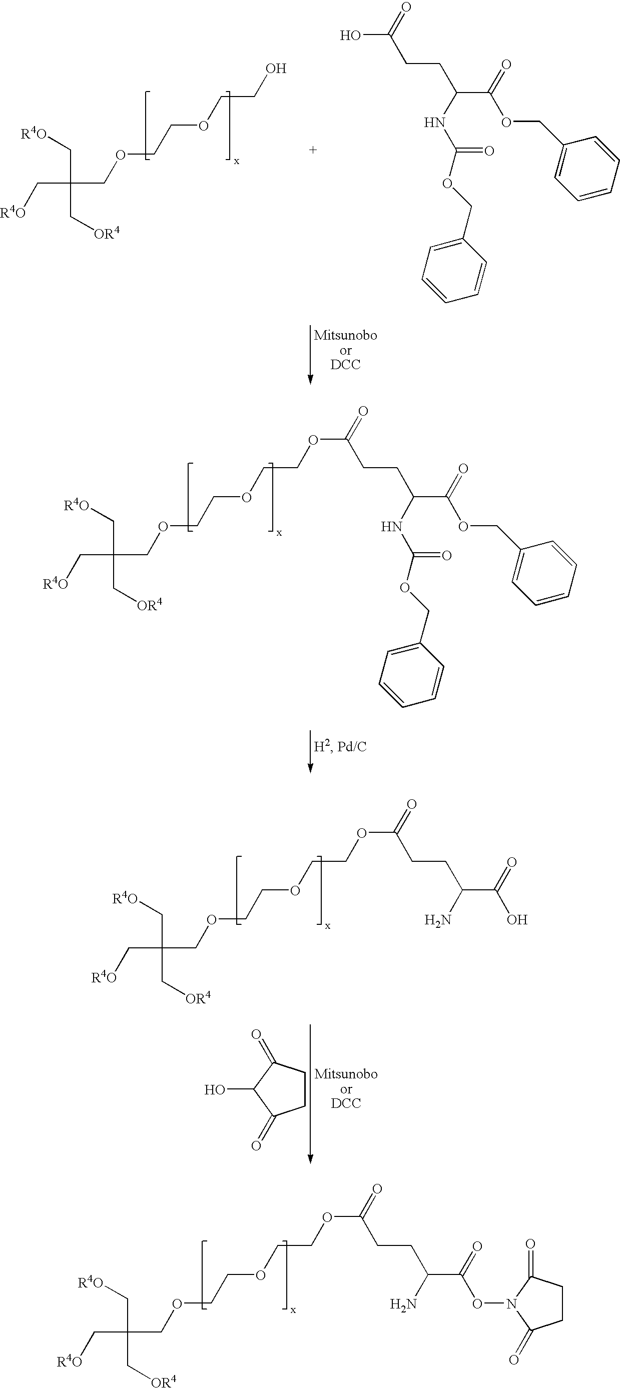

The reactive groups are selected so that the compound is essentially non-reactive in an initial environment. Upon exposure to a specific modification in the initial environment, providing a modified environment, the compound is rendered reactive and a plurality of self-reactive compounds are then able to inter-react in the modified environment to form a three-dimensional matrix. Examples of modification in the initial environment are detailed below, but include the addition of an aqueous medium, a change in pH, exposure to ultraviolet radiation, a change in temperature, or contact with a redox initiator.

The core and reactive groups can also be selected so as to provide a compound that has one of more of the following features: are biocompatible, are non-immunogenic, and do not leave any toxic, inflammatory or immunogenic reaction products at the site of administration. Similarly, the core and reactive groups can also be selected so as to provide a resulting matrix that has one or more of these features.

In one embodiment of the invention, substantially immediately or immediately upon exposure to the modified environment, the self-reactive compounds inter-react form a three-dimensional matrix. The term “substantially immediately” is intended to mean within less than five minutes, preferably within less than two minutes, and the term “immediately” is intended to mean within less than one minute, preferably within less than 30 seconds.

In one embodiment, the self-reactive compound and resulting matrix are not subject to enzymatic cleavage by matrix metalloproteinases such as collagenase, and are therefore not readily degradable in vivo. Further, the self-reactive compound may be readily tailored, in terms of the selection and quantity of each component, to enhance certain properties, e.g., compression strength, swellability, tack, hydrophilicity, optical clarity, and the like.

In one preferred embodiment, R is a hydrophilic polymer. In another preferred embodiment, X is a nucleophilic group, Y is an electrophilic group and Z is either an electrophilic or a nucleophilic group. Additional embodiments are detailed below.

A higher degree of inter-reaction, e.g., crosslinking, may be useful when a less swellable matrix is desired or increased compressive strength is desired. In those embodiments, it may be desirable to have n be an integer from 2-12. In addition, when a plurality of self-reactive compounds are utilized, the compounds may be the same or different.

Reactive Groups

Prior to use, the self-reactive compound is stored in an initial environment that insures that the compound remain essentially non-reactive until use. Upon modification of this environment, the compound is rendered reactive and a plurality of compounds will then inter-react to form the desired matrix. The initial environment, as well as the modified environment, is thus determined by the nature of the reactive groups involved.

The number of reactive groups can be the same or different. However, in one embodiment of the invention, the number of reactive groups is approximately equal. As used in this context, the term “approximately” refers to a 2:1 to 1:2 ratio of moles of one reactive group to moles of a different reactive groups. A 1:1:1 molar ratio of reactive groups is generally preferred.

In general, the concentration of the self-reactive compounds in the modified environment, when liquid in nature, will be in the range of about 1 to 50 wt %, generally about 2 to 40 wt %. The preferred concentration of the compound in the liquid will depend on a number of factors, including the type of compound (i.e., type of molecular core and reactive groups), its molecular weight, and the end use of the resulting three-dimensional matrix. For example, use of higher concentrations of the compounds, or using highly functionalized compounds, will result in the formation of a more tightly crosslinked network, producing a stiffer, more robust gel. As such, compositions intended for use in tissue augmentation will generally employ concentrations of self-reactive compounds that fall toward the higher end of the preferred concentration range. Compositions intended for use as bioadhesives or in adhesion prevention do not need to be as firm and may therefore contain lower concentrations of the self-reactive compounds.

Electrophilic and Nucleophilic Reactive Groups

In one embodiment of the invention, the reactive groups are electrophilic and nucleophilic groups, which undergo a nucleophilic substitution reaction, a nucleophilic addition reaction, or both. The term “electrophilic” refers to a reactive group that is susceptible to nucleophilic attack, i.e., susceptible to reaction with an incoming nucleophilic group. Electrophilic groups herein are positively charged or electron-deficient, typically electron-deficient. The term “nucleophilic” refers to a reactive group that is electron rich, has an unshared pair of electrons acting as a reactive site, and reacts with a positively charged or electron-deficient site. For such reactive groups, the modification in the initial environment comprises the addition of an aqueous medium and/or a change in pH.

In one embodiment of the invention, X1 (also referred to herein as X) can be a nucleophilic group and X2 (also referred to herein as Y) can be an electrophilic group or vice versa, and X3 (also referred to herein as Z) can be either an electrophilic or a nucleophilic group.

X may be virtually any nucleophilic group, so long as reaction can occur with the electrophilic group Y and also with Z, when Z is electrophilic (Z_{EL}). Analogously, Y may be virtually any electrophilic group, so long as reaction can take place with X and also with Z when Z is nucleophilic (Z_{NU}). The only limitation is a practical one, in that reaction between X and Y, and X and Z_{EL}, or Y and Z_{NU} should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. In one embodiment, the reactions between X and Y, and between either X and Z_{EL} or Y and Z_{NU}, are complete in under 60 minutes, preferably under 30 minutes. Most preferably, the reaction occurs in about 5 to 15 minutes or less.

Examples of nucleophilic groups suitable as X or Fn_{NU} include, but are not limited to: —NH₂, —NHR¹, —N(R¹)₂, —SH, —OH, —COOH, —C₆H₄—OH, —H, —PH₂, —PHR¹, —P(R¹)₂, —NH—NH₂, —CO—NH—NH₂, —C₅H₄N, etc. wherein R¹ is a hydrocarbyl group and each R¹ may be the same or different. R¹ is typically alkyl or monocyclic aryl, preferably alkyl, and most preferably lower alkyl. Organometallic moieties are also useful nucleophilic groups for the purposes of the invention, particularly those that act as carbanion donors. Examples of organometallic moieties include: Grignard functionalities —R²MgHal wherein R² is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophilic group. For example, when there are nucleophilic sulfhydryl and hydroxyl groups in the self-reactive compound, the compound must be admixed with an aqueous base in order to remove a proton and provide an —S⁻ or —O⁻ species to enable reaction with the electrophilic group. Unless it is desirable for the base to participate in the reaction, a non-nucleophilic base is preferred. In some embodiments, the base may be present as a component of a buffer solution. Suitable bases and corresponding crosslinking reactions are described herein.

The selection of electrophilic groups provided on the self-reactive compound, must be made so that reaction is possible with the specific nucleophilic groups. Thus, when the X reactive groups are amino groups, the Y and any Z_{EL} groups are selected so as to react with amino groups. Analogously, when the X reactive groups are sulfhydryl moieties, the corresponding electrophilic groups are sulfhydryl-reactive groups, and the like. In general, examples of electrophilic groups suitable as Y or Z_{EL} include, but are not limited to, —CO—Cl, —(CO)—O—(CO)—R (where R is an alkyl group), —CH═CH—CH═O and —CH═CH—C(CH₃)═O, halo, —N═C═O, —N═C═S, —SO₂CH═CH₂, —O(CO)—C═CH₂, —O(CO)—C(CH₃)═CH₂, —S—S—(C₅H₄N), —O(CO)—C(CH₂CH₃)═CH₂, —CH═CH—C═NH, —COOH, —(CO)O—N(COCH₂)₂, —CHO, —(CO)O—N(COCH₂)₂—S(O)₂OH, and —N(COCH)₂.

When X is amino (generally although not necessarily primary amino), the electrophilic groups present on Y and Z_{EL} are amine-reactive groups. Exemplary amine-reactive groups include, by way of example and not limitation, the following groups, or radicals thereof: (1) carboxylic acid esters, including cyclic esters and “activated” esters; (2) acid chloride groups (—CO—Cl); (3) anhydrides (—(CO)—O—(CO)—R, where R is an alkyl group); (4) ketones and aldehydes, including α,β-unsaturated aldehydes and ketones such as —CH═CH—CH═O and —CH═CH—C(CH₃)═O; (5) halo groups; (6) isocyanate group (—N═C═O); (7) thioisocyanato group (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—O(CO)—C═CH₂), methacrylate (—O(CO)—C(CH₃)═CH₂), ethyl acrylate (—O(CO)—C(CH₂CH₃)═CH₂), and ethyleneimino (—CH═CH—C═NH).

In one embodiment the amine-reactive groups contain an electrophilically reactive carbonyl group susceptible to nucleophilic attack by a primary or secondary amine, for example the carboxylic acid esters and aldehydes noted above, as well as carboxyl groups (—COOH).

Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Accordingly, in one embodiment, the amine-reactive groups are selected from succinimidyl ester (—O(CO)—N(COCH₂)₂), sulfosuccinimidyl ester (—O(CO)—N(COCH₂)₂—S(O)₂OH), maleimido (—N(COCH)₂), epoxy, isocyanato, thioisocyanato, and ethenesulfonyl.

Analogously, when X is sulfhydryl, the electrophilic groups present on Y and Z_{EL} are groups that react with a sulfhydryl moiety. Such reactive groups include those that form thioester linkages upon reaction with a sulfhydryl group, such as those described in WO 00/62827 to Wallace et al. As explained in detail therein, sulfhydryl reactive groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophilic group such as an epoxide group, an aziridine group, an acyl halide, an anhydride, and so forth.

When X is an organometallic nucleophilic group such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophilic or as electrophilic groups, depending on the selected reaction partner and/or the reaction conditions. For example, a carboxylic acid group can act as a nucleophilic group in the presence of a fairly strong base, but generally acts as an electrophilic group allowing nucleophilic attack at the carbonyl carbon and concomitant replacement of the hydroxyl group with the incoming nucleophilic group.

These, as well as other embodiments are illustrated below, where the covalent linkages in the matrix that result upon covalent binding of specific nucleophilic reactive groups to specific electrophilic reactive groups on the self-reactive compound include, solely by way of example, the following Table 9:

TABLE 9

| Representative | | |
|---|---|---|
| Nucleophilic | Representative Electrophilic Group | |
| Group (X, Z_{NU}) | (Y, Z_{EL}) | Resulting Linkage |
| —NH₂ | —O—(CO)—O—N(COCH₂)₂ | —NH—(CO)—O— |
| | succinimidyl carbonate terminus | |
| —SH | —O—(CO)—O—N(COCH₂)₂ | —S—(CO)—O— |
| —OH | —O—(CO)—O—N(COCH₂)₂ | —O—(CO)— |
| —NH₂ | —O(CO)—CH═CH₂ | —NH—CH₂CH₂—(CO)—O— |
| | acrylate terminus | |
| —SH | —O—(CO)—CH═CH₂ | —S—CH₂CH₂—(CO)—O— |
| —OH | —O—(CO)—CH═CH₂ | —O—CH₂CH₂—(CO)—O— |
| —NH₂ | —O(CO)—(CH₂)₃—CO₂—N(COCH₂)₂ | —NH—(CO)—(CH₂)₃—(CO)—O— |
| | succinimidyl glutarate terminus | |
| —SH | —O(CO)—(CH₂)₃—CO₂—N(COCH₂)₂ | —S+113 (CO)—(CH₂)₃—(CO)—O— |
| —OH | —O(CO)—(CH₂)₃—CO₂—N(COCH₂)₂ | —O—(CO)—(CH₂)₃—(CO)—O— |
| —NH₂ | —O—CH₂—CO₂—N(COCH₂)₂ | —NH—(CO)—CH₂—O— |
| | succinimidyl acetate terminus | |
| —SH | —O—CH₂—CO₂—N(COCH₂)₂ | —S—(CO)—CH₂—O— |
| —OH | —O—CH₂—CO₂—N(COCH₂)₂ | —O—(CO)—CH₂—O— |
| —NH₂ | —O—NH(CO)—(CH₂)₂—CO₂—N(COCH₂)₂ | —NH—(CO)—(CH₂)₂—(CO)—NH—O— |
| | succinimidyl succinamide terminus | |
| —SH | —O—NH(CO)—(CH₂)₂—CO₂—N(COCH₂)₂ | —S—(CO)—(CH₂)₂—(CO)—NH—O— |
| —OH | —O—NH(CO)—(CH₂)₂—CO₂—N(COCH₂)₂ | —O—(CO)—(CH₂)₂—(CO)—NH—O— |
| —NH₂ | —O—(CH₂)₂—CHO | —NH—(CO)—(CH₂)₂—O— |
| | propionaldehyde terminus | |
| —NH₂ | 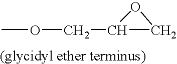 | —NH—CH₂—CH(OH)—CH₂—O—and —N[CH₂—CH(OH)—CH₂—O—]₂ |
| —NH₂ | —O—(CH₂)₂—N═C═O | —NH—(CO)—NH—CH₂—O— |
| | (isocyanate terminus) | |
| —NH₂ | —SO₂—CH═CH₂ | —NH—CH₂CH₂—SO₂— |
| | vinyl sulfone terminus | |
| —S | —SO₂—CH═CH₂ | —S—CH₂CH₂—SO₂— |

For self-reactive compounds containing electrophilic and nucleophilic reactive groups, the initial environment typically can be dry and sterile. Since electrophilic groups react with water, storage in sterile, dry form will prevent hydrolysis. The dry synthetic polymer may be compression molded into a thin sheet or membrane, which can then be sterilized using gamma or e-beam irradiation. The resulting dry membrane or sheet can be cut to the desired size or chopped into smaller size particulates. The modification of a dry initial environment will typically comprise the addition of an aqueous medium.

In one embodiment, the initial environment can be an aqueous medium such as in a low pH buffer, i.e., having a pH less than about 6.0, in which both electrophilic and nucleophilic groups are non-reactive. Suitable liquid media for storage of such compounds include aqueous buffer solutions such as monobasic sodium phosphate/dibasic sodium phosphate, sodium carbonate/sodium bicarbonate, glutamate or acetate, at a concentration of 0.5 to 300 mM. Modification of an initial low pH aqueous environment will typically comprise increasing the pH to at least pH 7.0, more preferably increasing the pH to at least pH9.5.

In another embodiment the modification of a dry initial environment comprises dissolving the self-reactive compound in a first buffer solution having a pH within the range of about 1.0 to 5.5 to form a homogeneous solution, and (ii) adding a second buffer solution having a pH within the range of about 6.0 to 11.0 to the homogeneous solution. The buffer solutions are aqueous and can be any pharmaceutically acceptable basic or acid composition. The term “buffer” is used in a general sense to refer to an acidic or basic aqueous solution, where the solution may or may not be functioning to provide a buffering effect (i.e., resistance to change in pH upon addition of acid or base) in the compositions of the present invention. For example, the self-reactive compound can be in the form of a homogeneous dry powder. This powder is then combined with a buffer solution having a pH within the range of about 1.0 to 5.5 to form a homogeneous acidic aqueous solution, and this solution is then combined with a buffer solution having a pH within the range of about 6.0 to 11.0 to form a reactive solution. For example, 0.375 grams of the dry powder can be combined with 0.75 grams of the acid buffer to provide, after mixing, a homogeneous solution, where this solution is combined with 1.1 grams of the basic buffer to provide a reactive mixture that substantially immediately forms a three-dimensional matrix.

Acidic buffer solutions having a pH within the range of about 1.0 to 5.5, include by way of illustration and not limitation, solutions of: citric acid, hydrochloric acid, phosphoric acid, sulfuric acid, AMPSO (3-[(1,1-dimethyl-2-hydroxyethyl)amino]2-hydroxy-propane-sulfonic acid), acetic acid, lactic acid, and combinations thereof. In a preferred embodiment, the acidic buffer solution is a solution of citric acid, hydrochloric acid, phosphoric acid, sulfuric acid, and combinations thereof. Regardless of the precise acidifying agent, the acidic buffer preferably has a pH such that it retards the reactivity of the nucleophilic groups on the core. For example, a pH of 2.1 is generally sufficient to retard the nucleophilicity of thiol groups. A lower pH is typically preferred when the core contains amine groups as the nucleophilic groups. In general, the acidic buffer is an acidic solution that, when contacted with nucleophilic groups, renders those nucleophilic groups relatively non-nucleophilic.

An exemplary acidic buffer is a solution of hydrochloric acid, having a concentration of about 6.3 mM and a pH in the range of 2.1 to 2.3. This buffer may be prepared by combining concentrated hydrochloric acid with water, i.e., by diluting concentrated hydrochloric acid with water. Similarly, this buffer A may also be conveniently prepared by diluting 1.23 grams of concentrated hydrochloric acid to a volume of 2 liters, or diluting 1.84 grams of concentrated hydrochloric acid to a volume to 3 liters, or diluting 2.45 grams of concentrated hydrochloric acid to a volume of 4 liters, or diluting 3.07 grams concentrated hydrochloric acid to a volume of 5 liters, or diluting 3.68 grams of concentrated hydrochloric acid to a volume to 6 liters. For safety reasons, the concentrated acid is preferably added to water.

Basic buffer solutions having a pH within the range of about 6.0 to 11.0, include by way of illustration and not limitation, solutions of: glutamate, acetate, carbonate and carbonate salts (e.g., sodium carbonate, sodium carbonate monohydrate and sodium bicarbonate), borate, phosphate and phosphate salts (e.g., monobasic sodium phosphate monohydrate and dibasic sodium phosphate), and combinations thereof. In a preferred embodiment, the basic buffer solution is a solution of carbonate salts, phosphate salts, and combinations thereof.

In general, the basic buffer is an aqueous solution that neutralizes the effect of the acidic buffer, when it is added to the homogeneous solution of the compound and first buffer, so that the nucleophilic groups on the core regain their nucleophilic character (that has been masked by the action of the acidic buffer), thus allowing the nucleophilic groups to inter-react with the electrophilic groups on the core.

An exemplary basic buffer is an aqueous solution of carbonate and phosphate salts. This buffer may be prepared by combining a base solution with a salt solution. The salt solution may be prepared by combining 34.7 g of monobasic sodium phosphate monohydrate, 49.3 g of sodium carbonate monohydrate, and sufficient water to provide a solution volume of 2 liter. Similarly, a 6 liter solution may be prepared by combining 104.0 g of monobasic sodium phosphate monohydrate, 147.94 g of sodium carbonate monohydrate, and sufficient water to provide 6 liter of the salt solution. The basic buffer may be prepared by combining 7.2 g of sodium hydroxide with 180.0 g of water. The basic buffer is typically prepared by adding the base solution as needed to the salt solution, ultimately to provide a mixture having the desired pH, e.g., a pH of 9.65 to 9.75.

In general, the basic species present in the basic buffer should be sufficiently basic to neutralize the acidity provided by the acidic buffer, but should not be so nucleophilic itself that it will react substantially with the electrophilic groups on the core. For this reason, relatively “soft” bases such as carbonate and phosphate are preferred in this embodiment of the invention.

To illustrate the preparation of a three-dimensional matrix of the present invention, one may combine an admixture of the self-reactive compound with a first, acidic, buffer (e.g., an acid solution, e.g., a dilute hydrochloric acid solution) to form a homogeneous solution. This homogeneous solution is mixed with a second, basic, buffer (e.g., a basic solution, e.g., an aqueous solution containing phosphate and carbonate salts) whereupon the reactive groups on the core of the self-reactive compound substantially immediately inter-react with one another to form a three-dimensional matrix.

Redox Reactive Groups

In one embodiment of the invention, the reactive groups are vinyl groups such as styrene derivatives, which undergo a radical polymerization upon initiation with a redox initiator. The term “redox” refers to a reactive group that is susceptible to oxidation-reduction activation. The term “vinyl” refers to a reactive group that is activated by a redox initiator, and forms a radical upon reaction. X, Y and Z can be the same or different vinyl groups, for example, methacrylic groups.

For self-reactive compounds containing vinyl reactive groups, the initial environment typically will be an aqueous environment. The modification of the initial environment involves the addition of a redox initiator.

Oxidative Coupling Reactive Groups

In one embodiment of the invention, the reactive groups undergo an oxidative coupling reaction. For example, X, Y and Z can be a halo group such as chloro, with an adjacent electron-withdrawing group on the halogen-bearing carbon (e.g., on the “L” linking group). Exemplary electron-withdrawing groups include nitro, aryl, and so forth.

For such reactive groups, the modification in the initial environment comprises a change in pH. For example, in the presence of a base such as KOH, the self-reactive compounds then undergo a de-hydro, chloro coupling reaction, forming a double bond between the carbon atoms, as illustrated below:

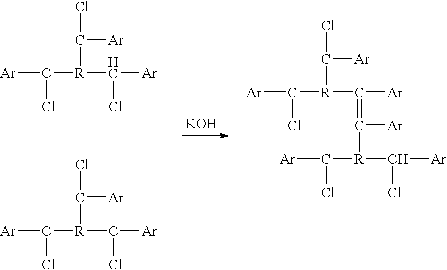

For self-reactive compounds containing oxidative coupling reactive groups, the initial environment typically can be can be dry and sterile, or a non-basic medium. The modification of the initial environment will typically comprise the addition of a base.

Photoinitiated Reactive Groups

In one embodiment of the invention, the reactive groups are photoinitiated groups. For such reactive groups, the modification in the initial environment comprises exposure to ultraviolet radiation.

In one embodiment of the invention, X can be an azide (—N₃) group and Y can be an alkyl group such as —CH(CH₃)₂ or vice versa. Exposure to ultraviolet radiation will then form a bond between the groups to provide for the following linkage: —NH—C(CH₃)₂—CH₂—. In another embodiment of the invention, X can be a benzophenone (—(C₆H₄)—C(O)—(C₆H₅)) group and Y can be an alkyl group such as —CH(CH₃)₂ or vice versa. Exposure to ultraviolet radiation will then form a bond between the groups to provide for the following linkage:

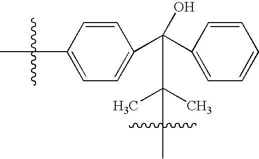

For self-reactive compounds containing photoinitiated reactive groups, the initial environment typically will be in an ultraviolet radiation-shielded environment. This can be for example, storage within a container that is impermeable to ultraviolet radiation.

The modification of the initial environment will typically comprise exposure to ultraviolet radiation.

Temperature-Sensitive Reactive Groups

In one embodiment of the invention, the reactive groups are temperature-sensitive groups, which undergo a thermochemical reaction. For such reactive groups, the modification in the initial environment thus comprises a change in temperature. The term “temperature-sensitive” refers to a reactive group that is chemically inert at one temperature or temperature range and reactive at a different temperature or temperature range.

In one embodiment of the invention, X, Y, and Z are the same or different vinyl groups.

For self-reactive compounds containing reactive groups that are temperature-sensitive, the initial environment typically will be within the range of about 10 to 30° C.

The modification of the initial environment will typically comprise changing the temperature to within the range of about 20 to 40° C.

Linking Groups

The reactive groups may be directly attached to the core, or they may be indirectly attached through a linking group, with longer linking groups also termed “chain extenders.” In the formula (I) shown above, the optional linker groups are represented by L¹, L², and L³, wherein the linking groups are present when p, q and r are equal to 1.

Suitable linking groups are well known in the art. See, for example, WO 97/22371 to Rhee et al. Linking groups are useful to avoid steric hindrance problems that can sometimes associated with the formation of direct linkages between molecules. Linking groups may additionally be used to link several self-reactive compounds together to make larger molecules. In one embodiment, a linking group can be used to alter the degradative properties of the compositions after administration and resultant gel formation. For example, linking groups can be used to promote hydrolysis, to discourage hydrolysis, or to provide a site for enzymatic degradation.

Examples of linking groups that provide hydrolyzable sites, include, inter alia: ester linkages; anhydride linkages, such as those obtained by incorporation of glutarate and succinate; ortho ester linkages; ortho carbonate linkages such as trimethylene carbonate; amide linkages; phosphoester linkages; α-hydroxy acid linkages, such as those obtained by incorporation of lactic acid and glycolic acid; lactone-based linkages, such as those obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, WO 99/07417 to Coury et al. Examples of enzymatically degradable linkages include Leu-Gly-Pro-Ala, which is degraded by collagenase; and Gly-Pro-Lys, which is degraded by plasmin.

Linking groups can also be included to enhance or suppress the reactivity of the various reactive groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophilic group. By contrast, sterically bulky groups in the vicinity of a reactive group can be used to diminish reactivity and thus reduce the coupling rate as a result of steric hindrance.

By way of example, particular linking groups and corresponding formulas are indicated in the following Table 10:

TABLE 10

| Linking group | Component structure |
|---|---|
| —O—(CH₂)ₓ— | —O—(CH₂)ₓ—X |
| | —O—(CH₂)ₓ—Y |
| | —O—(CH₂)ₓ—Z |
| —S—(CH₂)ₓ— | —S—(CH₂)ₓ—X |
| | —S—(CH₂)ₓ—Y |
| | —S—(CH₂)ₓ—Z |
| —NH—(CH₂)ₓ— | —NH—(CH₂)ₓ—X |
| | —NH—(CH₂)ₓ—Y |
| | —NH—(CH₂)ₓ—Z |
| —O—(CO)—NH—(CH₂)ₓ— | —O—(CO)—NH—(CH₂)ₓ—X |
| | —O—(CO)—NH—(CH₂)ₓ—Y |
| | —O—(CO)—NH—(CH₂)ₓ—Z |
| —NH—(CO)—O—(CH₂)ₓ— | —NH—(CO)—O—(CH₂)ₓ—X |
| | —NH—(CO)—O—(CH₂)ₓ—Y |
| | —NH—(CO)—O—(CH₂)ₓ—Z |
| —O—(CO)—(CH₂)ₓ— | —O—(CO)—(CH₂)ₓ—X |
| | —O—(CO)—(CH₂)ₓ—Y |
| | —O—(CO)—(CH₂)ₓ—Z |
| —(CO)—O—(CH₂)ₓ— | —(CO)—O—(CH₂)ₙ—X |
| | —(CO)—O—(CH₂)ₙ—Y |
| | —(CO)—O—(CH₂)ₙ—Z |
| —O—(CO)—O—(CH₂)ₓ— | —O—(CO)—O—(CH₂)ₓ—X |
| | —O—(CO)—O—(CH₂)ₓ—Y |
| | —O—(CO)—O—(CH₂)ₓ—Z |
| —O—(CO)—CHR²— | —O—(CO)—CHR²—X |
| | —O—(CO)—CHR²—Y |
| | —O—(CO)—CHR²—Z |
| —O—R³—(CO)—NH— | —O—R³—(CO)—NH—X |
| | —O—R³—(CO)—NH—Y |
| | —O—R³—(CO)—NH—Z |

In the above Table, x is generally in the range of 1 to about 10; R² is generally hydrocarbyl, typically alkyl or aryl, preferably alkyl, and most preferably lower alkyl; and R³ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows. If a higher molecular weight self-reactive compound is to be used, it will preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

The Core

The “core” of each self-reactive compound is comprised of the molecular structure to which the reactive groups are bound. The molecular core can a polymer, which includes synthetic polymers and naturally occurring polymers. In one embodiment, the core is a polymer containing repeating monomer units. The polymers can be hydrophilic, hydrophobic, or amphiphilic. The molecular core can also be a low molecular weight components such as a C₂₋₁₄ hydrocarbyl or a heteroatom-containing C₂₋₁₄ hydrocarbyl. The heteroatom-containing C₂₋₁₄ hydrocarbyl can have 1 or 2 heteroatoms selected from N, O and S. In a preferred embodiment, the self-reactive compound comprises a molecular core of a synthetic hydrophilic polymer.

Hydrophilic Polymers

As mentioned above, the term “hydrophilic polymer” as used herein refers to a polymer having an average molecular weight and composition that naturally renders, or is selected to render the polymer as a whole “hydrophilic.” Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions.

Synthetic hydrophilic polymers may be homopolymers, block copolymers including di-block and tri-block copolymers, random copolymers, or graft copolymers. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments preferably degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like. Other biodegradable segments that may form part of the hydrophilic polymer core include polyesters such as polylactide, polyethers such as polyalkylene oxide, polyamides such as a protein, and polyurethanes. For example, the core of the self-reactive compound can be a diblock copolymer of tetrafunctionally activated polyethylene glycol and polylactide.

Synthetic hydrophilic polymers that are useful herein include, but are not limited to: polyalkylene oxides, particularly polyethylene glycol (PEG) and poly(ethylene oxide)-poly(propylene oxide) copolymers, including block and random copolymers; polyols such as glycerol, polyglycerol (PG) and particularly highly branched polyglycerol, propylene glycol; poly(oxyalkylene)-substituted diols, and poly(oxyalkylene)-substituted polyols such as mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; poly(acrylic acids) and analogs and copolymers thereof, such as polyacrylic acid per se, polymethacrylic acid, poly(hydroxyethylmethacrylate), poly(hydroxyethylacrylate), poly(methylalkylsulfoxide methacrylates), poly(methylalkylsulfoxide acrylates) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), poly(N-isopropyl-acrylamide), and copolymers thereof, poly(olefinic alcohols) such as poly(vinyl alcohols) and copolymers thereof, poly(N-vinyl lactams) such as poly(vinyl pyrrolidones), poly(N-vinyl caprolactams), and copolymers thereof; polyoxazolines, including poly(methyloxazoline) and poly(ethyloxazoline); and polyvinylamines; as well as copolymers of any of the foregoing. It must be emphasized that the aforementioned list of polymers is not exhaustive, and a variety of other synthetic hydrophilic polymers may be used, as will be appreciated by those skilled in the art.

Those of ordinary skill in the art will appreciate that synthetic polymers such as polyethylene glycol cannot be prepared practically to have exact molecular weights, and that the term “molecular weight” as used herein refers to the weight average molecular weight of a number of molecules in any given sample, as commonly used in the art. Thus, a sample of PEG 2,000 might contain a statistical mixture of polymer molecules ranging in weight from, for example, 1,500 to 2,500 daltons with one molecule differing slightly from the next over a range. Specification of a range of molecular weights indicates that the average molecular weight may be any value between the limits specified, and may include molecules outside those limits. Thus, a molecular weight range of about 800 to about 20,000 indicates an average molecular weight of at least about 800, ranging up to about 20 kDa.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

Although a variety of different synthetic hydrophilic polymers can be used in the present compounds, preferred synthetic hydrophilic polymers are PEG and PG, particularly highly branched PG. Various forms of PEG are extensively used in the modification of biologically active molecules because PEG lacks toxicity, antigenicity, and immunogenicity (i.e., is biocompatible), can be formulated so as to have a wide range of solubilities, and does not typically interfere with the enzymatic activities and/or conformations of peptides. A particularly preferred synthetic hydrophilic polymer for certain applications is a PEG having a molecular weight within the range of about 100 to about 100,000, although for highly branched PEG, far higher molecular weight polymers can be employed, up to 1,000,000 or more, providing that biodegradable sites are incorporated ensuring that all degradation products will have a molecular weight of less than about 30,000. For most PEGs, however, the preferred molecular weight is about 1,000 to about 20,000, more preferably within the range of about 7,500 to about 20,000. Most preferably, the polyethylene glycol has a molecular weight of approximately 10,000.

Naturally occurring hydrophilic polymers include, but are not limited to: proteins such as collagen, fibronectin, albumins, globulins, fibrinogen, fibrin and thrombin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are preferred naturally occurring hydrophilic polymers for use herein.

Unless otherwise specified, the term “collagen” as used herein refers to all forms of collagen, including those, which have been processed or otherwise modified. Thus, collagen from any source may be used in the compounds of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. For example, U.S. Pat. No. 5,428,022 to Palefsky et al. discloses methods of extracting and purifying collagen from the human placenta, and U.S. Pat. No. 5,667,839 to Berg discloses methods of producing recombinant human collagen in the milk of transgenic animals, including transgenic cows. Non-transgenic, recombinant collagen expression in yeast and other cell lines) is described in U.S. Pat. No. 6,413,742 to Olsen et al., U.S. Pat. No. 6,428,978 to Olsen et al., and U.S. Pat. No. 6,653,450 to Berg et al.

Collagen of any type, including, but not limited to, types I, II, III, IV, or any combination thereof, may be used in the compounds of the invention, although type I is generally preferred. Either atelopeptide or telopeptide-containing collagen may be used; however, when collagen from a natural source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the invention, although previously crosslinked collagen may be used.

Collagens for use in the present invention are generally, although not necessarily, in aqueous suspension at a concentration between about 20 mg/ml to about 120 mg/ml, preferably between about 30 mg/ml to about 90 mg/ml. Although intact collagen is preferred, denatured collagen, commonly known as gelatin, can also be used. Gelatin may have the added benefit of being degradable faster than collagen.

Nonfibrillar collagen is generally preferred for use in compounds of the invention, although fibrillar collagens may also be used. The term “nonfibrillar collagen” refers to any modified or unmodified collagen material that is in substantially nonfibrillar form, i.e., molecular collagen that is not tightly associated with other collagen molecules so as to form fibers. Typically, a solution of nonfibrillar collagen is more transparent than is a solution of fibrillar collagen. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559 to Miyata et al. Methylated collagen, which contains reactive amine groups, is a preferred nucleophile-containing component in the compositions of the present invention. In another aspect, methylated collagen is a component that is present in addition to first and second components in the matrix-forming reaction of the present invention. Methylated collagen is described in, for example, in U.S. Pat. No. 5,614,587 to Rhee et al.

Collagens for use in the compositions of the present invention may start out in fibrillar form, then can be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

Fibrillar collagen is less preferred for use in the compounds of the invention. However, as disclosed in U.S. Pat. No. 5,614,587 to Rhee et al., fibrillar collagen, or mixtures of nonfibrillar and fibrillar collagen, may be preferred for use in compounds intended for long-term persistence in vivo.

Hydrophobic Polymers

The core of the self-reactive compound may also comprise a hydrophobic polymer, including low molecular weight polyfunctional species, although for most uses hydrophilic polymers are preferred. Generally, “hydrophobic polymers” herein contain a relatively small proportion of oxygen and/or nitrogen atoms. Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing, for example, multiple nucleophilic groups. Thus, use of short-chain oligomers can avoid solubility-related problems during reaction. Polylactic acid and polyglycolic acid are examples of two particularly suitable hydrophobic polymers.

Amphiphilic Polymers

Generally, amphiphilic polymers have a hydrophilic portion and a hydrophobic (or lipophilic) portion. The hydrophilic portion can be at one end of the core and the hydrophobic portion at the opposite end, or the hydrophilic and hydrophobic portions may be distributed randomly (random copolymer) or in the form of sequences or grafts (block copolymer) to form the amphiphilic polymer core of the self-reactive compound. The hydrophilic and hydrophobic portions may include any of the aforementioned hydrophilic and hydrophobic polymers.

Alternately, the amphiphilic polymer core can be a hydrophilic polymer that has been modified with hydrophobic moieties (e.g., alkylated PEG or a hydrophilic polymer modified with one or more fatty chains), or a hydrophobic polymer that has been modified with hydrophilic moieties (e.g., “PEGylated” phospholipids such as polyethylene glycolated phospholipids).

Low Molecular Weight Components

As indicated above, the molecular core of the self-reactive compound can also be a low molecular weight compound, defined herein as being a C₂₋₁₄ hydrocarbyl or a heteroatom-containing C₂₋₁₄ hydrocarbyl, which contains 1 to 2 heteroatoms selected from N, O, S and combinations thereof. Such a molecular core can be substituted with any of the reactive groups described herein.

Alkanes are suitable C₂₋₁₄ hydrocarbyl molecular cores. Exemplary alkanes, for substituted with a nucleophilic primary amino group and a Y electrophilic group, include, ethyleneamine (H₂N—CH₂CH₂—Y), tetramethyleneamine (H₂N—(CH₄)—Y), pentamethyleneamine (H₂N—(CH₅)—Y), and hexamethyleneamine (H₂N—(CH₆)—Y).

Low molecular weight diols and polyols are also suitable C₂₋₁₄ hydrocarbyls and include trimethylolpropane, di(trimethylol propane), pentaerythritol, and diglycerol. Polyacids are also suitable C₂₋₁₄ hydrocarbyls, and include trimethylolpropane-based tricarboxylic acid, di(trimethylol propane)-based tetracarboxylic acid, heptanedioic acid, octanedioic acid (suberic acid), and hexadecanedioic acid (thapsic acid).

Low molecular weight di- and poly-electrophiles are suitable heteroatom-containing C₂₋₁₄ hydrocarbyl molecular cores. These include, for example, disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives.

In one embodiment of the invention, the self-reactive compound of the invention comprises a low-molecular weight material core, with a plurality of acrylate moieties and a plurality of thiol groups.

Preparation

The self-reactive compounds are readily synthesized to contain a hydrophilic, hydrophobic or amphiphilic polymer core or a low molecular weight core, functionalized with the desired functional groups, i.e., nucleophilic and electrophilic groups, which enable crosslinking. For example, preparation of a self-reactive compound having a polyethylene glycol (PEG) core is discussed below. However, it is to be understood that the following discussion is for purposes of illustration and analogous techniques may be employed with other polymers.

With respect to PEG, first of all, various functionalized PEGs have been used effectively in fields such as protein modification (see Abuchowski et al., Enzymes as Drugs, John Wiley & Sons: New York, N.Y. (1981) pp. 367-383; and Dreborg et al. (1990) Crit. Rev. Therap. Drug Carrier Syst. 6:315), peptide chemistry (see Mutter et al., The Peptides, Academic: New York, N.Y. 2:285-332; and Zalipsky et al. (1987) Int. J. Peptide Protein Res. 30:740), and the synthesis of polymeric drugs (see Zalipsky et al. (1983) Eur. Polym. J. 19:1177; and Ouchi et al. (1987) J. Macromol. Sci. Chem. A24:1011).

Functionalized forms of PEG, including multi-functionalized PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992).

Multi-functionalized forms of PEG are of particular interest and include, PEG succinimidyl glutarate, PEG succinimidyl propionate, succinimidyl butylate, PEG succinimidyl acetate, PEG succinimidyl succinamide, PEG succinimidyl carbonate, PEG propionaldehyde, PEG glycidyl ether, PEG-isocyanate, and PEG-vinylsulfone. Many such forms of PEG are described in U.S. Pat. Nos. 5,328,955 and 6,534,591, both to Rhee et al. Similarly, various forms of multi-amino PEG are commercially available from sources such as PEG Shop, a division of SunBio of South Korea (www.sunbio.com), Nippon Oil and Fats (Yebisu Garden Place Tower, 20-3 Ebisu 4-chome, Shibuya-ku, Tokyo), Nektar Therapeutics (San Carlos, Calif., formerly Shearwater Polymers, Huntsville, Ala.) and from Huntsman's Performance Chemicals Group (Houston, Tex.) under the name Jeffamine® polyoxyalkyleneamines. Multi-amino PEGs useful in the present invention include the Jeffamine diamines (“D” series) and triamines (“T” series), which contain two and three primary amino groups per molecule. Analogous poly(sulfhydryl) PEGs are also available from Nektar Therapeutics, e.g., in the form of pentaerythritol poly(ethylene glycol) ether tetra-sulfhydryl (molecular weight 10,000). These multi-functionalized forms of PEG can then be modified to include the other desired reactive groups.

Reaction with succinimidyl groups to convert terminal hydroxyl groups to reactive esters is one technique for preparing a core with electrophilic groups. This core can then be modified include nucleophilic groups such as primary amines, thiols, and hydroxyl groups. Other agents to convert hydroxyl groups include carbonyldiimidazole and sulfonyl chloride. However, as discussed herein, a wide variety of electrophilic groups may be advantageously employed for reaction with corresponding nucleophilic groups. Examples of such electrophilic groups include acid chloride groups; anhydrides, ketones, aldehydes, isocyanate, isothiocyanate, epoxides, and olefins, including conjugated olefins such as ethenesulfonyl (—SO₂CH═CH₂) and analogous functional groups.

Other In Situ Crosslinking Materials

Numerous other types of in situ forming materials have been described which may be used in combination with an anti-scarring agent in accordance with the invention. The in situ forming material may be a biocompatible crosslinked polymer that is formed from water soluble precursors having electrophilic and nucleophilic groups capable of reacting and crosslinking in situ (see, e.g., U.S. Pat. No. 6,566,406). The in situ forming material may be hydrogel that may be formed through a combination of physical and chemical crosslinking processes, where physical crosslinking is mediated by one or more natural or synthetic components that stabilize the hydrogel-forming precursor solution at a deposition site for a period of time sufficient for more resilient chemical crosslinks to form (see, e.g., U.S. Pat. No. 6,818,018). The in situ forming material may be formed upon exposure to an aqueous fluid from a physiological environment from dry hydrogel precursors (see, e.g., U.S. Pat. No. 6,703,047). The in situ forming material may be a hydrogel matrix that provides controlled release of relatively low molecular weight therapeutic species by first dispersing or dissolving the therapeutic species within relatively hydrophobic rate modifying agents to form a mixture; the mixture is formed into microparticles that are dispersed within bioabsorbable hydrogels, so as to release the water soluble therapeutic agents in a controlled fashion (see, e.g., 6,632,457). The in situ forming material may be a multi-component hydrogel system (see, e.g., U.S. Pat. No. 6,379,373). The in situ forming material may be a multi-arm block copolymer that includes a central core molecule, such as a residue of a polyol, and at least three copolymer arms covalently attached to the central core molecule, each copolymer arm comprising an inner hydrophobic polymer segment covalently attached to the central core molecule and an outer hydrophilic polymer segment covalently attached to the hydrophobic polymer segment, wherein the central core molecule and the hydrophobic polymer segment define a hydrophobic core region (see, e.g., U.S. Pat. No. 6,730,334). The in situ forming material may include a gel-forming macromer that includes at least four polymeric blocks, at least two of which are hydrophobic and at least one of which is hydrophilic, and including a crosslinkable group (see, e.g., U.S. Pat. No. 6,639,014). The in situ forming material may be a water-soluble macromer that includes at least one hydrolysable linkage formed from carbonate or dioxanone groups, at least one water-soluble polymeric block, and at least one polymerizable group (see, e.g., U.S. Pat. No. 6,177,095). The in situ forming material may comprise polyoxyalkylene block copolymers that form weak physical crosslinks to provide gels having a paste-like consistency at physiological temperatures. (see, e.g., U.S. Pat. No. 4,911,926). The in situ forming material may be a thermo-irreversible gel made from polyoxyalkylene polymers and ionic polysaccharides (see, e.g., U.S. Pat. No. 5,126,141). The in situ forming material may be a gel forming composition that includes chitin derivatives (see, e.g., U.S. Pat. No. 5,093,319), chitosan-coagulum (see, e.g., U.S. Pat. No. 4,532,134), or hyaluronic acid (see, e.g., U.S. Pat. No. 4,141,973). The in situ forming material may be an in situ modification of alginate (see, e.g., U.S. Pat. No. 5,266,326). The in situ forming material may be formed from ethylenically unsaturated water soluble macromers that can be crosslinked in contact with tissues, cells, and bioactive molecules to form gels (see, e.g., U.S. Pat. No. 5,573,934). The in situ forming material may include urethane prepolymers used in combination with an unsaturated cyano compound containing a cyano group attached to a carbon atom, such as cyano(meth)acrylic acids and esters thereof (see, e.g., U.S. Pat. No. 4,740,534). The in situ forming material may be a biodegradable hydrogel that polymerizes by a photoinitiated free radical polymerization from water-soluble macromers (see, e.g., U.S. Pat. No. 5,410,016). The in situ forming material may be formed from a two-component mixture including a first part comprising a serum albumin protein in an aqueous buffer having a pH in a range of about 8.0-11.0, and a second part comprising a water-compatible or water-soluble bifunctional crosslinking agent. (see, e.g., U.S. Pat. No. 5,583,114).

In another aspect, in situ forming materials that can be used include those based on the crosslinking of proteins. For example, the in situ forming material may be a biodegradable hydrogel composed of a recombinant or natural human serum albumin and poly(ethylene) glycol polymer solution whereby upon mixing the solution cross-links to form a mechanical non-liquid covering structure which acts as a sealant. See e.g., U.S. Pat. Nos. 6,458,147 and 6,371,975. The in situ forming material may be composed of two separate mixtures based on fibrinogen and thrombin which are dispensed together to form a biological adhesive when intermixed either prior to or on the application site to form a fibrin sealant. See e.g., U.S. Pat. No. 6,764,467. The in situ forming material may be composed of ultrasonically treated collagen and albumin which form a viscous material that develops adhesive properties when crosslinked chemically with glutaraldehyde and amino acids or peptides. See e.g., U.S. Pat. No. 6,310,036. The in situ forming material may be a hydrated adhesive gel composed of an aqueous solution consisting essentially of a protein having amino groups at the side chains (e.g., gelatin, albumin) which is crosslinked with an N-hydroxyimidoester compound. See e.g., U.S. Pat. No. 4,839,345. The in situ forming material may be a hydrogel prepared from a protein or polysaccharide backbone (e.g., albumin or polymannuronic acid) bonded to a cross-linking agent (e.g., polyvalent derivatives of polyethylene or polyalkylene glycol). See e.g., U.S. Pat. No. 5,514,379. The in situ forming material may be composed of a polymerizable collagen composition that is applied to the tissue and then exposed to an initiator to polymerize the collagen to form a seal over a wound opening in the tissue. See e.g., U.S. Pat. No. 5,874,537. The in situ forming material may be a two component mixture composed of a protein (e.g., serum albumin) in an aqueous buffer having a pH in the range of about 8.0-11.0 and a water-soluble bifunctional polyethylene oxide type crosslinking agent, which transforms from a liquid to a strong, flexible bonding composition to seal tissue in situ. See e.g., U.S. Pat. Nos. 5,583,114 and RE38158 and PCT Publication No. WO 96/03159. The in situ forming material may be composed of a protein, a surfactant, and a lipid in a liquid carrier, which is crosslinked by adding a crosslinker and used as a sealant or bonding agent in situ. See e.g., U.S. Patent Application No. 2004/0063613A1 and PCT Publication Nos. WO 01/45761 and WO 03/090683. The in situ forming material may be composed of two enzyme-free liquid components that are mixed by dispensing the components into a catheter tube deployed at the vascular puncture site, wherein, upon mixing, the two liquid components chemically cross-link to form a mechanical non-liquid matrix that seals a vascular puncture site. See e.g., U.S. Patent Application Nos. 2002/0161399A1 and 2001/0018598A1. The in situ forming material may be a cross-linked albumin composition composed of an albumin preparation and a carbodiimide preparation which are mixed under conditions that permit crosslinking of the albumin for use as a bioadhesive or sealant. See e.g., PCT Publication No. WO 99/66964. The in situ forming material may be composed of collagen and a peroxidase and hydrogen peroxide, such that the collagen is crosslinked to from a semi-solid gel that seals a wound. See e.g., PCT Publication No. WO 01/35882.

In another aspect, in situ forming materials that can be used include those based on isocyanate or isothiocyanate capped polymers. For example, the in situ forming material may be composed of isocyanate-capped polymers that are liquid compositions which form into a solid adhesive coating by in situ polymerization and crosslinking upon contact with body fluid or tissue. See e.g., PCT Publication No. WO 04/021983. The in situ forming material may be a moisture-curing sealant composition composed of an active isocyanato-terminated isocyanate prepolymer containing a polyol component with a molecular weight of 2,000 to 20,000 and an isocyanurating catalyst agent. See e.g., U.S. Pat. No. 5,206,331.

In another embodiment, the reagents can undergo an electrophilic-nucleophilic reaction to produce a crosslinked matrix. Polymers containing and/or terminated with nucleophilic groups such as amine, sulfhydryl, hydroxyl, —PH₂ or CO—NH—NH₂ can be used as the nucleophilic reagents and polymers containing and/or terminated with electrophilic groups such as succinimidyl, carboxylic acid, aldehyde, epoxide, isocyanate, vinyl, vinyl sulfone, maleimide, —S—S—(C₅H₄N) or activated esters, such as are used in peptide synthesis can be used as the electrophilic reagents. For example, a 4-armed thiol derivatized poly(ethylene glycol) (e.g., pentaerythritol poly(ethylene glycol)ether tetra-sulfhydryl) can be reacted with a 4 armed NHS-derivatized polyethylene glycol (e.g., pentaerythritol poly(ethylene glycol)ether tetra-succinimidyl glutarate) under basic conditions (pH >about 8). Representative examples of compositions that undergo such electrophilic-nucleophilic crosslinking reactions are described, for example, in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; and PCT Application Publication Nos. WO 04/060405 and WO 04/060346. Other examples of in situ forming materials that can be used include those based on the crosslinking of proteins (described in U.S. Pat. Nos. RE38158; 4,839,345; 5,514,379, 5,583,114; 6,458,147; 6,371,975; U.S. Patent Application Publication Nos. 2002/0161399; 2001/0018598 and PCT Publication Nos. WO 03/090683; WO 01/45761; WO 99/66964 and WO 96/03159).

In another embodiment, the electrophilic- or nucleophilic-terminated polymers can further comprise a polymer that can enhance the mechanical and/or adhesive properties of the in situ forming compositions. This polymer can be a degradable or non-degradable polymer. For example, the polymer may be collagen or a collagen derivative, for example methylated collagen. An example of an in situ forming composition uses pentaerythritol poly(ethylene glycol)ether tetra-sulfhydryl) (4-armed thiol PEG), pentaerythritol poly(ethylene glycol)ether tetra-succinimidyl glutarate) (4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another embodiment, the reagents that can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In the preferred embodiment, the 4 armed NHS-derivatized polyethylene glycol is applied to the tissue under basic conditions (pH >about 8). Other representative examples of compositions of this nature that may be used are disclosed in PCT Application Publication No. WO 04/060405 and WO 04/060346, and U.S. patent application Ser. No. 10/749,123.

In another embodiment, the in situ forming material polymer can be a polyester. Polyesters that can be used in in situ forming compositions include poly(hydroxyesters). In another embodiment, the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one. Representative examples of these types of compositions are described in U.S. Pat. Nos. 5,874,500; 5,936,035; 6,312,725; 6,495,127 and PCT Publication Nos. WO 2004/028547.

In another embodiment, the electrophilic-terminated polymer can be partially or completely replaced by a small molecule or oligomer that comprises an electrophilic group (e.g., disuccinimidyl glutarate).

In another embodiment, the nucleophilic-terminated polymer can be partially or completely replaced by a small molecule or oligomer that comprises a nucleophilic group (e.g., dicysteine, dilysine, trilysine, etc.).

Other examples of in situ forming materials that can be used include those based on the crosslinking of proteins (described in, for example, U.S. Pat. Nos. RE 38158; 4,839,345; 5,514,379, 5,583,114; 6,310,036; 6,458,147; 6,371,975; US Patent Application Publication Nos. 2004/0063613A1, 2002/0161399A1, and 2001/0018598A1, and PCT Publication Nos. WO 03/090683, WO 01/45761, WO 99/66964, and WO 96/03159) and those based on isocyanate or isothiocyanate capped polymers (see, e.g., PCT Publication No. WO 04/021983).

Other examples of in situ forming materials can include reagents that comprise one or more cyanoacrylate groups. These reagents can be used to prepare a poly(alkylcyanoacrylate) or poly(carboxyalkylcyanoacrylate) (e.g., poly(ethylcyanoacrylate), poly(butylcyanoacrylate), poly(isobutylcyanoacrylate), poly(hexylcyanoacrylate), poly(methoxypropylcyanoacrylate), and poly(octylcyanoacrylate)).

Examples of commercially available cyanoacrylates that can be used in the present invention include DERMABOND, INDERMIL, GLUSTITCH, VETBOND, HISTOACRYL, TISSUMEND, HISTOACRYL BLUE and ORABASE SOOTHE-N-SEAL LIQUID PROTECTANT.

In another embodiment, the cyanoacrylate compositions may further comprise additives to stabilize the reagents and/or alter the rate of reaction of the cyanoacrylate, and/or plasticize the poly(cyanoacrylate), and/or alter the rate of degradation of the poly(cyanoacrylate). For example, a trimethylene carbonate based polymer or an oxalate polymer of poly(ethylene glycol) or a ε-caprolactone based copolymer may be mixed with a 2-alkoxyalkylcyanoacrylate (e.g., 2-methoxypropylcyanoacrylate). Representative examples of these compositions are described in U.S. Pat. Nos. 5,350,798 and 6,299,631.

In another embodiment, the cyanoacrylate composition can be prepared by capping heterochain polymers with a cyanoacrylate group. The cyanoacrylate-capped heterochain polymer preferably has at least two cyanoacrylate ester groups per chain. The heterochain polymer can comprise an absorbable poly(ester), poly(ester-carbonate), poly(ether-carbonate) and poly(ether-ester). The poly(ether-ester)s described in U.S. Pat. Nos. 5,653,992 and 5,714,159 can also be used as the heterochain polymers. A triaxial poly(ε-caprolactone-co-trimethylene carbonate) is an example of a poly(ester-carbonate) that can be used. The heterochain polymer may be a polyether. Examples of polyethers that can be used include poly(ethylene glycol), poly(propylene glycol) and block copolymers of poly(ethylene glycol) and poly(propylene glycol) (e.g., PLURONICS group of polymers including but not limited to PLURONIC F127 or F68). Representative examples of these compositions are described in U.S. Pat. No. 6,699,940.

Within another aspect of the invention, fibrosis-inhibiting drug combinations can be delivered with a non-polymeric compound (e.g., a carrier). These non-polymeric carriers can include sucrose derivatives (e.g., sucrose acetate isobutyrate, sucrose oleate), sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydroxyapatite, zeolites; and combinations and mixtures thereof.

Representative examples of patents relating to non-polymeric delivery systems and the preparation include U.S. Pat. Nos. 5,736,152; 5,888,533; 6,120,789; 5,968,542; and 5,747,058.

Within certain embodiments of the invention, the therapeutic compositions are provided that include (i) a fibrosis-inhibiting drug combination. The therapeutic compositions may include one or more additional therapeutic agents (such as described above), for example, anti-inflammatory agents, anti-thrombotic agents, and/or anti-platelet agents. Other agents that may be combined with the therapeutic compositions include, e.g., additional ingredients such as surfactants (e.g., PLURONICS, such as F-127, L-122, L-101, L-92, L-81, and L-61), preservatives, anti-oxidants.

Further, therapeutic compositions and devices of the present invention should preferably have a stable shelf-life of at least several months and be capable of being produced and maintained under sterile conditions. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E.I. DuPont De Nemours and Company, Wilmington, Del.). In a preferred embodiment, the drug-loaded device is terminally sterilized.

In certain embodiments, the present invention provides compositions comprising i) an anti-fibrotic drug combination and ii) a polymer or a compound that forms a polymer in situ. The following are some, but by no means all, of the anti-fibrotic drug combinations and classes of anti-fibrotic drug combinations that may be included in the inventive compositions:

1a. amoxapine and prednisolone,

2a. paroxetine and prednisolone,

3a. dipyridamole and prednisolone,

4a. dexamethasone and econazole,

5a. diflorasone and alprostadil,

6a. dipyridamole and amoxapine,

7a. dipyridamole and ibudilast,

8a. nortriptyline and loratadine (or desloratadine),

9a. albendazole and pentamidine,

10a. itraconazole and lovastatin,

11a. terbinafine and manganese sulfate,

12a. (1) a triazole (e.g., fluconazole or itraconazole) and (2) a diaminopyridine (e.g., phenazopyridine (PZP));

13a. (1) an antiprotozoal (e.g., pentamidine) and (2) a diaminopyridine (e.g., phenazopyridine) or a quaternary ammonium compound (e.g., pentolinium);

14a. (1) an aromatic diamidine and (2) one selected from the group consisting of: (a) an antiestrogen, (b) an anti-fungal imidazole, (d) disulfuram, (e) ribavirin, (f) (i) aminopyridine and (ii) phenothiazine, dacarbazine, or phenelzine, (g) (i) a quaternary ammonium compound and (ii) an anti-fungal imidazole, halopnogin, MnSO₄, or ZnCl₂, (h) (i) an antiestrogen and (ii) phenothiazine, cupric chloride, dacarbazine, methoxsalen, or phenelzine, (j) (i) an anti-fungal imidazone and (ii) disulfuram or ribavirin, and (k) an estrogenic compound and (ii) dacarbazine;

15a. (1) amphotericin B and (2) dithiocarbamoyl disulfide (e.g., disulfuram);

16a. (1) terbinafine and (2) a manganese compound;

17a. (1) a tricyclic antidepressant (TCA) (e.g., amoxapine) and (2) a corticosteroid (e.g., prednisolone);

18a. (1) a tetra-substituted pyrimidopyrimidine (e.g., dipyridamole) and (2) a corticosteroid (e.g., fluorocortisone or prednisolone);

19a. (1) a prostaglandin (e.g., alprostadil) and (2) a retinoid (e.g., tretinoin (vitamin A));

20a. (1) an azole (e.g., imidazone or triazole) and (2) a steroid (e.g., corticosteroids including glucocorticoid or mineralocorticoid);

21a. (1) a steroid and (2) a prostaglandin, beta-adrenergic receptor ligand, anti-mitotic agent, or microtubule inhibitor;

22a. (1) a serotonin norepinephrine reuptake inhibitor (SNRI) or noradrenaline reuptake inhibitor (NARI) and (2) a corticosteroid;

23a. (1) a non-steroidal immunophilin-dependent immunosuppressant (NSIDI) (e.g., calcineurin inhibitor, tacrolimus, ascomycin, pimecrolimus, ISAtx 247) and (2) a non-steroidal immunophilin-dependent immunosuppressant enhancer (NSIDIE) (e.g., selective serotonin reuptake inhibitors, tricyclic antidepressants, phenoxy phenols, anti-histamine, phenothiazines, or mu opioid receptor agonists);

24a. (1) an antihistamines and (2) an additional agent selected from corticosteroids, tricyclic or tetracyclic antidepressants, selective serotonin reuptake inhibitors, and steroid receptor modulators;

25a. (1) a tricyclic compound and (2) a corticosteroid;

26a. (1) an antipsychotic drug (e.g., chlorpromazine) and (2) an antiprotozoal drug (e.g., pentamidine);

27a. (1) an antihelmintic drug (e.g., benzimidazole) and (2) an antiprotozoal drug (e.g., entamidine);

28a. (1) ciclopirox and (2) an antiproliferative agent;

29a. (1) a salicylanilide (e.g., niclosamide) and (2) an antiproliferative agents;

30a. (1) pentamidine or its analogue and (2) chlorpromazine or its analogue;

31a. (1) an antihelmintic drug (e.g., alberdazole, mebendazole, oxibendazole) and (2) an antiprotozoal drug (e.g., pentamidine);

32a. (1) a dibucaine or amide local anaesthetic related to bupivacaine and (2) a vinca alkaloid;

33a. (1) pentamidine, analogue or metabolite thereof and (2) an antiproliferative agent;

34a. (1) a triazole (e.g., itraconazole) and (2) an antiarrhythmic agents (e.g., amiodarone, nicardipine or bepridil);

35a. (1) an azole and (2) an HMG-CoA reductase inhibitor;

36a. a phenothiazine conjugate (e.g., a conjugate of phenothiazine and an antiproliferative agent;

37a. (1) phenothiazine and (2) an antiproliferative agent;

38a. (1) a kinesin inhibitor (e.g., phenothiazine, analog or metabolite) and (2) an antiproliferative agent (e.g., Group A and Group B antiproliferative agents);

39a. (1) an agent that reduces the biological activity of a mitotic kinesin (e.g., chlorpromazine) and (2) an agent that reduces the biological activity of protein tyrosine phosphatase.

As mentioned above, the present invention provides compositions comprising each of the foregoing 47 (i.e., 1a through 47a) listed anti-fibrotic drug combinations or classes of anti-fibrotic drug combinations, with each of the following 97 (i.e., 1b through 97b) polymers and compounds:

1b. A crosslinked polymer.

2b. A polymer that reacts with mammalian tissue.

3b. A polymer that is a naturally occurring polymer.

4b. A polymer that is a protein.

5b. A polymer that is a carbohydrate.

6b. A polymer that is biodegradable.

7b. A polymer that is crosslinked and biodegradable.

8b. A polymer that nonbiodegradable.

9b. Collagen.

10b. Methylated collagen.

11b. Fibrinogen.

12b. Thrombin.

13b. Albumin.

14b. Plasminogen.

15b. von Willebrands factor.

16b. Factor VIII.

17b. Hypoallergenic collagen.

18b. Atelopeptidic collagen.

19b. Telopeptide collagen.

20b. Crosslinked collagen.

21b. Aprotinin.

22b. Gelatin.

23b. A protein conjugate.

24b. A gelatin conjugate.

25b. Hyaluronic acid.

26b. A hyaluronic acid derivative.

27b. A synthetic polymer.

28b. A polymer formed from reactants comprising a synthetic isocyanate-containing compound.

29b. A synthetic isocyanate-containing compound.

30b. A polymer formed from reactants comprising a synthetic thiol-containing compound.

31b. A synthetic thiol-containing compound.

32b. A polymer formed from reactants comprising a synthetic compound containing at least two thiol groups.

33b. A synthetic compound containing at least two thiol groups.

34b. A polymer formed from reactants comprising a synthetic compound containing at least three thiol groups.

35b. A synthetic compound containing at least three thiol groups.

36b. A polymer formed from reactants comprising a synthetic compound containing at least four thiol groups.

37b. A synthetic compound containing at least four thiol groups.

38b. A polymer formed from reactants comprising a synthetic amino-containing compound.

39b. A synthetic amino-containing compound.

40b. A polymer formed from reactants comprising a synthetic compound containing at least two amino groups.

41b. A synthetic compound containing at least two amino groups.

42b. A polymer formed from reactants comprising a synthetic compound containing at least three amino groups.

43b. A synthetic compound containing at least three amino groups.

44b. A polymer formed from reactants comprising a synthetic compound containing at least four amino groups.

45b. A synthetic compound containing at least four amino groups.

46b. A polymer formed from reactants comprising a synthetic compound comprising a carbonyl-oxygen-succinimidyl group.

47b. A synthetic compound comprising a carbonyl-oxygen-succinimidyl group.

48b. A polymer formed from reactants comprising a synthetic compound comprising at least two carbonyl-oxygen-succinimidyl groups.

49b. A synthetic compound comprising at least two carbonyl-oxygen-succinimidyl groups.

50b. A polymer formed from reactants comprising a synthetic compound comprising at least three carbonyl-oxygen-succinimidyl groups.

51b. A synthetic compound comprising at least three carbonyl-oxygen-succinimidyl groups.

52b. A polymer formed from reactants comprising a synthetic compound comprising at least four carbonyl-oxygen-succinimidyl groups.

53b. A synthetic compound comprising at least four carbonyl-oxygen-succinimidyl groups.

54b. A polymer formed from reactants comprising a synthetic polyalkylene oxide-containing compound.

55b. A synthetic polyalkylene oxide-containing compound.

56b. A polymer formed from reactants comprising a synthetic compound comprising both polyalkylene oxide and biodegradable polyester blocks.

57b. A synthetic compound comprising both polyalkylene oxide and biodegradable polyester blocks.

58b. A polymer formed from reactants comprising a synthetic polyalkylene oxide-containing compound having reactive amino groups.

59b. A synthetic polyalkylene oxide-containing compound having reactive amino groups.

60b. A polymer formed from reactants comprising a synthetic polyalkylene oxide-containing compound having reactive thiol groups.

61b. A synthetic polyalkylene oxide-containing compound having reactive thiol groups.

62b. A polymer formed from reactants comprising a synthetic polyalkylene oxide-containing compound having reactive carbonyl-oxygen-succinimidyl groups.

63b. A synthetic polyalkylene oxide-containing compound having reactive carbonyl-oxygen-succinimidyl groups.

64b. A polymer formed from reactants comprising a synthetic compound comprising a biodegradable polyester block.

65b. A synthetic compound comprising a biodegradable polyester block.

66b. A polymer formed from reactants comprising a synthetic polymer formed in whole or part from lactic acid or lactide.

67b. A synthetic polymer formed in whole or part from lactic acid or lactide.

68b. A polymer formed from reactants comprising a synthetic polymer formed in whole or part from glycolic acid or glycolide.

69b. A synthetic polymer formed in whole or part from glycolic acid or glycolide.

70b. A polymer formed from reactants comprising polylysine.

71b. Polylysine.

72b. A polymer formed from reactants comprising (a) protein and (b) a compound comprising a polyalkylene oxide portion.

73b. A polymer formed from reactants comprising (a) protein and (b) polylysine.

74b. A polymer formed from reactants comprising (a) protein and (b) a compound having at least four thiol groups.

75b. A polymer formed from reactants comprising (a) protein and (b) a compound having at least four amino groups.

76b. A polymer formed from reactants comprising (a) protein and (b) a compound having at least four carbonyl-oxygen-succinimide groups.

77b. A polymer formed from reactants comprising (a) protein and (b) a compound having a biodegradable region formed from reactants selected from lactic acid, lactide, glycolic acid, glycolide, and epsilon-caprolactone.

78b. A polymer formed from reactants comprising (a) collagen and (b) a compound comprising a polyalkylene oxide portion.

79b. A polymer formed from reactants comprising (a) collagen and (b) polylysine.

80b. A polymer formed from reactants comprising (a) collagen and (b) a compound having at least four thiol groups.

81b. A polymer formed from reactants comprising (a) collagen and (b) a compound having at least four amino groups.

82b. A polymer formed from reactants comprising (a) collagen and (b) a compound having at least four carbonyl-oxygen-succinimide groups.

83b. A polymer formed from reactants comprising (a) collagen and (b) a compound having a biodegradable region formed from reactants selected from lactic acid, lactide, glycolic acid, glycolide, and epsilon-caprolactone.

84b. A polymer formed from reactants comprising (a) methylated collagen and (b) a compound comprising a polyalkylene oxide portion.

85b. A polymer formed from reactants comprising (a) methylated collagen and (b) polylysine.

86b. A polymer formed from reactants comprising (a) methylated collagen and (b) a compound having at least four thiol groups.

87b. A polymer formed from reactants comprising (a) methylated collagen and (b) a compound having at least four amino groups.

88b. A polymer formed from reactants comprising (a) methylated collagen and (b) a compound having at least four carbonyl-oxygen-succinimide groups.

89b. A polymer formed from reactants comprising (a) methylated collagen and (b) a compound having a biodegradable region formed from reactants selected from lactic acid, lactide, glycolic acid, glycolide, and epsilon-caprolactone.

90b. A polymer formed from reactants comprising hyaluronic acid.

91b. A polymer formed from reactants comprising a hyaluronic acid derivative.

92b. A polymer formed from reactants comprising pentaerythritol poly(ethylene glycol)ether tetra-sulfhydryl of number average molecular weight between 3,000 and 30,000.

93b. Pentaerythritol poly(ethylene glycol)ether tetra-sulfhydryl of number average molecular weight between 3,000 and 30,000.

94b. A polymer formed from reactants comprising pentaerythritol poly(ethylene glycol)ether tetra-amino of number average molecular weight between 3,000 and 30,000.

95b. Pentaerythritol poly(ethylene glycol)ether tetra-amino of number average molecular weight between 3,000 and 30,000.

96b. A polymer formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

97b. A mixture of (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

As mentioned above, the present invention provides compositions comprising each of the foregoing 47 (1a through 47a) listed anti-fibrotic drug combinations or classes of anti-fibrotic drug combinations, with each of the foregoing 97 (1b through 97b) polymers and compounds: Thus, in separate aspects, the invention provides 47 times 97=4,559 described compositions. In other words, each of the following is a distinct aspect of the present invention: 1a+1b; 1a+2b; 1a+3b; 1a+4b; 1a+5b; 1a+6b; 1a+7b; 1a+8b; 1a+9b; 1a+10b; 1a+11b; 1a+12b; 1a+13b; 1a+14b; 1a+15b; 1a+16b; 1a+17b; 1a+18b; 1a+19b; 1a+20b; 1a+21b; 1a+22b; 1a+23b; 1a+24b; 1a+25b; 1a+26b; 1a+27b; 1a+28b; 1a+29b; 1a+30b; 1a+31b; 1a+32b; 1a+33b; 1a+34b; 1a+35b; 1a+36b; 1a+37b; 1a+38b; 1a+39b; 1a+40b; 1a+41b; 1a+42b; 1a+43b; 1a+44b; 1a+45b; 1a+46b; 1a+47b; 1a+48b; 1a+49b; 1a+50b; 1a+51b; 1a+52b; 1a+53b; 1a+54b; 1a+55b; 1a+55b; 1a+57b; 1a+58b; 1a+59b; 1a+60b; 1a+61b; 1a+62b; 1a+63b; 1a+64b; 1a+65b; 1a+66b; 1a+67b; 1a+68b; 1a+69b; 1a+70b; 1a+71b; 1a+72b; 1a+73b; 1a+74b; 1a+75b; 1a+76b; 1a+77b; 1a+78b; 1a+79b; 1a+80b; 1a+81b; 1a+82b; 1a+83b; 1a+84b; 1a+85b; 1a+86b; 1a+87b; 1a+88b; 1a+89b; 1a+90b; 1a+91b; 1a+92b; 1a+93b; 1a+94b; 1a+95b; 1a+96b; 1a+97b; 2a+1b; 2a+2b; 2a+3b; 2a+4b; 2a+5b; 2a+6b; 2a+7b; 2a+8b; 2a+9b; 2a+10b; 2a+11b; 2a+12b; 2a+13b; 2a+14b; 2a+15b; 2a+16b; 2a+17b; 2a+18b; 2a+19b; 2a+20b; 2a+21b; 2a+22b; 2a+23b; 2a+24b; 2a+25b; 2a+26b; 2a+27b; 2a+28b; 2a+29b; 2a+30b; 2a+31b; 2a+32b; 2a+33b; 2a+34b; 2a+35b; 2a+36b; 2a+37b; 2a+38b; 2a+39b; 2a+40b; 2a+41b; 2a+42b; 2a+43b; 2a+44b; 2a+45b; 2a+46b; 2a+47b; 2a+48b; 2a+49b; 2a+50b; 2a+51b; 2a+52b; 2a+53b; 2a+54b; 2a+55b; 2a+55b; 2a+57b; 2a+58b; 2a+59b; 2a+60b; 2a+61b; 2a+62b; 2a+63b; 2a+64b; 2a+65b; 2a+66b; 2a+67b; 2a+68b; 2a+69b; 2a+70b; 2a+71b; 2a+72b; 2a+73b; 2a+74b; 2a+75b; 2a+76b; 2a+77b; 2a+78b; 2a+79b; 2a+80b; 2a+81b; 2a+82b; 2a+83b; 2a+84b; 2a+85b; 2a+86b; 2a+87b; 2a+88b; 2a+89b; 2a+90b; 2a+91b; 2a+92b; 2a+93b; 2a+94b; 2a+95b; 2a+96b; 2a+97b; 3a+22b; 3a+23b; 3a+24b; 3a+25b; 3a+26b; 3a+27b; 3a+28b; 3a+29b; 3a+30b; 3a+31b; 3a+32b; 3a+33b; 3a+34b; 3a+35b; 3a+36b; 3a+37b; 3a+38b; 3a+39b; 3a+40b; 3a+41b; 3a+42b; 3a+43b; 3a+44b; 3a+45b; 3a+46b; 3a+47b; 3a+48b; 3a+49b; 3a+50b; 3a+51b; 3a+52b; 3a+53b; 3a+54b; 3a+55b; 3a+55b; 3a+57b; 3a+58b; 3a+59b; 3a+60b; 3a+61b; 3a+62b; 3a+63b; 3a+64b; 3a+65b; 3a+66b; 3a+67b; 3a+68b; 3a+69b; 3a+70b; 3a+71b; 3a+72b; 3a+73b; 3a+74b; 3a+75b; 3a+76b; 3a+77b; 3a+78b; 3a+79b; 3a+80b; 3a+81b; 3a+82b; 3a+83b; 3a+84b; 3a+85b; 3a+86b; 3a+87b; 3a+88b; 3a+89b; 3a+90b; 3a+91b; 3a+92b; 3a+93b; 3a+94b; 3a+95b; 3a+96b; 3a+97b; 4a+12b; 4a+13b; 4a+14b; 4a+15b; 4a+16b; 4a+17b; 4a+18b; 4a+19b; 4a+20b; 4a+21b; 4a+22b; 4a+23b; 4a+24b; 4a+25b; 4a+26b; 4a+27b; 4a+28b; 4a+29b; 4a+30b; 4a+31b; 4a+32b; 4a+33b; 4a+34b; 4a+35b; 4a+36b; 4a+37b; 4a+38b; 4a+39b; 4a+40b; 4a+41b; 4a+42b; 4a+43b; 4a+44b; 4a+45b; 4a+46b; 4a+47b; 4a+48b; 4a+49b; 4a+50b; 4a+51b; 4a+52b; 4a+53b; 4a+54b; 4a+55b; 4a+55b; 4a+57b; 4a+58b; 4a+59b; 4a+60b; 4a+61b; 4a+62b; 4a+63b; 4a+64b; 4a+65b; 4a+66b; 4a+67b; 4a+68b; 4a+69b; 4a+70b; 4a+71b; 4a+72b; 4a+73b; 4a+74b; 4a+75b; 4a+76b; 4a+77b; 4a+78b; 4a+79b; 4a+80b; 4a+81b; 4a+82b; 4a+83b; 4a+84b; 4a+85b; 4a+86b; 4a+87b; 4a+88b; 4a+89b; 4a+90b; 4a+91b; 4a+92b; 4a+93b; 4a+94b; 4a+95b; 4a+96b; 4a+97b; 5a+12b; 5a+13b; 5a+14b; 5a+15b; 5a+16b; 5a+17b; 5a+18b; 5a+19b; 5a+20b; 5a+21b; 5a+22b; 5a+23b; 5a+24b; 5a+25b; 5a+26b; 5a+27b; 5a+28b; 5a+29b; 5a+30b; 5a+31b; 5a+32b; 5a+33b; 5a+34b; 5a+35b; 5a+36b; 5a+37b; 5a+38b; 5a+39b; 5a+40b; 5a+41b; 5a+42b; 5a+43b; 5a+44b; 5a+45b; 5a+46b; 5a+47b; 5a+48b; 5a+49b; 5a+50b; 5a+51b; 5a+52b; 5a+53b; 5a+54b; 5a+55b; 5a+55b; 5a+57b; 5a+58b; 5a+59b; 5a+60b; 5a+61b; 5a+62b; 5a+63b; 5a+64b; 5a+65b; 5a+66b; 5a+67b; 5a+68b; 5a+69b; 5a+70b; 5a+71b; 5a+72b; 5a+73b; 5a+74b; 5a+75b; 5a+76b; 5a+77b; 5a+78b; 5a+79b; 5a+80b; 5a+81b; 5a+82b; 5a+83b; 5a+84b; 5a+85b; 5a+86b; 5a+87b; 5a+88b; 5a+89b; 5a+90b; 5a+91b; 5a+92b; 5a+93b; 5a+94b; 5a+95b; 5a+96b; 5a+97b; 6a+1b; 6a+2b; 6a+3b; 6a+4b; 6a+5b; 6a+6b; 6a+7b; 6a+8b; 6a+9b; 6a+10b; 6a+11b; 6a+12b; 6a+13b; 6a+14b; 6a+15b; 6a+16b; 6a+17b; 6a+18b; 6a+19b; 6a+20b; 6a+21b; 6a+22b; 6a+23b; 6a+24b; 6a+25b; 6a+26b; 6a+27b; 6a+28b; 6a+29b; 6a+30b; 6a+31b; 6a+32b; 6a+33b; 6a+34b; 6a+35b; 6a+36b; 6a+37b; 6a+38b; 6a+39b; 6a+40b; 6a+41b; 6a+42b; 6a+43b; 6a+44b; 6a+45b; 6a+46b; 6a+47b; 6a+48b; 6a+49b; 6a+50b; 6a+51b; 6a+52b; 6a+53b; 6a+54b; 6a+55b; 6a+55b; 6a+57b; 6a+58b; 6a+59b; 6a+60b; 6a+61b; 6a+62b; 6a+63b; 6a+64b; 6a+65b; 6a+66b; 6a+67b; 6a+68b; 6a+69b; 6a+70b; 6a+71b; 6a+72b; 6a+73b; 6a+74b; 6a+75b; 6a+76b; 6a+77b; 6a+78b; 6a+79b; 6a+80b; 6a+81b; 6a+82b; 6a+83b; 6a+84b; 6a+85b; 6a+86b; 6a+87b; 6a+88b; 6a+89b; 6a+90b; 6a+91b; 6a+92b; 6a+93b; 6a+94b; 6a+95b; 6a+96b; 6a+97b; 7a+1b; 7a+2b; 7a+3b; 7a+4b; 7a+5b; 7a+6b; 7a+7b; 7a+8b; 7a+9b; 7a+10b; 7a+11b; 7a+12b; 7a+13b; 7a+14b; 7a+15b; 7a+16b; 7a+17b; 7a+18b; 7a+19b; 7a+20b; 7a+21b; 7a+22b; 7a+23b; 7a+24b; 7a+25b; 7a+26b; 7a+27b; 7a+28b; 7a+29b; 7a+30b; 7a+31b; 7a+32b; 7a+33b; 7a+34b; 7a+35b; 7a+36b; 7a+37b; 7a+38b; 7a+39b; 7a+40b; 7a+41b; 7a+42b; 7a+43b; 7a+44b; 7a+45b; 7a+46b; 7a+47b; 7a+48b; 7a+49b; 7a+50b; 7a+51b; 7a+52b; 7a+53b; 7a+54b; 7a+55b; 7a+55b; 7a+57b; 7a+58b; 7a+59b; 7a+60b; 7a+61b; 7a+62b; 7a+63b; 7a+64b; 7a+65b; 7a+66b; 7a+67b; 7a+68b; 7a+69b; 7a+70b; 7a+71b; 7a+72b; 7a+73b; 7a+74b; 7a+75b; 7a+76b; 7a+77b; 7a+78b; 7a+79b; 7a+80b; 7a+81b; 7a+82b; 7a+83b; 7a+84b; 7a+85b; 7a+86b; 7a+87b; 7a+88b; 7a+89b; 7a+90b; 7a+91b; 7a+92b; 7a+93b; 7a+94b; 7a+95b; 7a+96b; 7a+97b; 8a+12b; 8a+13b; 8a+14b; 8a+15b; 8a+16b; 8a+17b; 8a+18b; 8a+19b; 8a+20b; 8a+21b; 8a+22b; 8a+23b; 8a+24b; 8a+25b; 8a+26b; 8a+27b; 8a+28b; 8a+29b; 8a+30b; 8a+31b; 8a+32b; 8a+33b; 8a+34b; 8a+35b; 8a+36b; 8a+37b; 8a+38b; 8a+39b; 8a+40b; 8a+41b; 8a+42b; 8a+43b; 8a+44b; 8a+45b; 8a+46b; 8a+47b; 8a+48b; 8a+49b; 8a+50b; 8a+51b; 8a+52b; 8a+53b; 8a+54b; 8a+55b; 8a+55b; 8a+57b; 8a+58b; 8a+59b; 8a+60b; 8a+61b; 8a+62b; 8a+63b; 8a+64b; 8a+65b; 8a+66b; 8a+67b; 8a+68b; 8a+69b; 8a+70b; 8a+71b; 8a+72b; 8a+73b; 8a+74b; 8a+75b; 8a+76b; 8a+77b; 8a+78b; 8a+79b; 8a+80b; 8a+81b; 8a+82b; 8a+83b; 8a+84b; 8a+85b; 8a+86b; 8a+87b; 8a+88b; 8a+89b; 8a+90b; 8a+91b; 8a+92b; 8a+93b; 8a+94b; 8a+95b; 8a+96b; 8a+97b; 9a+1b; 9a+2b; 9a+3b; 9a+4b; 9a+5b; 9a+6b; 9a+7b; 9a+8b; 9a+9b; 9a+10b; 9a+11b; 9a+12b; 9a+13b; 9a+14b; 9a+15b; 9a+16b; 9a+17b; 9a+18b; 9a+19b; 9a+20b; 9a+21b; 9a+22b; 9a+23b; 9a+24b; 9a+25b; 9a+26b; 9a+27b; 9a+28b; 9a+29b; 9a+30b; 9a+31b; 9a+32b; 9a+33b; 9a+34b; 9a+35b; 9a+36b; 9a+37b; 9a+38b; 9a+39b; 9a+40b; 9a+41b; 9a+42b; 9a+43b; 9a+44b; 9a+45b; 9a+46b; 9a+47b; 9a+48b; 9a+49b; 9a+50b; 9a+51b; 9a+52b; 9a+53b; 9a+54b; 9a+55b; 9a+55b; 9a+57b; 9a+58b; 9a+59b; 9a+60b; 9a+61b; 9a+62b; 9a+63b; 9a+64b; 9a+65b; 9a+66b; 9a+67b; 9a+68b; 9a+69b; 9a+70b; 9a+71b; 9a+72b; 9a+73b; 9a+74b; 9a+75b; 9a+76b; 9a+77b; 9a+78b; 9a+79b; 9a+80b; 9a+81b; 9a+82b; 9a+83b; 9a+84b; 9a+85b; 9a+86b; 9a+87b; 9a+88b; 9a+89b; 9a+90b; 9a+91b; 9a+92b; 9a+93b; 9a+94b; 9a+95b; 9a+96b; 9a+97b; 10a+1b; 10a+2b; 10a+3b; 10a+4b; 10a+5b; 10a+6b; 10a+7b; 10a+8b; 10a+9b; 10a+10b; 10a+11b; 10a+12b; 10a+13b; 10a+14b; 10a+15b; 10a+16b; 10a+17b; 10a+18b; 10a+19b; 10a+20b; 10a+21b; 10a+22b; 10a+23b; 10a+24b; 10a+25b; 10a+26b; 10a+27b; 10a+28b; 10a+29b; 10a+30b; 10a+31b; 10a+32b; 10a+33b; 10a+34b; 10a+35b; 10a+36b; 10a+37b; 10a+38b; 10a+39b; 10a+40b; 10a+41b; 10a+42b; 10a+43b; 10a+44b; 10a+45b; 10a+46b; 10a+47b; 10a+48b; 10a+49b; 10a+50b; 10a+51b; 10a+52b; 10a+53b; 10a+54b; 10a+55b; 10a+55b; 10a+57b; 10a+58b; 10a+59b; 10a+60b; 10a+61b; 10a+62b; 10a+63b; 10a+64b; 10a+65b; 10a+66b; 10a+67b; 10a+68b; 10a+69b; 10a+70b; 10a+71b; 10a+72b; 10a+73b; 10a+74b; 10a+75b; 10a+76b; 10a+77b; 10a+78b; 10a+79b; 10a+80b; 10a+81b; 10a+82b; 10a+83b; 10a+84b; 10a+85b; 10a+86b; 10a+87b; 10a+88b; 10a+89b; 10a+90b; 10a+91b; 10a+92b; 10a+93b; 10a+94b; 10a+95b; 10a+96b; 10a+97b; 11a+1b; 11a+2b; 11a+3b; 11a+4b; 11a+5b; 11a+6b; 11a+7b; 11a+8b; 11a+9b; 11a+10b; 11a+11b; 11a+12b; 11a+13b; 11a+14b; 11a+15b; 11a+16b; 11a+17b; 11a+18b; 11a+19b; 11a+20b; 11a+21b; 11a+22b; 11a+23b; 11a+24b; 11a+25b; 11a+26b; 11a+27b; 11a+28b; 11a+29b; 11a+30b; 11a+31b; 11a+32b; 11a+33b; 11a+34b; 11a+35b; 11a+36b; 11a+37b; 11a+38b; 11a+39b; 11a+40b; 11a+41b; 11a+42b; 11a+43b; 11a+44b; 11a+45b; 11a+46b; 11a+47b; 11a+48b; 11a+49b; 11a+50b; 11a+51b; 11a+52b; 11a+53b; 11a+54b; 11a+55b; 11a+55b; 11a+57b; 11a+58b; 11a+59b; 11a+60b; 11a+61b; 11a+62b; 11a+63b; 11a+64b; 11a+65b; 11a+66b; 11a+67b; 11a+68b; 11a+69b; 11a+70b; 11a+71b; 11a+72b; 11a+73b; 11a+74b; 11a+75b; 11a+76b; 11a+77b; 11a+78b; 11a+79b; 11a+80b; 11a+81b; 11a+82b; 11a+83b; 11a+84b; 11a+85b; 11a+86b; 11a+87b; 11a+88b; 11a+89b; 11a+90b; 11a+91b; 11a+92b; 11a+93b; 11a+94b; 11a+95b; 11a+96b; 11a+97b; 12a+1b; 12a+2b; 12a+3b; 12a+4b; 12a+5b; 12a+6b; 12a+7b; 12a+8b; 12a+9b; 12a+10b; 12a+11b; 12a+12b; 12a+13b; 12a+14b; 12a+15b; 12a+16b; 12a+17b; 12a+18b; 12a+19b; 12a+20b; 12a+21b; 12a+22b; 12a+23b; 12a+24b; 12a+25b; 12a+26b; 12a+27b; 12a+28b; 12a+29b; 12a+30b; 12a+31b; 12a+32b; 12a+33b; 12a+34b; 12a+35b; 12a+36b; 12a+37b; 12a+38b; 12a+39b; 12a+40b; 12a+41b; 12a+42b; 12a+43b; 12a+44b; 12a+45b; 12a+46b; 12a+47b; 12a+48b; 12a+49b; 12a+50b; 12a+51b; 12a+52b; 12a+53b; 12a+54b; 12a+55b; 12a+55b; 12a+57b; 12a+58b; 12a+59b; 12a+60b; 12a+61b; 12a+62b; 12a+63b; 12a+64b; 12a+65b; 12a+66b; 12a+67b; 12a+68b; 12a+69b; 12a+70b; 12a+71b; 12a+72b; 12a+73b; 12a+74b; 12a+75b; 12a+76b; 12a+77b; 12a+78b; 12a+79b; 12a+80b; 12a+81b; 12a+82b; 12a+83b; 12a+84b; 12a+85b; 12a+86b; 12a+87b; 12a+88b; 12a+89b; 12a+90b; 12a+91b; 12a+92b; 12a+93b; 12a+94b; 12a+95b; 12a+96b; 12a+97b; 13a+1b; 13a+2b; 13a+3b; 13a+4b; 13a+5b; 13a+6b; 13a+7b; 13a+8b; 13a+9b; 13a+10b; 13a+11b; 13a+12b; 13a+13b; 13a+14b; 13a+15b; 13a+16b; 13a+17b; 13a+18b; 13a+19b; 13a+20b; 13a+21b; 13a+22b; 13a+23b; 13a+24b; 13a+25b; 13a+26b; 13a+27b; 13a+28b; 13a+29b; 13a+30b; 13a+31b; 13a+32b; 13a+33b; 13a+34b; 13a+35b; 13a+36b; 13a+37b; 13a+38b; 13a+39b; 13a+40b; 13a+41b; 13a+42b; 13a+43b; 13a+44b; 13a+45b; 13a+46b; 13a+47b; 13a+48b; 13a+49b; 13a+50b; 13a+51b; 13a+52b; 13a+53b; 13a+54b; 13a+55b; 13a+55b; 13a+57b; 13a+58b; 13a+59b; 13a+60b; 13a+61b; 13a+62b; 13a+63b; 13a+64b; 13a+65b; 13a+66b; 13a+67b; 13a+68b; 13a+69b; 13a+70b; 13a+71b; 13a+72b; 13a+73b; 13a+74b; 13a+75b; 13a+76b; 13a+77b; 13a+78b; 13a+79b; 13a+80b; 13a+81b; 13a+82b; 13a+83b; 13a+84b; 13a+85b; 13a+86b; 13a+87b; 13a+88b; 13a+89b; 13a+90b; 13a+91b; 13a+92b; 13a+93b; 13a+94b; 13a+95b; 13a+96b; 13a+97b; 14a+1b; 14a+2b; 14a+3b; 14a+4b; 14a+5b; 14a+6b; 14a+7b; 14a+8b; 14a+9b; 14a+10b; 14a+11b; 14a+12b; 14a+13b; 14a+14b; 14a+15b; 14a+16b; 14a+17b; 14a+18b; 14a+19b; 14a+20b; 14a+21b; 14a+22b; 14a+23b; 14a+24b; 14a+25b; 14a+26b; 14a+27b; 14a+28b; 14a+29b; 14a+30b; 14a+31b; 14a+32b; 14a+33b; 14a+34b; 14a+35b; 14a+36b; 14a+37b; 14a+38b; 14a+39b; 14a+40b; 14a+41b; 14a+42b; 14a+43b; 14a+44b; 14a+45b; 14a+46b; 14a+47b; 14a+48b; 14a+49b; 14a+50b; 14a+51b; 14a+52b; 14a+53b; 14a+54b; 14a+55b; 14a+55b; 14a+57b; 14a+58b; 14a+59b; 14a+60b; 14a+61b; 14a+62b; 14a+63b; 14a+64b; 14a+65b; 14a+66b; 14a+67b; 14a+68b; 14a+69b; 14a+70b; 14a+71b; 14a+72b; 14a+73b; 14a+74b; 14a+75b; 14a+76b; 14a+77b; 14a+78b; 14a+79b; 14a+80b; 14a+81b; 14a+82b; 14a+83b; 14a+84b; 14a+85b; 14a+86b; 14a+87b; 14a+88b; 14a+89b; 14a+90b; 14a+91b; 14a+92b; 14a+93b; 14a+94b; 14a+95b; 14a+96b; 14a+97b; 15a+1b; 15a+2b; 15a+3b; 15a+4b; 15a+5b; 15a+6b; 15a+7b; 15a+8b; 15a+9b; 15a+10b; 15a+11b; 15a+12b; 15a+13b; 15a+14b; 15a+15b; 15a+16b; 15a+17b; 15a+18b; 15a+19b; 15a+20b; 15a+21b; 15a+22b; 15a+23b; 15a+24b; 15a+25b; 15a+26b; 15a+27b; 15a+28b; 15a+29b 15a+30b; 15a+31b; 15a+32b; 15a+33b; 15a+34b; 15a+35b; 15a+36b; 15a+37b; 15a+38b; 15a+39b; 15a+40b; 15a+41b; 15a+42b; 15a+43b; 15a+44b; 15a+45b; 15a+46b; 15a+47b; 15a+48b; 15a+49b; 15a+50b; 15a+51b; 15a+52b; 15a+53b; 15a+54b; 15a+55b; 15a+55b; 15a+57b; 15a+58b; 15a+59b; 15a+60b; 15a+61b; 15a+62b; 15a+63b; 15a+64b; 15a+65b; 15a+66b; 15a+67b; 15a+68b; 15a+69b; 15a+70b; 15a+71b; 15a+72b; 15a+73b; 15a+74b; 15a+75b; 15a+76b; 15a+77b; 15a+78b; 15a+79b; 15a+80b; 15a+81b; 15a+82b; 15a+83b; 15a+84b; 15a+85b; 15a+86b; 15a+87b; 15a+88b; 15a+89b; 15a+90b; 15a+91b; 15a+92b; 15a+93b; 15a+94b; 15a+95b; 15a+96b; 15a+97b; 16a+1b; 16a+2b; 16a+3b; 16a+4b; 16a+5b; 16a+6b; 16a+7b; 16a+8b; 16a+9b; 16a+10b; 16a+11b; 16a+12b; 16a+13b; 16a+14b; 16a+15b; 16a+16b; 16a+17b; 16a+18b; 16a+19b; 16a+20b; 16a+21b; 16a+22b; 16a+23b; 16a+24b; 16a+25b; 16a+26b; 16a+27b; 16a+28b; 16a+29b; 16a+30b; 16a+31b; 16a+32b; 16a+33b; 16a+34b; 16a+35b; 16a+36b; 16a+37b; 16a+38b; 16a+39b; 16a+40b; 16a+41b; 16a+42b; 16a+43b; 16a+44b; 16a+45b; 16a+46b; 16a+47b; 16a+48b; 16a+49b; 16a+50b; 16a+51b; 16a+52b; 16a+53b; 16a+54b; 16a+55b; 16a+55b; 16a+57b; 16a+58b; 16a+59b; 16a+60b; 16a+61b; 16a+62b; 16a+63b; 16a+64b; 16a+65b; 16a+66b; 16a+67b; 16a+68b; 16a+69b; 16a+70b; 16a+71b; 16a+72b; 16a+73b; 16a+74b; 16a+75b; 16a+76b; 16a+77b; 16a+78b; 16a+79b; 16a+80b; 16a+81b; 16a+82b; 16a+83b; 16a+84b; 16a+85b; 16a+86b; 16a+87b; 16a+88b; 16a+89b; 16a+90b; 16a+91b; 16a+92b; 16a+93b; 16a+94b; 16a+95b; 16a+96b; 16a+97b; 17a+1b; 17a+2b; 17a+3b; 17a+4b; 17a+5b; 17a+6b; 17a+7b; 17a+8b; 17a+9b; 17a+10b; 17a+11b; 17a+12b; 17a+13b; 17a+14b; 17a+15b; 17a+16b; 17a+17b; 17a+18b; 17a+19b; 17a+20b; 17a+21b; 17a+22b; 17a+23b; 17a+24b; 17a+25b; 17a+26b; 17a+27b; 17a+28b; 17a+29b; 17a+30b; 17a+31b; 17a+32b; 17a+33b; 17a+34b; 17a+35b; 17a+36b; 17a+37b; 17a+38b; 17a+39b; 17a+40b; 17a+41b; 17a+42b; 17a+43b; 17a+44b; 17a+45b; 17a+46b; 17a+47b; 17a+48b; 17a+49b; 17a+50b; 17a+51b; 17a+52b; 17a+53b; 17a+54b; 17a+55b; 17a+55b; 17a+57b; 17a+58b; 17a+59b; 17a+60b; 17a+61b; 17a+62b; 17a+63b; 17a+64b; 17a+65b; 17a+66b; 17a+67b; 17a+68b; 17a+69b; 17a+70b; 17a+71b; 17a+72b; 17a+73b; 17a+74b; 17a+75b; 17a+76b; 17a+77b; 17a+78b; 17a+79b; 17a+80b; 17a+81b; 17a+82b; 17a+83b; 17a+84b; 17a+85b; 17a+86b; 17a+87b; 17a+88b; 17a+89b; 17a+90b; 17a+91b; 17a+92b; 17a+93b; 17a+94b; 17a+95b; 17a+96b; 17a+97b; 18a+1b; 18a+2b; 18a+3b; 18a+4b; 18a+5b; 18a+6b; 18a+7b; 18a+8b; 18a+9b; 18a+10b; 18a+11b; 18a+12b; 18a+13b; 18a+14b; 18a+15b; 18a+16b; 18a+17b; 18a+18b; 18a+19b; 18a+20b; 18a+21b; 18a+22b; 18a+23b; 18a+24b; 18a+25b; 18a+26b; 18a+27b; 18a+28b; 18a+29b; 18a+30b; 18a+31b; 18a+32b; 18a+33b; 18a+34b; 18a+35b; 18a+36b; 18a+37b; 18a+38b; 18a+39b; 18a+40b; 18a+41b; 18a+42b; 18a+43b; 18a+44b; 18a+45b; 18a+46b; 18a+47b; 18a+48b; 18a+49b; 18a+50b; 18a+51b; 18a+52b; 18a+53b; 18a+54b; 18a+55b; 18a+55b; 18a+57b; 18a+58b; 18a+59b; 18a+60b; 18a+61b; 18a+62b; 18a+63b; 18a+64b; 18a+65b; 18a+66b; 18a+67b; 18a+68b; 18a+69b; 18a+70b; 18a+71b; 18a+72b; 18a+73b; 18a+74b; 18a+75b; 18a+76b; 18a+77b; 18a+78b; 18a+79b; 18a+80b; 18a+81b; 18a+82b; 18a+83b; 18a+84b; 18a+85b; 18a+86b; 18a+87b; 18a+88b; 18a+89b; 18a+90b; 18a+91b; 18a+92b; 18a+93b; 18a+94b; 18a+95b; 18a+96b; 18a+97b; 19a+1b; 19a+2b; 19a+3b; 19a+4b; 19a+5b; 19a+6b; 19a+7b; 19a+8b; 19a+9b; 19a+10b; 19a+11b; 19a+12b; 19a+13b; 19a+14b; 19a+15b; 19a+16b; 19a+17b; 19a+18b; 19a+19b; 19a+20b; 19a+21b; 19a+22b; 19a+23b; 19a+24b; 19a+25b; 19a+26b; 19a+27b; 19a+28b; 19a+29b; 19a+30b; 19a+31b; 19a+32b; 19a+33b; 19a+34b; 19a+35b; 19a+36b; 19a+37b; 19a+38b; 19a+39b; 19a+40b; 19a+41b; 19a+42b; 19a+43b; 19a+44b; 19a+45b; 19a+46b; 19a+47b; 19a+48b; 19a+49b; 19a+50b; 19a+51b; 19a+52b; 19a+53b; 19a+54b; 19a+55b; 19a+55b; 19a+57b; 19a+58b; 19a+59b; 19a+60b; 19a+61b; 19a+62b; 19a+63b; 19a+64b; 19a+65b; 19a+66b; 19a+67b; 19a+68b; 19a+69b; 19a+70b; 19a+71b; 19a+72b; 19a+73b; 19a+74b; 19a+75b; 19a+76b; 19a+77b; 19a+78b; 19a+79b; 19a+80b; 19a+81b; 19a+82b; 19a+83b; 19a+84b; 19a+85b; 19a+86b; 19a+87b; 19a+88b; 19a+89b; 19a+90b; 19a+91b; 19a+92b; 19a+93b; 19a+94b; 19a+95b; 19a+96b; 19a+97b; 20a+1b; 20a+2b; 20a+3b; 20a+4b; 20a+5b; 20a+6b; 20a+7b; 20a+8b; 20a+9b; 20a+10b; 20a+11b; 20a+12b; 20a+13b; 20a+14b; 20a+15b; 20a+16b; 20a+17b; 20a+18b; 20a+19b; 20a+20b; 20a+21b; 20a+22b; 20a+23b; 20a+24b; 20a+25b; 20a+26b; 20a+27b; 20a+28b; 20a+29b; 20a+30b; 20a+31b; 20a+32b; 20a+33b; 20a+34b; 20a+35b; 20a+36b; 20a+37b; 20a+38b; 20a+39b; 20a+40b; 20a+41b; 20a+42b; 20a+43b; 20a+44b; 20a+45b; 20a+46b; 20a+47b; 20a+48b; 20a+49b; 20a+50b; 20a+51b; 20a+52b; 20a+53b; 20a+54b; 20a+55b; 20a+55b; 20a+57b; 20a+58b; 20a+59b; 20a+60b; 20a+61b; 20a+62b; 20a+63b; 20a+64b; 20a+65b; 20a+66b; 20a+67b; 20a+68b; 20a+69b; 20a+70b; 20a+71b; 20a+72b; 20a+73b; 20a+74b; 20a+75b; 20a+76b; 20a+77b; 20a+78b; 20a+79b; 20a+80b; 20a+81b; 20a+82b; 20a+83b; 20a+84b; 20a+85b; 20a+86b; 20a+87b; 20a+88b; 20a+89b; 20a+90b; 20a+91b; 20a+92b; 20a+93b; 20a+94b; 20a+95b; 20a+96b; 20a+97b; 21a+1b; 21a+2b; 21a+3b; 21a+4b; 21a+5b; 21a+6b; 21a+7b; 21a+8b; 21a+9b; 21a+10b; 21a+11b; 21a+12b; 21a+13b; 21a+14b; 21a+15b; 21a+16b; 21a+17b; 21a+18b; 21a+19b; 21a+20b; 21a+21b; 21a+22b; 21a+23b; 21a+24b; 21a+25b; 21a+26b; 21a+27b; 21a+28b; 21a+29b; 21a+30b; 21a+31b; 21a+32b; 21a+33b; 21a+34b; 21a+35b; 21a+36b; 21a+37b; 21a+38b; 21a+39b; 21a+40b; 21a+41b; 21a+42b; 21a+43b; 21a+44b; 21a+45b; 21a+46b; 21a+47b; 21a+48b; 21a+49b; 21a+50b; 21a+51b; 21a+52b; 21a+53b; 21a+54b; 21a+55b; 21a+55b; 21a+57b; 21a+58b; 21a+59b; 21a+60b; 21a+61b; 21a+62b; 21a+63b; 21a+64b; 21a+65b; 21a+66b; 21a+67b; 21a+68b; 21a+69b; 21a+70b; 21a+71b; 21a+72b; 21a+73b; 21a+74b; 21a+75b; 21a+76b; 21a+77b; 21a+78b; 21a+79b; 21a+80b; 21a+81b; 21a+82b; 21a+83b; 21a+84b; 21a+85b; 21a+86b; 21a+87b; 21a+88b; 21a+89b; 21a+90b; 21a+91b; 21a+92b; 21a+93b; 21a+94b; 21a+95b; 21a+96b; 21a+97b; 22a+1b; 22a+2b; 22a+3b; 22a+4b; 22a+5b; 22a+6b; 22a+7b; 22a+8b; 22a+9b; 22a+10b; 22a+11b; 22a+12b; 22a+13b; 22a+14b; 22a+15b; 22a+16b; 22a+17b; 22a+18b; 22a+19b; 22a+20b; 22a+21b; 22a+22b; 22a+23b; 22a+24b; 22a+25b; 22a+26b; 22a+27b; 22a+28b; 22a+29b; 22a+30b; 22a+31b; 22a+32b; 22a+33b; 22a+34b; 22a+35b; 22a+36b; 22a+37b; 22a+38b; 22a+39b; 22a+40b; 22a+41b; 22a+42b; 22a+43b; 22a+44b; 22a+45b; 22a+46b; 22a+47b; 22a+48b; 22a+49b; 22a+50b; 22a+51b; 22a+52b; 22a+53b; 22a+54b; 22a+55b; 22a+55b; 22a+57b; 22a+58b; 22a+59b; 22a+60b; 22a+61b; 22a+62b; 22a+63b; 22a+64b; 22a+65b; 22a+66b; 22a+67b; 22a+68b; 22a+69b; 22a+70b; 22a+71b; 22a+72b; 22a+73b; 22a+74b; 22a+75b; 22a+76b; 22a+77b; 22a+78b; 22a+79b; 22a+80b; 22a+81b; 22a+82b; 22a+83b; 22a+84b; 22a+85b; 22a+86b; 22a+87b; 22a+88b; 22a+89b; 22a+90b; 22a+91b; 22a+92b; 22a+93b; 22a+94b; 22a+95b; 22a+96b; 22a+97b; 23a+1b; 23a+2b; 23a+3b; 23a+4b; 23a+5b; 23a+6b; 23a+7b; 23a+8b; 23a+9b; 23a+10b; 23a+11b; 23a+12b; 23a+13b; 23a+14b; 23a+15b; 23a+16b; 23a+17b; 23a+18b; 23a+19b; 23a+20b; 23a+21b; 23a+22b; 23a+23b; 23a+24b; 23a+25b; 23a+26b; 23a+27b; 23a+28b; 23a+29b; 23a+30b; 23a+31b; 23a+32b; 23a+33b; 23a+34b; 23a+35b; 23a+36b; 23a+37b; 23a+38b; 23a+39b; 23a+40b; 23a+41b; 23a+42b; 23a+43b; 23a+44b; 23a+45b; 23a+46b; 23a+47b; 23a+48b; 23a+49b; 23a+50b; 23a+51b; 23a+52b; 23a+53b; 23a+54b; 23a+55b; 23a+55b; 23a+57b; 23a+58b; 23a+59b; 23a+60b; 23a+61b; 23a+62b; 23a+63b; 23a+64b; 23a+65b; 23a+66b; 23a+67b; 23a+68b; 23a+69b; 23a+70b; 23a+71b; 23a+72b; 23a+73b; 23a+74b; 23a+75b; 23a+76b; 23a+77b; 23a+78b; 23a+79b; 23a+80b; 23a+81b; 23a+82b; 23a+83b; 23a+84b; 23a+85b; 23a+86b; 23a+87b; 23a+88b; 23a+89b; 23a+90b; 23a+91b; 23a+92b; 23a+93b; 23a+94b; 23a+95b; 23a+96b; 23a+97b; 24a+1b; 24a+2b; 24a+3b; 24a+4b; 24a+5b; 24a+6b; 24a+7b; 24a+8b; 24a+9b; 24a+10b; 24a+11b; 24a+12b; 24a+13b; 24a+14b; 24a+15b; 24a+16b; 24a+17b; 24a+18b; 24a+19b; 24a+20b; 24a+21b; 24a+22b; 24a+23b; 24a+24b; 24a+25b; 24a+26b; 24a+27b; 24a+28b; 24a+29b; 24a+30b; 24a+31b; 24a+32b; 24a+33b; 24a+34b; 24a+35b; 24a+36b; 24a+37b; 24a+38b; 24a+39b; 24a+40b; 24a+41b; 24a+42b; 24a+43b; 24a+44b; 24a+45b; 24a+46b; 24a+47b; 24a+48b; 24a+49b; 24a+50b; 24a+51b; 24a+52b; 24a+53b; 24a+54b; 24a+55b; 24a+55b; 24a+57b; 24a+58b; 24a+59b; 24a+60b; 24a+61b; 24a+62b; 24a+63b; 24a+64b; 24a+65b; 24a+66b; 24a+67b; 24a+68b; 24a+69b; 24a+70b; 24a+71b; 24a+72b; 24a+73b; 24a+74b; 24a+75b; 24a+76b; 24a+77b; 24a+78b; 24a+79b; 24a+80b; 24a+81b; 24a+82b; 24a+83b; 24a+84b; 24a+85b; 24a+86b; 24a+87b; 24a+88b; 24a+89b; 24a+90b; 24a+91b; 24a+92b; 24a+93b; 24a+94b; 24a+95b; 24a+96b; 24a+97b; 25a+1b; 25a+2b; 25a+3b; 25a+4b; 25a+5b; 25a+6b; 25a+7b; 25a+8b; 25a+9b; 25a+10b; 25a+11b; 25a+12b; 25a+13b; 25a+14b; 25a+15b; 25a+16b; 25a+17b; 25a+18b; 25a+19b; 25a+20b; 25a+21b; 25a+22b; 25a+23b; 25a+24b; 25a+25b; 25a+26b; 25a+27b; 25a+28b; 25a+29b; 25a+30b; 25a+31b; 25a+32b; 25a+33b; 25a+34b; 25a+35b; 25a+36b; 25a+37b; 25a+38b; 25a+39b; 25a+40b; 25a+41b; 25a+42b; 25a+43b; 25a+44b; 25a+45b; 25a+46b; 25a+47b; 25a+48b; 25a+49b; 25a+50b; 25a+51b; 25a+52b; 25a+53b; 25a+54b; 25a+55b; 25a+55b; 25a+57b; 25a+58b; 25a+59b; 25a+60b; 25a+61b; 25a+62b; 25a+63b; 25a+64b; 25a+65b; 25a+66b; 25a+67b; 25a+68b; 25a+69b; 25a+70b; 25a+71b; 25a+72b; 25a+73b; 25a+74b; 25a+75b; 25a+76b; 25a+77b; 25a+78b; 25a+79b; 25a+80b; 25a+81b; 25a+82b; 25a+83b; 25a+84b; 25a+85b; 25a+86b; 25a+87b; 25a+88b; 25a+89b; 25a+90b; 25a+91b; 25a+92b; 25a+93b; 25a+94b; 25a+95b; 25a+96b; 25a+97b; 26a+1b; 26a+2b; 26a+3b; 26a+4b; 26a+5b; 26a+6b; 26a+7b; 26a+8b; 26a+9b; 26a+10b; 26a+11b; 26a+12b; 26a+13b; 26a+14b; 26a+15b; 26a+16b; 26a+17b; 26a+18b; 26a+19b; 26a+20b; 26a+21b; 26a+22b; 26a+23b; 26a+24b; 26a+25b; 26a+26b; 26a+27b; 26a+28b; 26a+29b; 26a+30b; 26a+31b; 26a+32b; 26a+33b; 26a+34b; 26a+35b; 26a+36b; 26a+37b; 26a+38b; 26a+39b; 26a+40b; 26a+41b; 26a+42b; 26a+43b; 26a+44b; 26a+45b; 26a+46b; 26a+47b; 26a+48b; 26a+49b; 26a+50b; 26a+51b; 26a+52b; 26a+53b; 26a+54b; 26a+55b; 26a+55b; 26a+57b; 26a+58b; 26a+59b; 26a+60b; 26a+61b; 26a+62b; 26a+63b; 26a+64b; 26a+65b; 26a+66b; 26a+67b; 26a+68b; 26a+69b; 26a+70b; 26a+71b; 26a+72b; 26a+73b; 26a+74b; 26a+75b; 26a+76b; 26a+77b; 26a+78b; 26a+79b; 26a+80b; 26a+81b; 26a+82b; 26a+83b; 26a+84b; 26a+85b; 26a+86b; 26a+87b; 26a+88b; 26a+89b; 26a+90b; 26a+91b; 26a+92b; 26a+93b; 26a+94b; 26a+95b; 26a+96b; 26a+97b; 27a+1b; 27a+2b; 27a+3b; 27a+4b; 27a+5b; 27a+6b; 27a+7b; 27a+8b; 27a+9b; 27a+10b; 27a+11b; 27a+12b; 27a+13b; 27a+14b; 27a+15b; 27a+16b; 27a+17b; 27a+18b; 27a+19b; 27a+20b; 27a+21b; 27a+22b; 27a+23b; 27a+24b; 27a+25b; 27a+26b; 27a+27b; 27a+28b; 27a+29b; 27a+30b; 27a+31b; 27a+32b; 27a+33b; 27a+34b; 27a+35b; 27a+36b; 27a+37b; 27a+38b; 27a+39b; 27a+40b; 27a+41b; 27a+42b; 27a+43b; 27a+44b; 27a+45b; 27a+46b; 27a+47b; 27a+48b; 27a+49b; 27a+50b; 27a+51b; 27a+52b; 27a+53b; 27a+54b; 27a+55b; 27a+55b; 27a+57b; 27a+58b; 27a+59b; 27a+60b; 27a+61b; 27a+62b; 27a+63b; 27a+64b; 27a+65b; 27a+66b; 27a+67b; 27a+68b; 27a+69b; 27a+70b; 27a+71b; 27a+72b; 27a+73b; 27a+74b; 27a+75b; 27a+76b; 27a+77b; 27a+78b; 27a+79b; 27a+80b; 27a+81b; 27a+82b; 27a+83b; 27a+84b; 27a+85b; 27a+86b; 27a+87b; 27a+88b; 27a+89b; 27a+90b; 27a+91b; 27a+92b; 27a+93b; 27a+94b; 27a+95b; 27a+96b; 27a+97b; 28a+1b; 28a+2b; 28a+3b; 28a+4b; 28a+5b; 28a+6b; 28a+7b; 28a+8b; 28a+9b; 28a+10b; 28a+11b; 28a+12b; 28a+13b; 28a+14b; 28a+15b; 28a+16b; 28a+17b; 28a+18b; 28a+19b; 28a+20b; 28a+21b; 28a+22b; 28a+23b; 28a+24b; 28a+25b; 28a+26b; 28a+27b; 28a+28b; 28a+29b; 28a+30b; 28a+31b; 28a+32b; 28a+33b; 28a+34b; 28a+35b; 28a+36b; 28a+37b; 28a+38b; 28a+39b; 28a+40b; 28a+41b; 28a+42b; 28a+43b; 28a+44b; 28a+45b; 28a+46b; 28a+47b; 28a+48b; 28a+49b; 28a+50b; 28a+51b; 28a+52b; 28a+53b; 28a+54b; 28a+55b; 28a+55b; 28a+57b; 28a+58b; 28a+59b; 28a+60b; 28a+61b; 28a+62b; 28a+63b; 28a+64b; 28a+65b; 28a+66b; 28a+67b; 28a+68b; 28a+69b; 28a+70b; 28a+71b; 28a+72b; 28a+73b; 28a+74b; 28a+75b; 28a+76b; 28a+77b; 28a+78b; 28a+79b; 28a+80b; 28a+81b; 28a+82b; 28a+83b; 28a+84b; 28a+85b; 28a+86b; 28a+87b; 28a+88b; 28a+89b; 28a+90b; 28a+91b; 28a+92b; 28a+93b; 28a+94b; 28a+95b; 28a+96b; 28a+97b; 29a+1b; 29a+2b; 29a+3b; 29a+4b; 29a+5b; 29a+6b; 29a+7b; 29a+8b; 29a+9b; 29a+10b; 29a+11b; 29a+12b; 29a+13b; 29a+14b; 29a+15b; 29a+16b; 29a+17b; 29a+18b; 29a+19b; 29a+20b; 29a+21b; 29a+22b; 29a+23b; 29a+24b; 29a+25b; 29a+26b; 29a+27b; 29a+28b; 29a+29b; 29a+30b; 29a+31b; 29a+32b; 29a+33b; 29a+34b; 29a+35b; 29a+36b; 29a+37b; 29a+38b; 29a+39b; 29a+40b; 29a+41b; 29a+42b; 29a+43b; 29a+44b; 29a+45b; 29a+46b; 29a+47b; 29a+48b; 29a+49b; 29a+50b; 29a+51b; 29a+52b; 29a+53b; 29a+54b; 29a+55b; 29a+55b; 29a+57b; 29a+58b; 29a+59b; 29a+60b; 29a+61b; 29a+62b; 29a+63b; 29a+64b; 29a+65b; 29a+66b; 29a+67b; 29a+68b; 29a+69b; 29a+70b; 29a+71b; 29a+72b; 29a+73b; 29a+74b; 29a+75b; 29a+76b; 29a+77b; 29a+78b; 29a+79b; 29a+80b; 29a+81b; 29a+82b; 29a+83b; 29a+84b; 29a+85b; 29a+86b; 29a+87b; 29a+88b; 29a+89b; 29a+90b; 29a+91b; 29a+92b; 29a+93b; 29a+94b; 29a+95b; 29a+96b; 29a+97b; 30a+1b; 30a+2b; 30a+3b; 30a+4b; 30a+5b; 30a+6b; 30a+7b; 30a+8b; 30a+9b; 30a+10b; 30a+11b; 30a+12b; 30a+13b; 30a+14b; 30a+15b; 30a+16b; 30a+17b; 30a+18b; 30a+19b; 30a+20b; 30a+21b; 30a+22b; 30a+23b; 30a+24b; 30a+25b; 30a+26b; 30a+27b; 30a+28b; 30a+29b; 30a+30b; 30a+31b; 30a+32b; 30a+33b; 30a+34b; 30a+35b; 30a+36b; 30a+37b; 30a+38b; 30a+39b; 30a+40b; 30a+41b; 30a+42b; 30a+43b; 30a+44b; 30a+45b; 30a+46b; 30a+47b; 30a+48b; 30a+49b; 30a+50b; 30a+51b; 30a+52b; 30a+53b; 30a+54b; 30a+55b; 30a+55b; 30a+57b; 30a+58b; 30a+59b; 30a+60b; 30a+61b; 30a+62b; 30a+63b; 30a+64b; 30a+65b; 30a+66b; 30a+67b; 30a+68b; 30a+69b; 30a+70b; 30a+71b; 30a+72b; 30a+73b; 30a+74b; 30a+75b; 30a+76b; 30a+77b; 30a+78b; 30a+79b; 30a+80b; 30a+81b; 30a+82b; 30a+83b; 30a+84b; 30a+85b; 30a+86b; 30a+87b; 30a+88b; 30a+89b; 30a+90b; 30a+91b; 30a+92b; 30a+93b; 30a+94b; 30a+95b; 30a+96b; 30a+97b; 31a+1b; 31a+2b; 31a+3b; 31a+4b; 31a+5b; 31a+6b; 31a+7b; 31a+8b; 31a+9b; 31a+10b; 31a+11b; 31a+12b; 31a+13b; 31a+14b; 31a+15b; 31a+16b; 31a+17b; 31a+18b; 31a+19b; 31a+20b; 31a+21b; 31a+22b; 31a+23b; 31a+24b; 31a+25b; 31a+26b; 31a+27b; 31a+28b; 31a+29b; 31a+30b; 31a+31b; 31a+32b; 31a+33b; 31a+34b; 31a+35b; 31a+36b; 31a+37b; 31a+38b; 31a+39b; 31a+40b; 31a+41b; 31a+42b; 31a+43b; 31a+44b; 31a+45b; 31a+46b; 31a+47b; 31a+48b; 31a+49b; 31a+50b; 31a+51b; 31a+52b; 31a+53b; 31a+54b; 31a+55b; 31a+55b; 31a+57b; 31a+58b; 31a+59b; 31a+60b; 31a+61b; 31a+62b; 31a+63b; 31a+64b; 31a+65b; 31a+66b; 31a+67b; 31a+68b; 31a+69b; 31a+70b; 31a+71b; 31a+72b; 31a+73b; 31a+74b; 31a+75b; 31a+76b; 31a+77b; 31a+78b; 31a+79b; 31a+80b; 31a+81b; 31a+82b; 31a+83b; 31a+84b; 31a+85b; 31a+86b; 31a+87b; 31a+88b; 31a+89b; 31a+90b; 31a+91b; 31a+92b; 31a+93b; 31a+94b; 31a+95b; 31a+96b; 31a+97b; 32a+1b; 32a+2b; 32a+3b; 32a+4b; 32a+5b; 32a+6b; 32a+7b; 32a+8b; 32a+9b; 32a+10b; 32a+11b; 32a+12b; 32a+13b; 32a+14b; 32a+15b; 32a+16b; 32a+17b; 32a+18b; 32a+19b; 32a+20b; 32a+21b; 32a+22b; 32a+23b; 32a+24b; 32a+25b; 32a+26b; 32a+27b; 32a+28b; 32a+29b; 32a+30b; 32a+31b; 32a+32b; 32a+33b; 32a+34b; 32a+35b; 32a+36b; 32a+37b; 32a+38b; 32a+39b; 32a+40b; 32a+41b; 32a+42b; 32a+43b; 32a+44b; 32a+45b; 32a+46b; 32a+47b; 32a+48b; 32a+49b; 32a+50b; 32a+51b; 32a+52b; 32a+53b; 32a+54b; 32a+55b; 32a+55b; 32a+57b; 32a+58b; 32a+59b; 32a+60b; 32a+61b; 32a+62b; 32a+63b; 32a+64b; 32a+65b; 32a+66b; 32a+67b; 32a+68b; 32a+69b; 32a+70b; 32a+71b; 32a+72b; 32a+73b; 32a+74b; 32a+75b; 32a+76b; 32a+77b; 32a+78b; 32a+79b; 32a+80b; 32a+81b; 32a+82b; 32a+83b; 32a+84b; 32a+85b; 32a+86b; 32a+87b; 32a+88b; 32a+89b; 32a+90b; 32a+91b; 32a+92b; 32a+93b; 32a+94b; 32a+95b; 32a+96b; 32a+97b; 33a+1b; 33a+2b; 33a+3b; 33a+4b; 33a+5b; 33a+6b; 33a+7b; 33a+8b; 33a+9b; 33a+10b; 33a+11b; 33a+12b; 33a+13b; 33a+14b; 33a+15b; 33a+16b; 33a+17b; 33a+18b; 33a+19b; 33a+20b; 33a+21b; 33a+22b; 33a+23b; 33a+24b; 33a+25b; 33a+26b; 33a+27b; 33a+28b; 33a+29b; 33a+30b; 33a+31b; 33a+32b; 33a+33b; 33a+34b; 33a+35b; 33a+36b; 33a+37b; 33a+38b; 33a+39b; 33a+40b; 33a+41b; 33a+42b; 33a+43b; 33a+44b; 33a+45b; 33a+46b; 33a+47b; 33a+48b; 33a+49b; 33a+50b; 33a+51b; 33a+52b; 33a+53b; 33a+54b; 33a+55b; 33a+55b; 33a+57b; 33a+58b; 33a+59b; 33a+60b; 33a+61b; 33a+62b; 33a+63b; 33a+64b; 33a+65b; 33a+66b; 33a+67b; 33a+68b; 33a+69b; 33a+70b; 33a+71b; 33a+72b; 33a+73b; 33a+74b; 33a+75b; 33a+76b; 33a+77b; 33a+78b; 33a+79b; 33a+80b; 33a+81b; 33a+82b; 33a+83b; 33a+84b; 33a+85b; 33a+86b; 33a+87b; 33a+88b; 33a+89b; 33a+90b; 33a+91b; 33a+92b; 33a+93b; 33a+94b; 33a+95b; 33a+96b; 33a+97b; 34a+1b; 34a+2b; 34a+3b; 34a+4b; 34a+5b; 34a+6b; 34a+7b; 34a+8b; 34a+9b; 34a+10b; 34a+11b; 34a+12b; 34a+13b; 34a+14b; 34a+15b; 34a+16b; 34a+17b; 34a+18b; 34a+19b; 34a+20b; 34a+21b; 34a+22b; 34a+23b; 34a+24b; 34a+25b; 34a+26b; 34a+27b; 34a+28b; 34a+29b; 34a+30b; 34a+31b; 34a+32b; 34a+33b; 34a+34b; 34a+35b; 34a+36b; 34a+37b; 34a+38b; 34a+39b; 34a+40b; 34a+41b; 34a+42b; 34a+43b; 34a+44b; 34a+45b; 34a+46b; 34a+47b; 34a+48b; 34a+49b; 34a+50b; 34a+51b; 34a+52b; 34a+53b; 34a+54b; 34a+55b; 34a+55b; 34a+57b; 34a+58b; 34a+59b; 34a+60b; 34a+61b; 34a+62b; 34a+63b; 34a+64b; 34a+65b; 34a+66b; 34a+67b; 34a+68b; 34a+69b; 34a+70b; 34a+71b; 34a+72b; 34a+73b; 34a+74b; 34a+75b; 34a+76b; 34a+77b; 34a+78b; 34a+79b; 34a+80b; 34a+81b; 34a+82b; 34a+83b; 34a+84b; 34a+85b; 34a+86b; 34a+87b; 34a+88b; 34a+89b; 34a+90b; 34a+91b; 34a+92b; 34a+93b; 34a+94b; 34a+95b; 34a+96b; 34a+97b; 35a+1b; 35a+2b; 35a+3b; 35a+4b; 35a+5b; 35a+6b; 35a+7b; 35a+8b; 35a+9b; 35a+10b; 35a+11b; 35a+12b; 35a+13b; 35a+14b; 35a+15b; 35a+16b; 35a+17b; 35a+18b; 35a+19b; 35a+20b; 35a+21b; 35a+22b; 35a+23b; 35a+24b; 35a+25b; 35a+26b; 35a+27b; 35a+28b; 35a+29b; 35a+30b; 35a+31b; 35a+32b; 35a+33b; 35a+34b; 35a+35b; 35a+36b; 35a+37b; 35a+38b; 35a+39b; 35a+40b; 35a+41b; 35a+42b; 35a+43b; 35a+44b; 35a+45b; 35a+46b; 35a+47b; 35a+48b; 35a+49b; 35a+50b; 35a+51b; 35a+52b; 35a+53b; 35a+54b; 35a+55b; 35a+55b; 35a+57b; 35a+58b; 35a+59b; 35a+60b; 35a+61b; 35a+62b; 35a+63b; 35a+64b; 35a+65b; 35a+66b; 35a+67b; 35a+68b; 35a+69b; 35a+70b; 35a+71b; 35a+72b; 35a+73b; 35a+74b; 35a+75b; 35a+76b; 35a+77b; 35a+78b; 35a+79b; 35a+80b; 35a+81b; 35a+82b; 35a+83b; 35a+84b; 35a+85b; 35a+86b; 35a+87b; 35a+88b; 35a+89b; 35a+90b; 35a+91b; 35a+92b; 35a+93b; 35a+94b; 35a+95b; 35a+96b; 35a+97b; 36a+1b; 36a+2b; 36a+3b; 36a+4b; 36a+5b; 36a+6b; 36a+7b; 36a+8b; 36a+9b; 36a+10b; 36a+11b; 36a+12b; 36a+13b; 36a+14b; 36a+15b; 36a+16b; 36a+17b; 36a+18b; 36a+19b; 36a+20b; 36a+21b; 36a+22b; 36a+23b; 36a+24b; 36a+25b; 36a+26b; 36a+27b; 36a+28b; 36a+29b; 36a+30b; 36a+31b; 36a+32b; 36a+33b; 36a+34b; 36a+35b; 36a+36b; 36a+37b; 36a+38b; 36a+39b; 36a+40b; 36a+41b; 36a+42b; 36a+43b; 36a+44b; 36a+45b; 36a+46b; 36a+47b; 36a+48b; 36a+49b; 36a+50b; 36a+51b; 36a+52b; 36a+53b; 36a+54b; 36a+55b; 36a+55b; 36a+57b; 36a+58b; 36a+59b; 36a+60b; 36a+61b; 36a+62b; 36a+63b; 36a+64b; 36a+65b; 36a+66b; 36a+67b; 36a+68b; 36a+69b; 36a+70b; 36a+71b; 36a+72b; 36a+73b; 36a+74b; 36a+75b; 36a+76b; 36a+77b; 36a+78b; 36a+79b; 36a+80b; 36a+81b; 36a+82b; 36a+83b; 36a+84b; 36a+85b; 36a+86b; 36a+87b; 36a+88b; 36a+89b; 36a+90b; 36a+91b; 36a+92b; 36a+93b; 36a+94b; 36a+95b; 36a+96b; 36a+97b; 37a+1b; 37a+2b; 37a+3b; 37a+4b; 37a+5b; 37a+6b; 37a+7b; 37a+8b; 37a+9b; 37a+10b; 37a+11b; 37a+12b; 37a+13b; 37a+14b; 37a+15b; 37a+16b; 37a+17b; 37a+18b; 37a+19b; 37a+20b; 37a+21b; 37a+22b; 37a+23b; 37a+24b; 37a+25b; 37a+26b; 37a+27b; 37a+28b; 37a+29b; 37a+30b; 37a+31b; 37a+32b; 37a+33b; 37a+34b; 37a+35b; 37a+36b; 37a+37b; 37a+38b; 37a+39b; 37a+40b; 37a+41b; 37a+42b; 37a+43b; 37a+44b; 37a+45b; 37a+46b; 37a+47b; 37a+48b; 37a+49b; 37a+50b; 37a+51b; 37a+52b; 37a+53b; 37a+54b; 37a+55b; 37a+55b; 37a+57b; 37a+58b; 37a+59b; 37a+60b; 37a+61b; 37a+62b; 37a+63b; 37a+64b; 37a+65b; 37a+66b; 37a+67b; 37a+68b; 37a+69b; 37a+70b; 37a+71b; 37a+72b; 37a+73b; 37a+74b; 37a+75b; 37a+76b; 37a+77b; 37a+78b; 37a+79b; 37a+80b; 37a+81b; 37a+82b; 37a+83b; 37a+84b; 37a+85b; 37a+86b; 37a+87b; 37a+88b; 37a+89b; 37a+90b; 37a+91b; 37a+92b; 37a+93b; 37a+94b; 37a+95b; 37a+96b; 37a+97b; 38a+1b; 38a+2b; 38a+3b; 38a+4b; 38a+5b; 38a+6b; 38a+7b; 38a+8b; 38a+9b; 38a+10b; 38a+11b; 38a+12b; 38a+13b; 38a+14b; 38a+15b; 38a+16b; 38a+17b; 38a+18b; 38a+19b; 38a+20b; 38a+21b; 38a+22b; 38a+23b; 38a+24b; 38a+25b; 38a+26b; 38a+27b; 38a+28b; 38a+29b; 38a+30b; 38a+31b; 38a+32b; 38a+33b; 38a+34b; 38a+35b; 38a+36b; 38a+37b; 38a+38b; 38a+39b; 38a+40b; 38a+41b; 38a+42b; 38a+43b; 38a+44b; 38a+45b; 38a+46b; 38a+47b; 38a+48b; 38a+49b; 38a+50b; 38a+51b; 38a+52b; 38a+53b; 38a+54b; 38a+55b; 38a+55b; 38a+57b; 38a+58b; 38a+59b; 38a+60b; 38a+61b; 38a+62b; 38a+63b; 38a+64b; 38a+65b; 38a+66b; 38a+67b; 38a+68b; 38a+69b; 38a+70b; 38a+71b; 38a+72b; 38a+73b; 38a+74b; 38a+75b; 38a+76b; 38a+77b; 38a+78b; 38a+79b; 38a+80b; 38a+81b; 38a+82b; 38a+83b; 38a+84b; 38a+85b; 38a+86b; 38a+87b; 38a+88b; 38a+89b; 38a+90b; 38a+91b; 38a+92b; 38a+93b; 38a+94b; 38a+95b; 38a+96b; 38a+97b; 39a+1b; 39a+2b; 39a+3b; 39a+4b; 39a+5b; 39a+6b; 39a+7b; 39a+8b; 39a+9b; 39a+10b; 39a+11b; 39a+12b; 39a+13b; 39a+14b; 39a+15b; 39a+16b; 39a+17b; 39a+18b; 39a+19b; 39a+20b; 39a+21b; 39a+22b; 39a+23b; 39a+24b; 39a+25b; 39a+26b; 39a+27b; 39a+28b; 39a+29b; 39a+30b; 39a+31b; 39a+32b; 39a+33b; 39a+34b; 39a+35b; 39a+36b; 39a+37b; 39a+38b; 39a+39b; 39a+40b; 39a+41b; 39a+42b; 39a+43b; 39a+44b; 39a+45b; 39a+46b; 39a+47b; 39a+48b; 39a+49b; 39a+50b; 39a+51b; 39a+52b; 39a+53b; 39a+54b; 39a+55b; 39a+55b; 39a+57b; 39a+58b; 39a+59b; 39a+60b; 39a+61b; 39a+62b; 39a+63b; 39a+64b; 39a+65b; 39a+66b; 39a+67b; 39a+68b; 39a+69b; 39a+70b; 39a+71b; 39a+72b; 39a+73b; 39a+74b; 39a+75b; 39a+76b; 39a+77b; 39a+78b; 39a+79b; 39a+80b; 39a+81b; 39a+82b; 39a+83b; 39a+84b; 39a+85b; 39a+86b; 39a+87b; 39a+88b; 39a+89b; 39a+90b; 39a+91b; 39a+92b; 39a+93b; 39a+94b; 39a+95b; 39a+96b; and 39a+97b.

Although the above therapeutic agents (e.g., individual components of anti-scarring drug combinations, secondary agents, other components in the compositions according to the present invention) have been provided for the purposes of illustration, it should be understood that the present invention is not so limited. For example, although agents are specifically referred to above, the present invention should be understood to include analogues, derivatives, pharmaceutically active or acceptable salts, metabolites, and conjugates of such agents. In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially. However, for the individual components of anti-scarring combinations, if they are delivered sequentially, there have to be a time during which they are all present as a composition after delivery.

The following examples are offered by way of illustration, and not by way of limitation.

### EXAMPLES

### Example 1

### Parylene Coating

A metallic portion of an electrical device (e.g., a neurostimulator or an electrical lead) is washed by dipping it into HPLC grade isopropanol. A parylene primer layer (about 1 to 10 μm) is deposited onto the cleaned electrical device using a parylene coater (e.g., PDS 2010 LABCOATER 2 from Cookson Electronics) and di-p-xylylene (PARYLENE N) or dichloro-di-p-xylylene (PARYLENE D) (both available from Specialty Coating Systems, Indianapolis, Ind.) as the coating feed material.

### Example 2

### Drug Combination Coating—Partial Coating

Drug combinations (amoxapine and prednisolone) are prepared by adding amoxapine and prednisolone (5 mg, 10 mg, 50 mg, 100 mg, 200 mg and 500 mg) in 5 ml HPLC grade tetrahydrofuran (THF). A coated portion of a parylene-coated device (as prepared in, e.g., Example 1) is dipped into the combination of amoxapine and prednisolone in THF. After a selected incubation time, the device is removed from the solution and dried in a forced air oven (50° C.). The device then is further dried in a vacuum oven overnight. The amount of amoxapine and prednisolone used in each solution and the incubation time is varied such that the amount of each individual component (amoxapine and prednisolone) coated onto the device is in the range of 0.06 μg/mm² to 10 μg/mm² (μg each of amoxapine and prednisolone coated/mm² of the device after the device has been dip coated in the solution of amoxapine and prednisolone in THF). The time during which the device is maintained in the solution of amoxapine and prednisolone may be varied, where longer soak times generally provide for more amoxapine and prednisolone to be adsorbed onto the device.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 3

### Drug Combination Coating—Complete Coating

Drug combinations (amoxapine and prednisolone) are prepared by dissolving amoxapine and prednisolone (5 mg, 10 mg, 50 mg, 100 mg, 200 mg and 500 mg) in 5 ml HPLC grade THF. An entire parylene coated device (coated as in, e.g., Example 1) is then dipped into the combination of amoxapine and prednisolone in THF. After a selected incubation time, the device is removed and dried in a forced air oven (50° C.). The device is then further dried in a vacuum oven overnight. The amount of amoxapine and prednisolone used in each solution and the incubation time is varied such that the amount of each component (amoxapine and prednisolone) coated onto the device is in the range of 0.06 μg/mm² to 100 μg/mm².

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 4

### Application of a Parylene Overcoat

A device coated with a combination of amoxapine and prednisolone (prepared as in, e.g., Example 2 or 3) is placed in a parylene coater and an additional thin layer of parylene is deposited on the amoxapine and prednisolone coated device using the procedure described in Example 1. The coating duration is selected to provide a parylene top coat thickness that will cause the device to have a desired elution profile for the drug combination of amoxapine and prednisolone.

### Example 5

### Application of an Echogenic Coating Layer

DESMODUR (an isocyanate pre-polymer Bayer AG) (25% w/v) is dissolved in a 50:50 mixture of dimethylsulfoxide and tetrahydrofuran. A device coated with amoxapine and prednisolone, then overcoated with parylene (prepared as in, e.g., Example 4), is then dipped into the pre-polymer solution. The device is removed from the solution after a selected incubation time, and the coating is then partially dried at room temperature for 3 to 5 minutes. The device is then immersed in a beaker of water (room temperature) for 3-5 minutes to cause the polymerization reaction to occur rapidly. An echogenic coating is formed.

### Example 6

### Drug Combination/Polymer Coating—Partial Coating

Several 5% solutions of poly(ethylene-co-vinyl acetate) {EVA} (60% vinyl acetate) are prepared using THF as the solvent. Selected amounts of a drug combination of amoxapine and prednisolone (0.01%, 0.05%, 0.1%, 0.5%, 1%, 5%, 10%, 20%, 30% (w/w drug to polymer) are added to the EVA solutions. An electrical device or a portion thereof is dipped into a (amoxapine and prednisolone)/EVA solution. After removing the device from the solution, the coating is dried by placing the device in a forced air oven (40° C.) for 3 hours. The coated device is then further dried under vacuum for 24 hours. This dip coating process may be repeated to increase the amount of polymer/(amoxapine and prednisolone) coated onto the device. In addition, higher amoxapine and prednisolone concentrations in the polymer/THF/(amoxapine and prednisolone) solution and/or a longer soak time may be used to increase the amount of polymer/(amoxapine and prednisolone) that is coated onto the device.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 7

### Drug Combination—Heparin Coating

Several 5% solutions of poly(ethylene-co-vinyl acetate) {EVA} (60% vinyl acetate) are prepared using THF as the solvent. Selected amounts (0.01%, 0.05%, 0.1%, 0.5%, 1%, 5%, 10%, 20%, 30% (w/w drug to polymer)) of drug combination (amoxapine and prednisolone) and a solution of tridodecyl methyl ammonium chloride-heparin complex (PolySciences) are added to each of the EVA solutions. All or a portion of an electrical device is dipped into the (amoxapine and prednisolone)/EVA solution. After removing the device from the solution, the coating is dried by placing the device in a forced air oven (40° C.) for 3 hours. The coated device is then further dried under vacuum for 24 hours. The dip coating process may be repeated to increase the amount of polymer/heparin/(amoxapine and prednisolone) complex coated onto the device.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 8

### Drug Combination—Heparin/Heparin Coating

An uncoated portion of a drug combination (amoxapine and prednisolone)-heparin coated device (prepared as in, e.g., Example 7) is dipped into a 5% EVA/THF solution containing a selected amount of a tridodecyl methyl ammonium chloride-heparin complex solution (PolySciences) (0.1%, 0.5%, 1%, 2.5%, 5%, 10% (v/v)). After removing the device from the solution, the coating is dried by placing the device in a forced air oven (40° C.) for 3 hours. The coated device is then further dried under vacuum for 24 hours. This provides a device with a (amoxapine and prednisolone)/heparin coating on one or more portions of the device and a heparin coating on one or more other parts of the device.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 9

### Drug Combination/Polymer Coating—Partial Coating

Several 5% solutions of poly(styrene-co-isobutylene-styrene) (SIBS) are prepared using THF as the solvent. A selected amount (0.01%, 0.05%, 0.1%, 0.5%, 1%, 5%, 10%, 20%, 30% (w/w drug to polymer)) of drug combination (amoxapine and prednisolone) is added to each SIBS solution. One or more portions of a device are dipped into the (amoxapine and prednisolone)/SIBS solution. After removing the device from the solution, the coating is dried by placing the device in a forced air oven (40° C.) for 3 hours. The coated device is then further dried under vacuum for 24 hours. The dip coating process may be repeated to increase the amount of polymer/(amoxapine and prednisolone) coated onto the device. In addition, higher amoxapine and prednisolone concentrations in the polymer/THF/(amoxapine and prednisolone) solution and/or a longer soak time may be used to increase the amount of polymer/(amoxapine and prednisolone) that is coated onto the device.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 10

### Drug Combination/Polymer Coating—Echogenic Overcoat

A drug combination (amoxapine and prednisolone)-coated electrical device prepared as in Example 9 is dipped into a DESMODUR solution (50% w/v) (50:50 mixture of dimethylsulfoxide and tetrahydrofuran). The device is then removed and the coating is partially dried at room temperature for 3 to 5 minutes. The device is then immersed in a beaker of water (room temperature) for 3-5 minutes to cause the polymerization reaction to occur rapidly. An echogenic coating is thereby formed.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 11

### Polymer/Echogenic Coating

A 5% solution of poly(styrene-co-isobutylene-styrene) (SIBS) is prepared using THF as the solvent. An electrical device is dipped into the SIBS solution. After a selected incubation time, the device is removed from the solution, and the coating is dried by placing the device in a forced air oven (40° C.) for 3 hours. The coated device is then further dried under vacuum for 24 hours.

A coated device is dipped into a DESMODUR solution (50:50 mixture of dimethylsulfoxide and tetrahydrofuran). The device is then removed and the coating is then partially dried at room temperature for 3 to 5 minutes. The device is then immersed in a beaker of water (room temperature) for 3-5 minutes to cause the polymerization reaction to occur rapidly. The device is dried under vacuum for 24 hours at room temperature. All or a portion of the coated device is immersed into a solution of a drug combination (amoxapine and prednisolone) (5% w/v in methanol). The device is removed and dried at 40° C. for 1 hour and then under vacuum for 24 hours.

The amount of the drug combination (amoxapine and prednisolone) absorbed by the polymeric coating can be altered by changing the concentration of the solution of amoxapine and prednisolone, the immersion time, and/or the solvent composition of the solution of amoxapine and prednisolone.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 12

### Drug Combination/Siloxane Coating—Partial Coating

An electrical device is coated with a silioxane layer by exposing the device to gaseous tetramethylcyclotetrasiloxane that is then polymerized by low energy plasma polymerization onto the device surface. The thickness of the siloxane layer can be increased by increasing the polymerization time. After polymerization, a portion of the coated device is then immersed into a solution of a drug combination (amoxapine and prednisolone) (5% w/v) in THF solution for a selected period of time to allow the drug combination of amoxapine and prednisolone to absorb into the siloxane coating. The device is then removed from the solution and is dried for 2 hours at 40° C. in a forced air oven. The device is then further dried under vacuum at room temperature for 24 hours. The amount of drug combination of amoxapine and prednisolone coated onto the device can be varied by altering the concentration of the drug combination (amoxapine and prednisolone)/THF solution and by altering the immersion time of the device in the drug combination (amoxapine and prednisolone)/THF solution.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 13

### Spray-Coated Devices

Several 2% solutions of poly(styrene-co-isobutylene-styrene) (SIBS) (50 ml) are prepared using THF as the solvent. A selected amount of a drug combination (amoxapine and prednisolone) (0.01%, 0.05%, 0.1%, 0.5%, 1%, 2.5%, 5%, 10% and 20% (w/w with respect to the polymer)) is added to each solution. An electrical device is held with a pair of tweezers and is then spray coated with one of the drug combination (amoxapine and prednisolone)/polymer solutions using an airbrush. The device is then air-dried. The device is then held in a new location using the tweezers and a second coat of a drug combination (amoxapine and prednisolone)/polymer solution having the same concentration is applied to the device. The device is air-dried and is then dried under vacuum at room temperature overnight. The total amount of drug combination (amoxapine and prednisolone) coated onto the device can be altered by changing the amoxapine and prednisolone content in the solution as well as by increasing the number of coatings that are applied.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 14

### Drug Coated Device—Non-Degradable

An electrical device is attached to a rotating mandrel. A solution of a drug combination of amoxapine and prednisolone (5% w/w) in a polyurethane (CHRONOFLEX 85A; CardioTech Biomaterials)/THF solution (2.5% w/v) is then sprayed onto all or a portion of the outer surface of the device. The solution is sprayed on at a rate that ensures that the device is not damaged or saturated with the sprayed solution. The device is allowed to air dry after which it is dried under vacuum for 24 hours.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 15

### Drug Coated Device—Degradable

An electrical device is attached to a rotating mandrel. A solution of a drug combination or amoxapine and prednisolone (5% w/w) in PLGA/ethyl acetate (2.5% w/v) is then sprayed onto all or portion of the outer surface of the device. The solution is sprayed on at a rate that ensures that the device is not damaged or saturated with the sprayed solution. The device is allowed to air dry, after which it is dried under vacuum at room temperature for 24 hours.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 16

### Drug Coated Device—Degradable Overcoat

A drug-coated electrical device prepared as in Example 14 or Example 15 is attached to a rotating mandrel. A PLGA/ethyl acetate solution (2.5% w/v) is then sprayed onto all or a portion of the outer surface of the device, such that a coating is formed over the first drug containing coating. The solution is sprayed on at a rate that ensures that the device is not damaged or saturated with the sprayed solution. The device is allowed to air dry after which it is dried under vacuum at room temperature for 24 hours.

### Example 17

### Drug Combination—Loaded Microsphere Formulation

A drug combination of amoxapine and prednisolone (10% w/w) is added to a solution of PLGA (50/50, Mw≈54,000) in DCM (5% w/v). The solution is vortexed and then poured into a stirred (overhead stirrer with a 3 bladed TEFLON coated stirrer) aqueous PVA solution (approx. 89% hydrolysed, Mw≈13,000, 2% w/v). The solution is stirred for 6 hours after which the solution is centrifuged to sediment the microspheres. The microspheres are resuspended in water. The centrifugation process is repeated 4 times. The final microsphere solution is flash frozen in an acetone/dry-ice bath. The frozen solution is then freeze-dried to produce a fine powder. The size of the microspheres formed can be altered by changing the stirring speed and/or the PVA solution concentration. The freeze-dried powder can be resuspended in PBS or saline and can be used for direct injection, as an incubation fluid or as an irrigation fluid.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 18

### Drug Combination—Coated Device (Exterior Coating)

All or a portion of an electrical device is dipped into a polyurethane (CHRONOFLEX 85A)/THF solution (2.5% w/v). The coated device is allowed to air dry for 10 seconds. The device is then rolled in a powdered drug combination of amoxapine and prednisolone that has been spread thinly on a piece of release liner to provide a device coated with between 0.1 to 10 mg of the drug combination of amoxapine and prednisolone. The rolling process is done in such a manner that the drug combination (amoxapine and prednisolone) powder predominantly adheres to the exterior side of the coated device. The device is air-dried for 1 hour followed by vacuum drying at room temperature for 24 hours.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 19

### Drug Combination—Coated Device (Exterior Coating) with a Heparin Coating

A drug combination-coated device prepared as in Example 18 is further coated with a heparin coating. A device prepared as in Example 18 is dipped into a solution of heparin-benzalkonium chloride complex (1.5% (w/v) in isopropanol, STS Biopolymers). The device is removed from the solution and air-dried for 1 hour followed by vacuum drying for 24 hours. This process coats both the interior and exterior surfaces of the device with heparin.

### Example 20

### Partial Drug Combination Coating of a Device

An electrical device is attached to a rotating mandrel. A mask system is set up so that only a portion of the device surface is exposed. The drug combination of amoxapine and prednisolone (5% w/w) in a polyurethane (CHRONOFLEX 85A)/THF solution (2.5% w/v) is then sprayed onto the exposed portion of the device. The solution is sprayed on at a rate that ensures that the device is not damaged or saturated with the sprayed solution. The device is allowed to air dry after which it is dried under vacuum at room temperature for 24 hours.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 21

### Drug Combination—Dexamethasone Coated Device

An electrical device is coated as in Example 20. The mask is then rearranged so that a previously masked portion of the device is exposed. The exposed portion of the device is then sprayed with a dexamethasone (10% w/w)/polyurethane (CHRONOFLEX 85A)/THF solution (2.5% w/v). The device is air dried, after which it is dried under vacuum at room temperature for 24 hours.

In addition to the drug combination of amoxapine and prednisolone and the drug dexamethasone, further exemplary drug combinations, or individual components thereof, that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 22

### Drug Combination—Heparin Coated Device

An electrical device is coated as in Example 20. The mask is then rearranged so that only a previously masked portion of the device is exposed. The exposed surface of the device is then sprayed with a heparin-benzalkonium chloride complex (1.5% (w/v) in isopropanol (STS Biopolymers). The sample is air dried after which it is dried under vacuum for 24 hours.

In addition to the drug combination of amoxapine and prednisolone, further exemplary drug combinations that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 23

### Drug Combination—Dexamethaxone Coated Device

An electrical device is attached to a rotating mandrel. A solution of a drug combination of amoxapine and prednisolone (5% w/w) and dexamethazone (5% w/w) in a PLGA (50/50, Mw≈54,000)/ethyl acetate solution (2.5% w/v) is sprayed onto all or a portion of the device. The solution is sprayed on at a rate that ensures that the device is not damaged or saturated with the sprayed solution. The device is allowed to air dry after which it is dried under vacuum at room temperature for 24 hours.

In addition to the drug combination of amoxapine and prednisolone and the drug dexamethasone, further exemplary drug combinations, or individual components thereof, that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 24

### Drug Combination—Dexamethasone Coated Device (Sequential Coating)

An electrical device is attached to a rotating mandrel. A solution of a drug combination of amoxapine and prednisolone (5% w/w) in a PLGA (50/50, Mw≈54,000)]ethyl acetate solution (2.5% w/v) is sprayed onto the outer surface of the device. The solution is sprayed on at a rate that ensures that the device is not damaged or saturated with the sprayed solution. The device is allowed to air dry. A methanol solution of dexamethasone (2% w/v) is then sprayed onto the outer surface of the device (at a rate that ensures that the device is not damaged or saturated with the sprayed solution). The device is allowed to air dry, after which it is dried under vacuum at room temperature for 24 hours.

In addition to the drug combination of amoxapine and prednisolone and the drug dexamethasone, further exemplary drug combinations, or individual components thereof, that may be used to coat the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 25

### Drug Combination—Loading an Electrical Lead Comprising a Porous Electrode—Amoxapine and Prednisolone

10 ml combinations of a drug combination (amoxapine and prednisolone) are prepared by weighing 1 mg, 5 mg, 10 mg, 20 mg, 50 mg, 75 mg, 100 mg, 200 mg, and 500 mg of the drug combination into 20 ml glass scintillation vials, respectively, and then adding HPLC grade acetone. The solutions are gently shaken on an orbital shaker for 1 hour at room temperature. An electrical pacing lead that comprises a porous ball shaped electrode tip (Medtronic, Inc.) is placed on a bench and a glass microscope slide is placed under the tip portion of the lead. Using a 200 μl pipettor (Gilson) with the pipette tip touching the electrode tip, the 0.1 mg/ml drug combination (amoxapine and prednisolone) solution is slowly applied to the porous electrode tip until the electrode tip does not absorb any more solution. The electrode is then allowed to air dry for 6 hours. The process is repeated for all the prepared solutions of amoxapine and prednisolone using a fresh electrode for each solution.

In addition to the drug combination of amoxapine and prednisolone, further exemplary drug combinations that may be used to load the electrode tip of the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 26

### Drug Combination—Loading an Electrical Lead Comprising a Porous Electrode—Drug Combination/Beclomethasone

Several saturated 10 ml acetone solutions of beclomethasone diproprionate anhydrous are prepared by adding the beclomethasone diproprionate anhydrous to 10 ml acetone in 20 ml glass scintillation vials until no more beclomethasone diproprionate anhydrous will dissolve and solid beclomethasone diproprionate anhydrous remains at the bottom of the vial. To each of these saturated solutions, 1 mg, 5 mg, 10 mg, 20 mg, 50 mg, 75 mg, 100 mg, 200 mg, and 500 mg of a drug combination of amoxapine and prednisolone are added respectively. The solutions are gently shaken on an orbital shaker for 1 hour at room temperature. An electrical pacing lead that comprises a porous ball shaped electrode tip (Medtronic, Inc.) is placed on a bench and a glass microscope slide is placed under the tip portion of the lead. Using a 200 μL Gilson pipettor with the pipette tip touching the electrode tip, the 0.1 mg/ml drug combination (amoxapine and prednisolone) solution is slowly applied to the porous electrode tip until the electrode tip will not absorb any more solution. The electrode is then allowed to air dry for 6 hour. The process is repeated for all the prepared solutions of amoxapine and prednisolone using a fresh electrode for each solution.

In addition to the drug combination of amoxapine and prednisolone and the drug beclomethasone, further exemplary drug combinations, or individual components thereof, that may be used to load the electrode tip of the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 27

### Drug Combination—Loading an Electrical Lead Comprising a Porous Electrode—Drug Combination/Polymer

10 ml solutions of a drug combination of amoxapine and prednisolone are prepared by weighing 1 mg, 5 mg, 10 mg, 20 mg, 50 mg, 75 mg, 100 mg 200 mg and 500 mg of the drug combination (amoxapine and prednisolone) into 20 ml glass scintillation vials, respectively, and then adding HPLC grade tetrahydrofuran (THF). 1 g of a MePEG(2000)-PDLLA (60:40) diblock copolymer is added to each vial. The solutions are gently shaken on an orbital shaker for 6 hours at room temperature. An electrical pacing lead that comprises a porous ball shaped electrode tip (Medtronic, Inc.) is placed on a bench and a glass microscope slide is placed under the tip portion of the lead. Using a 200 μL Gilson pipettor with the pipette tip touching the electrode tip, the 0.1 mg/ml solution of amoxapine and prednisolone is slowly applied to the porous electrode tip until the electrode tip will not absorb any more solution. The electrode is then allowed to air dry for 6 hour. The process is repeated for all the prepared solutions of amoxapine and prednisolone using a fresh electrode for each solution.

In addition to the drug combination of amoxapine and prednisolone, further exemplary drug combinations that may be used to load the electrode tip of the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 28

### Drug Combination—Loading an Electrical Lead Comprising a Porous Electrode—Drug Combination/Beclomethasone/Polymer

Several saturated 10 ml acetone solutions of beclomethasone diproprionate anhydrous are prepared by adding the beclomethasone diproprionate anhydrous to 10 ml acetone in 20 ml glass scintillation vials until no more beclomethasone diproprionate anhydrous will dissolve and solid beclomethasone diproprionate anhydrous remains at the bottom of the vial. To each of these saturated solutions, 1 mg, 5 mg, 10 mg, 20 mg, 50 mg, 75 mg, 100 mg, 200 mg, and 500 mg of a drug combination of amoxapine and prednisolone are added, respectively. 1 g of a MePEG(2000)-PDLLA (60:40) diblock copolymer is added to each vial. The solutions are gently shaken on an orbital shaker for 6 hours at room temperature. An electrical pacing lead that comprises a porous ball shaped electrode tip (Medtronic) is placed on a bench and a glass microscope slide is placed under the tip portion of the lead. Using a 200 μL Gilson pipettor with the pipette tip touching the electrode tip, the 0.1 mg/ml solution of amoxapine and prednisolone is slowly applied to the porous electrode tip until the electrode tip will not absorb any more solution. The electrode is then allowed to air dry for 6 hour. The process is repeated for all the prepared solutions of amoxapine and prednisolone using a fresh electrode for each solution.

In addition to the drug combination of amoxapine and prednisolone and the drug beclomethasone, further exemplary drug combinations, or individual components thereof, that may be used to load the electrode tip of the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 29

### Drug Combination—Loading an Electrical Lead Comprising a Porous Electrode—Drug Combination Dipping

10 ml solutions of a drug combination of amoxapine and prednisolone are prepared by weighing 1 mg, 5 mg, 10 mg, 20 mg, 50 mg, 75 mg, 100 mg, 200 mg, and 500 mg of the drug combination (amoxapine and prednisolone) into 20 ml glass scintillation vials, respectively, and then adding HPLC grade acetone. The solutions are gently shaken on an orbital shaker for 1 hour at room temperature. The tip of an electrical pacing lead that comprises a porous ball shaped electrode tip (Medtronic, Inc.) is immersed to a depth of about 1 cm into the 0.1 mg/ml solution. After about 2 hours, the tip portion is removed from the solution and is allowed to air dry for 6 hour. The electrode is further dried under vacuum for 24 hours. The process is repeated for all the prepared solutions of amoxapine and prednisolone using a fresh electrode for each solution.

In addition to the drug combination of amoxapine and prednisolone, further exemplary drug combinations that may be used to load the electrode tip of the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 30

### Drug Combination—Loading an Electrical Lead Comprising a Porous Electrode—Drug Combination/Beclomethasone

Several saturated 10 ml acetone solutions of beclomethasone diproprionate anhydrous are prepared by adding the beclomethasone diproprionate anhydrous to 10 ml acetone in 20 ml glass scintillation vials until no more beclomethasone diproprionate anhydrous will dissolve and solid beclomethasone diproprionate anhydrous remains at the bottom of the vial. To each of these saturated solutions, 1 mg, 5 mg, 10 mg, 20 mg, 50 mg, 75 mg, 100 mg, 200 mg, and 500 mg of a drug combination of amoxapine and prednisolone are added respectively. The solutions are gently shaken on an orbital shaker for 1 hour at room temperature. The tip of an electrical pacing lead that comprises a porous ball shaped electrode tip (Medtronic) is immersed to a depth of about 1 cm into the 0.1 mg/ml solution. After about 2 hours, the tip portion is removed from the solution and is allowed to air dry for 6 hour. The electrode is further dried under vacuum for 24 hours. The process is repeated for all the prepared solutions of amoxapine and prednisolone using a fresh electrode for each solution.

In addition to the drug combination of amoxapine and prednisolone and the drug beclomethasone, further exemplary drug combinations, or individual components thereof, that may be used to load the electrode tip of the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 31

### Drug Combination—Loading a Screw-in Electrical Lead—Drug Combination Dipping

10 ml solutions of a drug combination of amoxapine and prednisolone are prepared by weighing 1 mg, 5 mg, 10 mg, 20 mg, 50 mg, 75 mg, 100 mg, 200 mg, and 500 mg of the drug combination (amoxapine and prednisolone) into 20 ml glass scintillation vials, respectively, and then adding HPLC grade methanol. The solutions are gently shaken on an orbital shaker for 1 hour at room temperature. The tip of an electrical pacing lead that comprises a screw in electrode tip (e.g., CAPSUREFIX NOVUS 5076, Medtronic, Inc.) is immersed to a depth of about 1 cm into the 0.1 mg/ml solution. After about 2 hours, the tip portion is removed from the solution and is allowed to air dry for 6 hour. The electrode is further dried under vacuum for 24 hours. The process is repeated for all the prepared solutions of amoxapine and prednisolone using a fresh electrode for each solution.

In addition to the drug combination of amoxapine and prednisolone, further exemplary drug combinations that may be used to load the electrode tip of the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 32

### Drug Combination—Loading a Screw-in Electrical Lead—Drug Combination/Polymer Dipping

A polyurethane solution (CHRONOFLEX AL 85 A) is prepared by dissolving 20 g of the polyurethane in 200 ml tetrahydrofuran (THF). 10 ml aliquots of this solution are placed in 20 ml glass scintillation vials. 1 mg, 5 mg, 10 mg, 20 mg, 50 mg, 75 mg, 100 mg, 200 mg, and 500 mg of a drug combination of amoxapine and prednisolone are then added to each of the vials, respectively. The solutions are tumbled for 3 hours at 20 rpm. The tip of an electrical pacing lead that comprises a screw in electrode tip (e.g., CAPSUREFIX NOVUS 5076) is immersed to a depth of about 1 cm into the 0.1 mg/ml solution of amoxapine and prednisolone and then it is slowly withdrawn from the solution. The coated portion is allowed to air dry for 10 min. The screw-in portion of the electrode is then immersed in a solution of HPLC grade THF. After 1 hour the screw-in portion of the electrode is removed from the THF solution and is immersed in a fresh THF solution for 30 min. The electrode is then removed from the THF solution and is allowed to air dry for 2 hour. The electrode is further dried under vacuum for 24 hours. The process is repeated for all the prepared solutions of amoxapine and prednisolone using a fresh electrode for each solution.

In addition to the drug combination of amoxapine and prednisolone, further exemplary drug combinations that may be used to load the electrode lead of the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 33

### Drug-Loading a Screw-in Electrical Lead—Halofuginone/Polymer Spraying

A polyurethane solution (CHRONOFLEX AL 85 A) is prepared by dissolving 15 g of the polyurethane in 200 ml tetrahydrofuran (THF). 10 ml aliquots of this solution are placed in 20 ml glass scintillation vials. 1 mg, 5 mg, 10 mg, 20 mg, 50 mg, 75 mg, 100 mg, 200 mg, and 500 mg halofuginone are then added to each of the vials respectively. The solutions are tumbled for 3 hours at 20 rpm. The tip of an electrical pacing lead that comprises a screw in electrode tip (e.g., CAPSUREFIX NOVUS 5076) is screwed into the end of a silastic rod until the screw-in portion is completely incorporated into the silastic rod. The silastic rod it the attached to an overhead stirrer and the stir speed is set at 40 rpm. The 0.1 mg/ml halofuginone solution is placed in a 3 ml glass syringe that is then attached to an ultrasonic spray head (Sonus, Inc). The syringe is placed in a syringe pump. The solution is then sprayed onto the tip portion of the lead at a flow rate of 0.5 ml/min. Once the electrical lead tip is evenly coated with a halofuginone/polymer solution, the spraying is stopped and the coating is allowed to air dry for 1 hour. The electrode is unscrewed from the silastic rod. The electrode is further dried under vacuum for 24 hours. The process is repeated for all the prepared halofuginone solutions using a fresh electrode each time.

Exemplary drug combinations that may be used to load the device include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 34

### Drug Combination—Loading an Electrode Annular Shaped Monolithic Controlled Release Device—Amoxapine and Prednisolone

10 ml solutions of a drug combination of amoxapine and prednisolone are prepared by weighing 1 mg, 5 mg, 10 mg, 20 mg, 50 mg, 75 mg, 100 mg, 200 mg, and 500 mg of the drug combination (amoxapine and prednisolone) into 20 ml glass scintillation vials, respectively and then adding HPLC grade methanol. The solutions are gently shaken on an orbital shaker for 1 hour at room temperature. A silicone rubber annular shaped monolithic controlled release device used in the construction of a CAPSURE Z lead (Model 5534, Medtronic, Inc), is immersed in the 0.1 mg/ml solution of amoxapine and prednisolone for 3 hours. Using a pair of tweezers, the silicone rubber piece is removed from the solution, gently shaken to remove the excess solution and is then air-dried for 5 hour. The air-dried component is then dried under vacuum for 24 hours. The drug loaded silicone rubber component is then used in the assembly of the lead.

In addition to the drug combination of amoxapine and prednisolone, further exemplary drug combinations that may be used to load the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 35

### Drug Combination—Loading an Electrode Annular Shaped Monolithic Controlled Release Device—Drug Combination/Dexamethasone

Several saturated 10 ml methanol solutions of dexamethasone are prepared by adding the dexamethasone to 10 ml methanol in 20 ml glass scintillation vials until no more dexamethasone will dissolve and solid dexamethasone remains at the bottom of the vial. To each of these saturated solutions, 1 mg, 5 mg, 10 mg, 20 mg, 50 mg, 75 mg, 100 mg, 200 mg, and 500 mg of a drug combination of amoxapine and prednisolone are added, respectively. The solutions are gently shaken on an orbital shaker for 1 hour at room temperature. A silicone rubber annular shaped monolithic controlled release device used in the construction of a CAPSURE Z lead (Medtronic, Inc) is immersed in the 0.1 mg/ml solution of amoxapine and prednisolone for 3 hours. Using a pair of tweezers, the silicone rubber piece is removed from the solution, gently shaken to remove the excess solution and is then air-dried for 5 hour. The air-dried component is then dried under vacuum for 24 hours. The drug loaded silicone rubber component is then used in the assembly of the lead.

In addition to the drug combination of amoxapine and prednisolone and the drug dexamethsone, further exemplary drug combinations, or individual components thereof, that may be used to load the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 36

### Drug Combination—Loading a Screw-in Electrical Lead—Drug Combination/Polymer Dip Coating

A polyurethane solution (CHRONOFLEX AL 85 A) is prepared by dissolving 20 g of the polyurethane in 200 ml tetrahydrofuran (THF). 10 ml aliquots of this solution are placed in 20 ml glass scintillation vials. 1 mg, 5 mg, 10 mg, 20 mg, 50 mg, 75 mg, 100 mg, 200 mg, and 500 mg of a drug combination of amoxapine and prednisolone are then added to each of the vials, respectively. The solutions are tumbled for 3 hours at 20 rpm. The tip of an electrical pacing lead that comprises a screw in electrode tip (e.g., CAPSUREFIX NOVUS 5076, Medtronic, Inc.) is screwed into the end of a silastic rod until the screw-in portion is completely incorporated into the silastic rod. The 0.1 mg/ml solution of amoxapine and prednisolone is placed in a thin glass tube that is sealed at one end. The electrical lead is dipped into the solution and is then gradually withdrawn from the solution. The coated electrode is clamped such that the coated portion is suspended in the air. The coating is then air dried for 1 hour. The electrode is unscrewed from the silastic rod. The electrode is further dried under vacuum for 24 hours. The process is repeated for all the prepared solutions of amoxapine and prednisolone using a fresh electrode each time.

In addition to the drug combination of amoxapine and prednisolone, further exemplary drug combinations that may be used to load the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 37

### Screening Assay for Assessing the Effect of Various Anti-Scarring Agents on Nitric Oxide Production by Macrophages

The murine macrophage cell line RAW 264.7 is trypsinized to remove cells from flasks and plated in individual wells of a 6-well plate. Approximately 2×10⁶ cells are plated in 2 ml of media containing 5% heat-inactivated fetal bovine serum (FBS). RAW 264.7 cells are incubated at 37° C. for 1.5 hours to allow adherence to plastic. The agent is prepared in DMSO at a concentration of 10⁻² M and serially diluted 10-fold to give a range of stock concentrations (10⁻⁸ M to 10⁻² M). Media is then removed and cells are incubated in 1 ng/ml of recombinant murine IFNγ and 5 ng/ml of LPS with or without the agent in fresh media containing 5% FBS. The agent is added to cells by directly adding agent DMSO stock solutions, prepared earlier, at a 1/1000 dilution, to each well. Plates containing IFNγ, LPS plus or minus the agent are incubated at 37° C. for 24 hours (Chem. Ber. (1879) 12: 426; J. AOAC (1977) 60-594; Ann. Rev. Biochem. (1994) 63: 175).

At the end of the 24-hour period, supernatants are collected from the cells and assayed for the production of nitrites. Each sample is tested in triplicate by aliquoting 50 μL of supernatant in a 96-well plate and adding 50 μL of Greiss Reagent A (0.5 g sulfanilamide, 1.5 ml H₃PO₄, 48.5 ml ddH₂O) and 50 μL of Greiss Reagent B (0.05 g N-(1-naphthyl)-ethylenediamine, 1.5 ml H₃PO₄, 48.5 ml ddH₂O). Optical density is read immediately on microplate spectrophotometer at 562 nm absorbance. Absorbance over triplicate wells is averaged after subtracting background and concentration values obtained from the nitrite standard curve (1 μM to 2 mM). Inhibitory concentration of 50% (IC₅₀) is determined by comparing average nitrite concentration to the positive control (cell stimulated with IFNγ and LPS). An average of n=4 replicate experiments is used to determine IC₅₀ values for the agent. Exemplary drug combinations, or individual components thereof, that may be tested for IC₅₀ values in this assay include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 38

### Screening Assay for Assessing the Effect of Various Anti-Scarring Agents on TNF-Alpha Production by Macrophages

The human macrophage cell line, THP-1 was plated in a 12 well plate such that each well contains 1×10⁶ cells in 2 ml of media containing 10% FCS. Opsonized zymosan was prepared by resuspending 20 mg of zymosan A in 2 ml of ddH₂O and homogenizing until a uniform suspension was obtained. Homogenized zymosan was pelleted at 250 g and resuspended in 4 ml of human serum for a final concentration of 5 mg/ml and incubated in a 37° C. water bath for 20 minutes to enable opsonization. An agent is prepared in DMSO at a concentration of 10⁻² M and serially diluted 10-fold to give a range of stock concentrations (10⁻⁸ M to 10⁻² M) (J. Immunol. (2000) 165: 411-418; J. Immunol. (2000) 164: 4804-4811; J. Immunol. Meth. (2000) 235 (1-2): 33-40).

THP-1 cells are stimulated to produce TNFα by the addition of 1 mg/ml opsonized zymosan. The agent is added to THP-1 cells by directly adding DMSO stock solutions, prepared earlier, at a 1/1000 dilution, to each well. Each drug concentration was tested in triplicate wells. Plates were incubated at 37° C. for 24 hours.

After 24 hour stimulation, supernatants are collected to quantify TNFα production. TNFα concentrations in the supernatants are determined by ELISA using recombinant human TNFα to obtain a standard curve. A 96-well MaxiSorb plate is coated with 100 μL of anti-human TNFα Capture Antibody diluted in Coating Buffer (0.1M sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% TWEEN-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/8 and 1/16; (b) recombinant human TNFα is prepared at 500 pg/ml and serially diluted to yield as standard curve of 7.8 pg/ml to 500 pg/ml. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human TNFα detection antibody+avidin-HRP) for 1 hour at room temperature. Following this incubation, the plates are washed 7 times and 100 μL of Substrate Solution (tetramethylbenzidine, H₂O₂) is added to plates and incubated for 30 minutes at room temperature. Stop Solution (2 NH₂SO₄) is then added to the wells and a yellow color reaction is read at 450 nm with λ correction at 570 nm. Mean absorbance is determined from triplicate data readings and the mean background is subtracted. TNFα concentration values are obtained from the standard curve. Inhibitory concentration of 50% (IC₅₀) is determined by comparing average TNFα concentration to the positive control (THP-1 cells stimulated with opsonized zymosan). An average of n=4 replicate experiments are used to determine IC₅₀ values. Exemplary drug combinations, or individual components thereof, that may be tested for IC₅₀ values in this assay include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 39

### Surgical Adhesions Model to Assess Fibrosis-Inhibiting Agents in Rats

The rat caecal sidewall model is used to as to assess the anti-fibrotic capacity of formulations in vivo. Sprague Dawley rats are anesthetized with halothane. Using aseptic precautions, the abdomen is opened via a midline incision. The caecum is exposed and lifted out of the abdominal cavity. Dorsal and ventral aspects of the caecum are successively scraped a total of 45 times over the terminal 1.5 cm using a #10 scalpel blade. Blade angle and pressure are controlled to produce punctate bleeding while avoiding severe tissue damage. The left side of the abdomen is retracted and everted to expose a section of the peritoneal wall that lies proximal to the caecum. The superficial layer of muscle (transverses abdominis) is excised over an area of 1×2 cm², leaving behind torn fibers from the second layer of muscle (internal oblique muscle). Abraded surfaces are tamponaded until bleeding stops. The abraded caecum is then positioned over the sidewall wound and attached by two sutures. The formulation is applied over both sides of the abraded caecum and over the abraded peritoneal sidewall. A further two sutures are placed to attach the caecum to the injured sidewall by a total of 4 sutures and the abdominal incision is closed in two layers. After 7 days, animals are evaluated post mortem with the extent and severity of adhesions being scored both quantitatively and qualitatively. Exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 40

### Surgical Adhesions Model to Assess Fibrosis-Inhibiting Agents in Rabbits

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively. Exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 41

### Screening Assay for Assessing the Effect of Various Anti-Scarring Agents on Cell Proliferation

Fibroblasts at 70-90% confluency are trypsinized, replated at 600 cells/well in media in 96-well plates and allowed to attach overnight. The agent is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and the agent are incubated at 37° C. for 72 hours (In vitro toxicol. (1990) 3: 219; Biotech. Histochem. (1993) 68: 29; Anal. Biochem. (1993) 213: 426).

To terminate the assay, the media is removed by gentle aspiration. A 1/400 dilution of CYQUANT 400×GR dye indicator (Molecular Probes; Eugene, Oreg.) is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Inhibitory concentration of 50% (IC₅₀) is determined by taking the average of triplicate wells and comparing average relative fluorescence units to the DMSO control. An average of n=4 replicate experiments is used to determine IC₅₀ values. Exemplary drug combinations, or individual components thereof, that may be tested for IC₅₀ values in this assay include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 42

### Evaluation of Drug Combination—Containing Mesh on Intimal Hyperplasia Development in a Rat Balloon Injury Carotid Artery Model as an Example to Evaluate Fibrosis-Inhibiting Agents

A rat balloon injury carotid artery model was used to demonstrate the efficacy of a drug combination (amoxapine and prednisolone) containing mesh system on the development of intimal hyperplasia fourteen days following placement.

Control Group

Wistar rats weighing 400-500 g were anesthetized with 1.5% halothane in oxygen and the left external carotid artery was exposed. An A 2 French FOGARTY balloon embolectomy catheter (Baxter, Irvine, Calif.) was advanced through an arteriotomy in the external carotid artery down the left common carotid artery to the aorta. The balloon was inflated with enough saline to generate slight resistance (approximately 0.02 ml) and it was withdrawn with a twisting motion to the carotid bifurcation. The balloon was then deflated and the procedure repeated twice more. This technique produced distension of the arterial wall and denudation of the endothelium. The external carotid artery was ligated after removal of the catheter. The right common carotid artery was not injured and was used as a control.

Local Perivascular Drug Combination Treatment

Immediately after injury of the left common carotid artery, a 1 cm long distal segment of the artery is exposed and treated with a 1×1 cm drug combination-containing mesh. The wound is then closed the animals were kept for 14 days.

Histology and Immunohistochemistry

At the time of sacrifice, the animals are euthanized with carbon dioxide and pressure perfused at 100 mmHg with 10% phosphate buffered formaldehyde for 15 minutes. Both carotid arteries are harvested and left overnight in fixative. The fixed arteries are processed and embedded in paraffin wax. Serial cross-sections are cut at 3 μm thickness every 2 mm within and outside the implant region of the injured left carotid artery and at corresponding levels in the control right carotid artery. Cross-sections are stained with Mayer's hematoxylin-and-eosin for cell count and with Movat's pentachrome stains for morphometry analysis and for extracellular matrix composition assessment.

Other exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 43

### Effect of Paclitaxel and Other Anti-Microtubule Agents on Matrix Metalloproteinase Production

A. Materials and Methods

1. IL-1 Stimulated AP-1 Transcriptional Activity is Inhibited by Paclitaxel

Chondrocytes were transfected with constructs containing an AP-1 driven CAT reporter gene, and stimulated with IL-1, IL-1 (50 ng/ml) was added and incubated for 24 hours in the absence and presence of paclitaxel at various concentrations. Paclitaxel treatment decreased CAT activity in a concentration dependent manner (mean±SD). The data noted with an asterisk (*) have significance compared with IL-1-induced CAT activity according to a t-test, P<0.05. The results shown are representative of three independent experiments.

2. Effect of Paclitaxel on IL-1 Induced AP-1 DNA Binding Activity AP-1 DNA

Binding activity was assayed with a radiolabeled human AP-1 sequence probe and gel mobility shift assay. Extracts from chondrocytes untreated or treated with various amounts of paclitaxel (10⁻⁷ to 10⁻⁵ M) followed by IL-1β (20 ng/ml) were incubated with excess probe on ice for 30 minutes, followed by non-denaturing gel electrophoresis. The “com” lane contains excess unlabeled AP-1 oligonucleotide. The results shown are representative of three independent experiments.

3. Effect of Paclitaxel on IL-1 Induced MMP-1 and MMP-3 mRNA Expression

Cells were treated with paclitaxel at various concentrations (10⁻⁷ to 10⁻⁵ M) for 24 hours, then treated with IL-1β (20 ng/ml) for additional 18 hours in the presence of paclitaxel. Total RNA was isolated, and the MMP-1 mRNA levels were determined by Northern blot analysis. The blots were subsequently stripped and reprobed with ³²P-radiolabeled rat GAPDH cDNA, which was used as a housekeeping gene. The results shown are representative of four independent experiments. Quantitation of collagenase-1 and stromelysin-expression mRNA levels was conducted. The MMP-1 and MMP-3 expression levels were normalized with GAPDH.

4. Effect of Other Anti-Microtubules on Collagenase Expression

Primary chondrocyte cultures were freshly isolated from calf cartilage. The cells were plated at 2.5×10⁶ per ml in 100×20 mm culture dishes and incubated in Ham's F12 medium containing 5% FBS overnight at 37° C. The cells were starved in serum-free medium overnight and then treated with anti-microtubule agents at various concentrations for 6 hours. IL-1 (20 ng/ml) was then added to each plate and the plates incubated for an additional 18 hours. Total RNA was isolated by the acidified guanidine isothiocyanate method and subjected to electrophoresis on a denatured gel. Denatured RNA samples (15 μg) were analyzed by gel electrophoresis in a 1% denatured gel, transferred to a nylon membrane and hybridized with the ³²P-labeled collagenase cDNA probe. ³²P-labeled glyceraldehyde phosphate dehydrase (GAPDH) cDNA as an internal standard to ensure roughly equal loading. The exposed films were scanned and quantitatively analyzed with IMAGEQUANT.

B. Results

1. Promoters on the Family of Matrix Metalloproteinases

FIG. 4A shows that all matrix metalloproteinases contained the transcriptional elements AP-1 and PEA-3 with the exception of gelatinase B. It has been well established that expression of matrix metalloproteinases such as collagenases and stromelysins are dependent on the activation of the transcription factors AP-1. Thus inhibitors of AP-1 may inhibit the expression of matrix metalloproteinases.

2. Effect of Paclitaxel on AP-1 Transcriptional Activity

As demonstrated in FIG. 4B, IL-1 stimulated AP-1 transcriptional activity 5-fold. Pretreatment of transiently transfected chondrocytes with paclitaxel reduced IL-1 induced AP-1 reporter gene CAT activity. Thus, IL-1 induced AP-1 activity was reduced in chondrocytes by paclitaxel in a concentration dependent manner (10⁻⁷ to 10⁻⁵ M). These data demonstrated that paclitaxel was a potent inhibitor of AP-1 activity in chondrocytes.

3. Effect of Paclitaxel on AP-1 DNA Binding Activity

To confirm that paclitaxel inhibition of AP-1 activity was not due to nonspecific effects, the effect of paclitaxel on IL-1 induced AP-1 binding to oligonucleotides using chondrocyte nuclear lysates was examined. As shown in FIG. 4C, IL-1 induced binding activity decreased in lysates from chondrocyte which had been pretreated with paclitaxel at concentration 10⁻⁷ to 10⁻⁵ M for 24 hours. Paclitaxel inhibition of AP-1 transcriptional activity closely correlated with the decrease in AP-1 binding to DNA.

4. Effect of Paclitaxel on Collagenase and Stromelysin Expression

Since paclitaxel was a potent inhibitor of AP-1 activity, the effect of paclitaxel or IL-1 induced collagenase and stromelysin expression, two important matrix metalloproteinases involved in inflammatory diseases was examined. Briefly, as shown in FIG. 4D, IL-1 induction increases collagenase and stromelysin mRNA levels in chondrocytes. Pretreatment of chondrocytes with paclitaxel for 24 hours significantly reduced the levels of collagenase and stromelysin mRNA. At 10⁻⁵ M paclitaxel, there was complete inhibition. The results show that paclitaxel completely inhibited the expression of two matrix metalloproteinases at concentrations similar to which it inhibits AP-1 activity.

5. Effect of Other Anti-Microtubules on Collagenase Expression

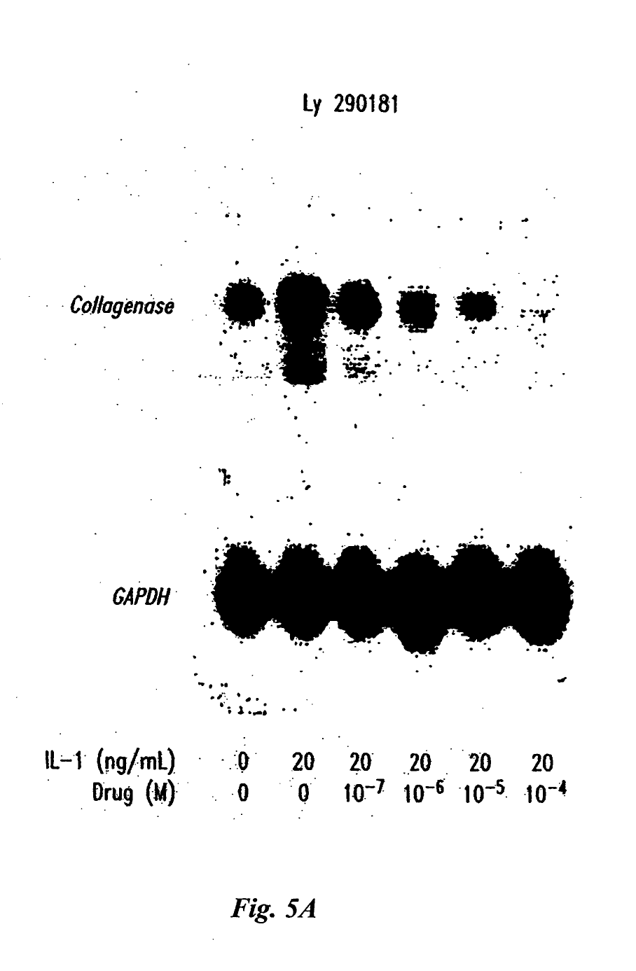

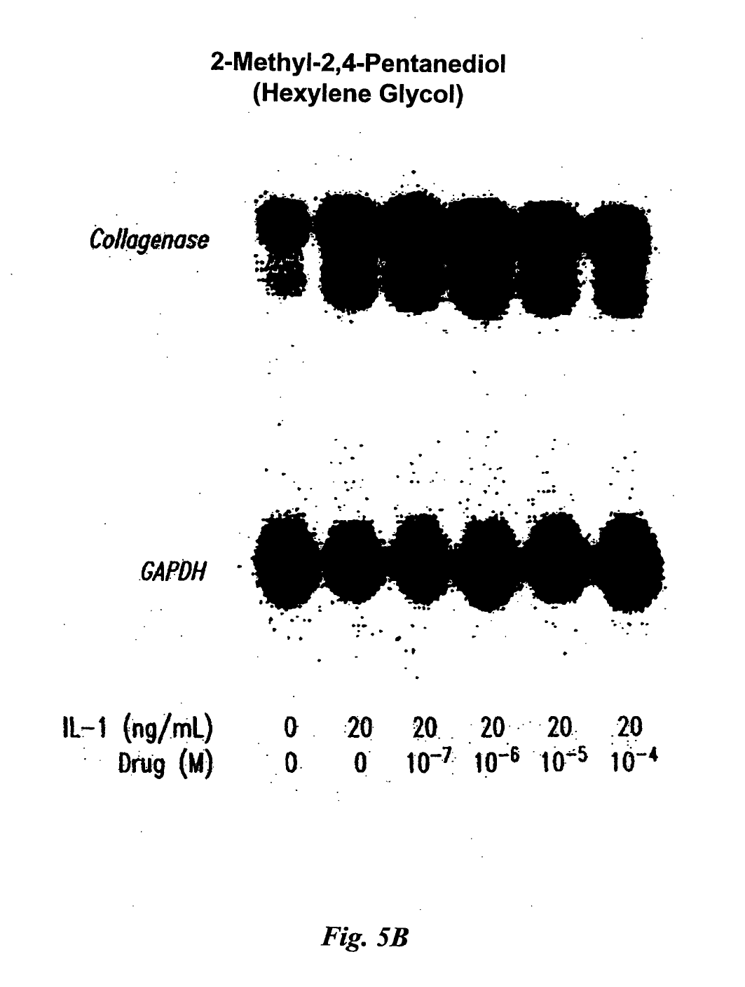

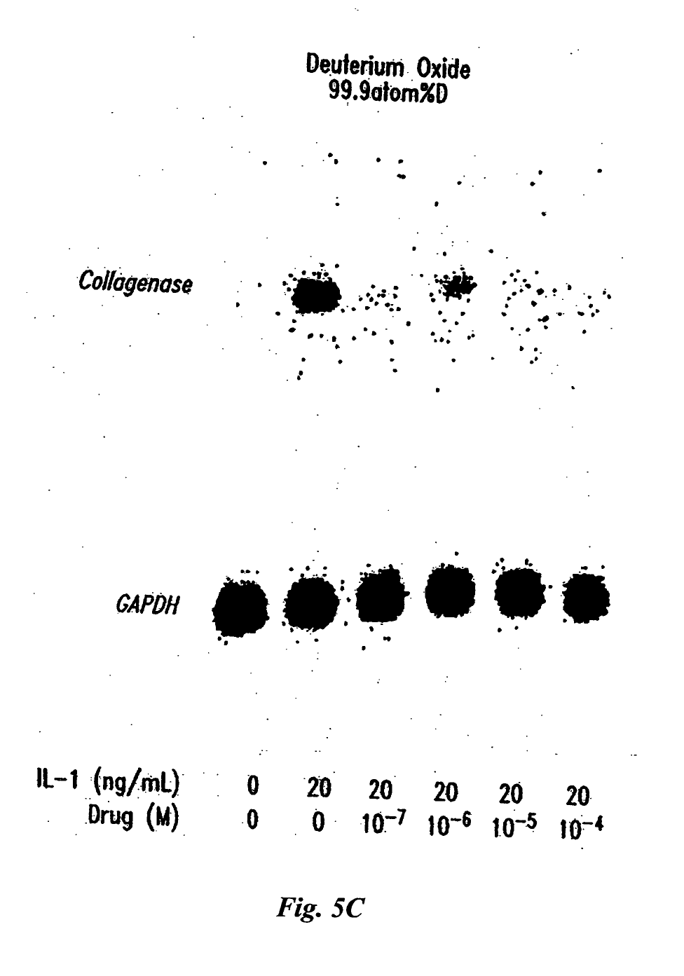

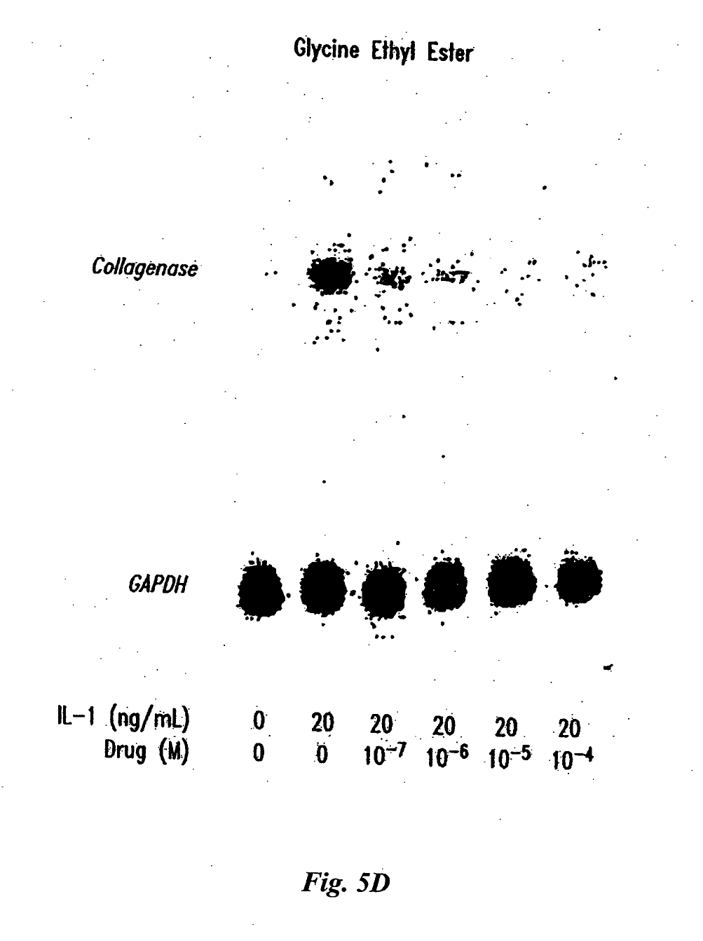

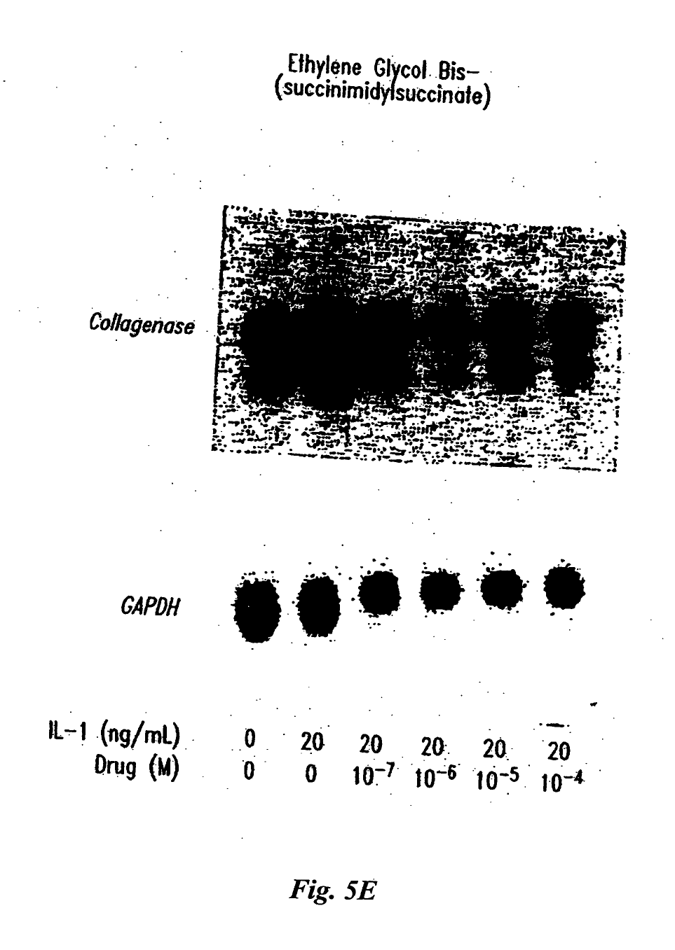

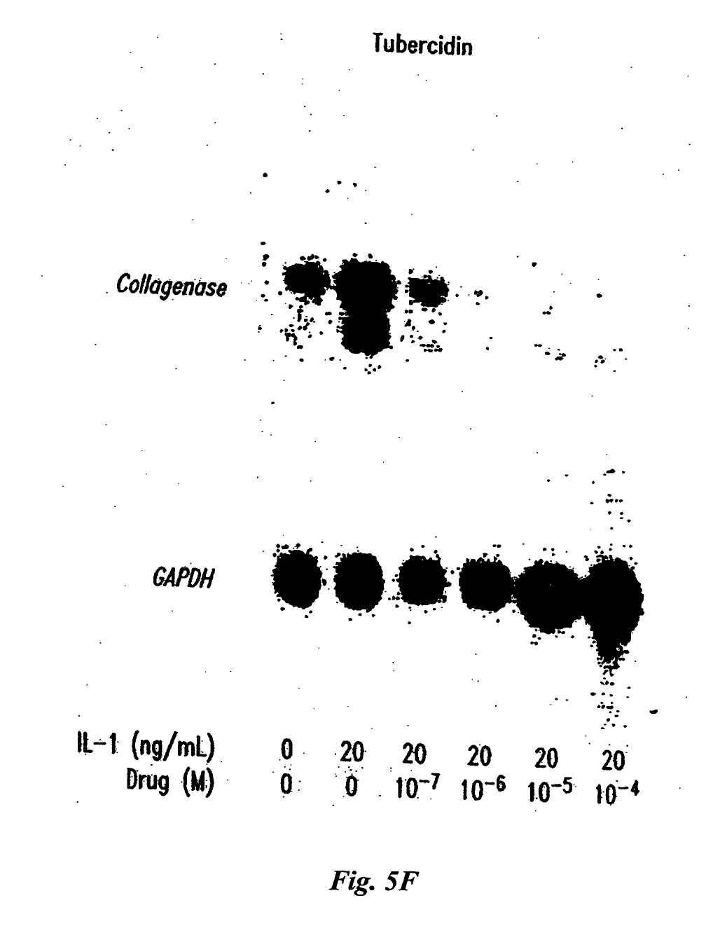

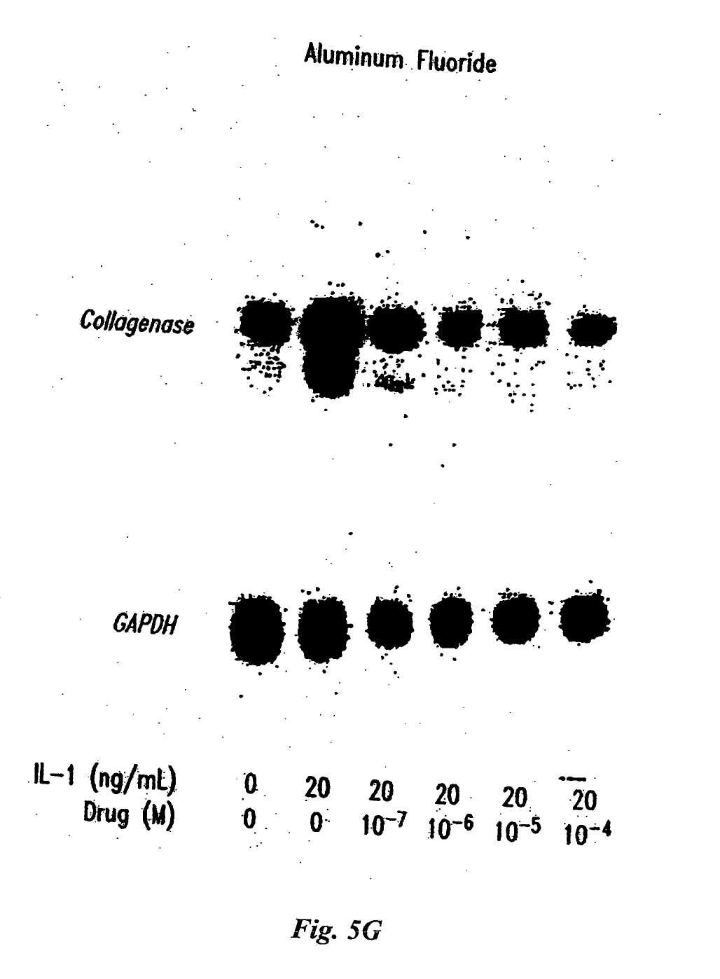

FIGS. 5A-H demonstrate that anti-microtubule agents inhibited collagenase expression. Expression of collagenase was stimulated by the addition of IL-1 which is a proinflammatory cytokine. Pre-incubation of chondrocytes with various anti-microtubule agents, specifically LY290181, hexylene glycol, deuterium oxide, glycine ethyl ester, ethylene glycol bis-(succinimidylsuccinate), tubercidin, AIF₃, and epothilone, all prevented IL-1-induced collagenase expression at concentrations as low as 1×10⁻⁷ M.

C. Discussion

Paclitaxel was capable of inhibiting collagenase and stromelysin expression in vitro at concentrations of 10⁻⁶ M. Since this inhibition may be explained by the inhibition of AP-1 activity, a required step in the induction of all matrix metalloproteinases with the exception of gelatinase B, it is expected that paclitaxel may inhibit other matrix metalloproteinases which are AP-1 dependent. The levels of these matrix metalloproteinases are elevated in all inflammatory diseases and play a principle role in matrix degradation, cellular migration and proliferation, and angiogenesis. Thus, paclitaxel inhibition of expression of matrix metalloproteinases such as collagenase and stromelysin can have a beneficial effect in inflammatory diseases.

In addition to paclitaxel's inhibitory effect on collagenase expression, LY290181, hexylene glycol, deuterium oxide, glycine ethyl ester, AIF₃, tubercidin epothilone, and ethylene glycol bis-(succinimidylsuccinate), all prevented IL-1-induced collagenase expression at concentrations as low as 1×10⁻⁷ M. Thus, anti-microtubule agents are capable of inhibiting the AP-1 pathway at varying concentrations.

### Example 44

### Inhibition of Angiogenesis by Paclitaxel

C. Chick Chorioallantoic Membrane (“CAM”) Assays

Fertilized, domestic chick embryos were incubated for 3 days prior to shell-less culturing. In this procedure, the egg contents were emptied by removing the shell located around the air space. The interior shell membrane was then severed and the opposite end of the shell was perforated to allow the contents of the egg to gently slide out from the blunted end. The egg contents were emptied into round-bottom sterilized glass bowls and covered with petri dish covers. These were then placed into an incubator at 90% relative humidity and 3% CO₂ and incubated for 3 days.

Paclitaxel (Sigma, St. Louis, Mich.) was mixed at concentrations of 0.25, 0.5, 1, 5, 10, 30 μg per 10 ul aliquot of 0.5% aqueous methylcellulose. Since paclitaxel is insoluble in water, glass beads were used to produce fine particles. Ten microliter aliquots of this solution were dried on parafilm for 1 hour forming disks 2 mm in diameter. The dried disks containing paclitaxel were then carefully placed at the growing edge of each CAM at day 6 of incubation. Controls were obtained by placing paclitaxel-free methylcellulose disks on the CAMs over the same time course. After a 2 day exposure (day 8 of incubation) the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

The membranes of the 8 day-old shell-less embryo were flooded with 2% glutaraldehyde in 0.1M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were postfixed in 1% osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding, Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5 minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501B scanning electron microscope.

Results of the assay were as follows. At day 6 of incubation, the embryo was centrally positioned to a radially expanding network of blood vessels; the CAM developed adjacent to the embryo. These growing vessels lie close to the surface and are readily visible making this system an idealized model for the study of angiogenesis. Living, unstained capillary networks of the CAM may be imaged noninvasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg, each CAM was examined under living conditions with a stereomicroscope equipped with a video/computer interface in order to evaluate the effects on angiogenesis. This imaging setup was used at a magnification of 160× which permitted the direct visualization of blood cells within the capillaries; thereby blood flow in areas of interest may be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (Table 11). This scale represents the degree of overall inhibition with maximal inhibition represented as a 3 on the avascular gradient scale. Paclitaxel was very consistent and induced a maximal avascular zone (6 mm in diameter or a 3 on the avascular gradient scale) within 48 hours depending on its concentration.

TABLE 11
Avascular Gradient

| | |
|---|---|
| 0 | normal vascularity |
| 1 | lacking some microvascular movement |
| 2* | small avascular zone approximately 2 mm in diameter |
| 3* | avascularity extending beyond the disk (6 mm in diameter) |

*indicates a positive antiangiogenesis response

The dose-dependent, experimental data of the effects of paclitaxel at different concentrations are shown in Table 12.

TABLE 12

| Agent | Delivery Vehicle | Concentration | | Inhibition/n |
|---|---|---|---|---|
| paclitaxel | methylcellulose (10 ul) | 0.25 | ug | 2/11 |
| | methylcellulose (10 ul) | 0.5 | ug | 6/11 |
| | methylcellulose (10 ul) | 1 | ug | 6/15 |
| | methylcellulose (10 ul) | 5 | ug | 20/27 |
| | methylcellulose (10 ul) | 10 | ug | 16/21 |
| | methylcellulose (10 ul) | 30 | ug | 31/31 |

Typical paclitaxel-treated CAMs are also shown with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after paclitaxel application to the CAM, angiogenesis was inhibited. The blood vessel inhibition formed an avascular zone which was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

Exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 45

### Screening Assay for Assessing the Effect of Paclitaxel on Smooth Muscle Cell Migration

Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media and diluted to a concentration of 2-2.5×10⁵ cells/ml in migration media. Migration media consists of phenol red free Dulbecco's Modified Eagle Medium (DMEM) containing 0.35% human serum albumin. A 100 μL volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB) was added at a concentration of 10 ng/ml in a total volume of 150 μL. Paclitaxel was prepared in DMSO at a concentration of 10⁻² M and serially diluted 10-fold to give a range of stock concentrations (10⁻⁸ M to 10⁻² M). Paclitaxel was added to cells by directly adding paclitaxel DMSO stock solutions, prepared earlier, at a 1/1000 dilution, to the cells in the top chamber. Plates were incubated for 4 hours to allow cell migration.

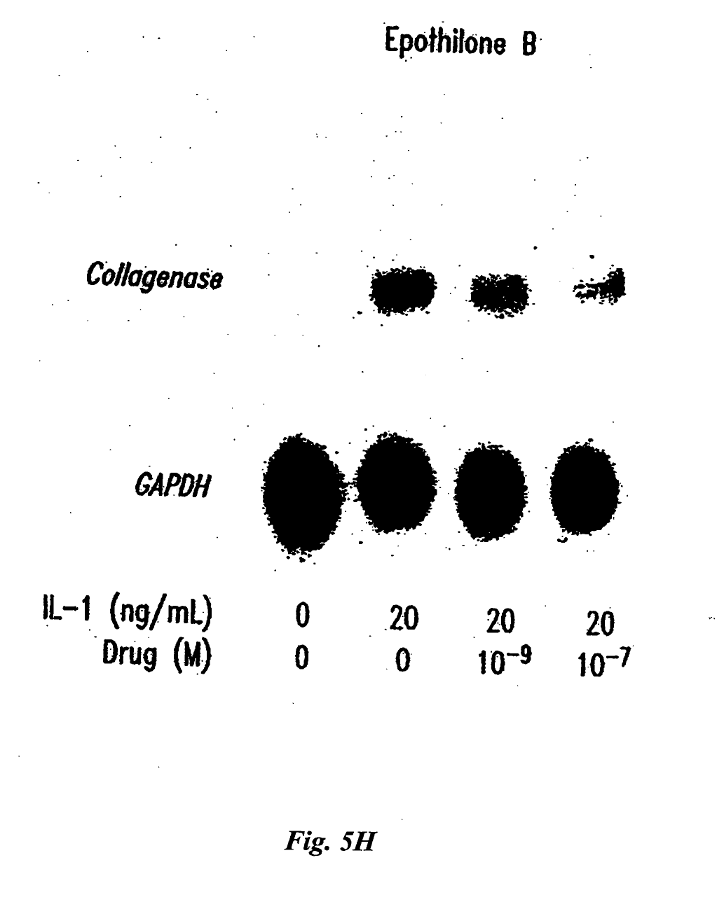

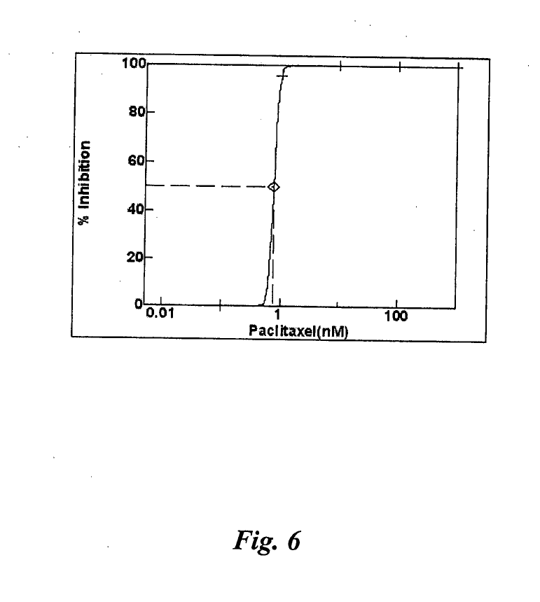

At the end of the 4-hour period, cells in the top chamber were discarded and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and ˜520 nm emission maxima. Relative fluorescence units from triplicate wells were averaged after subtracting background fluorescence (control chamber without chemoattractant) and average number of cells migrating was obtained from a standard curve of smooth muscle cells serially diluted from 25,000 cells/well down to 98 cells/well. Inhibitory concentration of 50% (IC₅₀) was determined by comparing the average number of cells migrating in the presence of paclitaxel to the positive control (smooth muscle cell chemotaxis in response to rhPDGF-BB). See FIG. 6 (IC₅₀=0.76 nM). References: Biotechniques (2000) 29: 81; J. Immunol Methods (2001) 254: 85.

Exemplary compounds that may be tested in this assay include: e.g., ZD-6474, AP-23573, Synthadotin, S-0885, Aplidine, Ixabepilone, IDN-5390, SB-2723005, ABT-518, Combretastatin, Anecortave acetate, SB-715992, Temsirolimus, Adalimumab, erucylphosphocholine, alphastatin, BXT-51072, Etanercept, Humicade, and Gefitinib. Additionally, exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 46

### Screening Assay for Assessing the Effect of Various Drug Combinations on IL-1β Production by Macrophages

The human macrophage cell line, THP-1 is plated in a 12 well plate such that each well contains 1×10⁶ cells in 2 ml of media containing 10% FCS. Opsonized zymosan is prepared by resuspending 20 mg of zymosan A in 2 ml of ddH₂O and homogenizing until a uniform suspension is obtained. Homogenized zymosan is pelleted at 250 g and resuspended in 4 ml of human serum for a final concentration of 5 mg/ml and incubated in a 37° C. water bath for 20 minutes to enable opsonization. A drug combination (amoxapine and prednisolone) is prepared in DMSO at a concentration of 10⁻² M and serially diluted 10-fold to give a range of stock concentrations (10⁻⁸ M to 10⁻² M).

THP-1 cells are stimulated to produce IL-1β by the addition of 1 mg/ml opsonized zymosan. The drug combination is added to THP-1 cells by directly adding DMSO stock solutions, prepared earlier, at a 1/1000 dilution, to each well. Each drug concentration is tested in triplicate wells. Plates are incubated at 37° C. for 24 hours.

After a 24-hour stimulation, supernatants are collected to quantify IL-1β production. IL-1β concentrations in the supernatants are determined by ELISA using recombinant human IL-1β to obtain a standard curve. A 96-well MaxiSorb plate is coated with 100 μL of anti-human IL-1β Capture Antibody diluted in Coating Buffer (0.1M Sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% TWEEN-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/4 and 1/8; (b) recombinant human IL-1β is prepared at 1000 pg/ml and serially diluted to yield a standard curve of 15.6 pg/ml to 1000 pg/ml. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human IL-1β detection antibody+avidin-HRP) for 1 hour at room temperature. Following this incubation, the plates are washed 7 times and 100 μL of Substrate Solution (Tetramethylbenzidine, H₂O₂) is added to plates and incubated for 30 minutes at room temperature. Stop Solution (2 NH₂SO₄) is then added to the wells and a yellow color reaction is read at 450 nm with λ correction at 570 nm. Mean absorbance is determined from triplicate data readings and the mean background is subtracted. IL-1β concentration values are obtained from the standard curve. Inhibitory concentration of 50% (IC₅₀) is determined by comparing average IL-1β concentration to the positive control (THP-1 cells stimulated with opsonized zymosan).

Other exemplary drug combinations, or individual components thereof, that may be tested for IC₅₀ values in this assay include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

References: J. Immunol. (2000) 165: 411-418; J. Immunol. (2000) 164: 4804-4811; J. Immunol Meth. (2000) 235 (1-2): 33-40.

### Example 47

### Screening Assay for Assessing the Effect of Various Compounds on IL-8 Production by Macrophages

The human macrophage cell line, THP-1 is plated in a 12 well plate such that each well contains 1×10⁶ cells in 2 ml of media containing 10% FCS. Opsonized zymosan is prepared by resuspending 20 mg of zymosan A in 2 ml of ddH₂O and homogenizing until a uniform suspension is obtained. Homogenized zymosan is pelleted at 250 g, resuspended in 4 ml of human serum for a final concentration of 5 mg/ml, and incubated in a 37° C. water bath for 20 minutes to enable opsonization. The agent is prepared in DMSO at a concentration of 10⁻² M and serially diluted 10-fold to give a range of stock concentrations (10⁻⁸ M to 10⁻² M).

THP-1 cells are stimulated to produce IL-8 by the addition of 1 mg/ml opsonized zymosan. Geldanamycin is added to THP-1 cells by directly adding DMSO stock solutions, prepared earlier, at a 1/1000 dilution, to each well. Each drug concentration is tested in triplicate wells. Plates are incubated at 37° C. for 24 hours.

After a 24-hour stimulation, supernatants are collected to quantify IL-8 production. IL-8 concentrations in the supernatants are determined by ELISA using recombinant human IL-8 to obtain a standard curve. A 96-well MAXISORB plate is coated with 100 μL of anti-human IL-8 Capture Antibody diluted in Coating Buffer (0.1M sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% TWEEN-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/100 and 1/1000; (b) recombinant human IL-8 is prepared at 200 pg/ml and serially diluted to yield as standard curve of 3.1 pg/ml to 200 pg/ml. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human IL-8 detection antibody+avidin-HRP) for 1 hour at room temperature. Following this incubation, the plates are washed 7 times and 100 μL of Substrate Solution (Tetramethylbenzidine, H₂O₂) is added to plates and incubated for 30 minutes at room temperature. Stop Solution (2 NH₂SO₄) is then added to the wells and a yellow color reaction is read at 450 nm with λ correction at 570 nm. Mean absorbance is determined from triplicate data readings and the mean background is subtracted. IL-8 concentration values are obtained from the standard curve. Inhibitory concentration of 50% (IC₅₀) is determined by comparing average IL-8 concentration to the positive control (THP-1 cells stimulated with opsonized zymosan).

Exemplary drug combinations, or individual components thereof, that may be tested for IC₅₀ values in this assay include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

References: J. Immunol. (2000) 165: 411-418; J. Immunol. (2000) 164: 4804-4811; J. Immunol Meth. (2000) 235 (1-2): 33-40.

### Example 48

### Screening Assay for Assessing the Effect of Various Drug Combinations on MCP-1 Production by Macrophages

The human macrophage cell line, THP-1 is plated in a 12 well plate such that each well contains 1×10⁶ cells in 2 ml of media containing 10% FCS. Opsonized zymosan is prepared by resuspending 20 mg of zymosan A in 2 ml of ddH₂O and homogenizing until a uniform suspension is obtained. Homogenized zymosan is pelleted at 250 g and resuspended in 4 ml of human serum for a final concentration of 5 mg/ml and incubated in a 37° C. water bath for 20 minutes to enable opsonization. The drug combination (amoxapine and prednisolone) is prepared in DMSO at a concentration of 10⁻² M and serially diluted 10-fold to give a range of stock concentrations (10⁻⁸ M to 10⁻² M).

THP-1 cells are stimulated to produce MCP-1 by the addition of 1 mg/ml opsonized zymosan. The drug combination is added to THP-1 cells by directly adding DMSO stock solutions, prepared earlier, at a 1/1000 dilution, to each well. Each drug concentration is tested in triplicate wells. Plates are incubated at 37° C. for 24 hours.

After 24-hour stimulation, supernatants are collected to quantify MCP-1 production. MCP-1 concentrations in the supernatants are determined by ELISA using recombinant human MCP-1 to obtain a standard curve. A 96-well MaxiSorb plate is coated with 100 μL of anti-human MCP-1 Capture Antibody diluted in Coating Buffer (0.1M sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% TWEEN-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/100 and 1/1000; (b) recombinant human MCP-1 is prepared at 500 pg/ml and serially diluted to yield as standard curve of 7.8 pg/ml to 500 pg/ml. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human MCP-1 detection antibody+avidin-HRP) for 1 hour at room temperature. Following this incubation, the plates are washed 7 times and 100 μL of Substrate Solution (tetramethylbenzidine, H₂O₂) is added to plates and incubated for 30 minutes at room temperature. Stop Solution (2 NH₂SO₄) is then added to the wells and a yellow color reaction was read at 450 nm with k correction at 570 nm. Mean absorbance is determined from triplicate data readings and the mean background is subtracted. MCP-1 concentration values were obtained from the standard curve. Inhibitory concentration of 50% (IC₅₀) is determined by comparing average MCP-1 concentration to the positive control (THP-1 cells stimulated with opsonized zymosan).

Other exemplary drug combinations, or individual components thereof, that may be tested for IC₅₀ values in this assay include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

References: J. Immunol. (2000) 165: 411-418; J. Immunol. (2000) 164: 4804-4811; J. Immunol Meth. (2000) 235 (1-2): 33-40.

### Example 49

### Screening Assay for Assessing the Effect of a Drug Combination on Cell Proliferation

Smooth muscle cells at 70-90% confluency are trypsinized, replated at 600 cells/well in media in 96-well plates and allowed to attachment overnight. The drug combination (amoxapine and prednisolone) is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing cells and paclitaxel are incubated at 37° C. for 72 hours.

To terminate the assay, the media is removed by gentle aspiration. A 1/400 dilution of CYQUANT 400×GR dye indicator (Molecular Probes; Eugene, Oreg.) is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Inhibitory concentration of 50% (IC₅₀) is determined by taking the average of triplicate wells and comparing average relative fluorescence units to the DMSO control. An average of n=3 replicate experiments is used to determine IC₅₀ values.

Other exemplary drug combinations, or individual components thereof, that may be tested for IC₅₀ values in this assay include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

This assay also may be used assess the effect of compounds on proliferation of fibroblasts and murine macrophage cell line RAW 264.7.

Reference: In vitro toxicol. (1990) 3: 219; Biotech. Histochem. (1993) 68: 29; Anal. Biochem. (1993) 213: 426.

### Example 50

### Evaluation of Drug Compound-Containing Mesh on Intimal Hyperplasia Development in a Rat Balloon Injury Carotid Artery Model as an Example to Evaluate Fibrosis-Inhibiting Agents

A rat balloon injury carotid artery model is used to demonstrate the efficacy of a drug combination-containing mesh system on the development of intimal hyperplasia fourteen days following placement.

Control Group

Wistar rats weighing 400-500 g are anesthetized with 1.5% halothane in oxygen and the left external carotid artery was exposed. An A 2 French FOGARTY balloon embolectomy catheter (Baxter, Irvine, Calif.) is advanced through an arteriotomy in the external carotid artery down the left common carotid artery to the aorta. The balloon is inflated with enough saline to generate slight resistance (approximately 0.02 ml) and it is withdrawn with a twisting motion to the carotid bifurcation. The balloon is then deflated and the procedure repeated twice more. This technique produces distension of the arterial wall and denudation of the endothelium. The external carotid artery is ligated after removal of the catheter. The right common carotid artery is not injured and is used as a control.

Local Perivascular Drug Combination Treatment

Immediately after injury of the left common carotid artery, a 1 cm long distal segment of the artery is exposed and treated with a 1×1 cm drug combination-containing mesh. The wound is then closed the animals are kept for 14 days.

Histology and Immunohistochemistry

At the time of sacrifice, the animals are euthanized with carbon dioxide and pressure perfused at 100 mmHg with 10% phosphate buffered formaldehyde for 15 minutes. Both carotid arteries are harvested and left overnight in fixative. The fixed arteries are processed and embedded in paraffin wax. Serial cross-sections are cut at 3 μm thickness every 2 mm within and outside the implant region of the injured left carotid artery and at corresponding levels in the control right carotid artery. Cross-sections are stained with Mayer's hematoxylin-and-eosin for cell count and with Movat's pentachrome stains for morphometry analysis and for extracellular matrix composition assessment.

Other exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 51

### In Vivo Evaluation of Silk-Coated Perivascular Polyurethane Films to Assess the Ability of an Agent to Induce Scarring

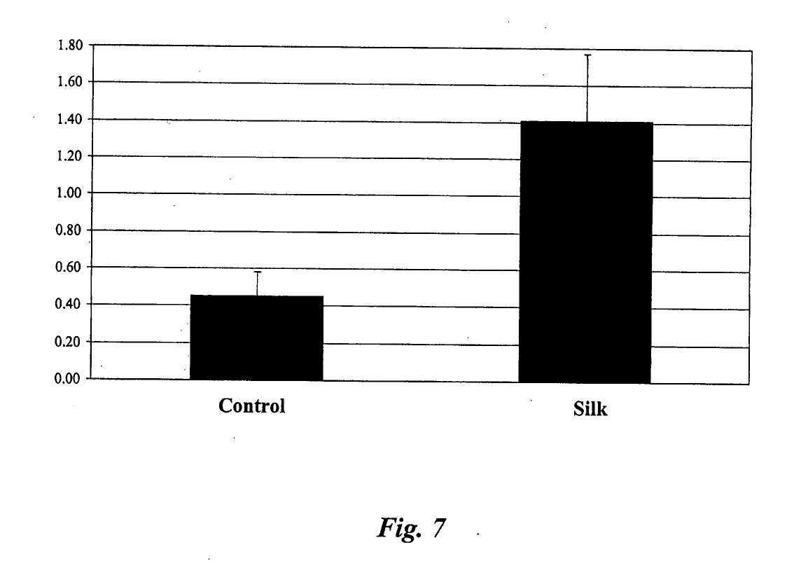

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. A polyurethane (PU) film covered with silk strands or a control uncoated PU film is wrapped around a distal segment of the common carotid artery. The wound is closed and the animal is recovered. After 28 days, the rats are sacrificed with carbon dioxide and pressure-perfused at 100 mmHg with 10% buffered formaldehyde. Both carotid arteries are harvested and processed for histology. Serial cross-sections can be cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Area of the granulation tissue is significantly higher in the silk-coated group than in the control uncoated group. See FIG. 7. Other compounds may also be tested in this manner to assess their ability to induce scarring.

### Example 52

### In Vivo Evaluation of Perivascular Polyurethane Films Coated with Different Silk Suture Material to Assess Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. A polyurethane film covered with silk sutures from one of three different manufacturers (3-0 Silk-Black Braided (Davis & Geck), 3-0 SOFSILK (U.S. Surgical/Davis & Geck), and 3-0 Silk-Black Braided (LIGAPAK) (Ethicon, Inc.) is wrapped around a distal segment of the common carotid artery. (The polyurethane film can also be coated with other agents to induce fibrosis.) The wound is closed and the animal is allowed to recover.

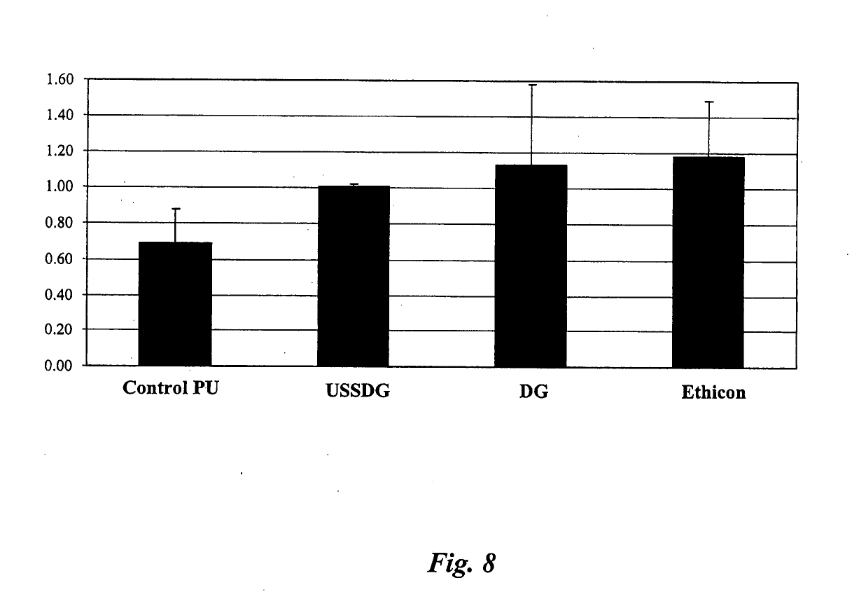

After 28 days, the rats are sacrificed with carbon dioxide and pressure-perfused at 100 mmHg with 10% buffered formaldehyde. Both carotid arteries are harvested and processed for histology. Serial cross-sections are cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Thickness of the granulation tissue is the same in the three groups showing that tissue proliferation around silk suture is independent of manufacturing processes. See FIG. 8.

### Example 53

### In Vivo Evaluation of Perivascular Silk Powder to Assess the Capacity of an Agent to Induce Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Silk powder is sprinkled on the exposed artery that is then wrapped with a polyurethane (PU) film. Natural silk powder or purified silk powder (without contaminant proteins) is used in different groups of animals. Carotids wrapped with PU films only are used as a control group. The wound is closed and the animal is allowed to recover. After 28 days, the rats are sacrificed with carbon dioxide and pressure-perfused at 100 mmHg with 10% buffered formaldehyde. Both carotid arteries are harvested and processed for histology. Serial cross-sections can be cut every 2 mm in the treated left carotid artery and at corresponding levels in the untreated right carotid artery. Sections are stained with H&E and Movat's stains to evaluate tissue growth around the carotid artery. Area of tunica intima, tunica media and perivascular granulation tissue is quantified by computer-assisted morphometric analysis.

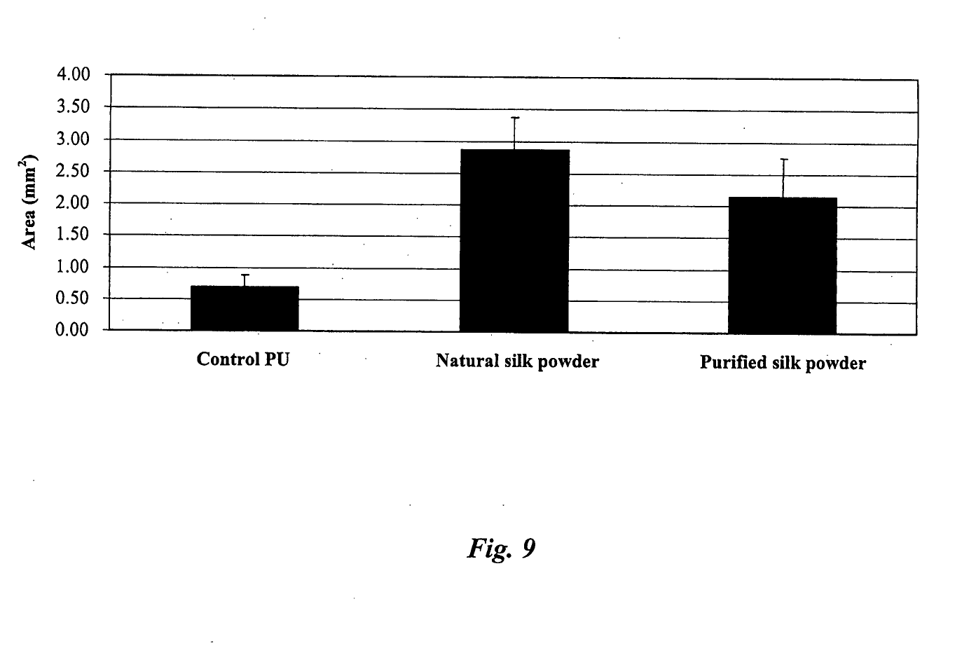

The natural silk caused a severe cellular inflammation consisting mainly of a neutrophil and lymphocyte infiltrate in a fibrin network without any extracellular matrix or blood vessels. In addition, the treated arteries were seriously damaged with hypocellular media, fragmented elastic laminae and thick intimal hyperplasia. Intimal hyperplasia contained many inflammatory cells and was occlusive in 2/6 cases. This severe immune response was likely triggered by antigenic proteins coating the silk protein in this formulation. On the other end, the regenerated silk powder triggered only a mild foreign body response surrounding the treated artery. This tissue response was characterized by inflammatory cells in extracellular matrix, giant cells and blood vessels. The treated artery was intact. These results show that removing the coating proteins from natural silk prevents the immune response and promotes benign tissue growth. Degradation of the regenerated silk powder was underway in some histology sections indicating that the tissue response can likely mature and heal over time. See FIG. 9.

### Example 54

### In Vivo Evaluation of Perivascular Talcum Powder to Assess the Capacity of an Agent to Induce Scarring

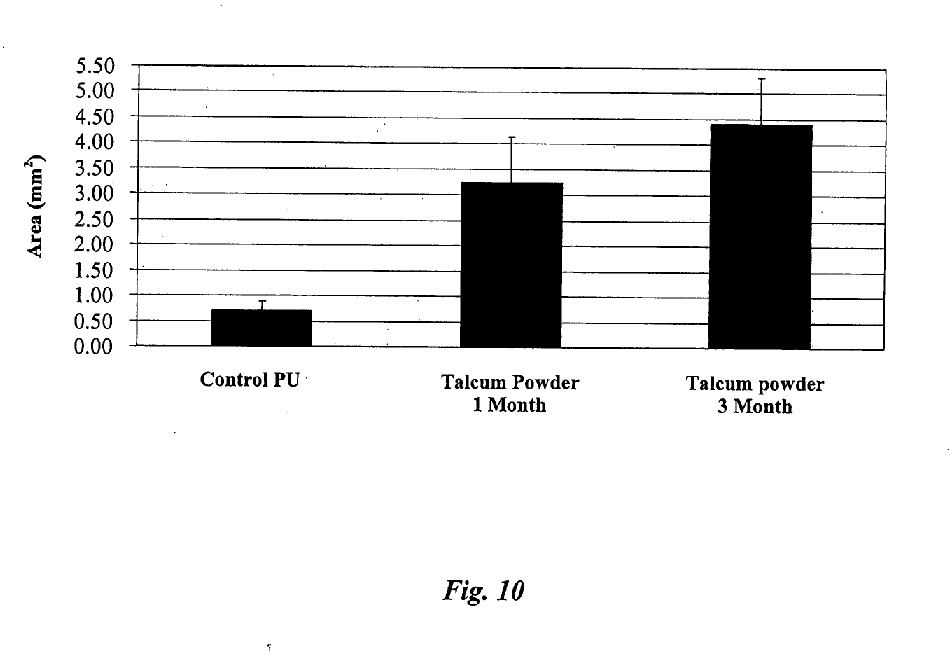

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Talcum powder is sprinkled on the exposed artery that is then wrapped with a polyurethane (PU) film. Carotids wrapped with PU films only are used as a control group. The wound is closed and the animal is recovered. After 1 or 3 months, the rats are sacrificed with carbon dioxide and pressure-perfused at 100 mmHg with 10% buffered formaldehyde. Both carotid arteries are harvested and processed for histology. Serial cross-sections are cut every 2 mm in the treated left carotid artery and at corresponding levels in the untreated right carotid artery. Sections are stained with H&E and Movat's stains to evaluate tissue growth around the carotid artery. Thickness of tunica intima, tunica media and perivascular granulation tissue is quantified by computer-assisted morphometric analysis. Histopathology results and morphometric analysis showed the same local response to talcum powder at 1 month and 3 months. A large tissue reaction trapped the talcum powder at the site of application around the blood vessel. This tissue was characterized by a large number of macrophages within a dense extracellular matrix with few neutrophiles, lymphocytes and blood vessels. The treated blood vessel appeared intact and unaffected by the treatment. Overall, this result showed that talcum powder induced a mild long-lasting fibrotic reaction that was subclinical in nature and did not harm any adjacent tissue. See FIG. 10.

### Example 55

### MIC Determination by Microtitre Broth Dilution Method

A. MIC Assay of Various Gram Negative and Positive Bacteria

MIC assays were conducted essentially as described by Amsterdam, D. 1996, “Susceptibility testing of antimicrobials in liquid media”, p. 52-11, in Loman, V., ed. Antibiotics in laboratory medicine, 4th ed. Williams and Wilkins, Baltimore, Md. Briefly, a variety of compounds were tested for antibacterial activity against isolates of P. aeruginosa, K. pneumoniae, E. coli, S. epidermidis and S. aureus in the MIC (minimum inhibitory concentration assay under aerobic conditions using 96 well polystyrene microtitre plates (Falcon 1177), and Mueller Hinton broth at 37° C. incubated for 24 h. (MHB was used for most testing except C721 (S. pyogenes), which used Todd Hewitt broth, and Haemophilus influenzae, which used Haemophilus test medium (HTM)) Tests were conducted in triplicate. The results are provided below in Table 13.

TABLE 13
MINIMUM INHIBITORY CONCENTRATIONS OF THERAPEUTIC AGENTS
AGAINST VARIOUS GRAM NEGATIVE AND POSITIVE BACTERIA

| | Bactrial Strain | | | | | |
|---|---|---|---|---|---|---|
| | P. aeruginosa | K. pneumoniae | E. coli | S. aureus | | |
| | PAE/K799 | ATCC13883 | UB1005 | ATCC25923 | S. epidermidis | S. pyogenes |
| | H187 | C238 | C498 | C622 | C621 | C721 |
| | Wt | wt | wt | wt | wt | wt |
| Drug | Gram− | Gram− | Gram− | Gram+ | Gram+ | Gram+ |
| doxorubicin | 10⁻⁵ | 10⁻⁶ | 10⁻⁴ | 10⁻⁵ | 10⁻⁶ | 10⁻⁷ |
| mitoxantrone | 10⁻⁵ | 10⁻⁶ | 10⁻⁵ | 10⁻⁵ | 10⁻⁵ | 10⁻⁶ |
| 5-fluorouracil | 10⁻⁵ | 10⁻⁶ | 10⁻⁶ | 10⁻⁷ | 10⁻⁷ | 10⁻⁴ |
| methotrexate | N | 10⁻⁶ | N | 10⁻⁵ | N | 10⁻⁶ |
| etoposide | N | 10⁻⁵ | N | 10⁻⁵ | 10⁻⁶ | 10⁻⁵ |
| camptothecin | N | N | N | N | 10⁻⁴ | N |
| hydroxyurea | 10⁻⁴ | N | N | N | N | 10⁻⁴ |
| cisplatin | 10⁻⁴ | N | N | N | N | N |
| tubercidin | N | N | N | N | N | N |
| 2-mercaptopurine | N | N | N | N | N | N |
| 6-mercaptopurine | N | N | N | N | N | N |
| cytarabine | N | N | N | N | N | N |

Activities are in Molar concentrations
Wt = wild type
N = No activity

B. MIC of Antibiotic-Resistant Bacteria

Various concentrations of the following compounds, mitoxantrone, cisplatin, tubercidin, methotrexate, 5-fluorouracil, etoposide, 2-mercaptopurine, doxorubicin, 6-mercaptopurine, camptothecin, hydroxyurea and cytarabine were tested for antibacterial activity against clinical isolates of a methicillin resistant S. aureus and a vancomycin resistant pediococcus clinical isolate in an MIC assay as described above. Compounds which showed inhibition of growth (MIC value of <1.0×10−3) included: mitoxantrone (both strains), methotrexate (vancomycin resistant pediococcus), 5-fluorouracil (both strains), etoposide (both strains), and 2-mercaptopurine (vancomycin resistant pediococcus).

### Example 56

### Preparation of Release Buffer

The release buffer is prepared by adding 8.22 g sodium chloride, 0.32 g sodium phosphate monobasic (monohydrate) and 2.60 g sodium phosphate dibasic (anhydrous) to a beaker. 1 L HPLC grade water is added and the solution is stirred until all the salts are dissolved. If required, the pH of the solution is adjusted to pH 7.4±0.2 using either 0.1N NaOH or 0.1N phosphoric acid.

### Example 57

### Release Study to Determine Release Profile of a Drug Combination from a Coated Device

A sample of a drug combination-loaded electrical lead is placed in a 15 ml culture tube. 15 ml release buffer (Example 57) is added to the culture tube. The tube is sealed with a TEFLON lined screw cap and is placed on a rotating wheel in a 37° C. oven. At various time points, the buffer is withdrawn from the culture tube and is replaced with fresh buffer. The withdrawn buffer is then analyzed for the amount of therapeutic agent contained in this buffer solution using HPLC.

Devices analyzed by this method may be coated with exemplary drug combinations, or individual components thereof, that include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 58

### Perivascular Administration of Drug Combination to Assess Inhibition of Fibrosis

WISTAR rats weighing 250-300 g are anesthetized by the intramuscular injection of Innovar (0.33 ml/kg). Once sedated, they are then placed under Halothane anesthesia. After general anesthesia is established, fur over the neck region is shaved, the skin clamped and swabbed with betadine. A vertical incision is made over the left carotid artery and the external carotid artery exposed. Two ligatures are placed around the external carotid artery and a transverse arteriotomy is made. A number 2 French Fogarty balloon catheter is then introduced into the carotid artery and passed into the left common carotid artery and the balloon is inflated with saline. The catheter is passed up and down the carotid artery three times. The catheter is then removed and the ligature is tied off on the left external carotid artery.

A drug combination (33%) in ethylene vinyl acetate (EVA) is then injected in a circumferential fashion around the common carotid artery in ten rats. EVA alone is injected around the common carotid artery in ten additional rats. (The drug combination may also be coated onto an EVA film which is then placed in a circumferential fashion around the common carotid artery.) Five rats from each group are sacrificed at 14 days and the final five at 28 days. The rats are observed for weight loss or other signs of systemic illness. After 14 or 28 days the animals are anesthetized and the left carotid artery is exposed in the manner of the initial experiment. The carotid artery is isolated, fixed at 10% buffered formaldehyde and examined for histology.

A statistically significant reduction in the degree of initimal hyperplasia, as measured by standard morphometric analysis, indicates a drug induced reduction in fibrotic response.

Exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 59

### Spinal Surgical Adhesions Model to Assess Fibrosis-Inhibiting Drug Combinations in Rabbits

Extensive scar formation and adhesions often occur after lumbar spine surgery involving the vertebrae. The dense and thick fibrous tissue adherent to the spine and adjacent muscles must be removed by surgery. Unfortunately, fibrous adhesions usually reform after the secondary surgery. Adhesions are formed by proliferation and migration of fibroblasts from the surrounding tissue at the site of surgery. These cells are responsible for the healing response after tissue injury. Once they have migrated to the wound they lay down proteins such as collagen to repair the injured tissue. Overproliferation and secretion by these cells induce local obstruction, compression and contraction of the surrounding tissues with accompanying side effects.

The rabbit laminectomy spinal adhesion model described herein is used to investigate spinal adhesion prevention by local slow release of antifibrotic drug combinations.

Five to six animals are included in each experimental group to allow for meaningful statistical analysis. Formulations with various concentrations of antifibrotic drug combinations are tested against control animals to assess inhibition of adhesion formation.

Rabbits are anesthetized with an IM injection of ketamine/zylazine. An endotracheal tube is inserted for maintenance of anesthesia with halothane. The animal is placed prone on the operating table on top of a heating pad and the skin over the lower half of the back is shaved and prepared for sterile surgery. A longitudinal midline skin incision is made from L-1 to L-5 and down the lumbosacral fascia. The fascia is incised to expose the tips of the spinous processes. The paraspinous muscles are dissected and retracted from the spinous process and lamina of L-4. A laminectomy is performed at L-4 by removal of the spinal process with careful bilateral excision of the laminae, thus creating a small 5×10 mm laminectomy defect. Hemostasis is obtained with Gelfoam. The test formulations are applied to the injury site and the wound is closed in layers with Vicryl sutures. The animals are placed in an incubator until recovery from anesthesia and then returned to their cage.

Two weeks after surgery, the animals are anesthetized using procedures similar to those described above. The animals are euthanized with Euthanyl. After a skin incision, the laminectomy site is analyzed by dissection and the amount of adhesion is scored using scoring systems published in the scientific literature for this type of injury.

Exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 60

### Tendon Surgical Adhesions Model to Assess Fibrosis Inhibiting Drug Combinations in Rabbits

This model is used to investigate whether adhesion of the tendons can be prevented by local slow release of drugs known to inhibit fibrosis. Polymeric formulations are loaded with the drug combinations and implanted around injured tendons in rabbits. In animals not treated with fibrosis-inhibiting drug combinations, adhesions develop within 3 weeks of flexor tendon injury if immobilization of the tendon is maintained during that period. An advantage of rabbits is that their tendon anatomy and cellular behaviour during tendon healing are similar to those in man except for the rate of healing that is much faster in rabbits.

Rabbits are anesthetized and the skin over the right hindlimb is shaved and prepared for sterile surgery. Sterile surgery is performed aided by an operating microscope. A longitudinal midline skin incision is made on the volvar aspect of the proximal phalange in digits 2 and 4. The synovial sheath of the tendons is carefully exposed and incised transversally to access the flexor digitorum profundus distal to the flexor digitorum superficialis bifurcation. Tendon injury is performed by gently lifting the flexor digitorum profundus with curved forceps and incising transversally through half of its substance. The formulation containing the drug combination is applied around the tendons in the sheath of one of the two digits randomly selected. The other digit is left untreated and is used as a control. The sheath is then repaired with 6-0 nylon suture. An immobilizing 6-0 nylon suture is inserted through the transverse metacarpal ligament into the tendon/sheath complex to immobilize the tendon and the sheath as a single unit to encourage adhesion formation. The wound is closed with 4-0 interrupted sutures. A bandage is applied around the hindpaw to further augment immobilization of the digits and ensure comfort and ambulation of the animals. The animals are recovered and returned to their cage.

Three weeks after surgery, the animals are anesthetized. After a skin incision, the tissue plane around the synovial sheath is dissected and the tendon-sheath complex harvested en block and transferred in 10% phosphate buffered formaldehyde for histopathology analysis. The animals are then euthanized. After paraffin embedding, serial 5-um thin cross-sections are cut every 2 mm through the sheath and tendon complex. Sections are stained with H&E and Movat's stains to evaluate adhesion growth. Each slide is digitized using a computer connected to a digital microscope camera (Nikon Micropublisher cooled camera). Morphometry analysis is then performed using image analysis software (ImagePro). Thickness and area of adhesion defined as the substance obliterating the synovial space are measured and compared between formulation-treated and control animals.

Exemplary drug combinations, or individual components thereof, that may be tested in this model include, but are not limited to: amoxapine and prednisolone, paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, terbinafine and manganese sulfate, or individual components of the above combinations.

### Example 61

### Drug-Loading a Screw-in Electrical Lead—Amoxapine and Prednisolone/Polymer Spraying—Dual Coating

A polyurethane solution (CHRONOFLEX AL 85 A) is prepared by dissolving 15 g of the polyurethane in 200 ml tetrahydrofuran (THF). 10 ml aliquots of this solution are placed in 20 ml glass scintillation vials. 1 mg, 5 mg, 10 mg, 20 mg, 50 mg, 75 mg, 100 mg, 200 mg, and 500 mg amoxapine are then added to each of the vials respectively. The solutions are tumbled for 3 hours at 20 rpm. The tip of an electrical pacing lead that comprises a screw in electrode tip (e.g., CAPSUREFIX NOVUS 5076) is screwed into the end of a silastic rod until the screw-in portion is completely incorporated into the silastic rod. The silastic rod it the attached to an overhead stirrer and the stir speed is set at 40 rpm. The 0.1 mg/ml amoxapine solution is placed in a 3 ml glass syringe that is then attached to an ultrasonic spray head (Sonus, Inc). The syringe is placed in a syringe pump. The solution is then sprayed onto the tip portion of the lead at a flow rate of 0.5 ml/min. Once the electrical lead tip is evenly coated with a amoxapine/polymer solution, the spraying is stopped and the coating is allowed to air dry for 1 hour. The spraying process is repeated using a series of prednisolone/polymers that are prepared in a similar manner as described for the amoxapine/polymer solutions. The electrode is unscrewed from the silastic rod. The electrode is further dried under vacuum for 24 hours. The process is repeated for all the prepared amoxapine solutions and prednisolone solutions using a fresh electrode each time. This order in which the drugs are applied to the device can be altered.

Exemplary drug combinations, or individual components thereof, that may be used to load the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 62

### Drug-Loading a Screw-in Electrical Lead—Drug Combination/Polymer Spraying with Top Coat

A polyurethane solution (CHRONOFLEX AL 85 A) is prepared by dissolving 15 g of the polyurethane in 200 ml tetrahydrofuran (THF). 10 ml aliquots of this solution are placed in 20 ml glass scintillation vials. One mg, 5 mg, 10 mg, 20 mg, 50 mg, 75 mg, 100 mg, 200 mg, and 500 mg each of amoxapine and prednisolone are then added to each of the vials respectively. The solutions are tumbled for 3 hours at 20 rpm. The tip of an electrical pacing lead that comprises a screw in electrode tip (e.g. CAPSUREFIX NOVUS 5076) is screwed into the end of a silastic rod until the screw-in portion is completely incorporated into the silastic rod. The silastic rod it the attached to an overhead stirrer and the stir speed is set at 40 rpm. The 0.1 mg/ml amoxapine/prednisolone solution is placed in a 3 ml glass syringe that is then attached to an ultrasonic spray head (Sonus, Inc). The syringe is placed in a syringe pump. The solution is then sprayed onto the tip portion of the lead at a flow rate of 0.5 ml/min. Once the electrical lead tip is evenly coated with drug/polymer solution, the spraying is stopped and the coating is allowed to air dry for 1 hour. Once the device is dried, the device is spray coated as described above using a 5% (w/v) solution of the polyurethane polymer in THF.

The electrode is unscrewed from the silastic rod. The electrode is further dried under vacuum for 24 hours. The process is repeated for all the prepared amoxapine/prednisolone solutions using a fresh electrode each time.

Exemplary drug combinations, or individual components thereof, that may be used to load the device include, but are not limited to: paroxetine and prednisolone, dipyridamole and prednisolone, dexamethasone and econazole, diflorasone and alprostadil, dipyridamole and amoxapine, dipyridamole and ibudilast, nortriptyline and loratadine (or desloratadine), albendazole and pentamidine, itraconazole and lovastatin, or individual components of the above combinations.

### Example 63

### Effects of the Combination of Methyl Prednisolone Acetate and Amoxapine in a Rat Carrageenan-Induced Paw Edema Model

A dose-range finding study was performed to determine the anti-inflammatory activity of various ratios of methyl prednisolone acetate and amoxapine in a rat carrageenan-induced paw edema model. The end points of assessment included inhibition of paw swelling at the time of maximum swelling (Tₘₐₓ=6 hours) and down regulation of the pro-inflammatory cytokine TNF-α in the paw tissue. The molar ratio of methyl prednisolone acetate to amoxapine ranged from 1:1 to 1:300, using total doses of methyl prednisolone acetate of 0.01, 0.03 or 0.1 mg/kg.

The test agent (a combination of methyl prednisolone acetate and amoxapine), vehicle control, or reference agents (methyl prednisolone acetate, amoxapine, or Depo-Medrol®) were administered in the left hind foot pad of rats. After 60 minutes, paw edema was induced by injection of 100 μl of 1% carrageenan in the same foot pad. The paw volume was measured with a water displacement plethysmometer immediately prior to test agent injection (T₋₁ₕ), at the time of carrageenan injection (T₀ₕ), and at T₆ₕ. Animals were euthanized by carbon dioxide inhalation. Paw tissue samples were collected and flash frozen in liquid nitrogen. Samples were assayed for TNF-α by enzyme-linked immunoassay (ELISA). The data are shown in Table 14.

TABLE 14
Results of Carrageenan-Induced Paw Edema Study

| | Edema^{b} ± SEM | | TNF-α^{d} ± SEM | |
|---|---|---|---|---|
| Groups^{a} | (%) | p-value^{c} | (pg/g) | p-value^{e} |
| Vehicle (diluent, negative control) | 49.6 ± 4.4 | — | 59.9 ± 13.1 | — |
| Depo-Medrol (positive control) 1 mg/kg | 15.3 ± 3.0 | <0.001 | 21.9 ± 6.3 | 0.01 |
| Amoxapine 2.26 mg/kg | 38.1 ± 3.3 | NS | 32.6 ± 10.1 | 0.05 |
| MePredAc 0.01 mg/kg | 32.6 ± 5.3 | 0.03 | 35.9 ± 11.3 | 0.001 |
| MePredAc 0.03 mg/kg | 26.2 ± 7.0 | 0.02 | 19.2 ± 3.1 | 0.01 |
| MePredAc 0.1 mg/kg | 12.2 ± 1.8 | <0.001 | 28.0 ± 6.0 | 0.06 |
| MePredAc 0.01 mg/kg + Amox 2.26 mg/kg | 48.4 ± 3.8 | NS | 47.5 ± 8.8 | NS |
| MePredAc 0.03 mg/kg + Amox 0.753 mg/kg | 24.3 ± 4.5 | 0.001 | 27.8 ± 3.7 | 0.04 |
| MePredAc 0.03 mg/kg + Amox 2.26 mg/kg | 13.6 ± 1.7 | <0.001 | 14.6 ± 4.1 | 0.01 |
| MePredAc 0.1 mg/kg + Amox 0.753 mg/kg | 22.2 ± 6.6 | 0.01 | 22.5 ± 5.7 | 0.01 |
| MePredAc 0.1 mg/kg + Amox 2.26 mg/kg | 12.5 ± 2.2 | <0.001 | 9.4 ± 2.6 | 0.01 |

^{a}All animals pre-treated with drugs at T-1 hr, at T0 hrs animals were injected with 1% Carrageenan (100 μl) by local injection into the paws. Vehicle Group n = 11 rats/group, other groups at n = 8 rats/group.
^{b}% Edema following carrageenan induction at Tmax = 6 hrs, SEM = standard error of the mean
^{c}p-value for edema vs. vehicle control, NS = not significant
^{d}TNF-α measured by ELISA in the paw tissues of carrageenan injected paws
^{e}p-value for TNF-α vs. vehicle control, NS = not significant

Carrageenan-injected paws treated with the vehicle (control) exhibited a ˜50% increase in paw volume. Administration of the clinical agent Depo-Medrol (1 mg/kg) significantly inhibited paw edema (p<0.001) reducing it to the background level of ˜15%. Treatment with amoxapine (Amox) alone at 2.26 mg/kg was not significantly different from the vehicle treatment. Groups treated with methyl prednisolone acetate (MePredAc) alone showed a dose-dependent reduction in paw edema following treatment.

Combinations containing 0.03 or 0.1 mg/kg MePredAc with higher amoxapine doses of 2.26 mg/kg significantly enhanced MePredAc effects, bringing down the edema levels to 13.6%±1.7 and 12.5%±2.2 respectively (p<0.001). This is equivalent to the effect observed using Depo-Medrol®, but with a much lower steroid dose. The levels of the pro-inflammatory cytokine TNF-α in the paw tissues were in good correlation with the reduction in paw edema.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing, it is appreciated that, although specific embodiments of the invention have been described herein for purposes of illustration, various modifications may be made without deviating from the spirit and scope of the invention. Accordingly, the invention is not limited except as by the appended claims.

What is claimed is:

1. A medical device comprising an electrical device and an anti-scarring drug combination;
wherein said electrical device is selected from the group consisting of: a neurostimulator, a sacral nerve stimulator, a gastric nerve stimulator, a cochlear implant, a bone growth stimulator, a cardiac pacemaker, an implantable cardioverter defibrillator system, a vagus nerve stimulator, an electrical lead, and a cardiac rhythm management device;
wherein said anti-scarring drug combination is selected from: amoxapine and prednisolone; paroxetine and prednisolone; dipyridamole and prednisolone; dexamethasone and econazole; diflorasone and alprostadil; dipyridamole and amoxapine; dipyridamole and ibudilast; nortriptyline and loratadine; nortriptyline and desloratadine; albendazole and pentamidine; itraconazole and lovastatin; terbinafine and manganese sulfate; a triazole and an aminopyridine, an antiprotozoal and a diaminopyridine, an antiprotozoal and a quaternary ammonium compound; an aromatic diamidine and a compound selected from the group consisting of: an antiestrogen, an anti-fungal imidazole, disulfuram, and ribavirin; an aminopyridine and a compound selected from the group consisting of: phenothiazine, dacarbazine, or phenelzine; a quaternary ammonium compound and a compound selected from the group consisting of: an anti-fungal imidazole, halopnogin, MnSO₄, and ZnCl₂; an antiestrogen and at least one compound selected from the group consisting of: phenothiazine, cupric chloride, dacarbazine, methoxsalen, and phenelzine; an antifungal imidazone and at least one compound selected from a group consisting of: disulfuram and ribavirin; an estrogenic compound and dacarbazine; amphotericin B and dithiocarbamoyl disulfide; terbinafine and a manganese compound; a tricyclic antidepressant and a corticosteroid; a tetra-substituted pyrimidopyrimidine and a corticosteroid; a prostaglandin and a retinoid; an azole and a steroid; a steroid and a compound selected from the group consisting of: a prostaglandin, a beta-adrenergic receptor ligand, an anti-mitotic agent, and a microtubule inhibitor; a corticosteroid and either a serotonin norepinephrine reuptake inhibitor or a naradrenaline reuptake inhibitor; a non-steroidal immunophilin-dependent immunosuppressant and a non-steroidal immunophilin-dependent immunosuppressant enhancer; an antihistamine and a compound selected from the group consisting of a corticosteroid, a tricyclic antidepressant, a tetracyclic antidepressant, a selective serotonin reuptake inhibitor, and a steroid receptor modulator; a tricyclic compound and a corticosteroid; an antipsychotic drug and an antiprotozoal drug; an antihelmintic drug and an antiprotozoal drug; ciclopirox and an antiproliferative agent; a salicylanilide and an antiproliferative agent; pentamidine and chlorpromazine; an antihelmintic drug and an antiprotozoal drug; dibucaine and a vinca alkaloid; an amide local anaesthetic related to bupivacaine and a vinca alkaloid; pentamidine and an antiproliferative agent; a triazole and an antiarrhythmic agent; an azole and an HMG-CoA reductase inhibitor; a phenothiazine conjugate; phenothiazine and an antiproliferative agent; a kinesin inhibitor and an antiproliferative agent; an agent that reduces the biological activity of a mitotic kinesin and an agent that reduces the biological activity of protein tyrosine phosphatase; an anti-inflammatory agent and an agent selected from group consisting of an anti-depressant, an SSRI, a cardiovascular agent, an anti-fungal agent, and prostaglandin; a cardiovascular drug and an antidepressant; a cardiovascular drug and a phosphodiesterase IV inhibitor; an antidepressant and an antihistamine; an anti-fungal agent and an HMG-CoA reductase inhibitor; and an antifungal agent and a metal ion; and
wherein said anti-scarring drug combination inhibits scarring between said electrical device and a host into which said electrical device is implanted.

2. A method for inhibiting scarring comprising placing an electrical device and an anti-scarring drug combination into an animal host,
wherein said electrical device is selected from the group consisting of: a neurostimulator, a sacral nerve stimulator, a gastric nerve stimulator, a cochlear implant, a bone growth stimulator, a cardiac pacemaker, an implantable cardioverter defibrillator system, a vagus nerve stimulator, an electrical lead, and a cardiac rhythm management device;
wherein said anti-scarring drug combination is selected from: amoxapine and prednisolone; paroxetine and prednisolone; dipyridamole and prednisolone; dexamethasone and econazole; diflorasone and alprostadil; dipyridamole and amoxapine; dipyridamole and ibudilast; nortriptyline and loratadine; nortriptyline and desloratadine; albendazole and pentamidine; itraconazole and lovastatin; terbinafine and manganese sulfate; a triazole and an aminopyridine, an antiprotozoal and a diaminopyridine, an antiprotozoal and a quaternary ammonium compound; an aromatic diamidine and a compound selected from the group consisting of: an antiestrogen, an anti-fungal imidazole, disulfuram, and ribavirin; an aminopyridine and a compound selected from the group consisting of: phenothiazine, dacarbazine, or phenelzine; a quaternary ammonium compound and a compound selected from the group consisting of: an anti-fungal imidazole, halopnogin, MnSO₄, and ZnCl₂; an antiestrogen and at least one compound selected from the group consisting of: phenothiazine, cupric chloride, dacarbazine, methoxsalen, and phenelzine; an antifungal imidazone and at least one compound selected from a group consisting of: disulfuram and ribavirin; an estrogenic compound and dacarbazine; amphotericin B and dithiocarbamoyl disulfide; terbinafine and a manganese compound; a tricyclic antidepressant and a corticosteroid; a tetra-substituted pyrimidopyrimidine and a corticosteroid; a prostaglandin and a retinoid; an azole and a steroid; a steroid and a compound selected from the group consisting of: a prostaglandin, a beta-adrenergic receptor ligand, an anti-mitotic agent, and a microtubule inhibitor; a corticosteroid and either a serotonin norepinephrine reuptake inhibitor or a naradrenaline reuptake inhibitor; a non-steroidal immunophilin-dependent immunosuppressant and a non-steroidal immunophilin-dependent immunosuppressant enhancer; an antihistamine and a compound selected from the group consisting of a corticosteroid, a tricyclic antidepressant, a tetracyclic antidepressant, a selective serotonin reuptake inhibitor, and a steroid receptor modulator; a tricyclic compound and a corticosteroid; an antipsychotic drug and an antiprotozoal drug; an antihelmintic drug and an antiprotozoal drug; ciclopirox and an antiproliferative agent; a salicylanilide and an antiproliferative agent; pentamidine and chlorpromazine; an antihelmintic drug and an antiprotozoal drug; dibucaine and a vinca alkaloid; an amide local anaesthetic related to bupivacaine and a vinca alkaloid; pentamidine and an antiproliferative agent; a triazole and an antiarrhythmic agent; an azole and an HMG-CoA reductase inhibitor; a phenothiazine conjugate; phenothiazine and an antiproliferative agent; a kinesin inhibitor and an antiproliferative agent; an agent that reduces the biological activity of a mitotic kinesin and an agent that reduces the biological activity of protein tyrosine phosphatase; an anti-inflammatory agent and an agent selected from group consisting of an anti-depressant, an SSRI, a cardiovascular agent, an anti-fungal agent, and prostaglandin; a cardiovascular drug and an antidepressant; a cardiovascular drug and a phosphodiesterase IV inhibitor; an antidepressant and an antihistamine; an anti-fungal agent and an HMG-CoA reductase inhibitor; and an antifungal agent and a metal ion; and
wherein said anti-scarring drug combination inhibits scarring.

3. A method for making a medical device comprising combining an electrical device and an anti-scarring drug combination,
wherein said electrical device is selected from the group consisting of: a neurostimulator, a sacral nerve stimulator, a gastric nerve stimulator, a cochlear implant, a bone growth stimulator, a cardiac pacemaker, an implantable cardioverter defibrillator system, a vagus nerve stimulator, an electrical lead, and a cardiac rhythm management device;
wherein said anti-scarring drug combination is selected from: amoxapine and prednisolone; paroxetine and prednisolone; dipyridamole and prednisolone; dexamethasone and econazole; diflorasone and alprostadil; dipyridamole and amoxapine; dipyridamole and ibudilast; nortriptyline and loratadine; nortriptyline and desloratadine; albendazole and pentamidine; itraconazole and lovastatin; terbinafine and manganese sulfate; a triazole and an aminopyridine, an antiprotozoal and a diaminopyridine, an antiprotozoal and a quaternary ammonium compound; an aromatic diamidine and a compound selected from the group consisting of: an antiestrogen, an anti-fungal imidazole, disulfuram, and ribavirin; an aminopyridine and a compound selected from the group consisting of: phenothiazine, dacarbazine, or phenelzine; a quaternary ammonium compound and a compound selected from the group consisting of: an anti-fungal imidazole, halopnogin, MnSO₄, and ZnCl₂; an antiestrogen and at least one compound selected from the group consisting of: phenothiazine, cupric chloride, dacarbazine, methoxsalen, and phenelzine; an antifungal imidazone and at least one compound selected from a group consisting of: disulfuram and ribavirin; an estrogenic compound and dacarbazine; amphotericin B and dithiocarbamoyl disulfide; terbinafine and a manganese compound; a tricyclic antidepressant and a corticosteroid; a tetra-substituted pyrimidopyrimidine and a corticosteroid; a prostaglandin and a retinoid; an azole and a steroid; a steroid and a compound selected from the group consisting of: a prostaglandin, a beta-adrenergic receptor ligand, an anti-mitotic agent, and a microtubule inhibitor; a corticosteroid and either a serotonin norepinephrine reuptake inhibitor or a naradrenaline reuptake inhibitor; a non-steroidal immunophilin-dependent immunosuppressant and a non-steroidal immunophilin-dependent immunosuppressant enhancer; an antihistamine and a compound selected from the group consisting of a corticosteroid, a tricyclic antidepressant, a tetracyclic antidepressant, a selective serotonin reuptake inhibitor, and a steroid receptor modulator; a tricyclic compound and a corticosteroid; an antipsychotic drug and an antiprotozoal drug; an antihelmintic drug and an antiprotozoal drug; ciclopirox and an antiproliferative agent; a salicylanilide and an antiproliferative agent; pentamidine and chlorpromazine; an antihelmintic drug and an antiprotozoal drug; dibucaine and a vinca alkaloid; an amide local anaesthetic related to bupivacaine and a vinca alkaloid; pentamidine and an antiproliferative agent; a triazole and an antiarrhythmic agent; an azole and an HMG-CoA reductase inhibitor; a phenothiazine conjugate; phenothiazine and an antiproliferative agent; a kinesin inhibitor and an antiproliferative agent; an agent that reduces the biological activity of a mitotic kinesin and an agent that reduces the biological activity of protein tyrosine phosphatase; an anti-inflammatory agent and an agent selected from group consisting of an anti-depressant, an SSRI, a cardiovascular agent, an anti-fungal agent, and prostaglandin; a cardiovascular drug and an antidepressant; a cardiovascular drug and a phosphodiesterase IV inhibitor; an antidepressant and an antihistamine; an anti-fungal agent and an HMG-CoA reductase inhibitor; and an antifungal agent and a metal ion; and
wherein said anti-scarring drug combination inhibits scarring between said electrical device and a host into which said electrical device is implanted.

4. A method for implanting an electrical device comprising:
(a) infiltrating a tissue of a host where said electrical device is to be implanted with an anti-scarring drug combination; and
(b) implanting said electrical device into said host;
wherein said electrical device is selected from the group consisting of: a neurostimulator, a sacral nerve stimulator, a gastric nerve stimulator, a cochlear implant, a bone growth stimulator, a cardiac pacemaker, an implantable cardioverter defibrillator system, a vagus nerve stimulator, an electrical lead, and a cardiac rhythm management device; and
wherein said anti-scarring drug combination is selected from: amoxapine and prednisolone; paroxetine and prednisolone; dipyridamole and prednisolone; dexamethasone and econazole; diflorasone and alprostadil; dipyridamole and amoxapine; dipyridamole and ibudilast; nortriptyline and loratadine; nortriptyline and desloratadine; albendazole and pentamidine; itraconazole and lovastatin; terbinafine and manganese sulfate; a triazole and an aminopyridine, an antiprotozoal and a diaminopyridine, an antiprotozoal and a quaternary ammonium compound; an aromatic diamidine and a compound selected from the group consisting of: an antiestrogen, an anti-fungal imidazole, disulfuram, and ribavirin; an aminopyridine and a compound selected from the group consisting of: phenothiazine, dacarbazine, or phenelzine; a quaternary ammonium compound and a compound selected from the group consisting of: an anti-fungal imidazole, halopnogin, MnSO₄, and ZnCl₂; an antiestrogen and at least one compound selected from the group consisting of: phenothiazine, cupric chloride, dacarbazine, methoxsalen, and phenelzine; an antifungal imidazone and at least one compound selected from a group consisting of: disulfuram and ribavirin; an estrogenic compound and dacarbazine; amphotericin B and dithiocarbamoyl disulfide; terbinafine and a manganese compound; a tricyclic antidepressant and a corticosteroid; a tetra-substituted pyrimidopyrimidine and a corticosteroid; a prostaglandin and a retinoid; an azole and a steroid; a steroid and a compound selected from the group consisting of: a prostaglandin, a beta-adrenergic receptor ligand, an anti-mitotic agent, and a microtubule inhibitor; a corticosteroid and either a serotonin norepinephrine reuptake inhibitor or a naradrenaline reuptake inhibitor; a non-steroidal immunophilin-dependent immunosuppressant and a non-steroidal immunophilin-dependent immunosuppressant enhancer; an antihistamine and a compound selected from the group consisting of a corticosteroid, a tricyclic antidepressant, a tetracyclic antidepressant, a selective serotonin reuptake inhibitor, and a steroid receptor modulator; a tricyclic compound and a corticosteroid; an antipsychotic drug and an antiprotozoal drug; an antihelmintic drug and an antiprotozoal drug; ciclopirox and an antiproliferative agent; a salicylanilide and an antiproliferative agent; pentamidine and chlorpromazine; an antihelmintic drug and an antiprotozoal drug; dibucaine and a vinca alkaloid; an amide local anaesthetic related to bupivacaine and a vinca alkaloid; pentamidine and an antiproliferative agent; a triazole and an antiarrhythmic agent; an azole and an HMG-CoA reductase inhibitor; a phenothiazine conjugate; phenothiazine and an antiproliferative agent; a kinesin inhibitor and an antiproliferative agent; an agent that reduces the biological activity of a mitotic kinesin and an agent that reduces the biological activity of protein tyrosine phosphatase; an anti-inflammatory agent and an agent selected from group consisting of an anti-depressant, an SSRI, a cardiovascular agent, an anti-fungal agent, and prostaglandin; a cardiovascular drug and an antidepressant; a cardiovascular drug and a phosphodiesterase IV inhibitor; an antidepressant and an antihistamine; an anti-fungal agent and an HMG-CoA reductase inhibitor; and an antifungal agent and a metal ion.
